(19) United States
(12) Patent Application Publication
(10) Pub. No.: US 2026/0216175 A1
(43) Pub. Date: Jul. 30, 2026

(54) COMPLEMENT INHIBITION

(71) Applicant: Apellis Pharmaceuticals, Inc., Waltham, MA (US)

(72) Inventors: Andrew Simon Cook, Stow, MA (US); Andrew Edward Carvalho, Lowell, MA (US); Michael Jon Reid, Freeland, WA (US); Johannes Wilhelm Georg Meissner, Groningen (NL); Edwin Kroon, Zuidhorn (NL); Paul Ruben Werkhoven, Groningen (NL); Eduardo Moreno Saavedra, Groningen (NL); Vincent Lars de Boer, Aduard (NL); Guangyun Yu, Groningen (NL)

(21) Appl. No.: 19/145,748

(22) PCT Filed: Jan. 5, 2024
(86) PCT No.: PCT/US2024/010501

§ 371 Date: Jul. 3, 2025

Related U.S. Application Data
(60) Provisional application No. 63/437,359, filed on Jan. 5, 2023.

(51) Int. Cl. A61K 31/496; A61K 31/4985; A61K 31/538; A61K 31/551; C07D 403/06; C07D 403/14; C07D 405/14; C07D 413/14; C07D 471/04; C07D 487/04

(52) U.S. Cl. CPC A61K 31/496; A61K 31/4985; A61K 31/538; A61K 31/551; C07D 403/06; C07D 403/14; C07D 405/14; C07D 413/14; C07D 471/04; C07D 487/04

(57) ABSTRACT

Among other things, the present disclosure provides technologies for modulating complement activation. In some embodiments, the present disclosure provides compounds that inhibit C3 convertase activity. In some embodiments, the present disclosure provides methods for treating complement-mediated conditions, disorders or diseases. In some embodiments, the present disclosure provides methods for treating conditions, disorders or diseases mediated by C3 convertase.

### CROSS-REFERENCE TO RELATED APPLICATIONS

This application claims priority to U.S. Provisional Application No. 63/437,356, filed Jan. 5, 2023, the entirety of which is incorporated herein by reference.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

Among other things, the present disclosure provides technologies (e.g., compounds, compositions, methods, etc.) useful for treating various conditions, disorders or diseases. In some embodiments, the present disclosure provides technologies for preventing or treating conditions, disorders or diseases associated with complement activation. In some embodiments, a condition, disorder or disease is associated with alternative complement activation. In some embodiments, a condition, disorder or disease is associated with C3 convertase. In some embodiments, a condition, disorder or disease is associated with factor B.

In some embodiments, the present disclosure provides a compound having the structure of formula I:

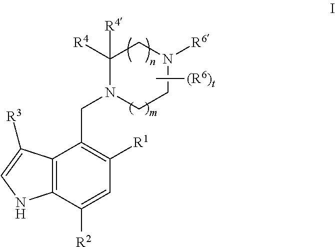

or a pharmaceutically acceptable salt thereof, wherein:
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R⁵ and further optionally substituted with one or two R⁵′;
each R⁵′ is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R⁵′ are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{4′} is hydrogen, C₁-C₄ alkyl, or hydroxy C₁-C₄ alkyl;
R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

Among other things, provided compounds, e.g., those of formula I or a pharmaceutically acceptable salt thereof, are particularly useful for modulating C3 convertase activity. In some embodiments, provided compounds are useful for modulating complement activation. In some embodiments, the present disclosure provides a method for modulating a C3 convertase activity, comprising contacting a C3 convertase with a provided compound. In some embodiments, the present disclosure provides a method for modulating a C3 convertase activity, comprising administering to a system comprising a C3 convertase a provided compound. In some embodiments, the present disclosure provides a method for modulating a C3 convertase activity, comprising administering to a subject expressing or comprising a C3 convertase a provided compound. In some embodiments, an activity of a C₃ convertase is inhibited. In some embodiments, a provided compound is useful as a C3 convertase inhibitor. In some embodiments, a C3 convertase is or comprises factor B. In some embodiments, a C3 convertase is factor B. In some embodiments, a C3 convertase forms a complex with one or more polypeptides.

In some embodiments, the present disclosure provides methods for preventing a condition, disorder or disease, comprising administering to a subject susceptible thereto an effective amount of a provided compound (e.g., a compound of formula I or a pharmaceutically acceptable salt thereof).

In some embodiments, the present disclosure provides methods for treating a condition, disorder or disease, comprising administering to a subject suffering therefrom an effective amount of a provided compound (e.g., a compound of formula I or a pharmaceutically acceptable salt thereof).

Various conditions, disorders or diseases associated with complement activation can prevented or treated utilizing provided technologies in accordance with the present disclosure. In some embodiments, a condition, disorder or disease is associated with complement activation. In some embodiments, a condition, disorder or disease is associated with alternative complement activation. In some embodiments, a condition, disorder or disease is associated with C3 convertase. In some embodiments, a subject who is suffering from a condition, disorder or disease can benefit from inhibition of a C₃ convertase.

In some embodiments, a condition, disorder or disease is selected from age-related macular degeneration (e.g., intermediate age-related macular degeneration), geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.

In some embodiments a subject with hemolytic anemia suffers from paroxysmal nocturnal hemoglobinuria. In some embodiments a subject with hemolytic anemia suffers from autoimmune hemolytic anemia (e.g., cold agglutinin disease or warm autoimmune hemolytic anemia). In some embodiments a subject suffers from TMA secondary to atypical hemolytic uremic syndrome.

In some embodiments, a condition, disorder or disease, e.g., a complement-mediated disorder, is complement activation secondary to administration of another agent, e.g., a therapeutic or diagnostic agent. For example, in some embodiments, a complement-mediated disorder is complement activation secondary to gene therapy (e.g., gene therapy with a viral vector such as an adeno-associated virus (AAV), adenovirus, or lentivirus vector) or complement activation secondary to cell therapy. In some embodiments, a subject suffers from TMA secondary to hematopoietic stem cell transplant (HSCT-TMA). In some embodiments, a subject suffers from drug-induced TMA. In some embodiments, administration of a compound described herein prior to and/or following administration of another therapeutic agent may increase the efficacy and/or safety of said therapeutic agent. In some embodiments, the present disclosure provides methods for improving efficacy and/or safety of a therapeutic agent, comprising administering to a subject an effective amount of a provided compound (e.g., a compound of formula I or a pharmaceutically acceptable salt thereof) prior to, concurrently with (either in the same or different composition) or subsequently to administration of the therapeutic agent to the subject. In some embodiments, the present disclosure provides improved administration (e.g., dosage regimen, unit doses, total doses, improved intervals, durations of treatment, etc.) of a therapeutic agent, comprising administering to a subject a provided compound (e.g., a compound of formula I or a pharmaceutically acceptable salt thereof) prior to, concurrently with (either in the same or different composition) or subsequently to administration of the therapeutic agent to the subject, wherein the administration of the therapeutic agent provides improved efficacy and/or safety compared to a reference administration (e.g., administration of the therapeutic agent without administration of a provided compound (e.g., a compound of formula I or a pharmaceutically acceptable salt thereof)).

In some embodiments, a subject has a defect in complement regulation, optionally wherein the defect comprises abnormally low expression of one or more complement regulatory proteins by at least some of the subject's cells.

In some embodiments, a complement-mediated disorder is a chronic disorder. In some embodiments, a complement-mediated disorder involves complement-mediated damage to red blood cells, optionally wherein the disorder is paroxysmal nocturnal hemoglobinuria or atypical hemolytic uremic syndrome. In some embodiments, a complement-mediated disorder is an autoimmune disease, optionally wherein the disorder is multiple sclerosis. In some embodiments, a complement-mediated disorder involves kidney, optionally wherein the disorder is membranoproliferative glomerulonephritis, lupus nephritis, IgA nephropathy (IgAN), primary membranous nephropathy (primary MN), C3 glomerulopathy (C3G), or acute kidney injury. In some embodiments, a complement-mediated disorder involves the central or peripheral nervous system or neuromuscular junction, optionally wherein a disorder is neuromyelitis optica, Guillain-Barré syndrome, multifocal motor neuropathy, or myasthenia gravis. In some embodiments, a complement-mediated disorder involves the respiratory system, optionally wherein the disorder is characterized by pulmonary fibrosis. In some embodiments, a complement-mediated disorder involves the vascular system, optionally wherein the disorder is characterized by vasculitis.

Certain conditions, disorders or diseases that can be prevented or treated by provided technologies, e.g., conditions, disorders or diseases mediated by complement activation (in some embodiments, alternative complement activation), conditions, disorders or diseases mediated by C3 convertase, conditions, disorders or diseases mediated by factor B, etc. are reported in, e.g., WO 2015/009616, WO 2019/043609, WO 2022/143845, WO 2022/028527, WO 2022/028507, etc. In some embodiments, a condition, disorder or disease is a renal condition, disorder or disease. In some embodiments, a condition, disorder or disease is complement-driven renal disease C3G (C3 glomerulopathy). In some embodiments, a condition, disorder or disease is c IgAN (immunoglobuline A nephropathy). In some embodiments, a condition, disorder or disease is a nephropathy. In some embodiments, a condition, disorder or disease is a nephropathy with evidence of glomerular C3 deposition. In some embodiments, a condition, disorder or disease is membranous nephropathy. In some embodiments, a condition, disorder or disease is HUS (E. coli induced hemolytic uremic syndrome).

In some embodiments, a compound (e.g., a compound of formula I or a pharmaceutically acceptable salt thereof) is provided in a pharmaceutical composition. In some embodiments, the present disclosure provides a pharmaceutical composition comprising a provided compound and a pharmaceutically acceptable carrier. In some embodiments, the present disclosure provides a pharmaceutical composition comprising a compound of formula I or a pharmaceutically acceptable salt thereof and a pharmaceutically acceptable carrier. In some embodiments, a composition may comprise two or more pharmaceutically acceptable salt forms of a compound. In some embodiments, a composition comprises two or more pharmaceutically acceptable salts of a compound of formula I. In some embodiments, the present disclosure provides a pharmaceutical composition which delivers a provided compound (e.g., a compound of formula I or a pharmaceutically acceptable salt thereof) and comprises a pharmaceutically acceptable carrier. In some embodiments, the present disclosure provides a pharmaceutical composition comprises a compound of formula I or a pharmaceutically acceptable salt dissolved in a pharmaceutically acceptable liquid, e.g., water or a pharmaceutically acceptable buffer.

Various technologies for administering compounds or compositions can be utilized in accordance with the present disclosure. For example, in some embodiments, provided compounds or compositions are administered orally. In some embodiments, provided compounds or compositions are administered intravenously.

In some embodiments, the composition is administered intravenously to the subject. In some embodiments, the composition is administered to a hepatocyte of the subject. In some embodiments, the composition is administered to the hepatocyte ex vivo. In some embodiments, the composition is administered to the hepatocyte in vivo.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in, e.g., “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Concurrent administration: As used herein, the term “concurrent administration” with respect to two or more agents, e.g., therapeutic agents, is administration performed using doses and time intervals such that the administered agents are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc.). Accordingly, the agents may, but need not be, administered together as part of a single composition. In addition, the agents may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). According to various embodiments of the disclosure, agents administered within such time intervals may be considered to be administered at substantially the same time. In certain embodiments of the disclosure, concurrently administered agents are present at effective concentrations within the body (e.g., in the blood and/or at a site of local complement activation) over the time interval. When administered concurrently, the effective concentration of each of the agents needed to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. The non-negligible concentration of an agent may be, for example, less than approximately 5% of the concentration that would be required to elicit a particular biological response, e.g., a desired biological response.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4 dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothicnyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Local administration: As used herein, the term “local administration” or “local delivery”, in reference to delivery of a composition or agent, refers to delivery that does not rely upon transport of the composition or agent to its intended target tissue or site via the vascular system. The composition or agent may be delivered directly to its intended target tissue or site, or in the vicinity thereof, e.g., in close proximity to the intended target tissue or site. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site. It will be understood that once having been locally delivered a fraction of a therapeutic agent (typically only a minor fraction of the administered dose) may enter the vascular system and be transported to another location, including back to its intended target tissue or site.

Local complement activation: As used herein, the term “local complement activation” refers to complement activation that occurs outside the vascular system.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is optionally substituted C₁-C₆ alkyl) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4 nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmcthyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4 dimethoxy-6-nitrobenzyl carbamate, phenyl (o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3 (o-nitrophenyl)propanamide, 2-methyl-2 (o nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmalcimidc, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4 (4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4 methoxypiperidin-4-yl (CTMP), 1,4 dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4 dinitrophenyl, benzyl, p-methoxybenzyl, 3,4 dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4 (4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4 (ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4 dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4 methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4 dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidenc derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl) xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Sequential administration: As used herein, the term “Sequential administration” of two or more agents refers to administration of two or more agents to a subject such that the agents are not present together in the subject's body, or at a relevant site of activity in the body, at greater than non-negligible concentrations. Administration of the agents may, but need not, alternate. Each agent may be administered multiple times.

Subject: As used herein, the term “subject” or “test subject” refers to any organism to which a provided compound or composition is administered in accordance with the present disclosure, e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In many instances, provided compounds or compositions are administered or delivered to human subjects.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: As used herein, the term “systemic” in reference to complement components, refers to complement proteins that are synthesized by liver hepatocytes and enter the bloodstream, or are synthesized by circulating macrophages or monocytes and secreted into the bloodstream.

Systemic complement activation: As used herein, the term “systemic complement activation” is complement activation that occurs in the blood, plasma, or serum and/or involves activation of systemic complement proteins at many locations throughout the body, affecting many body tissues, systems, or organs.

Systemic administration: As used herein, the term “systemic administration” and like terms are used herein consistently with their usage in the art to refer to administration of an agent such that the agent becomes widely distributed in the body in significant amounts and has a biological effect, e.g., its desired effect, in the blood and/or reaches its desired site of action via the vascular system. Typical systemic routes of administration include administration by (i) introducing the agent directly into the vascular system or (ii) subcutaneous, oral, pulmonary, or intramuscular administration wherein the agent is absorbed, enters the vascular system, and is carried to one or more desired site(s) of action via the blood.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e, providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

As those skilled in the art will appreciate, methods and compositions described herein relating to provided compounds generally also apply to pharmaceutically acceptable salts of such compounds. Unless specified otherwise, solvates, stereoisomers, tautomers, salts of provided compounds are included.

### DETAILED DESCRIPTION OF CERTAIN EMBODIMENTS

Technologies of the present disclosure may be understood more readily by reference to the following detailed description of certain embodiments.

### I. Complement System

To facilitate understanding of the disclosure, and without intending to limit the provided technologies in any way, this section provides an overview of complement and its pathways of activation. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344(14): 1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3 (H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b: C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) arc involved in the pathogenesis of a variety of complement-mediated disorders, e.g., as discussed herein.

### II. Certain Provided Compounds and Composition

Among other things, the present disclosure provides compounds, e.g., those of formula I or pharmaceutically acceptable salts thereof, and compositions thereof:

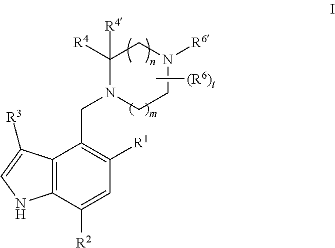

wherein each variable is independently as described herein.

In some embodiments, R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is hydrogen, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is hydrogen, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷.

In some embodiments, R¹ is hydrogen. In some embodiments, R¹ is halogen. In some embodiments, R¹ is hydroxy. In some embodiments, R¹ is C₁-C₆ alkyl. In some embodiments, R¹ is C₂-C₆ alkenyl. In some embodiments, R¹ is C₃-C₆ cycloalkyl. In some embodiments, R¹ is C₃-C₅ cycloalkyl. In some embodiments, R¹ is cyclopropyl.

In some embodiments, R¹ is halo C₁-C₆ alkyl.

In some embodiments, R¹ is hydroxy C₁-C₆ alkyl. In some embodiments, R¹ is amino C₁-C₆ alkyl. In some embodiments, R¹ is C₁-C₆ alkoxy C₁-C₆ alkyl. In some embodiments, R¹ is C₁-C₆ alkoxy C₁-C₆ alkoxy. In some embodiments, R¹ is C₃-C₆ cycloalkyl C₁-C₆ alkoxy.

In some embodiments, R¹ is halo C₁-C₆ alkoxy. In some embodiments, R¹ is halo C₁-C₄ alkoxy. In some embodiments, R¹ is halo C₁-C₂ alkoxy. In some embodiments, R¹ is halo C₁-C₂ alkoxy. In some embodiments, R¹ is —OCHF₂.

In some embodiments, R¹ is —S(O)ₚC₁-C₆ alkyl. In some embodiments, R¹ is —SC₁-C₆ alkyl. In some embodiments, R¹ is —S(O)C₁-C₆ alkyl. In some embodiments, R¹ is —CH₂NHC(O)C₁-C₄ alkyl. In some embodiments, R¹ is —OCH₂C(O)R⁷.

In some embodiments, R¹ is C₁-C₆ alkoxy.

As those skilled in the art appreciate, a C₁ to C₆ (e.g., C₁-C₂, C₁-C₃, C₁-C₄, C₁-C₅, C₁-C₆, etc.) alkyl group, either independently or as part of another group (e.g., as in C₁-C₆ alkoxy, C₁-C₆ alkoxy C₁-C₆ alkyl, etc.), can independently be a C₁, C₂, C₃, C₄, C₅, or C₆ alkyl group. For example, in some embodiments, C₁-C₆ alkyl is methyl. In some embodiments, it is ethyl. In some embodiments, it is propyl. In some embodiments, it is n-propyl. In some embodiments, it is isopropyl. In some embodiments, it is butyl. In some embodiments, it is pentyl. In some embodiments, it is hexyl. In some embodiments, R¹ is C₁-C₆ alkoxy. In some embodiments, R¹ is —O—CH₃.

In some embodiments, p is 0. In some embodiments, p is 1. In some embodiments, p is 2.

In some embodiments, R¹ is —SC₁-C₆ alkyl. In some embodiments, R¹ is —S(O)C₁-C₆ alkyl. In some embodiments, R¹ is —S(O)₂C₁-C₆ alkyl.

In some embodiments, R² is hydrogen, C₁-C₆ alkyl, C₁-C₆ alkoxy, hydroxy C₁-C₆ alkyl, or halogen.

In some embodiments, R² is hydrogen.

In some embodiments, R² is C₁-C₆ alkyl. In some embodiments, R² is methyl. In some embodiments, R² is ethyl.

In some embodiments, R² is C₁-C₆ alkoxy. In some embodiments, R² is hydroxy C₁-C₆ alkyl. In some embodiments, R² is halogen. In some embodiments, R² is —F. In some embodiments, R² is —Cl. In some embodiments, R² is —Br. In some embodiments, R² is —I.

In some embodiments, R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 1, or 2 C₁-C₄ alkyl groups, and wherein alkyl and haloalkyl are optionally substituted with 1 hydroxy group.

In some embodiments, R³ is hydrogen.

In some embodiments, R³ is halogen. In some embodiments, R³ is —F. In some embodiments, R³ is —Cl. In some embodiments, R³ is —Br. In some embodiments, R³ is —I. In some embodiments, R³ is cyano.

In some embodiments, R³ is C₁-C₄ alkyl. In some embodiments, R³ is C₁-C₄ alkyl optionally substituted with one hydroxy group. In some embodiments, R³ is C₁-C₄ alkyl substituted with one hydroxy group.

In some embodiments, R³ is halo C₁-C₄ alkyl. In some embodiments, R³ is halo C₁-C₄ alkyl optionally substituted with one hydroxy group. In some embodiments, R³ is halo C₁-C₄ alkyl substituted with one hydroxy group. In some embodiments, R³ is —CH₂C(O)R⁷.

In some embodiments, R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R⁵ and further optionally substituted with one or two R⁵′. In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxyl, and cyanomethyl.

In some embodiments, R⁴ is optionally substituted phenyl. In some embodiments, R⁴ is optionally substituted naphthyl. In some embodiments, R⁴ is optionally substituted 1,2,3,4-tetrahydronaphthalenyl. In some embodiments, R⁴ is optionally substituted 5-10 membered heteroaryl. In some embodiments, R⁴ is optionally substituted 5-6 membered heteroaryl. In some embodiments, R⁴ is optionally substituted 9-membered bicyclic heteroaryl. In some embodiments, R⁴ is optionally substituted 10-membered bicyclic heteroaryl. In some embodiments, R⁴ is not substituted with R⁵. In some embodiments, R⁴ is not substituted with R^{5′}. In some embodiments, R⁴ is not substituted with R⁵ or R⁵′. In some embodiments, R⁴ is substituted with R⁵. In some embodiments, R⁴ is substituted with R⁵ but no R⁵′. In some embodiments, R⁴ is substituted with R^{5′}. In some embodiments, R⁴ is substituted with one R^{5′}. In some embodiments, R⁴ is substituted with two R^{5′}. In some embodiments, R⁴ is substituted with R⁵ and R⁵′. In some embodiments, R⁴ is substituted with one R⁵ and one R⁵′. In some embodiments, R⁴ is substituted with one R⁵ and two R⁵′.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R⁵ and further substituted with 0 or 1 substituent selected from halogen, C₁-C₄ alkyl, C₁-C₄ alkoxy, hydroxy C₁-C₄ alkyl, hydroxyl, and cyanomethyl.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with —C(O)R⁸ and further substituted with 0 or 1 substituent selected from halogen, C₁-C₄ alkyl, C₁-C₄ alkoxy, hydroxy C₁-C₄ alkyl, hydroxyl, and cyanomethyl.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with —C(O)OH and further substituted with 0 or 1 substituent selected from halogen, C₁-C₄ alkyl, C₁-C₄ alkoxy, hydroxy C₁-C₄ alkyl, hydroxyl, and cyanomethyl.

In some embodiments, R⁴ is phenyl. In some embodiments, R⁴ is phenyl substituted with R⁵ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is phenyl substituted with R⁵ only. In some embodiments, R⁴ is phenyl substituted with R⁵ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is phenyl substituted with —C(O)R⁸ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is phenyl substituted with —C(O)R⁸ only. In some embodiments, R⁴ is phenyl substituted with —C(O)R⁸ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is phenyl substituted with —COOH and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is phenyl substituted with —COOH only. In some embodiments, R⁴ is phenyl substituted with —COOH and further substituted with one substituent selected from halogen, C₁-C₄ alkyl, C₁-C₄ alkoxy, hydroxy C₁-C₄ alkyl, hydroxyl, and cyanomethyl.

In some embodiments, R⁴ is naphthyl. In some embodiments, R⁴ is naphthyl substituted with R⁵ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is naphthyl substituted with R⁵ only. In some embodiments, R⁴ is naphthyl substituted with R⁵ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is naphthyl substituted with —C(O)R⁸ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is naphthyl substituted with —C(O)R⁸ only. In some embodiments, R⁴ is naphthyl substituted with —C(O)R⁸ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is naphthyl substituted with —COOH and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is naphthyl substituted with —COOH only. In some embodiments, R⁴ is naphthyl substituted with —COOH and further substituted with one substituent selected from halogen, C₁-C₄ alkyl, C₁-C₄ alkoxy, hydroxy C₁-C₄ alkyl, hydroxyl, and cyanomethyl.

In some embodiments, R⁴ is tetrahydronaphthalenyl. In some embodiments, R⁴ is tetrahydronaphthalenyl substituted with R⁵ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is tetrahydronaphthalenyl substituted with R⁵ only. In some embodiments, R⁴ is tetrahydronaphthalenyl substituted with R⁵ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is tetrahydronaphthalenyl substituted with —C(O)R⁸ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is tetrahydronaphthalenyl substituted with —C(O)R⁸ only. In some embodiments, R⁴ is tetrahydronaphthalenyl substituted with —C(O)R⁸ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is tetrahydronaphthalenyl substituted with —COOH and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R⁴ is tetrahydronaphthalenyl substituted with —COOH only. In some embodiments, R⁴ is tetrahydronaphthalenyl substituted with —COOH and further substituted with one substituent selected from halogen, C₁-C₄ alkyl, C₁-C₄ alkoxy, hydroxy C₁-C₄ alkyl, hydroxyl, and cyanomethyl.

In some embodiments, R⁴ is 5- or 6-membered heteroaryl having 1, 2 or 3 ring heteroatoms independently selected from nitrogen, oxygen and sulfur. In some embodiments, R⁴ is 5- or 6-membered heteroaryl having 1, 2 or 3 ring heteroatoms independently selected from nitrogen, oxygen and sulfur, wherein the heteroaryl is substituted with R⁵ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, the heteroaryl is substituted with R⁵ only. In some embodiments, the heteroaryl is substituted with R⁵ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is 5- or 6-membered heteroaryl having 1, 2 or 3 ring heteroatoms independently selected from nitrogen, oxygen and sulfur, wherein the heteroaryl substituted with —C(O)R⁸ and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, the heteroaryl is substituted with —C(O)R⁸ only. In some embodiments, the heteroaryl substituted with —C(O)R⁸ and further substituted with one substituent selected from halogen, C₁-C₄ alkyl, C₁-C₄ alkoxy, hydroxy C₁-C₄ alkyl, hydroxyl, and cyanomethyl. In some embodiments, R⁴ is 5- or 6-membered heteroaryl having 1, 2 or 3 ring heteroatoms independently selected from nitrogen, oxygen and sulfur, wherein the heteroaryl substituted with —COOH and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, the heteroaryl is substituted with —COOH only. In some embodiments, the heteroaryl substituted with —COOH and further substituted with one substituent selected from halogen, C₁-C₄ alkyl, C₁-C₄ alkoxy, hydroxy C₁-C₄ alkyl, hydroxyl, and cyanomethyl. In some embodiments, the heteroaryl of R⁴ is 5-membered. In some embodiments, it is 6-membered. In some embodiments, it has 1 heteroatom. In some embodiments, it has 1 heteroatom which is nitrogen. In some embodiments, it has 1 heteroatom which is oxygen. In some embodiments, it has 1 heteroatom which is sulfur. In some embodiments, it has 2 or 3 ring heteroatoms. In some embodiments, it has 2 or 3 ring heteroatoms at least one of which is nitrogen. In some embodiments, it is

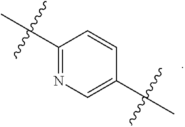 [SMILES: *c1ccc(*)nc1]

In some embodiments, it is bonded to R⁵, e.g., —COOH, at the o-positive relative to the nitrogen atom.

In some embodiments, the further substituent on R⁴ in addition to R⁵ is halogen. In some embodiments, it is —F. In some embodiments, it is —Cl. In some embodiments, it is —Br. In some embodiments, it is —I. In some embodiments, it is C₁-C₄ alkyl. In some embodiments, it is C₁-C₄ alkoxy. In some embodiments, it is hydroxy C₁-C₄ alkyl. In some embodiments, it is hydroxy. In some embodiments, it is cyanomethyl.

In some embodiments, wherein R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R⁵ and further optionally substituted with one or two R⁵′. In some embodiments, R⁴ is phenyl substituted with R⁵ and further optionally substituted with one or two R⁵′. In some embodiments, wherein R⁴ is phenyl para-substituted (unless otherwise indicated, relative to the atom at which R⁴ is attached to the rest of the compound) with R⁵ and further optionally substituted with one or two R⁵′. In some embodiments, R⁴ is phenyl meta-substituted with R⁵ and further optionally substituted with one or two R⁵′. In some embodiments, R⁴ is naphthyl substituted with R⁵ and further optionally substituted with one or two R⁵′. In some embodiments, R⁴ is 1,2,3,4-tetrahydronaphthalenyl substituted with R⁵ and further optionally substituted with one or two R⁵′. In some embodiments, R⁴ is 5-membered heteroaryl substituted with R⁵ and further optionally substituted with one or two R⁵′. In some embodiments, R⁴ is 6-membered heteroaryl substituted with R⁵ and further optionally substituted with one or two R⁵′. In some embodiments, R⁴ is 6-membered heteroaryl para-substituted with R⁵ and further optionally substituted with one or two R⁵′. In some embodiments, R⁴ is 6-membered heteroaryl meta-substituted with R⁵ and further optionally substituted with one or two R⁵′. In some embodiments, a 6-membered heteroaryl is pyridyl. In some embodiments, R⁴ is 9-membered bicyclic heteroaryl substituted with R⁵ and further optionally substituted with one or two R⁵′. In some embodiments, R⁴ is 10-membered bicyclic heteroaryl substituted with R⁵ and further optionally substituted with one or two R^{5′.}

In some embodiments, R⁴ is substituted with one R⁵. In some embodiments, R⁴ is substituted with two R⁵. In some embodiments, a R⁵ is attached to an atom that is boned to an atom to which R⁵ bonds. In some embodiments, a R⁵ is attached to a carbon atom that is boned to an atom to which R⁵ bonds. In some embodiments, a R⁵ is attached to a carbon atom that is boned to a carbon atom to which R⁵ bonds. In some embodiments, R⁴ is or comprises an aromatic ring to which both R⁵ and a R⁵′ are bonded. In some embodiments, R⁵ and a R⁵′ are ortho.

In some embodiments, R⁵′ is halogen. In some embodiments, it is —F. In some embodiments, it is —Cl. In some embodiments, it is —Br. In some embodiments, it is —I.

In some embodiments, R⁵′ is C₁-C₄ alkyl as described herein. In some embodiments, R⁵′ is methyl. In some embodiments, it is ethyl. In some embodiments, R⁵′ is cyclopropyl.

In some embodiments, R⁵′ is C₁-C₄ alkoxy as described herein. In some embodiments, it is methoxy. In some embodiments, it is ethoxy.

In some embodiments, R⁵′ is hydroxy C₁-C₄ alkyl as described herein. In some embodiments, it is —CH₂OH. In some embodiments, it is —CH₂CH₂OH.

In some embodiments, R^{5′} is hydroxyl.

In some embodiments, R⁵′ is R¹⁰ as described herein. In some embodiments, R¹⁰ comprises a nitrogen ring atom. In some embodiments, R¹⁰ is bonded to R⁴ at a nitrogen ring atom.

In some embodiments, R⁵′ is —OR^{6′} wherein R⁶′ is as described herein. In some embodiments, it is —OR¹⁰ wherein R¹⁰ is as described herein.

In some embodiments, R⁵′ is halo C₁-C₆ alkyl. In some embodiments, R⁵′ is C₁-C₆ alkyl substituted with one or more —F.

In some embodiments, R⁵′ is cyano. In some embodiments, R⁵′ is cyanomethyl.

In some embodiments, two R^{5′} are taken together to form an optionally substituted ring as described herein. In some embodiments, two R^{5′} are taken together to form an optionally substituted 4-8 membered partially unsaturated ring as described herein. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, two R^{5′} are taken together to form an optionally substituted 5-6 membered aromatic ring as described herein. In some embodiments, two R^{5′} are taken together to form an optionally substituted 5-membered aromatic ring as described herein. In some embodiments, two R⁵ are taken together to form an optionally substituted 6-membered aromatic ring as described herein. In some embodiments, a formed ring in unsubstituted. In some embodiments, a formed ring is substituted. In some embodiments, a formed ring is substituted with oxo. In some embodiments, a formed ring has a nitrogen ring atom. In some embodiments, a formed ring has an oxygen ring atom. In some embodiments, a formed ring has a nitrogen ring atom and an oxygen ring atom. In some embodiments, a formed ring has two oxygen ring atoms.

In some embodiments, R⁵′ is an amino group or a derivative thereof. In some embodiments, such R⁵′ provide improved activities and/or properties. In some embodiments, R⁵′ is —N(R⁵¹)₂ wherein each R⁵¹ is independently as described herein. In some embodiments, R⁵′ is —NHR⁵¹ wherein R⁵¹ is as described herein.

In some embodiments, R⁵′ is —NHC(O)CH₃. In some embodiments, R⁵′ is —NHC(O)CH₂CH₃. In some embodiments, R⁵¹ is —SO₂—C₁-C₆ aliphatic, wherein the C₁-C₆ aliphatic is optionally substituted. In some embodiments, R⁵¹ is —SO₂—C₁-C₆ aliphatic. In some embodiments, R⁵¹ is —SO₂—C₁-C₆ alkyl. In some embodiments, R⁵′ is —NHS(O)₂CH₃. In some embodiments, R⁵′ is —NH₂. In some embodiments, R⁵′ is —NH(CH₃). In some embodiments, R⁵′ is —NH(CH₂CH₃).

As described herein, each R⁵¹ is independently R⁶′ as described herein.

In some embodiments, each R⁵¹ is independently hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.

In some embodiments, R⁵¹ is hydrogen. In some embodiments, R⁵¹ is optionally substituted C₁-C₆ aliphatic. In some embodiments, R⁵¹ is C₁-C₆ aliphatic. In some embodiments, R⁵¹ is C₁-C₆ alkyl. In some embodiments, R⁵¹ is cyclopropyl. In some embodiments, R⁵¹ is methyl. In some embodiments, R⁵¹ is ethyl. In some embodiments, R⁵¹ is cyclobutyl. In some embodiments, R⁵¹ is —CH₂-cyclopropyl. In some embodiments, R⁵¹ is substituted C₁-C₆ aliphatic. In some embodiments, R⁵¹ is substituted C₁-C₆ alkyl. In some embodiments, R⁵¹ is (S)—CH(CH₃)(CF₃). In some embodiments, R⁵¹ is —CH₂CF₃. In some embodiments, R⁵¹ is substituted C₃-C₆ cycloalkyl. In some embodiments, R⁵¹ is 3,3-difluorocyclobutyl. In some embodiments, R⁵¹ is R¹⁰ as described herein. In some embodiments, R⁵¹ is —CH₂—R¹⁰ wherein R¹⁰ is as described herein.

In some embodiments, R⁵¹ is —C(O)—C₁-C₆ aliphatic, wherein the C₁-C₆ aliphatic is optionally substituted. In some embodiments, R⁵¹ is —C(O)—C₁-C₆ aliphatic. In some embodiments, R⁵¹ is —C(O)—C₁-C₆ alkyl.

In some embodiments, R^{4′} is hydrogen. In some embodiments, R^{4′} is C₁-C₄ alkyl. In some embodiments, R⁴ is hydroxy C₁-C₄ alkyl. In some embodiments, R^{4′} and one R⁶ are taken together to form C₂-C₄ alkylene. In some embodiments, the carbon to which R^{4′} is attached has a configuration of

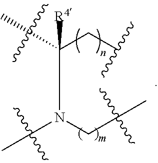

In some embodiments, the carbon to which R^{4′} is attached has a configuration of

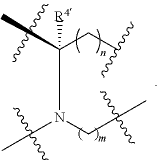

In some embodiments, the carbon to which R^{4′} is attached has a structure of

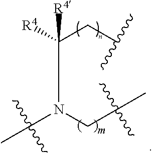

In some embodiments, the carbon to which R^{4′} is attached has a structure of

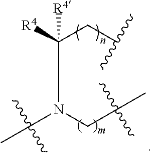

In some embodiments, R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom.

In some embodiments, R⁵ is —COOH or a bioisostere thereof.

In some embodiments, R⁵ is —C(O)R⁸. In some embodiments, R⁵ is —C(O)OH. In some embodiments, R⁵ is —C(O)R⁸, wherein R⁸ is C₁-C₄ alkoxy.

In some embodiments, R⁵ is —CH₂C(O)R⁸. In some embodiments, R⁵ is —CH₂C(O)OH. In some embodiments, R⁵ is —CH₂C(O)R⁸, wherein R⁸ is C₁-C₄ alkoxy.

In some embodiments, R⁵ is R⁹. In some embodiments, R⁵ is —C(O)NHSO₂C₁-C₄alkyl. In some embodiments, R⁵ is —C(O)NHSO₂CH₃. In some embodiments, R⁵ is —C(O)NHSO₂ (cyclopropyl). In some embodiments, R⁵ is —SO₂NHC(O)C₁-C₄alkyl. In some embodiments, R⁵ is —SO₂N(H)ₚ(C₁-C₄alkyl)₂₋ₚ. In some embodiments, R⁵ is —SO₂N(C₁-C₄alkyl)₂. In some embodiments, R⁵ is —SO₂NH(C₁-C₄alkyl). In some embodiments, R⁵ is —SO₂NH₂. In some embodiments, R⁵ is —SO(NH)C₁-C₄alkyl. In some embodiments, R⁵ is —SO₂C₁-C₄alkyl. In some embodiments, R⁵ is cyano. In some embodiments, R⁵ is halogen. In some embodiments, R⁵ is —F. In some embodiments, R⁵ is —Cl. In some embodiments, R⁵ is —Br. In some embodiments, R⁵ is —I. In some embodiments, R⁵ is hydroxy C₁-C₄ alkyl.

In some embodiments, R⁵ is —B(R⁸)₂. In some embodiments, R⁵ is —B(R⁸)₂, wherein each R⁸ is independently hydroxy or C₁-C₄ alkoxy. In some embodiments, R⁵ is —B(OH)₂. In some embodiments, R⁵ is —B(R⁸)₂, wherein each R⁸ is independently C₁-C₄ alkoxy.

In some embodiments, R⁵ is 5-membered heteroaryl having 1-4 ring nitrogen atoms and 0 or 1 ring oxygen or sulfur atom. In some embodiments, R⁵ is 5-membered heteroaryl having 1-4 ring nitrogen atoms. In some embodiments, R⁵ is tetrazolyl.

In some embodiments, each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy.

In some embodiments, R⁶ is hydrogen. In some embodiments, R⁶ is not hydrogen. In some embodiments, R⁶ is halogen. In some embodiments, R⁶ is —F. In some embodiments, R⁶ is —Cl. In some embodiments, R⁶ is —Br. In some embodiments, R⁶ is —I. In some embodiments, R⁶ is hydroxy. In some embodiments, R⁶ is amino. In some embodiments, R⁶ is mono- and di-C₁-C₆ alkylamino. In some embodiments, R⁶ is C₁-C₆ alkyl.

In some embodiments, R⁶ is halo C₁-C₆ alkyl. In some embodiments, R⁶ is halo C₁-C₄ alkyl. In some embodiments, R⁶ is —CF₃. In some embodiments, R⁶ is —CHF₂. In some embodiments, R⁶ is —CH₂F.

In some embodiments, R⁶ is hydroxy C₁-C₆ alkyl. In some embodiments, R⁶ is hydroxy C₁-C₄ alkyl. In some embodiments, R⁶ is —CH₂OH.

In some embodiments, R⁶ is cyano C₁-C₆ alkyl. In some embodiments, R⁶ is cyano C₁-C₄ alkyl. In some embodiments, R⁶ is C₁-C₆ alkoxy. In some embodiments, R⁶ is C₁-C₄ alkoxy.

In some embodiments, two R⁶ on a carbon atom are taken together to form ═O. In some embodiments, —O is on a carbon at the o-position relative to the carbon to which R⁴ and R^{4′} are attached. In some embodiments, ═O is on a carbon at the p-position relative to the carbon to which R⁴ and R^{4′} are attached. In some embodiments, two R⁶ on a carbon atom are taken together with the carbon atom to form a 3-membered monocyclic saturated ring having 0-1 ring atoms independently selected from nitrogen, sulfur and oxygen. In some embodiments, a formed ring is

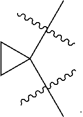 [SMILES: *C1(*)CC1]

In some embodiments, a formed ring is

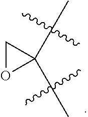 [SMILES: *C1(*)CO1]

In some embodiments, two R⁶ on a carbon atom are taken together with the carbon atom to form a 3-membered monocyclic saturated ring having a nitrogen ring atom. In some embodiments, two R⁶ on a carbon atom are taken together with the carbon atom to form a 3-membered monocyclic saturated ring having a sulfur ring atom.

In some embodiments, two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is saturated. In some embodiments, a formed ring is partially unsaturated. In some embodiments, a formed ring has no heteroatom ring atom. In some embodiments, a formed ring has 1-3 ring atoms independently selected from nitrogen, sulfur and oxygen. In some embodiments, one ring atom is a heteroatom. In some embodiments, two ring atoms are independently a heteroatom. In some embodiments, three ring atoms are independently a heteroatom. In some embodiments, a ring atom is nitrogen. In some embodiments, a ring atom is sulfur. In some embodiments, a ring atom is oxygen. In some embodiments, a formed ring is substituted. In some embodiments, a formed ring is unsubstituted. In some embodiments, a substituent is halogen. In some embodiments, a substituent is C₁-C₄ alkyl. In some embodiments, a substituent is halo C₁-C₄ alkyl. In some embodiments, a substituent is C₁-C₄ alkoxy. In some embodiments, a substituent is hydroxy. In some embodiments, a substituent is oxo.

In some embodiments, t is 0. In some embodiments, t is 1. In some embodiments, t is 3. In some embodiments, t is 4. In some embodiments, t is 1, 2, 3 or 4, and each R⁶ is not hydrogen. In some embodiments, t is 1, 2, 3, or 4, and each R⁶ is independently halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy.

In some embodiments, R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic, C₃-C₆ cycloaliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.

In some embodiments, R^{6′} is hydrogen. In some embodiments, R^{6′} is not hydrogen.

In some embodiments, R^{6′} is C₁-C₆ aliphatic optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.

In some embodiments, R⁶′ is optionally substituted C₁₋₆ aliphatic. In some embodiments, R⁶′ is C₁₋₆ aliphatic. In some embodiments, R⁶′ is C₁-C₆ alkyl optionally substituted as described herein. In some embodiments, R^{6′} is C₁-C₆ alkyl. In some embodiments, R⁶′ is C₁. In some embodiments, R⁶′ is C₂. In some embodiments, R⁶′ is C3. In some embodiments, R⁶′ is C₄. In some embodiments, R⁶′ is C₅. In some embodiments, R⁶′ is C₆. In some embodiments, R⁶′ is C₁-C₅. In some embodiments, R⁶′ is C₁-C₄. In some embodiments, R⁶′ is C₁-C₃. In some embodiments, R⁶′ is C₁-C₂. In some embodiments, R⁶′ is C₂-C₆. In some embodiments, R⁶′ is C₂-C₅. In some embodiments, R⁶′ is C₂-C₄. In some embodiments, R⁶′ is C₂-C₃. In some embodiments, R⁶′ is C₃-C₆. In some embodiments, R⁶′ is methyl. In some embodiments, R⁶′ is ethyl. In some embodiments, R⁶′ is n-propyl. In some embodiments, R⁶′ is isopropyl. In some embodiments, R⁶′ is —CH₂-cyclopropyl. In some embodiments, R⁶′ is —CH₂-cyclobutyl. In some embodiments, R⁶′ is —CH₂-cyclopentyl. In some embodiments, R⁶′ is 2-methylpropyl. In some embodiments, R⁶′ is 1-methylpropyl. In some embodiments, R⁶′ is n-butyl. In some embodiments, R⁶′ is —CH₂CH(CH₃)CH₂CH₃. In some embodiments, R⁶′ is —CH₂CH₂CH(CH₃)₂.

In some embodiments, R⁶′ is C₃₋₆ (C₃, C₄, C₅ or C₆) cycloaliphatic optionally substituted as described herein. In some embodiments, R⁶′ is C₃-C₆ (C₃, C₄, C₅ or C₆) cycloalkyl optionally substituted as described herein. In some embodiments, R⁶′ is C₃₋₆ (C₃, C₄, C₅ or C₆) cycloaliphatic. In some embodiments, R⁶′ is C₃-C₆ (C₃, C₄, C₅ or C₆) cycloalkyl. In some embodiments, cycloalkyl is cyclopropyl. In some embodiments, it is cyclobutyl. In some embodiments, it is cyclopentyl. In some embodiments, it is cyclohexyl.

In some embodiments, R⁶′ is C₂-C₆ alkenyl optionally substituted as described herein. In some embodiments, R⁶′ is C₂-C₆ alkenyl. In some embodiments, an alkenyl is a terminal alkenyl group. In some embodiments, R⁶′ is —CH₂CH═CH₂.

In some embodiments, R⁶′ is C₂-C₆ alkynyl optionally substituted as described herein. In some embodiments, R⁶′ is C₂-C₆ alkynyl. In some embodiments, an alkynyl is a terminal alkynyl group. In some embodiments, R⁶′ is —CH₂C≡CH.

In some embodiments, R⁶′ is —CH₂-(hydroxy C₁-C₄ alkyl). In some embodiments, R^{6′} is —CH₂CH₂OH.

In some embodiments, a group (e.g., C₁₋₆ aliphatic, C₃-C₆ cycloaliphatic, phenyl, etc.) of R^{6′} is substituted as described herein. In some embodiments, it is substituted with halogen (e.g., —F, —Cl, —Br or —I). In some embodiments, it is substituted with —F. For example, in some embodiments, R^{6′} is C₁₋₆ aliphatic substituted with one or more —F. In some embodiments, R^{6′} is C₁₋₆ alkyl substituted with one or more —F. In some embodiments, R^{6′} is —(CH₂)₂—F. In some embodiments, R^{6′} is —(CH₂)₃—F. In some embodiments, R^{6′} is —CH₂—CHF₂. In some embodiments, R^{6′} is —CH(CH₃)—CHF₂. In some embodiments, R^{6′} is —CH₂—CF₃. In some embodiments, R^{6′} is —CH₂—CH₂—CHF₂. In some embodiments, R^{6′} is —CH₂—CH₂—CF₃. In some embodiments, R^{6′} is —CH₂—CF₂—CH₃. In some embodiments, R^{6′} is —CH(CH₃)—CH₂—CF₃. In some embodiments, R^{6′} is C₃-C₆ substituted one or more —F. In some embodiments, R^{6′} is cyclopropyl substituted with —F.

In some embodiments, a group (e.g., C₁₋₆ aliphatic, C₃-C₆ cycloaliphatic, phenyl, etc.) of R⁶ is substituted as described herein. In some embodiments, it is substituted with C₁-C₄ (e.g., C₁, C₂, C₃ or C₄)alkoxy. In some embodiments, it is substituted with CH₃O—. In some embodiments, R^{6′} is —CH₂CH₂OCH₃.

In some embodiments, R⁶′ is R¹⁰. In some embodiments, R^{6′} is 3-6 membered heterocyclyl optionally substituted as described herein. In some embodiments, R^{6′} is 3-membered heterocyclyl optionally substituted as described herein. In some embodiments, R^{6′} is 4-membered heterocyclyl optionally substituted as described herein. In some embodiments, R^{6′} is 5-membered heterocyclyl optionally substituted as described herein. In some embodiments, R^{6′} is 6-membered heterocyclyl optionally substituted as described herein. In some embodiments, a heterocyclyl group is substituted. In some embodiments, it is unsubstituted. In some embodiments, a heterocyclyl group has a ring oxygen atom. In some embodiments, R^{6′} is

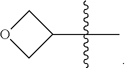 [SMILES: *C1COC1]

In some embodiments, R^{6′} is 5-6 membered heteroaryl optionally substituted as described herein. In some embodiments, R^{6′} is 5-membered heteroaryl optionally substituted as described herein. In some embodiments, R^{6′} is 6-membered heteroaryl optionally substituted as described herein. In some embodiments, a heteroaryl group is substituted. In some embodiments, a heteroaryl group is unsubstituted. In some embodiments, R⁶′ is

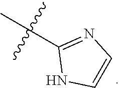 [SMILES: *c1ncc[nH]1]

In some embodiments, R⁶′ is

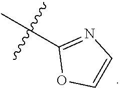 [SMILES: *c1ncco1]

In some embodiments, R⁶′ is

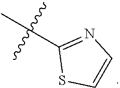 [SMILES: *c1nccs1]

In some embodiments, R⁶′ is

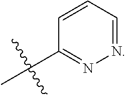 [SMILES: *c1cccnn1]

In some embodiments, R⁶′ is

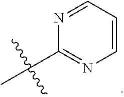 [SMILES: *c1ncccn1]

In some embodiments, the atom of R¹⁰ that forms a bond with the nitrogen to which R¹⁰ is attached is a carbon atom.

In some embodiments, R⁶′ is —CH₂—C₃-C₆ cycloaliphatic wherein the cycloaliphatic group is optionally substituted as described herein. In some embodiments, a cycloaliphatic group is a cycloalkyl group. In some embodiments, R⁶′ is —CH₂—C₃-C₆ cycloalkyl wherein the cycloaliphatic group is optionally substituted as described herein. In some embodiments, R⁶′ is —CH₂—C₃-C₆ cycloaliphatic. In some embodiments, R⁶′ is —CH₂—C₃-C₆ cycloalkyl. In some embodiments, a cycloalkyl is cyclopropyl. In some embodiments, a cycloalkyl is cyclobutyl. In some embodiments, a cycloalkyl is cyclopentyl. In some embodiments, a cycloalkyl is cyclohexyl.

In some embodiments, R⁶′ is —CH₂—R¹⁰, wherein R¹⁰ is as described herein.

In some embodiments, R⁶′ is —CH₂-(hydroxy C₁-C₄ alkyl). In some embodiments, R^{6′} is —CH₂CH₂OH.

In some embodiments, R⁶′ is phenyl optionally substituted as described herein. In some embodiments, a phenyl group is substituted. In some embodiments, a phenyl group is substituted with one or more halogen. In some embodiments, a phenyl group is unsubstituted. In some embodiments, R⁶′ is phenyl.

In some embodiments, R^{6′} is —C(O)—C₁-C₆ aliphatic, wherein the C₁-C₆ aliphatic is as described herein. In some embodiments, R⁶′ is —C(O)—C₁-C₆ aliphatic, wherein the C₁-C₆ aliphatic is optionally substituted as described herein. In some embodiments, R^{6′} is —C(O)—C₁-C₆ alkyl. In some embodiments, R^{6′} is —C(O)—CH₃.

In some embodiments, R⁶′ is —SO₂—C₁-C₆ aliphatic, wherein the C₁-C₆ aliphatic is as described herein. In some embodiments, R⁶′ is —SO₂—C₁-C₆ aliphatic, wherein the C₁-C₆ aliphatic is optionally substituted as described herein. In some embodiments, R^{6′} is —SO₂—C₁-C₆ alkyl. In some embodiments, R⁶′ is —SO₂—CH₃.

In some embodiments, R⁶′ is —CH₂-phenyl, wherein the phenyl group is optionally substituted as described herein. In some embodiments, a phenyl group is substituted. In some embodiments, a phenyl group is substituted with one or more halogen. In some embodiments, a phenyl group is unsubstituted. In some embodiments, R⁶′ is —CH₂-phenyl.

In some embodiments, R⁶′ is —CH₂-(amino C₁-C₄ alkyl).

In some embodiments, R⁶′ is —CH₂-(mono- and di-C₁-C₄ alkylamino C₁-C₄ alkyl).

In some embodiments, R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.

In some embodiments, R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered.

In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is monocyclic. In some embodiments, a formed ring a bicyclic. In some embodiments, a formed ring is saturated. In some embodiments, a formed ring is partially saturated. In some embodiments, a formed ring is aromatic. In some embodiments, a formed ring is 5-membered and is aromatic. In some embodiments, a formed ring is 6-membered and is aromatic. In some embodiments, a formed ring has no additional ring heteroatom in addition to the nitrogen atom to which R⁶′ is attached. In some embodiments, a formed ring has 1 or 2 additional ring heteroatoms in addition to the nitrogen atom to which R⁶′ is attached. In some embodiments, an additional ring heteroatom is nitrogen. In some embodiments, an additional ring heteroatom is oxygen.

In some embodiments, R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 3-8 membered monocyclic or bicyclic saturated, or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 3-8 membered monocyclic or bicyclic saturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 3-8 membered monocyclic or bicyclic partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 5-6 membered monocyclic or bicyclic aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 5-8 membered monocyclic saturated or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 5-8 membered monocyclic saturated or partially unsaturated ring having 1 ring nitrogen atom. In some embodiments, R⁶ and R^{6′} are taken together with their intervening atoms to form a 5-8 membered monocyclic saturated ring having 1 ring nitrogen atom. In some embodiments, R⁶ and R^{6′} are taken together with their intervening atoms to form an optionally substituted 5-membered monocyclic saturated ring having 1 ring nitrogen atom. In some embodiments, R⁶ and R^{6′} are taken together with their intervening atoms to form a 5-membered monocyclic saturated ring having 1 ring nitrogen atom, wherein the ring is substituted with one or more substituents each of which is independently halogen. In some embodiments, R⁶ and R^{6′} are taken together with their intervening atoms to form an optionally substituted 4-membered monocyclic saturated ring having 1 ring nitrogen atom. In some embodiments, R⁶ and R^{6′} are taken together with their intervening atoms to form an optionally substituted 6-membered monocyclic saturated ring having 1 ring nitrogen atom. In some embodiments, R⁶ and R^{6′} are taken together with their intervening atoms to form an optionally substituted 6-membered monocyclic saturated ring having 1 ring nitrogen atom and 1 ring oxygen atom. In some embodiments, R⁶ and R^{6′} are taken together with their intervening atoms to form a 6-8 membered bicyclic saturated or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo. In some embodiments, R⁶ and R^{6′} are taken together with their intervening atoms to form a 6-membered bicyclic saturated or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.

In some embodiments, R⁶ and R^{6′} are taken together with their intervening atoms to form optionally substituted

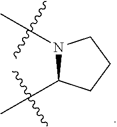 [SMILES: *[C@@H]1CCCN1*]

In some embodiments, R⁶ and R^{6′} are taken together with their intervening atoms to form optionally substituted

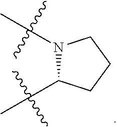 [SMILES: *[C@H]1CCCN1*]

In some embodiments, R⁶ and R^{6′} are taken together with their intervening atoms to form

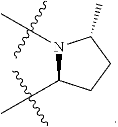 [SMILES: *[C@@H]1CC[C@@H](C)N1*]

In some embodiments, R⁶ and R^{6′} are taken together with their intervening atoms to form

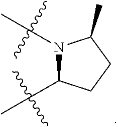 [SMILES: *[C@@H]1CC[C@H](C)N1*]

In some embodiments, R⁶ and R^{6′} are taken together with their intervening atoms to form

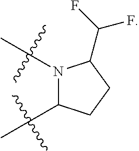 [SMILES: *C1CCC(C(F)F)N1*]

In some embodiments, R⁶ and R^{6′} are taken together with their intervening atoms to form optionally substituted

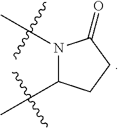 [SMILES: *C1CCC(=O)N1*]

In some embodiments, R⁶ and R^{6′} are taken together with their intervening atoms to form

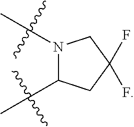 [SMILES: *C1CC(F)(F)CN1*]

In some embodiments, R⁶ and R^{6′} are taken together with their intervening atoms to form

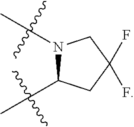 [SMILES: *[C@@H]1CC(F)(F)CN1*]

In some embodiments, R⁶ and R^{6′} are taken together with their intervening atoms to form

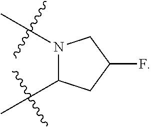 [SMILES: *C1CC(F)CN1*]

In some embodiments, R⁶ and R^{6′} are taken together with their intervening atoms to form

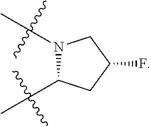 [SMILES: *[C@H]1C[C@@H](F)CN1*]

In some embodiments, R⁶ and R^{6′} are taken together with their intervening atoms to form

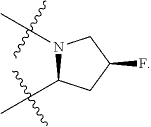 [SMILES: *[C@@H]1C[C@H](F)CN1*]

In some embodiments, R⁶ and R^{6′} are taken together with their intervening atoms to form

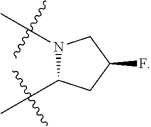 [SMILES: *[C@H]1C[C@H](F)CN1*]

In some embodiments, R⁶ and R^{6′} are taken together with their intervening atoms to form

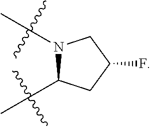 [SMILES: *[C@@H]1C[C@@H](F)CN1*]

In some embodiments, R⁶ and R^{6′} are taken together with their intervening atoms to form optionally substituted

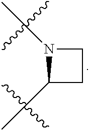 [SMILES: *[C@@H]1CCN1*]

In some embodiments, R⁶ and R^{6′} are taken together with their intervening atoms to form optionally substituted

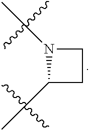 [SMILES: *[C@H]1CCN1*]

In some embodiments, R⁶ and R^{6′} are taken together with their intervening atoms to form optionally substituted

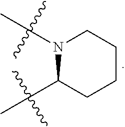 [SMILES: *[C@@H]1CCCCN1*]

In some embodiments, R⁶ and R^{6′} are taken together with their intervening atoms to form optionally substituted

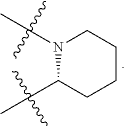 [SMILES: *[C@H]1CCCCN1*]

In some embodiments, R⁶ and R^{6′} are taken together with their intervening atoms to form

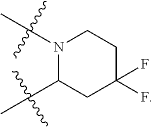 [SMILES: *C1CC(F)(F)CCN1*]

In some embodiments, R⁶ and R^{6′} are taken together with their intervening atoms to form optionally substituted

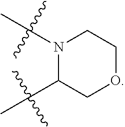 [SMILES: *C1COCCN1*]

In some embodiments, R⁶ and R^{6′} are taken together with their intervening atoms to form optionally substituted

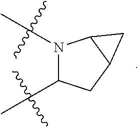 [SMILES: *C1CC2CC2N1*]

In some embodiments, R⁶ and R^{6′} are taken together with their intervening atoms to form optionally substituted

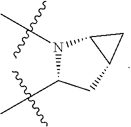 [SMILES: *[C@H]1C[C@H]2C[C@H]2N1*]

In some embodiments, R⁶ and R^{6′} are taken together with their intervening atoms to form optionally substituted

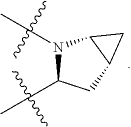 [SMILES: *[C@@H]1C[C@H]2C[C@H]2N1*]

In some embodiments, R⁶ and R^{6′} are taken together with their intervening atoms to form optionally substituted

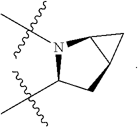 [SMILES: *[C@@H]1C[C@@H]2C[C@@H]2N1*]

In some embodiments, R⁷ is hydroxy. In some embodiments, R⁷ is C₁-C₄ alkoxy. In some embodiments, R⁷ is amino. In some embodiments, R⁷ is mono-C₁-C₄ alkylamino. In some embodiments, R⁷ is di-C₁-C₄ alkylamino.

In some embodiments, each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or R⁸ is mono- and di-C₁-C₄ alkylamino which is optionally substituted with one or more substituents independently selected from halogen, hydroxy and C₁-C₄ alkyl.

In some embodiments, R⁸—OH. In some embodiments, R⁸ is C₁₋₄ alkoxy. In some embodiments, R⁸ is methoxy. In some embodiments, R⁸ is ethoxy. In some embodiments, R⁸ is amino. In some embodiments, R⁸ is 5-, 6- or 7-membered saturated heterocyclyl having 1, 2, or 3 ring heteroatoms independently selected from nitrogen, oxygen and sulfur. In some embodiments, R⁸ is mono-C₁-C₄ alkylamino which is optionally substituted with one or more substituents independently selected from halogen, hydroxy and C₁-C₄ alkyl. In some embodiments, R⁸ is mono-C₁-C₄ alkylamino. In some embodiments, R⁸ is di-C₁-C₄ alkylamino which is optionally substituted with one or more substituents independently selected from halogen, hydroxy and C₁-C₄ alkyl. In some embodiments, R⁸ is di-C₁-C₄ alkylamino.

In some embodiments, R⁸ is 5-7 membered saturated heterocyclyl having 1, 2, or 3 ring heteroatoms independently selected from nitrogen, oxygen and sulfur. In some embodiments, R⁸ is mono- and di-C₁-C₄ alkylamino which is optionally substituted with one or more substituents independently selected from halogen, hydroxy and C₁-C₄ alkyl.

In some embodiments, R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups. In some embodiments, R⁹ is a 5-membered heteroaryl having 1 to 4 ring nitrogen atoms and 0 ring oxygen or sulfur atom, which heteroaryl is optionally substituted with 0 to 2 C₁-C₄ alkyl groups. In some embodiments, a heteroaryl group is substituted. In some embodiments, a heteroaryl group is unsubstituted.

In some embodiments, R¹⁰ is optionally substituted 3-6 membered heterocyclyl. In some embodiments, R¹⁰ is optionally substituted 3-membered heterocyclyl. In some embodiments, R¹⁰ is optionally substituted 4-membered heterocyclyl. In some embodiments, R¹⁰ is optionally substituted 5-membered heterocyclyl. In some embodiments, R¹⁰ is optionally substituted 6-membered heterocyclyl. In some embodiments, R¹⁰ is optionally substituted 5-membered heteroaryl. In some embodiments, R¹⁰ is optionally substituted 6-membered heteroaryl. In some embodiments, R¹⁰ is substituted. In some embodiments, R¹⁰ is unsubstituted. In some embodiments, R¹⁰ has one and only one ring heteroatom. In some embodiments, a ring heteroatom is nitrogen. In some embodiments, a ring heteroatom is oxygen.

In some embodiments, R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur. In some embodiments, R¹⁰ is 3-, 4-, 5- or 6-membered heterocyclyl optionally substituted as described herein. In some embodiments, R¹⁰ is 5- or 6-membered heteroaryl optionally substituted as described herein. In some embodiments, a heterocyclyl group is substituted. In some embodiments, a heterocyclyl group is unsubstituted. In some embodiments, a heteroaryl group is substituted. In some embodiments, a heteroaryl group is unsubstituted. In some embodiments, at least one ring atom of heterocyclyl is nitrogen. In some embodiments, at least one ring atom of heteroaryl is nitrogen. In some embodiments, each heteroatom ring atom of heterocyclyl is nitrogen. In some embodiments, each heteroatom ring atom of heteroaryl is nitrogen.

In some embodiments, R¹⁰ is optionally substituted 5-6 membered heteroaryl having 1-3 ring atoms independently selected from nitrogen, oxygen and sulfur. In some embodiments, R¹⁰ is 5-membered heteroaryl having 1-3 ring nitrogen atoms substituted with C₁-C₄ alkyl. In some embodiments, R¹⁰ is

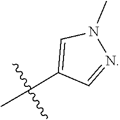 [SMILES: *c1cnn(C)c1]

In some embodiments, R¹⁰ is optionally substituted 3-6 membered heterocyclyl. In some embodiments, R¹⁰ is optionally substituted 6-membered heterocyclyl. In some embodiments, R¹⁰ is

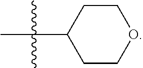 [SMILES: *C1CCOCC1]

In some embodiments, R¹⁰ is

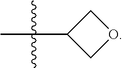 [SMILES: *C1COC1]

In some embodiments, provided compounds comprise enriched levels of one or more isotopes. For example, in some embodiments, provided compounds comprise enriched levels of deuterium (D). In some embodiments, R^{6′} is —CD₃. Various technologies are available for incorporating enriched levels of various isotopes and can be utilized in accordance with the present disclosure. In some embodiments, a compound has an isotopic purity of about 5%-100% (e.g., about 5%, 10%, 20%, 25%, 30%, 40%, 50%, 60%, 70%, 75%, 80%, 85%, 90%, 95%, 96%, 97%, 98%, 99%, 99.5%, 99.9% or more, or about 5%-100%, 10%-100%, 20%-100%, 30%-100%, 50%-100%, 80%-100%, 90-100%, 95%-100%, 96%-100%, 97%-100%, 98%-100%, 99%-100%, 95-99%, 95%-99.5%, 95%-99.9%, etc). For example, in some embodiments, about 5%-100% (e.g., about 5%, 10%, 20%, 25%, 30%, 40%, 50%, 60%, 70%, 75%, 80%, 85%, 90%, 95%, 96%, 97%, 98%, 99%, 99.5%, 99.9% or more, or about 5%-100%, 10%-100%, 20%-100%, 30%-100%, 50%-100%, 80%-100%, 90-100%, 95%-100%, 96%-100%, 97%-100%, 98%-100%, 99%-100%, 95-99%, 95%-99.5%, 95%-99.9%, etc) of all molecules of a compound wherein R^{6′} is —CH₃ has R^{6′} being —CD₃.

In some embodiments, m is 1. In some embodiments, m is 2. In some embodiments, n is 1. In some embodiments, n is 2. In some embodiments, m is 1 and n is 1. In some embodiments, t is 0. In some embodiments, tis 1. In some embodiments, t is 2. In some embodiments, t is 3. In some embodiments, t is 4. In some embodiments, a provided compound has the structure of:

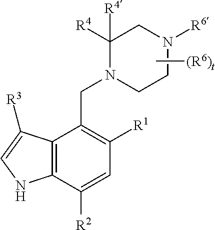

or a pharmaceutically acceptable salt thereof, wherein each variable is independently as described herein. In some embodiments, a provided compound has the structure of:

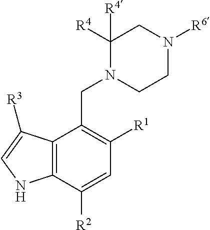

or a pharmaceutically acceptable salt thereof, wherein each variable is independently as described herein. In some embodiments, a provided compound has the structure of:

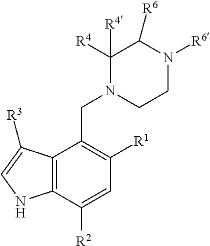

or a pharmaceutically acceptable salt thereof, wherein each variable is independently as described herein. In some embodiments, a provided compound has the structure of:

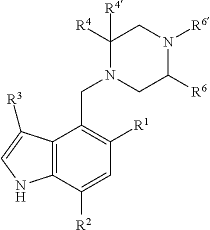

or a pharmaceutically acceptable salt thereof, wherein each variable is independently as described herein. In some embodiments, R⁶ and R^{6′} are taken together with their intervening atoms to form a ring as described herein. In some embodiments, a provided compound has the structure of:

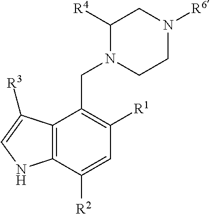

or a pharmaceutically acceptable salt thereof, wherein each variable is independently as described herein.

In some embodiments, a compound is selected from Table C-1 below, or a pharmaceutically acceptable salt thereof. In some embodiments, a composition comprises or delivers a compound selected from C-1 below, or a pharmaceutically acceptable salt thereof. In some embodiments, a compound is selected from Table E-1, or a pharmaceutically acceptable salt thereof. In some embodiments, a composition comprises or delivers a compound selected from E-1, or a pharmaceutically acceptable salt thereof.

TABLE C-1
Certain compounds.

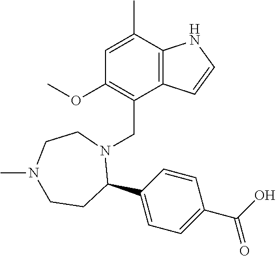 [SMILES: COc1cc(C)c2[nH]ccc2c1CN1CCN(C)CC[C@@H]1c1ccc(C(=O)O)cc1]

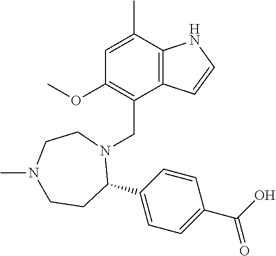 [SMILES: COc1cc(C)c2[nH]ccc2c1CN1CCN(C)CC[C@H]1c1ccc(C(=O)O)cc1]

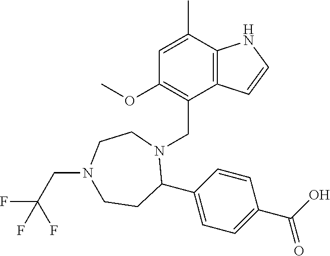 [SMILES: COc1cc(C)c2[nH]ccc2c1CN1CCN(CC(F)(F)F)CCC1c1ccc(C(=O)O)cc1]

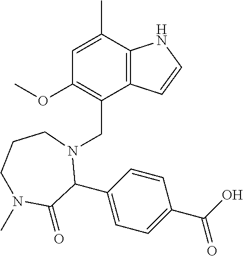 [SMILES: COc1cc(C)c2[nH]ccc2c1CN1CCCN(C)C(=O)C1c1ccc(C(=O)O)cc1]

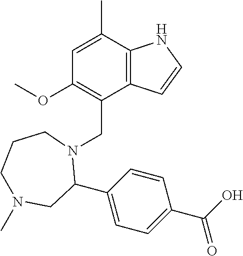 [SMILES: COc1cc(C)c2[nH]ccc2c1CN1CCCN(C)CC1c1ccc(C(=O)O)cc1]

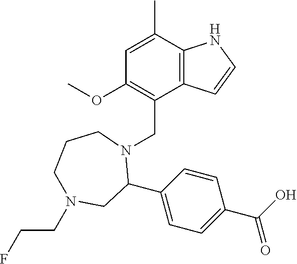 [SMILES: COc1cc(C)c2[nH]ccc2c1CN1CCCN(CCF)CC1c1ccc(C(=O)O)cc1]

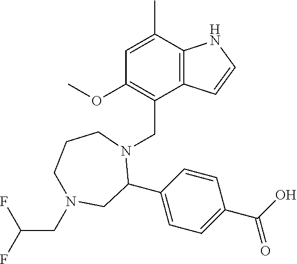 [SMILES: COc1cc(C)c2[nH]ccc2c1CN1CCCN(CC(F)F)CC1c1ccc(C(=O)O)cc1]

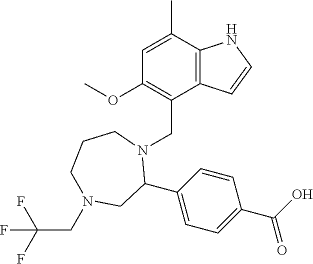 [SMILES: COc1cc(C)c2[nH]ccc2c1CN1CCCN(CC(F)(F)F)CC1c1ccc(C(=O)O)cc1]

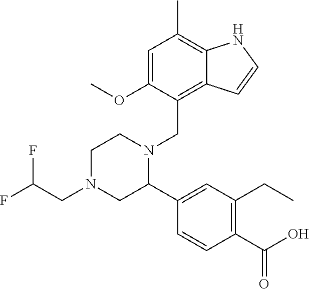 [SMILES: CCc1cc(C2CN(CC(F)F)CCN2Cc2c(OC)cc(C)c3[nH]ccc23)ccc1C(=O)O]

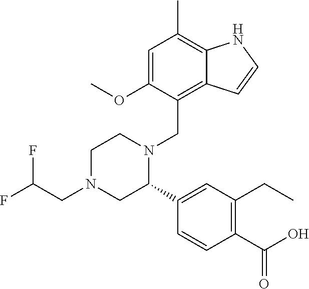 [SMILES: CCc1cc([C@@H]2CN(CC(F)F)CCN2Cc2c(OC)cc(C)c3[nH]ccc23)ccc1C(=O)O]

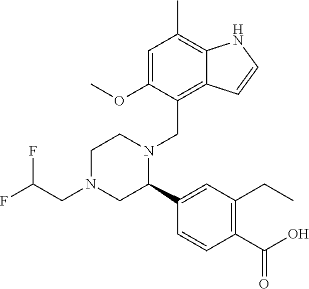 [SMILES: CCc1cc([C@H]2CN(CC(F)F)CCN2Cc2c(OC)cc(C)c3[nH]ccc23)ccc1C(=O)O]

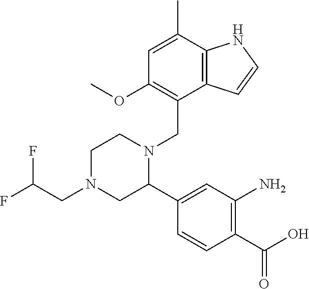 [SMILES: COc1cc(C)c2[nH]ccc2c1CN1CCN(CC(F)F)CC1c1ccc(C(=O)O)c(N)c1]

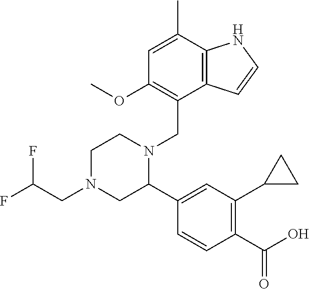 [SMILES: COc1cc(C)c2[nH]ccc2c1CN1CCN(CC(F)F)CC1c1ccc(C(=O)O)c(C2CC2)c1]

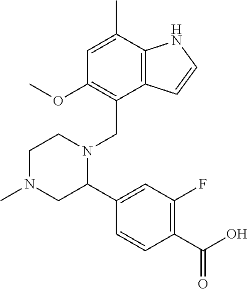 [SMILES: COc1cc(C)c2[nH]ccc2c1CN1CCN(C)CC1c1ccc(C(=O)O)c(F)c1]

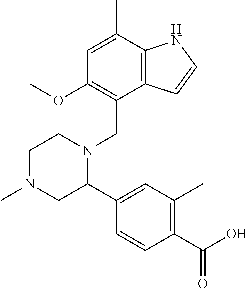 [SMILES: COc1cc(C)c2[nH]ccc2c1CN1CCN(C)CC1c1ccc(C(=O)O)c(C)c1]

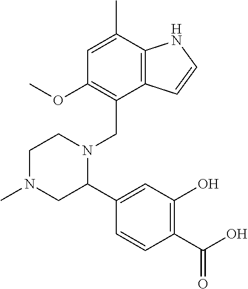 [SMILES: COc1cc(C)c2[nH]ccc2c1CN1CCN(C)CC1c1ccc(C(=O)O)c(O)c1]

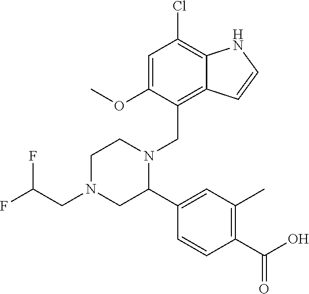 [SMILES: COc1cc(Cl)c2[nH]ccc2c1CN1CCN(CC(F)F)CC1c1ccc(C(=O)O)c(C)c1]

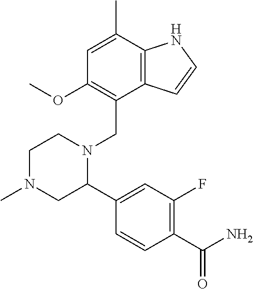 [SMILES: COc1cc(C)c2[nH]ccc2c1CN1CCN(C)CC1c1ccc(C(N)=O)c(F)c1]

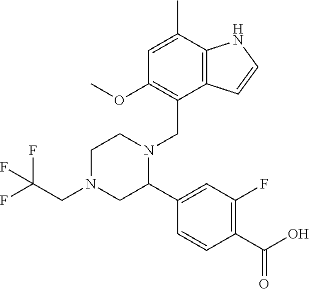 [SMILES: COc1cc(C)c2[nH]ccc2c1CN1CCN(CC(F)(F)F)CC1c1ccc(C(=O)O)c(F)c1]

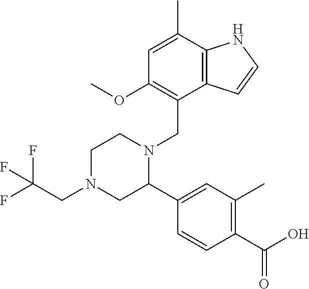 [SMILES: COc1cc(C)c2[nH]ccc2c1CN1CCN(CC(F)(F)F)CC1c1ccc(C(=O)O)c(C)c1]

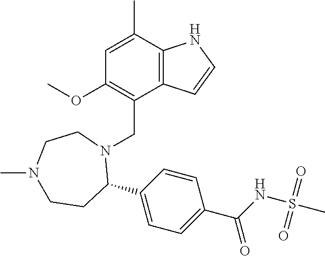

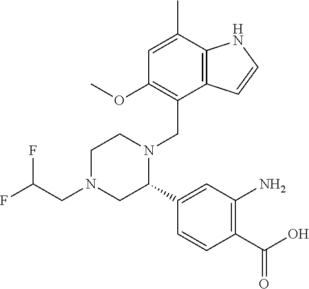 [SMILES: COc1cc(C)c2[nH]ccc2c1CN1CCN(CC(F)F)C[C@H]1c1ccc(C(=O)O)c(N)c1]

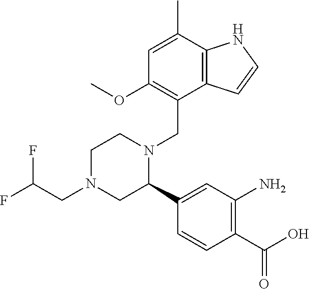 [SMILES: COc1cc(C)c2[nH]ccc2c1CN1CCN(CC(F)F)C[C@@H]1c1ccc(C(=O)O)c(N)c1]

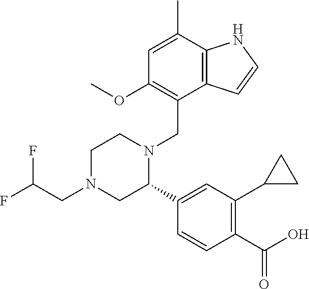 [SMILES: COc1cc(C)c2[nH]ccc2c1CN1CCN(CC(F)F)C[C@H]1c1ccc(C(=O)O)c(C2CC2)c1]

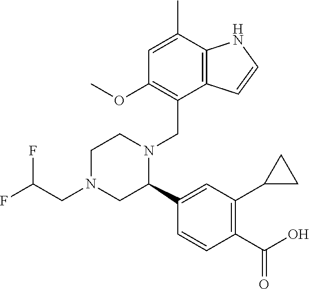 [SMILES: COc1cc(C)c2[nH]ccc2c1CN1CCN(CC(F)F)C[C@@H]1c1ccc(C(=O)O)c(C2CC2)c1]

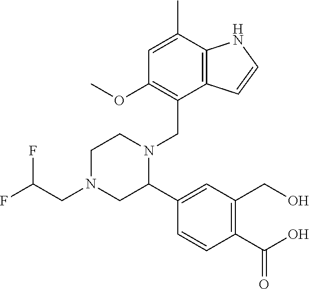 [SMILES: COc1cc(C)c2[nH]ccc2c1CN1CCN(CC(F)F)CC1c1ccc(C(=O)O)c(CO)c1]

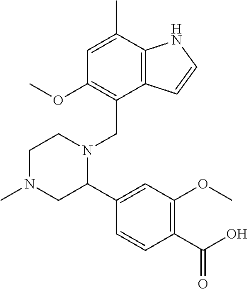

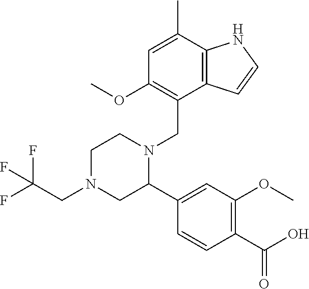 [SMILES: COc1cc(C2CN(CC(F)(F)F)CCN2Cc2c(OC)cc(C)c3[nH]ccc23)ccc1C(=O)O]

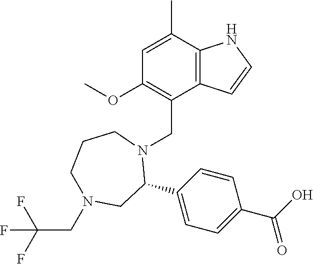 [SMILES: COc1cc(C)c2[nH]ccc2c1CN1CCCN(CC(F)(F)F)C[C@H]1c1ccc(C(=O)O)cc1]

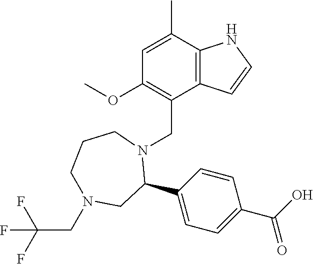 [SMILES: COc1cc(C)c2[nH]ccc2c1CN1CCCN(CC(F)(F)F)C[C@@H]1c1ccc(C(=O)O)cc1]

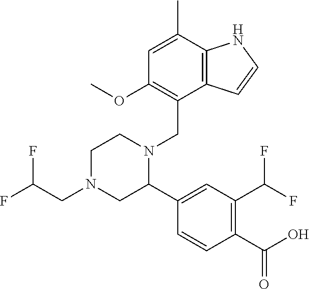 [SMILES: COc1cc(C)c2[nH]ccc2c1CN1CCN(CC(F)F)CC1c1ccc(C(=O)O)c(C(F)F)c1]

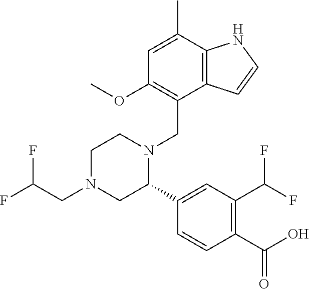 [SMILES: COc1cc(C)c2[nH]ccc2c1CN1CCN(CC(F)F)C[C@H]1c1ccc(C(=O)O)c(C(F)F)c1]

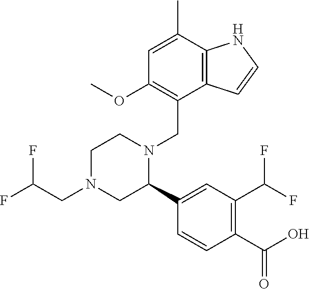 [SMILES: COc1cc(C)c2[nH]ccc2c1CN1CCN(CC(F)F)C[C@@H]1c1ccc(C(=O)O)c(C(F)F)c1]

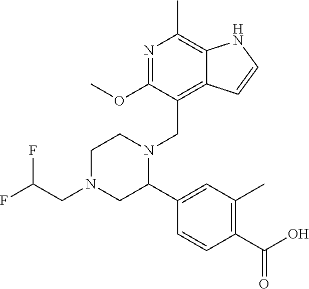 [SMILES: COc1nc(C)c2[nH]ccc2c1CN1CCN(CC(F)F)CC1c1ccc(C(=O)O)c(C)c1]

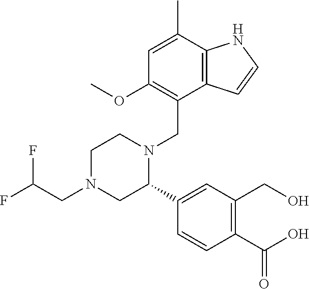 [SMILES: COc1cc(C)c2[nH]ccc2c1CN1CCN(CC(F)F)C[C@H]1c1ccc(C(=O)O)c(CO)c1]

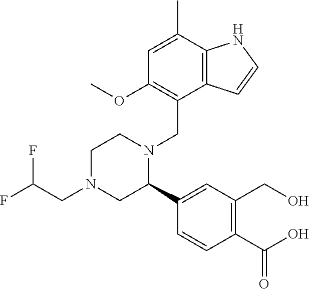 [SMILES: COc1cc(C)c2[nH]ccc2c1CN1CCN(CC(F)F)C[C@@H]1c1ccc(C(=O)O)c(CO)c1]

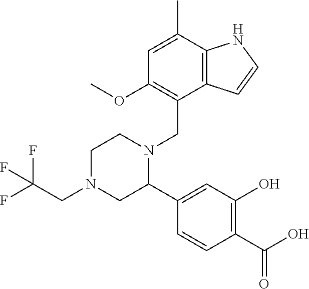 [SMILES: COc1cc(C)c2[nH]ccc2c1CN1CCN(CC(F)(F)F)CC1c1ccc(C(=O)O)c(O)c1]

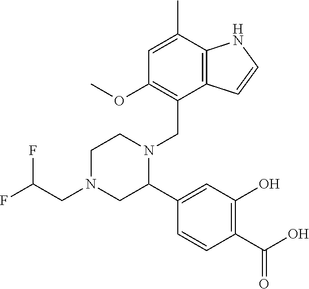 [SMILES: COc1cc(C)c2[nH]ccc2c1CN1CCN(CC(F)F)CC1c1ccc(C(=O)O)c(O)c1]

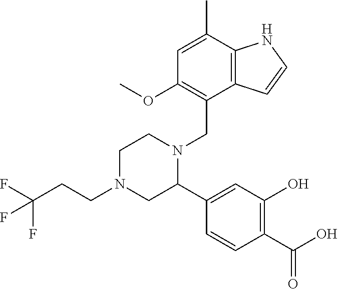 [SMILES: COc1cc(C)c2[nH]ccc2c1CN1CCN(CCC(F)(F)F)CC1c1ccc(C(=O)O)c(O)c1]

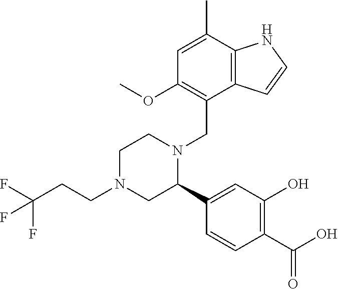 [SMILES: COc1cc(C)c2[nH]ccc2c1CN1CCN(CCC(F)(F)F)C[C@@H]1c1ccc(C(=O)O)c(O)c1]

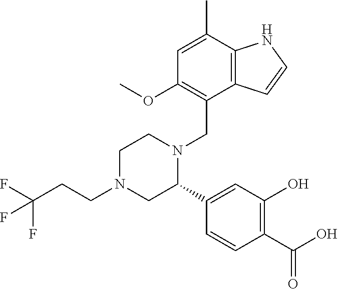 [SMILES: COc1cc(C)c2[nH]ccc2c1CN1CCN(CCC(F)(F)F)C[C@H]1c1ccc(C(=O)O)c(O)c1]

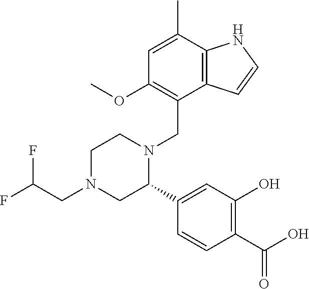 [SMILES: COc1cc(C)c2[nH]ccc2c1CN1CCN(CC(F)F)C[C@H]1c1ccc(C(=O)O)c(O)c1]

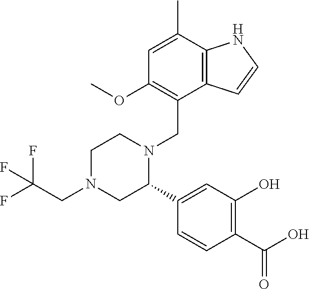 [SMILES: COc1cc(C)c2[nH]ccc2c1CN1CCN(CC(F)(F)F)C[C@H]1c1ccc(C(=O)O)c(O)c1]

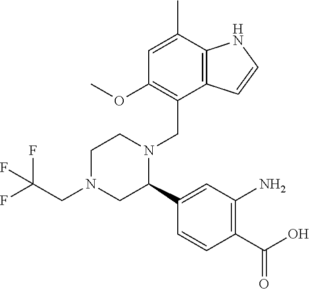 [SMILES: COc1cc(C)c2[nH]ccc2c1CN1CCN(CC(F)(F)F)C[C@@H]1c1ccc(C(=O)O)c(N)c1]

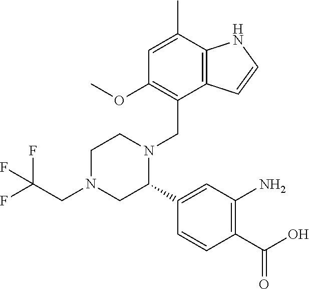 [SMILES: COc1cc(C)c2[nH]ccc2c1CN1CCN(CC(F)(F)F)C[C@H]1c1ccc(C(=O)O)c(N)c1]

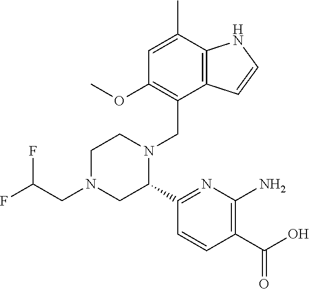 [SMILES: COc1cc(C)c2[nH]ccc2c1CN1CCN(CC(F)F)C[C@H]1c1ccc(C(=O)O)c(N)n1]

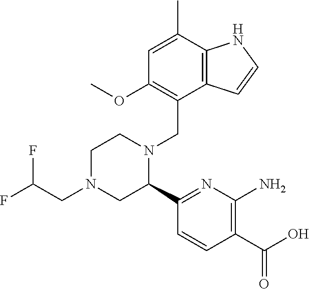 [SMILES: COc1cc(C)c2[nH]ccc2c1CN1CCN(CC(F)F)C[C@@H]1c1ccc(C(=O)O)c(N)n1]

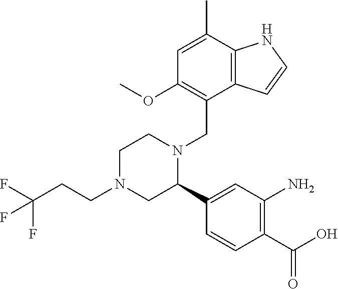 [SMILES: COc1cc(C)c2[nH]ccc2c1CN1CCN(CCC(F)(F)F)C[C@@H]1c1ccc(C(=O)O)c(N)c1]

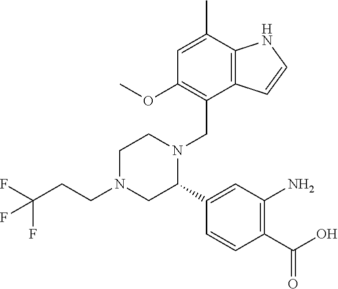 [SMILES: COc1cc(C)c2[nH]ccc2c1CN1CCN(CCC(F)(F)F)C[C@H]1c1ccc(C(=O)O)c(N)c1]

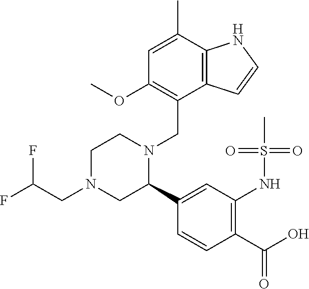 [SMILES: COc1cc(C)c2[nH]ccc2c1CN1CCN(CC(F)F)C[C@@H]1c1ccc(C(=O)O)c(NS(C)(=O)=O)c1]

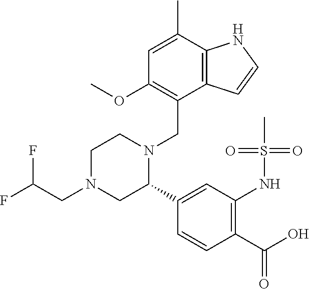 [SMILES: COc1cc(C)c2[nH]ccc2c1CN1CCN(CC(F)F)C[C@H]1c1ccc(C(=O)O)c(NS(C)(=O)=O)c1]

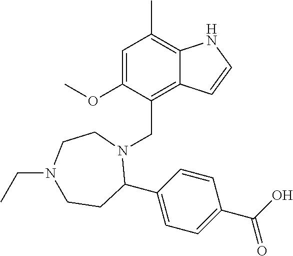 [SMILES: CCN1CCC(c2ccc(C(=O)O)cc2)N(Cc2c(OC)cc(C)c3[nH]ccc23)CC1]

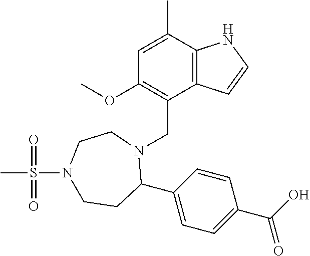 [SMILES: COc1cc(C)c2[nH]ccc2c1CN1CCN(S(C)(=O)=O)CCC1c1ccc(C(=O)O)cc1]

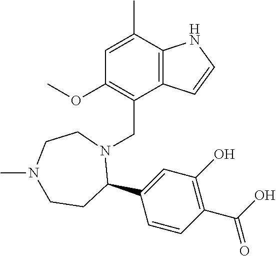 [SMILES: COc1cc(C)c2[nH]ccc2c1CN1CCN(C)CC[C@@H]1c1ccc(C(=O)O)c(O)c1]

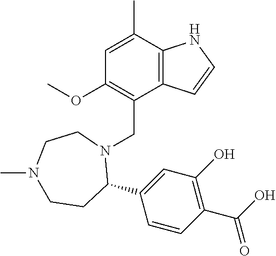 [SMILES: COc1cc(C)c2[nH]ccc2c1CN1CCN(C)CC[C@H]1c1ccc(C(=O)O)c(O)c1]

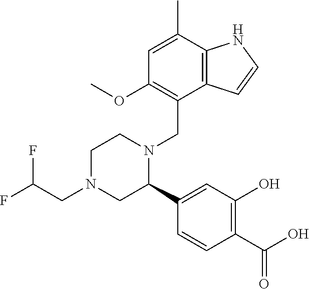 [SMILES: COc1cc(C)c2[nH]ccc2c1CN1CCN(CC(F)F)C[C@@H]1c1ccc(C(=O)O)c(O)c1]

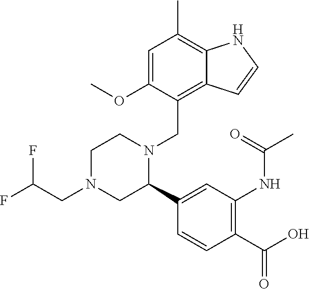 [SMILES: COc1cc(C)c2[nH]ccc2c1CN1CCN(CC(F)F)C[C@@H]1c1ccc(C(=O)O)c(NC(C)=O)c1]

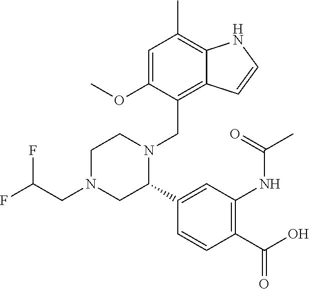 [SMILES: COc1cc(C)c2[nH]ccc2c1CN1CCN(CC(F)F)C[C@H]1c1ccc(C(=O)O)c(NC(C)=O)c1]

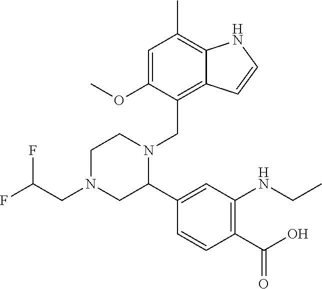 [SMILES: CCNc1cc(C2CN(CC(F)F)CCN2Cc2c(OC)cc(C)c3[nH]ccc23)ccc1C(=O)O]

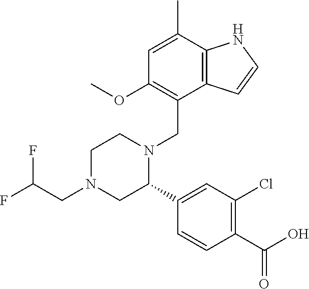 [SMILES: COc1cc(C)c2[nH]ccc2c1CN1CCN(CC(F)F)C[C@H]1c1ccc(C(=O)O)c(Cl)c1]

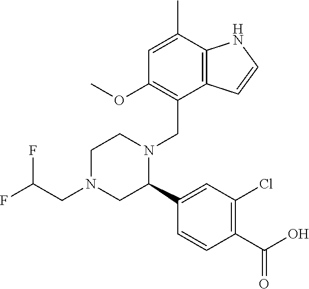 [SMILES: COc1cc(C)c2[nH]ccc2c1CN1CCN(CC(F)F)C[C@@H]1c1ccc(C(=O)O)c(Cl)c1]

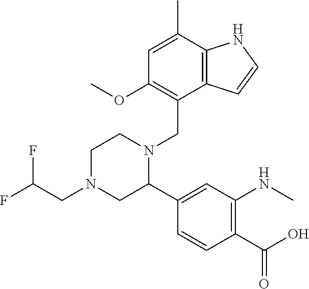 [SMILES: CNc1cc(C2CN(CC(F)F)CCN2Cc2c(OC)cc(C)c3[nH]ccc23)ccc1C(=O)O]

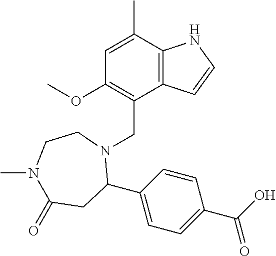 [SMILES: COc1cc(C)c2[nH]ccc2c1CN1CCN(C)C(=O)CC1c1ccc(C(=O)O)cc1]

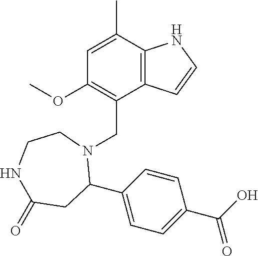 [SMILES: COc1cc(C)c2[nH]ccc2c1CN1CCNC(=O)CC1c1ccc(C(=O)O)cc1]

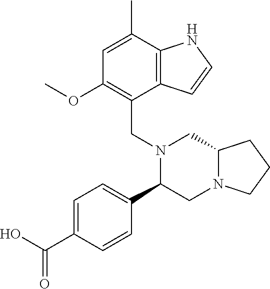 [SMILES: COc1cc(C)c2[nH]ccc2c1CN1C[C@@H]2CCCN2C[C@H]1c1ccc(C(=O)O)cc1]

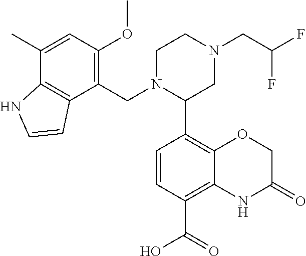 [SMILES: COc1cc(C)c2[nH]ccc2c1CN1CCN(CC(F)F)CC1c1ccc(C(=O)O)c2c1OCC(=O)N2]

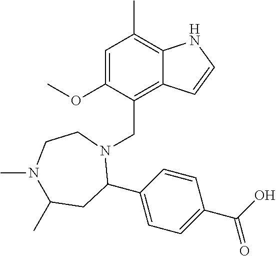 [SMILES: COc1cc(C)c2[nH]ccc2c1CN1CCN(C)C(C)CC1c1ccc(C(=O)O)cc1]

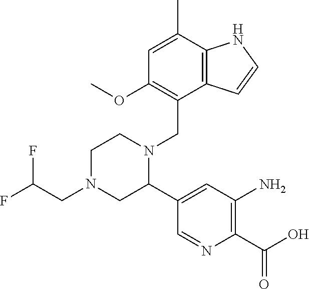 [SMILES: COc1cc(C)c2[nH]ccc2c1CN1CCN(CC(F)F)CC1c1cnc(C(=O)O)c(N)c1]

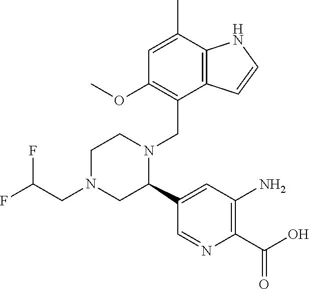 [SMILES: COc1cc(C)c2[nH]ccc2c1CN1CCN(CC(F)F)C[C@@H]1c1cnc(C(=O)O)c(N)c1]

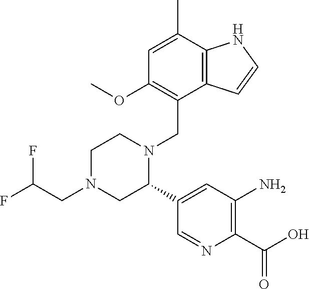 [SMILES: COc1cc(C)c2[nH]ccc2c1CN1CCN(CC(F)F)C[C@H]1c1cnc(C(=O)O)c(N)c1]

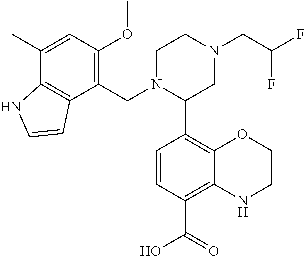 [SMILES: COc1cc(C)c2[nH]ccc2c1CN1CCN(CC(F)F)CC1c1ccc(C(=O)O)c2c1OCCN2]

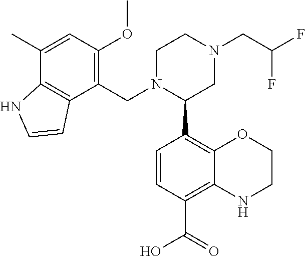 [SMILES: COc1cc(C)c2[nH]ccc2c1CN1CCN(CC(F)F)C[C@H]1c1ccc(C(=O)O)c2c1OCCN2]

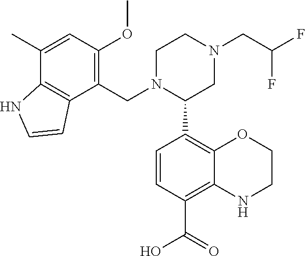 [SMILES: COc1cc(C)c2[nH]ccc2c1CN1CCN(CC(F)F)C[C@@H]1c1ccc(C(=O)O)c2c1OCCN2]

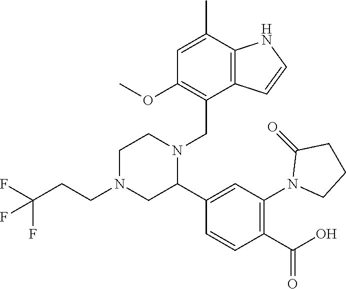 [SMILES: COc1cc(C)c2[nH]ccc2c1CN1CCN(CCC(F)(F)F)CC1c1ccc(C(=O)O)c(N2CCCC2=O)c1]

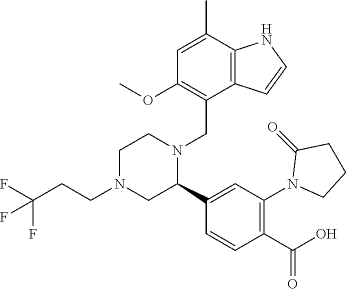 [SMILES: COc1cc(C)c2[nH]ccc2c1CN1CCN(CCC(F)(F)F)C[C@@H]1c1ccc(C(=O)O)c(N2CCCC2=O)c1]

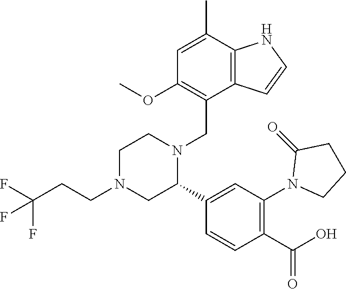 [SMILES: COc1cc(C)c2[nH]ccc2c1CN1CCN(CCC(F)(F)F)C[C@H]1c1ccc(C(=O)O)c(N2CCCC2=O)c1]

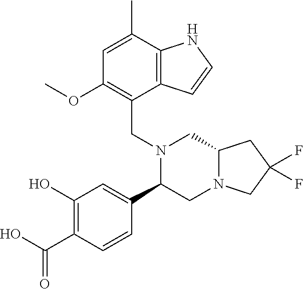 [SMILES: COc1cc(C)c2[nH]ccc2c1CN1C[C@@H]2CC(F)(F)CN2C[C@H]1c1ccc(C(=O)O)c(O)c1]

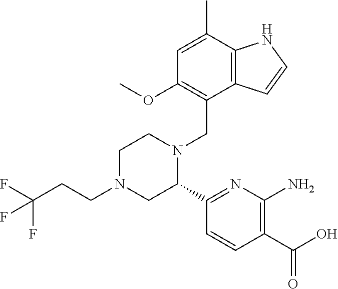 [SMILES: COc1cc(C)c2[nH]ccc2c1CN1CCN(CCC(F)(F)F)C[C@H]1c1ccc(C(=O)O)c(N)n1]

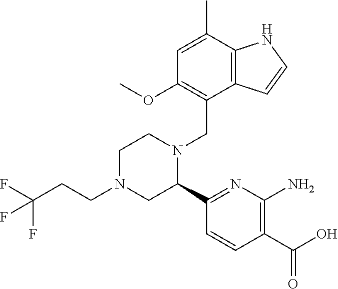 [SMILES: COc1cc(C)c2[nH]ccc2c1CN1CCN(CCC(F)(F)F)C[C@@H]1c1ccc(C(=O)O)c(N)n1]

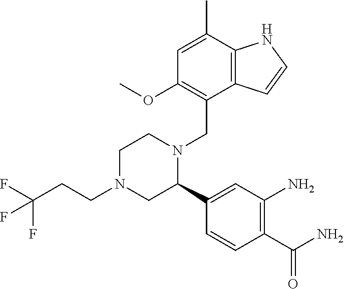 [SMILES: COc1cc(C)c2[nH]ccc2c1CN1CCN(CCC(F)(F)F)C[C@@H]1c1ccc(C(N)=O)c(N)c1]

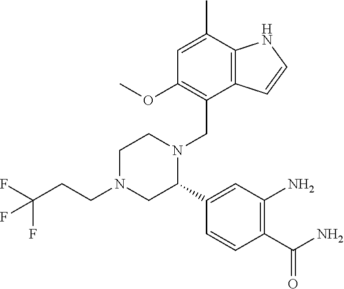 [SMILES: COc1cc(C)c2[nH]ccc2c1CN1CCN(CCC(F)(F)F)C[C@H]1c1ccc(C(N)=O)c(N)c1]

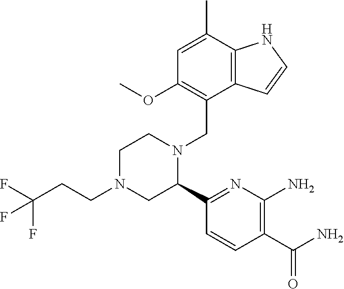 [SMILES: COc1cc(C)c2[nH]ccc2c1CN1CCN(CCC(F)(F)F)C[C@@H]1c1ccc(C(N)=O)c(N)n1]

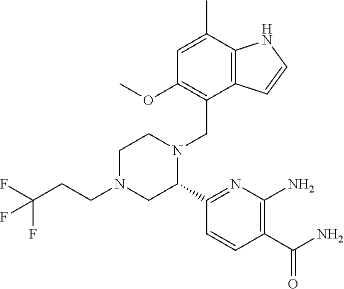 [SMILES: COc1cc(C)c2[nH]ccc2c1CN1CCN(CCC(F)(F)F)C[C@H]1c1ccc(C(N)=O)c(N)n1]

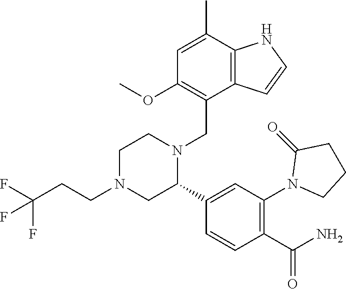 [SMILES: COc1cc(C)c2[nH]ccc2c1CN1CCN(CCC(F)(F)F)C[C@H]1c1ccc(C(N)=O)c(N2CCCC2=O)c1]

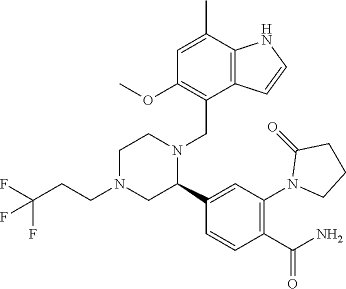 [SMILES: COc1cc(C)c2[nH]ccc2c1CN1CCN(CCC(F)(F)F)C[C@@H]1c1ccc(C(N)=O)c(N2CCCC2=O)c1]

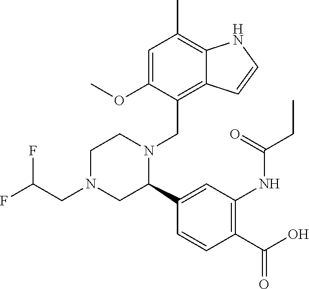 [SMILES: CCC(=O)Nc1cc([C@H]2CN(CC(F)F)CCN2Cc2c(OC)cc(C)c3[nH]ccc23)ccc1C(=O)O]

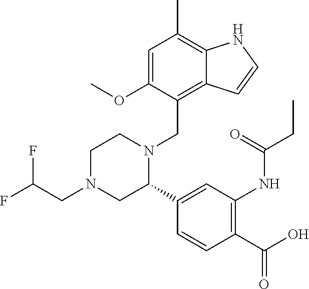 [SMILES: CCC(=O)Nc1cc([C@@H]2CN(CC(F)F)CCN2Cc2c(OC)cc(C)c3[nH]ccc23)ccc1C(=O)O]

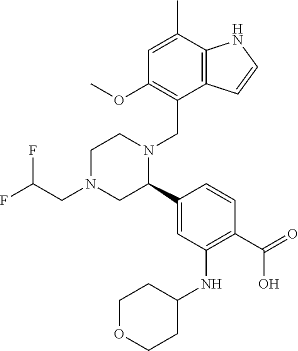 [SMILES: COc1cc(C)c2[nH]ccc2c1CN1CCN(CC(F)F)C[C@@H]1c1ccc(C(=O)O)c(NC2CCOCC2)c1]

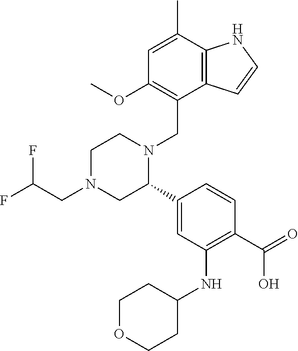 [SMILES: COc1cc(C)c2[nH]ccc2c1CN1CCN(CC(F)F)C[C@H]1c1ccc(C(=O)O)c(NC2CCOCC2)c1]

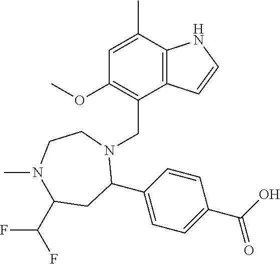 [SMILES: COc1cc(C)c2[nH]ccc2c1CN1CCN(C)C(C(F)F)CC1c1ccc(C(=O)O)cc1]

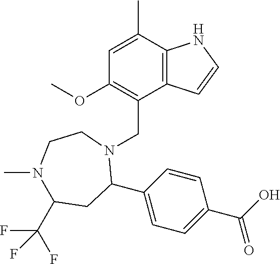 [SMILES: COc1cc(C)c2[nH]ccc2c1CN1CCN(C)C(C(F)(F)F)CC1c1ccc(C(=O)O)cc1]

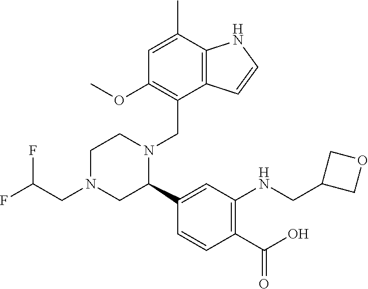 [SMILES: COc1cc(C)c2[nH]ccc2c1CN1CCN(CC(F)F)C[C@@H]1c1ccc(C(=O)O)c(NCC2COC2)c1]

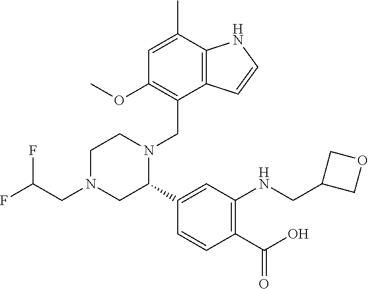 [SMILES: COc1cc(C)c2[nH]ccc2c1CN1CCN(CC(F)F)C[C@H]1c1ccc(C(=O)O)c(NCC2COC2)c1]

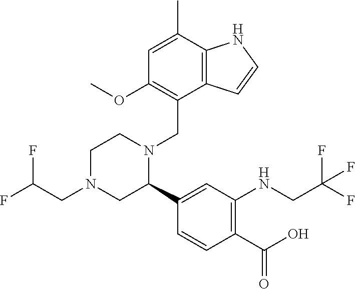 [SMILES: COc1cc(C)c2[nH]ccc2c1CN1CCN(CC(F)F)C[C@@H]1c1ccc(C(=O)O)c(NCC(F)(F)F)c1]

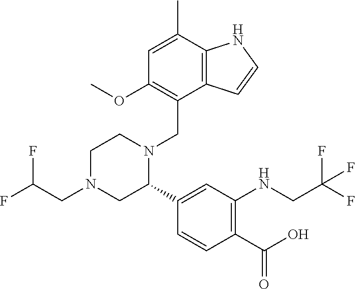 [SMILES: COc1cc(C)c2[nH]ccc2c1CN1CCN(CC(F)F)C[C@H]1c1ccc(C(=O)O)c(NCC(F)(F)F)c1]

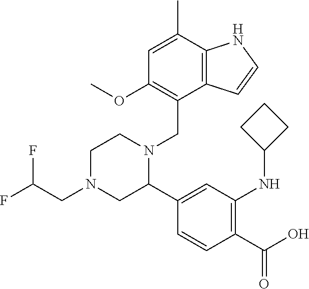 [SMILES: COc1cc(C)c2[nH]ccc2c1CN1CCN(CC(F)F)CC1c1ccc(C(=O)O)c(NC2CCC2)c1]

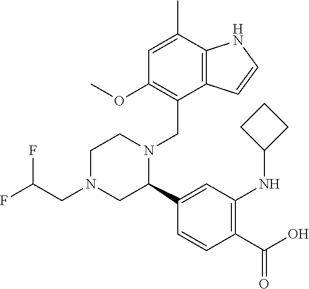 [SMILES: COc1cc(C)c2[nH]ccc2c1CN1CCN(CC(F)F)C[C@@H]1c1ccc(C(=O)O)c(NC2CCC2)c1]

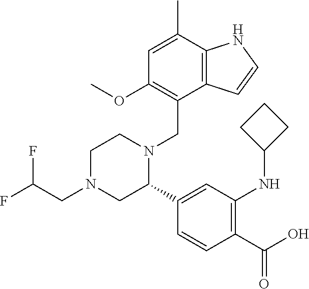 [SMILES: COc1cc(C)c2[nH]ccc2c1CN1CCN(CC(F)F)C[C@H]1c1ccc(C(=O)O)c(NC2CCC2)c1]

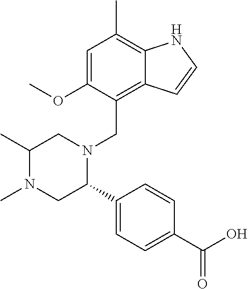 [SMILES: COc1cc(C)c2[nH]ccc2c1CN1CC(C)N(C)C[C@H]1c1ccc(C(=O)O)cc1]

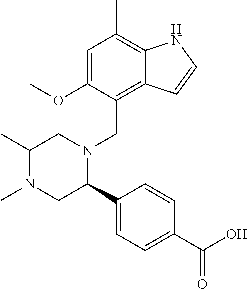 [SMILES: COc1cc(C)c2[nH]ccc2c1CN1CC(C)N(C)C[C@@H]1c1ccc(C(=O)O)cc1]

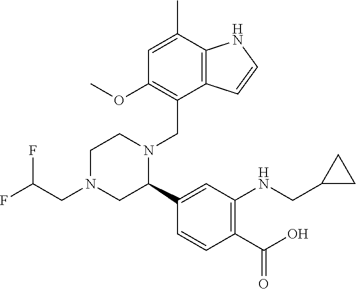 [SMILES: COc1cc(C)c2[nH]ccc2c1CN1CCN(CC(F)F)C[C@@H]1c1ccc(C(=O)O)c(NCC2CC2)c1]

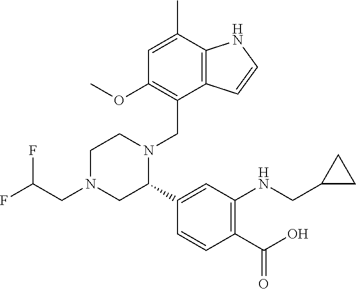 [SMILES: COc1cc(C)c2[nH]ccc2c1CN1CCN(CC(F)F)C[C@H]1c1ccc(C(=O)O)c(NCC2CC2)c1]

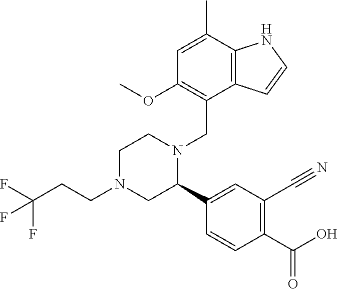 [SMILES: COc1cc(C)c2[nH]ccc2c1CN1CCN(CCC(F)(F)F)C[C@@H]1c1ccc(C(=O)O)c(C#N)c1]

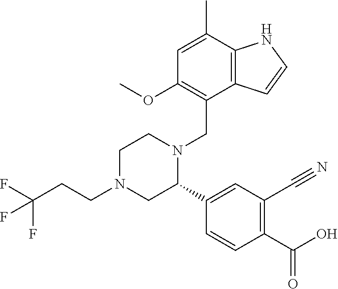 [SMILES: COc1cc(C)c2[nH]ccc2c1CN1CCN(CCC(F)(F)F)C[C@H]1c1ccc(C(=O)O)c(C#N)c1]

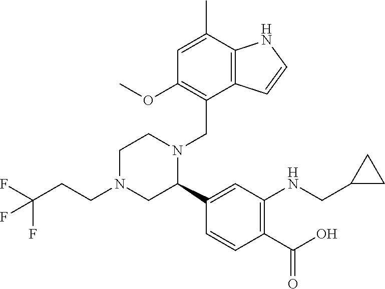 [SMILES: COc1cc(C)c2[nH]ccc2c1CN1CCN(CCC(F)(F)F)C[C@@H]1c1ccc(C(=O)O)c(NCC2CC2)c1]

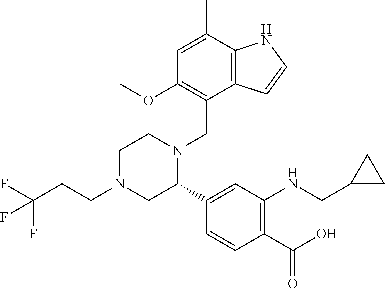 [SMILES: COc1cc(C)c2[nH]ccc2c1CN1CCN(CCC(F)(F)F)C[C@H]1c1ccc(C(=O)O)c(NCC2CC2)c1]

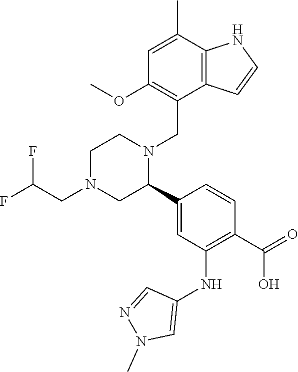 [SMILES: COc1cc(C)c2[nH]ccc2c1CN1CCN(CC(F)F)C[C@@H]1c1ccc(C(=O)O)c(Nc2cnn(C)c2)c1]

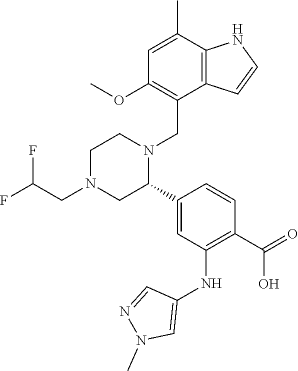 [SMILES: COc1cc(C)c2[nH]ccc2c1CN1CCN(CC(F)F)C[C@H]1c1ccc(C(=O)O)c(Nc2cnn(C)c2)c1]

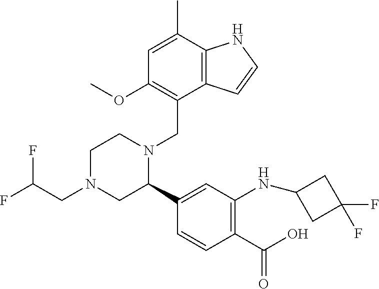 [SMILES: COc1cc(C)c2[nH]ccc2c1CN1CCN(CC(F)F)C[C@@H]1c1ccc(C(=O)O)c(NC2CC(F)(F)C2)c1]

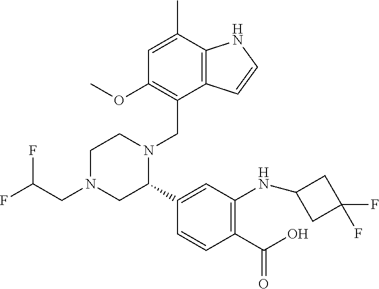 [SMILES: COc1cc(C)c2[nH]ccc2c1CN1CCN(CC(F)F)C[C@H]1c1ccc(C(=O)O)c(NC2CC(F)(F)C2)c1]

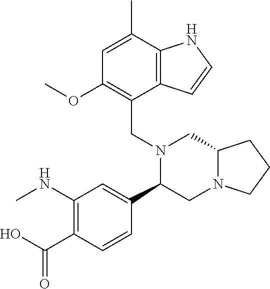 [SMILES: CNc1cc([C@@H]2CN3CCC[C@H]3CN2Cc2c(OC)cc(C)c3[nH]ccc23)ccc1C(=O)O]

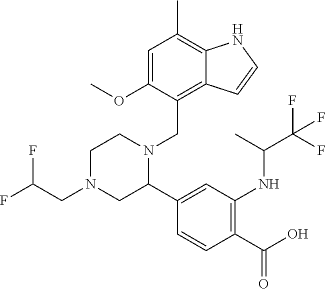 [SMILES: COc1cc(C)c2[nH]ccc2c1CN1CCN(CC(F)F)CC1c1ccc(C(=O)O)c(NC(C)C(F)(F)F)c1]

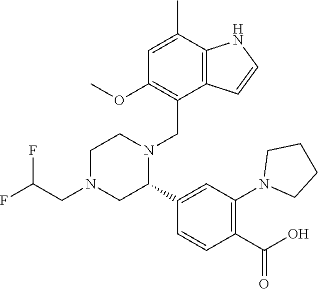 [SMILES: COc1cc(C)c2[nH]ccc2c1CN1CCN(CC(F)F)C[C@H]1c1ccc(C(=O)O)c(N2CCCC2)c1]

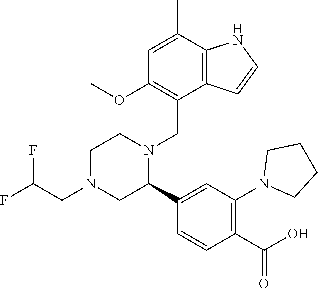 [SMILES: COc1cc(C)c2[nH]ccc2c1CN1CCN(CC(F)F)C[C@@H]1c1ccc(C(=O)O)c(N2CCCC2)c1]

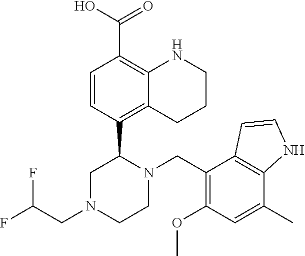 [SMILES: COc1cc(C)c2[nH]ccc2c1CN1CCN(CC(F)F)C[C@H]1c1ccc(C(=O)O)c2c1CCCN2]

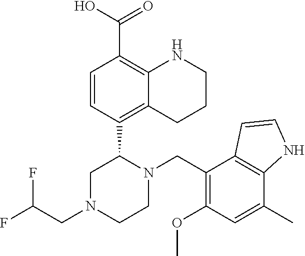 [SMILES: COc1cc(C)c2[nH]ccc2c1CN1CCN(CC(F)F)C[C@@H]1c1ccc(C(=O)O)c2c1CCCN2]

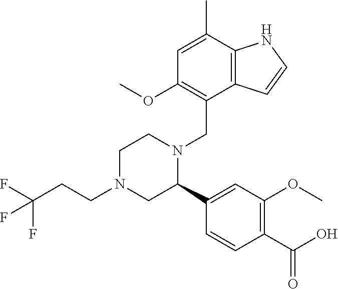 [SMILES: COc1cc([C@H]2CN(CCC(F)(F)F)CCN2Cc2c(OC)cc(C)c3[nH]ccc23)ccc1C(=O)O]

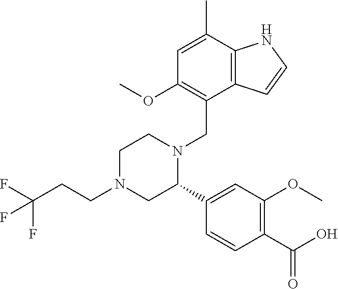 [SMILES: COc1cc([C@@H]2CN(CCC(F)(F)F)CCN2Cc2c(OC)cc(C)c3[nH]ccc23)ccc1C(=O)O]

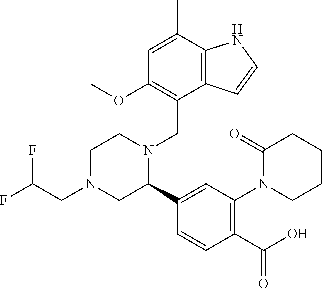 [SMILES: COc1cc(C)c2[nH]ccc2c1CN1CCN(CC(F)F)C[C@@H]1c1ccc(C(=O)O)c(N2CCCCC2=O)c1]

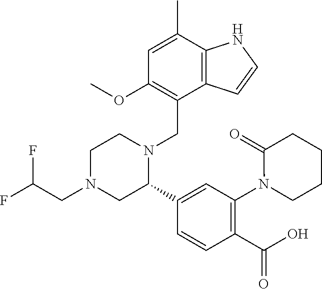 [SMILES: COc1cc(C)c2[nH]ccc2c1CN1CCN(CC(F)F)C[C@H]1c1ccc(C(=O)O)c(N2CCCCC2=O)c1]

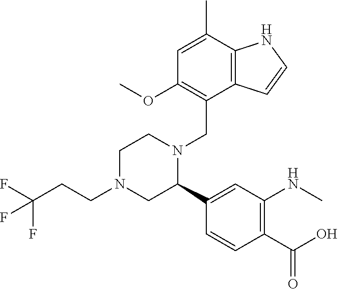 [SMILES: CNc1cc([C@H]2CN(CCC(F)(F)F)CCN2Cc2c(OC)cc(C)c3[nH]ccc23)ccc1C(=O)O]

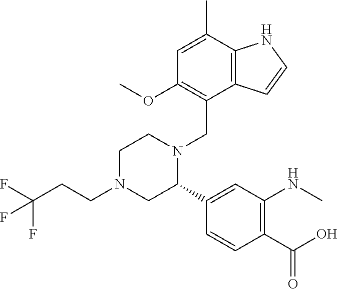 [SMILES: CNc1cc([C@@H]2CN(CCC(F)(F)F)CCN2Cc2c(OC)cc(C)c3[nH]ccc23)ccc1C(=O)O]

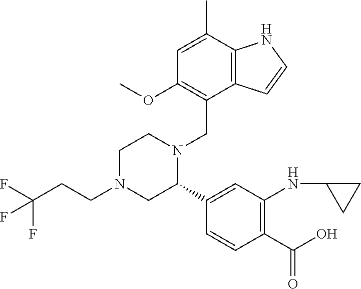 [SMILES: COc1cc(C)c2[nH]ccc2c1CN1CCN(CCC(F)(F)F)C[C@H]1c1ccc(C(=O)O)c(NC2CC2)c1]

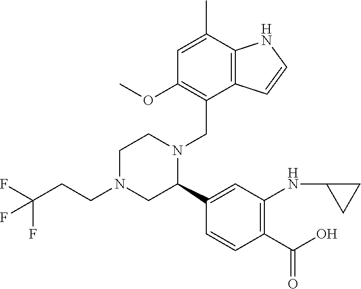 [SMILES: COc1cc(C)c2[nH]ccc2c1CN1CCN(CCC(F)(F)F)C[C@@H]1c1ccc(C(=O)O)c(NC2CC2)c1]

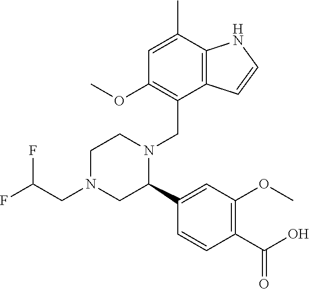 [SMILES: COc1cc([C@H]2CN(CC(F)F)CCN2Cc2c(OC)cc(C)c3[nH]ccc23)ccc1C(=O)O]

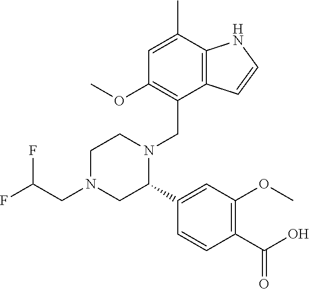 [SMILES: COc1cc([C@@H]2CN(CC(F)F)CCN2Cc2c(OC)cc(C)c3[nH]ccc23)ccc1C(=O)O]

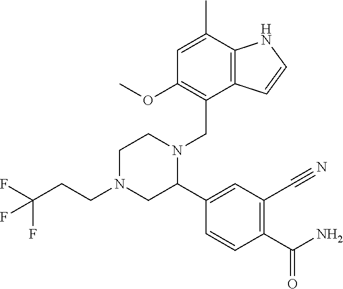 [SMILES: COc1cc(C)c2[nH]ccc2c1CN1CCN(CCC(F)(F)F)CC1c1ccc(C(N)=O)c(C#N)c1]

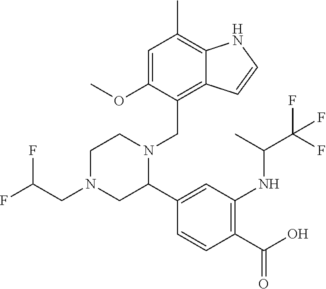 [SMILES: COc1cc(C)c2[nH]ccc2c1CN1CCN(CC(F)F)CC1c1ccc(C(=O)O)c(NC(C)C(F)(F)F)c1]

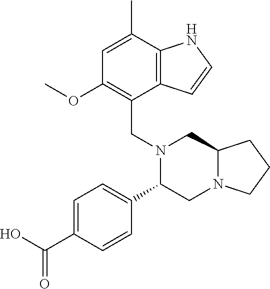 [SMILES: COc1cc(C)c2[nH]ccc2c1CN1C[C@H]2CCCN2C[C@@H]1c1ccc(C(=O)O)cc1]

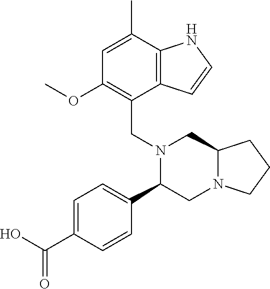 [SMILES: COc1cc(C)c2[nH]ccc2c1CN1C[C@H]2CCCN2C[C@H]1c1ccc(C(=O)O)cc1]

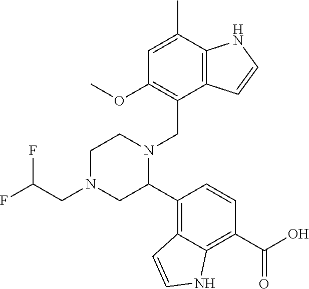 [SMILES: COc1cc(C)c2[nH]ccc2c1CN1CCN(CC(F)F)CC1c1ccc(C(=O)O)c2[nH]ccc12]

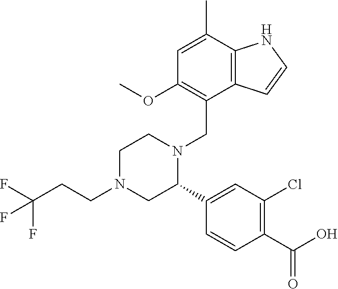 [SMILES: COc1cc(C)c2[nH]ccc2c1CN1CCN(CCC(F)(F)F)C[C@H]1c1ccc(C(=O)O)c(Cl)c1]

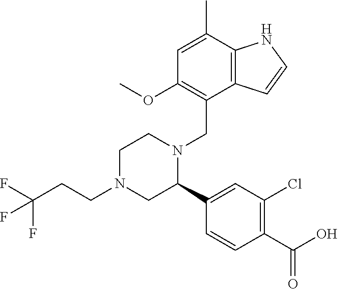 [SMILES: COc1cc(C)c2[nH]ccc2c1CN1CCN(CCC(F)(F)F)C[C@@H]1c1ccc(C(=O)O)c(Cl)c1]

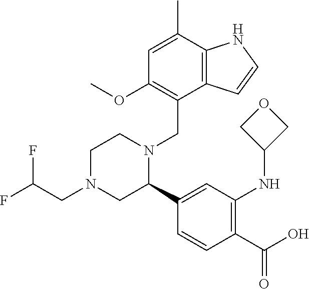 [SMILES: COc1cc(C)c2[nH]ccc2c1CN1CCN(CC(F)F)C[C@@H]1c1ccc(C(=O)O)c(NC2COC2)c1]

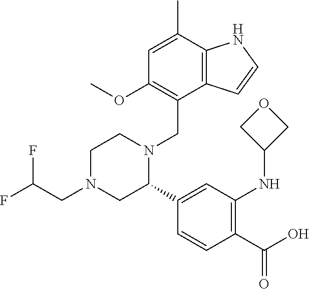 [SMILES: COc1cc(C)c2[nH]ccc2c1CN1CCN(CC(F)F)C[C@H]1c1ccc(C(=O)O)c(NC2COC2)c1]

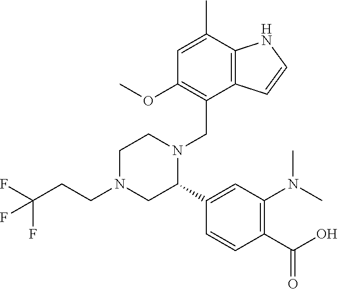 [SMILES: COc1cc(C)c2[nH]ccc2c1CN1CCN(CCC(F)(F)F)C[C@H]1c1ccc(C(=O)O)c(N(C)C)c1]

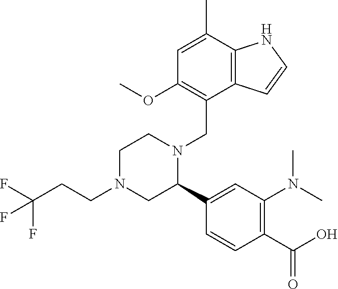 [SMILES: COc1cc(C)c2[nH]ccc2c1CN1CCN(CCC(F)(F)F)C[C@@H]1c1ccc(C(=O)O)c(N(C)C)c1]

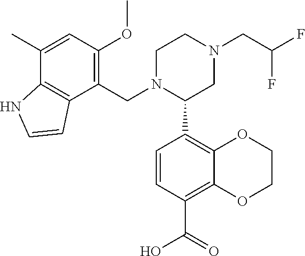 [SMILES: COc1cc(C)c2[nH]ccc2c1CN1CCN(CC(F)F)C[C@@H]1c1ccc(C(=O)O)c2c1OCCO2]

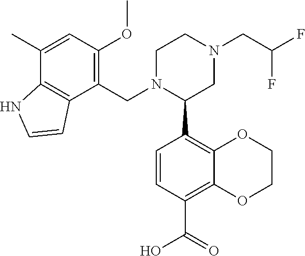 [SMILES: COc1cc(C)c2[nH]ccc2c1CN1CCN(CC(F)F)C[C@H]1c1ccc(C(=O)O)c2c1OCCO2]

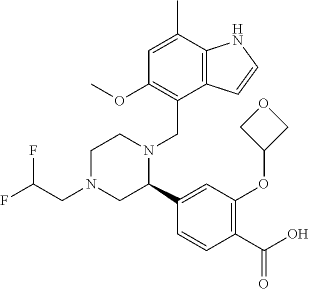 [SMILES: COc1cc(C)c2[nH]ccc2c1CN1CCN(CC(F)F)C[C@@H]1c1ccc(C(=O)O)c(OC2COC2)c1]

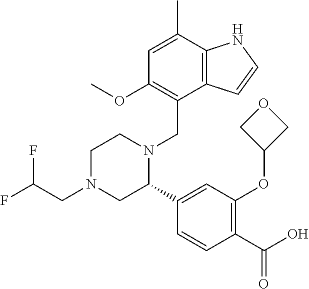 [SMILES: COc1cc(C)c2[nH]ccc2c1CN1CCN(CC(F)F)C[C@H]1c1ccc(C(=O)O)c(OC2COC2)c1]

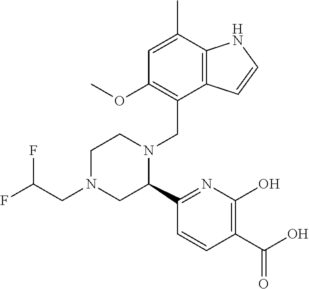 [SMILES: COc1cc(C)c2[nH]ccc2c1CN1CCN(CC(F)F)C[C@@H]1c1ccc(C(=O)O)c(O)n1]

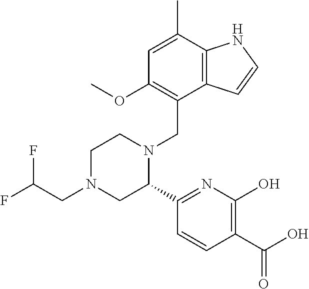 [SMILES: COc1cc(C)c2[nH]ccc2c1CN1CCN(CC(F)F)C[C@H]1c1ccc(C(=O)O)c(O)n1]

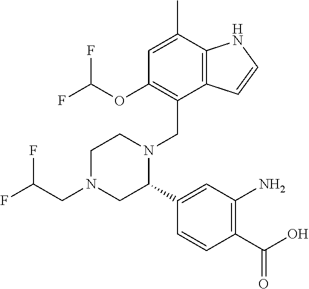 [SMILES: Cc1cc(OC(F)F)c(CN2CCN(CC(F)F)C[C@H]2c2ccc(C(=O)O)c(N)c2)c2cc[nH]c12]

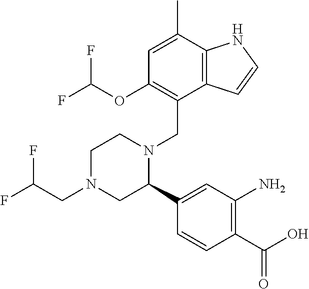 [SMILES: Cc1cc(OC(F)F)c(CN2CCN(CC(F)F)C[C@@H]2c2ccc(C(=O)O)c(N)c2)c2cc[nH]c12]

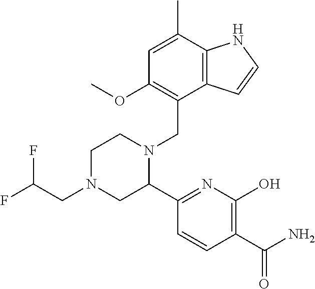 [SMILES: COc1cc(C)c2[nH]ccc2c1CN1CCN(CC(F)F)CC1c1ccc(C(N)=O)c(O)n1]

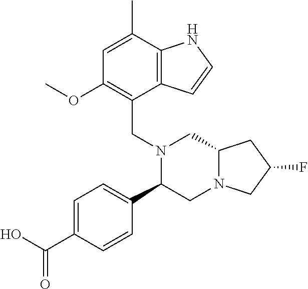 [SMILES: COc1cc(C)c2[nH]ccc2c1CN1C[C@@H]2C[C@H](F)CN2C[C@H]1c1ccc(C(=O)O)cc1]

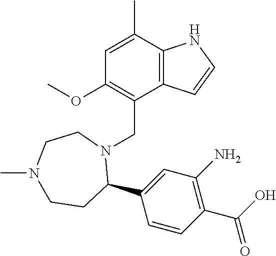 [SMILES: COc1cc(C)c2[nH]ccc2c1CN1CCN(C)CC[C@@H]1c1ccc(C(=O)O)c(N)c1]

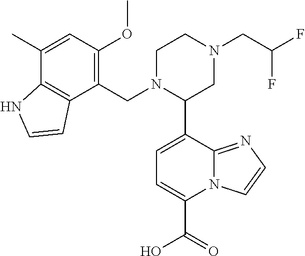 [SMILES: COc1cc(C)c2[nH]ccc2c1CN1CCN(CC(F)F)CC1c1ccc(C(=O)O)n2ccnc12]

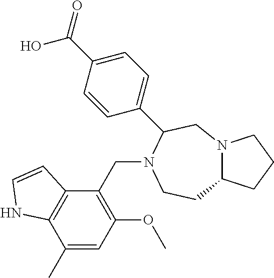

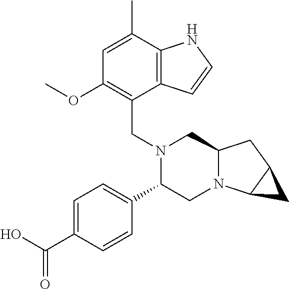 [SMILES: COc1cc(C)c2[nH]ccc2c1CN1C[C@H]2C[C@H]3C[C@H]3N2C[C@@H]1c1ccc(C(=O)O)cc1]

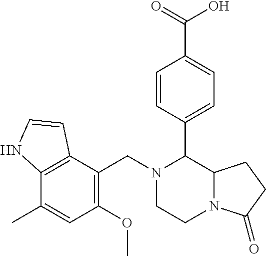 [SMILES: COc1cc(C)c2[nH]ccc2c1CN1CCN2C(=O)CCC2C1c1ccc(C(=O)O)cc1]

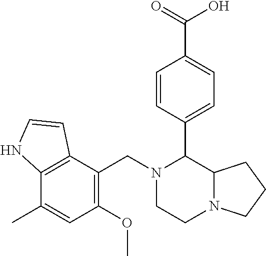 [SMILES: COc1cc(C)c2[nH]ccc2c1CN1CCN2CCCC2C1c1ccc(C(=O)O)cc1]

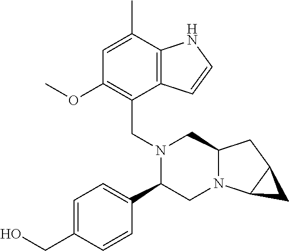 [SMILES: COc1cc(C)c2[nH]ccc2c1CN1C[C@H]2C[C@H]3C[C@H]3N2C[C@H]1c1ccc(CO)cc1]

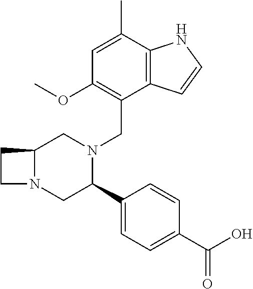 [SMILES: COc1cc(C)c2[nH]ccc2c1CN1C[C@@H]2CCN2C[C@@H]1c1ccc(C(=O)O)cc1]

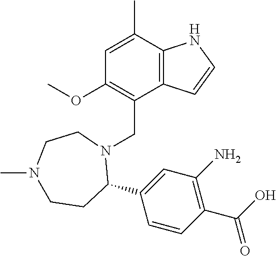 [SMILES: COc1cc(C)c2[nH]ccc2c1CN1CCN(C)CC[C@H]1c1ccc(C(=O)O)c(N)c1]

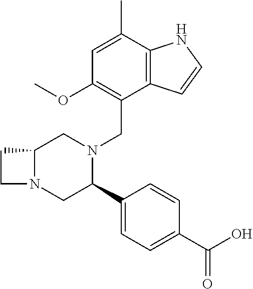 [SMILES: COc1cc(C)c2[nH]ccc2c1CN1C[C@H]2CCN2C[C@@H]1c1ccc(C(=O)O)cc1]

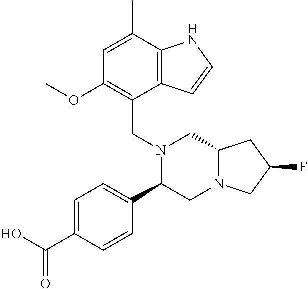 [SMILES: COc1cc(C)c2[nH]ccc2c1CN1C[C@@H]2C[C@@H](F)CN2C[C@H]1c1ccc(C(=O)O)cc1]

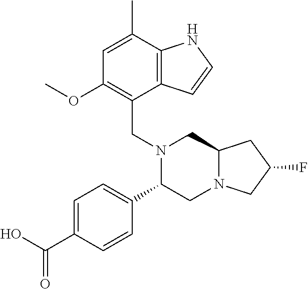 [SMILES: COc1cc(C)c2[nH]ccc2c1CN1C[C@H]2C[C@H](F)CN2C[C@@H]1c1ccc(C(=O)O)cc1]

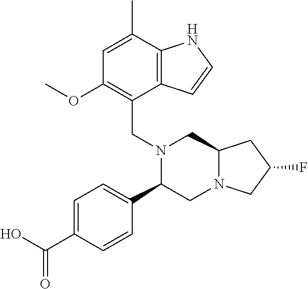 [SMILES: COc1cc(C)c2[nH]ccc2c1CN1C[C@H]2C[C@H](F)CN2C[C@H]1c1ccc(C(=O)O)cc1]

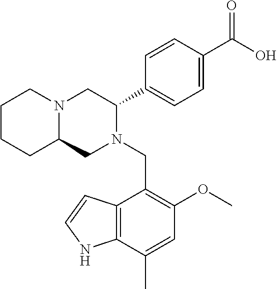 [SMILES: COc1cc(C)c2[nH]ccc2c1CN1C[C@H]2CCCCN2C[C@@H]1c1ccc(C(=O)O)cc1]

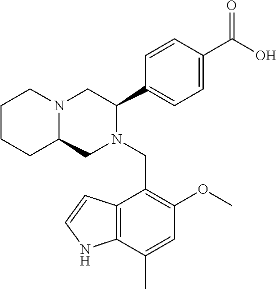 [SMILES: COc1cc(C)c2[nH]ccc2c1CN1C[C@H]2CCCCN2C[C@H]1c1ccc(C(=O)O)cc1]

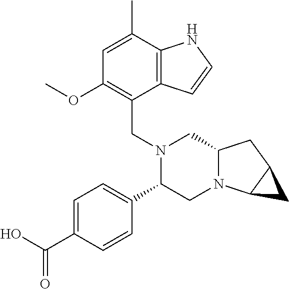 [SMILES: COc1cc(C)c2[nH]ccc2c1CN1C[C@@H]2C[C@H]3C[C@H]3N2C[C@@H]1c1ccc(C(=O)O)cc1]

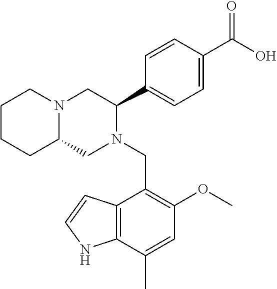 [SMILES: COc1cc(C)c2[nH]ccc2c1CN1C[C@@H]2CCCCN2C[C@H]1c1ccc(C(=O)O)cc1]

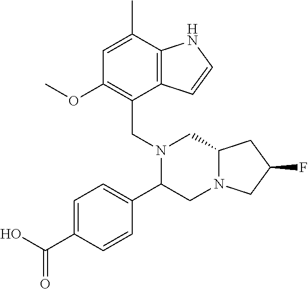

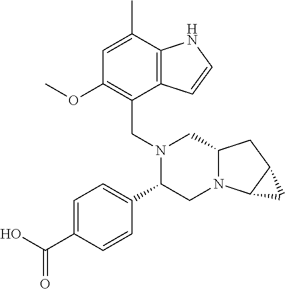 [SMILES: COc1cc(C)c2[nH]ccc2c1CN1C[C@@H]2C[C@@H]3C[C@@H]3N2C[C@@H]1c1ccc(C(=O)O)cc1]

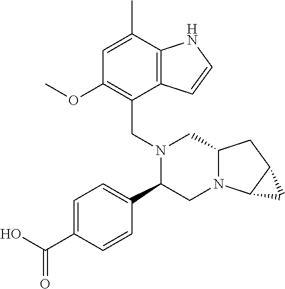 [SMILES: COc1cc(C)c2[nH]ccc2c1CN1C[C@@H]2C[C@@H]3C[C@@H]3N2C[C@H]1c1ccc(C(=O)O)cc1]

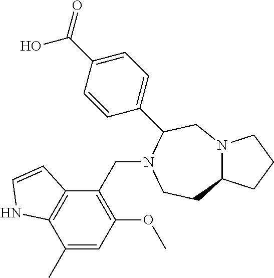

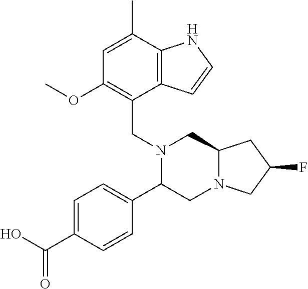

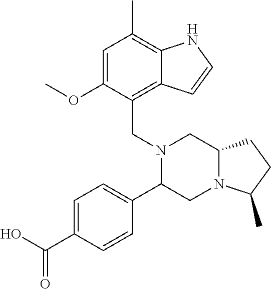 [SMILES: COc1cc(C)c2[nH]ccc2c1CN1C[C@@H]2CC[C@@H](C)N2CC1c1ccc(C(=O)O)cc1]

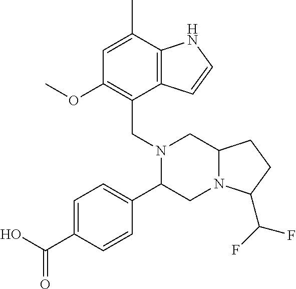 [SMILES: COc1cc(C)c2[nH]ccc2c1CN1CC2CCC(C(F)F)N2CC1c1ccc(C(=O)O)cc1]

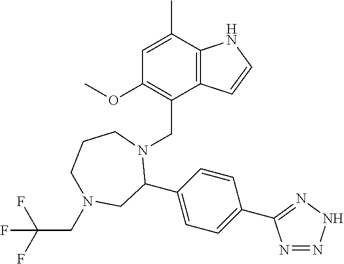 [SMILES: COc1cc(C)c2[nH]ccc2c1CN1CCCN(CC(F)(F)F)CC1c1ccc(-c2nn[nH]n2)cc1]

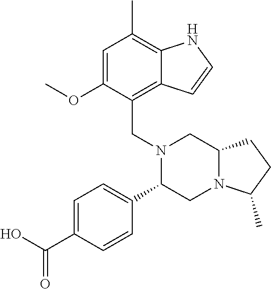 [SMILES: COc1cc(C)c2[nH]ccc2c1CN1C[C@@H]2CC[C@H](C)N2C[C@@H]1c1ccc(C(=O)O)cc1]

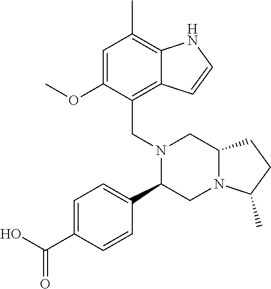 [SMILES: COc1cc(C)c2[nH]ccc2c1CN1C[C@@H]2CC[C@H](C)N2C[C@H]1c1ccc(C(=O)O)cc1]

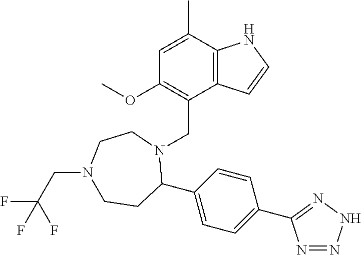 [SMILES: COc1cc(C)c2[nH]ccc2c1CN1CCN(CC(F)(F)F)CCC1c1ccc(-c2nn[nH]n2)cc1]

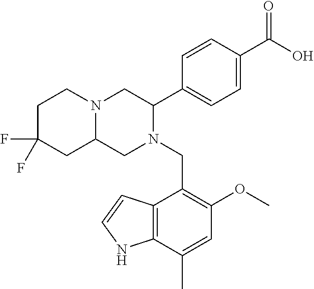 [SMILES: COc1cc(C)c2[nH]ccc2c1CN1CC2CC(F)(F)CCN2CC1c1ccc(C(=O)O)cc1]

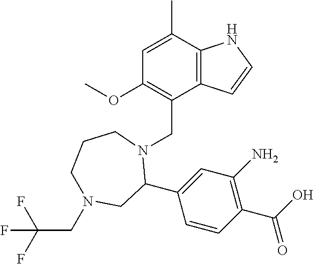 [SMILES: COc1cc(C)c2[nH]ccc2c1CN1CCCN(CC(F)(F)F)CC1c1ccc(C(=O)O)c(N)c1]

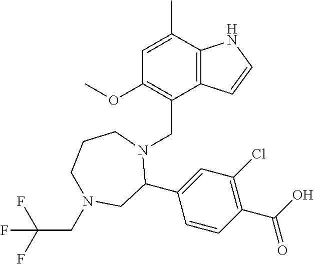 [SMILES: COc1cc(C)c2[nH]ccc2c1CN1CCCN(CC(F)(F)F)CC1c1ccc(C(=O)O)c(Cl)c1]

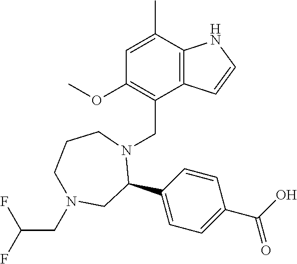 [SMILES: COc1cc(C)c2[nH]ccc2c1CN1CCCN(CC(F)F)C[C@@H]1c1ccc(C(=O)O)cc1]

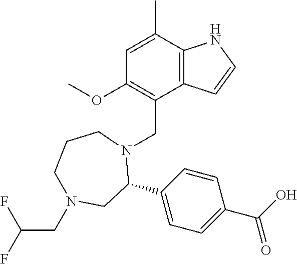 [SMILES: COc1cc(C)c2[nH]ccc2c1CN1CCCN(CC(F)F)C[C@H]1c1ccc(C(=O)O)cc1]

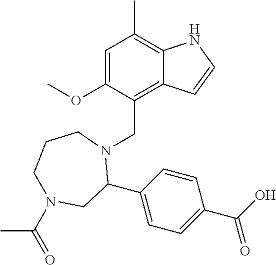 [SMILES: COc1cc(C)c2[nH]ccc2c1CN1CCCN(C(C)=O)CC1c1ccc(C(=O)O)cc1]

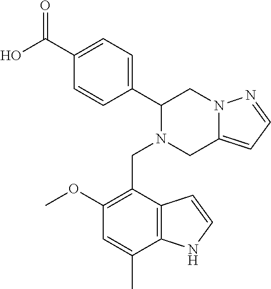 [SMILES: COc1cc(C)c2[nH]ccc2c1CN1Cc2ccnn2CC1c1ccc(C(=O)O)cc1]

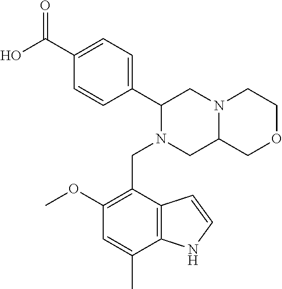 [SMILES: COc1cc(C)c2[nH]ccc2c1CN1CC2COCCN2CC1c1ccc(C(=O)O)cc1]

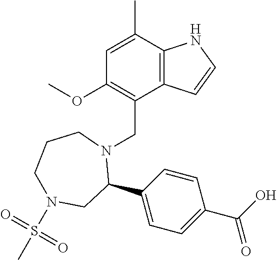 [SMILES: COc1cc(C)c2[nH]ccc2c1CN1CCCN(S(C)(=O)=O)C[C@@H]1c1ccc(C(=O)O)cc1]

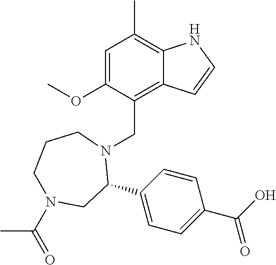 [SMILES: COc1cc(C)c2[nH]ccc2c1CN1CCCN(C(C)=O)C[C@H]1c1ccc(C(=O)O)cc1]

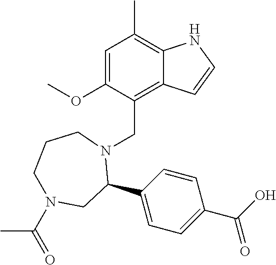 [SMILES: COc1cc(C)c2[nH]ccc2c1CN1CCCN(C(C)=O)C[C@@H]1c1ccc(C(=O)O)cc1]

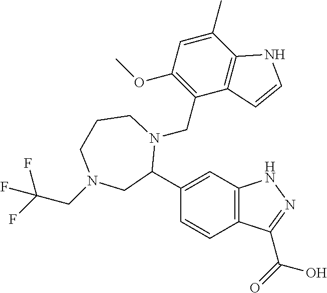 [SMILES: COc1cc(C)c2[nH]ccc2c1CN1CCCN(CC(F)(F)F)CC1c1ccc2c(C(=O)O)n[nH]c2c1]

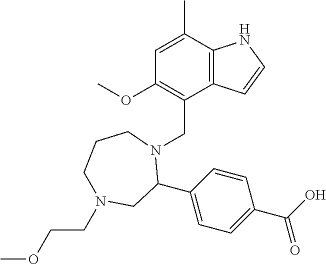 [SMILES: COCCN1CCCN(Cc2c(OC)cc(C)c3[nH]ccc23)C(c2ccc(C(=O)O)cc2)C1]

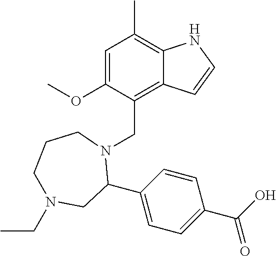 [SMILES: CCN1CCCN(Cc2c(OC)cc(C)c3[nH]ccc23)C(c2ccc(C(=O)O)cc2)C1]

In some embodiments, compounds of the present disclosure comprise one or more chiral elements, e.g., chiral centers. In some embodiments, compounds are utilized as a mixture of two or more stereoisomers. In some embodiments, compounds are utilized as a mixture of enantiomers. In some embodiments, certain stereoisomer(s) may provide better activities, properties (e.g., lower toxicities), etc. compared to others. In some embodiments, an enantiomer may provide better activities, properties (e.g., lower toxicities), etc. than the other. In some embodiments, single stereoisomers are utilized. In some embodiments, a single enantiomer is utilized.

In some embodiments, a compound has a purity of about 95%-100% (e.g., about 95%, 96%, 97%, 98%, 99%, 99.5%, 99.9% or more, or about 96%-100%, 97%-100%, 98%-100%, 99%-100%, 95-99%, 95%-99.5%, 95%-99.9%, etc). In some embodiments, a compound has a diastereomeric purity of about 95%-100% (e.g., about 95%, 96%, 97%, 98%, 99%, 99.5%, 99.9% or more, or about 96%-100%, 97%-100%, 98%-100%, 99%-100%, 95-99%, 95%-99.5%, 95%-99.9%, etc). In some embodiments, a compound has an enantiomeric purity of about 95%-100% (e.g., about 95%, 96%, 97%, 98%, 99%, 99.5%, 99.9% or more, or about 96%-100%, 97%-100%, 98%-100%, 99%-100%, 95-99%, 95%-99.5%, 95%-99.9%, etc). In some embodiments, a percentage is weight percentage. In some embodiments, a percentage is about or at least about 95%. In some embodiments, a percentage is about or at least about 96%. In some embodiments, a percentage is about or at least about 97%. In some embodiments, a percentage is about or at least about 98%. In some embodiments, a percentage is about or at least about 99%. In some embodiments, a percentage is about or at least about 99.5%. In some embodiments, a percentage is about or at least about 99.9%. In some embodiments, a percentage is about 100%.

### III. Compositions and Administration

In some embodiments, compounds are administered as pharmaceutical compositions. In some embodiments, the present disclosure provides a pharmaceutical composition comprising a provided compound, e.g., a compound of formula I or a pharmaceutically acceptable salt thereof, and a pharmaceutically acceptable carrier. In some embodiments, a composition, e.g. a pharmaceutical composition, delivers a provided compound, e.g., a compound of formula I or a pharmaceutically acceptable salt thereof. In some embodiments, agents, e.g., compounds of formula I or pharmaceutically acceptable salts thereof and compositions are useful for treating various conditions, disorders or diseases, e.g., complement-mediated conditions, disorders or diseases, C3 convertase-mediated conditions, disorders or diseases, etc.

Various technologies, e.g., routes, modes, dosage regimens, etc. may be utilized to administer and/or deliver provided compounds and compositions in accordance with the present disclosure. In some embodiments, a route and/or mode of administration can vary depending upon desired result(s). One with skill in the art, i.e., a physician, is aware that dosage regimens can be adjusted to provide a desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a mode of administration is left to the discretion of a practitioner.

In some embodiments, compounds can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such carriers or excipients include any pharmaceutical agent, e.g., a pharmaceutical agent that does not itself induce an immune response harmful to the individual receiving a composition, and which may be administered without undue toxicity. Pharmaceutically acceptable carriers or excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Compounds in pharmaceutical compositions may be provided as pharmaceutically acceptable salts. In some embodiments, salts can be formed with many acids, including but not limited to, hydrochloric, sulfuric, acetic, lactic, tartaric, malic, succinic, benzenesulfonic, etc. In some embodiments, salts can be formed with bases. In some embodiments, salts are alkali, alkaline earth metal, or ammonium salts, e.g., sodium, calcium, trialkylamine salts, etc.

In some embodiments, salts are more soluble in aqueous or other protonic solvents than corresponding, free acid or base forms. In some embodiments, a pharmaceutical composition may be a lyophilized powder. In some embodiments, a pharmaceutical composition comprises a provided compound, e.g., a compound of formula I or a pharmaceutically acceptable salt thereof dissolved in a pharmaceutically acceptable buffer. In some embodiments, a buffer is a saline buffer. In some embodiments, a buffer has a pH around 7.4.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. In some embodiments, pharmaceutical compositions comprise carriers, diluents, or excipients suitable for administration by various routes.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Cosolvents and adjuvants may be added to compositions and formulations. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Various pharmaceutical compositions and delivery systems appropriate for compositions, methods and uses of the present disclosure are known in the art (see, e.g., Remington: The Science and Practice of Pharmacy. 21st Edition. Philadelphia, PA. Lippincott Williams & Wilkins, 2005) and can be utilized in accordance with the present disclosure.

In some embodiments, the present disclosure provides methods for introducing provided compounds and compositions into cells, animals or subjects. In some embodiments, such methods include contacting a subject (e.g., a cell or tissue of a subject) with, or administering to a subject (e.g., a subject such as a mammal or human) a provided compound, e.g., a compound of formula I or a salt thereof, or a composition thereof.

A compound or composition described herein can be administered in a sufficient or effective amount to a subject (or a cell, tissue or organ thereof) in need thereof. Doses can vary and may depend upon the type, onset, progression, severity, frequency, duration, or probability of a condition, disorder or disease to which treatment is directed, a clinical endpoint desired, previous or simultaneous treatments, general health, age, gender, race or immunological competency of a subject and other factors that will be appreciated by a skilled artisan. Dose amount, number, frequency or duration may be proportionally increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit.

A dose to achieve a therapeutic effect will vary based on several factors including, but not limited to: route of administration, amount to achieve a therapeutic effect, the specific condition, disorder or disease treated, any host immune response to administered compound or composition, or stability of administered compound or composition.

An effective amount or a sufficient amount can (but need not) be provided in a single administration, may require multiple administrations, and, can (but need not) be, administered alone or in combination with another composition (e.g., comprising or delivering another therapeutic agent). For example, an amount may be proportionally increased as indicated by the need of a subject, type, status and severity of a condition, disorder or disease treated and/or side effects (if any) of treatment. Amounts considered effective also include amounts that result in a reduction of the use of another treatment, therapeutic regimen or protocol, such as administration of another complement inhibitor described herein.

In some embodiments, pharmaceutical compositions comprise or deliver active ingredients, e.g., compounds of formula I or pharmaceutically acceptable salts thereof, in effective amounts to achieve intended purposes e.g., therapeutic purposes. Various technologies may be utilized to determine therapeutically effective amounts in accordance with the present disclosure. Therapeutic doses can depend on, among other factors, ages and general conditions of subjects, severity of conditions, disorders or diseases (e.g., complement-mediated conditions, disorders or diseases, C3 convertase-mediated conditions, disorders or diseases, etc.), etc. Thus, therapeutically effective amounts in humans may fall in a relatively broad range that may be determined by medical practitioners based on responses of individual patients.

In some embodiments, methods and uses of the present disclosure include delivery and administration systemically, regionally or locally, or by any route, for example, by injection or infusion. In some embodiments, delivery of a pharmaceutical composition in vivo may generally be accomplished via injection using a conventional syringe, although other delivery methods such as convection-enhanced delivery can also be used (see, e.g., U.S. Pat. No. 5,720,720). In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. Clinicians specializing in treating patients with complement-mediated conditions, disorders or diseases may determine optimal routes for administration of compounds and compositions as described herein.

In some embodiments, a compound or composition may be administered to a subject four times a day, three times a day, twice a day, once daily, every 2, 3, 4, 5, or 6 days, weekly, or every 2, 3, or 4 weeks, or even at longer intervals. In some embodiments, a compound or composition is administered to a subject once, e.g., as a single injection or as a single infusion over time (e.g., over 5, 10, 15, 20, 30, 40, 50, 60, 90, 120 minutes, or longer). In some embodiments, a compound or composition is administered to a subject twice, e.g., as two injections (e.g., 2, 4, 6, 8, 10, or 12 hours apart) or as two infusions (e.g., 2, 4, 6, 8, 10, or 12 hours apart). In some embodiments, a subject is monitored before and/or following an administration or treatment for level of C3 expression and/or activity, a complement activity, etc., e.g., as measured using an alternative pathway assay, a classical pathway assay, or both. Suitable assays are known in the art and include, e.g., a hemolysis assay and those described in the Examples.

### IV. Diseases, Disorders, and Conditions

Provided technologies are useful for preventing or treating various conditions, disorders or diseases. In some embodiments, the present disclosure provides methods for preventing a condition, disorder or disease, comprising administering to a subject susceptible thereto an effective amount of a compound, e.g., a compound of formula I or a pharmaceutically acceptable salt. In some embodiments, the present disclosure provides methods for treating a condition, disorder or disease, comprising administering to a subject suffering therefrom a therapeutically effective amount of a compound, e.g., a compound of formula I or a pharmaceutically acceptable salt. In some embodiments, the present disclosure provides a method for reducing C3 convertase activity, comprising contacting a C3 convertase with a provided compound, e.g., a compound of formula I or a pharmaceutically acceptable salt thereof, or a composition thereof. In some embodiments, the present disclosure provides a method for reducing factor B activity, comprising contacting a factor B with a provided compound, e.g., a compound of formula I or a pharmaceutically acceptable salt thereof, or a composition thereof. In some embodiments, the present disclosure provides a method for reducing complement activation in a system, comprising administering to the system a provided compound, e.g., a compound of formula I or a pharmaceutically acceptable salt thereof, or a composition thereof. In some embodiments, the present disclosure provides a method for reducing C3 convertase activity in a system, comprising administering to the system a provided compound, e.g., a compound of formula I or a pharmaceutically acceptable salt thereof, or a composition thereof. In some embodiments, the present disclosure provides a method for reducing factor B activity in a system, comprising administering to the system a provided compound, e.g., a compound of formula I or a pharmaceutically acceptable salt thereof, or a composition thereof. In some embodiments, a system is a plurality of cells, a tissue, organ or organism. In some embodiments, a system is or comprises blood. In some embodiments, a system is an animal. In some embodiments, a system is a human. In some embodiments, a subject is a human.

In some embodiments, a condition, disorder or disease is a complement-mediated condition, disorder or disease. In some embodiments, a condition, disorder or disease is a C3 convertase-mediated condition, disorder or disease. In some embodiments, a condition, disorder or disease is a factor B-mediated condition, disorder or disease. In some embodiments, a condition, disorder or disease is or comprises complement-mediated damage to an organ, tissue, or cells. In some embodiments, a compound or composition is administered in combination with another therapeutic agent, e.g., a complement inhibitor.

### Blood-Related Disorders

In some embodiments, a compound is administered to a subject suffering from, or at risk of, a complement-mediated blood-related disorder, such as paroxysmal nocturnal hemoglobinuria (PNH), atypical hemolytic uremic syndrome (aHUS), autoimmune hemolytic anemia, chronic cold agglutinin disease, HELLP syndrome, and/or warm autoimmune hemolytic anemia. In some embodiments, a compound is administered to a subject suffering from, or at risk of, a complement-mediated disorder that affects the circulatory system. For example, in some embodiments, the disorder is thrombotic microangiopathy (TMA) or a vasculitis (e.g., IgA vasculitis) or other disorder associated with vessel inflammation, e.g., blood vessel and/or lymph vessel inflammation. In some embodiments, a vasculitis is polyarteritis nodosa, hypocomplementemic urticarial vasculitis, pulmonary vasculitis, Wegener's granulomatosis, giant cell arteritis, Churg-Strauss syndrome, microscopic polyangiitis, pauci-immune vasculitis, Henoch-Schonlein purpura, Takayasu's arteritis, Kawasaki disease, or Behcet's disease. In some embodiments, a disorder is TMA secondary to atypical hemolytic uremic syndrome. In some embodiments, a subject is positive for antineutrophil cytoplasmic antibody (ANCA).

### Eye Disorders

In some embodiments, a compound is administered to a subject for treatment of a complement-mediated eye disorder, such as macular degeneration (e.g., age-related macular degeneration (AMD) and Stargardt macular dystrophy), diabetic retinopathy, glaucoma, or uveitis (e.g., posterior uveitis or anterior uveitis). In some embodiments, a subject suffers from or is at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a compound, as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD.

In some embodiments, a subject has an eye disorder characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the eye disorder is Behcet's disease. In some embodiments, the eye disorder is an eye disorder characterized by optic nerve damage (e.g., optic nerve degeneration), such as glaucoma. Additional eye disorders include, e.g., retinitis pigmentosa, macular edema, Vogt-Koyangi-Harada syndrome, birdshot retino-chorioditis, sympathetic ophthalmia, ocular dicatricial pemphigoid, ocular pemphigus, nonartertic ischemic optic neuropathy, post-operative inflammation, and retinal vein occlusion.

### Nervous System Disorders

In some embodiments, a compound is used to treat a subject suffering from or at risk of a complement-mediated disorder that affects the nervous system, e.g., the central nervous system (CNS) and/or peripheral nervous system (PNS). Examples of such disorders include, e.g., a neurodegenerative disorder such as multiple sclerosis, other demyelinating diseases (e.g., neuromyelits optica or chronic inflammatory demyelinating polyneuropathy (CIDP)), amyotrophic lateral sclerosis, chronic pain, fibromyalgia, stroke, intracerebral hemorrhage, allergic neuritis, diabetic neuropathy, Huntington's disease, schizophrenia, Alzheimer's disease, Parkinson's disease, progressive supranuclear palsy, Lewy body dementia (i.e., dementia with Lewy bodies or Parkinson's disease dementia), frontotemporal dementia, progressive supranuclear palsy, corticobasal syndrome, Pick's disease, mild cognitive impairment, traumatic brain injury, traumatic spinal cord injury, multisystem atrophy, chronic traumatic encephalopathy, Creutzfeldt-Jakob disease, Guillain Barre Syndrome, and leptomeningeal metastasis. In some embodiments, a subject suffers from neuropathic pain, e.g., arising from lesions that involve the somatosensory pathways with damage to small fibres in peripheral nerves and/or to the spino-thalamocortical system in the CNS.

### Kidney Disorders

In some embodiments, a compound is used to treat a subject suffering from, or at risk of, a complement-mediated kidney disorder. Such disorders include, e.g., nephritis, e.g., glomerulonephritis, e.g., membranoproliferative glomerulonephritis (MPGN) (e.g., MPGN type I, MPGN type II, or MPGN type III), e.g., immune complex membranoproliferative glomerulonephritis (IC-MPGN). In some embodiments the disorder is IgA nephropathy (IgAN), primary membranous nephropathy, or diabetic nephropathy. In some embodiments, the disorder is polycystic kidney disease (PKD). In some embodiments, the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

### Respiratory Disorders

In some embodiments, a compound is used to treat a subject suffering from or at risk of a complement-mediated disorder respiratory disorder. In some embodiments, a subject is suffering from or at risk of acute respiratory distress syndrome. In some embodiments, a respiratory disease is, e.g., asthma (e.g., allergic asthma), emphysema, chronic inflammation, chronic obstructive pulmonary disease (COPD), pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, pulmonary embolisms and infarcts, dyspnea, hemoptysis, bronchoconstriction, or bronchiolitis obliterans.

### Musculoskeletal Disorders

In some embodiments, a compound is used to treat a subject suffering from, or at risk of, a complement-mediated disorder that affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a complement-mediated musculoskeletal disorder is myasthenia gravis.

### Transplantation

In some embodiments, a compound is used to protect a graft from complement-mediated damage. A graft can be contacted with a compound prior to, during, and/or after being transplanted, in various embodiments of the disclosure. In another embodiment, a compound is administered to a donor prior to removal of the graft. In some embodiments, a compound is administered to a recipient during and/or after the introduction of the graft. In some embodiments, a compound is administered to a recipient prior to the introduction of the graft. In some embodiments, a subject receives a compound after receiving the graft.

In some embodiments, a graft is or comprises a solid organ such as a kidney, liver, lung, pancreas, or heart. In some embodiments, a graft is or comprises bone, cartilage, fascia, tendon, ligament, cornea, sclera, pericardium, skin, heart valve, blood vessel, amniotic membrane, or dura mater. In some embodiments, a graft comprises multiple organs such as a heart-lung or pancreas-kidney graft. In some embodiments, a graft comprises less than a complete organ or tissue. For example, a graft may contain a portion of an organ or tissue, e.g., a liver lobe, section of blood vessel, skin flap, or heart valve. In some embodiments, a graft comprises a preparation comprising isolated cells or tissue fragments that have been isolated from their tissue of origin but retain at least some tissue architecture, e.g., pancreatic islets. In some embodiments, a preparation comprises isolated cells that are not attached to each other via connective tissue, e.g., hematopoietic stem cells or progenitor cells derived from peripheral and/or cord blood, or whole blood or any cell-containing blood product such as red blood cells (RBCs) or platelets.

In some embodiments, a graft is a xenograft (i.e., the donor and recipient are of different species), an autograft (i.e., a graft from one part of the body to another part of the body in the same individual), an isograft (i.e., the donor and recipient are genetically identical), or an allograft (i.e., the donor and recipient are genetically non-identical members of the same species).

### Ischemia/Reperfusion Injury

Ischemia-reperfusion (I/R) injury is an important cause of tissue damage following trauma and in other conditions associated with temporary disruption of blood flow such as myocardial infarction, stroke, severe infection, vascular disease, aneurysm repair, cardiopulmonary bypass, and transplantation. In the setting of trauma, systemic hypoxemia, hypotension, and local interruption of the blood supply resulting from contusions, compartment syndrome, and vascular injuries cause ischemia that damages metabolically active tissues. Restoration of the blood supply triggers an intense systemic inflammatory reaction. After reperfusion, all three major complement pathways are activated and, acting cooperatively or independently, are involved in I/R related adverse events affecting numerous organ systems.

In some embodiments a compound is administered to a subject who has recently (e.g., within the preceding 2, 4, 8, 12, 24, or 48 hours) experienced trauma, e.g., trauma that puts the subject at risk of I/R injury, e.g., due to systemic hypoxemia, hypotension, and/or local interruption of the blood supply. In some embodiments, a compound may be administered intravascularly, optionally into a blood vessel that supplies an injured body part or directly to the body part. In some embodiments, the subject suffers from spinal cord injury, traumatic brain injury, burn, and/or hemorrhagic shock.

In some embodiments, a compound is administered to a subject prior to, during, or after a surgical procedure, e.g., a surgical procedure that is expected to temporarily disrupt blood flow to a tissue, organ, or portion of the body. Examples of such procedures include cardiopulmonary bypass, angioplasty, heart valve repair/replacement, aneurysm repair, or other vascular surgeries. A compound may be administered prior to, after, and/or during an overlapping time period with the surgical procedure.

In some embodiments, a compound is administered to a subject who has suffered an MI, thromboembolic stroke, deep vein thrombosis, or pulmonary embolism. A compound may be administered in combination with a thrombolytic agent such as tissue plasminogen activator (tPA) (e.g., alteplase (Activase), reteplase (Retavase), tenecteplase (TNKase)), anistreplase (Eminase), streptokinase (Kabikinase, Streptase), or urokinase (Abbokinase). In some embodiments, a compound may be administered prior to, after, and/or during an overlapping time period with the thrombolytic agent.

### Other Disorders

In some embodiments, a compound is used to treat a subject suffering from, or at risk of, a complement-mediated disorder that affects the integumentary system. Examples of such disorders include, e.g., atopic dermatitis, psoriasis, pemphigoid, pemphigus, systemic lupus erythematosus, dermatomyositis, scleroderma, sclerodermatomyositis, Sjögren syndrome, and chronic urticaria.

In some embodiments, a compound is used to treat a subject suffering from, or at risk of, a complement-mediated disorder that affects the gastrointestinal system, e.g., inflammatory bowel disease, e.g., Crohn's disease or ulcerative colitis.

In some embodiments, a compound is used to treat a subject suffering from, or at risk of, a complement-mediated inflammatory disorder, such as rhinosinusitis or myocarditis.

In some embodiments, a compound is used to treat a subject suffering from, or at risk of, thyroiditis (e.g., Hashimoto's thyroiditis, Graves' disease, post-partum thyroiditis), hepatitis (e.g., hepatitis C), pancreatitis, panniculitis, or MYH9-related disorders.

In some embodiments, a compound is used to treat interleukin-2 induced toxicity during IL-2 therapy, myocardial infarction, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, liver fibrosis, fibrogenic dust diseases, nasal polyposis, parasitic diseases, Goodpasture's Syndrome, immune complex-associated inflammation, antiphospholipid syndrome, cancer, periodontitis, gingivitis, or obesity.

In some embodiments, a complement-mediated condition, disorder or disease is complement activation secondary to administration of another therapeutic or diagnostic agent. For example, in some embodiments, a complement-mediated condition, disorder or disease is complement activation secondary to gene therapy (e.g., gene therapy with a viral vector such as an adeno-associated virus (AAV), adenovirus, or lentivirus vector) or complement activation secondary to cell therapy). In some embodiments, a subject suffers from TMA secondary to hematopoietic stem cell transplant (HSCT-TMA). In some embodiments, a subject suffers from drug-induced TMA. In some embodiments, administration of a compound described herein prior to and/or following administration of another therapeutic agent may increase efficacy and/or safety of said therapeutic agent.

### V. Combination Therapy

In some embodiments, provided technologies comprise administering a compound, e.g., a compound of formula I or a pharmaceutically acceptable salt thereof, in combination with another therapy or therapeutic agent. In some embodiments, provided technologies comprise administering a compound, e.g., a compound of formula I or a pharmaceutically acceptable salt thereof, in combination with one or more additional complement inhibitors. In some embodiments, a provided compound or composition is administered to a subject already receiving therapy with another complement inhibitor. In some embodiments, another complement inhibitor is administered to a subject receiving a provided compound, e.g., a compound of formula I or a pharmaceutically acceptable salt thereof, or a composition thereof. In some embodiments, both a provided compound, e.g., a compound of formula I or a pharmaceutically acceptable salt thereof and another complement inhibitor are administered to a subject.

In some embodiments, administration of a provided compound, e.g., a compound of formula I or a pharmaceutically acceptable salt thereof may allow for administering a reduced dosing regimen of (e.g., involving a smaller amount in an individual dose, reduced frequency of dosing, reduced number of doses, and/or reduced overall exposure to) a second therapeutic agent, e.g., a complement inhibitor, as compared to administration of a second therapeutic agent, e.g., a complement inhibitor, as single therapy. Without wishing to be bound by any theory, in some embodiments a reduced dosing regimen of a second therapeutic agent, e.g., a complement inhibitor, may avoid one or more undesired adverse effects that could otherwise result.

In some aspects, administration of a provided compound, e.g., a compound of formula I or a pharmaceutically acceptable salt thereof in combination with a second therapeutic agent, e.g., a complement inhibitor can reduce the level of C3 activity in a subject's blood sufficiently such that a reduced dosing regimen of a provided compound and/or the second therapeutic agent is required to achieve a desired degree of complement inhibition.

In some embodiments such a reduced dose can be administered in a smaller volume, or using a lower concentration, or using a longer dosing interval, or any combination of the foregoing, as compared to administration of a provided compound, e.g., a compound of formula I or a pharmaceutically acceptable salt thereof or a second therapeutic agent, e.g., a complement inhibitor as single therapy.

### A. Additional Complement Inhibitors

In some aspects, methods of the present disclosure involve administering a compound described herein, alone or in combination with one or more additional complement inhibitors. In some embodiments, a compound is administered to a subject already receiving therapy with another complement inhibitor; in some embodiments, another complement inhibitor is administered to a subject receiving a compound. In some embodiments, both a compound and another complement inhibitor are administered to the subject.

In some embodiments administration of a compound may allow for administering a reduced dosing regimen of (e.g., involving a smaller amount in an individual dose, reduced frequency of dosing, reduced number of doses, and/or reduced overall exposure to) a second complement inhibitor, as compared to administration of a second complement inhibitor as single therapy. Without wishing to be bound by any theory, in some embodiments a reduced dosing regimen of a second complement inhibitor may avoid one or more undesired adverse effects that could otherwise result.

In some aspects, administration of a compound in combination with a second complement inhibitor can reduce the amount of C3 in the subject's blood sufficiently such that a reduced dosing regimen of a compound and/or the second complement inhibitor is required to achieve a desired degree of complement inhibition.

In some aspects, administration of a compound in combination with a second complement inhibitor can reduce the amount of C3 in the subject's blood sufficiently such that a reduced dosing regimen of a compound and/or the second complement inhibitor is required to achieve a desired level of, or a desired amount of improvement in, one or more signs, symptoms, biomarkers, or outcome measures, of a complement-mediated disorder.

In some embodiments such a reduced dose can be administered in a smaller volume, or using a lower concentration, or using a longer dosing interval, or any combination of the foregoing, as compared to administration of a compound or a second complement inhibitor as single therapy.

Any complement inhibitor, e.g., a complement inhibitor known in the art, can be administered in combination with a compound described herein. In some embodiments, a complement inhibitor is compstatin or a compstatin analog.

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His -Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7 (3): 619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32 (Pt 1): 28-32, 2004, Mallik, B., et al., J. Med. Chem., 274-286, 2005; Katragadda, M., et al. J. Med. Chem., 49:4616-4622, 2006; WO2007062249 (PCT/US2006/045539); WO2007044668 (PCT/US2006/039397), WO/2009/046198 (PCT/US2008/078593); WO/2010/127336 (PCT/US2010/033345).

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure.

TABLE 1

| | | SEQ | Activity |
|---|---|---|---|
| | | ID | over |
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3 × more |
| Ac-V4Y/H9A | Ac-ICVYQDWGAHRCT-CONH2 | 10 | 14 × more |
| Ac-V4W/H9A —OH | Ac-ICVWQDWGAHRCT-COOH | 11 | 27 × more |
| Ac-V4W/H9A | Ac-ICVWQDWGAHRCT-CONH2 | 12 | 45 × more |
| Ac-V4W/H9A/T13dT —OH | Ac-ICVWQDWGAHRCdT-COOH | 13 | 55 × more |
| Ac-V4(2-Nal)/H9A | Ac-ICV(2-Nal)QDWGAHRCT- | 14 | 99 × more |
| | CONH2 | | |
| Ac V4(2-Nal)/H9A —OH | Ac-ICV(2-Nal)QDWGAHRCT- | 15 | 38 × more |
| | COOH | | |
| Ac V4(1-Nal)/H9A —OH | Ac-ICV(1-Nal)QDWGAHRCT- | 16 | 30 × more |
| | COOH | | |
| Ac-V42IgI/H9A | Ac-ICV(2-IgI)QDWGAHRCT- | 17 | 39 × more |
| | CONH2 | | |
| Ac-V42IgI/H9A —OH | Ac-ICV(2-IgI)QDWGAHRCT- | 18 | 37 × more |
| | COOH | | |
| Ac-V4Dht/H9A —OH | Ac-ICVDhtQDWGAHRCT-COOH | 19 | 5 × more |
| Ac-V4(Bpa)/H9A —OH | Ac-ICV(Bpa)QDWGAHRCT-COOH | 20 | 49 × more |
| Ac-V4(Bpa)/H9A | Ac-ICV(Bpa)QDWGAHRCT- | 21 | 86 × more |
| | CONH2 | | |
| Ac-V4(Bta)/H9A —OH | Ac-ICV(Bta)QDWGAHRCT-COOH | 22 | 65 × more |
| Ac-V4(Bta)/H9A | Ac-ICV(Bta)QDWGAHRCT-CONH2 | 23 | 64 × more |
| Ac-V4W/H9(2-Abu) | Ac-ICVWQDWG(2-Abu)HRCT- | 24 | 64 × more |
| | CONH2 | | |
| +G/V4W/H9A + AN —OH | H-GICVWQDWGAHRCTAN- | 25 | 38 × more |
| | COOH | | |
| Ac-V4(5fW)/H9A | Ac-ICV(5fW)QDWGAHRCT- | 26 | 31 × more |
| | CONH₂ | | |
| Ac-V4(5-MeW)/H9A | Ac-ICV(5-methyl- | 27 | 67 × more |
| | W)QDWGAHRCT- CONH₂ | | |
| Ac-V4(1-MeW)/H9A | Ac-ICV(1-methyl- | 28 | 264 × more |
| | W)QDWGAHRCT- CONH₂ | | |
| Ac-V4W/W7(5fW)/H9A | Ac-ICVWQD(5fW)GAHRCT- | 29 | 121 × more |
| | CONH₂ | | |
| Ac-V4(5fW)/W7(5fW)/H9A | Ac-ICV(5fW)QD(5fW)GAHRCT- | 30 | NA |
| | CONH₂ | | |
| Ac-V4(5- | Ac-ICV(5-methyl- | 31 | NA |
| MeW)/W7(5fW)H9A | W)QD(5fW)GAHRCT- CONH₂ | | |
| Ac- | Ac-ICV(1-methyl- | 32 | 264 × more |
| V4(1McW)/W7(5fW)/H9A | W)QD(5fW)GAHRCT-CONH₂ | | |
| +G/V4(6fW)/W7(6fW)H9A + | H-GICV(6fW)QD(6fW)GAHRCTN- | 33 | 126 × more |
| N—OH | COOH | | |
| Ac-V4(1-formyl-W)/H9A | Ac-ICV(1-formyl- | 34 | 264 × more |
| | W)QDWGAHRCT-CONH₂ | | |
| Ac-V4(5-methoxy-W)/H9A | Ac-ICV(1-methyoxy- | 35 | 76 × more |
| | W)QDWGAHRCT-CONH₂ | | |
| G/V4(5f- | H-GICV(5fW)QD(5fW)GAHRCTN- | 36 | 112 × more |
| W)/W7(5fW)/H9A + N—OH | COOH | | |

NA = not available

In certain embodiments of the compositions and methods of the disclosure, the compstatin analog has a sequence selected from sequences 9-36. In one embodiment, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13. In some embodiments, a compstatin analog (e.g., any one of the compstatin analogs listed in Table 1) comprises an isoleucine at position corresponding to position 3 of SEQ ID NO: 8, cither instead of or in addition to one or more substitutions described herein. For example, in some embodiments, a compstatin analog comprises or consists of the sequence of any one of SEQ ID NOs: 8-36, where position 3 is an isoleucine. In some embodiments, a compstatin analog comprises or consists of the sequence of any one of SEQ ID NOs: 25, 33, or 36, where position 4 is an isoleucine. Additional compstatin analogs are described in, e.g., WO2019/166411.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3rd ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo (ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

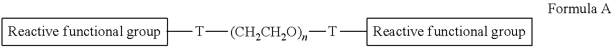

wherein each T and “Reactive functional group” is independently as defined below, and described in classes and subclasses herein, and n is as defined above and described in classes and subclasses herein.
Each T is independently a covalent bond or a C₁₋₁₂ straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and
each R^{x} is independently hydrogen or C₁₋₆ aliphatic.
The Reactive functional group has the structure —COO—NHS.

Exemplary bifunctional PEGs of formula A include:

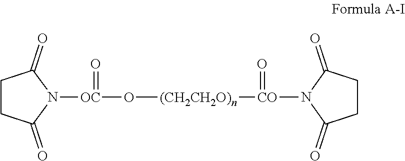

In some embodiments, a functional group (for example, an amine, hydroxyl, or thiol group) on a compstatin analog is reacted with a PEG-containing compound having a “reactive functional group” as described herein, to generate such conjugates. By way of example, Formula A-I can form compstatin analog conjugates having the structure:

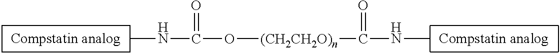

wherein,

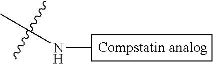 [SMILES: *NC]

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

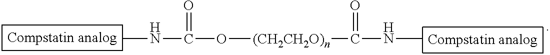

In some embodiments of formulae and embodiments described herein,

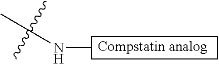 [SMILES: *NC]

represents point of attachment of a lysine side chain group in a compstatin analog having the structure:

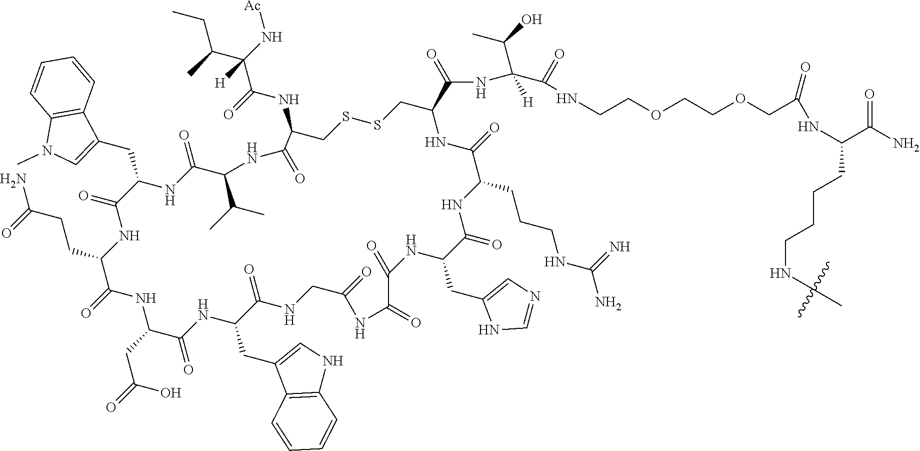 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)NC(=O)C(=O)N[C@@H](Cc2cnc[nH]2)C(=O)N[C@@H](CCCNC(=N)N)C(=O)N1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)ₙCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the a-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)ₙCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)ₙCH₂C(═O)OH linker introduces a —NH(CH₂CH₂O)ₙCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the a-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the s-amino group of said lysine residue. In some embodiments, the C-terminus of the lysine residue is modified. In some embodiments, the C-terminus of the lysinc residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted below:

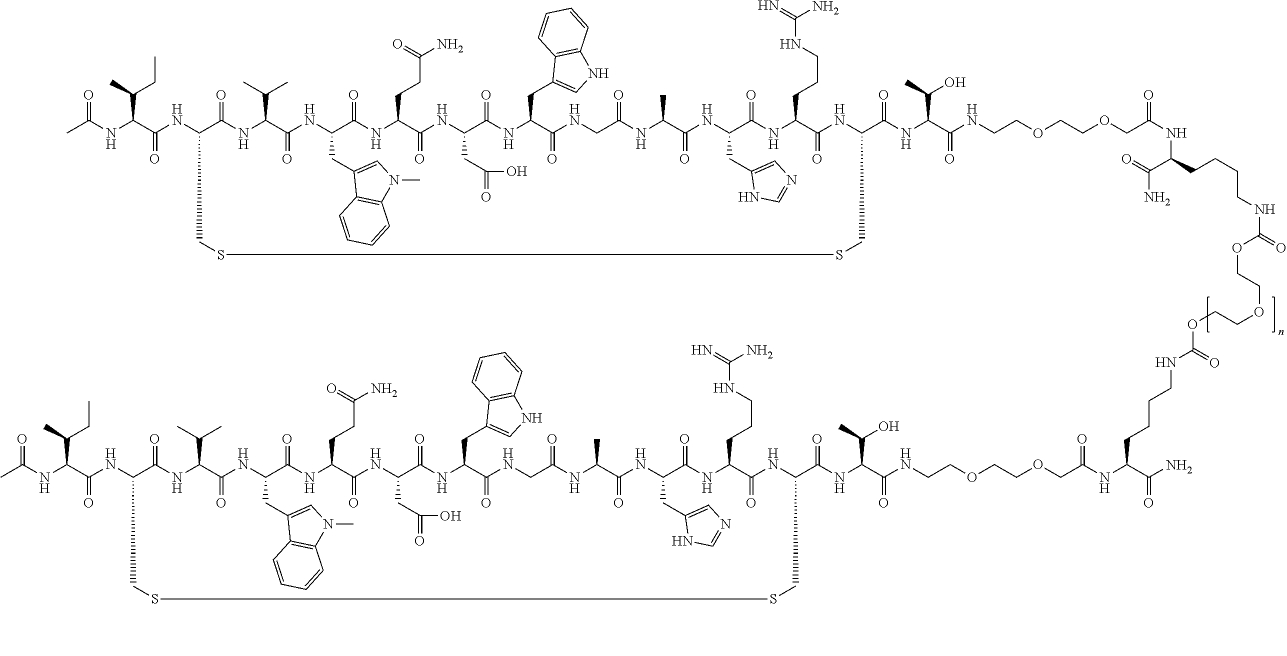

It will be understood that the PEG moiety may have a variety of different molecular weights or average molecular weights in various embodiments, as described herein. In certain embodiments, a compstatin analog is pegcetacoplan (“APL-2”), having the structure above with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2->12)-(disulfide); or O,O′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6,15}-carbonyl]polyethylene glycol (n=800-1100). Additional compstatin analogs are described in, e.g., WO 2012/155107 and WO 2014/078731.

In some embodiments, a compstatin analog described herein is administered twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, a composition comprising a provided compound is administered to a subject in combination with a compstatin analog, such that the compstatin analog and/or the composition is administered less frequently and/or at a lower dosage. In some embodiments, a composition comprising a provided compound is administered to a subject in combination with a compstatin analog, such that the compstatin analog is administered once a week, once every 2 weeks, once a month, once every 2 months, 3 months, 4 months, 5 months, or longer, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg.

In some embodiments, a complement inhibitor is an antibody, e.g., an anti-C3 and/or anti-C5 antibody, or a fragment thereof. In some embodiments, an antibody fragment may be used to inhibit C3 or C5 activation. The fragmented anti-C3 or anti-C5 antibody may be Fab′, Fab′(2), Fv, or single chain Fv. In some embodiments, the anti-C3 or anti-C5 antibody is monoclonal. In some embodiments, the anti-C3 or anti-C5 antibody is polyclonal. In some embodiments, the anti-C3 or anti-C5 antibody is de-immunized. In some embodiments the anti-C3 or anti-C5 antibody is a fully human monoclonal antibody. In some embodiments, the anti-C5 antibody is eculizumab. In some embodiments, a complement inhibitor is an antibody, e.g., an anti-C3 and/or anti-C5 antibody, or a fragment thereof.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943). Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

A variety of other complement inhibitors can also be used in various embodiments of the disclosure. In some embodiments, the complement inhibitor is a naturally occurring mammalian complement regulatory protein or a fragment or derivative thereof. For example, the complement regulatory protein may be CR1, DAF, MCP, CFH, or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can also be used. For example the compounds known as TP10 or TP20 (Avant Therapeutics) can be used. C1 inhibitor (C1-INH) can also be used. In some embodiments a soluble complement control protein, e.g., CFH, is used.

Inhibitors of C1s can also be used. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 73) or KTISKAKGQPREPQVYT (SEQ ID NO: 74) or a peptide having significant sequence identity and/or three-dimensional structural similarity thereto. In some embobiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

### B. Administration

In some embodiments, a provided compound, e.g., a compound of formula I or a pharmaceutically acceptable salt thereof, administered in an amount that inhibits plasma complement activity by an average of no more than 95%, optionally between 50% and 95%, as measured using an alternative pathway assay, a classical pathway assay, or both, may be administered in combination with a second complement inhibitor, e.g., a long-acting compstatin analog (LACA). In some embodiments, a LACA administered in an amount that inhibits plasma complement activity by an average of no more than 95%, optionally between 50% and 95%, as measured using an alternative pathway assay, a classical pathway assay, or both, may be administered in combination with a provided compound. In some embodiments, the assay is a hemolysis assay. In some embodiments, a provided compound administered in an amount effective to reduce the steady state plasma level of C3 by between 30% and 95% on average, e.g., between 50% and 95%, e.g., between 50% and 60%, between 60% and 70%, between 70% and 80%, or between 80% and 90%, on average, may be administered in combination with a LACA. In some embodiments, a provided compound may be administered in amounts that are effective to reduce the steady state plasma level of C3 by more than 95% but still do not achieve a desired efficacy. Combined administration with the LACA allows such efficacy to be achieved. In some embodiments, a provided compound may be administered at between 80% and 100% of its maximum tolerated dose. In some embodiments, combined administration with a LACA allows the use of smaller doses of a provided compound than those required to achieve a desired level of efficacy. In some embodiments, a provided compound may be administered at less than 50%, 60%, 70%, or 80% of its maximum tolerated dose.

In some embodiments, a provided compound and a second therapeutic agent, e.g., a complement inhibitor may be administered once daily, weekly, every 2, 3, or 4 weeks, or even at longer intervals. In some embodiments, a provided compound is administered to a subject once, e.g., as a single injection or as a single infusion over time (e.g., over 5, 10, 15, 20, 30, 40, 50, 60, 90, 120 minutes, or longer), and a second complement inhibitor is administered once a week, twice a week, every three days, thrice a week, every other day, once every 2 weeks, every 3 weeks, once a month, every 6 weeks, once every 2 months, 3 months, 4 months, 5 months, or longer. In some embodiments, a provided compound is administered to a subject twice, e.g., as two injections (e.g., 2, 4, 6, 8, 10, or 12 hours apart) or as two infusions (e.g., 2, 4, 6, 8, 10, or 12 hours apart), and a second complement inhibitor is administered once a week, twice a week, every three days, thrice a week, every other day, once every 2 weeks, every 3 weeks, once a month, every 6 weeks, once every 2 months, 3 months, 4 months, 5 months, or longer. In some embodiments, a provided compound and a second therapeutic agent, e.g., a complement inhibitor may be administered according to a dosing regimen that includes a second complement inhibitor administered once a week, twice a week, every three days, thrice a week, every other day, once every 2 weeks, every 3 weeks, once a month, every 6 weeks, once every 2 months, 3 months, 4 months, 5 months, or longer; and (i) a single administration or an initial administration of a provided compound that is once daily, weekly, every 2, 3, or 4 weeks, or even at longer intervals; followed by (ii) a period of no administration of a provided compound of, e.g., 1, 2, 3, 4, 5, 6, 8, or 10 months, or 1, 2, 3, 4, 5, 6, 7, 8, 9, or 10 years. In some embodiments, a subject is monitored before and/or following treatment for level of C3 expression and/or activity, e.g., as measured using an alternative pathway assay, a classical pathway assay, or both. Suitable assays are known in the art and include, e.g., a hemolysis assay. In some embodiments, a subject is treated (e.g., with a provided compound and/or with a second therapeutic agent, e.g., a complement inhibitor), or is retreated (e.g., with a provided compound and/or with a second therapeutic agent, e.g., a complement inhibitor), if a measured level of C3 expression and/or activity is more than 10%, 20%, 30%, 40%, 50%, 100%, 200%, or more, relative to measured level of C3 expression and/or activity in a control subject.

In some embodiments it may be desirable to administer a provided compound and a second therapeutic agent, e.g., a complement inhibitor according to the same dosing schedule (e.g., once per day, every other day, or once per week), while in other embodiments different dosing schedules may be used. In some embodiments, a provided compound and a second therapeutic agent, e.g., a complement inhibitor are administered subcutaneously. In some embodiments, a provided compound may be administered intravenously.

In some embodiments, a provided compound achieves therapeutically useful levels of complement inhibition when administered as sole complement inhibiting therapy once or twice daily, e.g., subcutaneously. In some embodiments, a provided compound achieves therapeutically useful levels of C3 inhibition when administered as sole C3 inhibiting therapy once or twice daily, e.g., subcutancously. In some embodiments, a provided compound may be administered in a lower total amount (as measured over a relevant time period such as a month) when administered in combination with a second therapeutic agent, e.g., a complement inhibitor that inhibits C3 expression and/or activity. In some embodiments, a total amount administered may be lower by a factor of at least 1.5, e.g., lower by a factor of between 1.5 and 5, between 5 and 10, or between 10 and 20, over a relevant time period such as a month. In some embodiments, a provided compound may be administered in smaller daily doses as compared with the doses that would be used if a provided compound was administered as sole complement inhibitor therapy or as sole C3 inhibiting therapy. In some embodiments, a provided compound may be administered using a longer dosing interval as compared with the dosing interval that would be used if a provided compound was administered as sole complement inhibitor therapy or as sole C3 inhibiting therapy. For example, in some embodiments, a provided compound that would typically be administered daily to achieve a desired effect may instead be administered every other day, every 3 days, or weekly, to achieve substantially the same effect. In some embodiments, a provided compound may be administered using both lower individual doses and a longer dosing interval when administered in combination with a LACA than when administered as sole complement inhibitor therapy or as sole C3 inhibiting therapy.

Certain LACAs comprising a PEG of about 40 kD demonstrate pharmacological activity when administered subcutaneously at daily doses of 180 mg and 270 mg, with 270 mg/day being particularly effective. In some embodiments, such a LACA, when administered in combination with a provided compound may be administered at a reduced dose, e.g., a dose that is lower by a factor of at least 1.5, e.g., lower by a factor of between 1.5 and 5, between 5 and 10, or between 10 and 20, relative to administration of LACA alone. In some embodiments, for example, the dose may be between about 9 mg/day and about 150 mg/day, e.g., between about 9 mg/day and about 20 mg/day, between about 20 mg/day and about 50 mg/day, between about 50 mg/day and 100 mg/day, between about 100 mg/day and about 150 mg/day, and in at least some embodiments achieves at least equivalent efficacy to a 180 mg/day dose or in some embodiments a 270 mg/day dose. In some embodiments the dose may be between about 150 mg/day and about 200 mg/day, and in at least some embodiments achieves at least equivalent efficacy to a 270 mg/day dose. In some embodiments the dose is 10 mg/day-20 mg/day, 20 mg/day-30 mg/day, 30 mg/day-40 mg/day, 40 mg/day-50 mg/day, 50 mg/day-60 mg/day, 60 mg/day-70 mg/day, 70 mg/day-80 mg/day, 80 mg/day-90 mg/day, 90 mg/day-100 mg/day, 100 mg/day-110 mg/day, 110 mg/day-120 mg/day, 120 mg/day-130 mg/day, 130 mg/day-140 mg/day, 140 mg/day-150 mg/day, 150 mg/day-160 mg/day, 160 mg/day-170 mg/day, 170 mg/day-180 mg/day, 180 mg/day-190 mg/day, or 190 mg/day-200 mg/day. In some embodiments the dose is 200 mg/day-210 mg/day, 210 mg/day-220 mg/day, 220 mg/day-230 mg/day, 230 mg/day-240 mg/day, or 240 mg/day-250 mg/day. In some embodiments the dose of LACA is administered as a single daily dose, e.g., subcutaneously. In some embodiments a dose of LACA is administered as a single weekly dose, e.g., subcutaneously.

In some aspects, a reduced dose of LACA may be administered in a smaller volume and/or at a reduced concentration, when administered in combination with a provided compound, relative to administration of LACA alone. For example, if the dose is reduced by a factor of 10, the volume could also be reduced by a factor of 10 while keeping the concentration the same. Alternately, the concentration could be reduced by a factor of 10 while keeping the volume the same. Alternately both the concentration and volume may be reduced. In certain embodiments the volume of an individual dose is about 0.8 ml or less, e.g., 0.5 ml or less, e.g., between 0.02 ml and 0.5 ml, e.g., 0.1 ml, 0.2 ml, 0.3 ml, 0.4 ml, or 0.5 ml. In certain embodiments the concentration is below about 100 mg/ml. For example, the concentration may be 10 mg/ml-20 mg/ml, 20 mg/ml-30 mg/ml, 30 mg/ml-40 mg/ml, 40 mg/ml-50 mg/ml, 50 mg/ml-60 mg/ml, 60 mg/ml-70 mg/ml, 70 mg/ml-80 mg/ml, 80 mg/ml-90 mg/ml, or 90 mg/ml-100 mg/ml. The volume and concentration can be selected to deliver a desired amount. For example, in an exemplary embodiment a dose of 40 mg is administered in a volume of 0.5 ml at a concentration of 80 mg/ml. In another exemplary embodiment a dose of 60 mg is administered in a volume of 0.6 ml at a concentration of 100 mg/ml. In some embodiments a 28, 29, 30, or 31 gauge needle may be used to administer the LACA, a provided compound, or both.

In some embodiments, a LACA described herein is administered twice weekly or every 3 days or thrice weekly, at a dosage of about 10 mg to about 10 g, e.g., about 10 mg to about 20 mg, e.g., about 20 mg to about 40 mg, e.g., about 40 mg to about 60 mg, e.g., about 60 mg to about 80 mg, e.g., about 80 mg to about 100 mg, e.g., about 100 mg to about 120 mg, e.g., about 120 mg to about 140 mg, e.g., about 140 mg to about 160 mg, e.g., about 160 mg to about 180 mg, e.g., about 180 mg to about 200 mg, e.g., about 200 mg to about 220 mg, e.g., about 220 mg to about 240 mg, e.g., about 240 mg to about 260 mg, e.g., about 260 mg to about 280 mg, e.g., about 280 mg to about 300 mg, e.g., about 300 mg to about 320 mg, e.g., about 320 mg to about 340 mg, e.g., about 340 mg to about 360 mg, e.g., about 360 mg to about 380 mg, e.g., about 380 mg to about 400 mg, e.g., about 400 mg to about 420 mg, e.g., about 420 mg to about 440 mg, e.g., about 440 mg to about 460 mg, e.g., about 460 mg to about 480 mg, e.g., about 480 mg to about 500 mg, e.g., about 500 mg to about 520 mg, e.g., about 520 mg to about 540 mg, e.g., about 540 mg to about 560 mg, e.g., about 560 mg to about 580 mg, e.g., about 580 mg to about 600 mg, e.g., about 100 mg to about 200 mg, e.g., about 545 mg to about 1690 mg, e.g., about 585 mg to about 2510 mg, e.g., about 630 mg to about 930 mg, e.g., about 795 mg to about 885 mg, e.g., about 900 mg to about 1395 mg, e.g., about 990 mg to about 1215 mg, e.g., about 1215 mg to about 1395 mg, e.g., about 2160 mg to about 2520 mg, e.g., about 2520 mg to about 2880 mg, e.g., about 2880 mg to about 3240 mg, e.g., about 3240 mg to about 3600 mg, e.g., about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1080 mg to about 5040 mg, about 5000 mg-1.0 g, e.g., about 1.0-2.0 g, or more, e.g., up to about 4.0-5.0 g, or up to about 6.0 g, or up to about 10.0 g, e.g., between about 4.0 g and about 6.0 g, e.g., between about 4.5 g and about 5.5 g, e.g., about 5.0 g, e.g., between about 5.0 g and about 7.0 g, e.g., about 5.0 g, about 5.5 g, about 6.0 g, about 6.5 g, or about 7.0 g, e.g., between about 8.0 g and about 10.0 g, e.g., about 8.0 g, about 8.5 g, about 9.0 g, about 9.5 g, or about 10.0 g, e.g., between about 4.0 g and about 10.0 g, e.g., about 5.0 g, about 6.0 g, about 7.0 g, about 8.0 g, about 8.5 g, about 9.0 g, about 9.5 g, or about 10.0 g, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, a LACA described herein is administered to a subject in need thereof at about 10 mg to about 10 g (e.g., about 10 mg to about 600 mg, about 600 mg to about 1200 mg, about 1250 mg to about 2000 mg, about 2000 mg to about 2500 mg, about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg, about 2500-5000 mg, about 5000 mg-1.0 g, about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g) or more.

In some embodiments, a LACA described herein is administered as two or more doses. In some embodiments, a first dose (e.g., a loading dose) and a second dose (e.g., a maintenance dose) are administered. In some embodiments, the first dose and the second dose comprise the same amount of the LACA. In some embodiments, the first dose and the second dose comprise different amounts of the LACA.

In some embodiments, the first dose comprises about 10 mg to about 10 g of the LACA (e.g., about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg, about 2500-5000 mg, about 5000 mg-1.0 g, about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g) and the second dose comprises about 10 mg to about 10 g of the LACA (e.g., about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg, about 2500-5000 mg, about 5000 mg-1.0 g, about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

In some embodiments, a provided compound is administered to a subject in combination with a LACA, such that the LACA and/or a provided compound is administered less frequently and/or at a lower dosage, relative to administration of a LACA alone or relative to administration of the provided compound alone.

In some embodiments, a provided compound is administered to a subject in combination with a LACA, e.g., a LACA comprising a PEG of about 40 kD, such that the LACA is administered once a week, twice a week, every three days, thrice a week, every other day, once every 2 weeks, every 3 weeks, once a month, every 6 weeks, once every 2 months, 3 months, 4 months, 5 months, or longer, at a dosage described herein (for e.g., at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

In some embodiments, a provided compound is administered to a subject once, e.g., as a single injection or as a single infusion over time (e.g., over 5, 10, 15, 20, 30, 40, 50, 60, 90, 120 minutes, or longer), in combination with a LACA, e.g., a LACA of comprising a PEG of about 40 kD, such that the LACA is administered once a week, twice a week, every three days, thrice a week, every other day, once every 2 weeks, once every 3 weeks, once a month, once every 6 weeks, once every 2 months, 3 months, 4 months, 5 months, or longer, at a dosage described herein (for e.g., at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

In some embodiments, a provided compound is administered to a subject twice, e.g., as two injections (e.g., 2, 4, 6, 8, 10, or 12 hours apart) or as two infusions (e.g., 2, 4, 6, 8, 10, or 12 hours apart), in combination with a LACA, e.g., a LACA comprising a PEG of about 40 kD, such that the LACA is administered once a week, twice a week, every three days, thrice a week, every other day, once every 2 weeks, once every 3 weeks, once a month, once every 6 weeks, once every 2 months, 3 months, 4 months, 5 months, or longer, at a dosage described herein (for e.g., at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

While doses of 250 mg/day or less are of particular interest for administration, e.g., SC administration, of a LACA in combination with a provided compound that inhibits C3 activity, the present disclosure also contemplates administering doses of more than 250 mg/day in combination with a provided compound that inhibits C3 activity, e.g., doses of 250 mg/day-300 mg/day, 300 mg/day-400 mg/day, or 400 mg/day-500 mg/day. In certain embodiments such a dose may be administered weekly, twice a week, or every 3 days. In certain embodiments, LACA doses in combination with a provided compound that inhibits C3 activity may be administered twice weekly or every 3 days, at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg.

While the present disclosure particularly contemplates embodiments in which a LACA having a terminal half-life of at least 2, 3, 4, or more days when administered IV or SC to a primate, e.g., LACAs comprising a clearance reducing moiety as described herein, is administered in combination with a compound that inhibits C3 activity, it is contemplated in certain embodiments that combined administration with such a provided compound may also be useful for compstatin analogs that have shorter half-lives and/or that lack a clearance reducing moiety. Such compstatin analogs may be administered in 1 or 2 doses per day.

In some embodiments efficacy of a particular agent or combination of agents may be measured by lactate dehydrogenase (LDH) level in a patient suffering from a complement-mediated hemolytic disorder such as PNH. As will be appreciated by those of ordinary skill in the art, complement-mediated hemolysis results in release of LDH, which can result in an abnormally elevated plasma LDH level. Thus in some aspects, efficacy of a complement inhibitor may be evidenced in a subject suffering from a complement-mediated hemolytic disorder by a decrease in plasma LDH level, e.g., to within nomal limits. Other indicators of efficacy in a subject suffering from a complement-mediated hemolytic disorder may include, e.g., a reduction in reticulocyte count in a subject who has an elevated reticulocyte count (e.g., normalization of reticulocyte count), a reduced need for transfusions, an increased hemoglobin level, stabilization of hemoglobuin level without need for transfusions in a subject who had required multiple transfusions in the previous year. In some embodiments efficacy may be measured by a classical or alternative pathway complement assay, which may be a hemolysis assay.

In some embodiments a composition as described herein, and e.g., a composition comprising a LACA described herein, is administered using a device that delivers a dose of a pharmaceutical composition by injection, in some embodiments in an at least partly automated fashion upon activation. Such a device is referred to in the art as a “pen” or “autoinjector”, and these terms are used interchangeably herein. In general, a pen or autoinjector allows for injecting a dose of pharmaceutical composition contained in a cartridge, reservoir, or syringe through an automatically or manually inserted hypodermic needle(s) or through a high velocity jet. It may be designed for administration of a single dose or multiple doses.

In some embodiments, such a pen or autoinjector is utilized for intramuscular and/or subcutaneous injection. In accordance with the present disclosure, a pen or other autoinjector may be particularly useful for embodiments that utilize subcutaneous injection. Pens are typically devices that contain (or can be loaded with) a medication in a self-contained cartridge or reservoir and to which a needle can be attached.

In some embodiments, such injection is achieved by use of a pen (e.g., that may have been pre-loaded with an appropriate dose or volume). Pens can be durable (and reusable) or disposable. A durable pen typically uses a replaceable cartridge, which is disposed of when empty, and a new one is inserted in the pen. A disposable pen typically comes pre-filled with a medication in a cartridge or reservoir. When the cartridge or reservoir is empty, the pen can be discarded. The cartridge or reservoir may contain a single dose or multiple doses. To use a pen, a needle can be attached to the pen and inserted into the skin. Typically, a button can be pushed to administer a dose though in some embodiments other activation methods may be used. In some embodiments, an autoinjector may comprise a spring-loaded syringe, though one of ordinary skill in the art will appreciate that a variety of technologies are available to afford automatic administration. In some embodiments, by pressing a button or otherwise activating the device, the needle can be automatically inserted, and the medication can be delivered. In some embodiments, an autoinjector may be designed to insert the needle automatically and/or accurately to a desired depth in the subcutaneous tissue. A pen or autoinjector may comprise means such as a dial that allows a user to select or adjust a dose or injection depth.

In some embodiments, a composition as described herein, e.g., a LACA described herein, is administered using a device comprising a dual chamber syringe. Dry drug (e.g., lyophilized) is contained in one chamber. The second chamber contains a suitable pharmaceutically acceptable carrier. In order to use the device, the drug is first reconstituted by mixing the contents of the chambers. This can be accomplished in various ways, as is known in the art. In some embodiments, pushing the plunger causes the contents of the chambers to mix, e.g., by transferring the carrier into the chamber containing the lyophilized drug.

Thus a variety of drug delivery devices comprising a composition as described herein (e.g., a LACA described herein) may be provided e.g., prefilled syringes, dual chamber syringes, durable and/or disposable pens, and cartridges suitable for use with a pen. Such devices may contain one or more doses (e.g., one or more of any of the dose amounts described herein).

In certain embodiments a LACA may be administered, e.g., subcutaneously, using a drug delivery device (sometimes referred to simply as a “delivery device”) that comprises a pump to introduce a liquid composition comprising the LACA into the subject's body. As will be appreciated, a pump may be any device that moves fluids by mechanical action as opposed to a conventional manually actuated syringe characterized in that the individual administering the medication (e.g., a health care provider or a subject who self-administers the medication) must directly depress a plunger into a barrel containing medication in order to effect the injection. It will be appreciated that a pump may be powered electrically or mechanically, e.g., as described herein. In some aspects, a delivery device comprising a pump may allow for convenient administration of doses according to a dosing regimen described herein.

In certain embodiments, the delivery device is portable. A portable device, also referred to as an “ambulatory” device, can be sufficiently light in weight and have appropriate dimensions so as to permit the subject to move about freely while the device is in use. In certain embodiments, such device does not require attachment to a pole or power outlet. In some embodiments a portable delivery device may be attached to a belt or shoulder strap or worn in a case that may be attached to a belt or shoulder strap, or may be placed in a pocket of a garment.

One of ordinary skill in the art appreciates that a pump may operate in any of a variety of ways and may utilize a variety of energy sources, e.g., disposable or rechargeable batteries, alternating current power supply (e.g., via a wall socket in a building), compressed gas, or energy stored in a compressed spring or in a stretched expandable resilient chamber. A device in which fluid is held in a stretchable balloon reservoir, and pressure from the elastic walls of the balloon reservoir drives fluid delivery may be referred to as an “elastomeric infusion pump”.

In some embodiments, a delivery device comprises a pump and a syringe containing a liquid to be administered and removably associated with the device, and a driving unit, which may be electronically controlled by a controller, arranged to make the plunger of the syringe slide so as to cause infusion of the liquid directly or via flexible tubing through a piercing member such as a needle or cannula that is introduced into the subject's body under the skin. For example, in some embodiments a pump may comprise a motor that turns a screw that pushes the plunger on a syringe that contains the liquid. Pushing of the plunger causes liquid to be expelled from the syringe and introduced into the subject's body via an attached piercing member. Exemplary pumps are described in, e.g., U.S. Pat. Nos. 6,447,487; 6,592,551; 6,645,177; 8,187,228; US Patent Application Publication Nos. 20020123740, 20030229311, 20060184123, 20070100281, 20090123309, 20150038906. The Crono PID (NDC No.: 8423.2000.02), Crono S-PID30, and Crono S-PID 50 (NDC No.: 8423.2000.04) (Canè s.r.l. Medical Technology (Rivoli, Italy)), and the T34™ Ambulatory Syringe Pump and the T60™ Ambulatory Syringe Pump (CME Medical, Blackpool, UK) are exemplary portable syringe infusion pumps that may be used in certain embodiments.

In some embodiments the pump may be electronically programmable or controlled. In some embodiments the pump is not electronically programmable or controlled.

In some embodiments a pump uses electricity as a source of power. In some embodiments a pump does not use electricity as a source of power. Such a pump may, for example, use a compressed spring or compressed gas as an energy source.

In some embodiments the pump is a constant-pressure pump that applies a constant pressure to depress the barrel of a syringe containing the liquid to be administered. An example of a constant-pressure pump is the Freedom60® infusion system (RMS Medical Products, Chester, NY). In some embodiments a FreedomEdge® infusion system (RMS Medical Products) may be used, e.g., with a syringe capable of holding up to 20 ml or a syringe capable of holding up to 30 ml. Another example of a constant pressure device is the SCIg60 syringe pump (EMED Technologies, El Dorado Hills, CA). In some embodiments a valve may control the flow rate of the liquid. In some embodiments tubing connected to the syringe may control the flow rate of the liquid, e.g., as described in U.S. patents application Nos. 20150374911 and/or 20160256625. In some embodiments a delivery rate of between 0.5 ml/minute and 1 ml/minute may be used.

In some embodiments the liquid to be administered is contained in a pressurized chamber prior to administration. In some embodiments the liquid is contained in a resilient, expandable container portion such as a bladder or balloon prior to delivery. The expandable container portion may be made of or comprise an inner lining of compatible medical grade butyl, silicone or other material suitable for holding the liquid. The container portion expands upon filling with liquid (e.g., with a unit dose of the compound to be administered), so as to exert pressure on the liquid. One of ordinary skill in the art appreciates that the container portion may be filled in a variety of ways. In some embodiments filling of the expandable container portion may be accomplished manually, e.g., using a manually actuated syringe, or may be performed using a filling apparatus. After the device is attached to the subject's skin, a piercing member such as a needle or cannula, which may be spring loaded, may automatically or following additional activation, such as by pressing a button, emerge from the device's housing and pierce the skin. Subsequently, either automatically or following additional activation, such as by pressing a button, pressure forces the liquid out of the chamber or container and into the subject's body via the needle or cannula. Exemplary devices are described in US Patent Application Pub. Nos. 20130018326, and/or 20150217058.

In some embodiments the delivery device is an “on-body delivery device”, which term refers to a delivery device comprising a chamber or other container portion for holding a liquid to be administered to a subject, wherein the device can deliver the liquid while attached directly to the subject's skin without the need for a separate support or external reservoir and, typically, permits the subject to be mobile during delivery. The chamber for holding the liquid may be contained in a housing. Typically, an on-body delivery device is affixed to the subject's skin using an adhesive. The device is affixed sufficiently strongly so that the device is self-supporting. The device may be provided with an adhesive layer, e.g., on the outer surface of the housing, for use to secure the device directly to the skin. The adhesive layer may surround the portion of the device from which a piercing member such as a needle or cannula projects so as to provide a seal around the penetrated skin. In some embodiments an on-body delivery device is available from Sensile Medical AG (Hagendorf, Switzerland). For example, devices known as SenseInfuse, SensePatch, or Senseflex, may be used. In some embodiments an on-body delivery device is available from Enable Injections, Inc. (Cincinnati, OH). In some embodiments the device that comprises a resilient, expandable container portion such as a bladder or balloon to expel the liquid is an on-body delivery device. In some embodiments the device, e.g., an on-body delivery device, is configured such that the piercing member, e.g., needle, is not visible to the user prior to or during use of the device. In some embodiments, the piercing member, e.g., needle, may retract when delivery of the liquid is complete or when the device is removed from the skin. It will be appreciated that a piercing member, e.g., a needle, for use with a delivery device described herein may have any suitable gauge or inner diameter, e.g., such gauge or inner diameters as described elsewhere herein.

In some embodiments, a delivery device comprises a housing into which a vial, cartridge, or syringe containing a liquid (e.g., a liquid comprising a LACA) may be inserted. The liquid is administered upon activation of the device. In some embodiments the liquid is transferred to a chamber of the device prior to administration. In some embodiments a delivery device is reusable, e.g., it can be re-filled or supplied with a new vial, cartridge, or syringe following administration of the contents.

In some embodiments a delivery device is a single use device, i.e., the device is designed to be used to administer a single dose or for use in a single administration session. For example, a device may be designed to be affixed to the skin of a subject, activated to administer a dose, removed, and then recycled or discarded rather than used to administer one or more additional doses.

In some embodiments a delivery device that allows delivery of a liquid into two or more sites may be used. In some embodiments the number of sites is between 1 and 5. In some embodiments the number of sites is greater than 5, e.g., between 6 and 10. Delivery to the two or more sites may be simultaneous or sequential. The device may comprise a pair of syringes, each arranged to be connected to one of the sites and coupled to a body that houses a driving system of the device. Exemplary devices are described in WO2011154928 and US Patent Application Publication No. 20120143133. In some embodiments a multi-needle infusion set may be used. In some embodiments a multi-needle infusion set comprises a flexible tube that communicates at one end with a chamber (which term is used interchangeably with “reservoir”) containing the liquid (e.g., a syringe) while the other end bifurcates into multiple tubes each having a needle at the end. The Neria™ multi infusion sets (Unomedical A/S, Osted, Denmark) are exemplary multi-needle infusion sets.

In some embodiments a delivery device may collect data regarding use of the device. Such data may comprise, for example, the date and time at which the device was used, delivery parameters such as the volume administered, the duration of administration, whether any problems occurred during administration, etc. The data may be stored on a computer-readable medium physically associated with the device and/or may be transmitted to a remote location, e.g., a remote server, where it may be stored, analyzed, or further transmitted for storage or analysis. The device may comprise one or more processors, sensors, software programs, and appropriate connectivity that allow data to be exchanged between the device and other products and systems. Data may be transferred via radio-frequency identification (RFID), bar-code/QR-code scanning, cellular, Bluetooth low energy (BTLE), physical wire, or a combination thereof. The data may be transmitted over any suitable network, e.g., the Internet. The data may be analyzed and/or stored in the Cloud. In some embodiments the device comprises an active or passive RFID tag or chip, hereinafter referred to as an “RFID tag”. The RFID tag may contain data that identifies the device. The RFID tag may be an active tag or chip that signals usage-related information such as activation of the device and/or completion of an administration of a dose. In some embodiments data acquired from a particular device may be made available to one or more entities or individuals, such as health care providers or caregivers of the subject. Such entities or individuals may additionally or alternately be automatically notified of the occurrence or non-occurrence of specified events. For example, if a dose is not administered on a day on which such administration is to take place according to the dosing schedule, or if the device is deployed on a day when administration is not supposed to take place according to the dosing schedule, one or more health care providers or caregivers of the subject may be notified. Once notified, an entity or individual may take appropriate action, such as contacting the subject. In some embodiments a monitoring system automatically attempts to contact the subject, e.g., by phone or text message, if a dose is not administered as scheduled.

In some embodiments a delivery system may comprise a delivery device and a remote control device. The remote control device may, for example, allow programming of the delivery device and/or may be used to activate the delivery device to start delivery of the fluid or to cause the delivery device to cease delivery of the fluid.

In some embodiments, the present disclosure contemplates providing to a subject (e.g., by mail or arranged pickup or other regular mode of delivery) a set of devices as described herein that together provide a supply of active agent (e.g., LACA) sufficient to last for a predetermined period of time (e.g., one week, two weeks, three weeks, four weeks, etc.). In some embodiments, such a set is sent to the patient's residence on a regular basis (e.g., every week, two weeks, three weeks, four weeks, etc.) with a timing selected such that the patient does not run out. In some embodiments, a composition (e.g., comprising a LACA) may be contained in a container (e.g., a vial) or in any of the herein-mentioned drug delivery devices or packs. In some embodiments the supply is sufficient to last for between 4 and 12 weeks, between 12 and 26 weeks, or more.

Those skilled in the art, reading the present disclosure, will appreciate that, in accordance with standard practice in the field, a container containing a particular volume, as described herein may include an additional volume sufficient to permit the designated particular volume (e.g., unit dose) to be withdrawn from the container for administration.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

The disclosure is further illustrated by the following examples. The examples are provided for illustrative purposes only. They are not to be construed as limiting the scope or content of the disclosure in any way.

### VI. Manufacturing

Various technologies, e.g., synthetic chemistry technologies, formulation technologies, assays, etc. can be utilized to prepare, characterization, and assess compounds, compositions and methods described herein in accordance with the present disclosure. Certain technologies are described below and in the Examples.

In some embodiments, the present disclosure provides a method for preparing a compound of formula I or a salt thereof, comprising removing one or more protection groups from a compound of formula M-I:

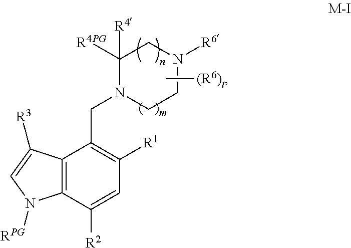

or a salt thereof to provide a compound of formula I or a salt thereof, wherein:
R^{PG} is an amino protecting group,
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group; and
each other variable is independently as described herein (e.g., see, for a compound of formula I or a pharmaceutically acceptable salt thereof).

In some embodiments, R^{PG} is —C(O)R, wherein R is optionally substituted C₁₋₆ aliphatic. In some embodiments, R is optionally substituted C₁-C₆ alkyl. In some embodiments, R is t-butyl.

In some embodiments, R^{4PG} is optionally substituted phenyl. In some embodiments, it is optionally substituted naphthyl. In some embodiments, it is optionally substituted 1,2,3,4-tetrahydronaphthalenyl. In some embodiments, it is optionally substituted 5-10 membered heteroaryl. In some embodiments, it is optionally substituted 5-6 membered heteroaryl. In some embodiments, it is optionally substituted 9-10 membered heteroaryl. In some embodiments, it is substituted with R^{5PG}. In some embodiments, it is substituted with R⁵′. In some embodiments, it is substituted with R^{5PG} but no R⁵′. In some embodiments, it is substituted with R^{5PG} and R⁵′. In some embodiments, it is substituted with R^{5PG} and two R⁵′.

In some embodiments, R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxyl, and cyanomethyl.

In some embodiments, R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R^{5PG} and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R^{4PG} is phenyl substituted with R^{5PG} and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R^{4PG} is

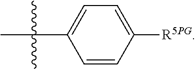

In some embodiments, R^{5PG} is protected carboxyl. In some embodiments, R^{5PG} is —C(O)R^{8PG}, wherein R^{8PG} is optionally substituted C₁₋₆ aliphatic. In some embodiments, R^{8PG} is optionally substituted C₁-C₆ alkyl. In some embodiments, R^{8PG} is methyl. In some embodiments, R^{8PG} is t-butyl.

In some embodiments, R^{6′} is R^{6′} is hydrogen. In some embodiments, R^{6′} is a primary, secondary or tertiary group. In some embodiments, at least hydrogen is bonded to the carbon in R^{6′} through which R^{6′} is bonded to the rest of a molecule. In some embodiments, R^{6′} is C₁-C₆ aliphatic, R¹⁰, —CH₂—C₃-C₆ cycloaliphatic, —CH₂—R¹⁰, —CH₂-(hydroxy C₁-C₄ alkyl), —CH₂-phenyl, —CH₂-(amino C₁-C₄ alkyl), or —CH₂-(mono- and di-C₁-C₄ alkylamino C₁-C₄ alkyl), each of which is independently optionally substituted as described herein. In some embodiments, R¹⁰ is heterocyclyl optionally substituted as described herein. In some embodiments, a method comprises a step for preparing a compound of formula M-I or a salt thereof, which step comprises a reductive amination reaction comprising contacting an aldehyde or ketone with a compound of formula M-II or a salt thereof in the presence of a reducing agent (e.g., triacetoxyborohydride):

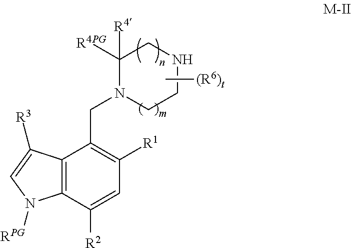

In some embodiments, an aldehyde or ketone is of such a structure that when its —C(O)— group is replaced with —CH₂—, a compound of H—R⁶ is formed. Various reductive amination technologies are available in the art and can be utilized as described herein.

Various reduction technologies may be utilized in accordance with the present disclosure. In some embodiments, R^{6′} is —C(O)—C₁-C₆ aliphatic optionally substituted as described herein. In some embodiments, a method comprises a step for preparing a compound of formula M-I or a salt thereof, which step comprises an amidation reaction comprising contacting an acylating agent with a compound of formula M-II or a salt thereof. In some embodiments, an acylating agent has the structure of R^{6x1}—C(O)—C₁-C₆ aliphatic or a salt thereof, wherein R^{6x1} is —OH or R^{6x1}—C(O)— is an activated carboxyl group. In some embodiments, R^{6x1} is —Cl. In some embodiments, R^{6x1}—C(O)—C₁-C₆ aliphatic is an anhydride. Various technologies for amidation reactions and acylating agents are available in the art and can be utilized in accordance with the present disclosure.

In some embodiments, R^{6′} is —SO₂—C₁-C₆ aliphatic optionally substituted as described herein. In some embodiments, a method comprises a step for preparing a compound of formula M-I or a salt thereof, which step comprises a sulfonylation reaction comprising contacting a sulfonylation agent with a compound of formula M-II or a salt thereof. In some embodiments, a sulfonylation agent has the structure of R^{6x2}—S(O)₂—C₁-C₆ aliphatic or a salt thereof, wherein R^{6x2}—S(O)₂— is an activated sulfonyl group. In some embodiments, R^{6x2} is —Cl. In some embodiments, a sulfonylation reagent is MsCl. Various technologies for sulfonylation reactions and sulfonylation agents are available in the art and can be utilized in accordance with the present disclosure.

In some embodiments, R^{6′} is installed through an amination reaction. In some embodiments, a method comprises a step for preparing a compound of formula M-I or a salt thereof, which step comprises an amination reaction comprising contacting a sulfonylation agent with a compound of formula M-II or a salt thereof. In some embodiments, a compound of formula M-II or a salt thereof is reacted with a compound having the structure of R^{6x3}—R⁶′, wherein R^{6x3} is a leaving group, and R^{6′} is as described herein. In some embodiments, R^{6x3} is halogen. In some embodiments, it is —Cl. In some embodiments, it is —Br. In some embodiments, it is —I. In some embodiments, R^{6′} is C₁-C₆ aliphatic as described herein. In some embodiments, R^{6′} is phenyl optionally substituted as described herein. In some embodiments, R^{6′} is phenyl. In some embodiments, R⁶ is heteroaryl optionally substituted as described herein. In some embodiments, R^{6x3}—R^{6′} is bromobenzene. In some embodiments, a reaction is performed in the presence of a base (e.g., NaOtBu, Et₃N, K₂CO₃, etc.). In some embodiments, a reaction is performed in the presence of a metal reagent, e.g., a catalyst and optionally a ligand (e.g., Pd₂(dba)₃ and RuPhos). In some embodiments, R^{6x3}—R⁶ is 1-fluoro-3-iodopropane. In some embodiments, R^{6x3}—R^{6′} is allylbromide. In some embodiments, R^{6x3}—R^{6′} is propargyl bromide. In some embodiments, R^{6x3}—R^{6′} is 2-bromoethanol. In some embodiments, R^{6x3}—R^{6′} is 1-fluoro-2-iodoethane. In some embodiments, R^{6x3}—R^{6′} is 2,2-difluoroethyl trifluoromethanesulphonate. In some embodiments, R^{6x3}—R^{6′} is 2-bromoethylmethyl ether.

Various technologies for amination reactions are available in the art and can be utilized in accordance with the present disclosure. Certain examples are described in the Examples. In some embodiments, amination is performed in the presence of a catalyst system (e.g., Pd₂(dba)₃ and RuPhos) and/or a base (e.g., NaOtBu).

Various technologies for sulfonylation reactions and sulfonylation agents are available in the art and can be utilized in accordance with the present disclosure.

In some embodiments, as described herein, t is 0. In some embodiments, m is 1. In some embodiments, n is 1. In some embodiments, R^{4′} is —H. In some embodiments, R¹ is —O—C₁-C₆ alkyl, e.g., —OMe. In some embodiments, R¹ is —H. In some embodiments, R² is —C₁-C₆ alkyl, e.g. —CH₃. In some embodiments, R² is —H. In some embodiments, R³ is —H. In some embodiments, R^{PG} is -Boc.

In some embodiments, a method comprises a step for preparing a compound of formula M-II or a salt thereof, which step comprises de-protecting a compound having the structure of M-III or a salt thereof:

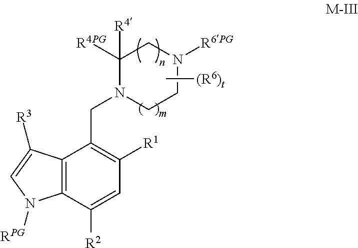

wherein R^{6′PG} is R^{6′} or an amino protecting group. In some embodiments, R^{6′PG} is an amino protecting group. In some embodiments, it can be selectively de-protected in the presence of R^{4PG} and R^{PG}. For example, as utilized in the Examples, in some embodiments, R^{6′PG} is -Bn. In some embodiments, R^{PG} is -Boc.

In some embodiments, a method comprises reacting a compound of formula M-IV or a salt thereof:

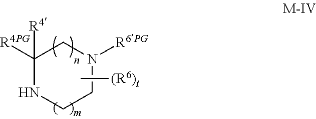

wherein each variable is independently as described herein,
with a compound of formula M-V or a salt thereof:

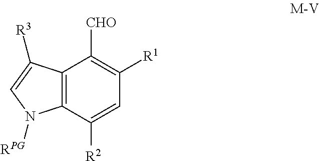

wherein each variable is independently as described herein,
under a condition to provide a compound of formula M-III or a salt thereof. In some embodiments, a condition is a reductive amination condition. Various reductive amination conditions can be utilized in accordance with the present disclosure.

In some embodiments, the present disclosure provides a method comprising protecting a group and/or de-protecting a group of a compound of formula M-VI or a salt thereof:

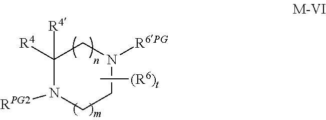

wherein R^{PG2} is an amino protecting group, and each other variable is independently as described herein, to provide a compound of formula M-IV or a salt thereof.

In some embodiments, R⁴ is

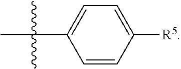

In some embodiments, R⁵ is —COOH. In some embodiments, R^{4PG} is

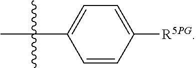

In some embodiments, R^{5PG} is —(O)—R^{8PG}. In some embodiments, R^{5PG} is —COOMe. In some embodiments, R^{PG2} and R^{6′PG} can be selectively removed in the presence of the other. In some embodiments, R^{PG2} is Boc. In some embodiments, R^{6′PG} is Bn. In some embodiments, a compound of formula M-IV is

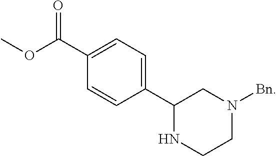 [SMILES: COC(=O)c1ccc(C2CN(Cc3ccccc3)CCN2)cc1]

In some embodiments, a salt of a compound of formula M-IV is

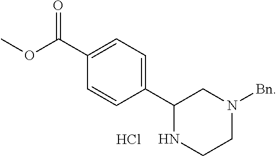 [SMILES: COC(=O)c1ccc(C2CN(Cc3ccccc3)CCN2)cc1 ; Cl]

In some embodiments, as described herein, R^{PG} is Boc. In some embodiments, R¹ is —O—C₁-C₆ alkyl. In some embodiments, R¹ is —OCH₃. In some embodiments, R² is —CH₃. In some embodiments, R³ is —H. In some embodiments, a compound of formula M-V is

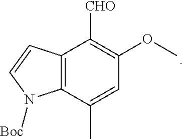 [SMILES: COc1cc(C)c2c(ccn2C(=O)OC(C)(C)C)c1C=O]

In some embodiments, a compound of formula M-VI is

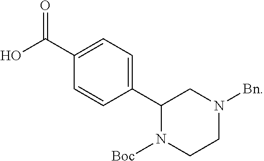 [SMILES: CC(C)(C)OC(=O)N1CCN(Cc2ccccc2)CC1c1ccc(C(=O)O)cc1]

In some embodiments, the present disclosure provides a method comprising converting a compound of formula M-VII or a salt thereof:

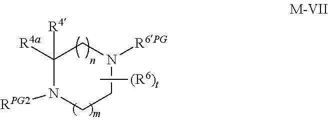

wherein:
R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5a} and further optionally substituted with one or two R⁵′;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom; and
   each other variable is independently as described herein, to provide a compound of formula M-VI or a salt thereof.

In some embodiments, R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5a} and further optionally substituted with one or two R⁵′. In some embodiments, it is optionally substituted naphthyl. In some embodiments, it is optionally substituted 1,2,3,4-tetrahydronaphthalenyl. In some embodiments, it is optionally substituted 5-10 membered heteroaryl. In some embodiments, it is optionally substituted 5-6 membered heteroaryl. In some embodiments, it is optionally substituted 9-10 membered heteroaryl. In some embodiments, it is substituted with R^{5a}. In some embodiments, it is substituted with R⁵′. In some embodiments, it is substituted with R^{5a} but no R⁵′. In some embodiments, it is substituted with R^{5a} and R⁵′. In some embodiments, it is substituted with R^{5a} and two R⁵′.

In some embodiments, R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5a} and further substituted with 0 or 1 substituent selected from halogen, C₁-C₄ alkyl, C₁-C₄ alkoxy, hydroxy C₁-C₄ alkyl, hydroxyl, and cyanomethyl. In some embodiments, R^{4a} is substituted with —CHO. In some embodiments, R⁴ is substituted with —COOH. In some embodiments, R^{4a} is

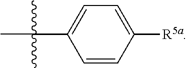

In some embodiments, R^{5a} is —CHO. In some embodiments, R⁴ is

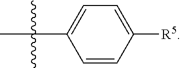

In some embodiments, R⁵ is —COOH. In some embodiments, a reaction is an oxidation reaction which converts an aldehyde into a carboxylic acid group. In some embodiments, t is 0. In some embodiments, R^{PG2} is Boc. In some embodiments, R^{6′PG} is Bn. In some embodiments, a compound of formula M-VII is

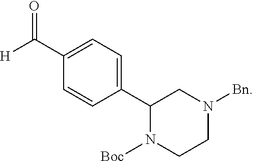 [SMILES: CC(C)(C)OC(=O)N1CCN(Cc2ccccc2)CC1c1ccc(C=O)cc1]

Various such oxidation technologies are available and can be utilized in accordance with the present disclosure.

In some embodiments, a method comprising protecting an amino group, e.g., in a compound of formula M-VIII or a salt thereof:

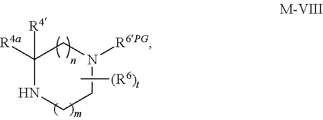

wherein each variable is independently as described herein, to provide a compound of formula M-VII or a salt thereof. In some embodiments, R^{RPG2} is Boc. In some embodiments, R^{6′PG} is Boc. In some embodiments, R^{4a} is substituted with —CHO. In some embodiments, R^{4a} is

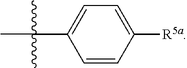

In some embodiments, R^{5a} is —CHO. In some embodiments, t is 0. In some embodiments, a compound of formula M-VIII is

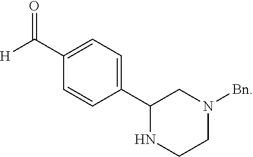 [SMILES: O=Cc1ccc(C2CN(Cc3ccccc3)CCN2)cc1]

In some embodiments, the present disclosure provides a method comprising converting a compound of formula M-IX or a salt thereof:

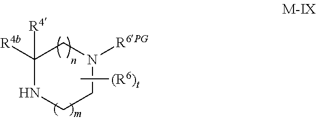

wherein:
R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5b} and further optionally substituted with one or two R⁵′;
R^{5b} is —CH₂OH, —CH₂ CH₂OH, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom; and
   each other variable is independently as described herein, to provide a compound of formula M-VIII or a salt thereof.

In some embodiments, R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5b} and further optionally substituted with one or two R⁵′. In some embodiments, it is optionally substituted naphthyl. In some embodiments, it is optionally substituted 1,2,3,4-tetrahydronaphthalenyl. In some embodiments, it is optionally substituted 5-10 membered heteroaryl. In some embodiments, it is optionally substituted 5-6 membered heteroaryl. In some embodiments, it is optionally substituted 9-10 membered heteroaryl. In some embodiments, it is substituted with R^{5b}. In some embodiments, it is substituted with R⁵′. In some embodiments, it is substituted with R^{5b} but no R⁵′. In some embodiments, it is substituted with R^{5b} and R⁵′. In some embodiments, it is substituted with R^{5b} and two R⁵′.

In some embodiments, R^{4b} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5b} and further substituted with 0 or 1 substituent selected from halogen, C₁-C₄ alkyl, C₁-C₄ alkoxy, hydroxy C₁-C₄ alkyl, hydroxyl, and cyanomethyl.

In some embodiments, R^{4b} is substituted with —CH₂OH. In some embodiments, R^{4a} is substituted with —CHO. In some embodiments, R^{4a} is

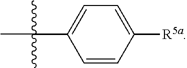

In some embodiments, R^{5a} is —CHO. In some embodiments, R^{4b} is

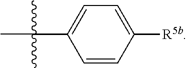

In some embodiments, R^{5b} is —CH₂OH. In some embodiments, a reaction is an oxidation reaction which converts a primary alcohol into an aldehyde. In some embodiments, t is 0. In some embodiments, a compound of formula M-IX is

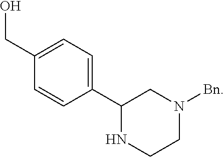 [SMILES: OCc1ccc(C2CN(Cc3ccccc3)CCN2)cc1]

Various oxidation technologies are available and can be utilized in accordance with the present disclosure.

In some embodiments, the present disclosure provides a method comprising reducing a compound of formula M-X or a salt thereof:

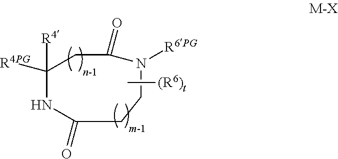

wherein each variable is independently as described herein, to provide a compound of formula M-IX or a salt thereof. In some embodiments, R^{4PG} is

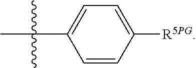

In some embodiments, R^{5PG} is —(O)—R^{8PG}. In some embodiments, R^{5PG} is —COOMe. In some embodiments, n is 1. In some embodiments, m is 1. In some embodiments, R^{6′PG} is -Bn. In some embodiments, a carboxyl or a derivative thereof (e.g., an ester) is reduced to a hydroxyl. In some embodiments, t is 0. In some embodiments, a compound of formula M-X is

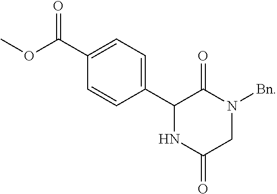 [SMILES: COC(=O)c1ccc(C2NC(=O)CN(Cc3ccccc3)C2=O)cc1]

Various reduction technologies are available and can be utilized in accordance with the present disclosure.

In some embodiments, the present disclosure provides a method comprising converting a compound of formula M-XI or a salt thereof:

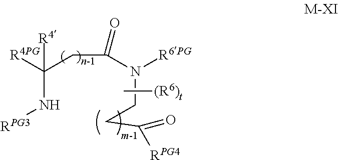

wherein:
R^{PG3} is —H or an amino protecting group;
R^{PG4} is —OH or —C(O)R^{PG4} is a protected carboxyl group; and
each variable is independently as described herein, to provide a compound of formula M-X or a salt thereof.

In some embodiments, R^{PG3} is hydrogen. In some embodiments, it is an amino protecting group. In some embodiments, it is -Boc. In some embodiments, R^{PG4} is —OH. In some embodiments, it is a carboxy protecting group. In some embodiments, it is —O-optionally substituted C₁-C₆ aliphatic. In some embodiments, it is —O-optionally substituted C₁-C₆ alkyl. In some embodiments, it is —OMe. In some embodiments, t is 0. In some embodiments, a compound of formula M-XI is

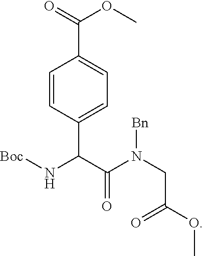 [SMILES: COC(=O)CN(Cc1ccccc1)C(=O)C(NC(=O)OC(C)(C)C)c1ccc(C(=O)OC)cc1]

In some embodiments, the present disclosure provides a method comprising reacting a compound of formula M-XII or a salt thereof:

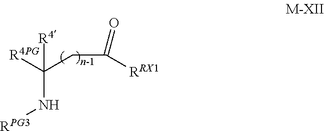

wherein R^{RX1} is —OH or —C(O)R^{RX1} is an activated carboxyl group, and each other variable is independently as described herein, with a compound of formula M-XIII or a salt thereof:

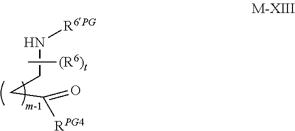

wherein each variable is independently as described herein, to provide a compound of formula M-XI or a salt thereof.

In some embodiments, R^{PG3} is Boc. In some embodiments, R^{RX1} is —OH. In some embodiments, a compound of formula M-XII is

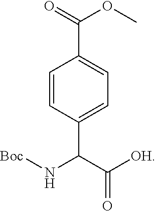 [SMILES: COC(=O)c1ccc(C(NC(=O)OC(C)(C)C)C(=O)O)cc1]

In some embodiments, R^{6′PG} is -Bn. In some embodiments, t is 0. In some embodiments, R^{PG4} is optionally substituted —O—C₁-C₆ aliphatic. In some embodiments, it optionally substituted —O—C₁-C₆ alkyl. In some embodiments, it is —OCH₃. In some embodiments, a compound of formula M-XIII is

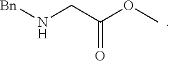

In some embodiments, the present disclosure provides a method, comprising converting a compound of formula M-XIV or a salt thereof:

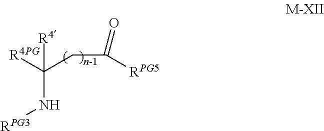

wherein:
—C(O)R^{PG5} is protected carboxyl, and each other variable is independently as described herein, to a compound of formula M-XII or a salt thereof.

In some embodiments, —C(O)R^{PG5} is —C(O)—O-optionally substituted C₁-C₆ aliphatic. In some embodiments, it is —O-optionally substituted C₁-C₆ alkyl. In some embodiments, it is —OMe. In some embodiments, —C(O)R^{PG5} can be selectively deprotected in the presence of R^{PG3}. In some embodiments, R^{PG3} is Boc. In some embodiments, n is 0. In some embodiments, a compound of formula M-XIV is

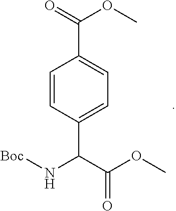 [SMILES: COC(=O)c1ccc(C(NC(=O)OC(C)(C)C)C(=O)OC)cc1]

In some embodiments, a method comprises reacting a compound of formula M-IV′ or a salt thereof:

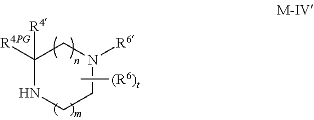

wherein each variable is independently as described herein,
with a compound of formula M-V or a salt thereof:

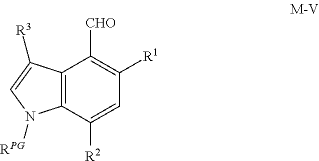

wherein each variable is independently as described herein,
under a condition to provide a compound of formula M-I or a salt thereof. In some embodiments, a condition is a reductive amination condition. Various reductive amination conditions can be utilized in accordance with the present disclosure.

In some embodiments, R^{6′} is cyclopropyl. In some embodiments, t is 0. In some embodiments, m is 1. In some embodiments, n is 1. In some embodiments, R^{4′} is hydrogen. In some embodiments, a compound of formula M-IV′ is

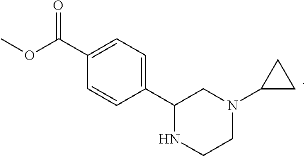 [SMILES: COC(=O)c1ccc(C2CN(C3CC3)CCN2)cc1]

In some embodiments, a salt of a compound of formula M-IV′ is

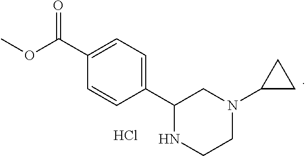 [SMILES: COC(=O)c1ccc(C2CN(C3CC3)CCN2)cc1 ; Cl]

In some embodiments, a compound of formula M-V is

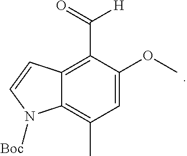 [SMILES: COc1cc(C)c2c(ccn2C(=O)OC(C)(C)C)c1C=O]

In some embodiments, a compound of formula M-I is

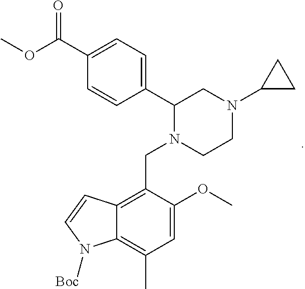 [SMILES: COC(=O)c1ccc(C2CN(C3CC3)CCN2Cc2c(OC)cc(C)c3c2ccn3C(=O)OC(C)(C)C)cc1]

In some embodiments, the present disclosure provides a method comprising de-protecting a group of a compound of formula M-XV or a salt thereof:

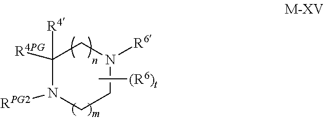

wherein each variable is independently as described herein, to provide a compound of formula M-IV′ or a salt thereof. In some embodiments, R^{PG2} is Boc. In some embodiments, a compound of formula M-XV is

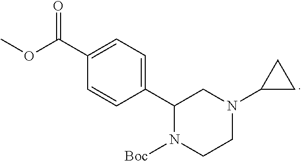 [SMILES: COC(=O)c1ccc(C2CN(C3CC3)CCN2C(=O)OC(C)(C)C)cc1]

In some embodiments, a method comprises a step for preparing a compound of formula M-XV or a salt thereof, which step comprises a reductive amination reaction comprising contacting an aldehyde or ketone with a compound of formula M-XVI or a salt thereof in the presence of a reducing agent (e.g., triacetoxyborohydride):

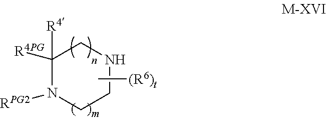

In some embodiments, a method comprises a step for preparing a compound of formula M-XV or a salt thereof, which step comprises amination reaction comprising contacting a suitable reagent with a compound of formula M-XVI or a salt thereof to provide a compound of formula M-XV or a salt thereof. In some embodiments, a method comprises a step for preparing a compound of formula M-XV or a salt thereof, which step comprises amidation or sulfonylation reaction comprising contacting a suitable reagent with a compound of formula M-XVI or a salt thereof to provide a compound of formula M-XV or a salt thereof. In some embodiments, a sulfonylation agent has the structure of R^{6x2}—S(O)₂—C₁-C₆ aliphatic or a salt thereof as described herein. In some embodiments, an acylating agent has the structure of R^{6x1}—C(O)—C₁-C₆ aliphatic or a salt thereof as described herein.

Various reduction technologies may be utilized in accordance with the present disclosure. In some embodiments, a compound of formula M-XVI is

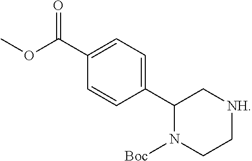 [SMILES: COC(=O)c1ccc(C2CNCCN2C(=O)OC(C)(C)C)cc1]

In some embodiments, a suitable reagent is a boronic acid reagent. In some embodiments, a suitable reagent is cyclopropylboronic acid. In some embodiments, a reaction is performed with a base (e.g. sodium carbonate) and/or a metal salt (e.g., copper (II) acetate).

In some embodiments, the present disclosure provides a method comprising de-protecting a group of a compound of formula M-XVII or a salt thereof:

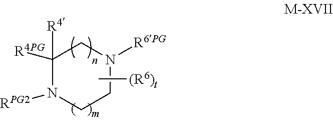

wherein each variable is independently as described herein, to provide a compound of formula M-XVI or a salt thereof. In some embodiments, R^{PG2} is Boc. In some embodiments, R^{6′PG} is -Bn. In some embodiments, a compound of formula M-XVII is

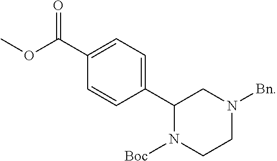 [SMILES: COC(=O)c1ccc(C2CN(Cc3ccccc3)CCN2C(=O)OC(C)(C)C)cc1]

In some embodiments, the present disclosure provides a method comprising protecting a group of a compound of formula M-IV or a salt thereof to provide a compound of formula M-XVII or a salt thereof. In some embodiments, R^{PG2} is Boc. In some embodiments, R^{6′PG} is -Bn. In some embodiments, a compound of formula M-IV is

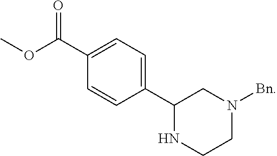 [SMILES: COC(=O)c1ccc(C2CN(Cc3ccccc3)CCN2)cc1]

In some embodiments, a salt of a compound of formula M-IV is

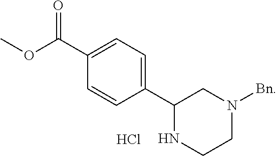 [SMILES: COC(=O)c1ccc(C2CN(Cc3ccccc3)CCN2)cc1 ; Cl]

In some embodiments, the present disclosure provides a method, comprising:
reacting a compound having the structure of formula M-XIII or a salt thereof with a compound having the structure of formula M-XII′:

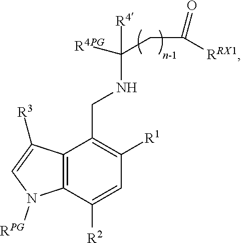

or a salt thereof to provide a compound of formula M-X′:

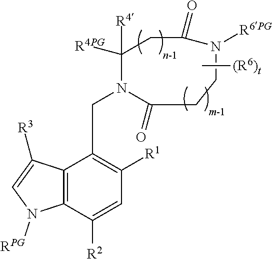

or a salt thereof.

In some embodiments, R^{6PG} is —H. In some embodiments, R^{PG4} is —OH. In some embodiments, R^{PG} is Tos. In some embodiments, a compound having the structure of M-X′ or a salt thereof is converted into a compound having the structure of formula M-X″:

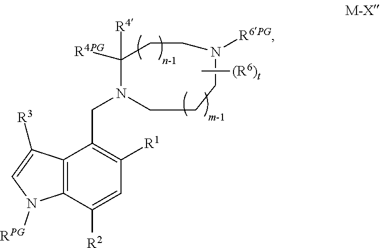

or a salt thereof. In some embodiments, R^{6′PG} is R^{6′} as described herein. In some embodiments, R^{6′PG} is R^{6′} and is taken together with one R⁶ to from an optionally substituted ring as described herein.

Certain useful technologies are described as General Experimental Procedures in the Examples.

Many compounds have been reported to be useful for modulating complement activities, complement factor B functions, etc., e.g., those reported in WO 2015/009616, WO 2019/043609, WO 2022/143845, WO 2022/028527, WO 2022/028507, etc., the compounds (specific compound, compounds represented by formulae, etc.) of each of which are incorporated herein by reference. Among other things, the present disclosure encompasses the recognition that certain structure elements disclosed herein, e.g., central ring systems comprising two or more nitrogen atoms (e.g., piperazine rings, homopiperazine rings, bicyclic or polycyclic rings, etc.), substitutions (e.g., amino groups or derivatives thereof (e.g., amides, carbamates, etc.)) on rings bonded to acidic groups (e.g., —COOH) or bioisosteres or derivatives thereof (e.g., esters, amides, etc.) (e.g., at positions next to such acidic groups or bioisosteres or derivatives thereof) can be utilized to provide biological active compounds, in some embodiments, with improved activities and/or properties (e.g., pharmacokinetic or pharmacodynamic properties, toxicity profiles, etc.). In some embodiments, structural elements are utilized to modifying reported compounds, e.g., replacing corresponding elements in such reported compounds, e.g., those reported in WO 2015/009616, WO 2019/043609, WO 2022/143845, WO 2022/028527, WO 2022/028507, etc. In some embodiments, the present disclosure provides a method, comprising utilizing a structural element described herein to replace a corresponding element in a reported compound. In some embodiments, the present disclosure provides a compound which is a product of such a method.

In various embodiments, reactions are performed in solvent systems, e.g., those exemplified in the samples. In some embodiments, a solvent system is or comprises a polar solvent. In some embodiments, a solvent system is or comprises a non-polar solvent. In some embodiments, a solvent system is or comprises a hydrocarbon solvent. In some embodiments, a solvent system is or comprises a protic solvent. In some embodiments, a solvent system is or comprises water. In some embodiments, a solvent system is anhydrous. In some embodiments, a solvent system is or comprises an alcohol.

In some embodiments, certain reactions may be performed at room temperature. In some embodiments, certain reactions are performed at about 23° C. In some embodiments, certain reactions are performed at lowered temperatures, e.g., 0° C., −10° C., −20° C., etc. In some embodiments, certain reactions are performed at increased temperatures, e.g., about 30-200, 40-150, or about 30, 40, 50, 60, 70, 80, 90, 100, 110, or 150° C. In some embodiments, temperatures may change during reactions, e.g., increasing from a lowered temperature, decreasing from an increased temperature, or combinations thereof. In some embodiments, certain reactions may be performed at increased or decreased pressure.

Many technologies are available for isolating, purifying or characterizing chemical compounds and can be utilized in accordance with the present disclosure. For example, in some embodiments, one or more of extraction, filtration, crystallization, and distillation are utilized to isolate desired products and/or remove undesired byproducts or impurities. In some embodiments, chromatography, e.g., column chromatography, GC, HPLC, SFC, etc. is utilized for separation and/or characterization.

In some embodiments, compounds comprise chiral elements, e.g., carbon chiral centers and two or more stereoisomers (e.g., enantiomers, diastereomers, etc.) may be formed. In some embodiments, stereoselective technologies are utilized so desired stereoisomers are formed. In some embodiments, separation technologies, e.g., crystallization in an asymmetric environment (e.g., with a chirally pure reagent), chiral chromatography, etc., are utilized to isolated, purify or characterize stereoisomers. In some embodiments, a stereoisomer is an enantiomer. In some embodiments, a provided compound is enriched for a particular stereoisomer. In some embodiments, a provided compound is enriched for a particular diastereomer. In some embodiments, a provided compound is enriched for a particular enantiomer. In some embodiments, an enrichment level is a stereopurity, e.g., a diastereomeric purity, an enantiomeric purity, etc. as described herein. In some embodiments, an enrichment level is about 80%-100% (e.g., about 80%, 85%, 90%, 91%, 92%, 93%, 94%, 95%, 96%, 97%, 98%, 99%, 99.5%, 99.9% or more, or about 85%-100%, 90%-100%, 91%-100%, 92%-100%, 93%-100%, 94%-100%, 95%-100%, 96%-100%, 97%-100%, 98%-100%, 99%-100%, 95-99%, 95%-99.5%, 95%-99.9%, etc) as described herein.

Those skilled in the art appreciate that in addition to the compounds specified in provided methods, other reagents and/or conditions may be utilized in accordance with the present disclosure. For example, in some de-protecting reactions, an acid or base may be utilized. In some embodiments, in oxidation reactions, oxidation reagents are utilized. In some embodiments, in reduction reactions, reduction reagents are utilized. Many suitable reagents and conditions are reported and can be utilized in accordance with the present disclosure.

Properties and activities of provided compounds and compositions may be assessed using a variety of technologies in accordance with the present disclosure. In some embodiments, modulation of C3 convertase activity is assessed. In some embodiments, inhibition of C3 convertase is assessed. In some embodiments, modulation of complement activation is assessed. In some embodiments, inhibition of complement activation is assessed. Certain useful technologies are described in the Examples.

Among other things, the present disclosure provides the following Embodiments:

1. A compound, wherein the compound has the structure of formula I:

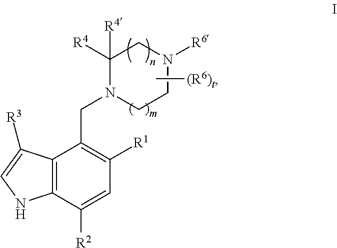

or a pharmaceutically acceptable salt thereof, wherein:
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R⁵ and further optionally substituted with one or two R^{5′;}
each R⁵′ is independently selected from halogen, C₁-C₄ alkyl, C₁-C₄ alkoxy, hydroxy C₁-C₄ alkyl, hydroxyl, R¹⁰, —OR⁶, —N(R⁵¹)₂, halo C₁-C₆ alkyl, cyano, and cyanomethyl; or
two R⁵′ are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R^{6′};
R^{4′} is hydrogen, C₁-C₄ alkyl, or hydroxy C₁-C₄ alkyl;
R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   2. A compound, wherein the compound has the structure of formula M-I:

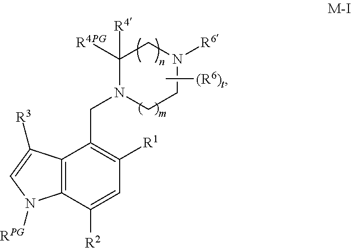

or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R^{5′;}
each R^{5′} is independently selected from halogen, C₁-C₄ alkyl, C₁-C₄ alkoxy, hydroxy C₁-C₄ alkyl, hydroxyl, R¹⁰, —OR⁶, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   3. A compound, wherein the compound has the structure of formula M-II:

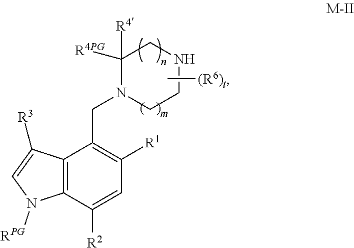

or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG}), or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   4. A compound, wherein the compound has the structure of formula M-III:

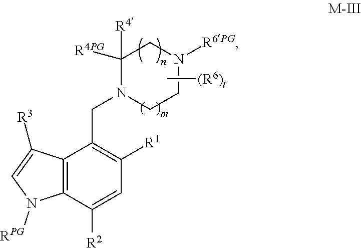

or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R^{5′;}
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   5. A compound having the structure of formula M-XII′:

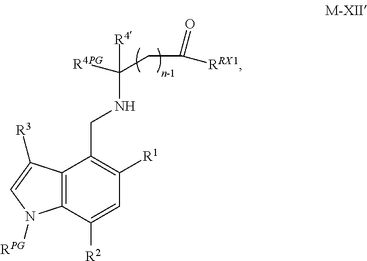

or a salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group; R^{RX1} is —OH or —C(O) R^{RX1} is an activated carboxyl group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   6. A compound, wherein the compound has the structure of formula M-X′:

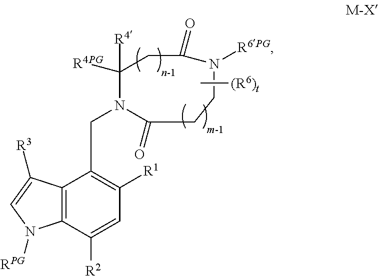

or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R^{5′;}
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   7. A compound, wherein the compound has the structure of formula M-X″:

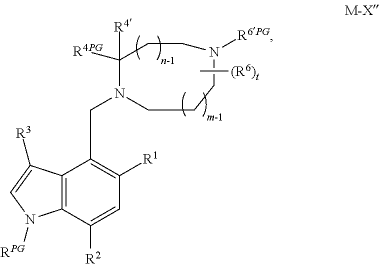

or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R^{5′;}
each R⁵ is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   8. The compound of any one of the preceding Embodiments, wherein R¹ is selected from C₁-C₆ alkoxy, C₁-C₆ alkoxy C₁-C₆ alkyl, C₁-C₆ alkoxy C₁-C₆ alkoxy, C₃-C₆ cycloalkyl C₁-C₆ alkoxy, and halo C₁-C₆ alkoxy.
   9. The compound of any one of the preceding Embodiments, wherein R¹ is C₁-C₆ alkoxy.
   10. The compound of any one of the preceding Embodiments, wherein R¹ is C₁-C₄ alkoxy.
   11. The compound of any one of the preceding Embodiments, wherein R¹ is C₁-C₂ alkoxy.
   12. The compound of any one of the preceding Embodiments, wherein R¹ is methoxy.
   13. The compound of any one of Embodiments 1-8, wherein R¹ is halo C₁-C₆ alkoxy.
   14. The compound of any one of Embodiments 1-8 and 13, wherein R¹ is halo C₁-C₄ alkoxy.
   15. The compound of any one of Embodiments 1-8 and 14, wherein R¹ is halo C₁-C₂ alkoxy.
   16. The compound of any one of Embodiments 1-8 and 16, wherein R¹ is halo C₁-C₂ alkoxy.
   17. The compound of any one of Embodiments 1-8 and 16, wherein R¹ is —OCHF₂.
   18. The compound of any one of Embodiments 1-4, wherein R¹ is C₁-C₆ alkyl.
   19. The compound of Embodiment 16, wherein R¹ is C₁-C₄ alkyl.
   20. The compound of Embodiment 17, wherein R¹ is C₁-C₂ alkyl.
   21. The compound of any one of Embodiments 1-4, wherein R¹ is C₃-C₆ cycloalkyl.
   22. The compound of any one of Embodiments 1-4, wherein R¹ is C₃-C₅ cycloalkyl.
   23. The compound of Embodiment 19, wherein R¹ is cyclopropyl.
   24. The compound of any one of the preceding Embodiments, wherein R² is selected from C₁-C₆ alkyl and halogen.
   25. The compound of any one of the preceding Embodiments, wherein R² is C₁-C₆ alkyl.
   26. The compound of any one of the preceding Embodiments, wherein R² is C₁-C₄ alkyl.
   27 The compound of any one of the preceding Embodiments, wherein R² is C₁-C₂ alkyl.
   28. The compound of any one of the preceding Embodiments, wherein R² is methyl.
   29. The compound of any one of Embodiments 1-24, wherein R² is halogen.
   30. The compound of any one of Embodiments 1-24 and 29, wherein R² is selected from chloro and fluoro.
   31. The compound of Embodiment 26, wherein R² is chloro.
   32. The compound of Embodiment 26, wherein R² is fluoro.
   33. The compound of any one of Embodiments 1-24 and 29, wherein R² is selected from bromo and iodo.
   34. The compound of Embodiment 29, wherein R² is bromo.
   35. The compound of Embodiment 29, wherein R² is iodo.
   36. The compound of any one of Embodiments 1-23, wherein R² is selected from hydrogen, C₁-C₆ alkoxy, and hydroxy C₁-C₆ alkyl.
   37. The compound of any one of the preceding Embodiments, wherein R² is hydrogen.
   38. The compound of any one of Embodiments 1-23 and 36, wherein R² is C₁-C₆ alkoxy.
   39. The compound of any one of Embodiments 1-23 and 36, wherein R² is C₁-C₄ alkoxy.
   40. The compound of any one of Embodiments 1-23 and 36, wherein R² is methoxy.
   41. The compound of any one of Embodiments 1-23 and 36, wherein R² is hydroxy C₁-C₆ alkyl.
   42. The compound of any one of Embodiments 1-23 and 36, wherein R² is hydroxy C₁-C₄ alkyl.
   43. The compound of any one of the preceding Embodiments, wherein R³ is selected from hydrogen, halogen, and cyano.
   44. The compound of any one of the preceding Embodiments, wherein R³ is hydrogen.
   45. The compound of any one of Embodiments 1-43, wherein R³ is halogen.
   46. The compound of Embodiment 45, wherein R³ is selected from fluoro and chloro.
   47. The compound of Embodiment 46, wherein R³ is fluoro.
   48. The compound of Embodiment 46, wherein R³ is chloro.
   49. The compound of Embodiment 45, wherein R³ is selected from bromo and iodo.
   50. The compound of Embodiment 49, wherein R³ is bromo.
   51. The compound of Embodiment 49, wherein R³ is iodo.
   52. The compound of any one of Embodiments 1-43, wherein R³ is selected from C₁-C₄ alkyl, halo C₁-C₄ alkyl, and —CH₂C(O)R⁷.
   53. The compound of Embodiment 52, wherein R³ is C₁-C₆ alkyl.
   54. The compound of Embodiment 52, wherein R³ is C₁-C₄ alkyl.
   55. The compound of Embodiment 54, wherein R³ is methyl.
   56. The compound of Embodiment 52, wherein R³ is halo C₁-C₄ alkyl.
   57. The compound of Embodiment 56, wherein R³ is halo C₁-C₂ alkyl.
   58. The compound of Embodiment 52, wherein R³ is —CH₂C(O)R⁷.
   59. A compound, wherein the compound has the structure of formula M-IV:

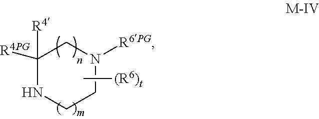

or a pharmaceutically acceptable salt thereof, wherein:
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   60. A compound, wherein the compound has the structure of formula M-IV′:

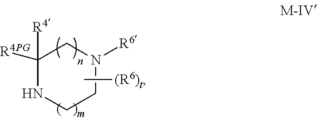

or a pharmaceutically acceptable salt thereof, wherein:
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R⁶ is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   61. A compound, wherein the compound has the structure of formula M-VI:

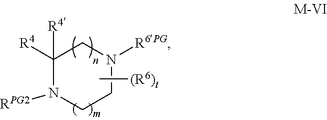

or a pharmaceutically acceptable salt thereof, wherein:
m is 1, or 2;
n is 1, or 2;
R^{PG2} is an amino protecting group;
R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R⁵ and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   62. A compound having the structure of formula M-VII:

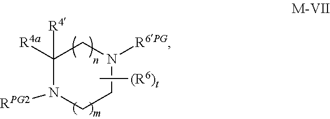

or a salt thereof, wherein:
m is 1, or 2;
n is 1, or 2;
R^{PG2} is an amino protecting group;
R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5a} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   63. A compound having the structure of formula M-VIII:

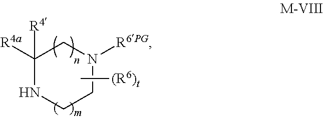

or a salt thereof, wherein:
m is 1, or 2;
n is 1, or 2;
R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5a} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   64. A compound having the structure of formula M-IX:

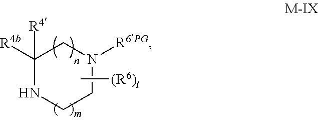

or a salt thereof, wherein:
m is 1, or 2;
n is 1, or 2;
R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5b} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5b} is —CH₂OH, —CH₂ CH₂OH, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R⁶ or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   65. A compound having the structure of formula M-X:

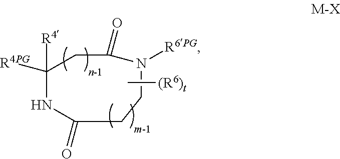

or a salt thereof, wherein:
m is 1, or 2;
n is 1, or 2;
R^{4′} is hydrogen, C₁-C₄ alkyl, or hydroxy C₁-C₄ alkyl;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG}), or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R⁶ or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   66. A compound having the structure of formula M-XI:

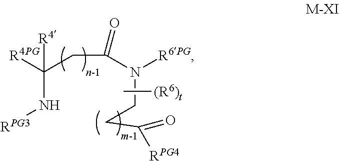

or a salt thereof, wherein:
m is 1, or 2;
n is 1, or 2;
R^{PG3} is —H or an amino protecting group;
R^{PG4} is —OH or —C(O)R^{PG4} is a protected carboxyl group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R⁵ is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R⁵ are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   67. A compound having the structure of formula M-XII:

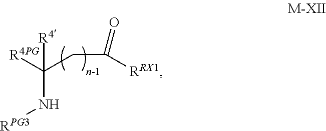

or a salt thereof, wherein:
n is 1, or 2;
R^{PG3} is —H or an amino protecting group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
R^{RX1} is —OH or —C(O)R^{RX1} is an activated carboxyl group;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   68. A compound having the structure of formula M-XIII:

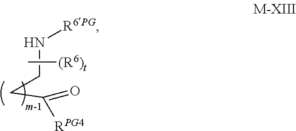

or a salt thereof, wherein:
m is 1, or 2;
R^{PG4} is —OH or —C(O)R^{PG4} is a protected carboxyl group;
R^{6′PG} is R^{6′} or an amino protecting group;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
t is 0, 1, 2, 3 or 4; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   69. A compound having the structure of formula M-XIV:

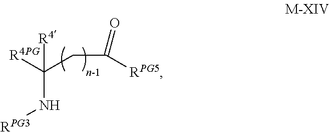

or a salt thereof, wherein:
n is 1, or 2;
R^{PG3} is —H or an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
—C(O)R^{PG5} is protected carboxyl;
R^{4′} is hydrogen, C₁-C₄ alkyl, or hydroxy C₁-C₄ alkyl;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups.
   70. A compound, wherein the compound has the structure of formula M-XV:

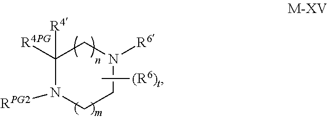

or a pharmaceutically acceptable salt thereof, wherein:
R^{PG2} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   71. A compound, wherein the compound has the structure of formula M-XVI:

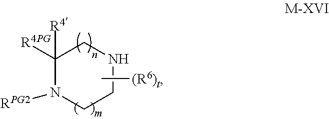

or a pharmaceutically acceptable salt thereof, wherein:
R^{PG2} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG}), or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R⁴ is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   72. A compound, wherein the compound has the structure of formula M-XVII:

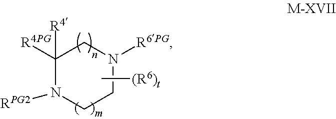

or a pharmaceutically acceptable salt thereof, wherein:
R^{PG2} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   73. The compound of any one of the preceding Embodiments, wherein R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R⁵ and further optionally substituted with one or two R⁵′.
   74. The compound of any one of the preceding Embodiments, wherein R⁴ is phenyl substituted with R⁵ and further optionally substituted with one or two R⁵′.
   75. The compound of any one of the preceding Embodiments, wherein R⁴ is phenyl para-substituted (unless otherwise indicated, relative to the atom at which R⁴ is attached to the rest of the compound) with R⁵ and further optionally substituted with one or two R⁵′.
   76. The compound of any one of the preceding Embodiments, wherein R⁴ is phenyl meta-substituted with R⁵ and further optionally substituted with one or two R⁵′.
   77. The compound of any one of Embodiments 1-72, wherein R⁴ is naphthyl substituted with R⁵ and further optionally substituted with one or two R⁵′.
   78. The compound of any one of Embodiments 1-72, wherein R⁴ is 1,2,3,4-tetrahydronaphthalenyl substituted with R⁵ and further optionally substituted with one or two R⁵′.
   79. The compound of any one of Embodiments 1-72, wherein R⁴ is 5-membered heteroaryl substituted with R⁵ and further optionally substituted with one or two R⁵′.
   80. The compound of any one of Embodiments 1-72, wherein R⁴ is 6-membered heteroaryl substituted with R⁵ and further optionally substituted with one or two R⁵′.
   81. The compound of any one of Embodiments 1-72, wherein R⁴ is 6-membered heteroaryl para-substituted with R⁵ and further optionally substituted with one or two R⁵′.
   82. The compound of any one of Embodiments 1-72, wherein R⁴ is 6-membered heteroaryl meta-substituted with R⁵ and further optionally substituted with one or two R⁵′.
   83. The compound of any one of Embodiments 80-82, wherein the 6-membered heteroaryl is pyridyl.
   84. The compound of any one of Embodiments 1-72, wherein R⁴ is 9-membered bicyclic heteroaryl substituted with R⁵ and further optionally substituted with one or two R⁵′.
   85. The compound of any one of Embodiments 1-72, wherein R⁴ is 10-membered bicyclic heteroaryl substituted with R⁵ and further optionally substituted with one or two R⁵′.
   86. The compound of any one of the preceding Embodiments, wherein R⁵ is —C(O)R⁸.
   87. The compound of any one of the preceding Embodiments, wherein R⁵ is —CH₂C(O)R⁸.
   88. The compound of any one of the preceding Embodiments, wherein R⁸ is hydroxy.
   89. The compound of any one of the preceding Embodiments, wherein R⁸ is C₁-C₄ alkoxy.
   90. The compound of any one of the preceding Embodiments, wherein R⁸ is —NH₂.
   91. The compound of any one of the preceding Embodiments, wherein R⁸ is 5-7 membered saturated heterocyclyl having 1, 2, or 3 ring heteroatoms independently selected from nitrogen, oxygen and sulfur.
   92. The compound of any one of the preceding Embodiments, wherein R⁸ is mono- and di-C₁-C₄ alkylamino which is optionally substituted with one or more substituents independently selected from halogen, hydroxy and C₁-C₄ alkyl.
   93. The compound of any one of Embodiments 1-85, wherein R⁵ is —C(O)NHSO₂C₁-C₄alkyl.
   94. The compound of any one of Embodiments 1-85, wherein R⁵ is —SO₂NHC(O)C₁-C₄alkyl.
   95. The compound of any one of Embodiments 1-85, wherein R⁵ is —SO₂N(H)ₚ(C₁-C₄alkyl)₂₋ₚ.
   96. The compound of any one of Embodiments 1-85, wherein R⁵ is —SO₂NH₂.
   97. The compound of any one of Embodiments 1-85, wherein R⁵ is —SO(NH)C₁-C₄alkyl.
   98. The compound of any one of Embodiments 1-85, wherein R⁵ is —SO(NH)CH₃.
   99. The compound of any one of Embodiments 1-85, wherein R⁵ is —SO₂C₁-C₄alkyl.
   100. The compound of any one of Embodiments 1-85, wherein R⁵ is cyano.
   101. The compound of any one of Embodiments 1-85, wherein R⁵ is halogen.
   102. The compound of any one of Embodiments 1-85, wherein R⁵ is hydroxy C₁-C₄ alkyl.
   103. The compound of any one of Embodiments 1-85, wherein R⁵ is —B(R⁸)₂.
   104. The compound of Embodiment 103, wherein each R⁸ is independently hydroxy or C₁-C₄ alkoxy.
   105. The compound of Embodiment 103, wherein each R⁸ is independently hydroxy.
   106. The compound of Embodiment 103, wherein each R⁸ is independently C₁-C₄ alkoxy.
   107. The compound of any one of Embodiments 1-85, wherein R⁵ is 5-membered heteroaryl having 1-4 ring nitrogen atoms and 0 or 1 ring oxygen or sulfur atom.
   108. The compound of Embodiment 107, wherein R⁵ is tetrazolyl.
   109. The compound of any one of the preceding Embodiments, wherein R⁴ is substituted with one R⁵′.
   110. The compound of any one of the preceding Embodiments, wherein R⁴ is substituted with two R⁵′.
   111. The compound of any one of the preceding Embodiments, wherein R⁵′ is ortho relative to R⁵.
   112. The compound of any one of the preceding Embodiments, wherein R⁵′ is halogen.
   113. The compound of any one of the preceding Embodiments, wherein R⁵′ is —F.
   114. The compound of any one of the preceding Embodiments, wherein R⁵′ is —Cl.
   115. The compound of any one of Embodiments 1-111, wherein R⁵′ is C₁-C₄ alkyl.
   116. The compound of any one of Embodiments 1-111, wherein R⁵′ is methyl.
   117. The compound of any one of Embodiments 1-111, wherein R⁵′ is ethyl.
   118. The compound of any one of Embodiments 1-111, wherein R⁵′ is cyclopropyl.
   119. The compound of any one of Embodiments 1-111, wherein R⁵′ is C₁-C₄ alkoxy.
   120. The compound of any one of Embodiments 1-111, wherein R⁵′ is —OCH₃.
   121. The compound of any one of Embodiments 1-111, wherein R⁵′ is hydroxy C₁-C₄ alkyl.
   122. The compound of any one of Embodiments 1-111, wherein R⁵′ is —CH₂OH.
   123 The compound of any one of Embodiments 1-111, wherein R⁵′ is hydroxy.
   124. The compound of any one of Embodiments 1-111, wherein R⁵′ is cyanomethyl.
   125. The compound of any one of Embodiments 1-108, wherein R⁴ is not substituted with R^{5′.}
   126. The compound of any one of Embodiments 1-111, wherein R⁵′ is —OR^{6′.}
   127. The compound of any one of Embodiments 1-111, wherein R⁵′ is —OR¹⁰.
   128. The compound of any one of Embodiments 1-111, wherein R⁵′ is R¹⁰.
   129. The compound of Embodiment 126, wherein R¹⁰ is bonded to R⁴ at a nitrogen atom.
   130. The compound of any one of Embodiments 127-129, wherein R¹⁰ is optionally substituted 3-6 membered heterocyclyl.
   131. The compound of any one of Embodiments 127-129, wherein R¹⁰ is optionally substituted 5-6 membered heteroaryl.
   132. The compound of any one of Embodiments 127-131, wherein R¹⁰ has an oxygen ring atom.
   133. The compound of Embodiment 111, wherein two R^{5′} are taken together with their intervening atoms to form a 4-8 membered monocyclic partially unsaturated or aromatic ring.
   134. The compound of Embodiment 111, wherein two R^{5′} are taken together with their intervening atoms to form a 4-8 membered monocyclic partially unsaturated ring.
   135. The compound of Embodiment 111, wherein two R^{5′} are taken together with their intervening atoms to form a 5-6 membered monocyclic heteroaryl ring.
   136. The compound of any one of Embodiments 1-111, wherein R⁵′ is —N(R⁵¹)₂.
   137. The compound of any one of Embodiments 1-111, wherein R⁵′ is —NHR⁵¹.
   138. The compound of any one of Embodiments 136-137, wherein each R⁵¹ is independently hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
   139. The compound of any one of Embodiments 136-137, wherein an occurrence of R⁵¹ is hydrogen.
   140. The compound of any one of Embodiments 136-137, wherein an occurrence of R⁵¹ is optionally substituted C₁-C₆ aliphatic.
   141. The compound of any one of Embodiments 136-137, wherein an occurrence of R⁵¹ is C₁-C₆ aliphatic.
   142. The compound of any one of Embodiments 136-137, wherein an occurrence of R⁵¹ is C₁-C₆ alkyl.
   143. The compound of any one of Embodiments 136-137, wherein an occurrence of R⁵¹ is cyclopropyl.
   144. The compound of any one of Embodiments 136-137, wherein an occurrence of R⁵¹ is methyl.
   145. The compound of any one of Embodiments 136-137, wherein an occurrence of R⁵¹ is ethyl.
   146. The compound of any one of Embodiments 136-137, wherein an occurrence of R⁵¹ is cyclobutyl.
   147. The compound of any one of Embodiments 136-137, wherein an occurrence of R⁵¹ is —CH₂-cyclopropyl.
   148. The compound of any one of Embodiments 136-137, wherein an occurrence of R⁵¹ is substituted C₁-C₆ aliphatic.
   149. The compound of any one of Embodiments 136-137, wherein an occurrence of R⁵¹ is substituted C₁-C₆ alkyl.
   150. The compound of any one of Embodiments 136-137, wherein an occurrence of R⁵¹ is (S)—CH(CH₃)(CF₃).
   151. The compound of any one of Embodiments 136-137, wherein an occurrence of R⁵¹ is —CH₂CF₃.
   152. The compound of any one of Embodiments 136-137, wherein an occurrence of R⁵¹ is substituted C₃-C₆ cycloalkyl.
   153. The compound of any one of Embodiments 136-137, wherein an occurrence of R⁵¹ is 3,3-difluorocyclobutyl.
   154. The compound of any one of Embodiments 136-137, wherein an occurrence of R⁵¹ is R¹⁰.
   155. The compound of any one of Embodiments 136-137, wherein an occurrence of R⁵¹ is —CH₂—R¹⁰.
   156. The compound of any one of Embodiments 154-155, wherein R¹⁰ is optionally substituted 5-6 membered heteroaryl having 1-3 ring atoms independently selected from nitrogen, oxygen and sulfur.
   157. The compound of any one of Embodiments 154-155, wherein R¹⁰ is 5-membered heteroaryl having 1-3 ring nitrogen atoms substituted with C₁-C₄ alkyl.
   158. The compound of any one of Embodiments 154-155, wherein R¹⁰ is

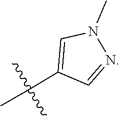 [SMILES: *c1cnn(C)c1]

159. The compound of any one of Embodiments 154-155, wherein R¹⁰ is optionally substituted 3-6 membered heterocyclyl.
160. The compound of any one of Embodiments 154-155, wherein R¹⁰ is optionally substituted 6-membered heterocyclyl.
161. The compound of Embodiment 159, wherein R¹⁰ is

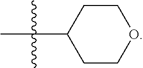 [SMILES: *C1CCOCC1]

162. The compound of Embodiment 159, wherein R¹⁰ is

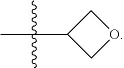 [SMILES: *C1COC1]

163. The compound of any one of Embodiments 136-137, wherein an occurrence of R⁵¹ is —C(O)—C₁-C₆ aliphatic, wherein the C₁-C₆ aliphatic is optionally substituted.
164. The compound of any one of Embodiments 136-137, wherein an occurrence of R⁵¹ is —C(O)—C₁-C₆ aliphatic.
165. The compound of any one of Embodiments 136-137, wherein an occurrence of R⁵¹ is —C(O)—C₁-C₆ alkyl.
166. The compound of any one of Embodiments 136-137, wherein R⁵′ is —NHC(O)CH₃.
167. The compound of any one of Embodiments 136-137, wherein R⁵′ is —NHC(O)CH₂CH₃.
168. The compound of any one of Embodiments 136-137, wherein an occurrence of R⁵¹ is —SO₂—C₁-C₆ aliphatic, wherein the C₁-C₆ aliphatic is optionally substituted.
169. The compound of any one of Embodiments 136-137, wherein an occurrence of R⁵¹ is —SO₂—C₁-C₆ aliphatic.
170. The compound of any one of Embodiments 136-137, wherein an occurrence of R⁵¹ is —SO₂—C₁-C₆ alkyl.
171. The compound of any one of Embodiments 136-137, wherein R⁵′ is —NHS(O)₂CH₃.
172. The compound of Embodiment 136, wherein R⁵′ is —NH₂.
173. The compound of Embodiment 136, wherein R⁵′ is —NH(CH₃).
174. The compound of Embodiment 136, wherein R⁵′ is —NH(CH₂CH₃).
175. The compound of any one of the preceding Embodiments, wherein the carbon to which R⁴ is attached has a configuration of

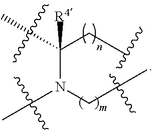

176. The compound of any one of Embodiments 1-174, wherein the carbon to which R⁴ is attached has a configuration of

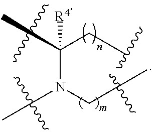

177. The compound of any one of the preceding Embodiments, wherein R^{4′} is hydrogen.
178. The compound of any one of the preceding Embodiments, wherein t is 1, 2, 3 or 4.
179. The compound of any one of the preceding Embodiments, wherein an occurrence of R⁶ is hydroxy.
180. The compound of any one of the preceding Embodiments, wherein an occurrence of R⁶ is amino.
181. The compound of any one of the preceding Embodiments, wherein an occurrence of R⁶ is mono- and di-C₁-C₄ alkylamino.
182. The compound of any one of the preceding Embodiments, wherein an occurrence of R⁶ is C₁-C₆ alkyl.
183. The compound of any one of the preceding Embodiments, wherein an occurrence of R⁶ is C₁-C₄ alkyl.
184. The compound of any one of the preceding Embodiments, wherein an occurrence of R⁶ is methyl.
185. The compound of any one of the preceding Embodiments, wherein an occurrence of R⁶ is halo C₁-C₆ alkyl.
186. The compound of any one of the preceding Embodiments, wherein an occurrence of R⁶ is halo C₁-C₄ alkyl.
187. The compound of any one of the preceding Embodiments, wherein an occurrence of R⁶ is —CF₃.
188. The compound of any one of the preceding Embodiments, wherein an occurrence of R⁶ is —CHF₂.
189. The compound of any one of the preceding Embodiments, wherein an occurrence of R⁶ is —CH₂F.
190. The compound of any one of the preceding Embodiments, wherein an occurrence of R⁶ is hydroxy C₁-C₆ alkyl.
191. The compound of any one of the preceding Embodiments, wherein an occurrence of R⁶ is hydroxy C₁-C₄ alkyl.
192. The compound of any one of the preceding Embodiments, wherein an occurrence of R⁶ is —CH₂OH.
193. The compound of any one of the preceding Embodiments, wherein an occurrence of R⁶ is cyano C₁-C₆ alkyl.
194. The compound of any one of the preceding Embodiments, wherein an occurrence of R⁶ is cyano C₁-C₄ alkyl.
195. The compound of any one of the preceding Embodiments, wherein an occurrence of R⁶ is
C₁-C₆ alkoxy.
196. The compound of any one of the preceding Embodiments, wherein an occurrence of R⁶ is C₁-C₄ alkoxy.
197. The compound of any one of the preceding Embodiments, wherein two R⁶ on a carbon atom are taken together to form ═O.
198. The compound of any one of the preceding Embodiments, wherein two R⁶ on a carbon atom are taken together to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
199. The compound of any one of the preceding Embodiments, wherein two R⁶ on a carbon atom are taken together to form a cyclopropyl ring.
200. The compound of any one of the preceding Embodiments, wherein two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
201. The compound of any one of the preceding Embodiments, wherein two R⁶ are taken together with their intervening atoms to form a 5-7 membered cycloalkyl ring, wherein the ring is optionally substituted with one or more substituents independently selected from halogen, C₁-C₄ alkyl, halo C₁-C₄ alkyl, C₁-C₄ alkoxy, hydroxyl, and oxo.
202. The Embodiment of any one of the preceding Embodiments, wherein m is 1.
203. The compound of any one of Embodiments 1-201, wherein m is 2.
204. The Embodiment of any one of the preceding Embodiments, wherein n is 1.
205. The compound of any one of Embodiments 1-203, wherein n is 2.
206. The compound of any one of Embodiments 1-201, wherein the compound has the structure of:

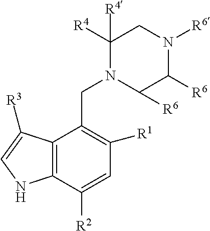

or a pharmaceutically acceptable salt thereof.
207. The compound of Embodiment 206, wherein an occurrence of R⁶ is hydrogen.
208. The compound of any one of Embodiments 206-207, wherein each R⁶ is independently selected from —H, —CH₂OH, —CH₂F, —CHF₂ and —CF₃.
209. The compound of any one of Embodiments 1-201, wherein the compound has the structure of:

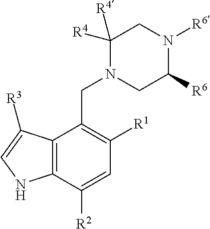

or a pharmaceutically acceptable salt thereof.
210. The compound of any one of Embodiments 1-201, wherein the compound has the structure of:

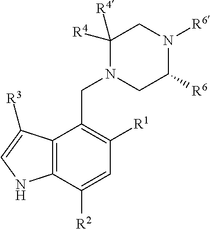

or a pharmaceutically acceptable salt thereof.
211. The compound of any one of Embodiments 1-201, wherein the compound has the structure of:

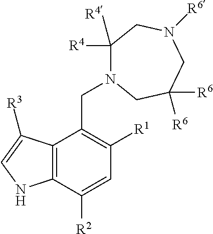

or a pharmaceutically acceptable salt thereof.
212. The compound of any one of Embodiments 1-201, wherein the compound has the structure of:

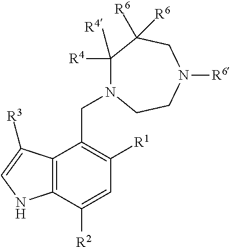

or a pharmaceutically acceptable salt thereof.
213. The compound of any one of Embodiments 211-212, wherein an occurrence of R⁶ is hydrogen.
214. The compound of any one of Embodiments 211-213, wherein an occurrence of R⁶ is —OH.
215. The compound of any one of Embodiments 211-213, wherein an occurrence of R⁶ is halogen.
216. The compound of any one of Embodiments 211-213, wherein an occurrence of R⁶ is —F.
217. The compound of any one of Embodiments 211-213, wherein an occurrence of R⁶ is C₁₋₆ alkyl.
218. The compound of any one of Embodiments 211-213, wherein an occurrence of R⁶ is methyl.
219. The compound of any one of Embodiments 211-212, wherein each R⁶ is independently halogen.
220. The compound of any one of Embodiments 211-212, wherein each R⁶ is independently —F.
221. The compound of any one of Embodiments 1-201, wherein t is 2, 3 or 4.
222. The compound of any one of Embodiments 1-201, wherein t is 2.
223. The compound of any one of the preceding Embodiments, wherein at least one occurrence of R⁶ is bonded to a carbon atom that is bonded to the nitrogen atom to which R^{6′} is bonded to.
224. The compound of Embodiment 223, wherein the carbon atom is not bonded to the carbon atom to which R⁴ is bonded to.
225. The compound of Embodiment 223, wherein the carbon atom is bonded to the carbon atom to which R⁴ is bonded to.
226. The compound of any one of Embodiments 1-177, wherein t is 0.
227. The compound of any one of Embodiments 1-196, wherein t is 1.
228. The compound of any one of Embodiments 1-177, wherein the compound has the structure of:

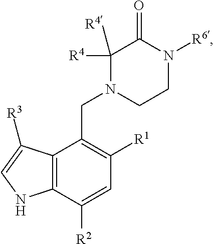

or a pharmaceutically acceptable salt thereof.
229. The compound of any one of Embodiments 1-177, wherein the compound has the structure of:

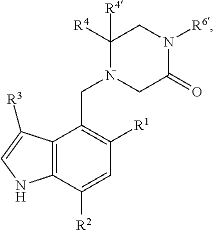

or a pharmaceutically acceptable salt thereof.
230. The compound of any one of Embodiments 1-177, wherein the compound has the structure of:

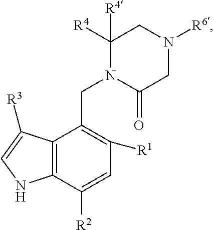

or a pharmaceutically acceptable salt thereof.
231. The compound of any one of Embodiments 1-196, wherein the compound has the structure of:

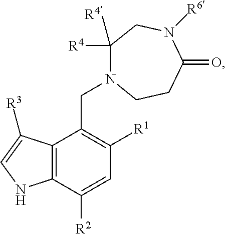

or a pharmaceutically acceptable salt thereof.
232. The compound of any one of Embodiments 1-196, wherein the compound has the structure of:

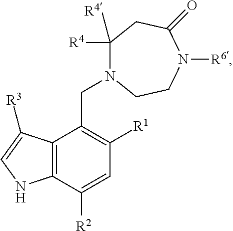

or a pharmaceutically acceptable salt thereof.
233. The compound of any one of the preceding Embodiments, wherein R^{6′} is hydrogen.
234. The compound of any one of Embodiments 1-232, wherein R^{6′} is —CH₂-(hydroxy C₁-C₄ alkyl).
235. The compound of any one of Embodiments 1-232, wherein R^{6′} is —CH₂—CH₂OH.
236. The compound of any one of Embodiments 1-232, wherein R^{6′} is —CH₂-(amino C₁-C₄ alkyl).
237. The compound of any one of Embodiments 1-232, wherein R^{6′} is —CH₂-(mono- and di-C₁-C₄ alkylamino C₁-C₄ alkyl).
238. The compound of any one of Embodiments 1-232, wherein R^{6′} is optionally substituted C₁-C₆ aliphatic.
239. The compound of any one of Embodiments 1-232, wherein R^{6′} is optionally substituted C₁-C₆ linear aliphatic.
240. The compound of any one of Embodiments 1-239, wherein R^{6′} is optionally substituted C₁-C₆ alkyl.
241. The compound of any one of Embodiments 1-239, wherein R^{6′} is optionally substituted C₁-C₄ alkyl.
242. The compound of any one of Embodiments 238-241, wherein the C₁-C₆ aliphatic is substituted with one C₁-C₄ alkoxy substituent.
243. The compound of any one of Embodiments 238-241, wherein R^{6′} is —CH₂CH₂OCH₃.
244. The compound of any one of Embodiments 1-232, wherein R^{6′} is C₁-C₆ aliphatic.
245. The compound of any one of Embodiments 1-232, wherein R^{6′} is C₁-C₆ linear aliphatic.
246. The compound of any one of Embodiments 1-245, wherein R^{6′} is C₁-C₆ alkyl.
247. The compound of any one of Embodiments 1-245, wherein R^{6′} is C₁-C₄ alkyl.
248. The compound of any one of Embodiments 1-232, wherein R^{6′} is methyl.
249. The compound of any one of Embodiments 1-232, wherein R^{6′} is ethyl.
250. The compound of any one of Embodiments 1-232, wherein R^{6′} is n-propyl.
251. The compound of any one of Embodiments 1-232, wherein R^{6′} is isopropyl.
252. The compound of any one of Embodiments 1-232, wherein R^{6′} is n-butyl.
253. The compound of any one of Embodiments 1-232, wherein R^{6′} is 2-butyl.
254. The compound of any one of Embodiments 1-232, wherein R^{6′} is —CH₂CH═CH₂.
255. The compound of any one of Embodiments 1-232, wherein R^{6′} is —CH₂CH═CH₂.
256. The compound of any one of Embodiments 1-232, wherein R^{6′} is C₁-C₄ alkoxy.
257. The compound of any one of Embodiments 1-232, wherein R^{6′} is C₃-C₆ cycloalkyl.
258. The compound of any one of Embodiments 1-232, wherein R^{6′} is —CH(CH₃)—C₁-C₄ aliphatic.
259. The compound of Embodiment 258, wherein the C₁-C₄ aliphatic is linear.
260. The compound of Embodiment 258, wherein the C₁-C₄ aliphatic is C₁-C₄ alkyl.
261. The compound of Embodiment 258, wherein the C₁-C₄ aliphatic is linear C₁-C₄ alkyl.
262. The compound of Embodiment 258, wherein the C₁-C₄ aliphatic is methyl.
263. The compound of Embodiment 258, wherein the C₁-C₄ aliphatic is ethyl.
264. The compound of Embodiment 258, wherein the C₁-C₄ aliphatic is n-propyl.
265. The compound of any one of Embodiments 1-232, wherein R^{6′} is C₃-C₆ cycloaliphatic.
266. The compound of any one of Embodiments 1-232, wherein R^{6′} is C₃-C₆ cycloalkyl.
267. The compound of any one of Embodiments 1-232, wherein R^{6′} is cyclopropyl.
268. The compound of any one of Embodiments 1-232, wherein R^{6′} is cyclobutyl.
269. The compound of any one of Embodiments 1-232, wherein R^{6′} is cyclopentyl.
270. The compound of any one of Embodiments 1-232, wherein R^{6′} is —CH₂—C₃-C₅ cycloaliphatic.
271. The compound of Embodiment 270, wherein the C₃-C₅ cycloaliphatic is C₃-C₅ cycloalkyl.
272. The compound of Embodiment 270, wherein the C₃-C₅ cycloaliphatic is C₃-C₅ cyclopropyl.
273. The compound of Embodiment 270, wherein the C₃-C₅ cycloaliphatic is C₃-C₅ cyclobutyl.
274. The compound of Embodiment 270, wherein the C₃-C₅ cycloaliphatic is C₃-C₅ cyclopentyl.
275. The compound of any one of Embodiments 1-232, wherein R^{6′} is C₁-C₆ aliphatic substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
276. The compound of any one of Embodiments 1-232, wherein R⁶ is C₁-C₆ alkyl substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
277. The compound of any one of Embodiments 275-276, wherein R^{6′} is C₁-C₆ aliphatic substituted with one or more halogen.
278. The compound of any one of Embodiments 275-277, wherein R^{6′} is C₁-C₆ alkyl substituted with one or more —F.
279. The compound of any one of Embodiments 275-277, wherein R^{6′} is C₁-C₆ linear alkyl substituted with one or more —F.
280. The compound of any one of Embodiments 275-279, wherein R^{6′} is —CH₂CH₂F.
281. The compound of any one of Embodiments 275-279, wherein R^{6′} is —CH₂CH₂CH₂F.
282. The compound of any one of Embodiments 275-279, wherein R^{6′} is —CH₂CHF₂.
283. The compound of any one of Embodiments 275-279, wherein R^{6′} is —CH₂CH₂CHF₂.
284. The compound of any one of Embodiments 275-279, wherein R^{6′} is —CH₂CF₃.
285. The compound of any one of Embodiments 275-279, wherein R^{6′} is —CH₂CH₂CF₃.
286. The compound of any one of Embodiments 275-277, wherein R^{6′} is —CH₂CF═CH₂.
287. The compound of any one of Embodiments 275-276, wherein R^{6′} is C₁-C₆ aliphatic substituted with one or more C₁-C₄ alkyl wherein the alkyl is optionally and independently substituted with one or more substituents independently selected from halogen, C₁-C₄ alkyl, halo C₁-C₄ alkyl, C₁-C₄ alkoxy, hydroxyl, and oxo.
288. The compound of any one of Embodiments 275-276, wherein R^{6′} is C₁-C₆ aliphatic substituted with one or more C₁-C₄ alkyl.
289. The compound of any one of Embodiments 275-277, wherein R^{6′} is C₁-C₆ aliphatic substituted with one or more C₁-C₄ alkoxy wherein the alkoxy is optionally and independently substituted with one or more substituents independently selected from halogen, C₁-C₄ alkyl, halo C₁-C₄ alkyl, C₁-C₄ alkoxy, hydroxyl, and oxo.
290. The compound of any one of Embodiments 275-277, wherein R^{6′} is C₁-C₆ aliphatic substituted with one or more C₁-C₄ alkoxy.
291. The compound of any one of Embodiments 289-290, wherein an occurrence of C₁-C₄ alkoxy is methoxy.
292. The compound of any one of Embodiments 275-277, wherein R^{6′} is C₁-C₆ aliphatic substituted with one or more C₃-C₆ cycloalkyl wherein the cycloalkyl is optionally and independently substituted with one or more substituents independently selected from halogen, C₁-C₄ alkyl, halo C₁-C₄ alkyl, C₁-C₄ alkoxy, hydroxyl, and oxo.
293. The compound of any one of Embodiments 275-277, wherein R^{6′} is C₁-C₆ aliphatic substituted with one or more C₃-C₆ cycloalkyl.
294. The compound of any one of Embodiments 275-277, wherein R^{6′} is C₁-C₆ aliphatic substituted with one or more 3-6 membered heterocyclyl having 1-4 ring atoms independently selected from nitrogen, oxygen and sulfur, wherein the heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
295. The compound of any one of Embodiments 275-277, wherein R^{6′} is C₁-C₆ aliphatic substituted with one or more 3-6 membered heterocyclyl having 1-4 ring atoms independently selected from nitrogen, oxygen and sulfur, wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
296. The compound of any one of Embodiments 294-295, wherein an occurrence of 3-6 membered heterocyclyl having 1-4 ring atoms independently selected from nitrogen, oxygen and sulfur is

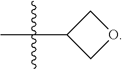 [SMILES: *C1COC1]

297. The compound of any one of Embodiments 287-296, wherein the C₁-C₆ aliphatic is linear.
298. The compound of any one of Embodiments 287-297, wherein the C₁-C₆ aliphatic is C₁-C₆ alkyl.
299. The compound of any one of Embodiments 287-298, wherein the C₁-C₆ aliphatic is ethyl.
300. The compound of Embodiment 299, wherein R^{6′} is —CH₂CH₂OCH₃.
301. The compound of any one of Embodiments 287-298, wherein the C₁-C₆ aliphatic is methyl.

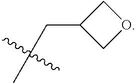 [SMILES: *CC1COC1]

302. The compound of Embodiment 301, wherein R^{6′} is
303. The compound of any one of Embodiments 1-232, wherein R^{6′} is R¹⁰.
304. The compound of Embodiment 303, wherein R¹⁰ is 5-6 membered heteroaryl having a nitrogen ring atom, wherein the heteroaryl is optionally substituted with one or more substituents independently selected from halogen, C₁-C₄ alkyl, halo C₁-C₄ alkyl, C₁-C₄ alkoxy, hydroxyl, and oxo, and wherein the nitrogen ring atom of the heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
305. The compound of Embodiment 303, wherein R¹⁰ is 5-6 membered heteroaryl having a nitrogen ring atom, wherein the nitrogen ring atom of the heteroaryl is substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
306. The compound of Embodiment 303, wherein R¹⁰ is 3-6 membered heterocyclyl having a nitrogen ring atom, wherein the heterocyclyl is optionally substituted with one or more substituents independently selected from halogen, C₁-C₄ alkyl, halo C₁-C₄ alkyl, C₁-C₄ alkoxy, hydroxyl, and oxo, and wherein the nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
307. The compound of Embodiment 303, wherein R¹⁰ is 3-6 membered heterocyclyl having a nitrogen ring atom, wherein the nitrogen ring atom of the heterocyclyl is substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
308. The compound of Embodiment 303, wherein R¹⁰ is 3-6 membered heterocyclyl having a nitrogen ring atom substituted with C₁₋₃ acyl.
309. The compound of Embodiment 303, wherein R¹⁰ is

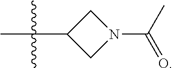 [SMILES: *C1CN(C(C)=O)C1]

310. The compound of any one of Embodiments 136-137, wherein an occurrence of R^{6′} is —SO₂—C₁-C₆ aliphatic, wherein the C₁-C₆ aliphatic is optionally substituted.
311. The compound of any one of Embodiments 136-137, wherein an occurrence of R^{6′} is —SO₂—C₁-C₆ aliphatic.
312. The compound of any one of Embodiments 136-137, wherein an occurrence of R^{6′} is —SO₂—C₁-C₆ alkyl.
313. The compound of any one of Embodiments 136-137, wherein R^{6′} is —S(O)₂CH₃.
314. The compound of any one of Embodiments 136-137, wherein an occurrence of R^{6′} is —C(O)—C₁-C₆ aliphatic, wherein the C₁-C₆ aliphatic is optionally substituted.
315. The compound of any one of Embodiments 136-137, wherein an occurrence of R⁶ is —C(O)—C₁-C₆ aliphatic.
316. The compound of any one of Embodiments 136-137, wherein an occurrence of R^{6′} is —C(O)—C₁-C₆ alkyl.
317. The compound of any one of Embodiments 136-137, wherein R^{6′} is —C(O)CH₃.
318. The compound of any one of Embodiments 1-225, wherein R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and OXO.
319. The compound of any one of Embodiments 1-225, wherein R⁶ and R^{6′} are taken together with their intervening atoms to form a 3-8 membered monocyclic or bicyclic saturated, or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
320. The compound of any one of Embodiments 1-225, wherein R⁶ and R^{6′} are taken together with their intervening atoms to form a 3-8 membered monocyclic or bicyclic saturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
321. The compound of any one of Embodiments 1-225, wherein R⁶ and R^{6′} are taken together with their intervening atoms to form a 3-8 membered monocyclic or bicyclic partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
322. The compound of any one of Embodiments 1-225, wherein R⁶ and R^{6′} are taken together with their intervening atoms to form a 5-6 membered monocyclic or bicyclic aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
323. The compound of any one of Embodiments 1-225, wherein R⁶ and R^{6′} are taken together with their intervening atoms to form a 5-8 membered monocyclic saturated or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
324. The compound of any one of Embodiments 1-225, wherein R⁶ and R^{6′} are taken together with their intervening atoms to form a 5-8 membered monocyclic saturated or partially unsaturated ring having 1 ring nitrogen atom.
325. The compound of any one of Embodiments 1-225, wherein R⁶ and R^{6′} are taken together with their intervening atoms to form a 5-8 membered monocyclic saturated ring having 1 ring nitrogen atom.
326. The compound of any one of Embodiments 1-225, wherein R⁶ and R^{6′} are taken together with their intervening atoms to form an optionally substituted 5-membered monocyclic saturated ring having 1 ring nitrogen atom.
327. The compound of Embodiment 309, wherein R⁶ and R^{6′} are taken together with their intervening atoms to form

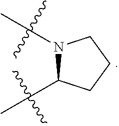 [SMILES: *[C@@H]1CCCN1*]

328. The compound of Embodiment 309, wherein R⁶ and R^{6′} are taken together with their intervening atoms to form

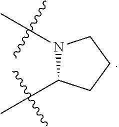 [SMILES: *[C@H]1CCCN1*]

329. The compound of Embodiment 309, wherein R⁶ and R^{6′} are taken together with their intervening atoms to form

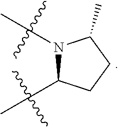 [SMILES: *[C@@H]1CC[C@@H](C)N1*]

330. The compound of Embodiment 309, wherein R⁶ and R^{6′} are taken together with their intervening atoms to form

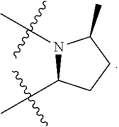 [SMILES: *[C@@H]1CC[C@H](C)N1*]

331. The compound of Embodiment 309, wherein R⁶ and R^{6′} are taken together with their intervening atoms to form

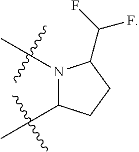 [SMILES: *C1CCC(C(F)F)N1*]

332. The compound of Embodiment 309, wherein R⁶ and R^{6′} are taken together with their intervening atoms to form

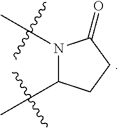 [SMILES: *C1CCC(=O)N1*]

333. The compound of any one of Embodiments 1-225, wherein R⁶ and R^{6′} are taken together with their intervening atoms to form a 5-membered monocyclic saturated ring having 1 ring nitrogen atom, wherein the ring is substituted with one or more substituents each of which is independently halogen.
334. The compound of Embodiment 333, wherein R⁶ and R^{6′} are taken together with their intervening atoms to form

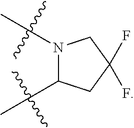 [SMILES: *C1CC(F)(F)CN1*]

335. The compound of Embodiment 333, wherein R⁶ and R^{6′} are taken together with their intervening atoms to form

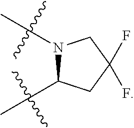 [SMILES: *[C@@H]1CC(F)(F)CN1*]

336. The compound of Embodiment 333, wherein R⁶ and R^{6′} are taken together with their intervening atoms to form

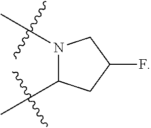 [SMILES: *C1CC(F)CN1*]

337. The compound of Embodiment 333, wherein R⁶ and R^{6′} are taken together with their intervening atoms to form

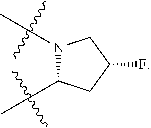 [SMILES: *[C@H]1C[C@@H](F)CN1*]

338. The compound of Embodiment 333, wherein R⁶ and R^{6′} are taken together with their intervening atoms to form

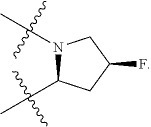 [SMILES: *[C@@H]1C[C@H](F)CN1*]

339. The compound of Embodiment 333, wherein R⁶ and R^{6′} are taken together with their intervening atoms to form

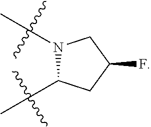 [SMILES: *[C@H]1C[C@H](F)CN1*]

340. The compound of Embodiment 333, wherein R⁶ and R^{6′} are taken together with their intervening atoms to form

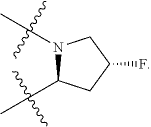 [SMILES: *[C@@H]1C[C@@H](F)CN1*]

341. The compound of any one of Embodiments 1-225, wherein R⁶ and R^{6′} are taken together with their intervening atoms to form an optionally substituted 4-membered monocyclic saturated ring having 1 ring nitrogen atom.

342. The compound of Embodiment 341, wherein R⁶ and R^{6′} are taken together with their intervening atoms to form

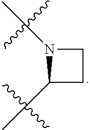 [SMILES: *[C@@H]1CCN1* ; "."]

343. The compound of Embodiment 341, wherein R⁶ and R^{6′} are taken together with their intervening atoms to form

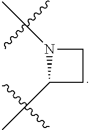 [SMILES: *[C@H]1CCN1* ; "."]

344. The compound of any one of Embodiments 1-225, wherein R⁶ and R^{6′} are taken together with their intervening atoms to form an optionally substituted 6-membered monocyclic saturated ring having 1 ring nitrogen atom.
345. The compound of Embodiment 344, wherein R⁶ and R^{6′} are taken together with their intervening atoms to form

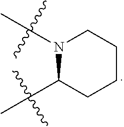 [SMILES: *[C@@H]1CCCCN1* ; "."]

346. The compound of Embodiment 344, wherein R⁶ and R^{6′} are taken together with their intervening atoms to form

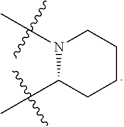 [SMILES: *[C@H]1CCCCN1* ; "."]

347. The compound of Embodiment 344, wherein R⁶ and R^{6′} are taken together with their intervening atoms to form

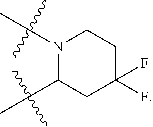 [SMILES: *C1CC(F)(F)CCN1*]

348. The compound of any one of Embodiments 1-225, wherein R⁶ and R^{6′} are taken together with their intervening atoms to form an optionally substituted 6-membered monocyclic saturated ring having 1 ring nitrogen atom and 1 ring oxygen atom.
349. The compound of Embodiment 348, wherein R⁶ and R^{6′} are taken together with their intervening atoms to form

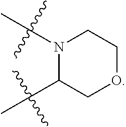 [SMILES: *C1COCCN1*]

350. The compound of any one of Embodiments 1-225, wherein R⁶ and R^{6′} are taken together with their intervening atoms to form a 6-8 membered bicyclic saturated or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
351. The compound of any one of Embodiments 1-225, wherein R⁶ and R^{6′} are taken together with their intervening atoms to form a 6-membered bicyclic saturated or partially unsaturated ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.
352. The compound of Embodiment 351, wherein R⁶ and R^{6′} are taken together with their intervening atoms to form optionally substituted

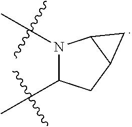 [SMILES: *C1CC2CC2N1* ; "."]

353. The compound of Embodiment 351, wherein R⁶ and R^{6′} are taken together with their intervening atoms to form optionally substituted

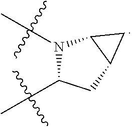 [SMILES: *[C@H]1C[C@H]2C[C@H]2N1* ; "."]

354. The compound of Embodiment 351, wherein R⁶ and R^{6′} are taken together with their intervening atoms to form optionally substituted

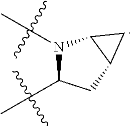 [SMILES: *[C@@H]1C[C@H]2C[C@H]2N1* ; "."]

355. The compound of Embodiment 351, wherein R⁶ and R^{6′} are taken together with their intervening atoms to form optionally substituted

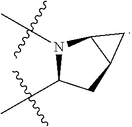 [SMILES: *[C@@H]1C[C@@H]2C[C@@H]2N1* ; "."]

356. The compound of any one of the preceding Embodiments, wherein R^{PG} is an amino protecting group.
357. The compound of any one of the preceding Embodiments, wherein R^{PG} is —C(O)-optionally substituted C₁-C₆ aliphatic.
358. The compound of any one of the preceding Embodiments, wherein R^{PG} is -Boc.
359. The compound of any one of the preceding Embodiments, wherein R^{PG2} is an amino protecting group.
360. The compound of any one of the preceding Embodiments, wherein R^{PG2} is —C(O)-optionally substituted C₁-C₆ aliphatic.
361. The compound of any one of the preceding Embodiments, wherein R^{PG2} is -Boc.
362. The compound of any one of the preceding Embodiments, wherein R^{PG3} is an amino protecting group.
363. The compound of any one of the preceding Embodiments, wherein R^{PG3} is —C(O)-optionally substituted C₁-C₆ aliphatic.
364. The compound of any one of the preceding Embodiments, wherein R^{PG3} is -Boc.
365. The compound of any one of the preceding Embodiments, wherein —C(O)R^{PG4} is a protected carboxyl group.
366. The compound of any one of the preceding Embodiments, wherein R^{PG4} is —O-optionally substituted C₁-C₆ aliphatic.
367. The compound of any one of the preceding Embodiments, wherein R^{PG4} is —O-optionally substituted C₁-C₆ alkyl.
368. The compound of any one of the preceding Embodiments, wherein R^{PG4} is —OMe.
369. The compound of any one of Embodiments 1-364, wherein R^{PG4} is —OH.
370. The compound of any one of the preceding Embodiments, wherein —C(O)R^{PG5} is a protected carboxyl group.
371. The compound of any one of the preceding Embodiments, wherein R^{PG5} is —O-optionally substituted C₁-C₆ aliphatic.
372. The compound of any one of the preceding Embodiments, wherein R^{PG5} is —O-optionally substituted C₁-C₆ alkyl.
373. The compound of any one of the preceding Embodiments, wherein R^{PG5} is —OMe.
374. The compound of any one of Embodiments 1-369, wherein R^{PG5} is —OH.
375. The compound of any one of the preceding Embodiments, R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5PG} and further optionally substituted with one or two R⁵′.
376. The compound of any one of the preceding Embodiments, R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5PG} and further substituted with 1 R⁵′.
377. The compound of any one of Embodiments 1-375, R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5PG} and further substituted with two R⁵′.
378. The compound of any one of Embodiments 1-374, wherein R^{4PG} is phenyl substituted with R^{5PG}.
379. The compound of any one of Embodiments 1-374, wherein R^{4PG} is phenyl substituted with R^{5PG} and R⁵′.
380. The compound of any one of Embodiments 1-374, wherein R^{4PG} is

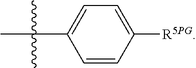

381. The compound of any one of Embodiments 1-374, wherein R^{4PG} is

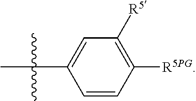

382. The compound of any one of the preceding Embodiments, wherein R^{5PG} is —C(O)R^{8PG} wherein R^{8PG} is optionally substituted C₁₋₆ aliphatic.
383. The compound of any one of the preceding Embodiments, wherein R^{5PG} is R^{5PG} is —C(O)CH₃.
384. The compound of any one of the preceding Embodiments, wherein R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5a} and further optionally substituted with one or two R⁵′.
385. The compound of any one of the preceding Embodiments, wherein R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5a} and further substituted with 1 R^{5′.}
386. The compound of any one of Embodiments 1-384, wherein R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5a} and further substituted with two R⁵′.
387. The compound of any one of Embodiments 1-383, wherein R^{4a} is phenyl substituted with R^{5a} and further optionally substituted with one or two R⁵′.
388. The compound of any one of Embodiments 1-383, wherein R^{4a} is phenyl substituted with R^{5a} and further substituted with 1 R⁵′.
389. The compound of any one of Embodiments 1-383, wherein R^{4a} is phenyl substituted with R^{5a} and further substituted with two R⁵′.
390. The compound of any one of Embodiments 1-383, wherein R^{4a} is

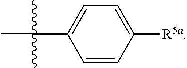

391. The compound of any one of Embodiments 1-383, wherein R^{4a} is

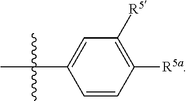

392. The compound of any one of the preceding Embodiments, wherein R^{5a} is —CHO.
393. The compound of any one of the preceding Embodiments, wherein R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5b} and further optionally substituted with one or two R⁵′.
394. The compound of any one of the preceding Embodiments, wherein R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5b} and further substituted with 1 R⁵′.
395. The compound of any one of Embodiments 1-393, wherein R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R^{5b} and further substituted with two R⁵′.
396. The compound of any one of Embodiments 1-392, wherein R^{4b} is phenyl substituted with R^{5b}.
397. The compound of any one of Embodiments 1-392, wherein R^{4b} is phenyl substituted with R^{5b} and further optionally substituted with one or two R⁵′.
398. The compound of any one of Embodiments 1-392, wherein R^{4b} is phenyl substituted with R^{5b} and further substituted with 1 R⁵′.
399. The compound of any one of Embodiments 1-392, wherein R^{4b} is phenyl substituted with R^{5b} and further substituted with two R⁵′.
400. The compound of any one of Embodiments 1-392, wherein R^{4b} is phenyl substituted with R^{5b} and further substituted with two R⁵′.
401. The compound of any one of Embodiments 1-392, wherein R^{4b} is

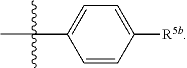

402. The compound of any one of Embodiments 1-392, wherein R^{4b} is

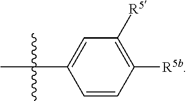

403. The compound of any one of the preceding Embodiments, wherein R^{5b} is —CH₂OH.
404. The compound of any one of the preceding Embodiments, wherein R^{6′PG} is an amino protecting group.
405. The compound of any one of the preceding Embodiments, wherein R^{6′PG} is —C(O)-optionally substituted C₁-C₆ aliphatic.
406. The compound of any one of the preceding Embodiments, wherein R^{6′PG} is -Boc.
407. The compound of any one of Embodiments 1-403, wherein R^{6′PG} is -Bn.
408. The compound of any one of the preceding Embodiments, wherein —C(O)R^{RX1} is an activated carboxyl group.
409. The compound of any one of Embodiments 1-407, wherein R^{RX1} is —OH.
410. A compound selected from Table C-1 or E-1 or a pharmaceutically acceptable salt thereof.
411. The compound of any one of the preceding Embodiments, wherein the compound has an IC50 of about or no more than about 1000, 900, 800, 700, 600, 500, 400, 300, 200, 100, 90, 80, 70, 60, 50, 40, 30, 20, or 10 nM as measured in an assay in the Examples.
412. The compound of any one of the preceding Embodiments, wherein the compound has a diastereomeric purity of about or no less than about 90%, 91%, 92%, 93%, 94%, 95%, 96%, 97%, 98%, or 99%.
413. The compound of any one of the preceding Embodiments, wherein the compound has a enantiomeric purity of about or no less than about 90%, 91%, 92%, 93%, 94%, 95%, 96%, 97%, 98%, or 99%.
414. The compound of any one of the preceding Embodiments, wherein the compound has a purity of about or no less than about 90%, 91%, 92%, 93%, 94%, 95%, 96%, 97%, 98%, or 99%.
415. A pharmaceutical composition comprising a compound of any one of the preceding Embodiments or a pharmaceutically acceptable salt thereof, and a pharmaceutically acceptable carrier, excipient or diluent.
416. A pharmaceutical composition which delivers a compound of any one of the preceding Embodiments or a pharmaceutically acceptable salt thereof.
417. A method, comprising removing one or more protection groups from a compound of formula M-I:

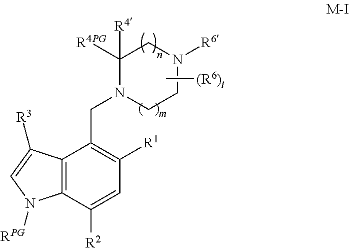

or a salt thereof to provide a compound of formula I or a salt thereof.
418. The method of Embodiment 417, wherein the compound of formula M-I or a salt thereof is a compound of any one of Embodiments 2-414.
419. The method of Embodiment 417 or 418, wherein the compound of formula I or a salt thereof is a compound of any one of Embodiments 1-414.
420. The method of any one of Embodiments 417-419, wherein R^{PG} is Boc.
421. The method of any one of Embodiments 417-420, wherein an ester in R^{4PG} is hydrolyzed to form-COOH or a salt form thereof.
422. A method comprising contacting an aldehyde or ketone with a compound of formula M-II:

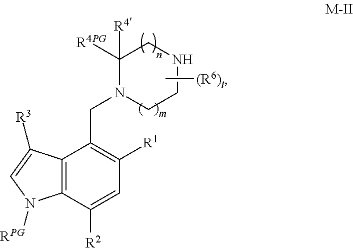

or a salt thereof in the presence of a reducing agent to provide a compound of formula M-I or a salt thereof.
423. The method of Embodiment 422, wherein the compound of formula M-I or a salt thereof is a compound of any one of Embodiments 2-414.
424. The method of Embodiment 422 or 423, wherein R⁶ in formula M-I or a salt thereof is C₁-C₆ aliphatic, R¹⁰, —CH₂—C₃-C₆ cycloaliphatic, —CH₂—R¹⁰, —CH₂-(hydroxy C₁-C₄ alkyl), —CH₂-phenyl, —CH₂-(amino C₁-C₄ alkyl), or —CH₂-(mono- and di-C₁-C₄ alkylamino C₁-C₄ alkyl), each of which is independently optionally substituted.
425. The method of any one of Embodiments 422-424, wherein an aldehyde or ketone is of such a structure that when its —C(O)— group is replaced with —CH₂—, a compound of H—R^{6′} is formed.
426. The method of any one of Embodiments 422-425, wherein the reducing agent is triacetoxyborohydride.
427. The method of any one of Embodiments 422-426, wherein the compound of formula M-II or a salt thereof is a compound of any one of Embodiments 3-414.
428. The method of any one of Embodiments 417-421, comprising a method of any one of Embodiments 422-427.
429. A method comprising reacting a compound of formula M-II:

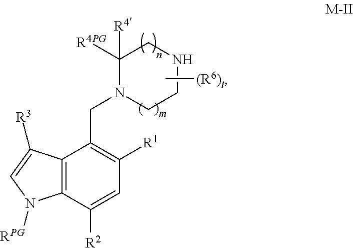

or a salt thereof with a compound having the structure of formula R^{6x3}—R⁶′, wherein R^{6x3} is a leaving group.
430. The method of Embodiment 429, wherein the compound of formula M-II or a salt thereof is a compound of any one of Embodiments 3-414.
431. The method of Embodiment 429 or 430, wherein R^{6x3} is halogen.
432. The method of any one of Embodiments 429-431, wherein R^{6x3}—R⁶ is bromobenzene.
433. The method of any one of Embodiments 429-431, wherein R^{6x3}—R^{6′} is bromobenzene.
434. The method of any one of Embodiments 429-431, wherein 1-fluoro-3-iodopropane.
435. The method of any one of Embodiments 417-421, comprising a method of any one of Embodiments 429-434.
436. A method comprising reacting a compound of formula M-II:

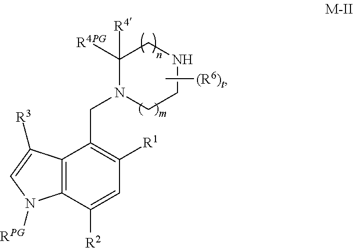

or a salt thereof with an acylating agent.
437. The method of Embodiment 436, wherein the compound of formula M-II or a salt thereof is a compound of any one of Embodiments 3-414.
438. The method of Embodiment 436 or 437, wherein an acylating agent has the structure of R^{6x1}—C(O)—C₁-C₆ aliphatic or a salt thereof, wherein R^{6x1} is —OH or R^{6x1}—C(O)— is an activated carboxyl group.
439. The method of any one of Embodiments 417-421, comprising a method of any one of Embodiments 436-438.
440. A method comprising reacting a compound of formula M-II:

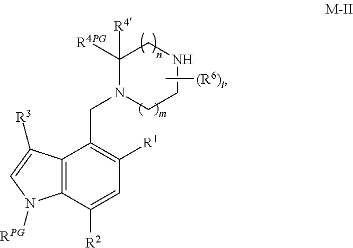

or a salt thereof with a sulfonylation agent.
441. The method of Embodiment 440, wherein the compound of formula M-II or a salt thereof is a compound of any one of Embodiments 3-414.
442. The method of Embodiment 440 or 441, wherein a sulfonylation agent has the structure of R^{6x2}—S(O)₂—C₁-C₆ aliphatic or a salt thereof, wherein R^{6x2}—S(O)₂— is an activated sulfonyl group.
443. The method of any one of Embodiments 440-442, wherein R^{6x2} is halogen.
444. The method of any one of Embodiments 440-442, wherein the sulfonylation agent is MsCl.
445. The method of any one of Embodiments 417-421, comprising a method of any one of Embodiments 440-444.
446. A method comprising de-protecting a compound having the structure of M-III:

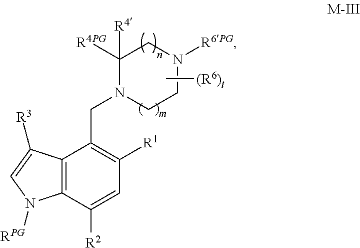

or a salt thereof to provide a compound of formula M-II or a salt thereof.
447. The method of Embodiment 446, wherein the compound of formula M-II or a salt thereof is a compound of any one of Embodiments 3-414.
448. The method of Embodiment 446 or 447, wherein the compound of formula M-III or a salt thereof is a compound of any one of Embodiments 4-414.
449. The method of any one of Embodiments 446-448, wherein R^{6′PG} is removed.
450. The method of any one of Embodiments 446-449, wherein R^{6′PG} is Bn.
451. The method of any one of Embodiments 417-445, comprising a method of any one of Embodiments 446-450.
452. A method comprising reacting a compound of formula M-IV:

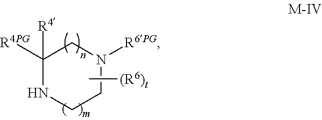

or a salt thereof, with a compound of formula M-V or a salt thereof:

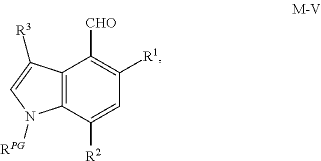

under a condition to provide a compound of formula M-III or a salt thereof.
453. The method of Embodiment 452, wherein the compound of formula M-III or a salt thereof is a compound of any one of Embodiments 4-414.
454. The method of Embodiment 452 or 453, wherein the compound of formula M-IV or a salt thereof is a compound of any one of Embodiments 59-414.
455. The method of any one of Embodiments 452-454, wherein the reaction is performed in the presence of a reducing agent.
456. The method of any one of Embodiments 417-451, comprising a method of any one of Embodiments 452-455.
457. A method comprising protecting a group and/or de-protecting a group of a compound of formula M-VI:

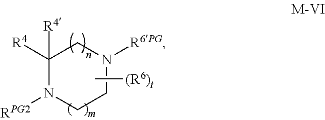

or a salt thereof to provide a compound of formula M-IV or a salt thereof.
458. The method of Embodiment 457, wherein the compound of formula M-IV or a salt thereof is a compound of any one of Embodiments 59-414.
459. The method of Embodiment 457 or 458, wherein the compound of formula M-VI or a salt thereof is a compound of any one of Embodiments 61-414.
460. The method of any one of Embodiments 457-459, wherein R^{PG2} is -Boc.
461. The method of any one of Embodiments 417-456, comprising a method of any one of Embodiments 457-460.
462. A method comprising converting a compound of formula M-VII:

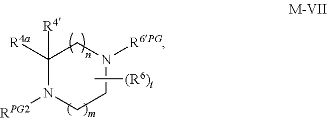

or a salt thereof to provide a compound of formula M-VI or a salt thereof.
463. The method of Embodiment 462, wherein the compound of formula M-VI or a salt thereof is a compound of any one of Embodiments 61-414.
464. The method of Embodiment 462 or 463, wherein the compound of formula M-VII or a salt thereof is a compound of any one of Embodiments 62-414.
465. The method of any one of Embodiments 462-464, comprising oxidizing a —CHO group in R^{4a} to a —COOH group.
466. The method of any one of Embodiments 417-461, comprising a method of any one of Embodiments 462-465.
467. A method comprising protecting a group in a compound of formula M-VIII:

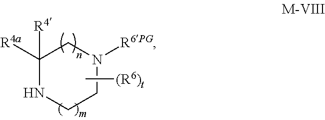

or a salt thereof to provide a compound of formula M-VII or a salt thereof.
468. The method of Embodiment 467, wherein the compound of formula M-VII or a salt thereof is a compound of any one of Embodiments 62-414.
469. The method of Embodiment 467 or 468, wherein the compound of formula M-VIII or a salt thereof is a compound of any one of Embodiments 63-414.
470. The method of any one of Embodiments 417-466, comprising a method of any one of Embodiments 467-469.
471. A method comprising converting a compound of formula M-IX:

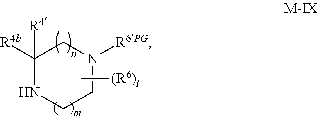

or a salt thereof to provide a compound of formula M-VIII or a salt thereof.
472. The method of Embodiment 471, wherein the compound of formula M-VIII or a salt thereof is a compound of any one of Embodiments 63-414.
473. The method of Embodiment 471 or 472, wherein the compound of formula M-IX or a salt thereof is a compound of any one of Embodiments 64-414.
474. The method of any one of Embodiments 471-473, comprising oxidizing a —CH₂OH group in R^{4b} to a —CHO group.
475. The method of any one of Embodiments 417-470, comprising a method of any one of Embodiments 471-474.
476. A method comprising reducing a compound of formula M-X:

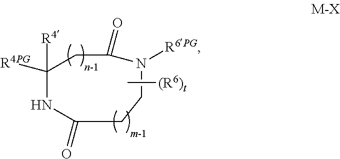

or a salt thereof to provide a compound of formula M-IX or a salt thereof.
477. The method of Embodiment 476, wherein the compound of formula M-IX or a salt thereof is a compound of any one of Embodiments 64-414.
478. The method of Embodiment 476 or 477, wherein the compound of formula M-X or a salt thereof is a compound of any one of Embodiments 65-414.
479. The method of any one of Embodiments 417-475, comprising a method of any one of Embodiments 476-478.
480. A method comprising converting a compound of formula M-XI:

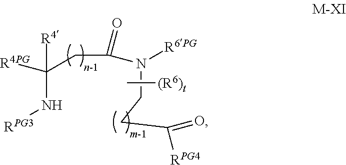

or a salt thereof to provide a compound of formula M-X or a salt thereof.
481. The method of Embodiment 480, wherein the compound of formula M-X or a salt thereof is a compound of any one of Embodiments 65-414.
482. The method of Embodiment 480 or 481, wherein the compound of formula M-XI or a salt thereof is a compound of any one of Embodiments 66-414.
483. The method of any one of Embodiments 417-479, comprising a method of any one of Embodiments 480-482.
484. A method comprising reacting a compound of formula M-XII:

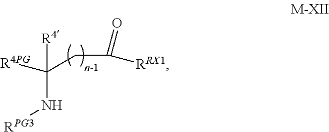

or a salt thereof, with a compound of formula M-XIII:

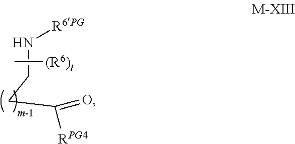

or a salt thereof to provide a compound of formula M-XI or a salt thereof.
485. The method of Embodiment 484, wherein the compound of formula M-XI or a salt thereof is a compound of any one of Embodiments 66-414.
486. The method of Embodiment 484 or 485, wherein the compound of formula M-XII or a salt thereof is a compound of any one of Embodiments 67-414.
487. The method of any one of Embodiments 484-486, wherein the compound of formula M-XIII or a salt thereof is a compound of any one of Embodiments 68-414.
488. The method of any one of Embodiments 417-483, comprising a method of any one of Embodiments 484-487.
489. A method comprising converting a compound of formula M-XIV:

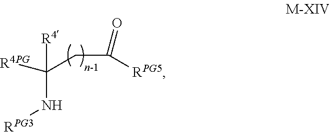

or a salt thereof to a compound of formula M-XII or a salt thereof.
490. The method of Embodiment 489, wherein the compound of formula M-XII or a salt thereof is a compound of any one of Embodiments 67-414.
491. The method of Embodiment 489 or 490, wherein the compound of formula M-XIV or a salt thereof is a compound of any one of Embodiments 69-414.
492. The method of any one of Embodiments 417-488, comprising a method of any one of Embodiments 489-491.
493. A method comprising reacting a compound of formula M-IV′:

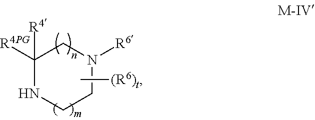

or a salt thereof, with a compound of formula M-V or a salt thereof:

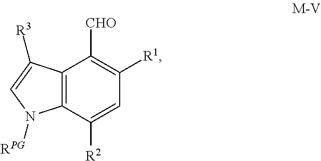

under a condition to provide a compound of formula M-I or a salt thereof.
494. The method of Embodiment 493, wherein the compound of formula M-I or a salt thereof is a compound of any one of Embodiments 2-414.
495. The method of Embodiment 493 or 494, wherein the compound of formula M-IV′ or a salt thereof is a compound of any one of Embodiments 60-414.
496. The method of any one of Embodiments 493-495, wherein the reaction is performed in the presence of a reducing agent.
497. The method of any one of Embodiments 417-492, comprising a method of any one of Embodiments 493-496.
498. A method comprising de-protecting a group of a compound of formula M-XV:

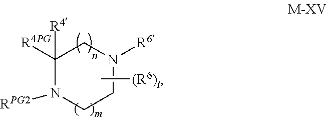

or a salt thereof to provide a compound of formula M-IV′ or a salt thereof.
499. The method of Embodiment 498, wherein the compound of formula M-IV′ or a salt thereof is a compound of any one of Embodiments 60-414.
500. The method of Embodiment 498 or 499, wherein the compound of formula M-XV or a salt thereof is a compound of any one of Embodiments 70-414.
501. The method of any one of Embodiments 417-497, comprising a method of any one of Embodiments 498-500.
502. A method comprising converting compound of formula M-XVI:

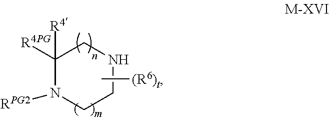

or a salt thereof to a compound of formula M-XV or a salt thereof.
503. The method of Embodiment 502, wherein the compound of formula M-XV or a salt thereof is a compound of any one of Embodiments 70-414.
504. The method of Embodiment 502 or 503, wherein the compound of formula M-XVI or a salt thereof is a compound of any one of Embodiments 71-414.
505. The method of any one of Embodiments 502-504, wherein the method is or comprises a reductive amination reaction with an aldehyde or ketone whose structure is such that when its —C(O)— is replaced with —CH₂—, a compound having the structure of R⁶—H or a salt thereof is formed.
506. The method of any one of Embodiments 502-504, wherein the method is or comprises an amination reaction.
507. The method of any one of Embodiments 502-504, wherein the method is or comprises an amination reaction with R^{6′}-boronic acid.
508. The method of any one of Embodiments 502-504, wherein the method is or comprises an acylating reaction, wherein an acylating agent has the structure of R^{6x1}—C(O)—C₁-C₆ aliphatic or a salt thereof.
509. The method of any one of Embodiments 502-504, wherein the method is or comprises an sulfonylation reaction, wherein a sulfonylation agent has the structure of R^{6x2}—S(O)₂—C₁-C₆ aliphatic or a salt thereof.
510. The method of any one of Embodiments 417-501, comprising a method of any one of Embodiments 502-509.
511. A method comprising de-protecting a group of a compound of formula M-XVII:

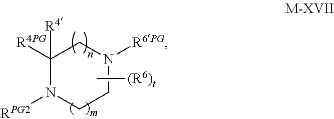

or a salt thereof to a compound of formula M-XVI or a salt thereof.
512. The method of Embodiment 511, wherein the compound of formula M-XVI or a salt thereof is a compound of any one of Embodiments 71-414.
513. The method of Embodiment 511 or 512, wherein the compound of formula M-XVII or a salt thereof is a compound of any one of Embodiments 72-414.
514. The method of any one of Embodiments 417-510, comprising a method of any one of Embodiments 511-513.
515. A method comprising protecting a group of a compound of formula M-IV or a salt thereof to provide a compound of formula M-XVII or a salt thereof.
516. The method of Embodiment 515, wherein the compound of formula M-XVII or a salt thereof is a compound of any one of Embodiments 72-414.
517. The method of Embodiment 515 or 516, wherein the compound of formula M-IV or a salt thereof is a compound of any one of Embodiments 59-414.
518. The method of any one of Embodiments 417-514, comprising a method of any one of Embodiments 515-517.
519. A method, comprising:
reacting a compound having the structure of formula M-XIII or a salt thereof with a compound having the structure of formula M-XII′:

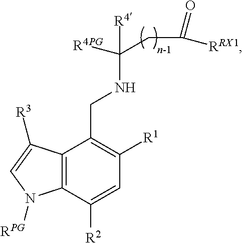

or a salt thereof to provide a compound of formula M-X′:

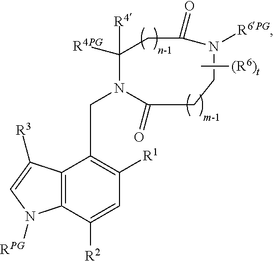

or a salt thereof.
520. The method of Embodiment 519, wherein a compound of formula M-XIII or a salt thereof is a compound of any one of Embodiments 68-414.
521. The method of any one of Embodiments 519-520, wherein a compound of formula M-XII′ or a salt thereof is a compound of any one of Embodiments 5-414.
522. The method of any one of Embodiments 519-521, wherein a compound of formula M-X′ or a salt thereof is a compound of any one of Embodiments 6-414.
523. The method of any one of Embodiments 519-522, comprising converting a compound of formula M-X′ or a salt thereof to a compound of formula M-X″ or a salt thereof.
524. The method of Embodiment 523, wherein a compound of formula M-X″ or a salt thereof is a compound of any one of Embodiments 7-414.
525. The method of any one of Embodiments 417-518, comprising a method of any one of Embodiments 519-524.
526. A method for assessing a compound, comprising utilizing an assay described in the Examples.
527. A method of inhibiting a C3 convertase, comprising contacting a C3 convertase with a compound or composition of any one of the preceding Embodiments.
528. A method of modulating complement alternative pathway activity in a subject, wherein the method comprises administering to the subject a therapeutically effective amount of a compound or composition of any one of the preceding Embodiments.
529. A method of treating a disorder or a disease in a subject mediated by complement activation, wherein the method comprises administering to the subject a therapeutically effective amount of a compound or composition of any one of the preceding Embodiments.
530. A method of treating a disorder or a disease in a subject mediated by activation of the complement alternative pathway, wherein the method comprises administering to the subject a therapeutically effective amount of a compound or composition of any one of the preceding Embodiments.
531. The method of any one of Embodiments 527-530, wherein the subject is suffering from a disease or disorder selected from age-related macular degeneration, geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.
532. The method of Embodiment 531, wherein the condition, disorder or disease is hemolytic anemia, and the subject is suffering from paroxysmal nocturnal hemoglobinuria.
533. The method of Embodiment 531, wherein the condition, disorder or disease is hemolytic anemia, and the subject is suffering from autoimmune hemolytic anemia (e.g., cold agglutinin disease or warm autoimmune hemolytic anemia).
534. The method of any one of Embodiments 527-530, wherein the subject suffers from TMA secondary to atypical hemolytic uremic syndrome.
535. The method of any one of Embodiments 527-530, wherein the subject suffers from TMA secondary to hematopoietic stem cell transplant (HSCT-TMA).
536. The method of any one of Embodiments 527-530, wherein the subject suffers from drug-induced TMA.
537. The method of any one of Embodiments 527-530, wherein the subject suffers from complement activation secondary to administration of another therapeutic or diagnostic agent.
538. The method of any one of Embodiments 527-530, wherein the subject suffers from complement-mediated disorder is complement activation secondary to gene therapy (e.g., gene therapy with a viral vector such as an adeno-associated virus (AAV), adenovirus, or lentivirus vector).
539. The method of any one of Embodiments 527-530, wherein the subject suffers from complement-mediated disorder is complement activation secondary to cell therapy.
540. A method of treating age related macular degeneration, comprising administering to a subject suffering therefrom a therapeutically effective amount of a compound or composition of any one of the preceding Embodiments.
541. The method of Embodiment 540, wherein the age related macular degeneration is intermediate age-related macular degeneration.
542. The method of any one of Embodiments 527-541, wherein another therapeutic agent is administered such that a subject is exposed to the effects of both the compound and the another therapeutic agent.
543. The method of any one of Embodiments 527-542, wherein another therapeutic agent is administered prior to, concurrently with or subsequently to the administration of the compound.

### EXEMPLIFICATION

Technologies of the present disclosure may be understood more readily by reference to the following detailed description of certain examples.

### Example 1. Preparation of Compounds—Certain Useful Procedurals.

Those skilled in the art appreciate that various technologies are available for preparing provided compounds and compositions in accordance with the present disclosure. For example, in some embodiments, technologies described below were utilized to prepare certain compounds.

In some embodiments, certain general experimental procedures are utilized. Some of such procedures are presented below as examples.

### General Experimental Procedure 1: Suzuki Reaction

A suspension of 2-chloropyrazine (1.05 eq), boronic acid or boronic ester (1.0 eq.), Pd(PPh₃)₄ (1 mol %) in dioxane (20V) was bubbled with N₂ for 30 min. Then 1.0 M aq. Na₂CO₃ (1.0 eq) was added and the mixture heated at 80° C. until LCMS showed full conversion (typically 3-5 h). The reaction was worked-up; cooled to room temperature and diluted with EtOAc (50V). The organic layer was washed with H₂O (2×10V), brine (10V), dried over Na₂SO₄, filtered, and concentrated under reduced pressure. The residue was triturated with TBME (10V), filtered, and dried under reduced pressure to afford the desired product.

### General Experimental Procedure 2: Pyrazine Reduction

Pyrazine from General Experimental Procedure 1 (1.0 eq) was dissolved in AcOH (25V) and purged with N². Pd(OAc)₂ (10 mol %) was added, the mixture purged with H₂ and then stirred under H₂ pressure (balloon) at 20° C. for 15 h. The mixture was filtered over Celite® and the filtrate concentrated under reduced pressure. The residue was suspended in H₂O (10V) and the pH adjusted to 10 with 1 M aq. NaOH. The mixture was extracted with EtOAC (2×10V) and the pooled organic layers were dried over Na₂SO₄, filtered, and concentrated under reduced pressure. The residue was purified by automated normal phase FCC to afford the desired product.

### General Experimental Procedure 3: Cbz Protection

A solution of piperazine from General Experimental Procedure 2 (1.0 eq) and DIPEA (2.0 eq.) under N₂ in CH₃CN (10V) was cooled to 0° C. Cbz-Cl (0.95 eq) was added dropwise with vigorous stirring. The reaction mixture was stirred at 0° C. for 2 h. The reaction was worked-up; diluted with H₂O (10V), removed CH₃CN under reduced pressure, and the aqueous residue was extracted with DCM (2×5V). The pooled organic layers were washed with sat. aq. NaHCO₃ (5V), dried over Na₂SO₄, filtered, and concentrated under reduced pressure. The resulting residue was purified by automated normal phase FCC to afford the desired product.

### General Experimental Procedure 4: Reductive Amination I

To a solution of Cbz protected piperazine from General Experimental Procedure 3 (1.0 eq.) in THF (15V) were added tert-butyl 4-formyl-5-methoxy-7-methyl-1H-indole-1-carboxylate (1.50 eq.) and AcOH (1.1 eq.). The resulting mixture was stirred at 20° C. for 1 h before NaBH(OAc)₃ (4.0 eq.) was added and the reaction mixture further stirred at 20° C. for 15 h. The reaction was worked-up; concentrated under reduced pressure and the residue partitioned between DCM (10V) and sat. aq. NaHCO₃ (10V). The layers were separated and the aqueous layer extracted with DCM (2×10V). The combined organic layers were dried over Na₂SO₄, filtered, and concentrated under reduced pressure. The residue was purified by automated normal phase FCC to afford the desired product.

### General Experimental Procedure 5: Cbz Deprotection

A suspension of product from General Experimental Procedure 4 (1.0 eq) and 10% Pd/C (0.1 eq) in TFE (10V) was purged with H₂ and stirred under H₂ atmosphere (balloon) at 20° C. for 1 h. The reaction was worked-up; purged with N₂ and filtered over Celite®. The filtrate was concentrated under reduced pressure and the residue purified by automated normal phase FCC to afford the desired product.

### General Experimental Procedure 6: Reductive Amination II

To a solution of product from General Experimental Procedure 5 (1.0 eq.) in MeOH (25V) were added 37 wt. % aq. formaldehyde (6.0 eq.) and NaBH(OAc)₃ (5.0 eq.). The resulting mixture was stirred at 20° C. for 3 h. The reaction was worked-up; concentrated under reduced pressure and the residue partitioned between DCM (25V) and sat. aq. NaHCO₃ (15V). The layers were separated and the aqueous layer extracted with DCM (2×10V). The combined organic layers were dried over Na₂SO₄, filtered, and concentrated under reduced pressure to afford the desired product.

### General Experimental Procedure 7: Alkylation

To a solution of product from General Experimental Procedure 5 (1.0 eq.) under N₂ in dry THF (25V) were added Et₃N (4.0 eq.) and alkylating reagent (3.0 eq.). The reaction mixture was stirred at 20° C. for 15 h. The reaction was worked-up; concentrated under reduced pressure and the crude residue used as such in the next step.

### General Experimental Procedure 8: Boc Deprotection and Ester Hydrolysis

Protected compound (1 eq.) was dissolved in EtOH (20V), 15 wt. % aq. NaOH (10 eq) was added, and the mixture stirred at 45° C. for 15 h. The reaction mixture was neutralized to pH 7 with the addition of 37 wt. % aq. HCl and purified by automated reverse phase FCC to afford the desired product.

### General Experimental Procedure 9: Ugi Reaction and Diketopiperazine Formation

A solution of methyl 4-formylbenzoate (1.0 eq) and (5-methoxy-7-methyl-1-tosyl-1H-indol-4-yl) methanamine (1.0 eq.) in TFE (0.5 M) was stirred at 20° C. for 2 h. Then amino acid (1.0 eq.) and 2-(2,2-dimehtoxyethyl)-N-methylynebenzaminium (1.0 eq.) were added, and the mixture stirred at 20° C. for 15 h. The reaction was worked-up; concentrated under reduced pressure and the residue was purified by automated normal phase FCC to afford the desired Ugi intermediate. To the Ugi intermediate was added a mixture of DCM/TFA (2:1, 0.1 M) and the resulting solution stirred at 20° C. for 1 h. The reaction mixture was concentrated under reduced pressure and the residue co-evapoarted with DCM (2×). The resulting residue was dissolved in EtOAc (0.1 M) and Et₃N (50 eq.) was added. The resulting mixture was stirred at 20° C. for 15 h. The reaction was worked-up; concentrated under reduced pressure and the residue was purified by automated normal phase FCC to afford the desired product. In some instances, diastereomeric isomers were separated.

### General Experimental Procedure 10: Piperazine Formation

To a solution of diketopiperazine (1.0 eq.) in dry and degassed THF (50 mM) were added acetylacetonato(1,5-cyclooctadiene)rhodium (0.2 eq.) and phenylsilane (8.0 eq.) The resulting mixture was stirred at 20° C. for 15 h. The reaction was worked-up; quenched with the addition of MeOH (1.0 mL) and concentrated under reduced pressure. The residue was dissolved in THF (2.5 mL) and purified on an Isolute® SCX-2 catch and release column to afford the desired product.

### General Experimental Procedure 11: Tosyl Deprotection and Ester Hydrolysis

To a solution of protected piperazine (1.0 eq.) in THF (50 mM) was added 1.0 M TBAF in THF (3.0 eq.) and the mixture stirred at 100° C. until LCMS analysis showed full conversion (typically 15 h). The solvent was concentrated under reduced pressure, the residue dissolved in EtOAc and washed thoroughly with water. The organic layer was dried over Na₂SO₄, filtered, and concentrated under reduced pressure. The residue was converted following General Experimental Procedure 8.

LCMS: In some embodiments, LCMS was utilized to purify or characterize provided compounds or compositions. Certain useful technologies are presented below as examples.

In some embodiments, an instrument employed was the Water Acquity H-Class with PDA detector and QDA mass detector equipped with a Waters XBridge BEH C18 (2.1×50 mm, 2.5 μm; PN 186006029) column. In some embodiments, a mass detector employed the atmosphere pressure ionization via electrospray for both positive and negative conjugates (100-600 Da or 100-1000 Da if mass≥600 Da).

### LCMS Method “General 2 Basic”

Mobile phase A: 10 mM ammonium bicarbonate in water (pH 9.5)
Mobile phase B: Acetonitrile
Pump Flow: 0.6 ml/min
UV Detection: Max plot and 254 nm
Injection Volume: 0.4 μl
Run Time: 3.0 min
Column Temperature: 30° C.
Pump Program: Gradient

| Time (min) | % A | % B |
|---|---|---|
| 0.0 | 50 | 50 |
| 1.5 | 5 | 95 |
| 2.5 | 5 | 95 |
| 2.6 | 85 | 15 |
| 3.0 | 85 | 15 |

### LCMS Method “General 3 Basic”

Mobile phase A: 10 mM ammonium bicarbonate in water (pH 9.5)
Mobile phase B: Acetonitrile
Pump Flow: 0.6 ml/min
UV Detection: Max plot and 254 nm
Injection Volume: 0.4 μl
Run Time: 3.0 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |
| 3.0 | 85 | 15 |

### LCMS Method “General 2 Acidic”

Mobile phase A: 0.1% (v/v) aq. trifluoroacetic acid
Mobile phase B: Acctonitrile
Pump Flow: 0.6 ml/min
UV Detection: Max plot and 254 nm
Injection Volume: 0.4 μl
Run Time: 3.0 min
Column Temperature: 30° C.
Pump Program: Gradient

| Time (min) | % A | % B |
|---|---|---|
| 0.0 | 50 | 50 |
| 1.5 | 5 | 95 |
| 2.5 | 5 | 95 |
| 2.6 | 85 | 15 |
| 3.0 | 85 | 15 |

### LCMS Method “General 3 Acidic”

Mobile phase A: 0.1% (v/v) aq. trifluoroacetic acid
Mobile phase B: Acetonitrile
Pump Flow: 0.6 ml/min
UV Detection: Max plot and 254 nm
Injection Volume: 0.4 μl
Run Time: 3.0 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |
| 3.0 | 85 | 15 |

In some embodiments, an instrument employed was the Agilent 1260 Infinity II series with UV detector, ELSD 1290 infinity II detector and Agilent 6130 mass detector equipped with a Waters XBridge BEH C18 (2.1×50 mm, 2.5 μm; PN 186006029) column. In some embodiments, a mass detector employed the atmosphere pressure ionization via electrospray for both positive and negative conjugates (80-1200 Da).

### LCMS Method “LCMS-4 General 2 A B”

Mobile phase A: 10 mM ammonium acetate (water/methanol/acetonitrile 900/60/40)
Mobile phase B: 10 mM ammonium acetate (water/methanol/acetonitrile 100/540/360)
Pump Flow: 0.6 ml/min
UV Detection: 215 nm and 254 nm
Injection Volume: 0.3 μl
Run Time: 3.5 min
Post Time: 2.0 min
Column Temperature: 35° C.
ELSD: Evaporator temperature: 40° C.
   Nebulizer temperature: 40° C.
Pump Program: Gradient

| Time (min) | % A | % B |
|---|---|---|
| 0.0 | 50 | 50 |
| 1.5 | 0 | 100 |
| 3.0 | 0 | 100 |

### LCMS Method “LCMS-4 General 3 AB”

Mobile phase A: 10 mM ammonium acetate (water/methanol/acetonitrile 900/60/40)
Mobile phase B: 10 mM ammonium acetate (water/methanol/acetonitrile 100/540/360)
Pump Flow: 0.6 ml/min
UV Detection: 215 nm and 254 nm
Injection Volume: 0.3 μl
Run Time: 3.0 min
Post Time: 1.5 min
Column Temperature: 35° C.
ELSD: Evaporator temperature: 40° C.
   Nebulizer temperature: 40° C.
Pump Program: Gradient

| Time (min) | % A | % B |
|---|---|---|
| 0.0 | 80 | 20 |
| 1.5 | 0 | 100 |
| 3.0 | 0 | 100 |

In some embodiments, an instrument employed was the Agilent 1290 Infinity II series with UV detector, ELSD 1290 infinity II detector and Agilent 6135 mass detector equipped with a Waters XSelect SCH C18 (2.1×50 mm, 2.5 μm; PN 186006101) column. In some embodiments, a mass detector employed the atmosphere pressure ionization via electrospray for both positive and negative conjugates (100-1000 Da).

### LCMS Method “LCMS-24 Libraries”

Mobile phase A: 10 mM aq. ammonium bicarbonate, pH 9.5
Mobile phase B: Acetonitrile
Pump Flow: 0.6 ml/min
UV Detection: 210-320 nm and 215 nm
Injection Volume: 0.3 μl
Run Time: 2.7 min
Post Time: 0.5 min
Column Temperature: 25° C.
ELSD: Evaporator temperature: 80° C.
Nebulizer temperature: 80° C.
Pump Program: Gradient

| Time (min) | % A | % B |
|---|---|---|
| 0.0 | 95 | 5 |
| 2.0 | 2 | 98 |
| 2.7 | 2 | 98 |

In some embodiments, an instrument employed was the Agilent 1290 series with UV detector and HP 6130 MSD mass detector equipped with a Waters XBridge BEH C18 XP (2.1×50 mm, 2.5 μm) column. In some embodiments, a mass detector employed the atmosphere pressure ionization via electrospray for both positive and negative conjugates.

### LCMS Method “31697 LCMS-5 C3.M”

The method employed was as follows:
Mobile phase A: Ammonium acetate (10 mM) in water/methanol/acetonitrile (900:60:40)
Mobile phase B: Ammonium acetate (10 mM) in water/methanol/acetonitrile (100:540:360)
Pump Flow: 0.6 ml/min
UV Detection: 215 nm
Injection Volume: 0.1 μl
Run Time: 3.5 min
Column Temperature: 35° C.
Pump Program: Gradient

| Time (min) | % A | % B |
|---|---|---|
| 0.0 | 95 | 5 |
| 0.5 | 95 | 5 |
| 2.5 | 0 | 100 |
| 3.5 | 0 | 100 |

### LCMS Method “31697 IC Gradient”

Column: Chiralpak IC (100×4.5 mm, 5.0 μm)
Mobile phase A: CO₂
Mobile phase B: 0.2% NH₄OH in Methanol
Pump Flow: 2.5 ml/min
UV Detection: 210-320 nm
Injection Volume: 2.0 μl
Run Time: 10.0 min
Column Temperature: 40° C.
Pump Program: Gradient

| Time (min) | % A | % B | Curve |
|---|---|---|---|
| Initial | 98 | 2 | Initial |
| 3.0 | 50 | 50 | 6 |
| 10.0 | 50 | 50 | 6 |

SFC: In some embodiments, SFC was utilized to purify or characterize provided compounds or compositions. Certain useful technologies are presented below as examples.

### Preparative SFC.

In some embodiments, an instrument employed was the Water SFC prep system with PDA detector.

### SFC Method “31697-1176-09”

Column: Chiralpak IG (20×250 mm; 5.0 μm)
Mobile phase A: CO₂
Mobile phase B: 0.2% NH₄OH in Methanol
Pump Flow: 100 ml/min
PDA Detection: 236 nm
Sample concentration: 25 mg/mL in methanol
Injection Volume: 250 ML
Run Time: 8.5 min
Column Temperature: 40° C.
Pump Program: Isocratic A/B 70:30

In some embodiments, an instrument employed was the Sepiatec 250 SFC prep system with UV detector.

### SFC Method “100 mL 5% B”

Column: Lux Cellulose-1 (21.20×250 mm; 5.0 μm)
Mobile phase A: CO₂
Mobile phase B: Isopropanol/Acetonitrile/Et₂NH (50/50/0.1)
Pump Flow: 100 ml/min
UV Detection: 236 nm
Sample concentration: 25 mg/mL in methanol
Injection Volume: 300 ML
Run Time: 14 min
Column Temperature: 42° C.
Pump Program: Isocratic A/B 95:5

### SFC Method “120 mL 15% B”

Column: Lux Cellulose-1 (21.20×250 mm; 5.0 μm)
Mobile phase A: CO₂
Mobile phase B: MeOH+0.2% NH₄OH
Pump Flow: 120 ml/min
UV Detection: 224 nm
Sample concentration: 25 mg/mL in methanol
Injection Volume: 200 μL
Run Time: 8 min
Column Temperature: 42° C.
Pump Program: Isocratic A/B 85:15

In some embodiments, an instrument employed was the Sepiatec 250 SFC prep system with UV detector.

SFC Method “iCel-5 Iso40”
Column: Lux iCellulose (21.2×250 mm; 5.0 μm)
Mobile phase A: CO₂
Mobile phase B: 0.2% NH₄OH in Methanol
Pump Flow: 100 ml/min
UV Detection: 240 nm
Sample concentration: 25 mg/mL in methanol
Injection Volume: 500 μL
Run Time: 3.5 min
Column Temperature: 40° C.
Pump Program: Isocratic A/B 60:40

In some embodiments, an instrument employed was the Sepiatec 250 SFC prep system with UV detector.

### SFC Method “100 mL 20% A”

Column: Lux Cellulose-2 (21.20×250 mm; 5.0 μm)
Mobile phase A: CO₂
Mobile phase B: Methanol
Pump Flow: 100 ml/min
UV Detection: 224 nm
Sample concentration: 21.1 mg/mL in methanol
Injection Volume: 500 μL
Run Time: 4.5 min
Column Temperature: 42° C.
Pump Program: Isocratic A/B 80:20

### Analytical SFC.

In some embodiments, an instrument employed was the Water Acquity UPC² with UV detector and QDA detector. In some embodiments, a mass detector employed the electrospray for both positive and negative conjugates (e.g., 100-1250 Da).

### SFC Method “31697 J UPC2 6m Cel2 MA1”

Column: Acquity UPC² Trefoil CEL2 (3.0×150 mm; 2.5 μm)
Mobile phase A: CO₂
Mobile phase B: 0.2% ammonia in methanol
Pump Flow: 2.0 ml/min
UV Detection: 236 nm
Injection Volume: 1.0 μl
Run Time: 6.0 min
Column Temperature: 40° C.
ABPR: 2000 psi
Pump Program: Gradient

| Time (min) | % A | % B | Curve |
|---|---|---|---|
| Initial | 98 | 2 | Initial |
| 3.0 | 60 | 40 | 3 |
| 5.0 | 60 | 40 | 6 |
| 5.1 | 98 | 2 | 6 |

### SFC Method “31697V UPC2 7m Cell MA1”

Column: Acquity UPC² Trefoil CEL1 (3.0×150 mm; 2.5 μm)
Mobile phase A: CO₂
Mobile phase B: 0.2% ammonia in methanol
Pump Flow: 2.5 ml/min
UV Detection: 215 nm
Injection Volume: 1.0 μl
Run Time: 7.0 min
Column Temperature: 40° C.
ABPR: 2000 psi
Pump Program: Gradient

| Time (min) | % A | % B | Curve |
|---|---|---|---|
| Initial | 98 | 2 | Initial |
| 6.0 | 75 | 25 | 6 |
| 6.1 | 98 | 2 | 6 |

### SFC Method “31697M UPC2 10m IF MA1”

Column: Daicel Chiralpak (3.0×150 mm; 3.0 μm)
Mobile phase A: CO₂
Mobile phase B: 0.2% ammonia in methanol
Pump Flow: 1.0 ml/min
UV Detection: 238 nm
Injection Volume: 1.0 μl
Run Time: 10.0 min
Column Temperature: 40° C.
ABPR: 2000 psi
Pump Program: Gradient

| Time (min) | % A | % B | Curve |
|---|---|---|---|
| Initial | 98 | 2 | Initial |
| 6.0 | 80 | 20 | 6 |
| 9.0 | 80 | 20 | 6 |
| 9.1 | 98 | 2 | 6 |

### SFC Method “31697AD UPC2 5m Cel3 M”

Column: Phenomenex Lux Cellulose 3 (3.0×150 mm; 3.0 μm)
Mobile phase A: CO₂
Mobile phase B: Methanol
Pump Flow: 2.0 ml/min
UV Detection: 224 nm
Injection Volume: 1.0 μl
Run Time: 5.0 min
Column Temperature: 40° C.
ABPR: 2000 psi
Pump Program: Gradient

| Time (min) | % A | % B | Curve |
|---|---|---|---|
| Initial | 98 | 2 | Initial |
| 4.0 | 85 | 15 | 6 |
| 4.1 | 98 | 2 | 6 |

In some embodiments, an instrument employed was the Water Acquity UPC² with UV detector and QDA detector. In some embodiments, a mass detector employed the electrospray for both positive and negative conjugates (e.g., 100-650 Da).

### Example 2. 4-(4-(2,2-Difluoroethyl)-1-((5-methoxy-7-methyl-1H-indol-4-yl)methyl)piperazin-2-yl)-2-ethylbenzoic acid

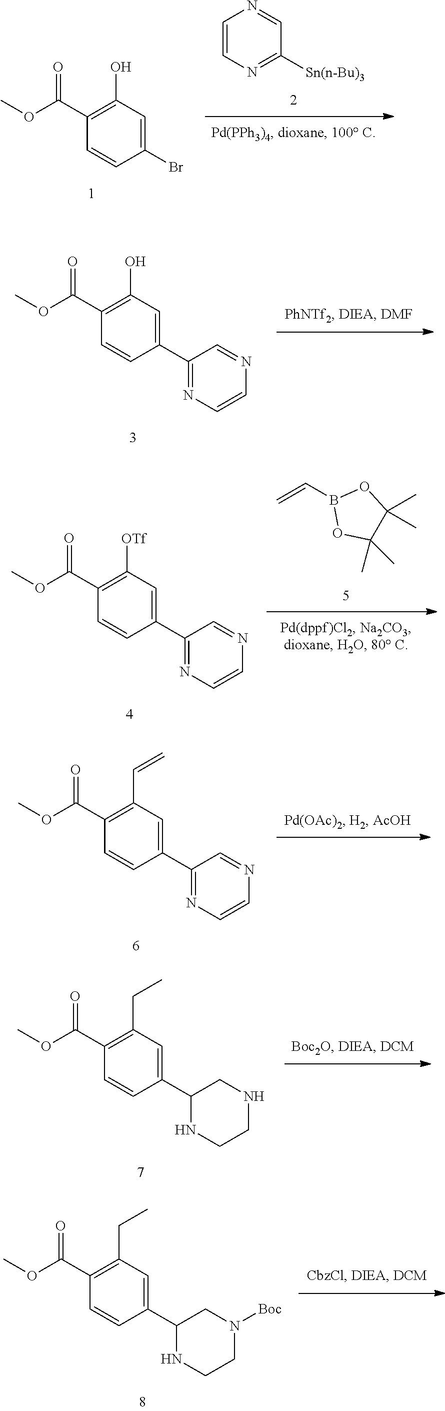

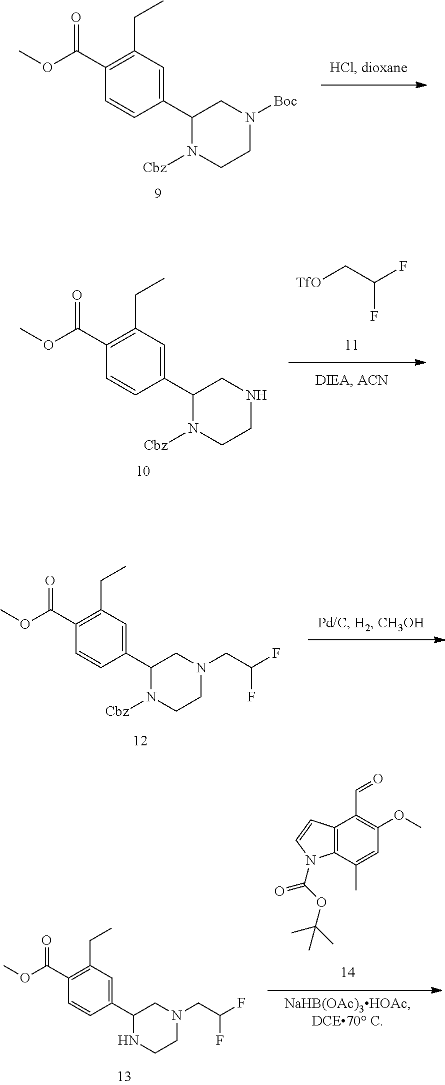 [SMILES: CCc1cc(C2CN(C(=O)OC(C)(C)C)CCN2C(=O)OCc2ccccc2)ccc1C(=O)OC ; CCc1cc(C2CNCCN2C(=O)OCc2ccccc2)ccc1C(=O)OC ; O=S(=O)(OCC(F)F)C(F)(F)F ; CCc1cc(C2CN(CC(F)F)CCN2C(=O)OCc2ccccc2)ccc1C(=O)OC ; CCc1cc(C2CN(CC(F)F)CCN2)ccc1C(=O)OC ; COc1cc(C)c2c(ccn2C(=O)OC(C)(C)C)c1C=O]

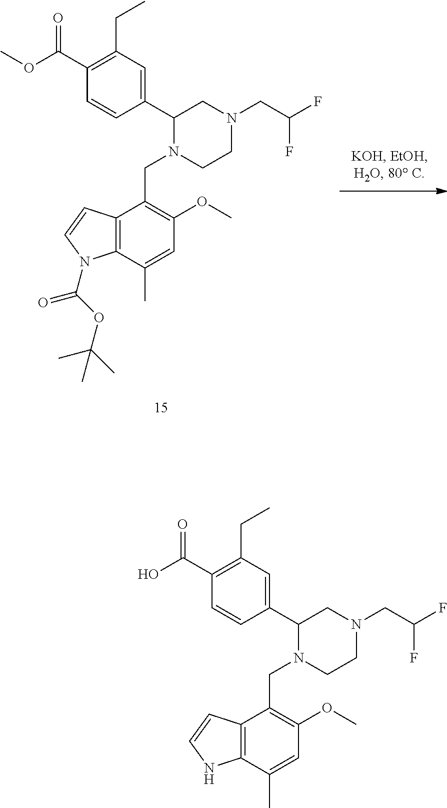 [SMILES: CCc1cc(C2CN(CC(F)F)CCN2Cc2c(OC)cc(C)c3c2ccn3C(=O)OC(C)(C)C)ccc1C(=O)OC ; CCc1cc(C2CN(CC(F)F)CCN2Cc2c(OC)cc(C)c3[nH]ccc23)ccc1C(=O)O]

Step 1: Methyl 2-hydroxy-4-(pyrazin-2-yl)benzoate. Under nitrogen, a solution of methyl 4-bromo-2-hydroxybenzoate (500 mg, 2.16 mmol), 2-(tributylstannyl)pyrazine (700 mg, 1.89 mmol) and tetrakis(triphenylphosphine)palladium (350 mg, 0.30 mmol) in dioxane (5 mL) was stirred for 2 h at 100° C. The reaction was cooled to rt and quenched with saturated potassium fluoride solution. The resulting mixture was extracted with ethyl acetate for 3 times. The combined organic layers were dried over anhydrous sodium sulfate. After filtration, the filtrate was concentrated under reduced pressure. The residue was purified by silica gel column chromatography eluting with PE/EA (4/1) to afford the title compound (300 mg, 68.7% yield) as a white solid. LCMS (ESI): [M+H]⁺=231.1.

Step 2: Methyl 4-(pyrazin-2-yl)-2-(trifluoromethanesulfonyloxy)benzoate. To a solution of methyl 2-hydroxy-4-(pyrazin-2-yl)benzoate (1 g, 4.34 mmol) in DMF (10 mL) was added 1,1,1-trifluoro-N-phenyl-N-((trifluoromethyl)sulfonyl)methanesulfonamide (1.86 g, 5.21 mmol) and N,N-diisopropylethylamine (1.68 g, 13.03 mmol) at rt. The resulting mixture was stirred for 2 h at rt. The resulting mixture was quenched with water and extracted with ethyl acetate for 3 times. The combined organic layers were washed with brine, dried over anhydrous sodium sulfate. After filtration, the filtrate was concentrated under reduced pressure. The residue was purified by silica gel column chromatography eluting with PE/EA (5/1) to afford the title compound (1.5 g, 95.3% yield) as a yellow solid. LCMS (ESI): [M+H]⁺=363.0.

Step 3: Methyl 2-ethenyl-4-(pyrazin-2-yl)benzoate. Under nitrogen, a mixture of methyl 4-(pyrazin-2-yl)-2-(trifluoromethanesulfonyloxy)benzoate (1.5 g, 4.14 mmol), 2-ethenyl-4,4,5,5-tetramethyl-1,3,2-dioxaborolane (750 mg, 4.86 mmol), (1,1′-bis(diphenylphosphino)ferrocene)dichloropalladium(II) (454 mg, 0.62 mmol) and sodium carbonate (1.3 g, 12.26 mmol) in 1,4-dioxane (10 mL) and water (2 mL) was stirred for 2 h at 80° C. The resulting mixture was quenched with water and extracted with ethyl acetate for 3 times. The combined organic layers were washed with brine, dried over anhydrous sodium sulfate. After filtration, the filtrate was concentrated under reduced pressure. The residue was purified by silica gel column chromatography eluting with PE/EA (9:1) to afford the title compound (950 mg, 95.5% yield) as a yellow solid. LCMS (ESI): [M+H]⁺=241.1.

Step 4: Methyl 2-ethyl-4-(piperazin-2-yl)benzoate. Under hydrogen, a mixture of methyl 2-ethenyl-4-(pyrazin-2-yl)benzoate (1 g, 4.16 mmol) and Palladium (II) acetate (500 mg, 2.22 mmol) in acetic acid (5 mL) was stirred for overnight at rt. After filtration, the filtrate was concentrated under reduced pressure to afford the title compound (700 mg, 67.7% yield) as a brown solid. The crude product would be used in the next step directly without further purification. LCMS (ESI): [M+H]⁺=249.2.

Step 5: tert-Butyl 3-(3-ethyl-4-(methoxycarbonyl)phenyl)piperazine-1-carboxylate. To a stirred mixture of methyl 2-ethyl-4-(piperazin-2-yl)benzoate (700 mg, 2.81 mmol) and di-tert-butyl dicarbonate (919 mg, 4.22 mmol) in dichloromethane (10 mL) was added triethylamine (855 mg, 8.44 mmol) in portions at rt. The resulting mixture was stirred for additional 1 h at rt. The reaction was quenched with water and extracted with ethyl acetate for 3 times. The organic layers were combined and dried over anhydrous sodium sulfate. After filtration, the filtrate was concentrated under reduced pressure. The residue was purified by silica gel column chromatography eluting with PE/EA (3:2) to afford the title compound (900 mg, 91.6% yield) as a yellow oil. LCMS (ESI): [M+H]⁺=349.2.

Step 6: 1-Benzyl 4-(tert-butyl) 2-(3-ethyl-4-(methoxycarbonyl)phenyl)piperazine-1,4-dicarboxylate. To a stirred solution of 1-Benzyl 4-(tert-butyl) 2-(3-ethyl-4-(methoxycarbonyl)phenyl)piperazine-1,4-dicarboxylate (900 mg, 2.58 mmol) and N,N-diisopropylethylamine (990 mg, 7.66 mmol) in dichloromethane (5 mL) was added benzyl chloroformate (540 mg, 3.16 mmol) dropwise at rt. The resulting mixture was stirred for additional 1 h at rt. The solvent was removed under vacuum. The residue was purified by silica gel column chromatography eluting with PE/EA (3:2) to afford the title compound (1 g, 80.2% yield) as a yellow oil. LCMS (ESI): [M+H]⁺=483.2.

Step 7: Benzyl 2-(3-ethyl-4-(methoxycarbonyl)phenyl)piperazine-1-carboxylate. A solution of 1-benzyl 4-(tert-butyl) 2-(3-ethyl-4-(methoxycarbonyl)phenyl)piperazine-1,4-dicarboxylate (1 g, 2.07 mmol) in dichloromethane (5 mL) was added 4 M HCl in 1,4-dioxane (2 mL) and stirred for 2 h at rt. The resulting mixture was concentrated under reduced pressure to afford the title compound (1.3 g) as a yellow oil. The crude product mixture was used in the next step directly without further purification. LCMS (ESI): [M+H]⁺=383.2.

Step 8: Benzyl 4-(2,2-difluoroethyl)-2-(3-ethyl-4-(methoxycarbonyl)phenyl)piperazine-1-carboxylate. To a stirred solution of benzyl 2-(3-ethyl-4-(methoxycarbonyl)phenyl)piperazine-1-carboxylate (1.2 g, 3.13 mmol) and N,N-diisopropylethylamine (1.2 g, 9.28 mmol) in acetonitrile (10 mL) were added 2,2-difluoroethyl trifluoromethanesulfonate (840 mg, 3.92 mmol) in portions at rt. The resulting mixture was stirred for additional 2 h at rt. The resulting mixture was concentrated under reduced pressure. The residue was purified by silica gel column chromatography eluting with PE/EA (4:1) to afford the title compound (800 mg, 57.1% yield) as a yellow oil. LCMS (ESI): [M+H]⁺=447.2.

Step 9: Methyl 4-(4-(2,2-difluoroethyl)piperazin-2-yl)-2-ethylbenzoate. Under hydrogen, a mixture of benzyl 4-(2,2-difluoroethyl)-2-(3-ethyl-4-(methoxycarbonyl)phenyl)piperazine-1-carboxylate (800 mg, 1.79 mmol) and 10% Pd/C (400 mg) in methanol (10 mL) was stirred for 5 h at rt. After filtration, the filtrate was concentrated under reduced pressure to afford the title compound (450 mg, 80.4% yield) as a yellow oil. The crude product was used in the next step directly without further purification. LCMS (ESI): [M+H]⁺=313.2.

Step 10: tert-Butyl 4-((4-(2,2-difluoroethyl)-2-(3-ethyl-4-(methoxycarbonyl)phenyl)piperazin-1-yl)methyl)-5-methoxy-7-methyl-1H-indole-1-carboxylate. To a stirred solution of methyl methyl 4-(4-(2,2-difluoroethyl)piperazin-2-yl)-2-ethylbenzoate (400 mg, 1.28 mmol) in 1,2-dichloroethane (5 mL) was added acetic acid (80 mg, 1.33 mmol), tert-butyl 4-formyl-5-methoxy-7-methylindole-1-carboxylate (740 mg, 2.55 mmol), sodium triacetoxyborohydride (540 mg, 2.54 mmol) at rt. The resulting mixture was stirred for overnight at 70° C. The reaction was quenched with water at rt. The resulting mixture was extracted with ethyl acetate. The combined organic layers were washed with water, dried over anhydrous sodium sulfate. After filtration, the filtrate was concentrated under reduced pressure. The residue was purified by silica gel column chromatography eluting with PE/EA (4:1) to afford the title compound (450 mg, 60.0% yield) as a yellow oil. LCMS (ESI) [M+H]⁺=586.3.

Step 11: 4-(4-(2,2-Difluoroethyl)-1-((5-methoxy-7-methyl-1H-indol-4-yl)methyl)piperazin-2-yl)-2-ethylbenzoic acid. A solution of tert-butyl 4-((4-(2,2-difluoroethyl)-2-(3-ethyl-4-(methoxycarbonyl)phenyl)piperazin-1-yl)methyl)-5-methoxy-7-methyl-1H-indole-1-carboxylate (450 mg, 0.10 mmol) in ethanol (5 mL) was added 5 M potassium hydroxide solution (1 mL). The resulting solution was stirred for 2 h at 80° C. The pH was adjusted to 8 with 2 M HCl. Most of the solvent was removed. The residue was purified by reverse phase flash chromatography eluting with methanol/10 mM ammonium bicarbonate (10%-100%) to afford the title compound (250 mg, 69.0% yield) as a white solid. LCMS (ESI): [M+H]⁺=472.2, ¹H NMR (300 MHz, DMSO-d₆) δ 10.72 (t, J=2.3 Hz, 1H), 7.66 (d, J=7.9 Hz, 1H), 7.33 (d, J=12.4 Hz, 2H), 7.13 (t, J=2.8 Hz, 1H), 6.54 (s, 1H), 6.44-6.29 (m, 1H), 6.25-5.73 (m, 1H), 3.60 (s, 3H), 3.47 (d, J=11.8 Hz, 1H), 3.36-3.16 (m, 2H), 3.12 (d, J=11.8 Hz, 1H), 2.87 (q, J=7.4 Hz, 2H), 2.75-2.45 (m, 5H), 2.34-2.26 (m, 3H), 2.22-2.00 (m, 3H), 1.09 (t, J=7.4 Hz, 3H).

### Example 3. (R)-4-(4-(2,2-difluoroethyl)-1-((5-methoxy-7-methyl-1H-indol-4-yl)methyl)piperazin-2-yl)-2-ethylbenzoic acid & (S)-4-(4-(2,2-difluoroethyl)-1-((5-methoxy-7-methyl-1H-indol-4-yl)methyl)piperazin-2-yl)-2-ethylbenzoic acid

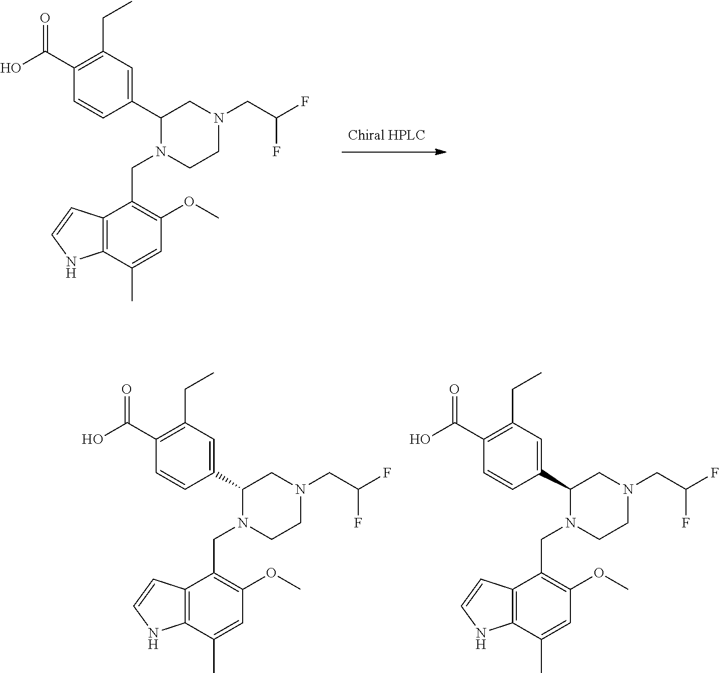 [SMILES: CCc1cc(C2CN(CC(F)F)CCN2Cc2c(OC)cc(C)c3[nH]ccc23)ccc1C(=O)O ; CCc1cc([C@@H]2CN(CC(F)F)CCN2Cc2c(OC)cc(C)c3[nH]ccc23)ccc1C(=O)O ; CCc1cc([C@H]2CN(CC(F)F)CCN2Cc2c(OC)cc(C)c3[nH]ccc23)ccc1C(=O)O]

4-(4-(2,2-difluoroethyl)-1-((5-methoxy-7-methyl-1H-indol-4-yl)methyl)piperazin-2-yl)-2-ethylbenzoic acid (225 mg, 0.48 mmol) was applied for further isolation by Chiral HPLC with the following condition to afford the compounds:

Column: CHIRALPAK ID, 2*25 cm, 5 μm; Mobile Phase A: Hex (0.1% FA)-HPLC, Mobile Phase B: IPA-HPLC; Flow rate: 20 mL/min; Gradient: 20% B to 20% B in 13 min; Wave Length: 220/254 nm; RT1(min): 6.389; RT2(min): 9.982; Sample Solvent: EtOH-HPLC; Injection Volume: 0.6 mL; Number Of Runs: 15.

(R)-4-(4-(2,2-Difluoroethyl)-1-((5-methoxy-7-methyl-1H-indol-4-yl)methyl)piperazin-2-yl)-2-ethylbenzoic acid (43.9 mg, 19.5% yield). LCMS (ESI): [M+H]⁺=472.2. ¹H NMR (300 MHz, DMSO-d₆) δ 10.72 (t, J=2.3 Hz, 1H), 7.66 (d, J=7.9 Hz, 1H), 7.33 (d, J=12.4 Hz, 2H), 7.13 (t, J=2.8 Hz, 1H), 6.54 (s, 1H), 6.44-6.29 (m, 1H), 6.25-5.73 (m, 1H), 3.60 (s, 3H), 3.47 (d, J=11.8 Hz, 1H), 3.36-3.16 (m, 2H), 3.12 (d, J=11.8 Hz, 1H), 2.87 (q, J=7.4 Hz, 2H), 2.75-2.45 (m, 5H), 2.34-2.26 (m, 3H), 2.22-2.00 (m, 3H), 1.09 (t, J=7.4 Hz, 3H). t_{R}=1.29 min (CHIRALPAK ID-3 (0.46*5 cm,3 um), Hex (0.1% FA): IPA=80:20, 1.0 mL/min, 254 nm).

(S)-4-(4-(2,2-Difluoroethyl)-1-((5-methoxy-7-methyl-1H-indol-4-yl)methyl)piperazin-2-yl)-2-ethylbenzoic acid (35.1 mg, 15.6% yield). LCMS (ESI): [M+H]⁺=472.2. ¹H NMR (300 MHz, DMSO-d₆) δ 10.72 (t, J=2.3 Hz, 1H), 7.66 (d, J=7.9 Hz, 1H), 7.33 (d, J=12.4 Hz, 2H), 7.13 (t, J=2.8 Hz, 1H), 6.54 (s, 1H), 6.44-6.29 (m, 1H), 6.25-5.73 (m, 1H), 3.60 (s, 3H), 3.47 (d, J=11.8 Hz, 1H), 3.36-3.16 (m, 2H), 3.12 (d, J=11.8 Hz, 1H), 2.87 (q, J=7.4 Hz, 2H), 2.75-2.45 (m, 5H), 2.34-2.26 (m, 3H), 2.22-2.00 (m, 3H), 1.09 (t, J=7.4 Hz, 3H). t_{R}=3.42 min (CHIRALPAK ID-3 (0.46*5 cm,3 um), Hex (0.1% FA): IPA-80:20, 1.0 mL/min, 254 nm).

### Example 4. 2-Amino-4-(4-(2,2-difluoroethyl)-1-((5-methoxy-7-methyl-1H-indol-4-yl)methyl)piperazin-2-yl)benzoic acid

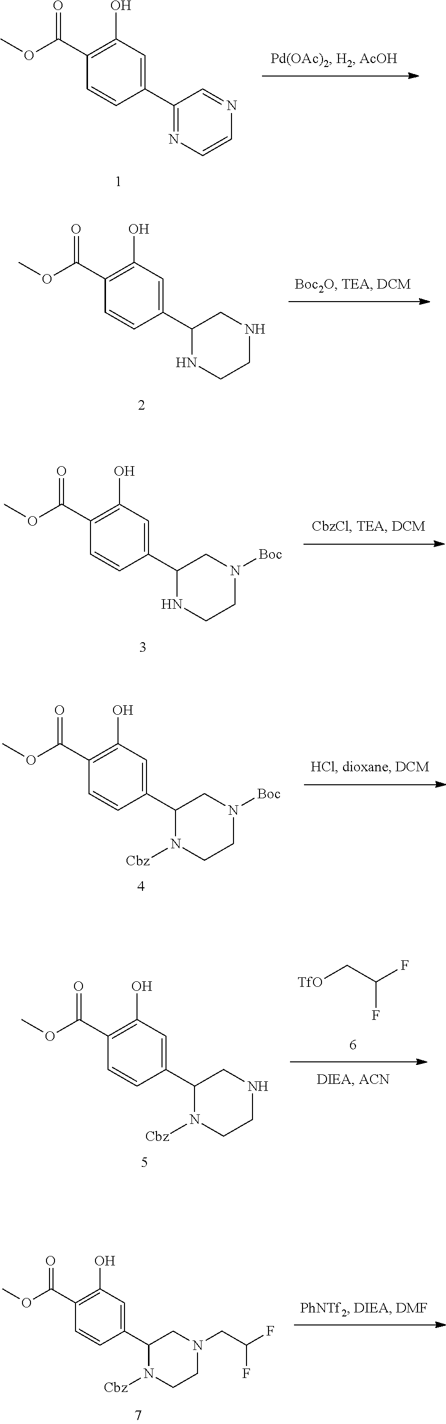

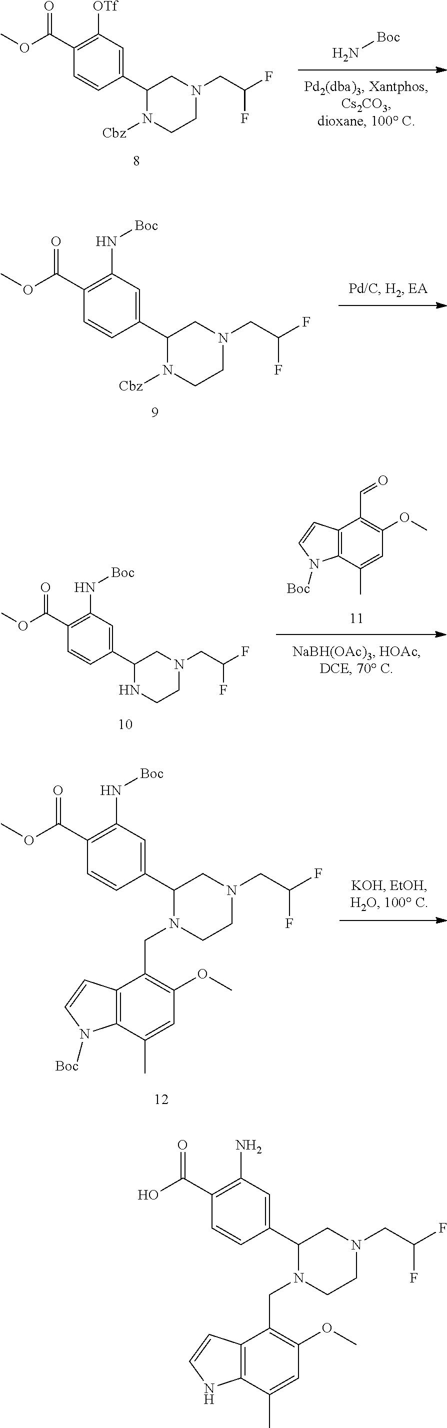

Step 1: Methyl 2-hydroxy-4-(piperazin-2-yl)benzoate. Under hydrogen, a mixture of methyl 2-hydroxy-4-(pyrazin-2-yl)benzoate (4 g, 17.37 mmol) and palladium (II) Acetate (1.9 g, 8.46 mmol) in acetic acid (20 mL) was stirred for overnight at rt. After filtration, the filtrate was concentrated under reduced pressure to afford the title compound (4.0 g) as a yellow solid. The crude product mixture was used in the next step directly without further purification. LCMS (ESI) [M+H]⁺=237.1.

Step 2: tert-Butyl 3-(3-hydroxy-4-(methoxycarbonyl)phenyl)piperazine-1-carboxylate. To a stirred solution of methyl 2-hydroxy-4-(piperazin-2-yl)benzoate (4.0 g, 16.87 mmol) and di-tert-butyl dicarbonate (4.62 g, 21.16 mmol) in DCM (40 mL) was added triethylamine (9.51 mL, 61.79 mmol) dropwise at rt. The resulting mixture was stirred for 1 h at rt. The solvent was removed under vacuum. The residue was purified by silica gel column chromatography eluting with PE/EA (3:2) to afford the title compound (5.2 g, 73.05%) as a yellow oil. LCMS (ESI) [M+H]⁺=337.1.

Step 3: 1-Benzyl 4-(tert-butyl)₂-(3-hydroxy-4-(methoxycarbonyl)phenyl)piperazine-1,4-dicarboxylate. To a stirred solution of tert-butyl 3-(3-hydroxy-4-(methoxycarbonyl)phenyl)piperazine-1-carboxylate (5.2 g, 15.4 mmol) and triethylamine (6.48 mL, 46.20 mmol) in dichloromethane (30 mL) was added benzyl chloroformate (3.95 g, 23.2 mmol) dropwise at rt. The resulting mixture was stirred for additional 1 h at room temperature. The reaction was quenched with water (50 mL) and extracted with ethyl acetate (3×50 mL). The organic layers were combined. The organic layer was washed with brine (50 mL) and dried over anhydrous sodium sulfate. After filtration, the filtrate was concentrated under vacuum. The residue was purified by silica gel column chromatography eluting with PE/EA (3:2) to afford the title compound (5.5 g, 75.8% yield) as a yellow oil. LCMS (ESI): [M+H]⁺=471.2.

Step 4: Benzyl 2-(3-hydroxy-4-(methoxycarbonyl)phenyl)piperazine-1-carboxylate. A solution of 1-benzyl 4-(tert-butyl) 2-(3-hydroxy-4-(methoxycarbonyl)phenyl)piperazine-1,4-dicarboxylate (5.5 g, 11.68 mmol) in dichloromethane (10 mL) was added 4 M HCl in 1,4-dioxane (20 mL, 80.00 mmol) at rt. The resulting solution was stirred for 3 h at rt. The resulting mixture was concentrated under reduced pressure to afford the title compound (3.7 g, 85.46%) as a yellow oil. The crude product was used in the next step directly without further purification. LCMS (ESI): [M+H]⁺=371.2.

Step 5: Benzyl 4-(2,2-difluoroethyl)-2-[3-hydroxy-4-(methoxycarbonyl)phenyl]piperazine-1-carboxylate. To a stirred mixture of benzyl 2-(3-hydroxy-4-(methoxycarbonyl)phenyl)piperazine-1-carboxylate (3.5 g, 9.44 mmol) and 2,2-difluoroethyl trifluoromethanesulfonate (2.27 g, 10.58 mmol) in acetonitrile (30 mL) was added N,N-diisopropylethylamine (3.51 g, 27.11 mmol) dropwise at rt. The resulting mixture was stirred for additional 1 h at rt. The resulting mixture was concentrated under reduced pressure. The residue was purified by silica gel column chromatography eluting with PE/EA (5:1) to afford the title compound (3.5 g, 85.26%) as a yellow oil. LCMS (ESI): [M+H]⁺=435.2.

Step 6: Benzyl 4-(2,2-difluoroethyl)-2-(4-(methoxycarbonyl)-3-(((trifluoromethyl)sulfonyl)oxy)phenyl)piperazine-1-carboxylate. To a stirred mixture of benzyl 4-(2,2-difluoroethyl)-2-[3-hydroxy-4-(methoxycarbonyl)phenyl]piperazine-1-carboxylate (3.5 g, 8.05 mmol) and 1,1,1-trifluoro-N-phenyl-N-((trifluoromethyl)sulfonyl)methanesulfonamide (3.51 g, 9.82 mmol) in DMF (20 mL) was added N,N-diisopropylethylamine (4.31 mL, 24.41 mmol) dropwise at rt. The resulting mixture was stirred for additional 2 h at rt. The reaction was quenched with water (50 mL) and extracted with ethyl acetate (3×50 mL). The organic layers were combined. The organic layer was washed with brine (50 mL) and dried over anhydrous sodium sulfate. After filtration, the filtrate was concentrated under vacuum. The residue was purified by silica gel column chromatography eluting with PE/EA (5:1) to afford the title compound (3.5 g, 76.69% yield) as a yellow oil. LCMS (ESI): [M+H]⁺=567.1.

Step 7: Benzyl 2-(3-((tert-butoxycarbonyl)amino)-4-(methoxycarbonyl)phenyl)-4-(2,2-difluoroethyl)piperazine-1-carboxylate. Under nitrogen, a mixture of benzyl 4-(2,2-difluoroethyl)-2-(4-(methoxycarbonyl)-3-(((trifluoromethyl)sulfonyl)oxy)phenyl)piperazine-1-carboxylate (1 g, 1.76 mmol), tert-butyl carbamate (300 mg, 2.56 mmol), tris(dibenzylideneacetone)dipalladium (242 mg, 0.26 mmol), Xantphos (205 mg, 0.35 mmol) and cesium carbonate (1.7 g, 5.21 mmol) in 1,4-dioxane (10 mL) was stirred for 2 h at 100° C. The solids were filtered out. The solvent was removed under reduced pressure. The residue was purified by silica gel column chromatography eluting with PE/EA (5:1) to afford the title compound (800 mg, 84.93% yield) as a yellow solid. LCMS (ESI): [M+H]⁺=534.2.

Step 8: Methyl 2-((tert-butoxycarbonyl)amino)-4-(4-(2,2-difluoroethyl)piperazin-2-yl)benzoate. Under hydrogen, a mixture of benzyl 2-(3-((tert-butoxycarbonyl)amino)-4-(methoxycarbonyl)phenyl)-4-(2,2-difluoroethyl)piperazine-1-carboxylate (800 mg, 1.49 mmol) and 10% Pd/C (400 mg) in ethyl acetate (10 mL) was stirred for overnight at rt. The solids were filtered out. The solvent was removed under reduced pressure to afford the title compound (520 mg, 86.8% yield) as a yellow solid. LCMS (ESI) [M+H]⁺=400.2.

Step 9: tert-Butyl 4-((2-(3-((tert-butoxycarbonyl)amino)-4-(methoxycarbonyl)phenyl)-4-(2,2-difluoroethyl)piperazin-1-yl)methyl)-5-methoxy-7-methyl-1H-indole-1-carboxylate. A solution of methyl 2-((tert-butoxycarbonyl)amino)-4-(4-(2,2-difluoroethyl)piperazin-2-yl)benzoate (350 mg, 0.87 mmol), tert-butyl 4-formyl-5-methoxy-7-methyl-1H-indole-1-carboxylate (500 mg, 1.72 mmol) and acetic acid (0.1 mL) in 1,2-dichloroethane (3 mL) was stirred for 1 h at 70° C. Then sodium triacetoxyborohyride (350 mg, 1.65 mmol) was added. The resulting mixture was stirred for 7 h at 70° C. The solvent was removed under vacuum. The residue was purified by silica gel column chromatography eluting with PE/EA (5:1) to afford the title compound (350 mg, 59.37% yield) as a yellow solid. LCMS (ESI): [M+H]⁺=673.3.

Step 10:2-Amino-4-(4-(2,2-difluoroethyl)-1-((5-methoxy-7-methyl-1H-indol-4-yl)methyl)piperazin-2-yl)benzoic acid. A solution of tert-butyl 4-((2-(3-((tert-butoxycarbonyl)amino)-4-(methoxycarbonyl)phenyl)-4-(2,2-difluoroethyl)piperazin-1-yl)methyl)-5-methoxy-7-methyl-1H-indole-1-carboxylate (50 mg, 0.074 mmol) in 5 M potassium hydroxide (0.1 mL) and ethanol (1 mL) was stirred for 24 h at 100° C. The pH was adjust to 8 with 2 M HCl. The solvent was removed under vacuum. The residue was purified by Prep-HPLC to afford the title compound (9.7 mg, 28.4% yield) as a light pink solid. LCMS (ESI) [M+H]⁺=459.1, ¹H NMR (400 MHz, DMSO-d₆) δ 10.82 (s, 1H), 7.74 (d, J=8.1 Hz, 1H), 7.24 (t, J=2.8 Hz, 1H), 6.96-6.58 (m, 3H), 6.48 (dd, J=3.1, 1.9 Hz, 1H), 6.30-5.87 (m, 1H), 3.71 (s, 3H), 3.65 (d, J=11.8 Hz, 1H), 3.26-3.09 (m, 2H), 2.83-2.61 (m, 4H), 2.59-2.55 (m, 1H), 2.42 (s, 3H), 2.25 (t, J=10.7 Hz, 1H), 2.19-2.09 (m, 2H).

### Example 5. 2-Cyclopropyl-4-(4-(2,2-difluoroethyl)-1-((5-methoxy-7-methyl-1H-indol-4-yl)methyl)piperazin-2-yl)benzoic acid

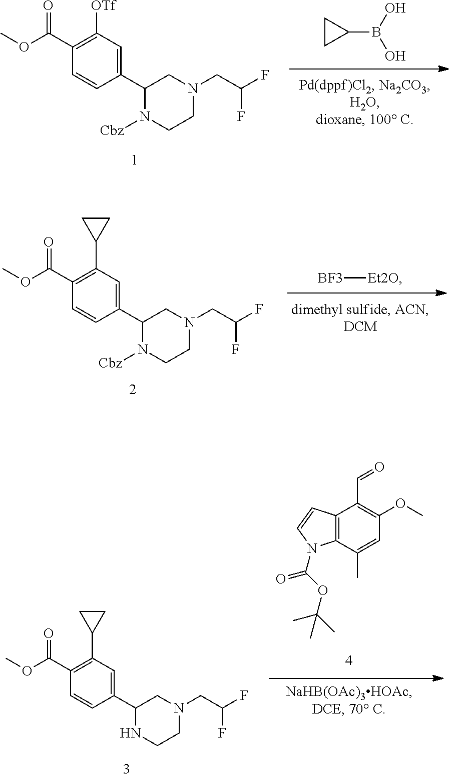

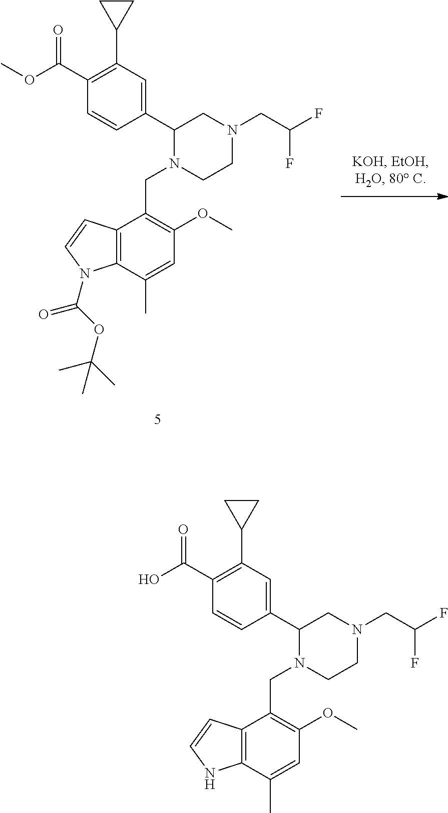 [SMILES: COC(=O)c1ccc(C2CN(CC(F)F)CCN2Cc2c(OC)cc(C)c3c2ccn3C(=O)OC(C)(C)C)cc1C1CC1 ; COc1cc(C)c2[nH]ccc2c1CN1CCN(CC(F)F)CC1c1ccc(C(=O)O)c(C2CC2)c1]

Step 1: Benzyl 2-(3-cyclopropyl-4-(methoxycarbonyl)phenyl)-4-(2,2-difluoroethyl)piperazine-1-carboxylate. Under nitrogen, to a solution of benzyl 4-(2,2-difluoroethyl)-2-(4-(methoxycarbonyl)-3-(((trifluoromethyl)sulfonyl)oxy)phenyl)piperazine-1-carboxylate (1 g, 1.76 mmol) and cyclopropylboronic acid (0.25 g, 2.91 mmol) in 1,4-dioxane (10 mL) and water (0.2 mL) was added sodium carbonate (0.60 g, 5.66 mmol) and [1,1′-bis(diphenylphosphino)ferrocene]dichloropalladium(II) (0.19 g, 0.26 mmol) and stirred for 4 h at 100° C. The solvent was removed. The residue was purified by silica gel column chromatography eluting with PE/EA (4:1) to afford the title compound (600 mg, 74.1% yield) as a yellow solid. LCMS (ESI): [M+H]⁺=459.2.

Step 2: Methyl 2-cyclopropyl-4-(4-(2,2-difluoroethyl)piperazin-2-yl)benzoate. To a stirred mixture of benzyl 2-(3-cyclopropyl-4-(methoxycarbonyl)phenyl)-4-(2,2-difluoroethyl)piperazine-1-carboxylate (500 mg, 1.09 mmol) in dichloromethane (5 mL) and acetonitrile (5 mL) was added dimethyl sulfide (0.5 mL) and boron trifluoride diethyl etherate (0.5 mL) at 0° C. The resulting mixture was stirred for additional 4 h at the same temperature. The reaction was quenched with saturated sodium carbonate solution (10 mL) and extracted with ethyl acetate (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with PE/EA (5:1) to afford the title compound (250 mg, 70.68%) as a yellow oil. LCMS (ESI): [M+H]⁺=325.2.

Step 3: tert-Butyl 4-((2-(3-cyclopropyl-4-(methoxycarbonyl)phenyl)-4-(2,2-difluoroethyl)piperazin-1-yl)methyl)-5-methoxy-7-methyl-1H-indole-1-carboxylate. A solution of methyl 2-cyclopropyl-4-(4-(2,2-difluoroethyl)piperazin-2-yl)benzoate (300 mg, 0.92 mmol), tert-butyl 4-formyl-5-methoxy-7-methylindole-1-carboxylate (540 mg, 1.86 mmol), sodium triacetoxyborohydride (390 mg, 1.84 mmol) and acetic acid (0.1 mL), in 1,2-dichloroethane (5 mL) was stirred for 6 h at 70° C. The resulting mixture was concentrated under reduced pressure. The residue was purified by silica gel column chromatography eluting with PE/EA (5:1) to afford the title compound (300 mg, 54.3% yield) as a yellow solid. LCMS (ESI): [M+H]⁺=598.3.

Step 4: 2-Cyclopropyl-4-(4-(2,2-difluoroethyl)-1-((5-methoxy-7-methyl-1H-indol-4-yl)methyl)piperazin-2-yl)benzoic acid. A solution of tert-Butyl 4-((2-(3-cyclopropyl-4-(methoxycarbonyl)phenyl)-4-(2,2-difluoroethyl)piperazin-1-yl)methyl)-5-methoxy-7-methyl-1H-indole-1-carboxylate (300 mg, 0.50 mmol) and potassium hydroxide (600 mg, 10.69 mmol) in EtOH (5 mL) and water (0.5 mL) was stirred for additional 2 h at 100° C. The pH was adjusted to 8 with 2 M HCl. The solvent was removed under vacuum. The residue was purified by reverse phase flash chromatography eluting with methanol/10 mM ammonium bicarbonate (10%-100%) to afford the compound (95.0 mg, 39.2% yield) as a white solid. LCMS (ESI) [M+H]⁺=484.2, ¹H NMR (300 MHz, DMSO-d₆) δ 10.83 (s, 1H), 7.70 (d, J=7.8 Hz, 1H), 7.37 (s, 1H), 7.24 (t, J=2.8 Hz, 1H), 7.08 (s, 1H), 6.64 (s, 1H), 6.43 (t, J=2.5 Hz, 1H), 6.36-5.81 (m, 1H), 3.69 (s, 3H), 3.53 (d, J=12.0 Hz, 1H), 3.31-3.08 (m, 3H), 2.91-2.51 (m, 6H), 2.40 (s, 3H), 2.34-2.08 (m, 3H), 0.95 (d, J=8.4 Hz, 2H), 0.72 (brs, 2H).

### Example 6. 8-(4-(2,2-Difluoroethyl)-1-((5-methoxy-7-methyl-1H-indol-4-yl)methyl)piperazin-2-yl)imidazo[1,2-a]pyridine-5-carboxylic acid

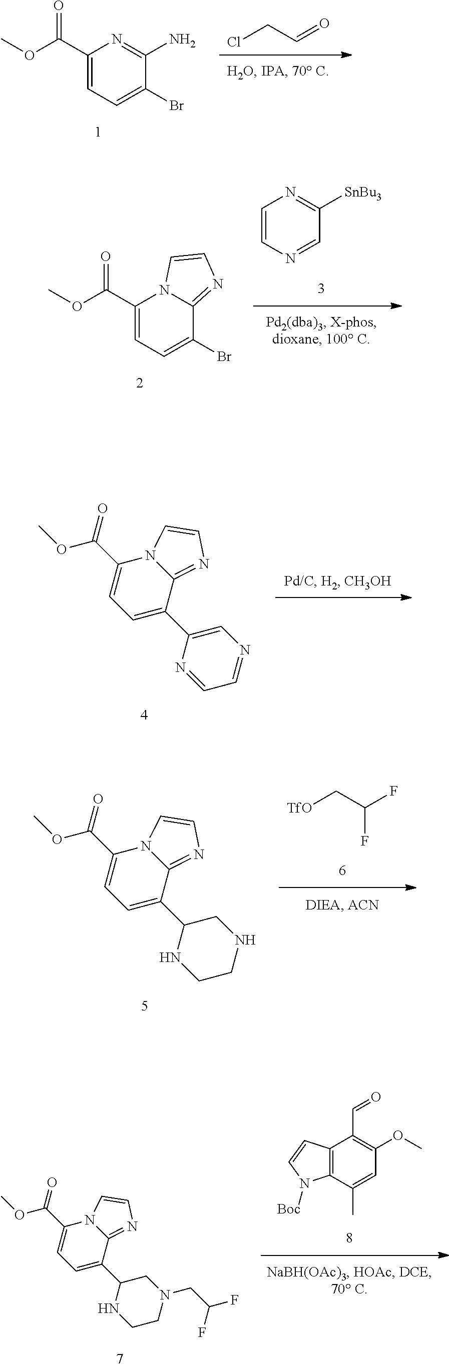

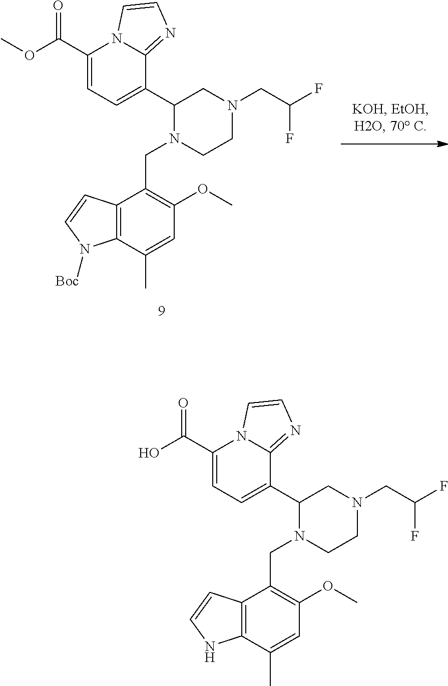 [SMILES: COC(=O)c1ccc(C2CN(CC(F)F)CCN2Cc2c(OC)cc(C)c3c2ccn3C(=O)OC(C)(C)C)c2nccn12 ; COc1cc(C)c2[nH]ccc2c1CN1CCN(CC(F)F)CC1c1ccc(C(=O)O)n2ccnc12]

Step 1: Methyl 8-bromoimidazo[1,2-a]pyridine-5-carboxylate. A mixture of methyl 6-amino-5-bromopyridine-2-carboxylate (2 g, 8.656 mmol) and chloroacetaldehyde (3 mL, 10 mmol) in propan-2-ol (30 mL, 0.017 mmol) was stirred for 3 h at 70° C. The mixture was allowed to cool down to room temperature. The precipitated solids were collected by filtration and washed with petroleum ether. This resulted in the desired isomer (2.2 g, 89.7% yield) as a white solid. LCMS (ESI) [M+H]⁺=254.9.

Step 2: Methyl 8-(pyrazin-2-yl)imidazo[1,2-a]pyridine-5-carboxylate. A mixture of methyl 8-bromoimidazo[1,2-a]pyridine-5-carboxylate (1.5 g, 5.88 mmol), 2-(tributylstannyl)pyrazine (4.34 g, 11.76 mmol), Pd₂(dba)₃ (1.08 g, 1.17 mmol) and XPhos (0.56 g, 1.17 mmol) in 1,4-dioxane (10 ml) was stirred for 2 h at 100° C. under nitrogen atmosphere. The solids were filtered out. The filtrate was concentrated by vacuum. The residue was purified by silica gel column chromatography, eluted with DCM/MeOH (9:1) to afford the title compound (507 mg, 22.0% yield) as a yellow solid. LCMS (ESI): [M+H]⁺=255.0.

Step 3: Methyl 8-(piperazin-2-yl)imidazo[1,2-a]pyridine-5-carboxylate. A mixture of methyl 8-(pyrazin-2-yl)imidazo[1,2-a]pyridine-5-carboxylate (480 mg, 1.88 mmol) and Pd/C (480 mg, 4.51 mmol) in MeOH (10 mL) was stirred for 3 h at 50° C. under hydrogen atmosphere. The solids were filtered out. The filtrate was concentrated by vacuum to afford the title compound (400 mg, 82.3% yield) as a yellow oil. LCMS (ESI): [M+H]⁺=261.1.

Step 4: Methyl 8-(4-(2,2-difluoroethyl)piperazin-2-yl)imidazo[1,2-a]pyridine-5-carboxylate. A mixture of methyl 8-(piperazin-2-yl)imidazo[1,2-a]pyridine-5-carboxylate (400 mg, 1.53 mmol), N,N-diisopropylethylamine (595 mg, 4.61 mmol) and 2,2-difluoroethyl trifluoromethanesulfonate (493 mg, 2.30 mmol) in acetonitrile (5 mL) was stirred for 2 h at room temperature. The resulting mixture was quenched with water and extracted with ethyl acetate. The solvent was removed under reduced pressure. The residue was purified by silica gel column chromatography, eluted with CHCl₃/MeOH (9:1) to afford the title compound (90 mg, 18.1% yield) as a yellow oil. LCMS (ESI): [M+H]⁺=325.1.

Step 5: Methyl 8-(1-((1-(tert-butoxycarbonyl)-5-methoxy-7-methyl-1H-indol-4-yl)methyl)-4-(2,2-difluoroethyl)piperazin-2-yl)imidazo[1,2-a]pyridine-5-carboxylate. A mixture of methyl 8-(4-(2,2-difluoroethyl)piperazin-2-yl)imidazo[1,2-a]pyridine-5-carboxylate (75 mg, 0.23 mmol), tert-butyl 4-formyl-5-methoxy-7-methylindole-1-carboxylate (133 mg, 0.46 mmol), sodium triacetoxyborohydride (146 mg, 0.68 mmol) and HOAc (28 mg, 0.46 mmol) in dichloroethane (5 mL) was stirred for 5 h at 70° C. The resulting mixture was concentrated under reduced pressure. The residue was purified by reversed-phase flash chromatography with MeCN/10 mmol/L NH₄HCO₃ in water to afford the title compound (133 mg, 77.0% yield) as a yellow solid. LCMS (ESI): [M+H]⁺=598.2.

Step 6: 8-(4-(2,2-Difluoroethyl)-1-((5-methoxy-7-methyl-1H-indol-4-yl)methyl)piperazin-2-yl)imidazo[1,2-a]pyridine-5-carboxylic acid. A mixture of Methyl 8-(1-((1-(tert-butoxycarbonyl)-5-methoxy-7-methyl-1H-indol-4-yl)methyl)-4-(2,2-difluoroethyl)piperazin-2-yl)imidazo[1,2-a]pyridine-5-carboxylate (127 mg, 0.21 mmol), Potassium hydroxide (100 mg, 1.78 mmol) in water (1 mL) in Ethanol (5 mL) was stirred for 1 h at 70° C. The mixture was acidified to pH=7 with sat. citric acid solution. The solvent was removed under vacuum. The residue was purified by reversed-phase flash chromatography and Prep-HPLC to afford the title compound (22.3 mg, 20.7% yield) as a white solid. LCMS (ESI): [M+H]⁺=484.2 ¹H NMR (300 MHz, Methanol-d₄) δ 9.01 (d, J=1.5 Hz, 1H), 7.69 (d, J=1.5 Hz, 1H), 7.63-7.55 (m, 2H), 7.26 (d, J=3.2 Hz, 1H), 6.78-6.49 (m, 1H), 6.34 (d, J=3.2 Hz, 1H), 6.00 (tt, J=55.8, 4.2 Hz, 1H), 4.88-4.73 (m, 1H), 4.37-4.08 (m, 2H), 3.73 (s, 3H), 3.42 (d, J=11.8 Hz, 1H), 3.26-3.04 (m, 4H), 2.96-2.70 (m, 3H), 2.46-2.35 (m, 3H).

### Example 7. 6-(4-(2,2-Difluoroethyl)-1-((5-methoxy-7-methyl-1H-indol-4-yl)methyl)piperazin-2-yl)-2-hydroxynicotinamide

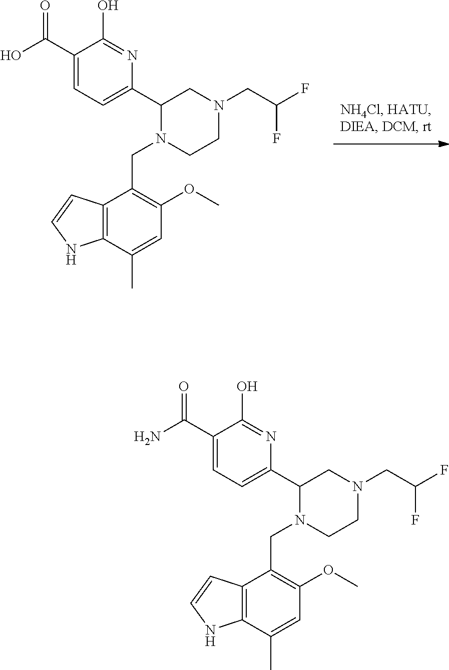 [SMILES: COc1cc(C)c2[nH]ccc2c1CN1CCN(CC(F)F)CC1c1ccc(C(=O)O)c(O)n1 ; COc1cc(C)c2[nH]ccc2c1CN1CCN(CC(F)F)CC1c1ccc(C(N)=O)c(O)n1]

A solution of 6-(4-(2,2-difluoroethyl)-1-((5-methoxy-7-methyl-1H-indol-4-yl)methyl)piperazin-2-yl)-2-hydroxynicotinic acid (75 mg, 0.16 mmol), ammonium chloride (87 mg, 1.62 mmol) and N,N-diisopropylethylamine (180 mg, 1.39 mmol) in dichloromethane (1 mL) was added HATU (93 mg, 0.24 mmol) at 0° C. The resulting mixture was stirred for additional 5 h at room temperature. The crude product was purified by reverse phase flash and Prep-HPLC to afford the title compound (22 mg, 28.2% yield) as a light-yellow solid. LCMS (ESI): [M+H]⁺=460.2. ¹H NMR (300 MHz, Methanol-d₄) δ 8.41 (d, J=7.4 Hz, 1H), 7.23 (d, J=3.1 Hz, 1H), 6.72 (s, 1H), 6.57 (d, J=7.5 Hz, 1H), 6.49 (d, J=3.1 Hz, 1H), 6.16-5.74 (m, 1H), 3.90 (s, 3H), 3.84 (d, J=12.0 Hz, 1H), 3.61-3.45 (m, 2H), 3.00-2.91 (m, 1H), 2.84-2.66 (m, 4H), 2.55-2.29 (m, 6H).

### Example 8. (S)-2-Amino-4-(4-(2,2-difluoroethyl)-1-((5-(difluoromethoxy)-7-methyl-1H-indol-4-yl)methyl)piperazin-2-yl)benzoic acid & (R)-2-amino-4-(4-(2,2-difluoroethyl)-1-((5-(difluoromethoxy)-7-methyl-1H-indol-4-yl)methyl)piperazin-2-yl)benzoic acid

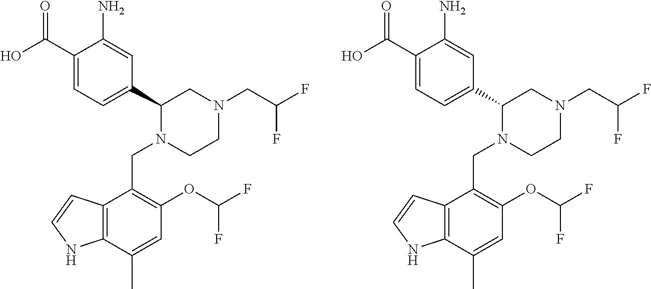 [SMILES: Cc1cc(OC(F)F)c(CN2CCN(CC(F)F)C[C@@H]2c2ccc(C(=O)O)c(N)c2)c2cc[nH]c12 ; Cc1cc(OC(F)F)c(CN2CCN(CC(F)F)C[C@H]2c2ccc(C(=O)O)c(N)c2)c2cc[nH]c12]

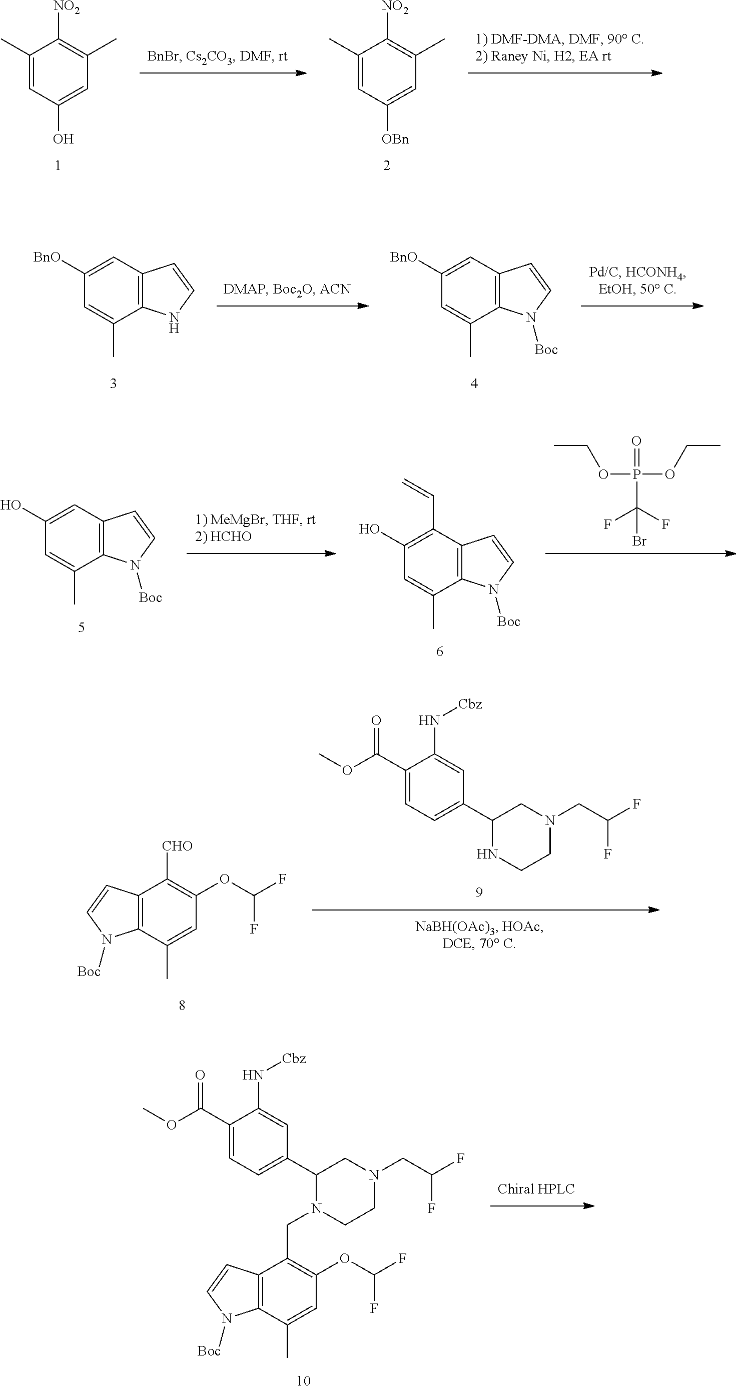

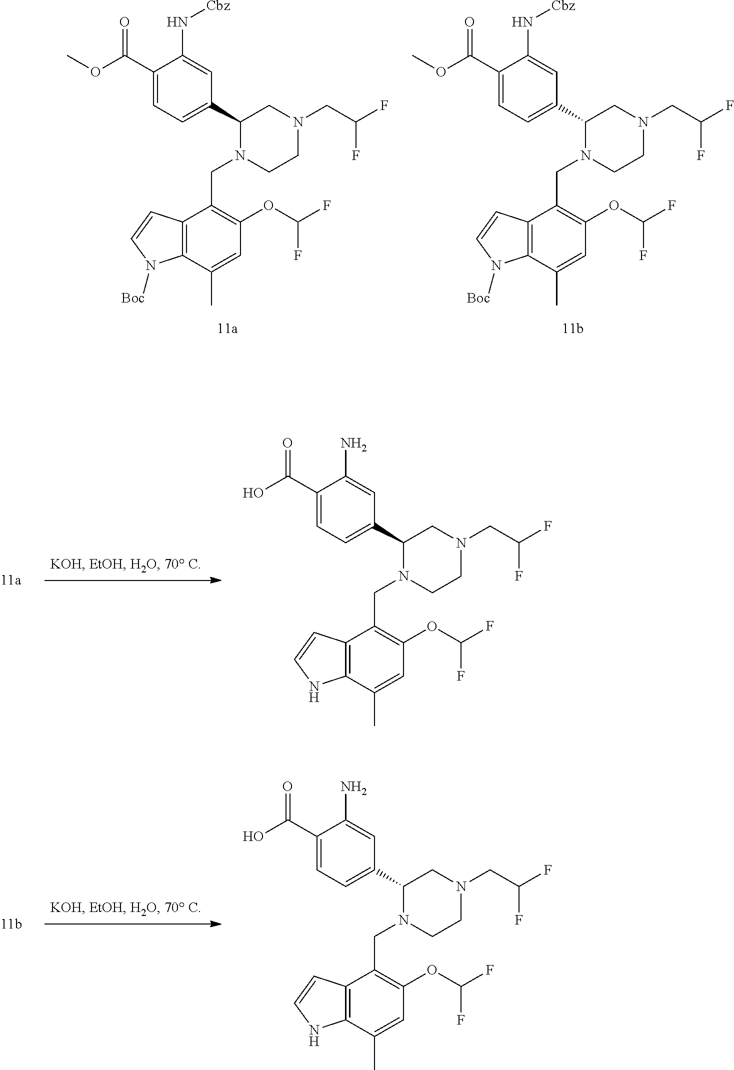 [SMILES: COC(=O)c1ccc([C@H]2CN(CC(F)F)CCN2Cc2c(OC(F)F)cc(C)c3c2ccn3C(=O)OC(C)(C)C)cc1NC(=O)OCc1ccccc1 ; COC(=O)c1ccc([C@@H]2CN(CC(F)F)CCN2Cc2c(OC(F)F)cc(C)c3c2ccn3C(=O)OC(C)(C)C)cc1NC(=O)OCc1ccccc1 ; Cc1cc(OC(F)F)c(CN2CCN(CC(F)F)C[C@@H]2c2ccc(C(=O)O)c(N)c2)c2cc[nH]c12 ; Cc1cc(OC(F)F)c(CN2CCN(CC(F)F)C[C@H]2c2ccc(C(=O)O)c(N)c2)c2cc[nH]c12]

Step 1: 5-(Benzyloxy)-1,3-dimethyl-2-nitrobenzene. To a stirred solution of 3,5-dimethyl-4-nitrophenol (14 g, 83.75 mmol) and Cesium carbonate (32 g, 98.82 mmol) in DMF (140 mL) was added (bromomethyl)benzene (14 g, 82.07 mmol) by dropwise and stirred for 24 h at room temperature. The reaction was quenched with water and extracted with ethyl acetate. The organic layer was washed with brine. The solvent was removed to afford the title compound (21 g, 97.4% yield) as a brown yellow oil and as crude. LCMS (ESI): [M+H]⁺=258.2.

Step 2: 5-(Benzyloxy)-7-methyl-1H-indole. A solution of 5-(benzyloxy)-1,3-dimethyl-2-nitrobenzene (10 g, 38.86 mmol) and pyrrolidine (8 g, 117.37 mmol), DMF-DMA (10 mL) in DMF (100 mL) was stirred for overnight at 90° C. The DMF-DMA was removed under vacuum. The residue was dissolved in ethyl acetate (300 mL). Then Raney Ni (14.8 g) was added. The mixture was stirred for 14 h under hydrogen. The solids were filtered out. The filtrate was washed with brine. The solvent was removed. The residue was purified by silica gel column chromatography, eluted with petroleum ether/ethyl acetate (5:1) to afford the title compound (5 g, 44.6% yield) as a Brown yellow oil. LCMS (ESI): [M+H]⁺=237.1.

### Step 3: tert-Butyl 5-(benzyloxy)-7-methylindole-1-carboxylate

A solution of 5-(benzyloxy)-7-methyl-1H-indole (4 g, 19.80 mmol) and Di-tert-butyl dicarbonate (4 g, 19.80 mmol), DMAP (460 mg, 3.76 mmol) in ACN (100 mL) was stirred for 1 h at room temperature under nitrogen atmosphere. The reaction was quenched with water and extracted with ethyl acetate. The organic layer was washed with brine. The solvent was removed under vacuum. The residue was purified by silica gel column chromatography, eluted with petroleum ether/ethyl acetate (5:1) to afford the title compound (5 g, 70.3% yield) as a Brown yellow oil. LCMS (ESI): [M+H]⁺=337.4.

Step 4: tert-Butyl 5-hydroxy-7-methylindole-1-carboxylate. A mixture of tert-butyl 5-(benzyloxy)-7-methylindole-1-carboxylate (5 g, 13.92 mmol) and 10% Pd/C (652 mg, 6.12 mmol), ammonium formate (465 mg, 7.38 mmol) in Ethanol (20 mL) was stirred for 5 h at 50° C. The reaction was quenched with water and extracted with ethyl acetate. The organic layer was washed with brine. The solvent was removed under vacuum. The residue was purified by silica gel column chromatography, eluted with petroleum ether/ethyl acetate (5:1) to afford the title compound (3 g, 84.1% yield) as a brown yellow oil. LCMS (ESI): [M+H]⁺=248.2.

Step 5: tert-Butyl 4-formyl-5-hydroxy-7-methylindole-1-carboxylate. A solution of tert-butyl 5-hydroxy-7-methylindole-1-carboxylate (3 g, 11.72 mmol), Magnesium monochloride (3.3 g, 35.29 mmol) and triethylamine (5 mL) in THF (65 mL) was added Paraformaldehyde (1450 mg, 48.31 mmol) in portions and stirred for 1 h at 70° C. The solvent was removed under vacuum. The residue was purified by silica gel column chromatography, eluted with petroleum ether/ethyl acetate (5:1) to afford the title compound (3 g, 92.9% yield) as a green powder. LCMS (ESI): [M+H]⁺=276.3.

Step 6: tert-Butyl 5-(difluoromethoxy)-4-formyl-7-methylindole-1-carboxylate. A mixture of tert-butyl 4-formyl-5-hydroxy-7-methylindole-1-carboxylate (600 mg, 2.17 mmol), diethyl bromodifluoromethylphosphonate (2327 mg, 8.71 mmol) and Potassium hydroxide (2445 mg, 43.58 mmol) in ACN (6 mL) and water (6 mL) was stirred for 3 h at room temperature. The reaction was quenched with water and extracted with ethyl acetate. The organic layer was washed with brine. The solvent was removed under vacuum. The residue was purified by silica gel column chromatography, eluted with petroleum ether/ethyl acetate (5:1) to afford the title compound (510 mg, 54.6% yield) as a light yellow solid. LCMS (ESI): [M+H]⁺=325.3.

Step 7: tert-Butyl 4-((2-(3-(((benzyloxy)carbonyl)amino)-4-(methoxycarbonyl)phenyl)-4-(2,2-difluoroethyl)piperazin-1-yl)methyl)-5-(difluoromethoxy)-7-methylindole-1-carboxylate. A mixture of methyl 2-(((benzyloxy)carbonyl)amino)-4-(4-(2,2-difluoroethyl)piperazin-2-yl)benzoate (200 mg, 0.46 mmol) and tert-butyl 5-(difluoromethoxy)-4-formyl-7-methylindole-1-carboxylate (210 mg, 0.64 mmol), Sodium triacetoxyborohydride (195 mg, 0.92 mmol), HOAc (27 mg, 0.46 mmol) in dichloroethane (0.5 mL) was stirred for 2 h at 70° C. The solvent was removed under vacuum. The residue was purified by silica gel column chromatography, eluted with petroleum ether/ethyl acetate (5:1) to afford the title compound (190 mg, 54.7% yield) as a light yellow solid. LCMS (ESI): [M+H]⁺=743.3.

Step 8: tert-Butyl(S)-4-((2-(3-(((benzyloxy)carbonyl)amino)-4-(methoxycarbonyl)phenyl)-4-(2,2-difluoroethyl)piperazin-1-yl)methyl)-5-(difluoromethoxy)-7-methyl-1H-indole-1-carboxylate (isomer 1, slower peak)

tert-Butyl (R)-4-((2-(3-(((benzyloxy)carbonyl)amino)-4-(methoxycarbonyl)phenyl)-4-(2,2-difluoroethyl)piperazin-1-yl)methyl)-5-(difluoromethoxy)-7-methyl-1H-indole-1-carboxylate (isomer 2, faster peak)

tert-Butyl 4-((2-(3-(((benzyloxy)carbonyl)amino)-4-(methoxycarbonyl)phenyl)-4-(2,2-difluoroethyl)piperazin-1-yl)methyl)-5-(difluoromethoxy)-7-methylindole-1-carboxylate (190 mg) was applied for further purification by Chiral HPLC with the following conditions:

Column: CHIRALPAK IG-3, 4.6*50 mm, 3 μm; Mobile Phase A: Hex (0.1% DEA): Ethanol=90:10; Flow rate: 1 mL/min; Gradient: isocratic; Injection Volume: 5 ul mL. Number of Runs: 6.

tert-Butyl(S)-4-((2-(3-(((benzyloxy)carbonyl)amino)-4-(methoxycarbonyl)phenyl)-4-(2,2-difluoroethyl)piperazin-1-yl)methyl)-5-(difluoromethoxy)-7-methyl-1H-indole-1-carboxylate (isomer 1, faster peak) (80 mg, 42.1% yield). LCMS (ESI): [M+H]⁺=743.3, tR=1.58 min (CHIRALPAK IG-3, 4.6*50 mm 3 μm), Hex (0.1% DEA):IPA-90:10, 1.0 mL/min, 254 nm).

tert-Butyl(S)-4-((2-(3-(((benzyloxy)carbonyl)amino)-4-(methoxycarbonyl)phenyl)-4-(2,2-difluoroethyl)piperazin-1-yl)methyl)-5-(difluoromethoxy)-7-methyl-1H-indole-1-carboxylate (isomer 1, slower peak) (88 mg, 46.3% yield). LCMS (ESI): [M+H]⁺=743.3, tR=2.19 min (CHIRALPAK IG-3, 4.6*50 mm 3 μm), Hex (0.1% DEA):IPA=90:10, 1.0 mL/min, 254 nm).

Step 9: (S)-2-Amino-4-(4-(2,2-difluoroethyl)-1-((5-(difluoromethoxy)-7-methyl-1H-indol-4-yl)methyl)piperazin-2-yl)benzoic acid & (R)-2-amino-4-(4-(2,2-difluoroethyl)-1-((5-(difluoromethoxy)-7-methyl-1H-indol-4-yl)methyl)piperazin-2-yl)benzoic acid. A mixture of tert-butyl 4-(((2S)-2-(3-(((benzyloxy)carbonyl)amino)-4-(methoxycarbonyl)phenyl)-4-(2,2-difluoroethyl)piperazin-1-yl)methyl)-5-(difluoromethoxy)-7-methylindole-1-carboxylate (80 mg, 0.11 mmol) and Potassium hydroxide (75 mg, 1.35 mmol) in ethanol (1 mL) and water (0.2 mL) was stirred for 2 h at 70° C. The mixture was allowed to cool down to room temperature and was neutralized to pH=6 with sat. Citric acid. The solvent was removed. The residue was purified by reversed-phase flash chromatography with the following conditions: column, C18 silica gel; mobile phase, acetonitrile in Water (10 mmol/L NH₄HCO₃), 5% to 50% gradient in 20 min; detector, UV 254 nm. This resulted in (S)-2-Amino-4-(4-(2,2-difluoroethyl)-1-((5-(difluoromethoxy)-7-methyl-1H-indol-4-yl)methyl)piperazin-2-yl)benzoic acid (32.7 mg, 49.0% yield) as a white solid. LCMS (ESI): [M+H]⁺=495.2. ¹H NMR (300 MHz, Methanol-d₄) δ 7.88 (d, J=8.2 Hz, 1H), 7.30 (d, J=3.1 Hz, 1H), 6.94 (s, 1H), 6.88-6.36 (m, 4H), 6.19-5.80 (m, 1H), 3.92 (d, J=12.2 Hz, 1H), 3.39 (s, 2H), 2.96-2.85 (m, 2H), 2.81-2.65 (m, 3H), 2.57-2.45 (m, 4H), 2.44-2.31 (m, 2H).

A mixture of tert-butyl 4-(((2R)-2-(3-(((benzyloxy)carbonyl)amino)-4-(methoxycarbonyl)phenyl)-4-(2,2-difluoroethyl)piperazin-1-yl)methyl)-5-(difluoromethoxy)-7-methylindole-1-carboxylate (88 mg, 0.11 mmol) and Potassium hydroxide (66 mg, 1.18 mmol) in Ethanol (1 mL) and water (0.2 mL) was stirred for 2 h at 70° C. The mixture was allowed to cool down to room temperature and was neutralized to pH=6 with sat. Citric acid. The residue was purified by reversed-phase flash chromatography with the following conditions: column, C18 silica gel; mobile phase, acetonitrile in Water (10 mmol/L NH₄HCO₃), 5% to 50% gradient in 20 min; detector, UV 254 nm. This resulted in (R)-2-Amino-4-(4-(2,2-difluoroethyl)-1-((5-(difluoromethoxy)-7-methyl-1H-indol-4-yl)methyl)piperazin-2-yl)benzoic acid (27 mg, 46.1% yield) as a white solid. LCMS (ESI): [M+H]⁺=495.2. ¹H NMR (300 MHz, Methanol-d₄) δ 7.88 (d, J=8.2 Hz, 1H), 7.30 (d, J=3.1 Hz, 1H), 6.94 (s, 1H), 6.88-6.36 (m, 4H), 6.19-5.80 (m, 1H), 3.92 (d, J=12.2 Hz, 1H), 3.39 (s, 2H), 2.96-2.85 (m, 2H), 2.81-2.65 (m, 3H), 2.57-2.45 (m, 4H), 2.44-2.31 (m, 2H).

### Example 9. (R)-6-(4-(2,2-Difluoroethyl)-1-((5-methoxy-7-methyl-1H-indol-4-yl)methyl)piperazin-2-yl)-2-hydroxynicotinic acid & (S)-6-(4-(2,2-difluoroethyl)-1-((5-methoxy-7-methyl-1H-indol-4-yl)methyl)piperazin-2-yl)-2-hydroxynicotinic acid

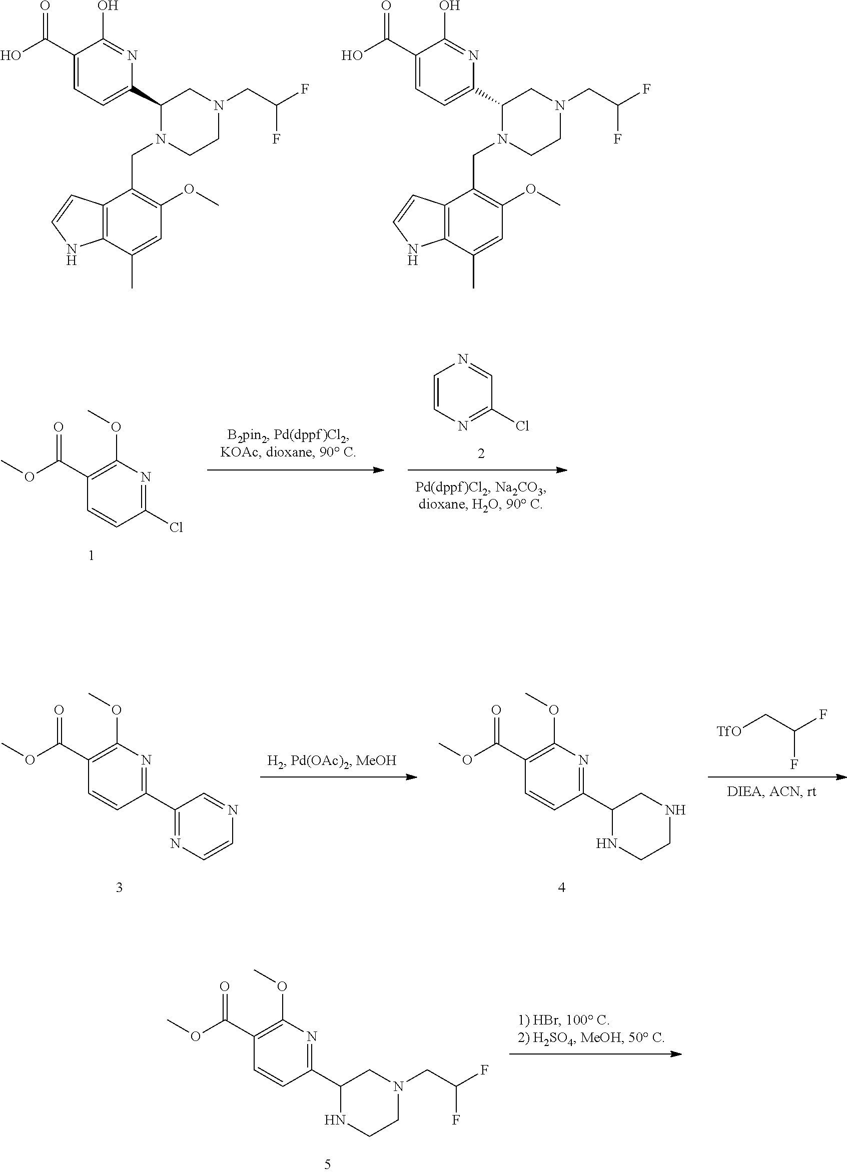

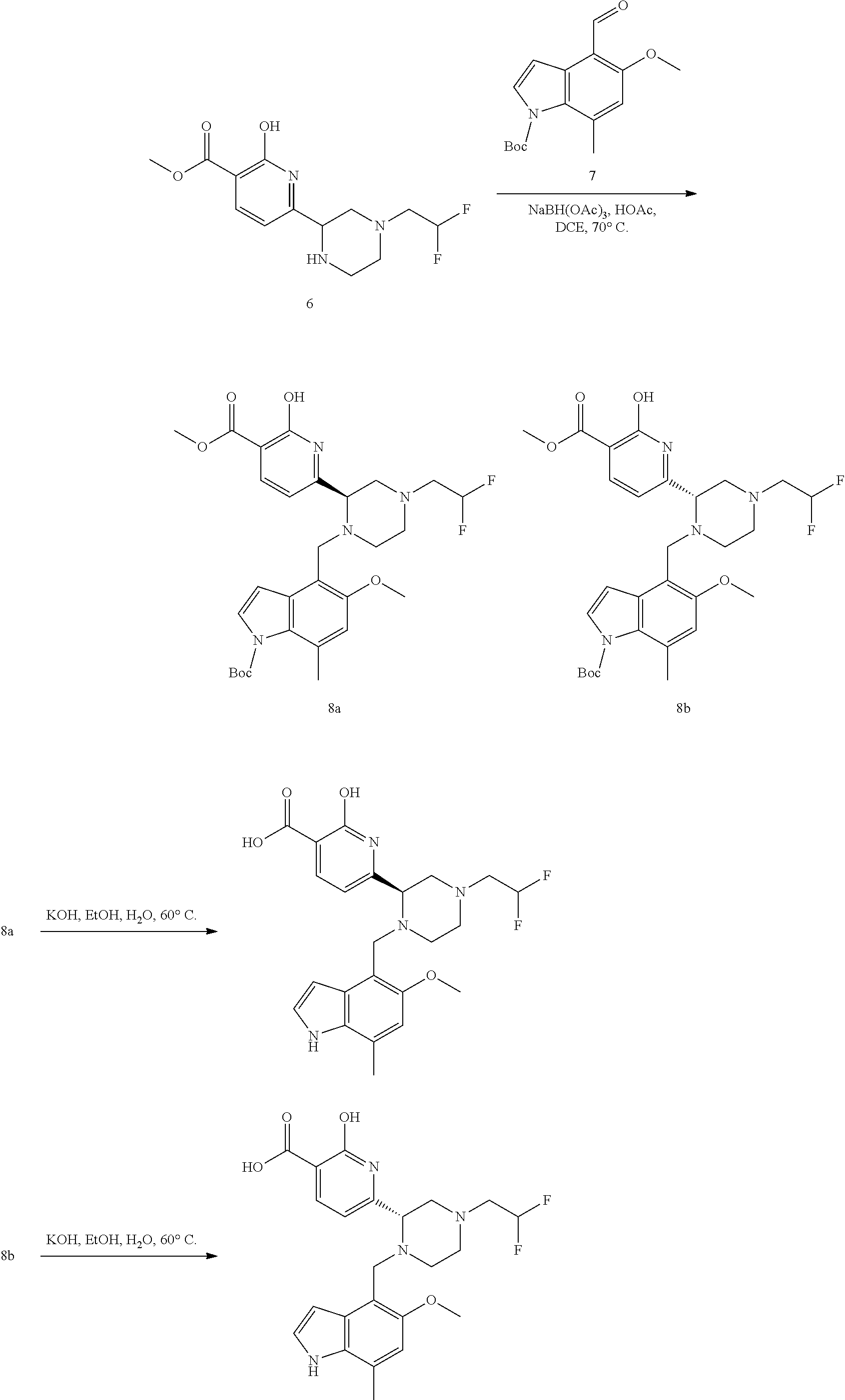 [SMILES: COC(=O)c1ccc(C2CN(CC(F)F)CCN2)nc1O ; COc1cc(C)c2c(ccn2C(=O)OC(C)(C)C)c1C=O ; COC(=O)c1ccc([C@H]2CN(CC(F)F)CCN2Cc2c(OC)cc(C)c3c2ccn3C(=O)OC(C)(C)C)nc1O ; COC(=O)c1ccc([C@@H]2CN(CC(F)F)CCN2Cc2c(OC)cc(C)c3c2ccn3C(=O)OC(C)(C)C)nc1O ; COc1cc(C)c2[nH]ccc2c1CN1CCN(CC(F)F)C[C@@H]1c1ccc(C(=O)O)c(O)n1 ; COc1cc(C)c2[nH]ccc2c1CN1CCN(CC(F)F)C[C@H]1c1ccc(C(=O)O)c(O)n1]

Step 1: Methyl 2-methoxy-6-(pyrazin-2-yl)pyridine-3-carboxylate. A mixture of methyl 6-chloro-2-methoxypyridine-3-carboxylate (3 g, 14.88 mmol), bis(pinacolato)diboron (5.7 g, 22.32 mmol), KOAc (4.4 g, 44.64 mmol) and Pd(dppf)Cl₂ (1.6 g, 2.23 mmol) in dioxane (30 mL) was stirred for 4 h at 90° C. under nitrogen atmosphere. To the above mixture was added Na₂CO₃ (4.5 g, 42.46 mmol), Pd(dppf)Cl₂ (1.0 g, 1.37 mmol), 2-chloropyrazine (3.3 g, 28.81 mmol) and water (6 mL). The resulting mixture was stirred for overnight at 90° C. under nitrogen atmosphere. The solids were filtered out. The filtrate was concentrated under reduced pressure. The filtrate was concentrated under reduced pressure. The residue was purified by silica gel column chromatography, eluted with ethyl acetate/petroleum ether (0-30%) to afford the title compound (3.4 g, 81.1% yield) as a white solid. LCMS (ESI): [M+H]⁺=245.8.

Step 2: Methyl 2-methoxy-6-(piperazin-2-yl)pyridine-3-carboxylate. A solution of methyl 2-methoxy-6-(pyrazin-2-yl)pyridine-3-carboxylate (3.4 g, 13.86 mmol) and Pd(OAc)₂ (0.3 g, 1.34 mmol) in MeOH (30 mL) was stirred for overnight at room temperature under hydrogen atmosphere. The solids were filtered out. The filtrate was concentrated under reduced pressure to afford the title compound (2.7 g, 64.2% yield) as a yellow solid. LCMS (ESI): [M+H]⁺=252.0.

Step 3: Methyl 6-(4-(2,2-difluoroethyl)piperazin-2-yl)-2-methoxypyridine-3-carboxylate. To a stirred mixture of methyl 2-methoxy-6-(piperazin-2-yl)pyridine-3-carboxylate (2.7 g, 10.74 mmol) and N,N-diisopropylethylamine (4.2 g, 32.49 mmol) in acetonitrile (50 mL) was added 2,2-difluoroethyl trifluoromethanesulfonate (1.1 g, 5.13 mmol) dropwise at 0° C. The resulting mixture was stirred for additional 2 h at room temperature. The resulting mixture was concentrated under reduced pressure. The residue was purified by reverse phase flash with the following conditions (acetonitrile/10 mM NH₄HCO₃ in water) to afford the title compound (1.1 g, 32.5% yield) as a yellow solid. LCMS (ESI): [M+H]⁺=316.2.

Step 4: Methyl 6-(4-(2,2-difluoroethyl)piperazin-2-yl)-2-hydroxynicotinate. A solution of methyl 6-(4-(2,2-difluoroethyl)piperazin-2-yl)-2-methoxypyridine-3-carboxylate (1 g, 3.17 mmol) in 48% HBr in water (2 mL) was stirred for overnight at 100° C. The solvent was removed under vacuum. The residue was dissolved in methanol (6 mL) and was added H₂SO₄ (0.05 mL). The resulting solution was stirred for overnight at 50° C. The mixture was neutralized to pH=7 with N,N-diisopropylethylamine. The crude product was purified by reverse phase flash with the following conditions (ACN/10 mM NH₄HCO₃ in water) to afford the title compound (579 mg, 46.45% yield) as a light yellow solid. LCMS (ESI): [M+H]⁺=302.2.

Step 5: tert-butyl (R)-4-((4-(2,2-difluoroethyl)-2-(6-hydroxy-5-(methoxycarbonyl)pyridin-2-yl)piperazin-1-yl)methyl)-5-methoxy-7-methyl-1H-indole-1-carboxylate & tert-butyl(S)-4-((4-(2,2-difluoroethyl)-2-(6-hydroxy-5-(methoxycarbonyl)pyridin-2-yl)piperazin-1-yl)methyl)-5-methoxy-7-methyl-1H-indole-1-carboxylate. A solution of Methyl 6-(4-(2,2-difluoroethyl)piperazin-2-yl)-2-hydroxynicotinate (560 mg, 1.85 mmol), HOAc (335 mg, 5.57 mmol) and tert-butyl 4-formyl-5-methoxy-7-methylindole-1-carboxylate (645 mg, 2.23 mmol) in DCE (5.6 mL) was stirred for 2 h at 70° C. To the above mixture was added sodium triacetoxyborohydride (788 mg, 3.72 mmol) at room temperature. The resulting mixture was stirred for overnight at 70° C. The solvent was removed under vacuum. The residue was purified by reverse phase flash with the following conditions (ACN/10 mM NH₄HCO₃ in water) and Chiral-HPLC to afford the title compounds.

tert-Butyl (R)-4-((4-(2,2-difluoroethyl)-2-(6-hydroxy-5-(methoxycarbonyl)pyridin-2-yl)piperazin-1-yl)methyl)-5-methoxy-7-methyl-1H-indole-1-carboxylate (89 mg, 8.30% yield, faster peak, isomer 1) as a yellow solid. LCMS (ESI): [M+H]⁺=575.3, tR=2.39 min (CHIRAL Cellulose-SB, 0.46*5 cm, 3 um, Hex (0.1% DEA):IPA=50:50, 1.0 mL/min, 254 nm).

tert-Butyl (R)-4-((4-(2,2-difluoroethyl)-2-(6-hydroxy-5-(methoxycarbonyl)pyridin-2-yl)piperazin-1-yl)methyl)-5-methoxy-7-methyl-1H-indole-1-carboxylate (96 mg, 8.90% yield, slower peak, isomer 2) as a yellow solid. LCMS (ESI): [M+H]⁺=575.3, tR=2.86 min (CHIRAL Cellulose-SB, 0.46*5 cm, 3 um, Hex (0.1% DEA):IPA=50:50, 1.0 mL/min, 254 nm).

Step 6: (R)-6-(4-(2,2-Difluoroethyl)-1-((5-methoxy-7-methyl-1H-indol-4-yl)methyl)piperazin-2-yl)-2-hydroxynicotinic acid & (S)-6-(4-(2,2-difluoroethyl)-1-((5-methoxy-7-methyl-1H-indol-4-yl)methyl)piperazin-2-yl)-2-hydroxynicotinic acid. A solution of tert-Butyl (R)-4-((4-(2,2-difluoroethyl)-2-(6-hydroxy-5-(methoxycarbonyl)pyridin-2-yl)piperazin-1-yl)methyl)-5-methoxy-7-methyl-1H-indole-1-carboxylate (89 mg, 0.16 mmol, faster peak, isomer 1) and Potassium hydroxide (87 mg, 1.55 mmol) in water (1 mL) and ethanol (2 mL) was stirred for overnight at 60° C. The mixture was acidified to pH=6 with sat. citric acid. The solvent was removed under vacuum. The residue was purified by Prep-HPLC to afford the title compound (27 mg, 38.3% yield) as a light-yellow solid. LCMS (ESI): [M+H]⁺=461.2. ¹H NMR (300 MHz, Methanol-d₄) δ 8.30 (d, J=7.5 Hz, 1H), 7.29 (d, J=3.1 Hz, 1H), 6.87 (d, J=7.5 Hz, 1H), 6.73 (s, 1H), 6.50 (d, J=3.2 Hz, 1H), 6.17-5.74 (m, 1H), 4.23-4.08 (m, 2H), 4.00 (d, J=12.4 Hz, 1H), 3.87 (s, 3H), 3.19-2.55 (m, 8H), 2.48 (s, 3H).

A solution of tert-Butyl (S)-4-((4-(2,2-difluoroethyl)-2-(6-hydroxy-5-(methoxycarbonyl)pyridin-2-yl)piperazin-1-yl)methyl)-5-methoxy-7-methyl-1H-indole-1-carboxylate (96 mg, 0.17 mmol, slower peak, isomer 2) and Potassium hydroxide (94 mg, 1.68 mmol) in water (1 mL) and Ethanol (2 mL) was stirred for overnight at 60° C. The mixture was acidified to pH=6 with sat. citric acid. The solvent was removed under vacuum and the residue was purified by Prep-HPLC to afford the title compound (23 mg, 29.76% yield) as a light-yellow solid. LCMS (ESI): [M+H]⁺=8461.1. ¹H NMR (300 MHz, Methanol-d₄) δ 8.29 (d, J=7.5 Hz, 1H), 7.28 (d, J=3.1 Hz, 1H), 6.88 (d, J=7.5 Hz, 1H), 6.72 (s, 1H), 6.50 (d, J=3.1 Hz, 1H), 6.16-5.74 (m, 1H), 4.12 (d, J=12.1 Hz, 2H), 4.01-3.91 (m, 1H), 3.87 (s, 3H), 3.17-2.53 (m, 8H), 2.48 (s, 3H).

### Example 10. (S)-4-(4-(2,2-Difluoroethyl)-1-((5-methoxy-7-methyl-1H-indol-4-yl)methyl)piperazin-2-yl)-2-(oxetan-3-yloxy)benzoic acid & (R)-4-(4-(2,2-difluoroethyl)-1-((5-methoxy-7-methyl-1H-indol-4-yl)methyl)piperazin-2-yl)-2-(oxetan-3-yloxy)benzoic acid

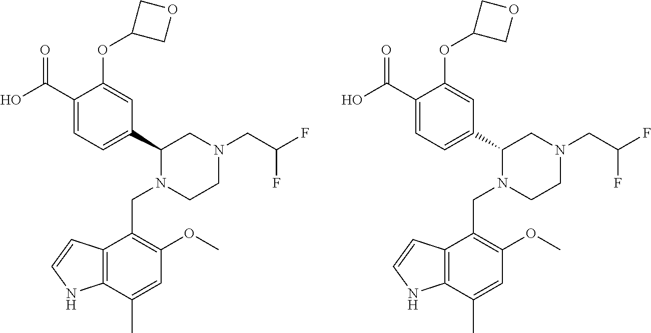 [SMILES: COc1cc(C)c2[nH]ccc2c1CN1CCN(CC(F)F)C[C@@H]1c1ccc(C(=O)O)c(OC2COC2)c1 ; COc1cc(C)c2[nH]ccc2c1CN1CCN(CC(F)F)C[C@H]1c1ccc(C(=O)O)c(OC2COC2)c1]

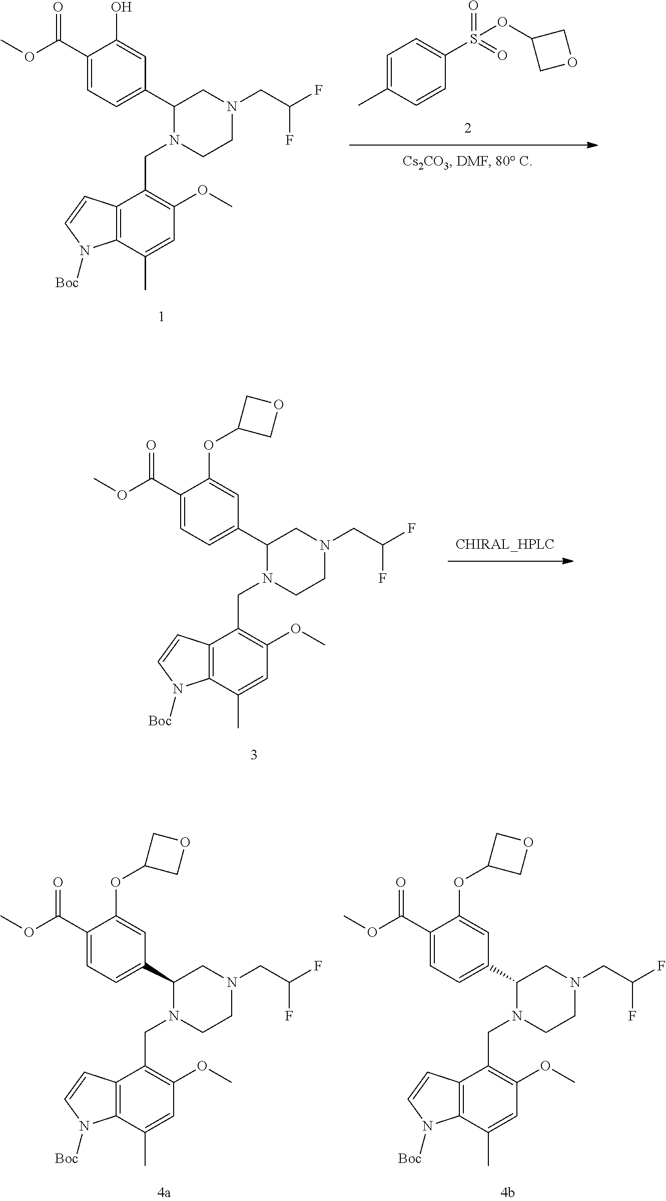

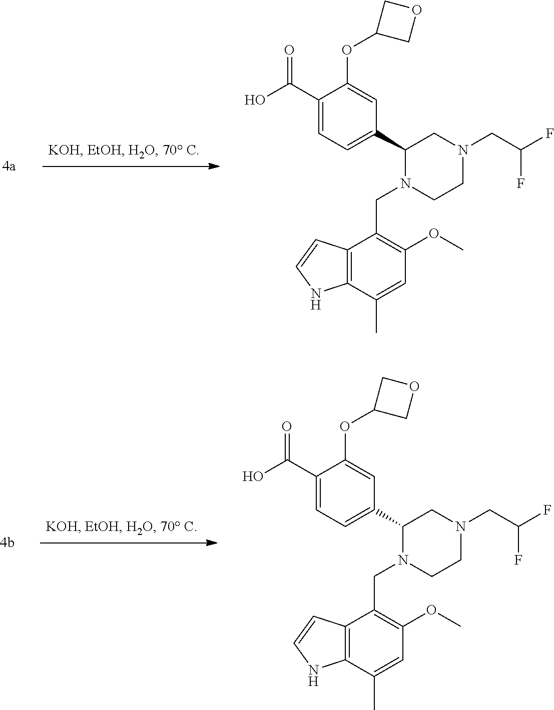

Step 1: tert-Butyl 4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate. To a stirred mixture of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (200 mg, 0.34 mmol) and 3-(4-methylbenzenesulfonyl) oxetane (200 mg, 0.94 mmol) in DMF (5 mL) were added Cesium carbonate (300 mg, 0.92 mmol). The resulting mixture was stirred for additional overnight at 80° C. The resulting mixture was quenched with water and extracted with ethyl acetate for 3 times. The combined organic layers were washed with brine, dried over anhydrous sodium sulfate. After filtration, the filtrate was concentrated under reduced pressure. The residue was purified by silica gel column chromatography, eluted with ethyl acetate/petroleum ether (1/8) to afford the title compound (110 mg, 50.1% yield) as a yellow solid. LCMS (ESI): [M+H]⁺=630.3.

Step 2: tert-Butyl(S)-4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate & tert-Butyl (R)-4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate. tert-Butyl 4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate (120 mg) was applied for further separation by Chiral HPLC.

tert-Butyl(S)-4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate (isomer 1, faster peak) (24 mg, 20.0% yield), LCMS (ESI): [M+H]⁺=630.3. t_{R}=2.89 min (CHIRALPAK IG-3, 4.6*50 mm, 3 μm), Hex (0.1% DEA):Ethanol=80:20, 1.0 mL/min, 254 nm). tert-Butyl (R)-4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate (isomer 2, slower peak) (40 mg, 33.3% yield), LCMS (ESI): [M+H]⁺=630.3. t_{R}=3.58 min (CHIRALPAK IG-3, 4.6*50 mm, 3 μm), Hex (0.1% DEA):Ethanol=80:20, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-(oxetan-3-yloxy)benzoic acid & (R)-4-(4-(2,2-difluoroethyl)-1-((5-methoxy-7-methyl-1H-indol-4-yl)methyl)piperazin-2-yl)-2-(oxetan-3-yloxy)benzoic acid. To a stirred mixture of tert-Butyl(S)-4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate (24 mg, 0.03 mmol), Potassium hydroxide (12 mg, 0.21 mmol) in ethanol (3 mL) and water (0.5 mL) was stirred for 2 h at 70° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. to afford the title compound (10.2 mg, 65.9% yield) as a white solid. LCMS (ESI): [M+H]⁺=516.2, ¹H NMR (300 MHz, Methanol-d₄) δ 7.70 (d, J=7.8 Hz, 1H), 7.30 (d, J=3.1 Hz, 1H), 7.23 (d, J=7.8 Hz, 1H), 6.88-6.81 (m, 1H), 6.75 (s, 1H), 6.34 (d, J=3.1 Hz, 1H), 5.97 (tt, J=55.8, 4.1 Hz, 1H), 5.34 (h, J=5.3 Hz, 1H), 5.10-4.98 (m, 2H), 4.86-4.74 (m, 2H), 4.20 (d, J=11.8 Hz, 2H), 3.93 (d, J=12.6 Hz, 1H), 3.80 (s, 3H), 3.26 (d, J=12.8 Hz, 1H), 3.19-2.95 (m, 3H), 2.93-2.60 (m, 4H), 2.52-2.45 (m, 3H).

To a stirred mixture of tert-Butyl(S)-4-((4-(2,2-difluoroethyl)-2-(4-(methoxycarbonyl)-3-(oxetan-3-yloxy)phenyl)piperazin-1-yl)methyl)-5-methoxy-7-methyl-1H-indole-1-carboxylate (40 mg, 0.05 mmol), Potassium hydroxide (40 mg, 0.63 mmol) in ethanol (3 mL) and water (0.5 mL) was stirred for 2 h at 70° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. to afford the title compound (13.4 mg, 51.9% yield) as a white solid. LCMS (ESI): [M+H]⁺=516.2, ¹H NMR (300 MHz, Methanol-d₄) δ 7.70 (d, J=7.8 Hz, 1H), 7.30 (d, J=3.1 Hz, 1H), 7.23 (d, J=7.8 Hz, 1H), 6.88-6.81 (m, 1H), 6.75 (s, 1H), 6.34 (d, J=3.1 Hz, 1H), 5.97 (tt, J=55.8, 4.1 Hz, 1H), 5.34 (h, J=5.3 Hz, 1H), 5.10-4.98 (m, 2H), 4.86-4.74 (m, 2H), 4.20 (d, J=11.8 Hz, 2H), 3.93 (d, J=12.6 Hz, 1H), 3.80 (s, 3H), 3.26 (d, J=12.8 Hz, 1H), 3.19-2.95 (m, 3H), 2.93-2.60 (m, 4H), 2.52-2.45 (m, 3H).

### Example 11. (S)-8-(4-(2,2-Difluoroethyl)-1-((5-methoxy-7-methyl-1H-indol-4-yl)methyl)piperazin-2-yl)-2,3-dihydrobenzo[b][1,4]dioxine-5-carboxylic acid & (R)-8-(4-(2,2-Difluoroethyl)-1-((5-methoxy-7-methyl-1H-indol-4-yl)methyl)piperazin-2-yl)-2,3-dihydrobenzo[b][1,4]dioxine-5-carboxylic acid

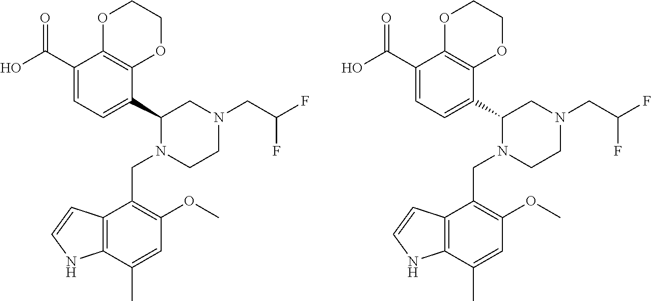 [SMILES: COc1cc(C)c2[nH]ccc2c1CN1CCN(CC(F)F)C[C@@H]1c1ccc(C(=O)O)c2c1OCCO2 ; COc1cc(C)c2[nH]ccc2c1CN1CCN(CC(F)F)C[C@H]1c1ccc(C(=O)O)c2c1OCCO2]

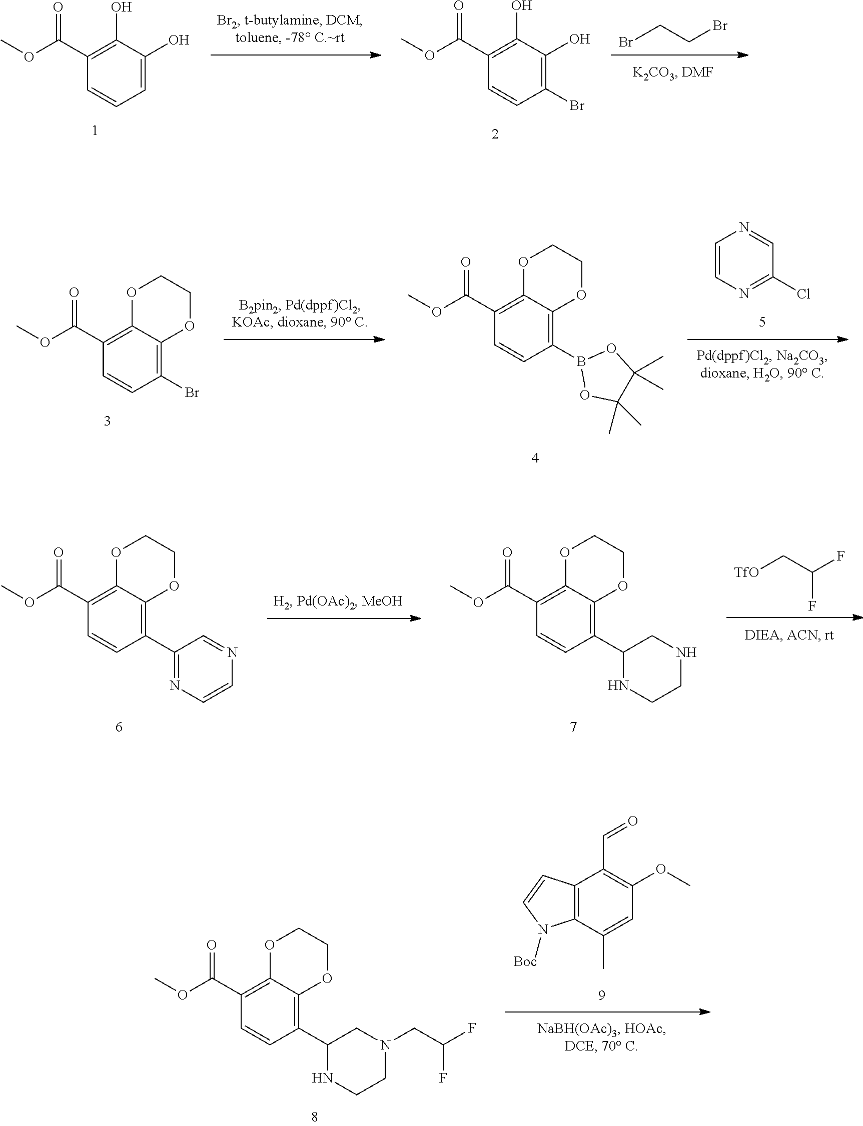

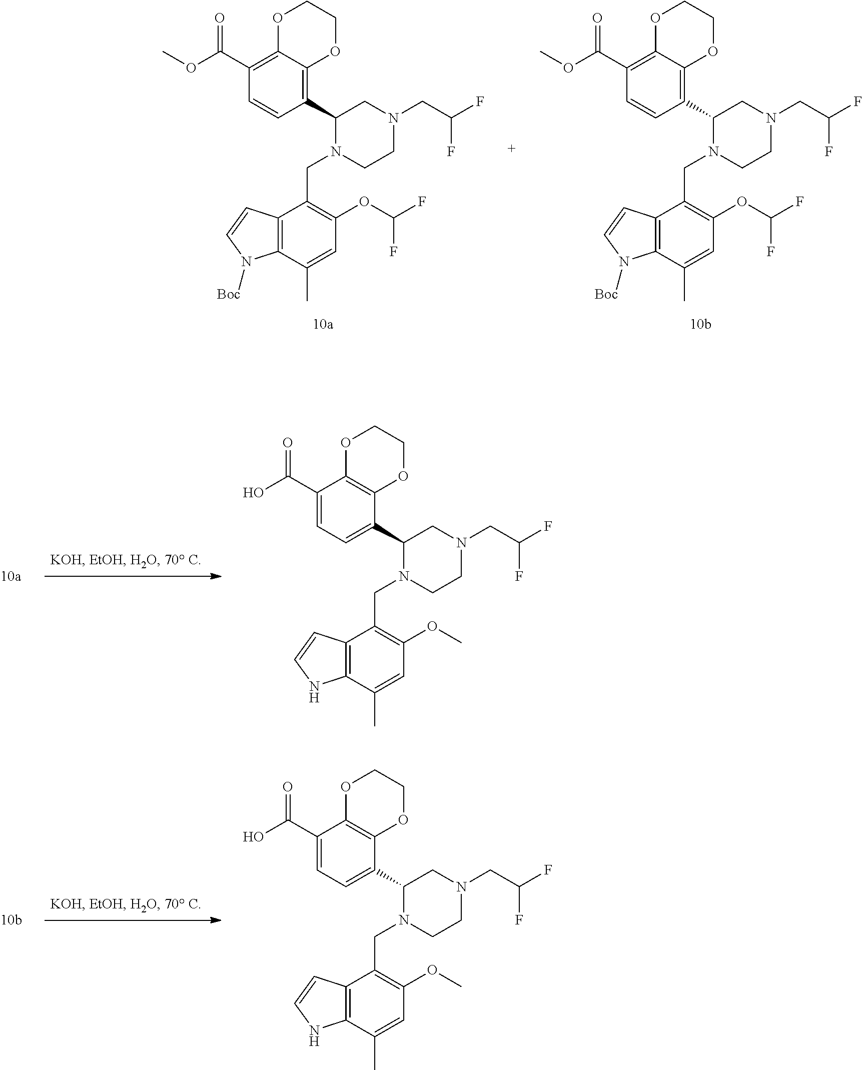

Step 1: Methyl 4-bromo-2,3-dihydroxybenzoate. To a solution of tert-butylamine (12.3 mL, 119 mmol) in toluene (80 mL) and DCM (20 mL) were added bromine (11.40 g, 71.4 mmol) in DCM (20 mL), then the mixture was cooled down to −78° C. and a solution of methyl 2,3-dihydroxy benzoate (10 g, 59.5 mmol) in DCM (20 mL) were added at −78° C. dropwise over 30 min. Then the mixture was warm to room temperature slowly and stirred for 2 h. The residue was washed with 1 M HCl solution. The resulting mixture was extracted with ethyl acetate and washed with brine, dried over anhydrous sodium sulfate. The resulting mixture was concentrated under vacuum. The residue was purified by silica gel column chromatography, eluted with ethyl acetate/petroleum ether=1/9 afford the title compound (4 g, 19.1% yield) as a yellow oil. LCMS (ESI): [M+H]⁺=247.1.

Step 2: Methyl 8-bromo-2,3-dihydrobenzo[b][1,4]dioxine-5-carboxylate. To a solution of methyl 4-bromo-2,3-dihydroxybenzoate (4 g, 11.3 mmol) in DMF (50 mL) was added potassium carbonate (4.7 g, 34.0 mmol) at rt. The mixture was stirred for 30 min. Then 1,2-dibromoethane (2.56 g, 13.6 mmol) was added and the mixture was allowed to warm to 70° C. and stirred for 16 h. The reaction mixture was quenched by water and extracted with ethyl acetate. The resulting mixture was concentrated under vacuum. The residue was purified by reverse phase to afford the title compound (1.7 g, 49.4% yield) as a brown solid. LCMS (ESI): [M+H]⁺=273.2.

Step 3: Methyl 8-(4,4,5,5-tetramethyl-1,3,2-dioxaborolan-2-yl)-2,3-dihydro benzo[b][1,4]dioxine-5-carboxylate. Under nitrogen, a mixture of methyl 8-bromo-2,3-dihydrobenzo[b][1,4]dioxine-5-carboxylate (1.7 g, 6.22 mmol), bis(pinacolato)diboron (2.37 g, 9.33 mmol), potassium acetate (1.8 g, 18.7 mmol) and [1,1′-Bis(diphenylphosphino)ferrocene]dichloropalladium(II) (455 mg, 0.62 mmol) in dioxane (20 mL) were stirred for 2 h at 90° C. The reaction was quenched with water and extracted with ethyl acetate. The combined organic layers were dried over anhydrous sodium sulfate and concentrated under reduced pressure. The residue was purified by silica gel column chromatography, eluted with ethyl acetate/petroleum ether (15%) to afford the title compound (1.5 g, 75.3% yield) as a yellow solid. LCMS (ESI): [M+H]⁺=321.1.

Step 4: Methyl 8-(pyrazin-2-yl)-2,3-dihydrobenzo[b][1,4]dioxine-5-carboxylate. Under nitrogen, a solution of methyl 8-(4,4,5,5-tetramethyl-1,3,2-dioxaborolan-2-yl)-2,3-dihyd robenzo[b][1,4]dioxine-5-carboxylate (1.5 g, 4.7 mmol), 2-chloropyrazine (644 mg, 5.6 mmol), [1,1′-Bis(diphenylphosphino)ferrocene]dichloropalladium(II) (343 mg, 0.469 mmol) and sodium carbonate (1.49 g, 14.0 mmol) in dioxane (20 mL) and water (4 mL) were stirred for 2 h at 90° C. The solvent was removed under vacuum. The residue was purified by silica gel column chromatography, eluted with ethyl acetate/petroleum ether (40%) to afford the title compound (750 mg, 58.8% yield) as an orange solid. LCMS (ESI): [M+H]⁺=273.08.

Step 5: Methyl 8-(piperazin-2-yl)-2,3-dihydrobenzo[b][1,4]dioxine-5-carboxylate. Under hydrogen, a solution of methyl 8-(pyrazin-2-yl)-2,3-dihydrobenzo[b][1,4]dioxine-5-carboxylate (500 mg, 1.8 mmol), Pd/C (250 mg) in methanol (10 mL) was stirred for 2 h at rt. The solids were filtered out. The filtrate was concentrated under reduced pressure to afford the title compound (900 mg crude) as a yellow oil. LCMS (ESI): [M+H]⁺=279.0.

Step 6: Methyl 8-(4-(2,2-difluoroethyl)piperazin-2-yl)-2,3-dihydrobenzo[b][1,4]dioxine-5-carboxylate. To a solution of methyl 8-(piperazin-2-yl)-2,3-dihydrobenzo[b][1,4]dioxine-5-carboxylate (900 mg crude, 1.8 mmol) and 2,2-difluoroethyltrifluoromethanesulfonate (692 mg, 3.2 mmol) in acetonitrile (10 mL) was added N,N-diisopropylethylamine (2.0 g, 16.2 mmol) at 0° C. The mixture was stirred for 1 h at rt. The reaction was quenched with water and extracted with ethyl acetate. The combined organic layers were washed with brine, dried over anhydrous sodium sulfate, and concentrated under reduced pressure. The residue was purified by silica gel column chromatography, eluted with ethyl acetate/petroleum ether (30%) to afford the title compound (1.2 g) as crude and as yellow solids. LCMS (ESI): [M+H]⁺=343.1.

Step 7: tert-butyl(S)-4-((4-(2,2-difluoroethyl)-2-(8-(methoxycarbonyl)-2,3-dihydrobenzo[b][1,4]dioxin-5-yl)piperazin-1-yl)methyl)-5-methoxy-7-methyl-1H-indole-1-carboxylate & tert-butyl (R)-4-((4-(2,2-difluoroethyl)-2-(8-(methoxycarbonyl)-2,3-dihydrobenzo[b][1,4]dioxin-5-yl)piperazin-1-yl)methyl)-5-methoxy-7-methyl-1H-indole-1-carboxylate. To a solution of methyl 8-(4-(2,2-difluoroethyl)piperazin-2-yl)-2,3-dihydrobenzo[b][1,4]dioxine-5-carboxylate (200 mg, 0.58 mmol), tert-butyl 4-formyl-5-methoxy-7-methylindole-1-carboxylate (169 mg, 0.58 mmol) in 1,2-dichloroethane (5 mL) was added acetic acid (42 mg, 0.70 mmol) at room temperature. The mixture was stirred for 1 h at room temperature. Then sodium triacetoxyborohydride (496 mg, 2.34 mmol) was added and the mixture was allowed to warm to 70° C. and stirred for 12 h. The solvent was removed under vacuum. The residue was purified by silica gel column chromatography, eluted with ethyl acetate/petroleum ether (40%) to afford the title compound (200 mg, 55.6% yield) as a yellow oil and as racemate. LCMS (ESI): [M+H]⁺=616.2. The product was applied for further purification by Chiral-Prep-HPLC with the following conditions: Column: CHIRALPAK IG, 2*25 cm, 5 μm; Mobile Phase A: Hex (0.5% 2M NH₃-MeOH)-HPLC, Mobile Phase B: Ethanol-HPLC; Flow rate: 20 mL/min; Gradient: 20% B to 20% B in 22 min; Wave Length: 220/254 nm; RT1(min): 10.423; RT2(min): 15.59; Sample Solvent: Ethanol-HPLC; Injection Volume: 1 mL; Number Of Runs: 3.

tert-butyl(S)-4-((4-(2,2-difluoroethyl)-2-(8-(methoxycarbonyl)-2,3-dihydrobenzo[b][1,4]dioxin-5-yl)piperazin-1-yl)methyl)-5-methoxy-7-methyl-1H-indole-1-carboxylate (80 mg, 40% yield, faster peak, isomer 1), LCMS (ESI): [M+H]⁺=616.2, tR=2.05 min (CHIRALPAK IG-3 (0.46*5 cm,3 μm), Hex (0.1% DEA):Ethanol=80:20, 1.0 mL/min, 254 nm). tert-butyl (R)-4-((4-(2,2-difluoroethyl)-2-(8-(methoxycarbonyl)-2,3-dihydrobenzo[b][1,4]dioxin-5-yl)piperazin-1-yl)methyl)-5-methoxy-7-methyl-1H-indole-1-carboxylate (80 mg, 40% yield, slower peak, isomer 2), LCMS (ESI): [M+H]⁺=616.2, tR=3.24 min (CHIRALPAK IG-3 (0.46*5 cm,3 μm), Hex (0.1% DEA):Ethanol=80:20, 1.0 mL/min, 254 nm).

Step 8: (S)-8-(4-(2,2-Difluoroethyl)-1-((5-methoxy-7-methyl-1H-indol-4-yl)methyl)piperazin-2-yl)-2,3-dihydrobenzo[b][1,4]dioxine-5-carboxylic acid & (R)-8-(4-(2,2-Difluoroethyl)-1-((5-methoxy-7-methyl-1H-indol-4-yl)methyl)piperazin-2-yl)-2,3-dihydrobenzo[b][1,4]dioxine-5-carboxylic acid.

To a solution of(S)-4-((4-(2,2-difluoroethyl)-2-(8-(methoxycarbonyl)-2,3-dihydrobenzo[b][1,4]dioxin-5-yl)piperazin-1-yl)methyl)-5-methoxy-7-methyl-1H-indole-1-carboxylate (80 mg, 0.13 mmol, faster peak, isomer 1) in Ethanol (2 mL) and water (1 mL) was added potassium hydroxide (29 mg, 0.52 mmol) at room temperature. The mixture was stirred for 1 h at 90° C. The pH was adjusted to 6 with sat. citric acid. The resulting mixture was concentrated under vacuum. The residue was purified by reversed-phase flash chromatography (ACN/10 mmol NH₄HCO₃) to afford the title compound (35.8 mg, 54.8% yield) as a white solid. LCMS (ESI): [M+H]⁺=502.2, ¹H NMR (300 MHz, DMSO-d₆) δ 10.84 (s, 1H), 7.31-7.15 (m, 2H), 7.07 (d, J=8.0 Hz, 1H), 6.66 (s, 1H), 6.52-6.42 (m, 1H), 6.34-5.87 (m, 1H), 4.41-4.12 (m, 4H), 3.72 (s, 6H), 3.20 (d, J=11.8 Hz, 1H), 2.86-2.70 (m, 2H), 2.7-2.6 (m, 2H), 2.59 (s, 1H), 2.43 (s, 3H), 2.16 (t, J=10.7 Hz, 3H).

To a solution of (R)-4-((4-(2,2-difluoroethyl)-2-(8-(methoxycarbonyl)-2,3-dihydrobenzo[b][1,4]dioxin-5-yl)piperazin-1-yl)methyl)-5-methoxy-7-methyl-1H-indole-1-carboxylate (80 mg, 0.13 mmol, slower peak, isomer 2) in Ethanol (2 mL) and water (1 mL) was added potassium hydroxide (29 mg, 0.52 mmol) at room temperature. The mixture was stirred for 1 h at 90° C. The pH was adjusted to 6 with sat. citric acid. The resulting mixture was concentrated under vacuum. The residue was purified by reversed-phase flash chromatography (ACN/10 mmol NH₄HCO₃) to afford the title compound (35.7 mg, 54.7% yield) as a white solid. LCMS (ESI): [M+H]⁺=502.2, ¹H NMR (300 MHz, DMSO-d₆) δ 10.84 (s, 1H), 7.31-7.15 (m, 2H), 7.07 (d, J=8.0 Hz, 1H), 6.66 (s, 1H), 6.52-6.42 (m, 1H), 6.34-5.87 (m, 1H), 4.41-4.12 (m, 4H), 3.72 (s, 6H), 3.20 (d, J=11.8 Hz, 1H), 2.86-2.70 (m, 2H), 2.7-2.6 (m, 2H), 2.59 (s, 1H), 2.43 (s, 3H), 2.16 (t, J=10.7 Hz, 3H).

### Example 12. (S)-2-(Dimethylamino)-4-(1-((5-methoxy-7-methyl-1H-indol-4-yl)methyl)-4-(3,3,3-trifluoropropyl)piperazin-2-yl)benzoic acid & (R)-2-(Dimethylamino)-4-(1-((5-methoxy-7-methyl-1H-indol-4-yl)methyl)-4-(3,3,3-trifluoropropyl)piperazin-2-yl)benzoic acid

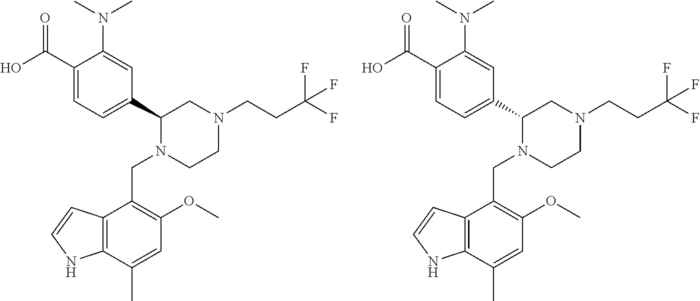 [SMILES: COc1cc(C)c2[nH]ccc2c1CN1CCN(CCC(F)(F)F)C[C@@H]1c1ccc(C(=O)O)c(N(C)C)c1 ; COc1cc(C)c2[nH]ccc2c1CN1CCN(CCC(F)(F)F)C[C@H]1c1ccc(C(=O)O)c(N(C)C)c1]

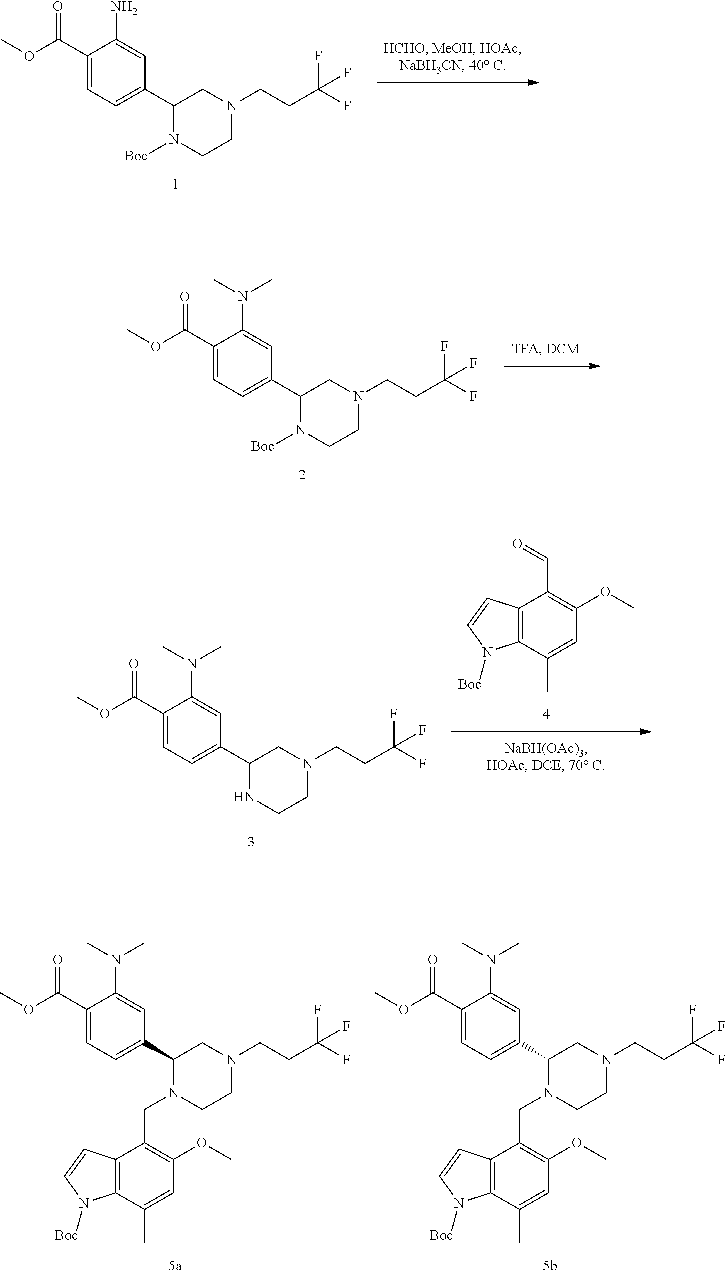

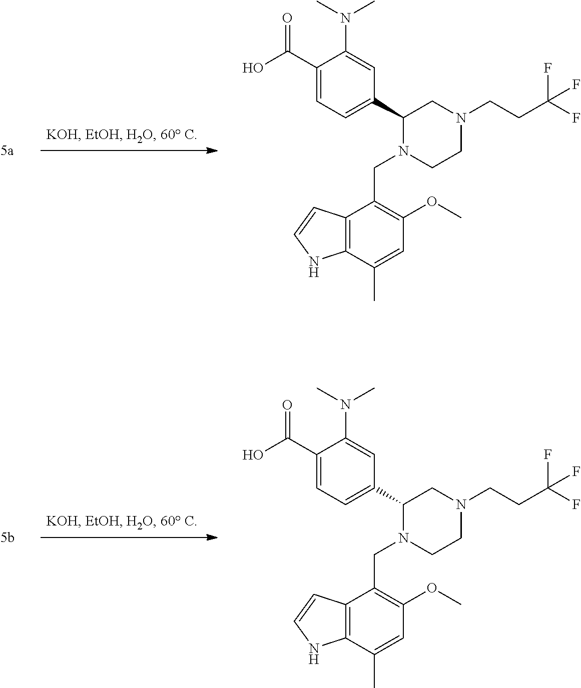 [SMILES: COc1cc(C)c2[nH]ccc2c1CN1CCN(CCC(F)(F)F)C[C@@H]1c1ccc(C(=O)O)c(N(C)C)c1 ; COc1cc(C)c2[nH]ccc2c1CN1CCN(CCC(F)(F)F)C[C@H]1c1ccc(C(=O)O)c(N(C)C)c1]

Step 1: tert-Butyl 2-(3-(dimethylamino)-4-(methoxycarbonyl)phenyl)-4-(3,3,3-trifluoropropyl)piperazine-1-carboxylate. A mixture of tert-butyl 2-(3-amino-4-(methoxycarbonyl)phenyl)-4-(3,3,3-trifluoropropyl)piperazine-1-carboxylate (0.5 g, 1.16 mmol), HOAc (0.3 g, 5.00 mmol), NaBH₃CN (0.6 g, 9.55 mmol) and 37 wt. % HCHO in water (3 mL) in MeOH (10 mL) was stirred for overnight at 40° C. The solvent was removed under vacuum. The resulting mixture was dissolved in ethyl acetate and washed with brine. The organic layer was dried over anhydrous sodium sulfate. After filtration, the filtrate was concentrated under reduced pressure to afford the title compound (629 mg, 94.0% yield) as a yellow solid. LCMS (ESI): [M+H]⁺=460.2.

Step 2: Methyl 2-(dimethylamino)-4-(4-(3,3,3-trifluoropropyl)piperazin-2-yl)benzoate. A mixture of tert-Butyl 2-(3-(dimethylamino)-4-(methoxycarbonyl)phenyl)-4-(3,3,3-trifluoropropyl)piperazine-1-carboxylate (609 mg, 1.32 mmol) in dichloromethane (10 mL) was added trifluoroacetic acid (5 mL) and stirred for 2 h at room temperature. The resulting mixture was concentrated under reduced pressure. The residue was purified by reverse phase flash with ACN/10 mM NH₄HCO₃ in water to afford the title compound (430 mg, 77.6% yield) as a yellow solid. LCMS (ESI): [M+H]⁺=360.1.

Step 3: tert-Butyl(S)-4-((2-(3-(dimethylamino)-4-(methoxycarbonyl)phenyl)-4-(3,3,3-trifluoropropyl)piperazin-1-yl)methyl)-5-methoxy-7-methyl-1H-indole-1-carboxylate & tert-butyl (R)-4-((2-(3-(dimethylamino)-4-(methoxycarbonyl)phenyl)-4-(3,3,3-trifluoropropyl)piperazin-1-yl)methyl)-5-methoxy-7-methyl-1H-indole-1-carboxylate. A solution of methyl 2-(dimethylamino)-4-(4-(3,3,3-trifluoropropyl)piperazin-2-yl)benzoate (300 mg, 0.84 mmol), tert-butyl 4-formyl-5-methoxy-7-methylindole-1-carboxylate (361 mg, 1.25 mmol), HOAc (150 mg, 2.50 mmol) and sodium triacetoxyborohydride (354 mg, 1.67 mmol) in dichloroethane (3 mL) was stirred for overnight at 70° C. The solvent was removed under reduced pressure. The crude product was purified by reverse phase and Chiral HPLC to afford the title compounds.

tert-Butyl(S)-4-((2-(3-(dimethylamino)-4-(methoxycarbonyl)phenyl)-4-(3,3,3-trifluoropropyl)piperazin-1-yl)methyl)-5-methoxy-7-methyl-1H-indole-1-carboxylate (105 mg, 19.9% yield, faster peak, isomer 1), LCMS (ESI): [M+H]⁺=633.2, tR=3.122 min (CHIRALPAK IF-3, 0.46*5 cm,3 um, Hex (0.1% DEA):Ethanol=98:2, 1.0 mL/min, 254 nm).

tert-butyl (R)-4-((2-(3-(dimethylamino)-4-(methoxycarbonyl)phenyl)-4-(3,3,3-trifluoropropyl)piperazin-1-yl)methyl)-5-methoxy-7-methyl-1H-indole-1-carboxylate (106 mg, 20.0% yield, slower peak, isomer 2), LCMS (ESI): [M+H]⁺=633.2, tR=3.72 min (CHIRALPAK IF-3, 0.46*5 cm,3 um, Hex (0.1% DEA):Ethanol=98:2, 1.0 mL/min, 254 nm).

Step 4: (S)-2-(Dimethylamino)-4-(1-((5-methoxy-7-methyl-1H-indol-4-yl)methyl)-4-(3,3,3-trifluoropropyl)piperazin-2-yl)benzoic acid & (R)-2-(Dimethylamino)-4-(1-((5-methoxy-7-methyl-1H-indol-4-yl)methyl)-4-(3,3,3-trifluoropropyl)piperazin-2-yl)benzoic acid.

A solution of tert-butyl (S)-4-((2-(3-(dimethylamino)-4-(methoxycarbonyl)phenyl)-4-(3,3,3-trifluoropropyl)piperazin-1-yl)methyl)-5-methoxy-7-methyl-1H-indole-1-carboxylate (95 mg, 0.15 mmol, faster peak, isomer 1) and Potassium hydroxide (84 mg, 1.50 mmol) in water (1.5 mL) and Ethanol (3 mL) was stirred for overnight at 60° C. The crude product was purified by reverse phase flash with ACN/10 mM NH₄HCO₃ to afford the title compound (42 mg, 53.8% yield) as a white solid. LCMS (ESI): [M+H]⁺=519.2. ¹H NMR (300 MHz, DMSO-d₆) δ 10.86 (s, 1H), 7.99 (d, J=8.0 Hz, 1H), 7.79 (s, 1H), 7.58 (d, J=8.0 Hz, 1H), 7.26 (t, J=2.8 Hz, 1H), 6.67 (s, 1H), 6.45 (m, 1H), 3.72 (s, 3H), 3.58 (d, J=11.9 Hz, 1H), 3.45-3.38 (m, 1H), 3.32-3.25 (d, J=11.9 Hz, 2H), 2.85 (s, 6H), 2.81-2.71 (m, 2H), 2.69-2.60 (d, J=11.4 Hz, 1H), 2.49-2.45 (m, 2H), 2.42 (s, 3H), 2.41-2.34 (m, 1H), 2.28-2.15 (m, 1H), 2.15-2.03 (m, 1H), 2.03-1.94 (m, 1H).

A solution of tert-butyl (R)-4-((2-(3-(dimethylamino)-4-(methoxycarbonyl)phenyl)-4-(3,3,3-trifluoropropyl)piperazin-1-yl)methyl)-5-methoxy-7-methyl-1H-indole-1-carboxylate (96 mg, 0.15 mmol, slower peak, isomer 2) and Potassium hydroxide (84 mg, 1.50 mmol) in water (1.5 mL) and Ethanol (3 mL) was stirred for overnight at 60° C. The crude product was purified by reverse phase flash with ACN/10 mM NH₄HCO₃ to afford the title compound (41 mg, 52.8% yield) as a white solid. LCMS (ESI): [M+H]⁺=519.2. ¹H NMR (300 MHz, DMSO-d₆) δ 10.86 (s, 1H), 7.99 (d, J=8.0 Hz, 1H), 7.79 (s, 1H), 7.58 (d, J=8.0 Hz, 1H), 7.26 (t, J=2.8 Hz, 1H), 6.67 (s, 1H), 6.45 (m, 1H), 3.72 (s, 3H), 3.58 (d, J=11.9 Hz, 1H), 3.45-3.38 (m, 1H), 3.32-3.25 (d, J=11.9 Hz, 2H), 2.85 (s, 6H), 2.81-2.71 (m, 2H), 2.69-2.60 (d, J=11.4 Hz, 1H), 2.49-2.45 (m, 2H), 2.42 (s, 3H), 2.41-2.34 (m, 1H), 2.28-2.15 (m, 1H), 2.15-2.03 (m, 1H), 2.03-1.94 (m, 1H).

### Example 13. (S)-4-(4-(2,2-Difluoroethyl)-1-((5-methoxy-7-methyl-1H-indol-4-yl)methyl)piperazin-2-yl)-2-(oxetan-3-ylamino)benzoic acid & (R)-4-(4-(2,2-Difluoroethyl)-1-((5-methoxy-7-methyl-1H-indol-4-yl)methyl)piperazin-2-yl)-2-(oxetan-3-ylamino)benzoic acid

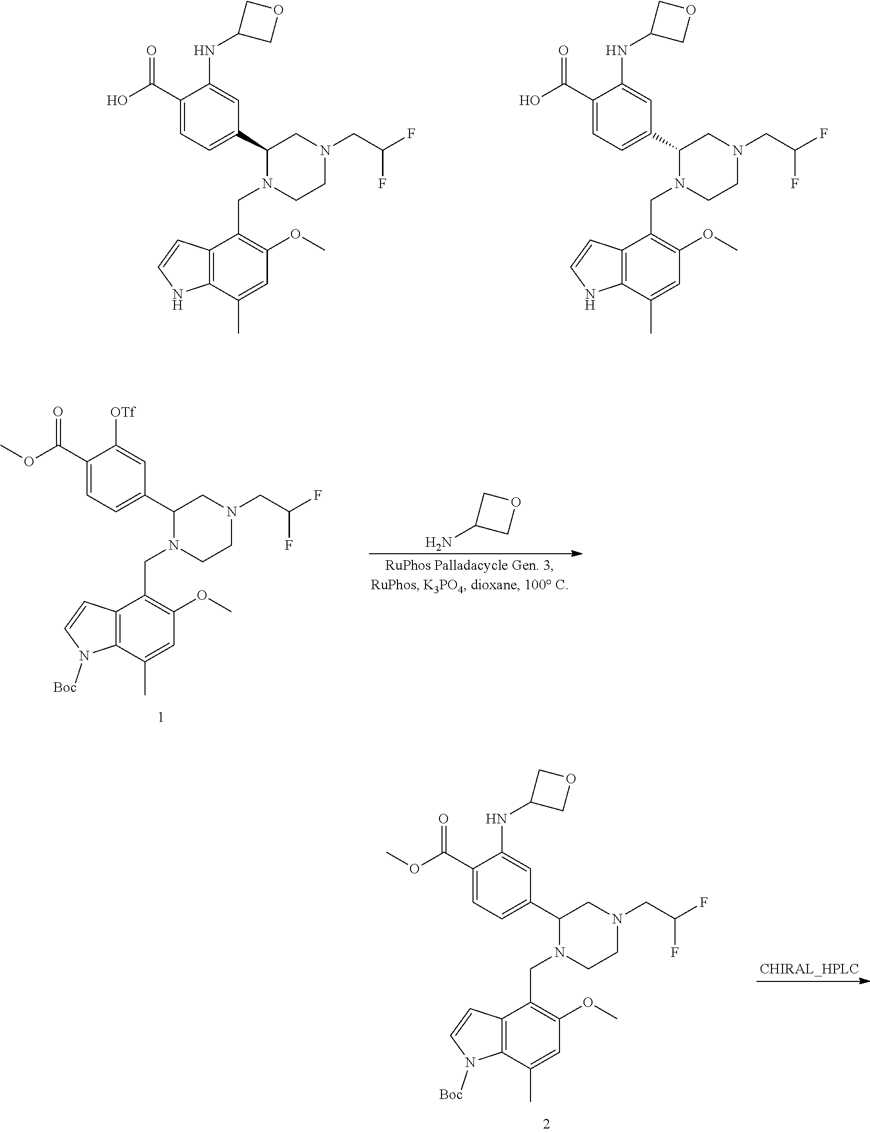 [SMILES: COc1cc(C)c2[nH]ccc2c1CN1CCN(CC(F)F)C[C@@H]1c1ccc(C(=O)O)c(NC2COC2)c1 ; COc1cc(C)c2[nH]ccc2c1CN1CCN(CC(F)F)C[C@H]1c1ccc(C(=O)O)c(NC2COC2)c1 ; COC(=O)c1ccc(C2CN(CC(F)F)CCN2Cc2c(OC)cc(C)c3c2ccn3C(=O)OC(C)(C)C)cc1OS(=O)(=O)C(F)(F)F ; NC1COC1 ; COC(=O)c1ccc(C2CN(CC(F)F)CCN2Cc2c(OC)cc(C)c3c2ccn3C(=O)OC(C)(C)C)cc1NC1COC1]

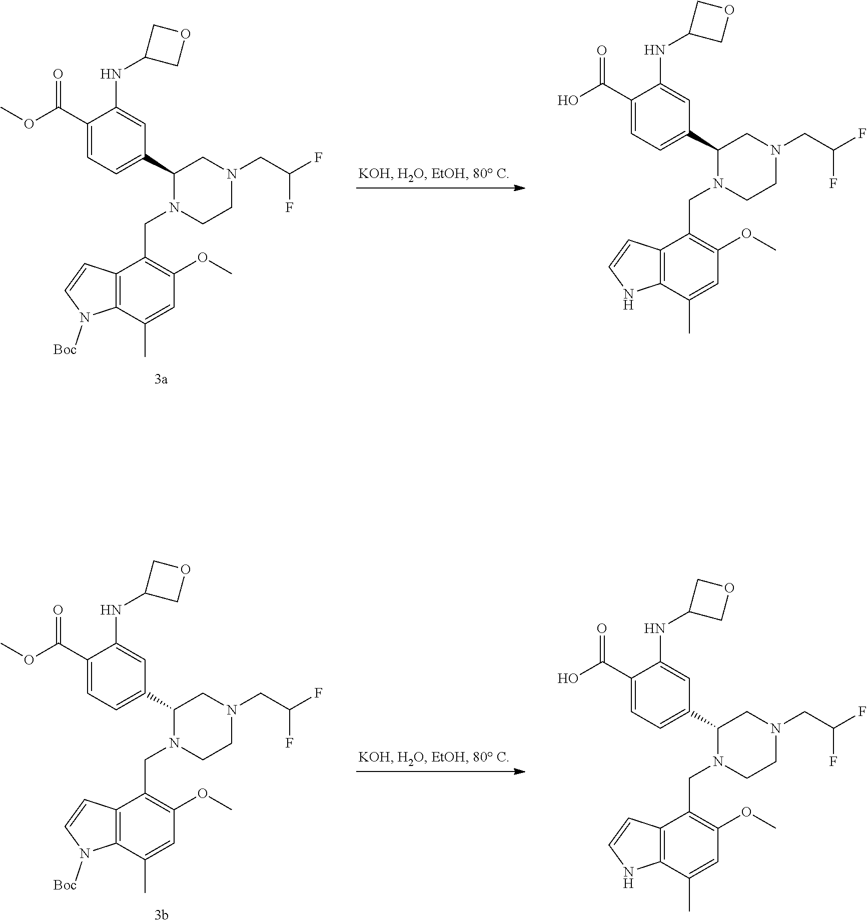 [SMILES: COC(=O)c1ccc([C@H]2CN(CC(F)F)CCN2Cc2c(OC)cc(C)c3c2ccn3C(=O)OC(C)(C)C)cc1NC1COC1 ; COc1cc(C)c2[nH]ccc2c1CN1CCN(CC(F)F)C[C@@H]1c1ccc(C(=O)O)c(NC2COC2)c1 ; COC(=O)c1ccc([C@@H]2CN(CC(F)F)CCN2Cc2c(OC)cc(C)c3c2ccn3C(=O)OC(C)(C)C)cc1NC1COC1 ; COc1cc(C)c2[nH]ccc2c1CN1CCN(CC(F)F)C[C@H]1c1ccc(C(=O)O)c(NC2COC2)c1]

Step 1: tert-Butyl 4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate. A solution of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (500 mg, 0.71 mmol) and oxetan-3-amine (150 mg, 2.06 mmol) in 1,4-dioxane (1 mL) was added RuPhos Pd G₃ (148 mg, 0.18 mmol), RuPhos (99 mg, 0.21 mmol) and potassium phosphate tribasic (299 mg, 1.41 mmol) and stirred for 2 h at 100° C. under a nitrogen atmosphere. The resulting mixture was concentrated under reduced pressure. The residue was purified by silica gel column chromatography, eluted with ethyl acetate/petroleum ether (1/5) to afford the title compound (300 mg, 0.48 mmol) as a yellow solid. LCMS (ESI): [M+H]⁺=629.3.

Step 2: tert-Butyl(S)-4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (isomer 1, faster peak) & tert-Butyl (R)-4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (isomer 2, slower peak). tert-Butyl 4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (300 mg, 0.48 mmol) was applied for separation by Chiral HPLC with the following condition: Column: CHIRALPAK IG, 2*25 cm, 5 μm; Mobile Phase A: Hex (0.5% 2M NH₃-MeOH)-HPLC, Mobile Phase B: Ethanol-HPLC; Flow rate: 20 mL/min; Gradient: 20% B to 20% B in 15 min; Wave Length: 220/254 nm; RT1(min): 10.888; RT2(min): 13.264; Sample Solvent: Ethanol-HPLC; Injection Volume: 0.4 mL; Number of Runs: 22.

tert-Butyl(S)-4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (isomer 1, faster peak) (147 mg, 36.7% yield). LCMS (ESI): [M+H]⁺=629.3. tR=2.04 min (CHIRALPAK IG-3 (: 4.6*50 mm, 3 μm),: Hex (0.1% DEA):Ethanol=80:20, 1.0 mL/min, 254 nm).

tert-Butyl (R)-4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (isomer 2, slower peak) (135 mg, 33.7% yield). LCMS (ESI): [M+H]⁺=629.3. tR=2.68 min (CHIRALPAK IG-3 (4.6*50 mm, 3 μm), Hex (0.1% DEA):Ethanol=80:20, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-(oxetan-3-ylamino)benzoic acid & (R)-4-(4-(2,2-Difluoroethyl)-1-((5-methoxy-7-methyl-1H-indol-4-yl)methyl)piperazin-2-yl)-2-(oxetan-3-ylamino)benzoic acid. To a stirred solution of tert-butyl (S)-4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (130 mg, 0.21 mmol, faster peak) and Potassium hydroxide (130 mg, 2.31 mmol) in water (1 mL, 55.5 mmol,) and Ethanol (5 mL, 86.07 mmol). The resulting mixture was stirred for additional 2 h at 80° C. The reaction mixture was acidified to pH=6 with sat. citric acid solution. The solvent was removed under vacuum. The residue was purified by reversed-phase flash chromatography with MeCN/10 mmol/L NH₄HCO₃ afford (S)-4-(4-(2,2-Difluoroethyl)-1-((5-methoxy-7-methyl-1H-indol-4-yl)methyl)piperazin-2-yl)-2-(oxetan-3-ylamino)benzoic acid (53.5 mg, 50.1% yield) as a white solid. LCMS (ESI) [M+H]⁺=515.2, ¹H NMR (300 MHz, Methanol-d₄) δ 8.03 (d, J=8.0 Hz, 1H), 7.27 (d, J=3.1 Hz, 1H), 6.98-6.50 (m, 3H), 6.32 (d, J=3.1 Hz, 1H), 5.97 (t, J=55.9 Hz, 1H), 5.03 (s, 2H), 4.80 (q, J=7.0, 6.4 Hz, 1H), 4.60 (d, J=6.3 Hz, 2H), 4.18 (d, J=12.4 Hz, 1H), 4.03 (d, J=10.8 Hz, 1H), 3.86 (d, J=12.5 Hz, 1H), 3.77 (s, 3H), 3.22 (d, J=12.4 Hz, 1H), 3.02 (t, J=13.1 Hz, 3H), 2.90-2.55 (m, 4H), 2.49 (s, 3H).

To a stirred solution of tert-butyl (R)-4-((4-(2,2-difluoroethyl)-2-(4-(methoxycarbonyl)-3-(oxetan-3-ylamino)phenyl)piperazin-1-yl)methyl)-5-methoxy-7-methyl-1H-indole-1-carboxylate (130 mg, 0.21 mmol, slower peak) and Potassium hydroxide (130 mg, 2.31 mmol) in water (1 mL, 55.5 mmol,) and Ethanol (5 mL, 86.07 mmol). The resulting mixture was stirred for additional 2 h at 80° C. The reaction mixture was acidified to pH=6 with sat. citric acid solution. The solvent was removed under vacuum. The residue was purified by reversed-phase flash chromatography with MeCN/10 mmol/L NH₄HCO₃ afford(S)-4-(4-(2,2-Difluoroethyl)-1-((5-methoxy-7-methyl-1H-indol-4-yl)methyl)piperazin-2-yl)-2-(oxetan-3-ylamino)benzoic acid (68.6 mg, 64.1% yield) as a white solid. LCMS (ESI) [M+H]⁺=515.2, ¹H NMR (300 MHz, Methanol-d₄) δ 8.03 (d, J=8.0 Hz, 1H), 7.27 (d, J=3.1 Hz, 1H), 6.98-6.50 (m, 3H), 6.32 (d, J=3.1 Hz, 1H), 5.97 (t, J=55.9 Hz, 1H), 5.03 (s, 2H), 4.80 (q, J=7.0, 6.4 Hz, 1H), 4.60 (d, J=6.3 Hz, 2H), 4.18 (d, J=12.4 Hz, 1H), 4.03 (d, J=10.8 Hz, 1H), 3.86 (d, J=12.5 Hz, 1H), 3.77 (s, 3H), 3.22 (d, J=12.4 Hz, 1H), 3.02 (t, J=13.1 Hz, 3H), 2.90-2.55 (m, 4H), 2.49 (s, 3H).

### Example 14. (S)-2-Chloro-4-(1-((5-methoxy-7-methyl-1H-indol-4-yl)methyl)-4-(3,3,3-trifluoropropyl)piperazin-2-yl)benzoic acid & (R)-2-Chloro-4-(1-((5-methoxy-7-methyl-1H-indol-4-yl)methyl)-4-(3,3,3-trifluoropropyl)piperazin-2-yl)benzoic acid

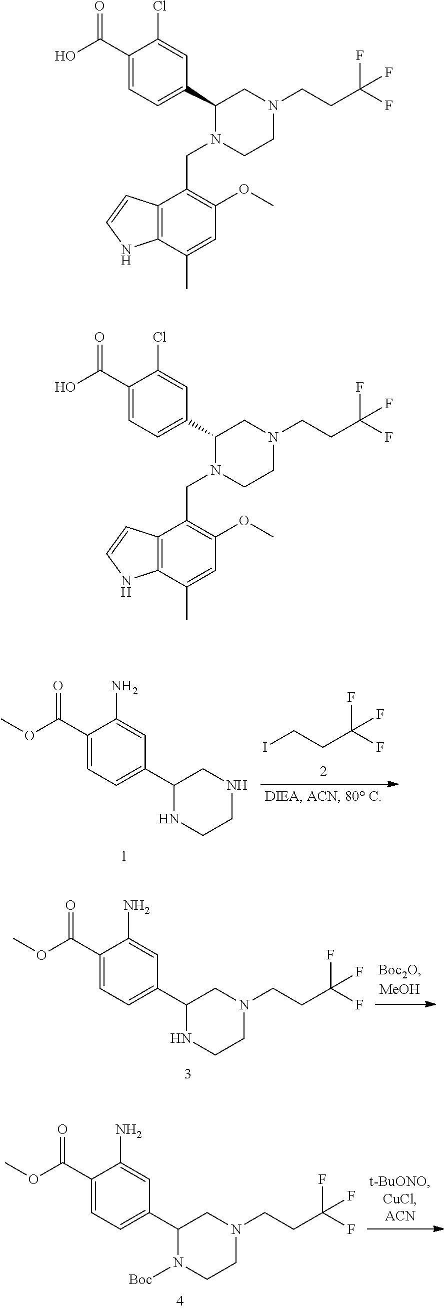 [SMILES: COc1cc(C)c2[nH]ccc2c1CN1CCN(CCC(F)(F)F)C[C@@H]1c1ccc(C(=O)O)c(Cl)c1 ; COc1cc(C)c2[nH]ccc2c1CN1CCN(CCC(F)(F)F)C[C@H]1c1ccc(C(=O)O)c(Cl)c1 ; COC(=O)c1ccc(C2CNCCN2)cc1N ; FC(F)(F)CCI ; COC(=O)c1ccc(C2CN(CCC(F)(F)F)CCN2)cc1N ; COC(=O)c1ccc(C2CN(CCC(F)(F)F)CCN2C(=O)OC(C)(C)C)cc1N]

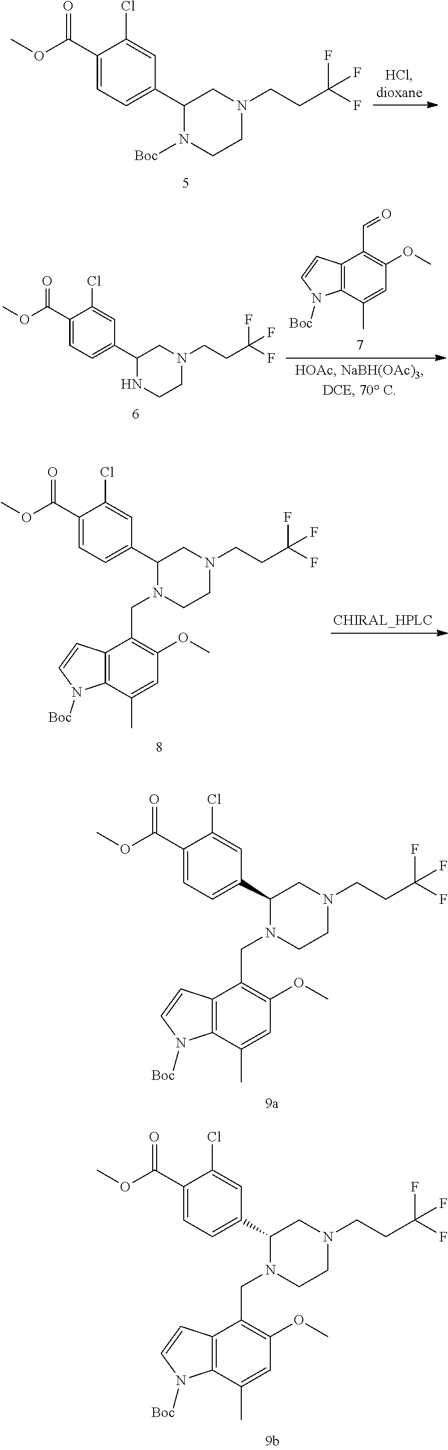

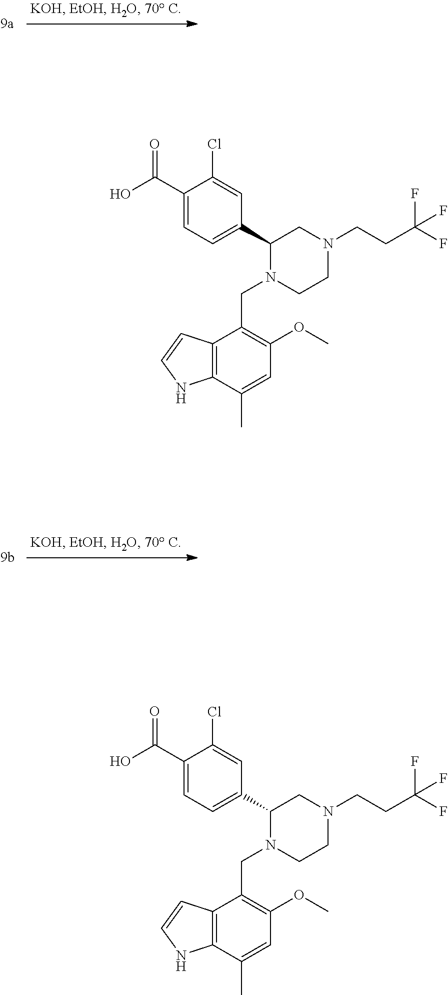 [SMILES: COc1cc(C)c2[nH]ccc2c1CN1CCN(CCC(F)(F)F)C[C@@H]1c1ccc(C(=O)O)c(Cl)c1 ; COc1cc(C)c2[nH]ccc2c1CN1CCN(CCC(F)(F)F)C[C@H]1c1ccc(C(=O)O)c(Cl)c1]

Step 1: Methyl 2-amino-4-(4-(3,3,3-trifluoropropyl)piperazin-2-yl)benzoate. To a stirred mixture of methyl 2-amino-4-(piperazin-2-yl)benzoate (2 g, 8.50 mmol) and 1,1,1-trifluoro-3-iodopropane (2.8 g, 12.5 mmol) in ACN (10 mL) were added N,N-diisopropylethylamine (3.28 g, 25.41 mmol). The resulting mixture was stirred for additional 3 h at 80° C. The resulting mixture was concentrated under reduced pressure. The residue was purified by silica gel column chromatography, eluted with dichloromethane/methanol (10:1) to afford the title compound (1.5 g, 53.3% yield) as a yellow oil. LCMS (ESI): [M+H]⁺=332.2

Step 2: tert-Butyl 2-(3-amino-4-(methoxycarbonyl)phenyl)-4-(3,3,3-trifluoropropyl)piperazine-1-carboxylate. To a stirred mixture of methyl Methyl 2-amino-4-(4-(3,3,3-trifluoropropyl)piperazin-2-yl)benzoate (1.5 g, 4.52 mmol) and Di-tert-butyl dicarbonate (1.20 g, 5.47 mmo) in MeOH (10 mL). The resulting mixture was stirred for additional 1 h at room temperature. The resulting mixture was concentrated under reduced pressure. The residue was purified by silica gel column chromatography, eluted with petroleum ether/ethyl acetate (8:1) to afford the title compound (1.6 g, 81.92%) as a white oil. LCMS (ESI): [M+H]⁺=432.2

Step 3: tert-Butyl 2-(3-chloro-4-(methoxycarbonyl)phenyl)-4-(3,3,3-trifluoropropyl)piperazine-1-carboxylate. To a stirred mixture of tert-Butyl 2-(3-amino-4-(methoxycarbonyl)phenyl)-4-(3,3,3-trifluoropropyl)piperazine-1-carboxylate (500 mg, 1.15 mmol) in ACN (5 mL) was added CuCl (150 mg, 1.51 mmol) and 3-methylbutyl nitrite (200 mg, 1.70 mmol). The resulting mixture was stirred for additional 1 h at room temperature. The resulting mixture was quenched with water and extracted with ethyl acetate for 3 times. The combined organic layers were washed with brine, dried over anhydrous sodium sulfate. After filtration, the filtrate was concentrated under reduced pressure. The residue was purified by silica gel column chromatography, eluted with petroleum ether/ethyl acetate (9:1) to afford the title compound (400 mg, 76.6% yield) as a yellow oil. LCMS (ESI): [M+H]⁺=451.2

Step 4: Methyl 2-chloro-4-(4-(3,3,3-trifluoropropyl)piperazin-2-yl)benzoate. To a stirred mixture of tert-Butyl 2-(3-chloro-4-(methoxycarbonyl)phenyl)-4-(3,3,3-trifluoropropyl)piperazine-1-carboxylate (400 mg, 0.88 mmol) in DCM (2 mL) was added 4 M HCl in 1,4-dioxane (2 mL, 2 mmol). The resulting mixture was stirred for 1 h at room temperature. The resulting mixture was concentrated under reduced pressure. The residue was purified by silica gel column chromatography, eluted with dichloromethane/methanol (9:1) to afford the title compound (260 mg, 83.6% yield) as a yellow oil. LCMS (ESI): [M+H]⁺=351.1.

Step 5: tert-Butyl 4-((2-(3-chloro-4-(methoxycarbonyl)phenyl)-4-(3,3,3-trifluoropropyl)piperazin-1-yl)methyl)-5-methoxy-7-methyl-1H-indole-1-carboxylate. To a stirred mixture of methyl 2-chloro-4-(4-(3,3,3-trifluoropropyl)piperazin-2-yl)benzoate (250 mg, 0.71 mmol) and tert-butyl 4-formyl-5-methoxy-7-methylindole-1-carboxylate (250 mg, 0.86 mmol) in dichloroethane (5 mL) were added Sodium triacetoxyborohydride (300 mg, 1.41 mmol) and HOAc (50 mg, 0.83 mmol). The resulting mixture was stirred for additional 3 h at 45° C. The resulting mixture was concentrated under reduced pressure. The residue was purified by reversed-phase flash chromatography with the following conditions: column, C18 silica gel; mobile phase, MeCN in Water (10 mmol/L NH₄HCO₃), 70% to 85% gradient in 10 min; detector, UV 254 nm. to afford the title compound (230 mg, 51.7% yield) as a yellow solid. LCMS (ESI): [M+H]⁺=624.2.

Step 6: tert-Butyl (S)-4-((2-(3-chloro-4-(methoxycarbonyl)phenyl)-4-(3,3,3-trifluoropropyl)piperazin-1-yl)methyl)-5-methoxy-7-methyl-1H-indole-1-carboxylate (isomer 1, faster peak) & tert-Butyl (R)-4-((2-(3-chloro-4-(methoxycarbonyl)phenyl)-4-(3,3,3-trifluoropropyl)piperazin-1-yl)methyl)-5-methoxy-7-methyl-1H-indole-1-carboxylate (isomer 2, slower peak).

tert-Butyl 4-((2-(3-chloro-4-(methoxycarbonyl)phenyl)-4-(3,3,3-trifluoropropyl)piperazin-1-yl)methyl)-5-methoxy-7-methyl-1H-indole-1-carboxylate (190 mg) was applied for further separation by Chiral HPLC.

tert-Butyl(S)-4-((2-(3-chloro-4-(methoxycarbonyl)phenyl)-4-(3,3,3-trifluoropropyl)piperazin-1-yl)methyl)-5-methoxy-7-methyl-1H-indole-1-carboxylate (isomer 1, faster peak) (68 mg, 31.5% yield). LCMS (ESI): [M+H]⁺=624.2. tR=2.00 min (Lux Cellulose-4 4.6*50 mm, 3 um, Hex (0.1% DEA):IPA=98:2, 1.0 mL/min, 254 nm).

tert-Butyl (R)-4-((2-(3-chloro-4-(methoxycarbonyl)phenyl)-4-(3,3,3-trifluoropropyl)piperazin-1-yl)methyl)-5-methoxy-7-methyl-1H-indole-1-carboxylate (isomer 1, slower peak) (63 mg, 33.2% yield). LCMS (ESI): [M+H]⁺=624.2, tR=2.71 min (Lux Cellulose-4 4.6*50 mm, 3 um), Hex (0.1% DEA):IPA=98:2, 1.0 mL/min, 254 nm).

Step 7: (S)-2-Chloro-4-(1-((5-methoxy-7-methyl-1H-indol-4-yl)methyl)-4-(3,3,3-trifluoropropyl)piperazin-2-yl)benzoic acid & (R)-2-Chloro-4-(1-((5-methoxy-7-methyl-1H-indol-4-yl)methyl)-4-(3,3,3-trifluoropropyl)piperazin-2-yl)benzoic acid.

To a stirred mixture of (S)-2-Chloro-4-(1-((5-methoxy-7-methyl-1H-indol-4-yl)methyl)-4-(3,3,3-trifluoropropyl)piperazin-2-yl)benzoic acid (63 mg, 0.10 mmol) and Potassium hydroxide (315 mg, 5.61 mmol) in ethanol (3 mL) and water (0.5 mL). The resulting mixture was stirred for 2 h at 60° C. The reaction mixture was acidified to pH=6 with sat. citric acid solution. The solvent was removed under vacuum. The residue was purified by reverse flash chromatography with the following conditions: column, C18 silica gel; mobile phase, MeOH in Water (10 mmol/L NH₄HCO₃), 10% to 50% gradient in 20 min; detector, UV 254 nm. to afford(S)-2-Chloro-4-(1-((5-methoxy-7-methyl-1H-indol-4-yl)methyl)-4-(3,3,3-trifluoropropyl)piperazin-2-yl)benzoic acid (39.5 mg, 77.5% yield) as a white solid. LCMS (ESI) [M+H]⁺=510.2. ¹H NMR (300 MHz, Methanol-d₄) δ 7.76-7.60 (m, 2H), 7.57-7.45 (m, 1H), 7.31 (d, J=3.1 Hz, 1H), 6.76 (s, 1H), 6.40 (d, J=3.2 Hz, 1H), 4.30 (d, J=12.7 Hz, 2H), 4.00 (d, J=12.5 Hz, 1H), 3.81 (s, 3H), 3.27-2.94 (m, 3H), 2.82-2.66 (m, 3H), 2.65-2.34 (m, 7H).

To a stirred mixture of (R)-2-Chloro-4-(1-((5-methoxy-7-methyl-1H-indol-4-yl)methyl)-4-(3,3,3-trifluoropropyl)piperazin-2-yl)benzoic acid (70 mg, 0.11 mmol) and Potassium hydroxide (315 mg, 5.61 mmol) in ethanol (3 mL) and water (0.5 mL). The resulting mixture was stirred for additional 2 h at 60° C. The reaction mixture was acidified to pH=6 with sat. citric acid solution. The solvent was removed under vacuum. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 30 min; detector, UV 254 nm. to afford (R)-2-Chloro-4-(1-((5-methoxy-7-methyl-1H-indol-4-yl)methyl)-4-(3,3,3-trifluoropropyl)piperazin-2-yl)benzoic acid (39.7 mg, 70.7% yield) as a white solid. LCMS (ESI) [M+H]⁺=510.2. ¹H NMR (300 MHz, Methanol-d₄) δ 7.76-7.61 (m, 2H), 7.51 (d, J=7.9 Hz, 1H), 7.31 (d, J=3.1 Hz, 1H), 6.76 (s, 1H), 6.40 (d, J=3.2 Hz, 1H), 4.30 (d, J=12.8 Hz, 2H), 4.01 (d, J=12.6 Hz, 1H), 3.81 (s, 3H), 3.25-2.96 (m, 3H), 2.84-2.66 (m, 2H), 2.65-2.35 (m, 7H).

### Example 15. 4-(4-(2,2-Difluoroethyl)-1-((5-methoxy-7-methyl-1H-indol-4-yl)methyl)piperazin-2-yl)-2-(((R)-1,1,1-trifluoropropan-2-yl)amino)benzoic acid

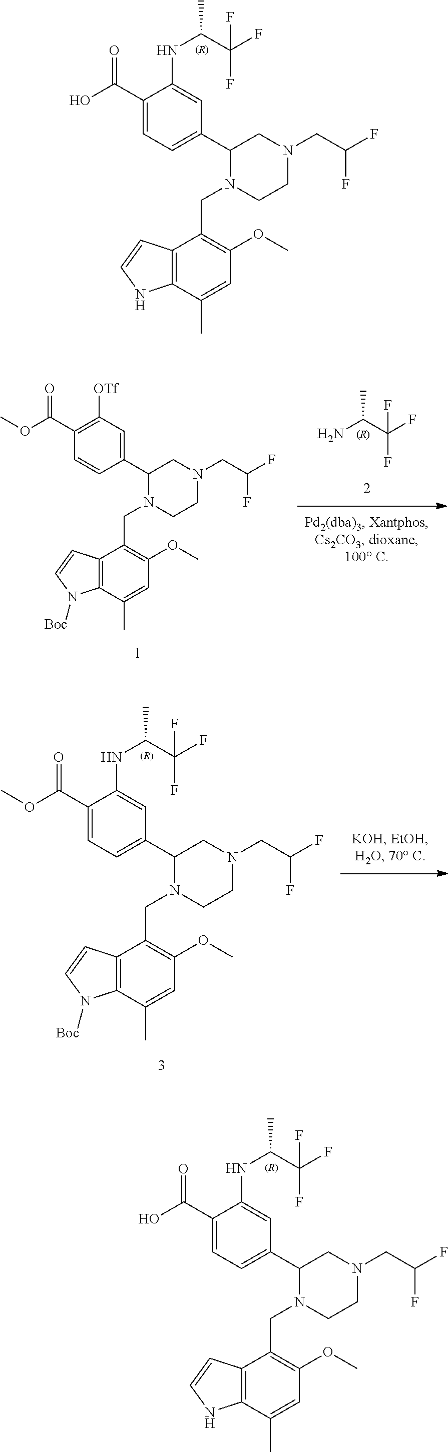

Step 1: tert-Butyl 4-((4-(2,2-difluoroethyl)-2-(4-(methoxycarbonyl)-3-(((R)-1,1,1-trifluoropropan-2-yl)amino)phenyl)piperazin-1-yl)methyl)-5-methoxy-7-methyl-1H-indole-1-carboxylate. A mixture of tert-butyl 4-((4-(2,2-difluoroethyl)-2-(4-(methoxycarbonyl)-3-(trifluoromethanesulfonyloxy)phenyl)piperazin-1-yl)methyl)-5-methoxy-7-methylindole-1-carboxylate (500 mg, 0.71 mmol), (2R)-1,1,1-trifluoropropan-2-amine (241 mg, 2.13 mmol), Pd₂(dba)₃ (130 mg, 0.14 mmol), XantPhos (164 mg, 0.28 mmol) and Cs₂CO₃ (693 mg, 2.13 mmol) in dioxane (10 mL) was stirred for overnight at 100° C. under nitrogen. The resulting mixture was diluted with ethyl acetate. The residue was washed with water for 1 time. The combined organic layer was concentrated under reduced pressure. The residue was purified by silica gel column chromatography, eluted with petroleum ether/ethyl acetate (5:1) to afford the title compound (165 mg, 34.8% yield) as a yellow solid. LCMS (ESI): [M+H]⁺=669.3.

Step 2: 4-(4-(2,2-Difluoroethyl)-1-((5-methoxy-7-methyl-1H-indol-4-yl)methyl)piperazin-2-yl)-2-(((R)-1,1,1-trifluoropropan-2-yl)amino)benzoic acid. A mixture of tert-butyl 4-((4-(2,2-difluoroethyl)-2-(4-(methoxycarbonyl)-3-(((R)-1,1,1-trifluoropropan-2-yl)amino)phenyl)piperazin-1-yl)methyl)-5-methoxy-7-methyl-1H-indole-1-carboxylate (120 mg, 0.18 mmol) and Potassium hydroxide (101 mg, 1.80 mmol) in water (0.2 mL) and Ethanol (2 mL) was stirred for 1 h at 80° C. The mixture was acidified to pH 6 with sat. citric acid. The residue was purified by reversed-phase flash chromatography with the following conditions: column, C18 silica gel; mobile phase, MeCN in Water (10 mmol/L NH₄HCO₃), 10% to 40% gradient in 30 min; detector, UV 254 nm. The solvent was concentrated under reduced pressure to afford the title compound (65.5 mg, 65.8% yield) as a white solid. LCMS (ESI): [M+H]⁺=555.2. ¹H NMR (300 MHz, DMSO-d₆) δ 13.19-12.64 (s, 1H), 10.84 (d, J=7.0 Hz, 1H), 8.18 (s, 1H), 7.92-7.85 (m, 1H), 7.28-7.20 (m, 1H), 7.17 (s, 1H), 6.97 (s, 1H), 6.67 (s, 1H), 6.50 (t, J=2.4 Hz, 1H), 6.34-5.89 (m, 1H), 4.72 (s, 1H), 3.71 (d, J=2.5 Hz, 3H), 3.61 (d, J=12.1 Hz, 1H), 3.31-3.21 (m, 2H), 2.89-2.55 (m, 5H), 2.43 (s, 3H), 2.37-2.10 (m, 3H), 1.37 (d, J=6.6 Hz, 3H).

### Example 16. 4-(4-(2,2-difluoroethyl)-1-((5-methoxy-7-methyl-1H-indol-4-yl)methyl)piperazin-2-yl)-2-(((S)-1,1,1-trifluoropropan-2-yl)amino)benzoic acid

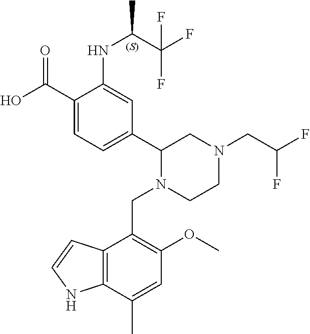

The title compound was prepared according to the Example above.

4-(4-(2,2-difluoroethyl)-1-((5-methoxy-7-methyl-1H-indol-4-yl)methyl)piperazin-2-yl)-2-(((S)-1,1,1-trifluoropropan-2-yl)amino)benzoic acid (64.8 mg, 65.1% yield) as a white solid. LCMS (ESI): [M+H]⁺=555.2. ¹H NMR (300 MHz, DMSO-d₆) δ 10.84 (d, J=7.0 Hz, 1H), 8.51 (s, 1H), 7.92-7.85 (m, 1H), 7.28-7.20 (m, 1H), 7.11 (s, 1H), 6.92 (s, 1H), 6.67 (s, 1H), 6.50 (t, J=2.4 Hz, 1H), 6.34-5.89 (m, 1H), 4.66 (s, 1H), 3.71 (d, J=2.5 Hz, 3H), 3.61 (d, J=12.1 Hz, 1H), 3.31-3.21 (m, 2H), 2.87-2.55 (m, 5H), 2.42 (s, 3H), 2.38-2.10 (m, 3H), 1.36 (d, J=6.6 Hz, 3H).

### Example 17. 2-cyano-4-((2S)-1-((5-methoxy-7-methyl-1H-indol-4-yl)methyl)-4-(3,3,3-trifluoropropyl)piperazin-2-yl)benzoic acid

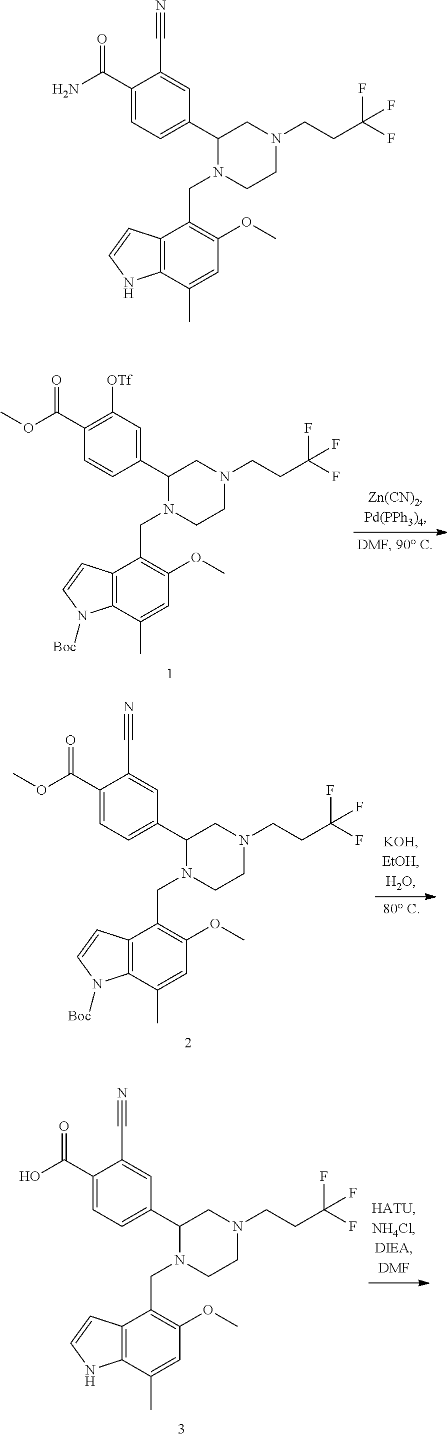 [SMILES: COc1cc(C)c2[nH]ccc2c1CN1CCN(CCC(F)(F)F)CC1c1ccc(C(N)=O)c(C#N)c1 ; COC(=O)c1ccc(C2CN(CCC(F)(F)F)CCN2Cc2c(OC)cc(C)c3c2ccn3C(=O)OC(C)(C)C)cc1OS(=O)(=O)C(F)(F)F ; COC(=O)c1ccc(C2CN(CCC(F)(F)F)CCN2Cc2c(OC)cc(C)c3c2ccn3C(=O)OC(C)(C)C)cc1C#N ; COc1cc(C)c2[nH]ccc2c1CN1CCN(CCC(F)(F)F)CC1c1ccc(C(=O)O)c(C#N)c1]

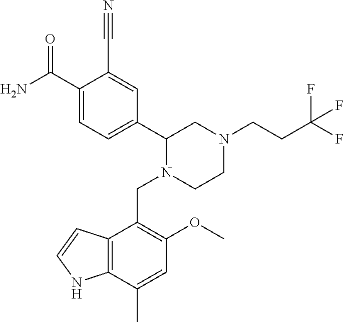 [SMILES: COc1cc(C)c2[nH]ccc2c1CN1CCN(CCC(F)(F)F)CC1c1ccc(C(N)=O)c(C#N)c1]

Step 1: tert-Butyl 4-((2-(3-cyano-4-(methoxycarbonyl)phenyl)-4-(3,3,3-trifluoropropyl)piperazin-1-yl)methyl)-5-methoxy-7-methylindole-1-carboxylate. A solution of tert-butyl 5-methoxy-4-((2-(4-(methoxycarbonyl)-3-(trifluoromethanesulfonyloxy)phenyl)-4-(3,3,3-trifluoropropyl)piperazin-1-yl)methyl)-7-methylindole-1-carboxylate (300 mg, 0.41 mmol) in DMF (3 mL) was added (96 mg, 0.81 mmol) and Pd(PPh₃)₄ (47 mg, 0.04 mmol) and stirred for 5 hours at 100° C. under nitrogen atmosphere. The solvent was removed. The residue was purified by flash with petroleum ether/ethyl acetate (4/1) to afford the title compound (200 mg, 80.0% yield) as a yellow solid. LCMS (ESI): [M+H]⁺=615.3.

Step 2: 2-Cyano-4-(1-((5-methoxy-7-methyl-1H-indol-4-yl)methyl)-4-(3,3,3-trifluoropropyl)piperazin-2-yl)benzoic acid. A solution of tert-butyl 4-((2-(3-cyano-4-(methoxycarbonyl)phenyl)-4-(3,3,3-trifluoropropyl)piperazin-1-yl)methyl)-5-methoxy-7-methylindole-1-carboxylate (200 mg, 0.32 mmol) in Ethanol (2 mL) was added Potassium hydroxide (183 mg, 3.25 mmol) in water (0.4 mL) and stirred for 2 hours at 80° C. The mixture was allowed to cool down to room temperature and was neutralized to pH=6 with sat. Citric acid. The reaction mixture was diluted with water and extracted with ethyl acetate. The organic layer was concentrated under vacuum to afford the title compound (150 mg) as crude. LCMS (ESI): [M+H]⁺=501.3.

Step 3: 2-Cyano-4-(1-((5-methoxy-7-methyl-1H-indol-4-yl)methyl)-4-(3,3,3-trifluoropropyl)piperazin-2-yl)benzamide. A solution of 2-cyano-4-(1-((5-methoxy-7-methyl-1H-indol-4-yl)methyl)-4-(3,3,3-trifluoropropyl)piperazin-2-yl)benzoic acid (150 mg, 0.30 mmol) in DMF (4 mL) was added HATU (171 mg, 0.45 mmol), NH₄Cl (48 mg, 0.90 mmol) and N,N-diisopropylethylamine (116 mg, 0.90 mmol) and stirred for 2 hours at room temperature. The reaction mixture was diluted with ethyl acetate and washed water and the organic layers were combined. The organic layer was concentrated under vacuum and purified by flash with petroleum ether/ethyl acetate (3/2) to afford the title compound (50.8 mg, 33.0% yield) as a white solid. LCMS (ESI): [M+H]⁺=500.1. ¹H NMR (300 MHz, Methanol-d₄) δ 8.10 (s, 1H), 7.91-7.85 (d, J=8.4 Hz, 1H), 7.85-7.79 (d, J=7.9 Hz, 1H), 7.29-7.18 (d, J=3.1 Hz, 1H), 6.69 (s, 1H), 6.50-6.43 (d, J=3.1 Hz, 1H), 3.85-3.72 (m, 4H), 3.57 (d, J=10.3 Hz, 1H), 3.31 (s, 1H), 2.85 (d, J=11.8 Hz, 1H), 2.89-2.81 (m, 2H), 2.70-2.56 (m, 2H), 2.49 (d, J=0.8 Hz, 3H), 2.46-2.31 (m, 3H), 2.31-2.10 (m, 2H).

### Example 18. (S)-4-(4-(2,2-Difluoroethyl)-1-((5-methoxy-7-methyl-1H-indol-4-yl)methyl)piperazin-2-yl)-2-methoxybenzoic acid & (R)-4-(4-(2,2-Difluoroethyl)-1-((5-methoxy-7-methyl-1H-indol-4-yl)methyl)piperazin-2-yl)-2-methoxybenzoic acid

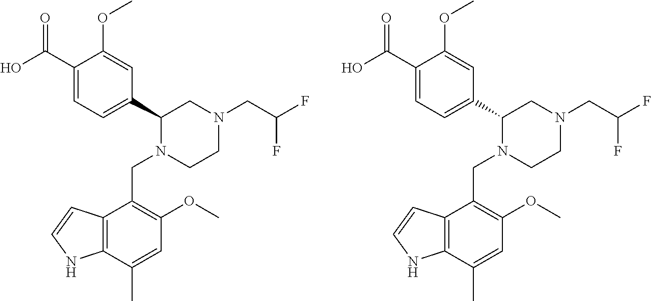 [SMILES: COc1cc([C@H]2CN(CC(F)F)CCN2Cc2c(OC)cc(C)c3[nH]ccc23)ccc1C(=O)O ; COc1cc([C@@H]2CN(CC(F)F)CCN2Cc2c(OC)cc(C)c3[nH]ccc23)ccc1C(=O)O]

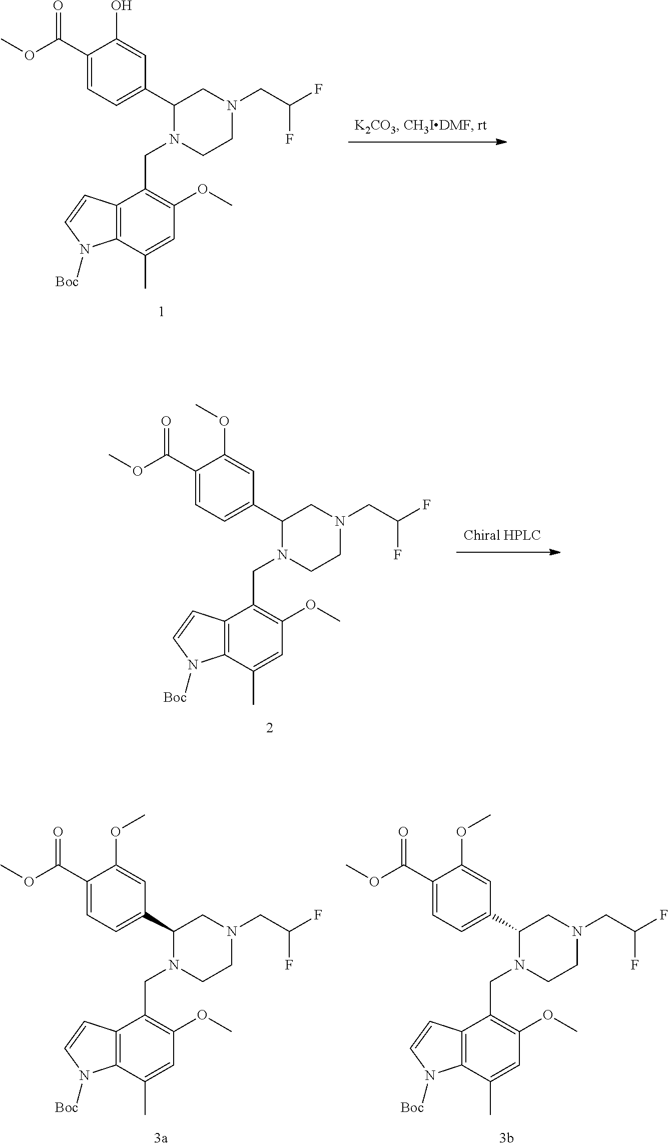 [SMILES: COC(=O)c1ccc(C2CN(CC(F)F)CCN2Cc2c(OC)cc(C)c3c2ccn3C(=O)OC(C)(C)C)cc1O ; COC(=O)c1ccc(C2CN(CC(F)F)CCN2Cc2c(OC)cc(C)c3c2ccn3C(=O)OC(C)(C)C)cc1OC ; COC(=O)c1ccc([C@H]2CN(CC(F)F)CCN2Cc2c(OC)cc(C)c3c2ccn3C(=O)OC(C)(C)C)cc1OC ; COC(=O)c1ccc([C@@H]2CN(CC(F)F)CCN2Cc2c(OC)cc(C)c3c2ccn3C(=O)OC(C)(C)C)cc1OC]

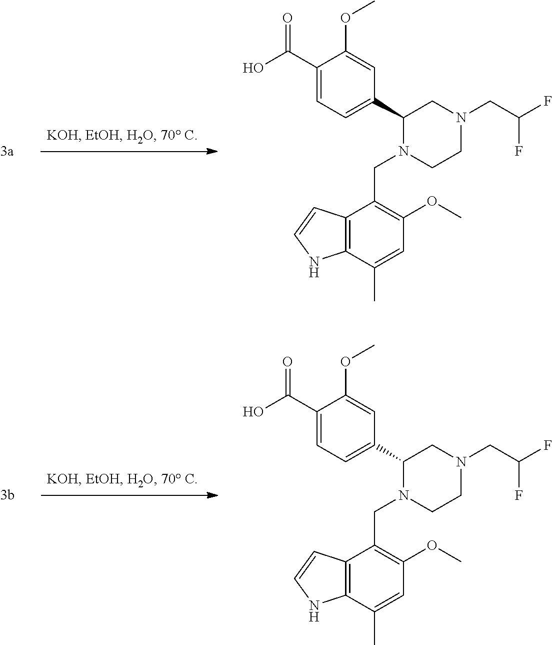

Step 1: tert-Butyl 4-((4-(2,2-difluoroethyl)-2-(3-methoxy-4-(methoxycarbonyl)phenyl)piperazin-1-yl)methyl)-5-methoxy-7-methyl-1H-indole-1-carboxylate. To a stirred mixture of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (500 mg, 0.87 mmol) and Potassium carbonate (128 mg, 1.75 mmol) in DMF (5 mL) was added Iodomethane (250 mg, 1.76 mmol). The resulting mixture was stirred for additional 2 h at room temperature. The reaction was quenched with water and extracted with ethyl acetate. The organic layer was washed with brine. The solvent was removed. The residue was purified by silica gel column chromatography, eluted with ethyl acetate/petroleum ether (1/8) to afford the title compound (260 mg, 50.7% yield) as a yellow solid. LCMS (ESI): [M+H]⁺=588.3.

Step 2: tert-Butyl(S)-4-((4-(2,2-difluoroethyl)-2-(3-methoxy-4-(methoxycarbonyl)phenyl)piperazin-1-yl)methyl)-5-methoxy-7-methyl-1H-indole-1-carboxylate & tert-Butyl (R)-4-((4-(2,2-difluoroethyl)-2-(3-methoxy-4-(methoxycarbonyl)phenyl)piperazin-1-yl)methyl)-5-methoxy-7-methyl-1H-indole-1-carboxylate.

tert-Butyl 4-((4-(2,2-difluoroethyl)-2-(3-methoxy-4-(methoxycarbonyl)phenyl)piperazin-1-yl)methyl)-5-methoxy-7-methyl-1H-indole-1-carboxylate (180 mg, 0.21 mmol) was applied for further separation by Chiral HPLC.

tert-Butyl(S)-4-((4-(2,2-difluoroethyl)-2-(3-methoxy-4-(methoxycarbonyl)phenyl)piperazin-1-yl)methyl)-5-methoxy-7-methyl-1H-indole-1-carboxylate (70 mg, 38.9% yield), LCMS (ESI) [M+H]⁺=588.2, t_{R}=2.01 min (CHIRALPAK IG-3, 4.6*50 mm, 3 μm, Hex (0.1% DEA):Ethanol=80:20, 1.0 mL/min, 254 nm).

tert-Butyl (R)-4-((4-(2,2-difluoroethyl)-2-(3-methoxy-4-(methoxycarbonyl)phenyl)piperazin-1-yl)methyl)-5-methoxy-7-methyl-1H-indole-1-carboxylate (70 mg, 38.9% yield), LCMS (ESI) [M+H]⁺=588.2, t_{R}=2.81 min (CHIRALPAK IG-3, 4.6*50 mm, 3 μm, Hex (0.1% DEA):Ethanol=80:20, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-methoxybenzoic acid & (R)-4-(4-(2,2-Difluoroethyl)-1-((5-methoxy-7-methyl-1H-indol-4-yl)methyl)piperazin-2-yl)-2-methoxybenzoic acid. To a stirred mixture of tert-Butyl(S)-4-((4-(2,2-difluoroethyl)-2-(3-methoxy-4-(methoxycarbonyl)phenyl)piperazin-1-yl)methyl)-5-methoxy-7-methyl-1H-indole-1-carboxylate (60 mg, 0.10 mmol) and Potassium hydroxide (200 mg, 3.56 mmol) in ethanol (3 mL) and water (0.6 mL) was stirred for 4 h at 70° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. to afford the title compound (37.4 mg, 77.4% yield) as a white solid. LCMS (ESI) [M+H]⁺=474.2. ¹H NMR (300 MHz, Methanol-d₄) δ 7.64 (d, J=7.7 Hz, 1H), 7.27 (d, J=3.2 Hz, 2H), 7.17 (d, J=7.8 Hz, 1H), 6.73 (s, 1H), 6.39 (d, J=3.2 Hz, 1H), 5.97 (tt, J=55.8, 4.2 Hz, 1H), 4.16 (d, J=12.3 Hz, 1H), 4.07 (d, J=10.3 Hz, 1H), 3.93 (s, 3H), 3.77 (s, 4H), 3.16 (d, J=11.9 Hz, 1H), 3.01 (s, 3H), 2.90-2.56 (m, 4H), 2.53-2.41 (m, 3H).

To a stirred mixture of tert-Butyl (R)-4-((4-(2,2-difluoroethyl)-2-(3-methoxy-4-(methoxycarbonyl)phenyl)piperazin-1-yl)methyl)-5-methoxy-7-methyl-1H-indole-1-carboxylate (60 mg, 0.10 mmol) and Potassium hydroxide (200 mg, 3.56 mmol) in ethanol (3 mL) and water (0.6 mL) was stirred for 4 h at 70° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. to afford the title compound (39.5 mg, 81.7% yield) as a white solid. LCMS (ESI) [M+H]⁺=474.2. ¹H NMR (300 MHz, Methanol-d₄) δ 7.64 (d, J=7.7 Hz, 1H), 7.27 (d, J=3.2 Hz, 2H), 7.17 (d, J=7.8 Hz, 1H), 6.73 (s, 1H), 6.39 (d, J=3.2 Hz, 1H), 5.97 (tt, J=55.8, 4.2 Hz, 1H), 4.16 (d, J=12.3 Hz, 1H), 4.07 (d, J=10.3 Hz, 1H), 3.93 (s, 3H), 3.77 (s, 4H), 3.16 (d, J=11.9 Hz, 1H), 3.01 (s, 3H), 2.90-2.56 (m, 4H), 2.53-2.41 (m, 3H).

### Example 19. (S)-2-(Cyclopropylamino)-4-(1-((5-methoxy-7-methyl-1H-indol-4-yl)methyl)-4-(3,3,3-trifluoropropyl)piperazin-2-yl)benzoic acid & (R)-2-(Cyclopropylamino)-4-(1-((5-methoxy-7-methyl-1H-indol-4-yl)methyl)-4-(3,3,3-trifluoropropyl)piperazin-2-yl)benzoic acid

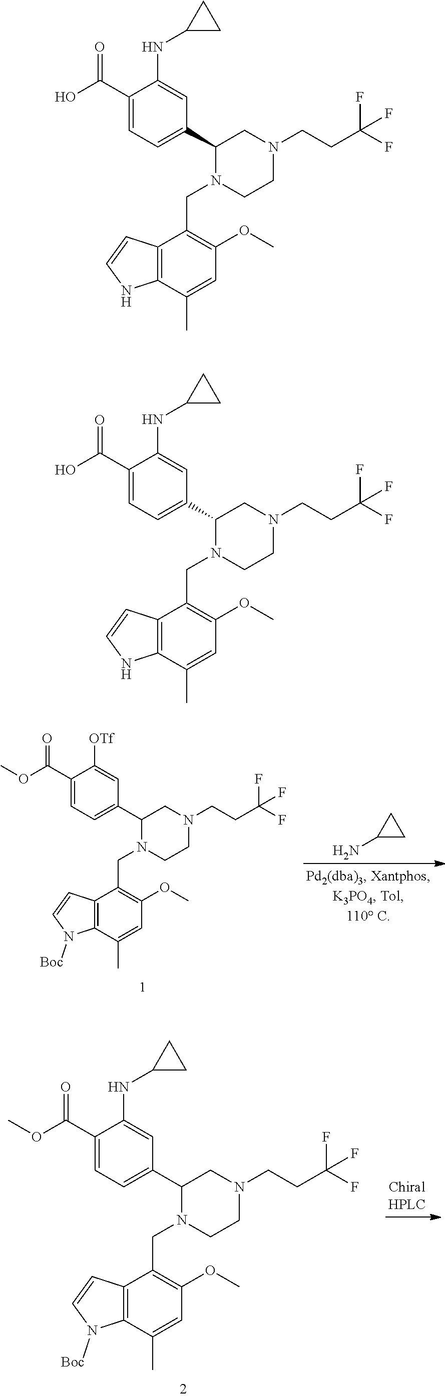

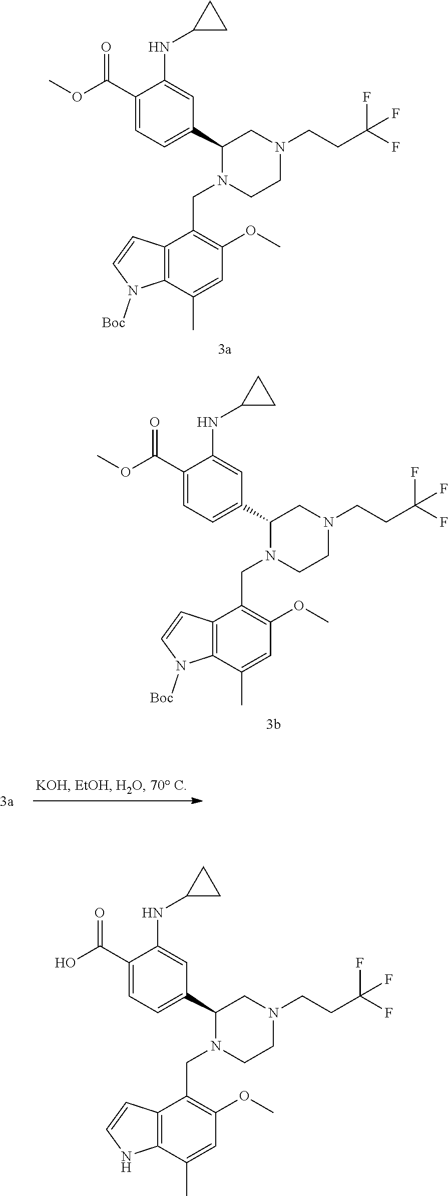 [SMILES: COC(=O)c1ccc([C@H]2CN(CCC(F)(F)F)CCN2Cc2c(OC)cc(C)c3c2ccn3C(=O)OC(C)(C)C)cc1NC1CC1 ; COC(=O)c1ccc([C@@H]2CN(CCC(F)(F)F)CCN2Cc2c(OC)cc(C)c3c2ccn3C(=O)OC(C)(C)C)cc1NC1CC1 ; COc1cc(C)c2[nH]ccc2c1CN1CCN(CCC(F)(F)F)C[C@@H]1c1ccc(C(=O)O)c(NC2CC2)c1]

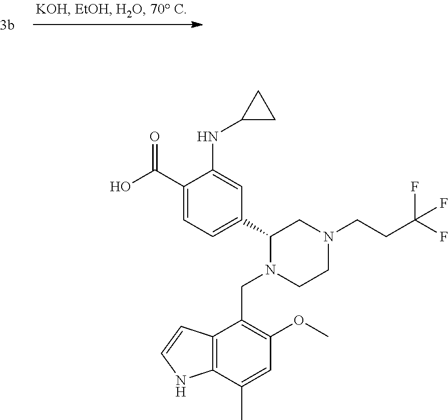 [SMILES: COc1cc(C)c2[nH]ccc2c1CN1CCN(CCC(F)(F)F)C[C@H]1c1ccc(C(=O)O)c(NC2CC2)c1]

Step 1: tert-Butyl 4-((2-(3-((cyclopropylmethyl)amino)-4-(methoxycarbonyl)phenyl)-4-(3,3,3-trifluoropropyl)piperazin-1-yl)methyl)-5-methoxy-7-methylindole-1-carboxylate. A mixture of tert-butyl 5-methoxy-4-((2-(4-(methoxycarbonyl)-3-(trifluoromethanesulfonyloxy)phenyl)-4-(3,3,3-trifluoropropyl)piperazin-1-yl)methyl)-7-methylindole-1-carboxylate (300 mg, 0.40 mmol) and cyclopropanamine (130 mg, 2.27 mmol), Pd₂(dba)₃ (80 mg, 0.09 mmol), Xantphos (94 mg, 0.16 mmol), Potassium phosphate tribasic (264 mg, 1.24 mmol) in Toluene (5 mL) was stirred for 2 h at 110° C. under nitrogen atmosphere. The resulting mixture was concentrated under reduced pressure. The residue was purified by silica gel column chromatography, eluted with petroleum ether/ethyl acetate (4:1) to afford the title compound (200 mg, 74.0% yield) as a white solid. LCMS (ESI): [M+H]⁺=645.3.

Step 2: tert-Butyl(S)-4-((2-(3-(cyclopropylamino)-4-(methoxycarbonyl)phenyl)-4-(3,3,3-trifluoropropyl)piperazin-1-yl)methyl)-5-methoxy-7-methyl-1H-indole-1-carboxylate (isomer 1, faster peak) & tert-butyl (R)-4-((2-(3-(cyclopropylamino)-4-(methoxycarbonyl)phenyl)-4-(3,3,3-trifluoropropyl)piperazin-1-yl)methyl)-5-methoxy-7-methyl-1H-indole-1-carboxylate (isomer 2, slower peak). tert-butyl 4-((2-(3-((cyclopropylmethyl)amino)-4-(methoxycarbonyl)phenyl)-4-(3,3,3-trifluoropropyl)piperazin-1-yl)methyl)-5-methoxy-7-methylindole-1-carboxylate (200 mg) was applied for separation by Chiral HPLC.

tert-Butyl(S)-4-((2-(3-(cyclopropylamino)-4-(methoxycarbonyl)phenyl)-4-(3,3,3-trifluoropropyl)piperazin-1-yl)methyl)-5-methoxy-7-methyl-1H-indole-1-carboxylate (isomer 1, faster peak) (80 mg, 40.0% yield). LCMS (ESI): [M+H]⁺=645.3. tR=1.2 min (CHIRALPAK IA-3, 4.6*50 mm, 3 μm, Hex (0.1% DEA):IPA=98:2, 1.0 mL/min, 254 nm).

tert-Butyl (R)-4-((2-(3-(cyclopropylamino)-4-(methoxycarbonyl)phenyl)-4-(3,3,3-trifluoropropyl)piperazin-1-yl)methyl)-5-methoxy-7-methyl-1H-indole-1-carboxylate (isomer 2, slower peak) (80 mg, 40.0% yield). LCMS (ESI): [M+H]⁺=645.3. tR=1.56 min (CHIRALPAK IA-3, 4.6*50 mm, 3 μm, Hex (0.1% DEA):IPA=98:2, 1.0 mL/min, 254 nm).

Step 3: (S)-2-(Cyclopropylamino)-4-(1-((5-methoxy-7-methyl-1H-indol-4-yl)methyl)-4-(3,3,3-trifluoropropyl)piperazin-2-yl)benzoic acid & (R)-2-(Cyclopropylamino)-4-(1-((5-methoxy-7-methyl-1H-indol-4-yl)methyl)-4-(3,3,3-trifluoropropyl)piperazin-2-yl)benzoic acid. To a stirred solution of tert-butyl 4-(((2S)-2-(3-(cyclopropylamino)-4-(methoxycarbonyl)phenyl)-4-(3,3,3-trifluoropropyl)piperazin-1-yl)methyl)-5-methoxy-7-methylindole-1-carboxylate (50 mg, 0.08 mmol), Potassium hydroxide (45 mg, 0.8 mmol) and water (0.5 mL) in Ethanol (1 mL) was stirred for 2 h at 70° C. The mixture was allowed to cool down to room temperature and was neutralized to pH 6 with sat. Citric acid. The residue was purified by reversed-phase flash chromatography with the following conditions: column, C18 silica gel; mobile phase, acetonitrile in Water (10 mmol/L NH₄HCO₃), 5% to 35% gradient in 20 min; detector, UV 254 nm. This resulted in(S)-2-(Cyclopropylamino)-4-(1-((5-methoxy-7-methyl-1H-indol-4-yl)methyl)-4-(3,3,3-trifluoropropyl)piperazin-2-yl)benzoic acid (23 mg, 54.2% yield) as a white solid. LCMS (ESI): [M+H]⁺=531.3. ¹H NMR (300 MHz, Methanol-d₄) δ 8.01 (d, J=8.1 Hz, 1H), 7.31 (s, 1H), 7.27 (d, J=3.1 Hz, 1H), 6.86 (d, J=8.1 Hz, 1H), 6.76 (s, 1H), 6.41 (d, J=3.2 Hz, 1H), 4.25 (d, J=12.4 Hz, 1H), 4.05 (d, J=10.4 Hz, 1H), 3.91 (d, J=12.4 Hz, 1H), 3.80 (s, 3H), 3.25 (d, J=12.3 Hz, 1H), 3.14-2.93 (m, 3H), 2.78-2.66 (m, 2H), 2.65-2.53 (m, 2H), 2.51 (s, 4H), 2.46-2.37 (m, 2H), 0.90-0.80 (m, 2H), 0.62-0.50 (m, 2H).

To a stirred solution of tert-butyl 4-(((2R)-2-(3-(cyclopropylamino)-4-(methoxycarbonyl)phenyl)-4-(3,3,3-trifluoropropyl)piperazin-1-yl)methyl)-5-methoxy-7-methylindole-1-carboxylate (50 mg, 0.08 mmol), Potassium hydroxide (45 mg, 0.8 mmol) and water (0.5 mL) in Ethanol (1 mL) was stirred for 2 h at 70° C. The mixture was allowed to cool down to room temperature and was neutralized to pH=6 with sat. Citric acid. The residue was purified by reversed-phase flash chromatography with the following conditions: column, C18 silica gel; mobile phase, acetonitrile in Water (10 mmol/L NH₄HCO₃), 5% to 35% gradient in 20 min; detector, UV 254 nm. This resulted in (R)-2-(Cyclopropylamino)-4-(1-((5-methoxy-7-methyl-1H-indol-4-yl)methyl)-4-(3,3,3-trifluoropropyl)piperazin-2-yl)benzoic acid (18.0 mg, 42.8% yield) as a white solid. LCMS (ESI): [M+H]⁺=531.3. ¹H NMR (300 MHz, Methanol-d₄) δ 8.01 (d, J=8.1 Hz, 1H), 7.31 (s, 1H), 7.27 (d, J=3.1 Hz, 1H), 6.86 (d, J=8.1 Hz, 1H), 6.76 (s, 1H), 6.41 (d, J=3.2 Hz, 1H), 4.25 (d, J=12.4 Hz, 1H), 4.05 (d, J=10.4 Hz, 1H), 3.91 (d, J=12.4 Hz, 1H), 3.80 (s, 3H), 3.25 (d, J=12.3 Hz, 1H), 3.14-2.93 (m, 3H), 2.78-2.66 (m, 2H), 2.65-2.53 (m, 2H), 2.51 (s, 4H), 2.46-2.37 (m, 2H), 0.90-0.80 (m, 2H), 0.62-0.50 (m, 2H).

### Example 20. (S)-4-(1-((5-Methoxy-7-methyl-1H-indol-4-yl)methyl)-4-(3,3,3-trifluoropropyl)piperazin-2-yl)-2-(methylamino)benzoic acid & (R)-4-(1-((5-methoxy-7-methyl-1H-indol-4-yl)methyl)-4-(3,3,3-trifluoropropyl)piperazin-2-yl)-2-(methylamino)benzoic acid

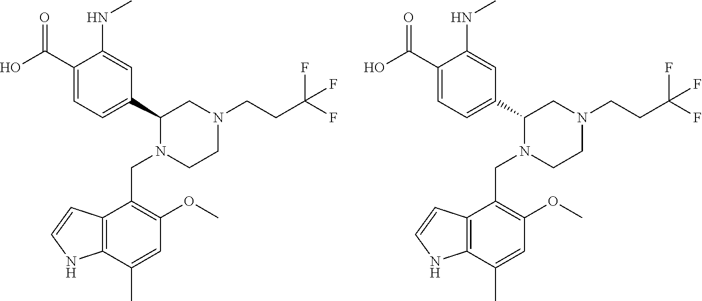 [SMILES: CNc1cc([C@H]2CN(CCC(F)(F)F)CCN2Cc2c(OC)cc(C)c3[nH]ccc23)ccc1C(=O)O ; CNc1cc([C@@H]2CN(CCC(F)(F)F)CCN2Cc2c(OC)cc(C)c3[nH]ccc23)ccc1C(=O)O]

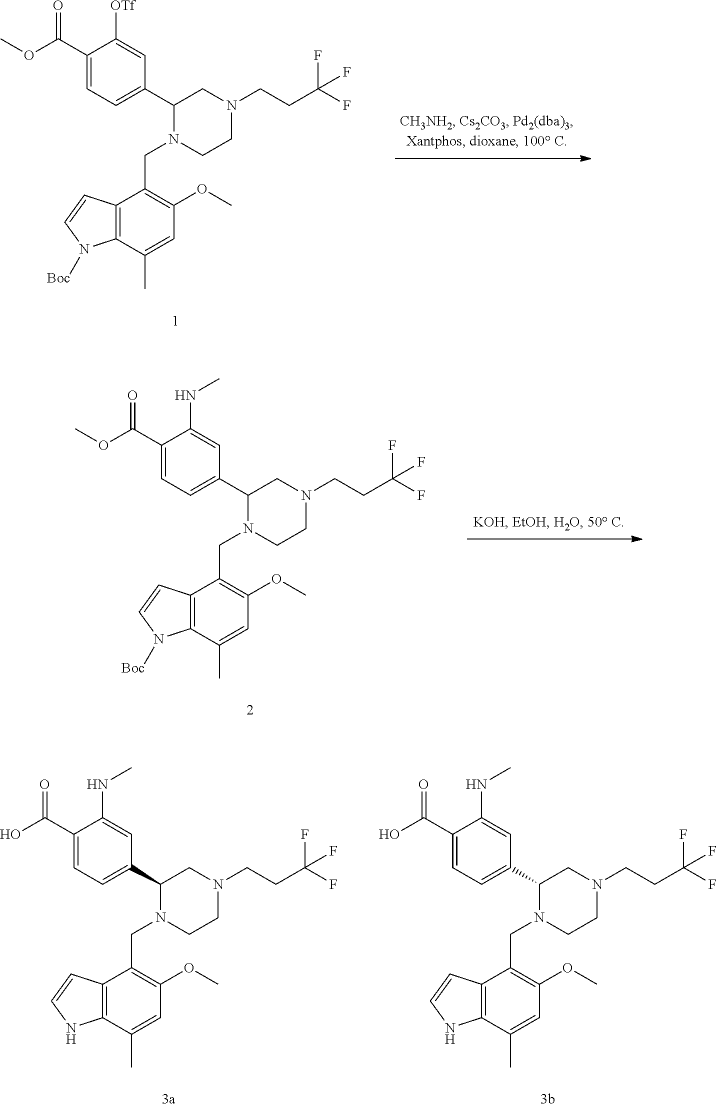 [SMILES: COC(=O)c1ccc(C2CN(CCC(F)(F)F)CCN2Cc2c(OC)cc(C)c3c2ccn3C(=O)OC(C)(C)C)cc1OS(=O)(=O)C(F)(F)F ; CNc1cc(C2CN(CCC(F)(F)F)CCN2Cc2c(OC)cc(C)c3c2ccn3C(=O)OC(C)(C)C)ccc1C(=O)OC ; CNc1cc([C@H]2CN(CCC(F)(F)F)CCN2Cc2c(OC)cc(C)c3[nH]ccc23)ccc1C(=O)O ; CNc1cc([C@@H]2CN(CCC(F)(F)F)CCN2Cc2c(OC)cc(C)c3[nH]ccc23)ccc1C(=O)O]

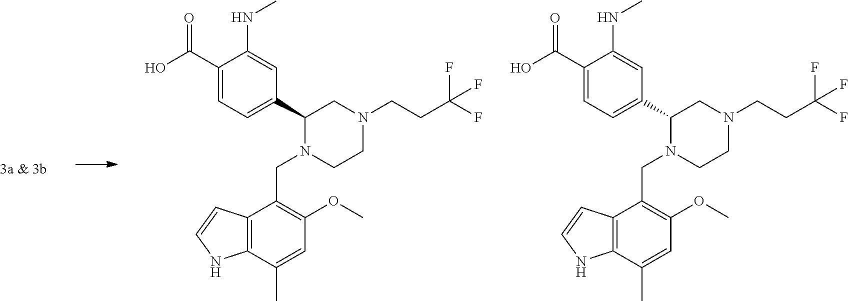 [SMILES: CNc1cc([C@H]2CN(CCC(F)(F)F)CCN2Cc2c(OC)cc(C)c3[nH]ccc23)ccc1C(=O)O ; CNc1cc([C@@H]2CN(CCC(F)(F)F)CCN2Cc2c(OC)cc(C)c3[nH]ccc23)ccc1C(=O)O]

Step 1: tert-Butyl 5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate. A mixture of tert-butyl 5-methoxy-4-((2-(4-(methoxycarbonyl)-3-(trifluoromethanesulfonyloxy)phenyl)-4-(3,3,3-trifluoropropyl)piperazin-1-yl)methyl)-7-methylindole-1-carboxylate (400 mg, 0.54 mmol), Pd₂(dba)₃ (99 mg, 0.11 mmol), XantPhos (126 mg, 0.22 mmol), Cesium carbonate (530 mg, 1.63 mmol) and 2 M CH₃NH₂ in THF (1.35 mL, 2.7 mmol) in dioxane (6 mL) was stirred for 5 h at 100° C. under N₂. The solvent was removed. The residue was purified by reversed-phase flash chromatography with the following conditions: column, C18 silica gel; mobile phase, MeCN in Water (10 mmol/L NH₄HCO₃), 70% to 100% gradient in 30 min; detector, UV 254 nm. The solvent was concentrated under reduced pressure to afford the title compound (205 mg, 61.1% yield) as a yellow solid. LCMS (ESI): [M+H]⁺=619.3.

Step 2: tert-Butyl(S)-5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (isomer 1, faster peak) & tert-butyl (R)-5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (isomer 2, slower peak).

tert-Butyl 5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (205 mg, 0.33 mmol) was applied for further separation by Chiral HPLC with the folloing condition: Column: CHIRAL ART Cellulose-SC, 2*25 cm, 5 μm; Mobile Phase A: Hex (0.5% 2M NH₃-MeOH)-HPLC, Mobile Phase B: IPA-HPLC; Flow rate: 20 mL/min; Gradient: 2% B to 2% B in 10 min; Wave Length: 220/254 nm; RT1(min): 6.566; RT2(min): 8.327; Sample Solvent: Ethanol-HPLC; Injection Volume: 0.4 mL; Number of Runs: 16.

tert-Butyl(S)-5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (isomer 1, faster peak) (75 mg, 36.6% yield). LCMS (ESI): [M+H]⁺=619.3. t_{R}=1.91 min (CHIRALPAK IC-3, 4.6*50 mm, 3 μm), Hex (0.1% DEA):IPA=98:2, 1.0 mL/min, 254 nm).

tert-Butyl (R)-5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (isomer 2, slower peak) (70 mg, 34.1% yield). LCMS (ESI): [M+H]⁺=619.3. t_{R}=2.52 min (CHIRALPAK IC-3, 4.6*50 mm 3 μm), Hex (0.1% DEA):IPA=98:2, 1.0 mL/min, 254 nm).

Step 3: (S)-4-(1-((5-Methoxy-7-methyl-1H-indol-4-yl)methyl)-4-(3,3,3-trifluoropropyl)piperazin-2-yl)-2-(methylamino)benzoic acid & (R)-4-(1-((5-methoxy-7-methyl-1H-indol-4-yl)methyl)-4-(3,3,3-trifluoropropyl)piperazin-2-yl)-2-(methylamino)benzoic acid.

A solution of tert-butyl(S)-5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (isomer 1, faster peak) (75 mg, 0.12 mmol) in ethanol (1 mL) and water (0.25 mL) was added potassium hydroxide (67 mg, 1.20 mmol) and stirred for 1 h at 80° C. The reaction mixture was acidified to pH=6 with sat. citric acid solution. The solvent was removed under vacuum. The residue was purified by reverse phase chromatography (acetonitrile 10%-40%/10 mM ammonium bicarbonate in water) to afford(S)-4-(1-((5-methoxy-7-methyl-1H-indol-4-yl)methyl)-4-(3,3,3-trifluoropropyl)piperazin-2-yl)-2-(methylamino)benzoic acid (41.9 mg, 73.4% yield) as a white solid. LCMS (ESI): [M+H]⁺=505.2. ¹H NMR (300 MHz, DMSO-d₆) δ 10.84 (s, 1H), 7.81 (d, J=8.1 Hz, 1H), 7.26 (s, 1H), 6.90 (s, 1H), 6.80 (s, 1H), 6.67 (s, 1H), 6.53 (s, 1H), 3.64 (d, J=12.0 Hz, 2H), 3.33-3.16 (m, 3H), 2.92 (s, 3H), 2.86-2.72 (m, 2H), 2.70-2.57 (m, 1H), 2.49-2.38 (m, 6H), 2.26-2.01 (m, 2H), 2.05-1.85 (m, 1H).

A solution of tert-butyl (R)-5-methoxy-4-((2-(4-(methoxycarbonyl)-3-(methylamino)phenyl)-4-(3,3,3-trifluoropropyl)piperazin-1-yl)methyl)-7-methyl-1H-indole-1-carboxylate (isomer 2, slower peak) (70 mg, 0.11 mmol) in ethanol (1 mL) and water (0.25 mL) was added potassium hydroxide (62 mg, 1.10 mmol) and stirred for 1 h at 80° C. The reaction mixture was acidified to pH=6 with sat. citric acid solution. The solvent was removed under vacuum. The residue was purified by reverse phase chromatography (acetonitrile 10%-40%/10 mM ammonium bicarbonate in water) to afford (R)-4-(1-((5-methoxy-7-methyl-1H-indol-4-yl)methyl)-4-(3,3,3-trifluoropropyl)piperazin-2-yl)-2-(methylamino)benzoic acid (43.7 mg, 76.6% yield) as a white solid. LCMS (ESI): [M+H]⁺=505.2. ¹H NMR (300 MHz, DMSO-d₆) δ 10.84 (s, 1H), 7.81 (d, J=8.1 Hz, 1H), 7.26 (s, 1H), 6.90 (s, 1H), 6.80 (s, 1H), 6.67 (s, 1H), 6.53 (s, 1H), 3.64 (d, J=12.0 Hz, 2H), 3.33-3.16 (m, 3H), 2.92 (s, 3H), 2.86-2.72 (m, 2H), 2.70-2.57 (m, 1H), 2.49-2.38 (m, 6H), 2.26-2.01 (m, 2H), 2.05-1.85 (m, 1H).

### Example 21. (S)-4-(4-(2,2-Difluoroethyl)-1-((5-methoxy-7-methyl-1H-indol-4-yl)methyl)piperazin-2-yl)-2-(2-oxopiperidin-1-yl)benzoic acid & (R)-4-(4-(2,2-difluoroethyl)-1-((5-methoxy-7-methyl-1H-indol-4-yl)methyl)piperazin-2-yl)-2-(2-oxopiperidin-1-yl)benzoic acid

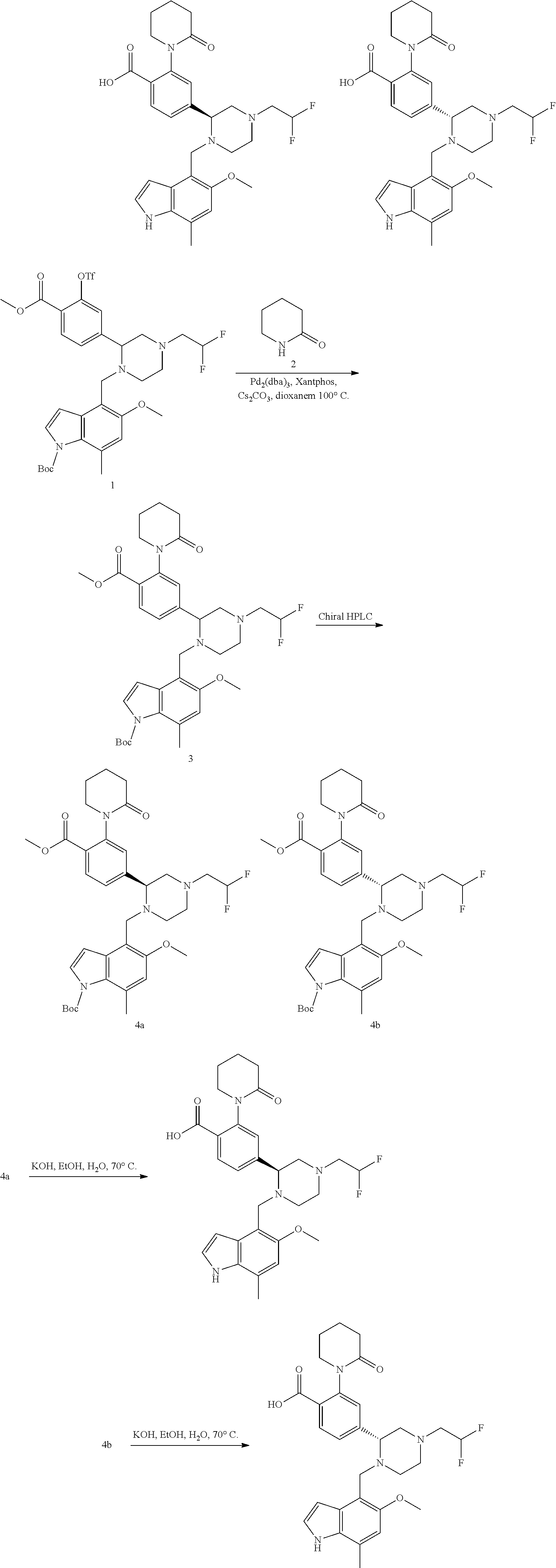

Step 1: tert-Butyl 4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate. Under nitrogen, a solution of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (500 mg, 0.70 mmol), 2-piperidinone (200 mg, 2.01 mmol), Tris(dibenzylideneacetone)dipalladium (149 mg, 0.16 mmol), XantPhos (151 mg, 0.26 mmol) and Cesium carbonate (699 mg, 2.14 mmol) in dioxane (5 mL) was stirred for 2 h at 100° C. The solvent was removed under reduced pressure. The residue was purified by silica gel column chromatography, eluted with ethyl acetate/petroleum ether (1:1) to afford the title compound (160 mg, 34.8% yield) as a yellow solid. LCMS (ESI): [M+H]⁺=655.3.

Step 2: tert-Butyl(S)-4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate (isomer 1, faster peak) & tert-butyl (R)-4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate (isomer 2, slower peak).

tert-Butyl 4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate (160 mg) was applied for further separation by Chiral HPLC.

tert-Butyl(S)-4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate (isomer 1, faster peak). LCMS (ESI): [M+H]⁺=655.3, tR=2.56 min (CHIRALPAK IE-3 (4.6*50 mm, 3 μm), Hex (0.1% DEA):Ethanol=70:30, 1.0 mL/min, 254 nm).

tert-butyl (R)-4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate (isomer 2, slower peak), LCMS (ESI): [M+H]⁺=655.3, tR=3.08 min (CHIRALPAK IE-3 (4.6*50 mm, 3 μm), Hex (0.1% DEA):Ethanol=70:30, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-(2-oxopiperidin-1-yl)benzoic acid & (R)-4-(4-(2,2-difluoroethyl)-1-((5-methoxy-7-methyl-1H-indol-4-yl)methyl)piperazin-2-yl)-2-(2-oxopiperidin-1-yl)benzoic acid. A solution of tert-butyl(S)-4-((4-(2,2-difluoroethyl)-2-(4-(methoxycarbonyl)-3-(2-oxopiperidin-1-yl)phenyl)piperazin-1-yl)methyl)-5-methoxy-7-methyl-1H-indole-1-carboxylate (78 mg, 0.11 mmol) in ethanol (3 mL) and water water (0.5 mL) was added Potassium hydroxide (200 mg, 3.56 mmol) and stirred for 2 h at 70° C. The mixture was acidified to pH=6 with sat. citric acid solution. The solvent was removed under vacuum. The residue was purified by reversed-phase flash chromatography to afford the title compound.

(S)-4-(4-(2,2-Difluoroethyl)-1-((5-methoxy-7-methyl-1H-indol-4-yl)methyl)piperazin-2-yl)-2-(2-oxopiperidin-1-yl)benzoic acid (22.4 mg, 34.7% yield) as a white solid. LCMS (ESI) [M+H]⁺=541.3, ¹H NMR (300 MHz, Methanol-d₄) § 7.97 (d, J=8.0 Hz, 1H), 7.60 (d, J=8.0 Hz, 1H), 7.44 (s, 1H), 7.23 (d, J=3.1 Hz, 1H), 6.71 (s, 1H), 6.39 (d, J=3.1 Hz, 1H), 6.21-5.70 (m, 1H), 4.08 (d, J=12.2 Hz, 1H), 3.92 (d, J=10.2 Hz, 1H), 3.78 (s, 3H), 3.75-3.64 (m, 3H), 3.08-2.96 (m, 2H), 2.93-2.90 (m, 1H), 2.85-2.69 (m, 3H), 2.61-2.46 (s, 7H), 2.01-1.95 (m, 4H).

(R)-4-(4-(2,2-Difluoroethyl)-1-((5-methoxy-7-methyl-1H-indol-4-yl)methyl)piperazin-2-yl)-2-(2-oxopiperidin-1-yl)benzoic acid was prepare according to above. (R)-4-(4-(2,2-Difluoroethyl)-1-((5-methoxy-7-methyl-1H-indol-4-yl)methyl)piperazin-2-yl)-2-(2-oxopiperidin-1-yl)benzoic acid (26.3 mg, 42.3% yield) as a white solid. LCMS (ESI) [M+H]⁺=541.3, ¹H NMR (300 MHz, Methanol-d₄) δ 7.97 (d, J=8.0 Hz, 1H), 7.60 (d, J=8.0 Hz, 1H), 7.44 (s, 1H), 7.23 (d, J=3.1 Hz, 1H), 6.71 (s, 1H), 6.39 (d, J=3.1 Hz, 1H), 6.21-5.70 (m, 1H), 4.08 (d, J=12.2 Hz, 1H), 3.92 (d, J=10.2 Hz, 1H), 3.78 (s, 3H), 3.75-3.64 (m, 3H), 3.08-2.96 (m, 2H), 2.93-2.90 (m, 1H), 2.85-2.69 (m, 3H), 2.61-2.46 (s, 7H), 2.01-1.95 (m, 4H).

### Example 22. 2-Methoxy-4-((2S)-1-((5-methoxy-7-methyl-1H-indol-4-yl)methyl)-4-(3,3,3-trifluoropropyl)piperazin-2-yl)benzoic acid & 2-methoxy-4-((2R)-1-((5-methoxy-7-methyl-1H-indol-4-yl)methyl)-4-(3,3,3-trifluoropropyl)piperazin-2-yl)benzoic acid

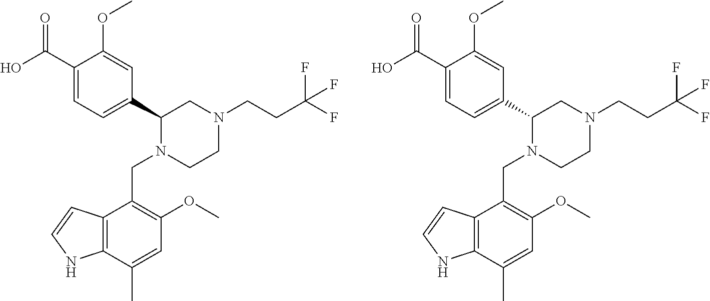 [SMILES: COc1cc([C@H]2CN(CCC(F)(F)F)CCN2Cc2c(OC)cc(C)c3[nH]ccc23)ccc1C(=O)O ; COc1cc([C@@H]2CN(CCC(F)(F)F)CCN2Cc2c(OC)cc(C)c3[nH]ccc23)ccc1C(=O)O]

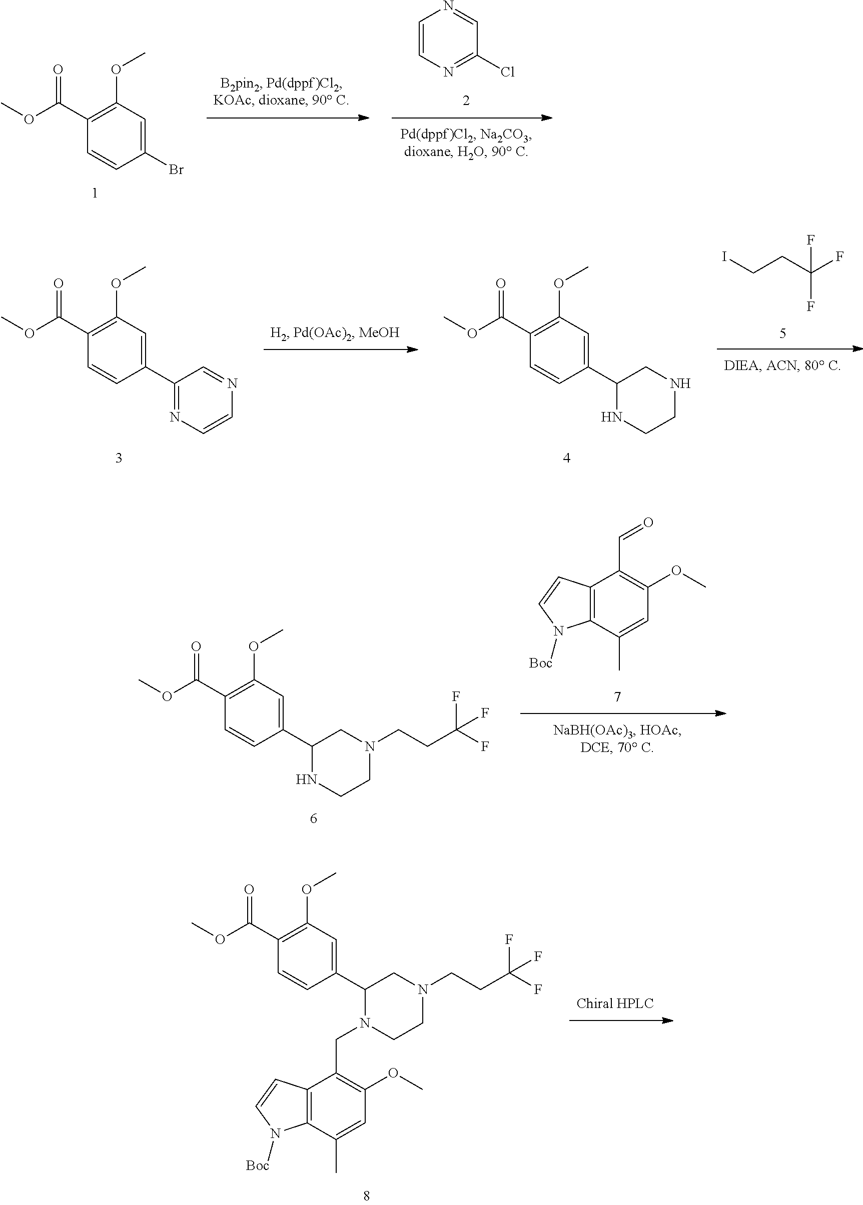

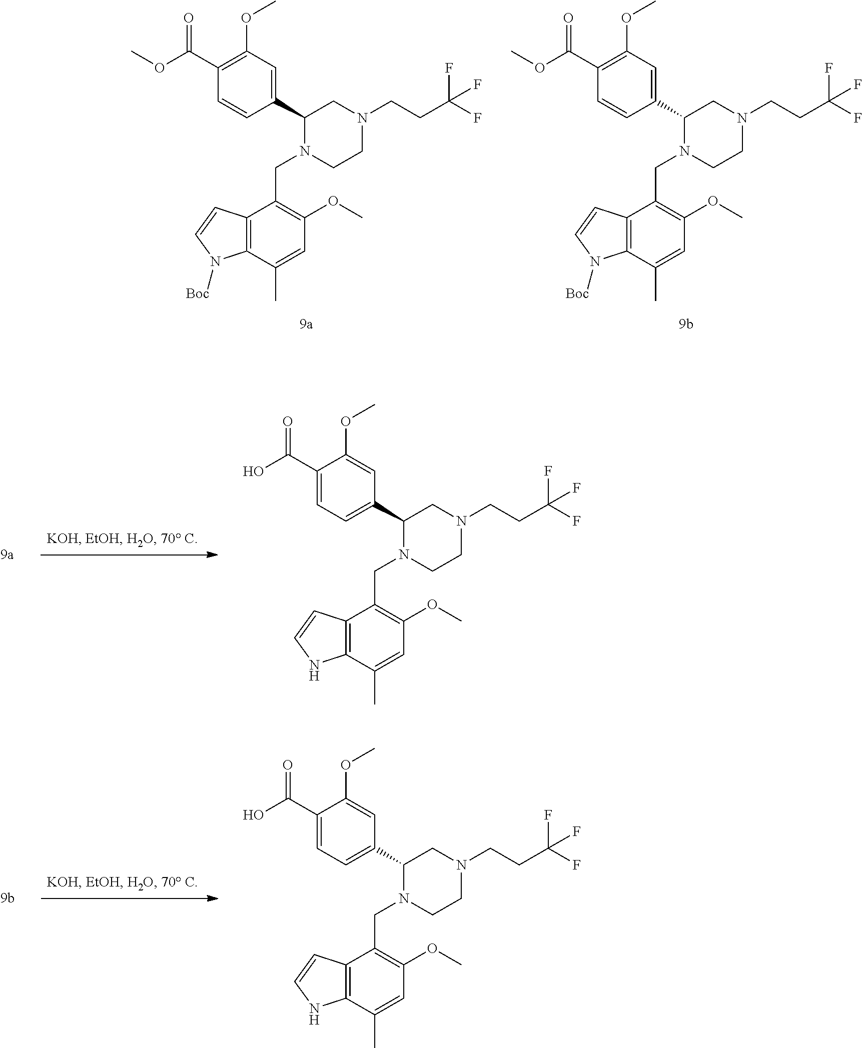

Step 1: Methyl 2-methoxy-4-(pyrazin-2-yl)benzoate. To a mixture of methyl 4-bromo-2-methoxybenzoate (3.0 g, 12.24 mmol) and bis(pinacolato)diboron (4.7 g, 18.51 mmol) in dioxane (50 mL) were added KOAc (3.6 g, 36.68 mmol) and Pd(dppf)Cl₂ (896 mg, 1.23 mmol). After stirring for 4 h at 90° C. under a nitrogen atmosphere, the resulting mixture was allowed to cool down to room temperature.

To the above mixture was added 2-chloropyrazine (2.9 g, 25.26 mmol), Sodium carbonate (4.0 g, 37.71 mmol), water (10 mL) and Pd(dppf)Cl₂ (1.4 g, 1.85 mmol). After stirring for 2 h at 90° C. under a nitrogen atmosphere, the resulting mixture was extracted with dichloromethane. The combined organic layers were washed with water, dried over anhydrous sodium sulfate. After filtration, the filtrate was concentrated under reduced pressure. The residue was purified by silica gel column chromatography, eluted with petroleum ether/ethyl acetate (2:1) to afford the title compound (2.8 g, 93.0% yield) as a yellow solid. LCMS (ESI): [M+H]⁺=245.2.

Step 2: Methyl 2-methoxy-4-(piperazin-2-yl)benzoate. To a mixture of methyl 2-methoxy-4-(pyrazin-2-yl)benzoate (2.8 g, 11.46 mmol) and Pd(OAc)₂ (305 mg, 1.36 mmol) in MeOH (40 mL) was stirred for 5 h at room temperature under hydrogen atmosphere. The resulting mixture was filtered, the filter cake was washed with MeOH. The filtrate was concentrated under reduced pressure to afford the title compound (4.3 g, 97.9% yield) as a yellow oil. LCMS (ESI): [M+H]⁺=251.3.

Step 3: Methyl 2-methoxy-4-(4-(3,3,3-trifluoropropyl)piperazin-2-yl)benzoate. To a mixture of methyl 2-methoxy-4-(piperazin-2-yl)benzoate (2.0 g, 7.99 mmol), N,N-diisopropylethylamine (3.1 g, 23.99 mmol) and 1,1,1-trifluoro-3-iodopropane (1.4 g, 6.25 mmol) in acetonitrile (40 mL) was stirred for overnight at 70° C. The resulting mixture was concentrated under reduced pressure. The residue was purified by reverse phase flash with the following conditions (acetonitrile, 5-50%/10 mM NH₄HCO₃ in water) to afford the title compound (880 mg, 31.8% yield) as a brown oil. LCMS (ESI): [M+H]⁺=347.2.

Step 4: tert-Butyl 5-methoxy-4-((2-(3-methoxy-4-(methoxycarbonyl)phenyl)-4-(3,3,3-trifluoropropyl)piperazin-1-yl)methyl)-7-methylindole-1-carboxylate. To a stirred mixture of methyl 2-methoxy-4-(4-(3,3,3-trifluoropropyl)piperazin-2-yl)benzoate (300 mg, 0.87 mmol), tert-butyl 4-formyl-5-methoxy-7-methylindole-1-carboxylate (300 mg, 1.04 mmol) and HOAc (180 mg, 2.99 mmol) in 1,2-dichloroethane (4 mL) was added Sodium triacetoxyborohydride (360 mg, 1.70 mmol) in portions at room temperature. The resulting mixture was stirred for overnight at 70° C. The resulting mixture was extracted with ethyl acetate. The combined organic layers were washed with water, dried over anhydrous sodium sulfate. After filtration, the filtrate was concentrated under reduced pressure. The residue was purified by silica gel column chromatography, eluted with petroleum ether/ethyl acetate (2:1) to afford the title compound (220 mg, 41.0% yield) as a white solid. LCMS (ESI): [M+H]⁺=620.3.

Step 5: tert-Butyl 5-methoxy-4-(((2S)-2-(3-methoxy-4-(methoxycarbonyl)phenyl)-4-(3,3,3-trifluoropropyl)piperazin-1-yl)methyl)-7-methylindole-1-carboxylate (isomer 1, faster peak) & tert-butyl 5-methoxy-4-(((2R)-2-(3-methoxy-4-(methoxycarbonyl)phenyl)-4-(3,3,3-trifluoropropyl)piperazin-1-yl)methyl)-7-methylindole-1-carboxylate (isomer 2, slower peak).

The tert-butyl 5-methoxy-4-((2-(3-methoxy-4-(methoxycarbonyl)phenyl)-4-(3,3,3-trifluoropropyl)piperazin-1-yl)methyl)-7-methylindole-1-carboxylate (240 mg) was applied for further separation by Chiral HPLC.

tert-Butyl 5-methoxy-4-(((2S)-2-(3-methoxy-4-(methoxycarbonyl)phenyl)-4-(3,3,3-trifluoropropyl)piperazin-1-yl)methyl)-7-methylindole-1-carboxylate (isomer 1, faster peak) (110 mg, 45.8% yield) as a white solid. LCMS (ESI): [M+H]⁺=620.3. t_{R}=3.30 min (CHIRALPAK ID-3, 4.6*50 mm, 3 μm, Hex (0.1% DEA):IPA=93:7, 1.0 mL/min, 254 nm).

tert-Butyl 5-methoxy-4-(((2R)-2-(3-methoxy-4-(methoxycarbonyl)phenyl)-4-(3,3,3-trifluoropropyl)piperazin-1-yl)methyl)-7-methylindole-1-carboxylate (isomer 2, slower peak) (110 mg, 45.8% yield) as a white solid. LCMS (ESI): [M+H]⁺=620.3. t_{R}=4.10 min (CHIRALPAK ID-3, 4.6*50 mm, 3 μm, Hex (0.1% DEA):IPA=93:7, 1.0 mL/min, 254 nm).

Step 6:2-Methoxy-4-((2S)-1-((5-methoxy-7-methyl-1H-indol-4-yl)methyl)-4-(3,3,3-trifluoropropyl)piperazin-2-yl)benzoic acid. To a mixture of tert-butyl 5-methoxy-4-(((2S)-2-(3-methoxy-4-(methoxycarbonyl)phenyl)-4-(3,3,3-trifluoropropyl)piperazin-1-yl)methyl)-7-methylindole-1-carboxylate (100 mg, 0.16 mmol) and Potassium hydroxide (95 mg, 1.69 mmol) in water (1 mL) and Ethanol (2 mL) was stirred for 2 h at 60° C. The mixture was acidified to pH=6 with sat. citric acid. The solvent was removed. The residue was purified by reverse phase flash with the following conditions (acetonitrile, 5-27%/10 mM NH₄HCO₃ in water) to afford the title compound (62.1 mg, 75.9% yield) as a white solid. LCMS (ESI): [M+H]⁺=506.2; ¹H NMR (300 MHz, DMSO-d₆) δ 12.05 (s, 1H), 10.99-10.72 (m, 1H), 7.66 (d, J=7.8 Hz, 1H), 7.41-7.06 (m, 3H), 6.66 (s, 1H), 6.56-6.40 (m, 1H), 3.87 (s, 3H), 3.70 (s, 3H), 3.61 (d, J=11.9 Hz, 1H), 3.45-3.36 (m, 1H), 3.30-3.18 (m, 2H), 2.89-2.57 (m, 3H), 2.48-2.30 (m, 6H), 2.28-1.28 (m, 3H).

Step 7: 2-Methoxy-4-((2R)-1-((5-methoxy-7-methyl-1H-indol-4-yl)methyl)-4-(3,3,3-trifluoropropyl)piperazin-2-yl)benzoic acid. To a mixture of tert-butyl 5-methoxy-4-(((2R)-2-(3-methoxy-4-(methoxycarbonyl)phenyl)-4-(3,3,3-trifluoropropyl)piperazin-1-yl)methyl)-7-methylindole-1-carboxylate (100 mg, 0.16 mmol) and Potassium hydroxide (95 mg, 1.69 mmol) in water (1 mL) and Ethanol (2 mL) was stirred for 2 h at 60° C. The mixture was acidified to pH=6 with sat. citric acid. The solvent was removed. The residue was purified by reverse phase flash with the following conditions (acetonitrile, 5-27%/10 mM NH₄HCO₃ in water) to afford the title compound (55.5 mg, 67.9% yield) as a white solid. LCMS (ESI): [M+H]⁺=506.2; ¹H NMR (300 MHz, DMSO-d₆) δ 11.81 (s, 1H), 10.95-10.73 (m, 1H), 7.65 (d, J=7.8 Hz, 1H), 7.37-7.09 (m, 3H), 6.66 (s, 1H), 6.55-6.37 (m, 1H), 3.86 (s, 3H), 3.70 (s, 3H), 3.61 (d, J=11.9 Hz, 1H), 3.44-3.35 (m, 1H), 3.31-3.17 (m, 2H), 2.92-2.58 (m, 3H), 2.49-2.30 (m, 6H), 2.29-1.89 (m, 3H).

### Example 23. (S)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid & (R)-5-(4-(2,2-difluoroethyl)-1-((5-methoxy-7-methyl-1H-indol-4-yl)methyl)piperazin-2-yl)-1,2,3,4-tetrahydroquinoline-8-carboxylic acid

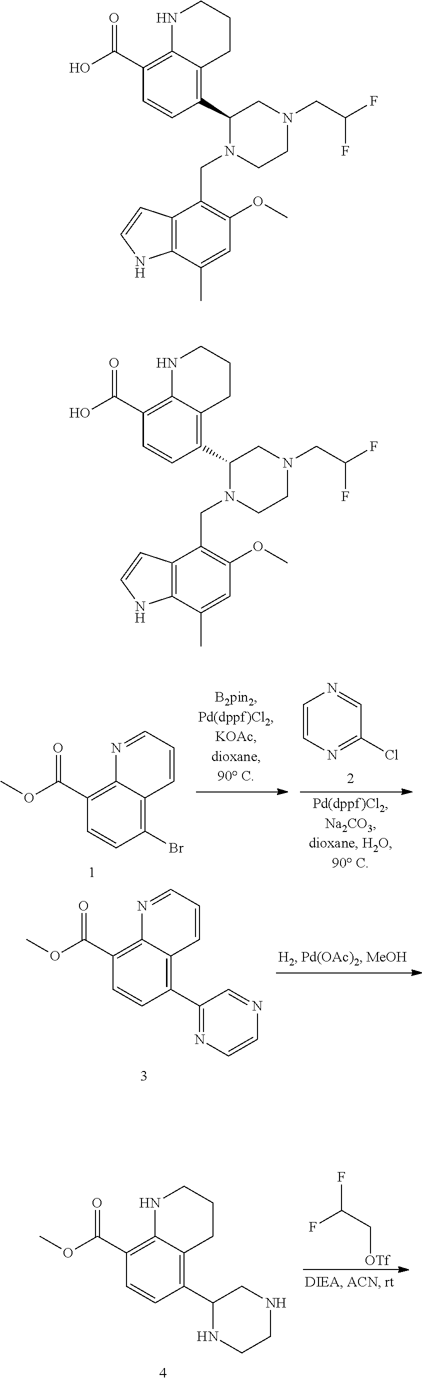

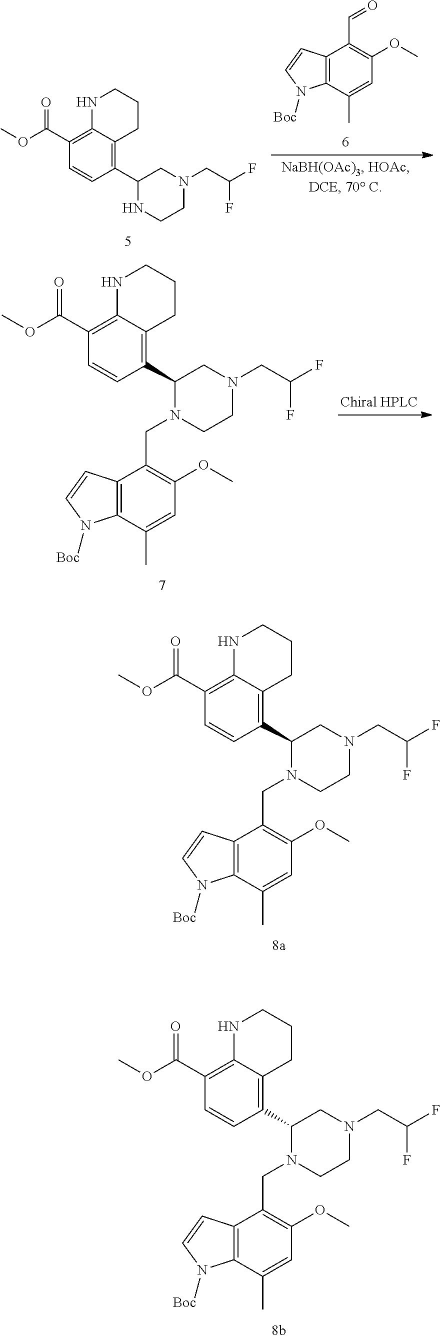

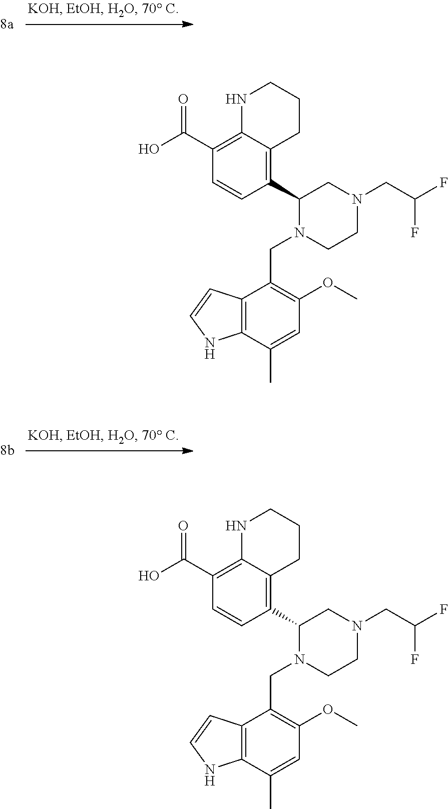 [SMILES: COc1cc(C)c2[nH]ccc2c1CN1CCN(CC(F)F)C[C@@H]1c1ccc(C(=O)O)c2c1CCCN2 ; COc1cc(C)c2[nH]ccc2c1CN1CCN(CC(F)F)C[C@H]1c1ccc(C(=O)O)c2c1CCCN2]

Step 1: Methyl 5-(pyrazin-2-yl) quinoline-8-carboxylate. A mixture of methyl 5-bromoquinoline-8-carboxylate (2 g, 7.51 mmol) and bis(pinacolato)diboron (2.01 g, 7.9 mmol), Pd(dppf)Cl₂ (0.55 g, 0.75 mmol), KOAc (1.84 g, 18.75 mmol) in dioxane (20 mL) was stirred for 1 h at 90° C. under nitrogen atmosphere. To the above mixture was added 2-chloropyrazine (1.04 g, 9.08 mmol), Pd(dppf)Cl₂ (0.55 g, 0.75 mmol), Sodium carbonate (1.6 g, 15.10 mmol) and water (2 mL). The mixture was stirred for 1 h at 90° C. under nitrogen atmosphere. The reaction was quenched with water and extracted with ethyl acetate. The solvent was removed under vacuum. The residue was purified by silica gel column chromatography, eluted with petroleum ether/ethyl acetate (1:1) to afford the title compound (1.53 g, 90% yield) as a yellow solid. LCMS (ESI): [M+H]⁺=266.1.

Step 2: Methyl 5-(piperazin-2-yl)-1,2,3,4-tetrahydroquinoline-8-carboxylate. A mixture of methyl 5-(pyrazin-2-yl) quinoline-8-carboxylate (1.53 g, 5.76 mmol) and Palladium (II) Acetate (0.65 g, 2.88 mmol) in acetic acid (20 mL) was stirred for overnight at room temperature under hydrogen. The solids were filtered out. The filtrate was concentrated under reduced pressure. The residue was purified by reverse phase flash to afford the title compound (500 mg, 80% yield) as a brown solid. LCMS (ESI): [M+H]⁺=276.2.

Step 3: Methyl 5-(4-(2,2-difluoroethyl)piperazin-2-yl)-1,2,3,4-tetrahydroquinoline-8-carboxylate. To a stirred solution of methyl 5-(piperazin-2-yl)-1,2,3,4-tetrahydroquinoline-8-carboxylate (423 mg, 1.54 mmol) and N,N-diisopropylethylamine (794 mg, 6.14 mmol) in ACN (20 mL) was added 2,2-difluoroethyl trifluoromethanesulfonate (263 mg, 1.23 mmol) dropwise in portions at 0° C. The resulting mixture was concentrated under vacuum. The residue was purified by reversed-phase flash chromatography to afford the title compound (260 mg, 90% yield) as a yellow oil. LCMS (ESI): [M+H]⁺=340.2.

Step 4: Methyl 5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate.

To a mixture of Methyl 5-(4-(2,2-difluoroethyl)piperazin-2-yl)-1,2,3,4-tetrahydroquinoline-8-carboxylate (260 mg, 0.77 mmol), tert-butyl 4-formyl-5-methoxy-7-methylindole-1-carboxylate (274 mg, 0.92 mmol), acetic acid (138 mg, 2.30 mmol) and Sodium triacetoxyborohydride (488 mg, 2.30 mmol) in dichloroethane (7 mL) was stirred for 6 h at 70° C. The solvent was removed under reduced pressure. The residue was purified by silica gel column chromatography, eluted with petroleum ether/ethyl acetate (3:1) to afford the title compound (200 mg, 95% yield) as a yellow oil. LCMS (ESI): [M+H]⁺=613.3.

Step 5: Methyl(S)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 1, faster peak) & Methyl (R)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 2, slower peak).

Methyl 5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (240 mg) was applied for further separation by Chiral HPLC.

Methyl(S)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 1, faster peak) (100 mg, 41.7% yield). LCMS (ESI): [M+H]⁺=613.3. tR=1.57 min (CHIRALPAK IH-3, 4.6*50 mm 3 μm), Hex (0.1% DEA):Ethanol=98:2, 1.0 mL/min, 254 nm).

Methyl (R)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 2, slower peak) (100 mg, 41.7% yield). LCMS (ESI): [M+H]⁺=613.3. tR=1.90 min (CHIRALPAK IH-3, 4.6*50 mm 3 μm), Hex (0.1% DEA):Ethanol=98:2, 1.0 mL/min, 254 nm).

Step 6: (S)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid & (R)-5-(4-(2,2-difluoroethyl)-1-((5-methoxy-7-methyl-1H-indol-4-yl)methyl)piperazin-2-yl)-1,2,3,4-tetrahydroquinoline-8-carboxylic acid.

A mixture of Methyl(S)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 1, faster peak) (100 mg, 0.16 mmol) and Potassium hydroxide (92 mg, 1.64 mmol) in water (0.2 mL) and ethanol (1 mL) was stirred for 4 h at 70° C. The mixture was adjusted to pH=6 with sta. citric acid. The solvent was removed. The residue was purified by reversed-phase flash chromatography. This resulted in (S)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid (31.5 mg, 38.7% yield) as a white solid. LCMS (ESI): [M+H]⁺=499.2. ¹H NMR (300 MHz, DMSO-d₆) δ 10.83 (s, 1H), 7.69 (d, J=8.4 Hz, 1H), 7.26 (t, J=2.7 Hz, 1H), 7.01 (d, J=8.5 Hz, 1H), 6.67 (s, 1H), 6.50 (t, J=2.4 Hz, 1H), 6.13 (t, J=4.3 Hz, 1H), 3.72-3.67 (m, 3H), 3.54 (d, J=9.6 Hz, 1H), 3.33-3.30 (m, 3H), 3.03-2.89 (m, 1H), 2.87-2.70 (m, 1H), 2.59-2.46 (m, 6H), 2.43 (s, 3H), 2.34-2.07 (m, 3H), 2.07-1.73 (m, 2H).

A mixture of Methyl (R)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 2, slower peak) (100 mg, 0.16 mmol) and Potassium hydroxide (92 mg, 1.64 mmol) in water (0.2 mL) and ethanol (1 mL) was stirred for 4 h at 70° C. The mixture was adjusted to pH=6 with sta. citric acid. The solvent was removed. The residue was purified by reversed-phase flash chromatography. This resulted in (R)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid (35.6 mg, 44.3% yield) as a white solid. LCMS (ESI): [M+H]⁺=499.2. ¹H NMR (300 MHz, DMSO-d₆) δ 10.83 (s, 1H), 7.69 (d, J=8.4 Hz, 1H), 7.26 (t, J=2.7 Hz, 1H), 7.01 (d, J=8.5 Hz, 1H), 6.67 (s, 1H), 6.50 (t, J=2.4 Hz, 1H), 6.13 (t, J=4.3 Hz, 1H), 3.72-3.67 (m, 3H), 3.54 (d, J=9.6 Hz, 1H), 3.33-3.30 (m, 3H), 3.03-2.89 (m, 1H), 2.87-2.70 (m, 1H), 2.59-2.46 (m, 6H), 2.43 (s, 3H), 2.34-2.07 (m, 3H), 2.07-1.73 (m, 2H).

### Example 24. (S)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid & (R)-5-(4-(2,2-difluoroethyl)-1-((5-methoxy-7-methyl-1H-indol-4-yl)methyl)piperazin-2-yl)-1,2,3,4-tetrahydroquinoline-8-carboxylic acid

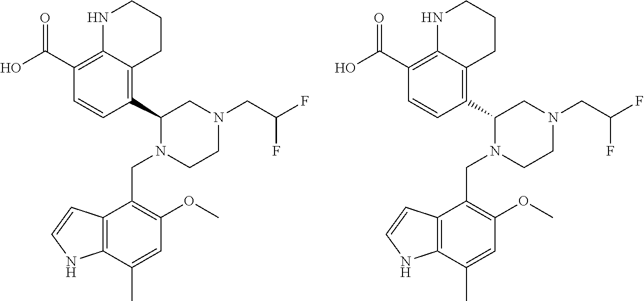 [SMILES: COc1cc(C)c2[nH]ccc2c1CN1CCN(CC(F)F)C[C@@H]1c1ccc(C(=O)O)c2c1CCCN2 ; COc1cc(C)c2[nH]ccc2c1CN1CCN(CC(F)F)C[C@H]1c1ccc(C(=O)O)c2c1CCCN2]

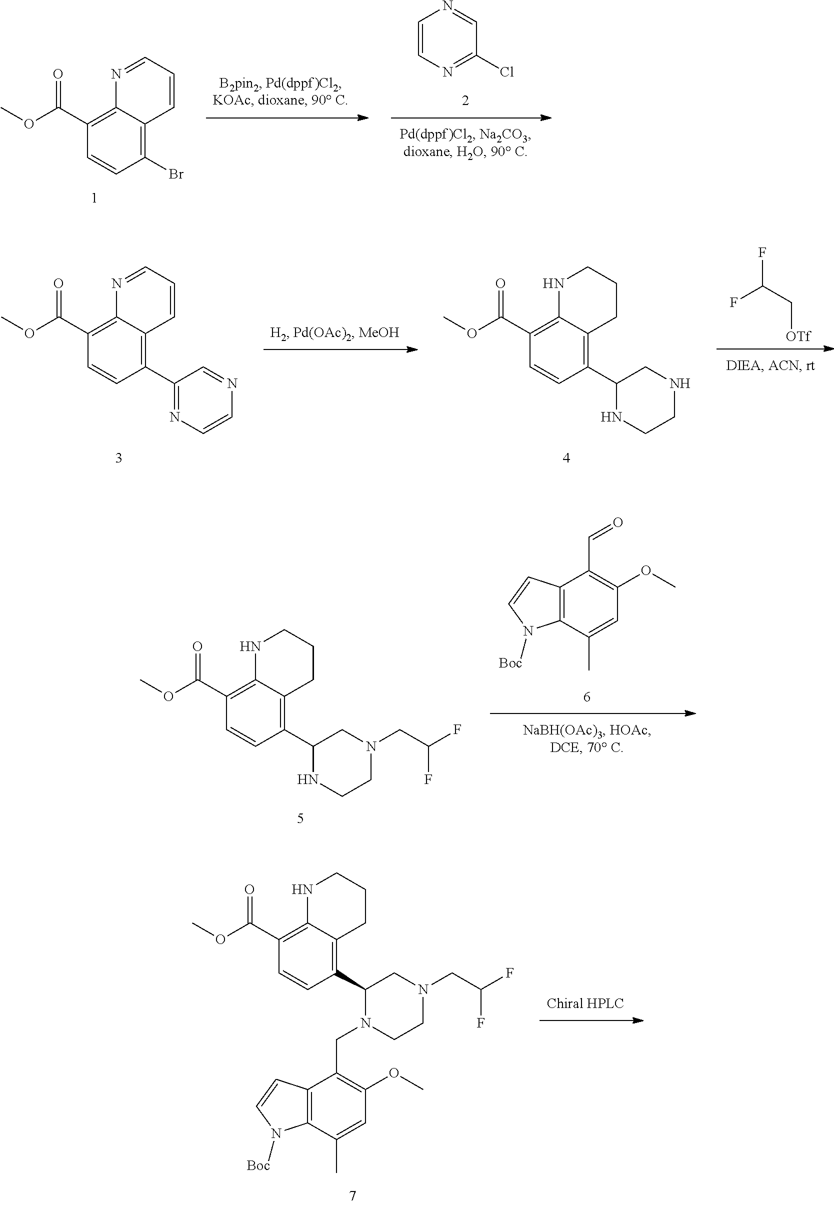

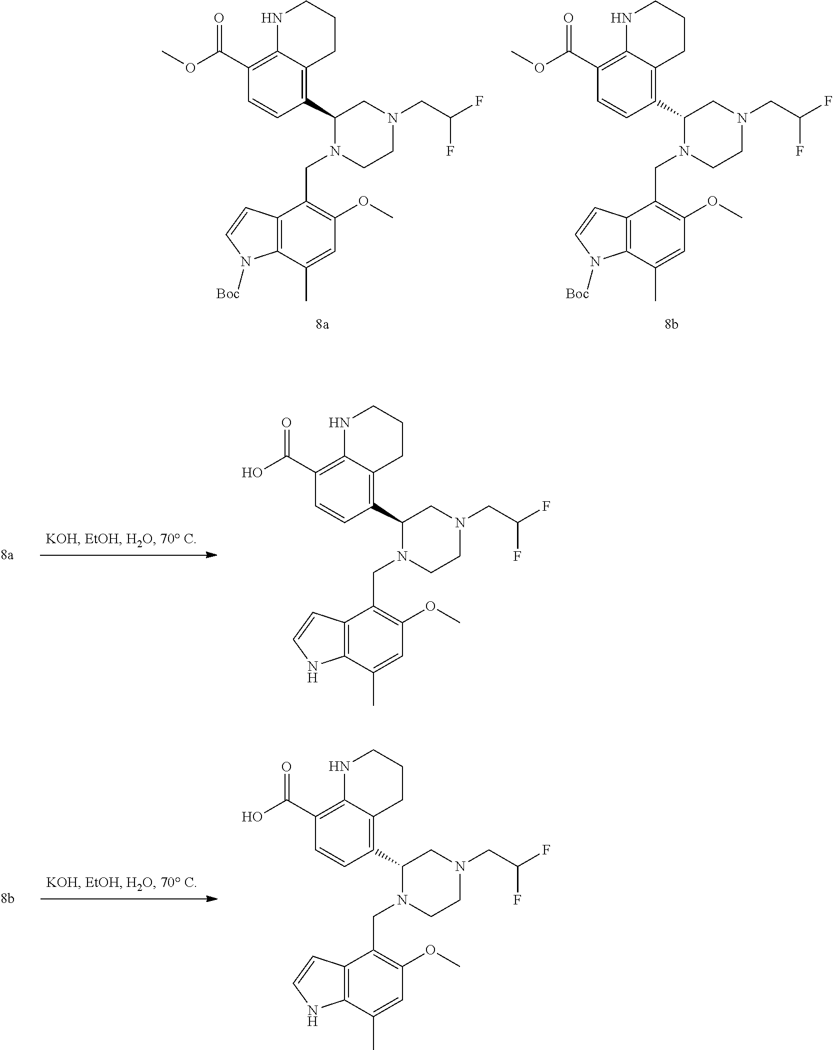

Step 1: Methyl 5-(pyrazin-2-yl) quinoline-8-carboxylate. A mixture of methyl 5-bromoquinoline-8-carboxylate (2 g, 7.51 mmol) and bis(pinacolato)diboron (2.01 g, 7.9 mmol), Pd(dppf)Cl₂ (0.55 g, 0.75 mmol), KOAc (1.84 g, 18.75 mmol) in dioxane (20 mL) was stirred for 1 h at 90° C. under nitrogen atmosphere. To the above mixture was added 2-chloropyrazine (1.04 g, 9.08 mmol), Pd(dppf)Cl₂ (0.55 g, 0.75 mmol), Sodium carbonate (1.6 g, 15.10 mmol) and water (2 mL). The mixture was stirred for 1 h at 90° C. under nitrogen atmosphere. The reaction was quenched with water and extracted with ethyl acetate. The solvent was removed under vacuum. The residue was purified by silica gel column chromatography, eluted with petroleum ether/ethyl acetate (1:1) to afford the title compound (1.53 g, 90% yield) as a yellow solid. LCMS (ESI): [M+H]⁺=266.1.

Step 2: Methyl 5-(piperazin-2-yl)-1,2,3,4-tetrahydroquinoline-8-carboxylate. A mixture of methyl 5-(pyrazin-2-yl) quinoline-8-carboxylate (1.53 g, 5.76 mmol) and Palladium (II) Acetate (0.65 g, 2.88 mmol) in acetic acid (20 mL) was stirred for overnight at room temperature under hydrogen. The solids were filtered out. The filtrate was concentrated under reduced pressure. The residue was purified by reverse phase flash to afford the title compound (500 mg, 80% yield) as a brown solid. LCMS (ESI): [M+H]⁺=276.2.

Step 3: Methyl 5-(4-(2,2-difluoroethyl)piperazin-2-yl)-1,2,3,4-tetrahydroquinoline-8-carboxylate. To a stirred solution of methyl 5-(piperazin-2-yl)-1,2,3,4-tetrahydroquinoline-8-carboxylate (423 mg, 1.54 mmol) and N,N-diisopropylethylamine (794 mg, 6.14 mmol) in ACN (20 mL) was added 2,2-difluoroethyl trifluoromethanesulfonate (263 mg, 1.23 mmol) dropwise in portions at 0° C. The resulting mixture was concentrated under vacuum. The residue was purified by reversed-phase flash chromatography to afford the title compound (260 mg, 90% yield) as a yellow oil. LCMS (ESI): [M+H]⁺=340.2.

Step 4: Methyl 5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate. To a mixture of Methyl 5-(4-(2,2-difluoroethyl)piperazin-2-yl)-1,2,3,4-tetrahydroquinoline-8-carboxylate (260 mg, 0.77 mmol), tert-butyl 4-formyl-5-methoxy-7-methylindole-1-carboxylate (274 mg, 0.92 mmol), acetic acid (138 mg, 2.30 mmol) and Sodium triacetoxyborohydride (488 mg, 2.30 mmol) in dichloroethane (7 mL) was stirred for 6 h at 70° C. The solvent was removed under reduced pressure. The residue was purified by silica gel column chromatography, eluted with petroleum ether/ethyl acetate (3:1) to afford the title compound (200 mg, 95% yield) as a yellow oil. LCMS (ESI): [M+H]⁺=613.3.

Step 5: Methyl(S)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 1, faster peak) & Methyl (R)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 2, slower peak).

Methyl 5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (240 mg) was applied for further separation by Chiral HPLC.

Methyl (S)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 1, faster peak) (100 mg, 41.7% yield). LCMS (ESI): [M+H]⁺=613.3. tR=1.57 min (CHIRALPAK IH-3, 4.6*50 mm 3 μm), Hex(0.1% DEA):Ethanol=98:2, 1.0 mL/min, 254 nm).

Methyl (R)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 2, slower peak) (100 mg, 41.7% yield). LCMS (ESI): [M+H]⁺=613.3. tR=1.90 min (CHIRALPAK IH-3, 4.6*50 mm 3 μm), Hex(0.1% DEA):Ethanol=98:2, 1.0 mL/min, 254 nm).

Step 6: (S)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid & (R)-5-(4-(2,2-difluoroethyl)-1-((5-methoxy-7-methyl-1H-indol-4-yl)methyl)piperazin-2-yl)-1,2,3,4-tetrahydroquinoline-8-carboxylic acid.

A mixture of Methyl (S)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 1, faster peak) (100 mg, 0.16 mmol) and Potassium hydroxide (92 mg, 1.64 mmol) in water (0.2 mL) and ethanol (1 mL) was stirred for 4 h at 70° C. The mixture was adjusted to pH=6 with sta. citric acid. The solvent was removed. The residue was purified by reversed-phase flash chromatography. This resulted in (S)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid (31.5 mg, 38.7% yield) as a white solid. LCMS (ESI): [M+H]⁺=499.2. ¹H NMR (300 MHz, DMSO-d₆) δ 10.83 (s, 1H), 7.69 (d, J=8.4 Hz, 1H), 7.26 (t, J=2.7 Hz, 1H), 7.01 (d, J=8.5 Hz, 1H), 6.67 (s, 1H), 6.50 (t, J=2.4 Hz, 1H), 6.13 (t, J=4.3 Hz, 1H), 3.72-3.67 (m, 3H), 3.54 (d, J=9.6 Hz, 1H), 3.33-3.30 (m, 3H), 3.03-2.89 (m, 1H), 2.87-2.70 (m, 1H), 2.59-2.46 (m, 6H), 2.43 (s, 3H), 2.34-2.07 (m, 3H), 2.07-1.73 (m, 2H).

A mixture of Methyl (R)-5-(1-((1-(tert-butoxycarbonyl)-5-methoxy-7-methyl-1H-indol-4-yl)methyl)-4-(2,2-difluoroethyl)piperazin-2-yl)-1,2,3,4-tetrahydroquinoline-8-carboxylate (isomer 2, slower peak) (100 mg, 0.16 mmol) and Potassium hydroxide (92 mg, 1.64 mmol) in water (0.2 mL) and ethanol (1 mL) was stirred for 4 h at 70° C. The mixture was adjusted to pH=6 with sta. citric acid. The solvent was removed. The residue was purified by reversed-phase flash chromatography. This resulted in (R)-5-(4-(2,2-Difluoroethyl)-1-((5-methoxy-7-methyl-1H-indol-4-yl)methyl)piperazin-2-yl)-1,2,3,4-tetrahydroquinoline-8-carboxylic acid (35.6 mg, 44.3% yield) as a white solid. LCMS (ESI): [M+H]⁺=499.2. ¹H NMR (300 MHz, DMSO-d₆) δ 10.83 (s, 1H), 7.69 (d, J=8.4 Hz, 1H), 7.26 (t, J=2.7 Hz, 1H), 7.01 (d, J=8.5 Hz, 1H), 6.67 (s, 1H), 6.50 (t, J=2.4 Hz, 1H), 6.13 (t, J=4.3 Hz, 1H), 3.72-3.67 (m, 3H), 3.54 (d, J=9.6 Hz, 1H), 3.33-3.30 (m, 3H), 3.03-2.89 (m, 1H), 2.87-2.70 (m, 1H), 2.59-2.46 (m, 6H), 2.43 (s, 3H), 2.34-2.07 (m, 3H), 2.07-1.73 (m, 2H).

### Example 25. (S)-4-(4-(2,2-Difluoroethyl)-1-((5-methoxy-7-methyl-1H-indol-4-yl)methyl)piperazin-2-yl)-2-(pyrrolidin-1-yl)benzoic acid & (R)-4-(4-(2,2-difluoroethyl)-1-((5-methoxy-7-methyl-1H-indol-4-yl)methyl)piperazin-2-yl)-2-(pyrrolidin-1-yl)benzoic acid

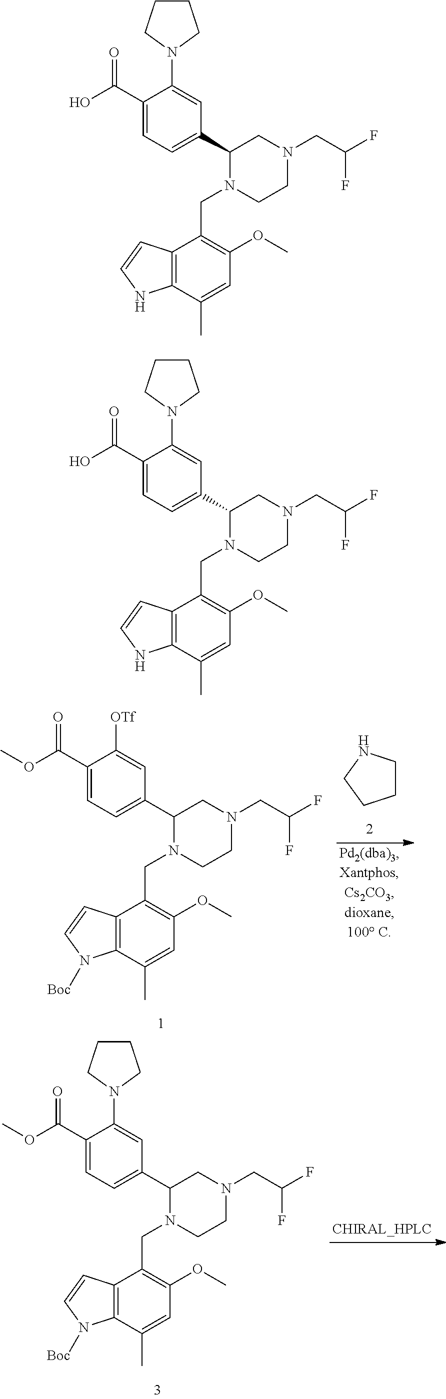

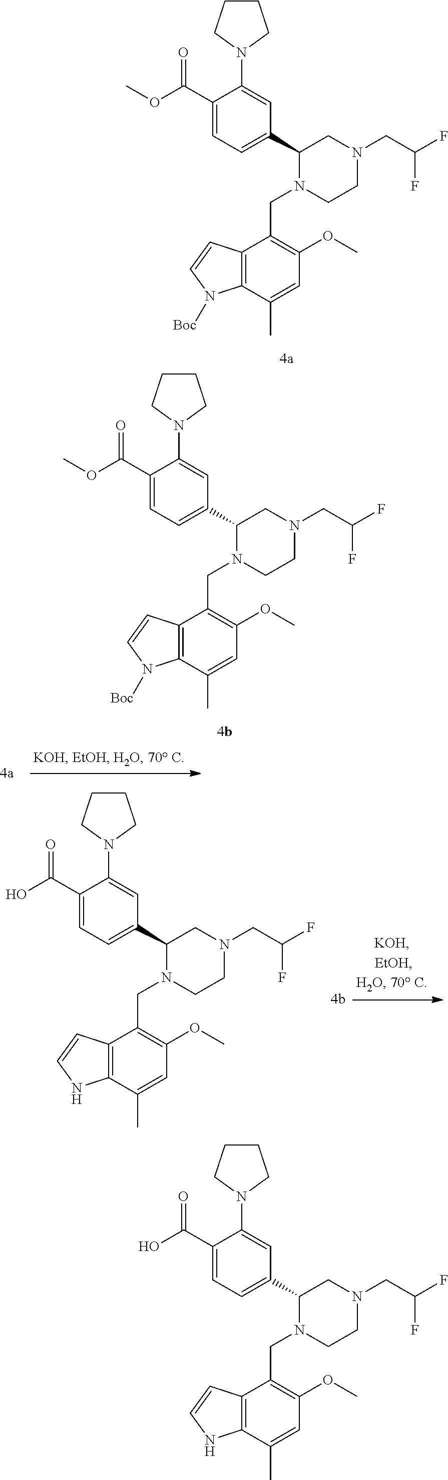 [SMILES: COC(=O)c1ccc([C@H]2CN(CC(F)F)CCN2Cc2c(OC)cc(C)c3c2ccn3C(=O)OC(C)(C)C)cc1N1CCCC1 ; COC(=O)c1ccc([C@@H]2CN(CC(F)F)CCN2Cc2c(OC)cc(C)c3c2ccn3C(=O)OC(C)(C)C)cc1N1CCCC1 ; COc1cc(C)c2[nH]ccc2c1CN1CCN(CC(F)F)C[C@@H]1c1ccc(C(=O)O)c(N2CCCC2)c1 ; COc1cc(C)c2[nH]ccc2c1CN1CCN(CC(F)F)C[C@H]1c1ccc(C(=O)O)c(N2CCCC2)c1]

Step 1: tert-Butyl 4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate. Under nitrogen, a solution of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (300 mg, 0.42 mmol), pyrrolidine (91 mg, 1.27 mmol), Tris(dibenzylideneacetone)dipalladium (78 mg, 0.08 mmol), XantPhos (74 mg, 0.12 mmol) and Cesium carbonate (415 mg, 1.27 mmol) in dioxane (5 mL) was stirred for 2 h at 100° C. The solvent was removed under reduced pressure. The residue was purified by silica gel column chromatography, eluted with ethyl acetate/petroleum ether (1:1) to afford the title compound (160 mg, 60.8% yield) as a yellow solid. LCMS (ESI): [M+H]⁺=627.3.

Step 2: tert-Butyl (S)-4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate (isomer 1, faster peak) & tert-butyl (R)-4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate (isomer 2, slower peak).

tert-Butyl 4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate (160 mg) was applied for further separation by Chiral HPLC.

tert-Butyl (S)-4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate (isomer 1, faster peak). LCMS (ESI): [M+H]⁺=627.3. tR=1.99 min (CHIRALPAK ID-3, 4.6*50 mm, 3 μm, Hex(0.1% DEA):IPA=90:10, 1.0 mL/min, 254 nm).

tert-Butyl (R)-4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate (isomer 1, slower peak). LCMS (ESI): [M+H]⁺=627.3. tR=2.71 min (CHIRALPAK ID-3, 4.6*50 mm, 3 um, Hex(0.1% DEA):IPA=90:10, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-(pyrrolidin-1-yl)benzoic acid & (R)-4-(4-(2,2-difluoroethyl)-1-((5-methoxy-7-methyl-1H-indol-4-yl)methyl)piperazin-2-yl)-2-(pyrrolidin-1-yl)benzoic acid.

A solution of tert-Butyl (S)-4-((4-(2,2-difluoroethyl)-2-(4-(methoxycarbonyl)-3-(pyrrolidin-1-yl)phenyl)piperazin-1-yl)methyl)-5-methoxy-7-methyl-1H-indole-1-carboxylate (50 mg, 0.08 mmol) in ethanol (3 mL) and water water (0.5 mL) was added Potassium hydroxide (100 mg, 1.78 mmol) and stirred for 2 h at 70° C. The mixture was acidified to pH=6 with sat. citric acid solution. The solvent was removed under vacuum. The residue was purified by reversed-phase flash chromatography to afford the title compound.

(S)-4-(4-(2,2-Difluoroethyl)-1-((5-methoxy-7-methyl-1H-indol-4-yl)methyl)piperazin-2-yl)-2-(pyrrolidin-1-yl)benzoic acid (16.1 mg, 38.8% yield) as a white solid. LCMS (ESI) [M+H]⁺=513.3, ¹H NMR (300 MHz, Methanol-d₄) δ 7.75 (d, J=7.8 Hz, 1H), 7.33-7.10 (m, 3H), 6.70 (s, 1H), 6.41 (d, J=3.1 Hz, 1H), 6.21-5.70 (m, 1H), 4.20-3.80 (m, 2H), 3.78-3.70 (m, 4H), 3.56-3.36 (m, 4H), 3.18-3.04 (m, 1H), 2.97 (t, J=10.6 Hz, 2H), 2.85-2.68 (m, 3H), 2.59 (d, J=11.6 Hz, 2H), 2.48-2.41 (m, 3H), 2.18-1.91 (m, 4H).

(R)-4-(4-(2,2-Difluoroethyl)-1-((5-methoxy-7-methyl-1H-indol-4-yl)methyl)piperazin-2-yl)-2-(pyrrolidin-1-yl)benzoic acid was prepared according to above.

(R)-4-(4-(2,2-Difluoroethyl)-1-((5-methoxy-7-methyl-1H-indol-4-yl)methyl)piperazin-2-yl)-2-(pyrrolidin-1-yl)benzoic acid (16.5 mg, 39.1% yield) as a white solid. LCMS (ESI) [M+H]⁺=513.3, ¹H NMR (300 MHz, Methanol-d₄) δ 7.75 (d, J=7.8 Hz, 1H), 7.33-7.10 (m, 3H), 6.70 (s, 1H), 6.41 (d, J=3.1 Hz, 1H), 6.21-5.70 (m, 1H), 4.20-3.80 (m, 2H), 3.78-3.70 (m, 4H), 3.56-3.36 (m, 4H), 3.18-3.04 (m, 1H), 2.97 (t, J=10.6 Hz, 2H), 2.85-2.68 (m, 3H), 2.59 (d, J=11.6 Hz, 2H), 2.48-2.41 (m, 3H), 2.18-1.91 (m, 4H).

### Example 26. 4-((3R,8aS)-2-((5-Methoxy-7-methyl-1H-indol-4-yl)methyl)octahydropyrrolo[1,2-a]pyrazin-3-yl)-2-(methylamino)benzoic acid

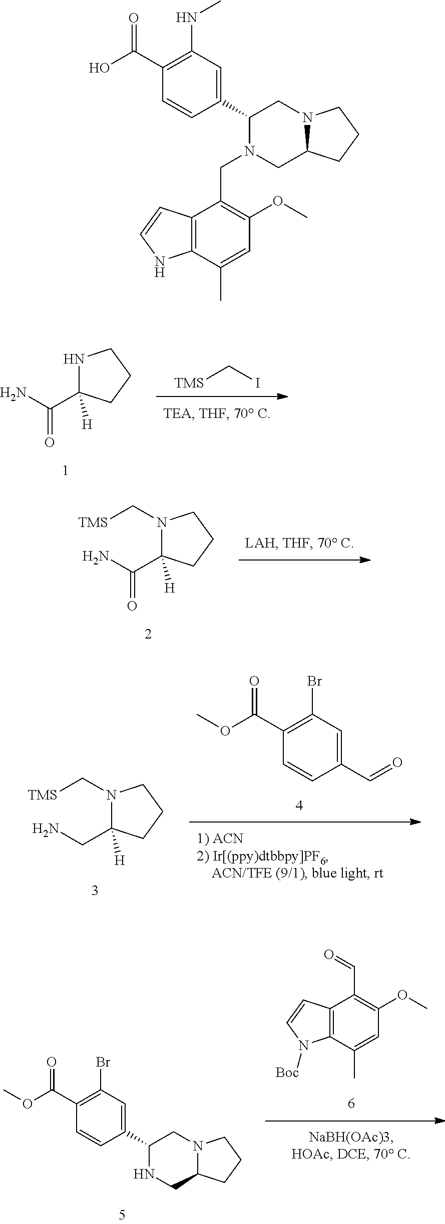

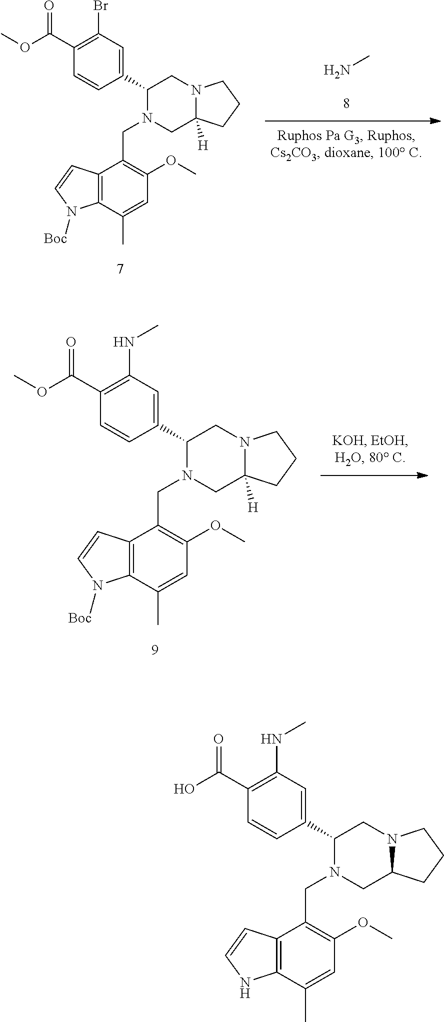

Step 1: (2S)-1-((Trimethylsilyl)methyl)pyrrolidine-2-carboxamide. A solution of prolinamide (10.00 g, 87.61 mmol) in THF (60 mL) was added (iodomethyl)trimethylsilane (37.52 g, 175.21 mmol) and TEA (26.60 g, 262.82 mmol) and stirred at 70° C. under nitrogen atmosphere for overnight. The reaction was cooled to room temperature and condensed under vacuo. The residue was purified by reverse phase chromatography (Acetonitrile 0-40%/0.1% Ammonium bicarbonate in water) to afford the title compound (15.70 g, 89.4% yield) as a dark yellow oil. LCMS (ESI): [M+H]⁺=201.1.

Step 2: 1-((2S)-1-((Trimethylsilyl)methyl)pyrrolidin-2-yl)methanamine. To a solution of (2S)-1-((trimethylsilyl)methyl)pyrrolidine-2-carboxamide (2.00 g, 9.98 mmol) in THF (40 mL) was added Lithium Aluminum Hydride (1.14 g, 29.95 mmol) at 0° C. The mixture was stirred for 15 min and the mixture was allowed to warm to 70° C. stirred for 5 hours. The reaction mixture was quenched by water, 15% NaOH(aq) and water. The solids were removed. The filtrate was concentrated under vacuum to afford the title compound (1.56 g, 83.8% yield). LCMS (ESI): [M+H]⁺=187.1.

Step 3: Methyl 4-((3R,8aS)-octahydropyrrolo(1,2-a)pyrazin-3-yl)-2-bromobenzoate. A solution of 1-((2S)-1-((trimethylsilyl)methyl)pyrrolidin-2-yl)methanamine (1.00 g, 5.36 mmol) and methyl 2-bromo-4-formylbenzoate (1.30 g, 5.37 mmol) in ACN (9 mL) was stirred for 18 h at room temperature. Then Difluoroethanol (1 mL) and (Ir(dtbbpy)(ppy)₂)(PF₆) (490 mg, 0.54 mmol) was added and the reaction continued to stirred at room temparature for 3 hours under blue LED. The solvent was removed and the residue was purified by reverse phase chromatography (Acetonitrile 0-30%/0.1% Ammonium bicarbonate in water) to afford the title compound (200 mg, 10.9% yield) as a yellow oil. LCMS (ESI): [M+H]⁺=339.1.

Step 4: tert-Butyl 4-(((3R,8aS)-3-(3-bromo-4-(methoxycarbonyl)phenyl)-hexahydro-1H-pyrrolo(1,2-a)pyrazin-2-yl)methyl)-5-methoxy-7-methylindole-1-carboxylate. A solution of methyl 4-((3R,8aS)-octahydropyrrolo(1,2-a)pyrazin-3-yl)-2-bromobenzoate (180 mg, 0.53 mmol) in dichloromethane (2 mL) was added tert-butyl 4-formyl-5-methoxy-7-methylindole-1-carboxylate (230 mg, 0.79 mmol), HOAc (32 mg, 0.53 mmol) and Sodium triacetoxyborohydride (225 mg, 1.06 mmol) at room temperature. The resulting mixture was stirred for 3 hours at 70° C. The solvent was concentrated under vacuum and the residue was purified by flash with Sodium triacetoxyborohydride (7/3) to afford the title compound (170 mg, 52.3% yield) as a yellow oil. LCMS (ESI): [M+H]⁺=612.2.

Step 5: tert-Butyl 4-(((3R,8aS)-3-(4-(methoxycarbonyl)-3-(methylamino)phenyl)-hexahydro-1H-pyrrolo(1,2-a)pyrazin-2-yl)methyl)-5-methoxy-7-methylindole-1-carboxylate. A solution of tert-butyl 4-(((3R,8aS)-3-(3-bromo-4-(methoxycarbonyl)phenyl)-hexahydro-1H-pyrrolo(1,2-a)pyrazin-2-yl)methyl)-5-methoxy-7-methylindole-1-carboxylate (130 mg, 0.21 mmol) in 1,4-dioxane (2 mL) was added 1 M methylamine in THF (1.06 mL, 1.06 mmol), RuPhos Pd G3 (18 mg, 0.02 mmol), RuPhos (20 mg, 0.04 mmol) and Cs₂CO₃ (207 mg, 0.64 mmol) and stirred for 18 hours at 100° C. under nitrogen atmosphere. The solvent was concentrated under vacuum and the residue was purified by silica gel column eluting with petroleum ether/ethyl acetate (0-95%) to afford the title compound (30 mg, 25.1% yield) as a yellow solid. LCMS (ESI): [M+H]⁺=563.3.

Step 6: 4-((3R,8aS)-2-((5-Methoxy-7-methyl-1H-indol-4-yl)methyl)octahydropyrrolo[1,2-a]pyrazin-3-yl)-2-(methylamino)benzoic acid. A solution of tert-butyl 4-(((3R,8aS)-3-(4-(methoxycarbonyl)-3-(methylamino)phenyl)-hexahydro-1H-pyrrolo(1,2-a)pyrazin-2-yl)methyl)-5-methoxy-7-methylindole-1-carboxylate (30 mg, 0.05 mmol) in Ethanol (1 mL) was added Potassium hydroxide (60 mg, 1.06 mmol) in water (0.2 mL) and stirred for 1.5 hours at 80° C. The mixture was allowed to cool down to room temperature and was neutralized to pH=6 with sat. Citric acid. The resulting residue was purified by reverse phase chromatography (Acetonitrile 0-30%/0.1% Ammonium bicarbonate in water) to afford the title compound (13.2 mg, 53.9% yield) as a white solid. LCMS (ESI): [M+H]⁺=449.2. ¹H NMR (300 MHz, DMSO-d₆) δ 10.84 (s, 1H), 7.80 (d, J=8.1 Hz, 1H), 7.26 (t, J=2.8 Hz, 1H), 6.96-6.75 (m, 2H), 6.67 (s, 1H), 6.62-6.53 (m, 1H), 3.71 (s, 4H), 3.18 (s, 2H), 2.93 (d, J=14.5 Hz, 5H), 2.77 (d, J=9.9 Hz, 1H), 2.42 (s, 3H), 2.19 (t, J=10.3 Hz, 1H), 2.08-1.79 (m, 3H), 1.75-1.52 (m, 3H), 1.21 (d, J=14.0 Hz, 1H).

### Example 27. (S)-2-((3,3-Difluorocyclobutyl)amino)-4-(4-(2,2-difluoroethyl)-1-((5-methoxy-7-methyl-1H-indol-4-yl)methyl)piperazin-2-yl)benzoic acid & (R)-2-((3,3-difluorocyclobutyl)amino)-4-(4-(2,2-difluoroethyl)-1-((5-methoxy-7-methyl-1H-indol-4-yl)methyl)piperazin-2-yl)benzoic acid

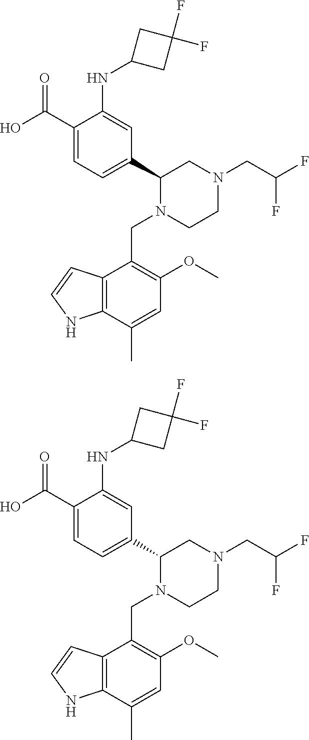 [SMILES: COc1cc(C)c2[nH]ccc2c1CN1CCN(CC(F)F)C[C@@H]1c1ccc(C(=O)O)c(NC2CC(F)(F)C2)c1 ; COc1cc(C)c2[nH]ccc2c1CN1CCN(CC(F)F)C[C@H]1c1ccc(C(=O)O)c(NC2CC(F)(F)C2)c1]

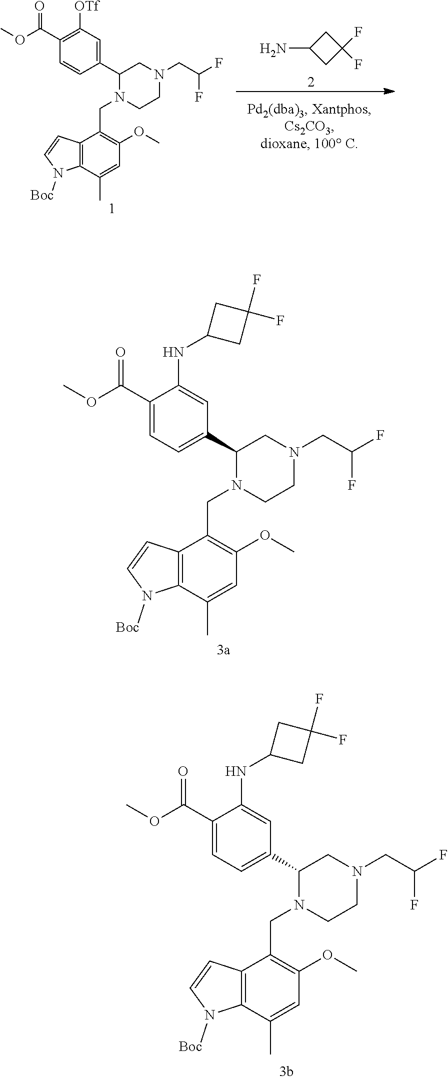 [SMILES: COC(=O)c1ccc(C2CN(CC(F)F)CCN2Cc2c(OC)cc(C)c3c2ccn3C(=O)OC(C)(C)C)cc1OS(=O)(=O)C(F)(F)F ; NC1CC(F)(F)C1 ; COC(=O)c1ccc([C@H]2CN(CC(F)F)CCN2Cc2c(OC)cc(C)c3c2ccn3C(=O)OC(C)(C)C)cc1NC1CC(F)(F)C1 ; COC(=O)c1ccc([C@@H]2CN(CC(F)F)CCN2Cc2c(OC)cc(C)c3c2ccn3C(=O)OC(C)(C)C)cc1NC1CC(F)(F)C1]

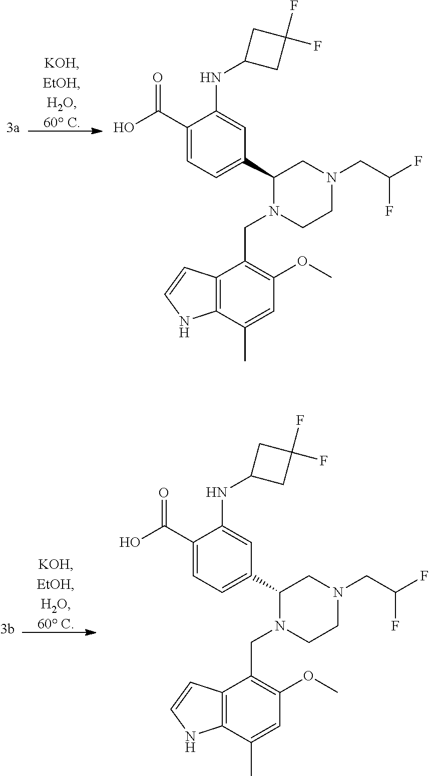 [SMILES: COc1cc(C)c2[nH]ccc2c1CN1CCN(CC(F)F)C[C@@H]1c1ccc(C(=O)O)c(NC2CC(F)(F)C2)c1 ; COc1cc(C)c2[nH]ccc2c1CN1CCN(CC(F)F)C[C@H]1c1ccc(C(=O)O)c(NC2CC(F)(F)C2)c1]

Step 1: tert-Butyl (S)-4-((2-(3-((3,3-difluorocyclobutyl)amino)-4-(methoxycarbonyl)phenyl)-4-(2,2-difluoroethyl)piperazin-1-yl)methyl)-5-methoxy-7-methyl-1H-indole-1-carboxylate & tert-butyl (R)-4-((2-(3-((3,3-difluorocyclobutyl)amino)-4-(methoxycarbonyl)phenyl)-4-(2,2-difluoroethyl)piperazin-1-yl)methyl)-5-methoxy-7-methyl-1H-indole-1-carboxylate.

A mixture of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (400 mg, 0.56 mmol), Cs₂CO₃ (560 mg, 1.72 mmol), Pd₂(dba)₃ (60 mg, 0.07 mmol), XantPhos (70 mg, 0.12 mmol) and 3,3-difluorocyclobutan-1-amine (200 mg, 1.86 mmol) in dioxane (4.5 mL) was stirred for 2 h at 100° C. under nitrogen atmosphere. The solids were filtered out, the filtrate was concentrated under reduced pressure. The residue was purified by silica gel column chromatography, eluted with ethyl acetate/petroleum ether (0-25%) to afford the product as racemate. Then the racemate was applied for further separation by Chiral HPLC to afford the title compounds.

tert-Butyl (S)-4-((2-(3-((3,3-difluorocyclobutyl)amino)-4-(methoxycarbonyl)phenyl)-4-(2,2-difluoroethyl)piperazin-1-yl)methyl)-5-methoxy-7-methyl-1H-indole-1-carboxylate (83 mg, 22.0% yield, faster peak, isomer 1), LCMS (ESI): [M+H]⁺=663.3, tR=1.80 min (CHIRALPAK AD-3 (0.46*5 cm,3 um), Hex(0.1% DEA):IPA=98:2, 1.0 mL/min, 254 nm).

tert-butyl (R)-4-((2-(3-((3,3-difluorocyclobutyl)amino)-4-(methoxycarbonyl)phenyl)-4-(2,2-difluoroethyl)piperazin-1-yl)methyl)-5-methoxy-7-methyl-1H-indole-1-carboxylate (91 mg, 24.20% yield, slower peak, isomer 2), LCMS (ESI): [M+H]⁺=663.3, tR=2.81 min (CHIRALPAK AD-3 (0.46*5 cm,3 um), Hex(0.1% DEA):IPA-98:2, 1.0 mL/min, 254 nm).

Step 2: (S)-2-((3,3-Difluorocyclobutyl)amino)-4-(4-(2,2-difluoroethyl)-1-((5-methoxy-7-methyl-1H-indol-4-yl)methyl)piperazin-2-yl)benzoic acid & (R)-2-((3,3-difluorocyclobutyl)amino)-4-(4-(2,2-difluoroethyl)-1-((5-methoxy-7-methyl-1H-indol-4-yl)methyl)piperazin-2-yl)benzoic acid.

A mixture of tert-Butyl (S)-4-((2-(3-((3,3-difluorocyclobutyl)amino)-4-(methoxycarbonyl)phenyl)-4-(2,2-difluoroethyl)piperazin-1-yl)methyl)-5-methoxy-7-methyl-1H-indole-1-carboxylate (83 mg, 0.12 mmol, faster peak, isomer 1) and Potassium hydroxide (166 mg, 2.95 mmol) in water (1 mL) and Ethanol (2 mL) was stirred for 6 h at 60° C. The mixture was neutralized to pH 7 with sat. citric acid. The crude product was purified by reverse phase flash (ACN/10 mM NH₄HCO₃ in water) to afford (S)-2-((3,3-Difluorocyclobutyl)amino)-4-(4-(2,2-difluoroethyl)-1-((5-methoxy-7-methyl-1H-indol-4-yl)methyl)piperazin-2-yl)benzoic acid (39 mg, 57.6% yield) as a white solid. LCMS (ESI): [M+H]⁺=549.2. ¹H NMR (300 MHz, DMSO-d₆) δ 12.68 (s, 1H), 10.82 (s, 1H), 8.03 (s, 1H), 7.83 (d, J=8.2 Hz, 1H), 7.23 (t, J=2.8 Hz, 1H), 6.90 (m, 1H), 6.81-6.49 (m, 3H), 6.31-5.89 (m, 1H), 3.97 (s, 1H), 3.69 (m, 4H), 3.16 (m, 4H), 2.84-2.54 (m, 6H), 2.41 (s, 4H), 2.27 (t, J=10.5 Hz, 1H), 2.22-2.10 (m, 2H).

A mixture of tert-Butyl (R)-4-((2-(3-((3,3-difluorocyclobutyl)amino)-4-(methoxycarbonyl)phenyl)-4-(2,2-difluoroethyl)piperazin-1-yl)methyl)-5-methoxy-7-methyl-1H-indole-1-carboxylate (81 mg, 0.12 mmol, slower peak, isomer 2) and Potassium hydroxide (162 mg, 2.88 mmol) in water (1.5 mL) and Ethanol (3 mL) was stirred for 6 h at 60° C. The mixture was neutralized to pH 7 with sat. citric acid. The crude product was purified by reverse phase flash (ACN/10 mM NH₄HCO₃ in water) to afford (R)-2-((3,3-Difluorocyclobutyl)amino)-4-(4-(2,2-difluoroethyl)-1-((5-methoxy-7-methyl-1H-indol-4-yl)methyl)piperazin-2-yl)benzoic acid (43 mg, 64.4% yield) as a white solid. LCMS (ESI): [M+H]⁺=549.3. ¹H NMR (300 MHz, DMSO-d₆) δ 12.67 (s, 1H), 10.82 (s, 1H), 8.06 (s, 1H), 7.83 (d, J=8.1 Hz, 1H), 7.23 (t, J=2.8 Hz, 1H), 6.89 (m, 1H), 6.79 (s, 1H), 6.65 (s, 1H), 6.52 (s, 1H), 6.10 (m, 1H), 3.97 (s, 1H), 3.69 (s, 3H), 3.60 (d, J=12.2 Hz, 1H), 3.28-3.23 (m, 1H), 3.14 (m, 3H), 2.85-2.54 (m, 6H), 2.41 (s, 4H), 2.27 (t, J=10.5 Hz, 1H), 2.17 (m, 2H).

### Example 28. (S)-4-(4-(2,2-Difluoroethyl)-1-((5-methoxy-7-methyl-1H-indol-4-yl)methyl)piperazin-2-yl)-2-((1-methyl-1H-pyrazol-4-yl)amino)benzoic acid & (R)-4-(4-(2,2-difluoroethyl)-1-((5-methoxy-7-methyl-1H-indol-4-yl)methyl)piperazin-2-yl)-2-((1-methyl-1H-pyrazol-4-yl)amino)benzoic acid

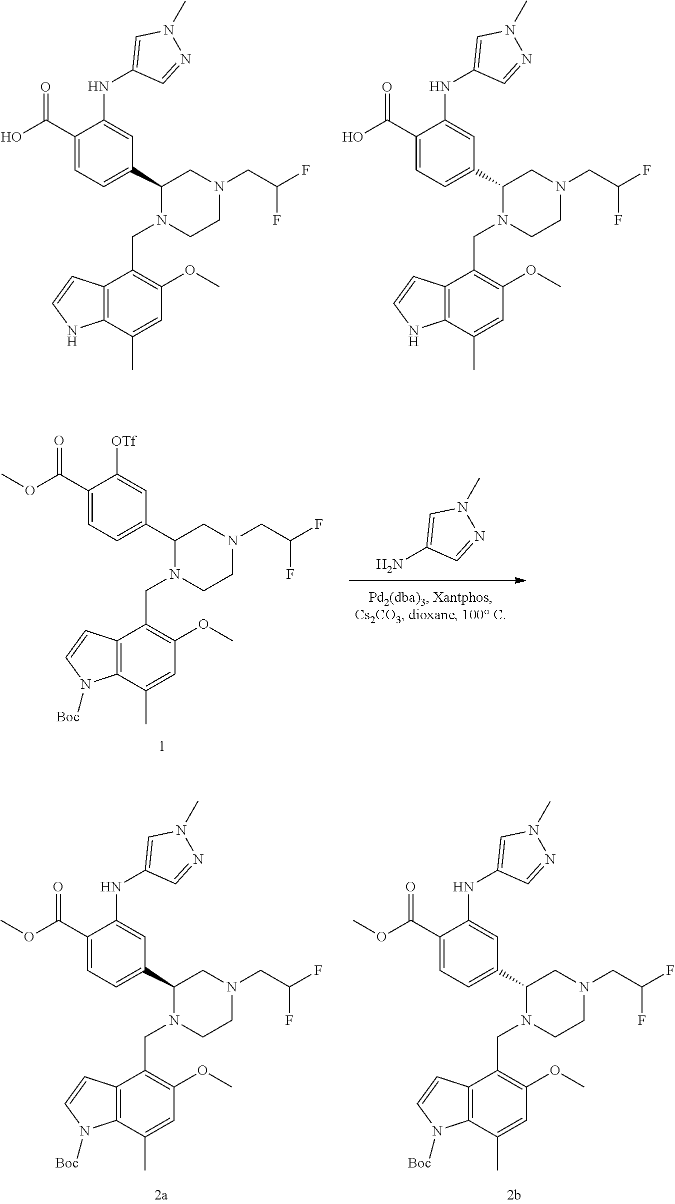

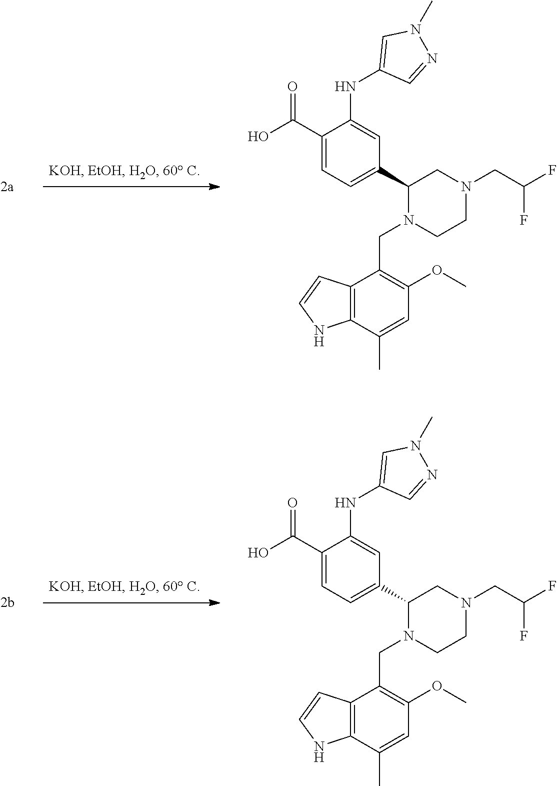 [SMILES: COc1cc(C)c2[nH]ccc2c1CN1CCN(CC(F)F)C[C@@H]1c1ccc(C(=O)O)c(Nc2cnn(C)c2)c1 ; COc1cc(C)c2[nH]ccc2c1CN1CCN(CC(F)F)C[C@H]1c1ccc(C(=O)O)c(Nc2cnn(C)c2)c1]

Step 1: tert-Butyl (S)-4-((4-(2,2-difluoroethyl)-2-(4-(methoxycarbonyl)-3-((1-methyl-1H-pyrazol-4-yl)amino)phenyl)piperazin-1-yl)methyl)-5-methoxy-7-methyl-1H-indole-1-carboxylate & tert-butyl (R)-4-((4-(2,2-difluoroethyl)-2-(4-(methoxycarbonyl)-3-((1-methyl-1H-pyrazol-4-yl)amino)phenyl)piperazin-1-yl)methyl)-5-methoxy-7-methyl-1H-indole-1-carboxylate.

A mixture of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (600 mg, 0.85 mmol), Cs₂CO₃ (831 mg, 2.55 mmol), Pd₂(dba)₃ (78 mg, 0.08 mmol), XantPhos (98 mg, 0.16 mmol) and 1-methylpyrazol-4-amine (248 mg, 2.55 mmol) in 1,4-dioxane (6 mL) was stirred for 2 h at 100° C. under nitrogen atmosphere. The solids were filtered out, the filtrate was concentrated under reduced pressure. The residue was purified by silica gel column chromatography, eluted with ethyl acetate/petroleum ether (0-70%) and further separated by Chiral-HPLC.

tert-Butyl (S)-4-((4-(2,2-difluoroethyl)-2-(4-(methoxycarbonyl)-3-((1-methyl-1H-pyrazol-4-yl)amino)phenyl)piperazin-1-yl)methyl)-5-methoxy-7-methyl-1H-indole-1-carboxylate (121 mg, 21.8% yield, faster peak, isomer 1), LCMS (ESI): [M+H]⁺=653.2, tR=1.20 min (CHIRALPAK IA-3 4.6*50 mm 3 um, Hex(0.1% DEA):IPA=70:30, 1.0 mL/min, 254 nm)

tert-butyl (R)-4-((4-(2,2-difluoroethyl)-2-(4-(methoxycarbonyl)-3-((1-methyl-1H-pyrazol-4-yl)amino)phenyl)piperazin-1-yl)methyl)-5-methoxy-7-methyl-1H-indole-1-carboxylate (114 mg, 45.6% yield, slower peak, isomer 2), LCMS (ESI): [M+H]⁺=653.2, tR=1.52 min (CHIRALPAK IA-3 4.6*50 mm 3 um, Hex(0.1% DEA):IPA=70:30, 1.0 mL/min, 254 nm).

Step 2: (S)-4-(4-(2,2-Difluoroethyl)-1-((5-methoxy-7-methyl-1H-indol-4-yl)methyl)piperazin-2-yl)-2-((1-methyl-1H-pyrazol-4-yl)amino)benzoic acid & (R)-4-(4-(2,2-difluoroethyl)-1-((5-methoxy-7-methyl-1H-indol-4-yl)methyl)piperazin-2-yl)-2-((1-methyl-1H-pyrazol-4-yl)amino)benzoic acid.

A mixture of tert-butyl (S)-4-((4-(2,2-difluoroethyl)-2-(4-(methoxycarbonyl)-3-((1-methyl-1H-pyrazol-4-yl)amino)phenyl)piperazin-1-yl)methyl)-5-methoxy-7-methyl-1H-indole-1-carboxylate (111 mg, 0.17 mmol) and Potassium hydroxide (222 mg, 3.95 mmol) in water (1.5 mL) and Ethanol (3 mL) was stirred for 4 h at 60° C. The mixture was neutralized to pH=6 with sat. citric acid. The crude product was purified by reverse phase flash with ACN/10 mM NH₄HCO₃ in water to afford (S)-4-(4-(2,2-Difluoroethyl)-1-((5-methoxy-7-methyl-1H-indol-4-yl)methyl)piperazin-2-yl)-2-((1-methyl-1H-pyrazol-4-yl)amino)benzoic acid (55 mg, 59.8% yield) as a light yellow solid. LCMS (ESI): [M+H]⁺=539.2. ¹H NMR (300 MHz, DMSO-d₆) δ 12.96-12.74 (m, 1H), 10.80 (s, 1H), 9.27 (s, 1H), 7.89-7.78 (m, 2H), 7.47 (d, J=0.8 Hz, 1H), 7.20 (m, 1H), 7.07 (s, 1H), 6.89 (s, 1H), 6.63 (s, 1H), 6.34 (s, 1H), 6.29-5.87 (m, 1H), 3.84 (s, 3H), 3.65 (s, 3H), 3.56 (d, J=11.9 Hz, 1H), 3.21 (m, 2H), 2.83-2.57 (m, 5H), 2.40 (s, 3H), 2.25-2.06 (m, 3H).

A mixture of tert-butyl (R)-4-((4-(2,2-difluoroethyl)-2-(4-(methoxycarbonyl)-3-((1-methyl-1H-pyrazol-4-yl)amino)phenyl)piperazin-1-yl)methyl)-5-methoxy-7-methyl-1H-indole-1-carboxylate (114 mg, 0.17 mmol) and Potassium hydroxide (228 mg, 4.06 mmol) in water (1.5 mL) and Ethanol (3 mL) was stirred for 4 h at 60° C. The mixture was neutralized to pH=7 with sat. citric acid. The crude product was purified by reverse phase flash with the ACN/10 mM NH₄HCO₃ to afford (R)-4-(4-(2,2-difluoroethyl)-1-((5-methoxy-7-methyl-1H-indol-4-yl)methyl)piperazin-2-yl)-2-((1-methyl-1H-pyrazol-4-yl)amino)benzoic acid (52 mg, 56.0% yield) as a light yellow solid. LCMS (ESI): [M+H]⁺=539.2. ¹H NMR (300 MHz, DMSO-d₆) δ 12.99-12.72 (m, 1H), 10.80 (s, 1H), 9.32 (s, 1H), 7.92-7.76 (m, 2H), 7.46 (s, 1H), 7.20 (t, J=2.7 Hz, 1H), 7.07 (s, 1H), 6.88 (s, 1H), 6.63 (s, 1H), 6.34 (s, 1H), 6.31-5.87 (m, 1H), 3.83 (s, 3H), 3.65 (s, 3H), 3.56 (d, J=11.9 Hz, 1H), 3.21 (t, J=10.0 Hz, 2H), 2.81-2.52 (m, 5H), 2.40 (s, 3H), 2.24-2.07 (m, 3H).

### Example 29. (S)-2-((Cyclopropylmethyl)amino)-4-(1-((5-methoxy-7-methyl-1H-indol-4-yl)methyl)-4-(3,3,3-trifluoropropyl)piperazin-2-yl)benzoic acid & (S)-2-((cyclopropylmethyl)amino)-4-(1-((5-methoxy-7-methyl-1H-indol-4-yl)methyl)-4-(3,3,3-trifluoropropyl)piperazin-2-yl)benzoic acid

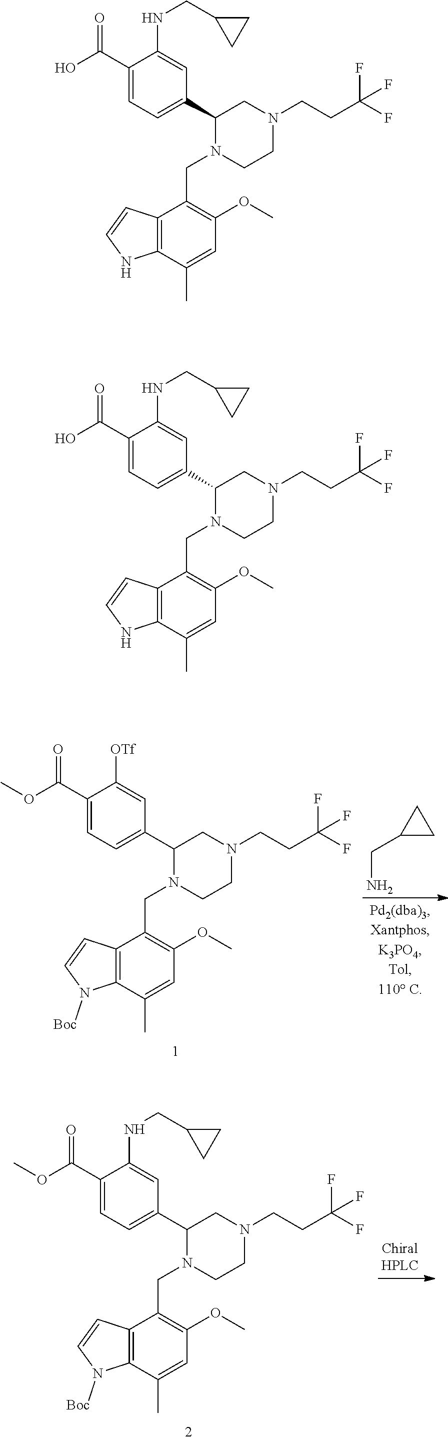

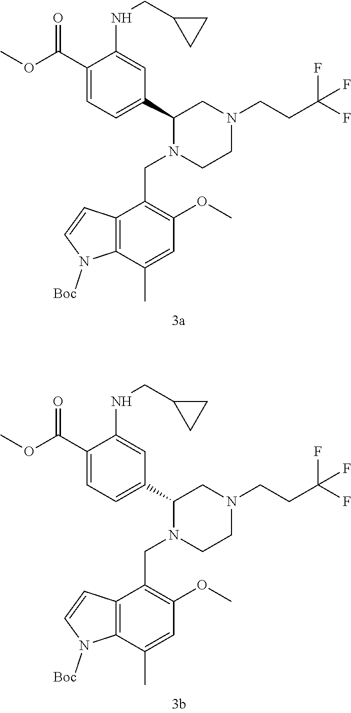 [SMILES: COC(=O)c1ccc([C@H]2CN(CCC(F)(F)F)CCN2Cc2c(OC)cc(C)c3c2ccn3C(=O)OC(C)(C)C)cc1NCC1CC1 ; COC(=O)c1ccc([C@@H]2CN(CCC(F)(F)F)CCN2Cc2c(OC)cc(C)c3c2ccn3C(=O)OC(C)(C)C)cc1NCC1CC1]

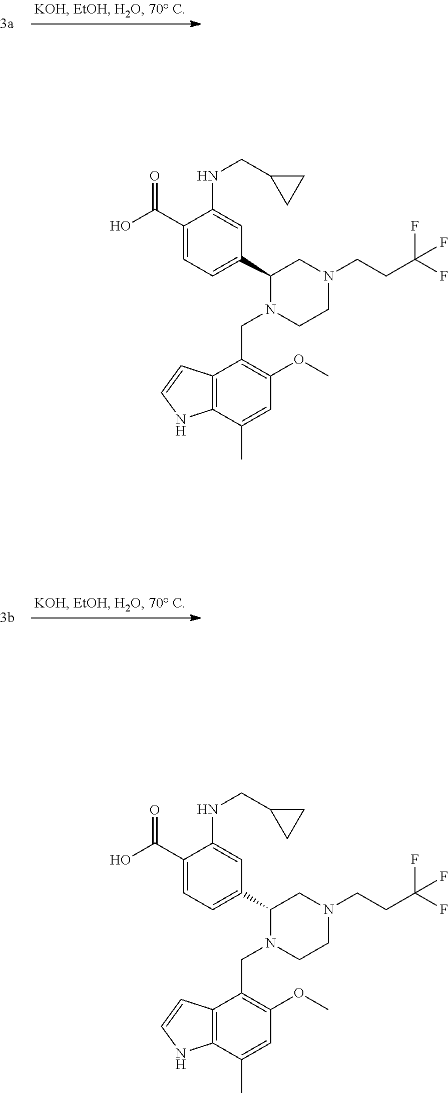 [SMILES: COc1cc(C)c2[nH]ccc2c1CN1CCN(CCC(F)(F)F)C[C@@H]1c1ccc(C(=O)O)c(NCC2CC2)c1 ; COc1cc(C)c2[nH]ccc2c1CN1CCN(CCC(F)(F)F)C[C@H]1c1ccc(C(=O)O)c(NCC2CC2)c1]

Step 1: tert-Butyl 4-((2-(3-((cyclopropylmethyl)amino)-4-(methoxycarbonyl)phenyl)-4-(3,3,3-trifluoropropyl)piperazin-1-yl)methyl)-5-methoxy-7-methyl-1H-indole-1-carboxylate. To a stirred solution of tert-butyl 5-methoxy-4-((2-(4-(methoxycarbonyl)-3-(trifluoromethanesulfonyloxy)phenyl)-4-(3,3,3-trifluoropropyl)piperazin-1-yl)methyl)-7-methylindole-1-carboxylate (300 mg, 0.41 mmol) in Toluene (5 mL) were added aminomethylcyclopropane (146 mg, 2.06 mmol), Potassium phosphate tribasic (258 mg, 1.22 mmol), Pd₂(dba)₃ (37 mg, 0.04 mmol), Xantphos (47 mg, 0.08 mmol) at room temperature. The resulting mixture was stirred for 4 h at 110° C. under nitrogen atmosphere. The solvent was removed under reduced pressure. The residue was purified by silica gel column chromatography, eluted with petroleum ether/ethyl acetate (2:1) to afford the title compound (210 mg, 78.4% yield) as a yellow solid. LCMS (ESI): [M+H]⁺=659.3.

Step 2: tert-Butyl 4-(((2S)-2-(3-((cyclopropylmethyl)amino)-4-propanoylphenyl)-5-fluoro-4-(3,3,3-trifluoropropyl)piperazin-1-yl)methyl)-5-methoxy-7-methylindole-1-carboxylate (isomer 1, faster peak) & tert-Butyl 4-(((2R)-2-(3-((cyclopropylmethyl)amino)-4-propanoylphenyl)-5-fluoro-4-(3,3,3-trifluoropropyl)piperazin-1-yl)methyl)-5-methoxy-7-methylindole-1-carboxylate (isomer 2, slower peak).

tert-Butyl 4-((2-(3-((cyclopropylmethyl)amino)-4-(methoxycarbonyl)phenyl)-4-(3,3,3-trifluoropropyl)piperazin-1-yl)methyl)-5-methoxy-7-methylindole-1-carboxylate (210 mg) was applied for further separation by Chiral HPLC.

tert-butyl 4-(((2S)-2-(3-((cyclopropylmethyl)amino)-4-propanoylphenyl)-5-fluoro-4-(3,3,3-trifluoropropyl)piperazin-1-yl)methyl)-5-methoxy-7-methylindole-1-carboxylate (isomer 1, faster peak) (100 mg, 47.6% yield). LCMS (ESI): [M+H]⁺=659.3. tR=1.78 min (CHIRALPAK AD-3 4.6*50 mm 3 μm, Hex(0.1% DEA):IPA=98:2, 1.0 mL/min, 254 nm).

tert-butyl 4-(((2R)-2-(3-((cyclopropylmethyl)amino)-4-propanoylphenyl)-5-fluoro-4-(3,3,3-trifluoropropyl)piperazin-1-yl)methyl)-5-methoxy-7-methylindole-1-carboxylate (isomer 2, slower peak) (100 mg, 47.6% yield). LCMS (ESI): [M+H]⁺=659.3. t_{R}=2.69 min (CHIRALPAK AD-3 4.6*50 mm 3 μm, Hex(0.1% DEA):IPA=98:2, 1.0 mL/min, 254 nm).

Step 3: (S)-2-((Cyclopropylmethyl)amino)-4-(1-((5-methoxy-7-methyl-1H-indol-4-yl)methyl)-4-(3,3,3-trifluoropropyl)piperazin-2-yl)benzoic acid & (S)-2-((cyclopropylmethyl)amino)-4-(1-((5-methoxy-7-methyl-1H-indol-4-yl)methyl)-4-(3,3,3-trifluoropropyl)piperazin-2-yl)benzoic acid.

To a stirred mixture of tert-butyl 4-(((2S)-2-(3-((cyclopropylmethyl)amino)-4-propanoylphenyl)-5-fluoro-4-(3,3,3-trifluoropropyl)piperazin-1-yl)methyl)-5-methoxy-7-methylindole-1-carboxylate (100 mg, 0.15 mmol) in Ethanol (2 mL) was added water (0.5 mL) and Potassium hydroxide (84 mg, 1.50 mmol). The reaction was stirred at 70° C. for 2 h. The mixture was allowed to cool down to room temperature and was neutralized to pH=6 with sat. citric acid. The solvent was removed. The residue was purified by reversed-phase flash chromatography with the following conditions: column, C18 silica gel; mobile phase, acetonitrile in Water (10 mmol/L NH₄HCO₃), 5% to 50% gradient in 20 min; detector, UV 254 nm. This resulted in (S)-2-((cyclopropylmethyl)amino)-4-(1-((5-methoxy-7-methyl-1H-indol-4-yl)methyl)-4-(3,3,3-trifluoropropyl)piperazin-2-yl)benzoic acid. (33.1 mg, 40.9% yield) as a white solid. LCMS (ESI): [M+H]⁺=545.3. ¹H NMR (300 MHz, DMSO-d₆) δ 10.83 (s, 1H), 7.79 (d, J=8.1 Hz, 1H), 7.22 (t, J=2.8 Hz, 1H), 6.94-6.82 (m, 1H), 6.75 (s, 1H), 6.64 (s, 1H), 6.52-6.47 (m, 1H), 3.68 (s, 3H), 3.62 (d, J=12.0 Hz, 1H), 3.31-3.18 (m, 2H), 3.07 (d, J=6.9 Hz, 2H), 2.83-2.66 (m, 2H), 2.60 (d, J=11.3 Hz, 1H), 2.47-2.29 (m, 7H), 2.22-1.87 (m, 3H), 1.19-1.06 (m, 1H), 0.59-0.47 (m, 2H), 0.30-0.16 (m, 2H).

To a stirred mixture of tert-butyl 4-(((2R)-2-(3-((cyclopropylmethyl)amino)-4-propanoylphenyl)-5-fluoro-4-(3,3,3-trifluoropropyl)piperazin-1-yl)methyl)-5-methoxy-7-methylindole-1-carboxylate (100 mg, 0.15 mmol) in Ethanol (2 mL) was added water (0.5 mL) and Potassium hydroxide (84 mg, 1.50 mmol). The reaction was stirred at 70° C. for 2 h. The mixture was allowed to cool down to room temperature and was neutralized to pH=6 with sat. citric acid. The solvent was removed. The residue was purified by reversed-phase flash chromatography with the following conditions: column, C18 silica gel; mobile phase, acetonitrile in Water (10 mmol/L NH₄HCO₃), 5% to 50% gradient in 20 min; detector, UV 254 nm. This resulted in (S)-2-((cyclopropylmethyl)amino)-4-(1-((5-methoxy-7-methyl-1H-indol-4-yl)methyl)-4-(3,3,3-trifluoropropyl)piperazin-2-yl)benzoic acid. (26.6 mg, 32.9% yield) as a white solid. LCMS (ESI): [M+H]⁺=545.3. ¹H NMR (300 MHz, DMSO-d₆) δ 10.83 (s, 1H), 7.79 (d, J=8.1 Hz, 1H), 7.22 (t, J=2.8 Hz, 1H), 6.94-6.82 (m, 1H), 6.75 (s, 1H), 6.64 (s, 1H), 6.52-6.47 (m, 1H), 3.68 (s, 3H), 3.62 (d, J=12.0 Hz, 1H), 3.31-3.18 (m, 2H), 3.07 (d, J=6.9 Hz, 2H), 2.83-2.66 (m, 2H), 2.60 (d, J=11.3 Hz, 1H), 2.47-2.29 (m, 7H), 2.22-1.87 (m, 3H), 1.19-1.06 (m, 1H), 0.59-0.47 (m, 2H), 0.30-0.16 (m, 2H).

### Example 30. (S)-2-Cyano-4-(1-((5-methoxy-7-methyl-1H-indol-4-yl)methyl)-4-(3,3,3-trifluoropropyl)piperazin-2-yl)benzoic acid & (R)-2-cyano-4-(1-((5-methoxy-7-methyl-1H-indol-4-yl)methyl)-4-(3,3,3-trifluoropropyl)piperazin-2-yl)benzoic acid

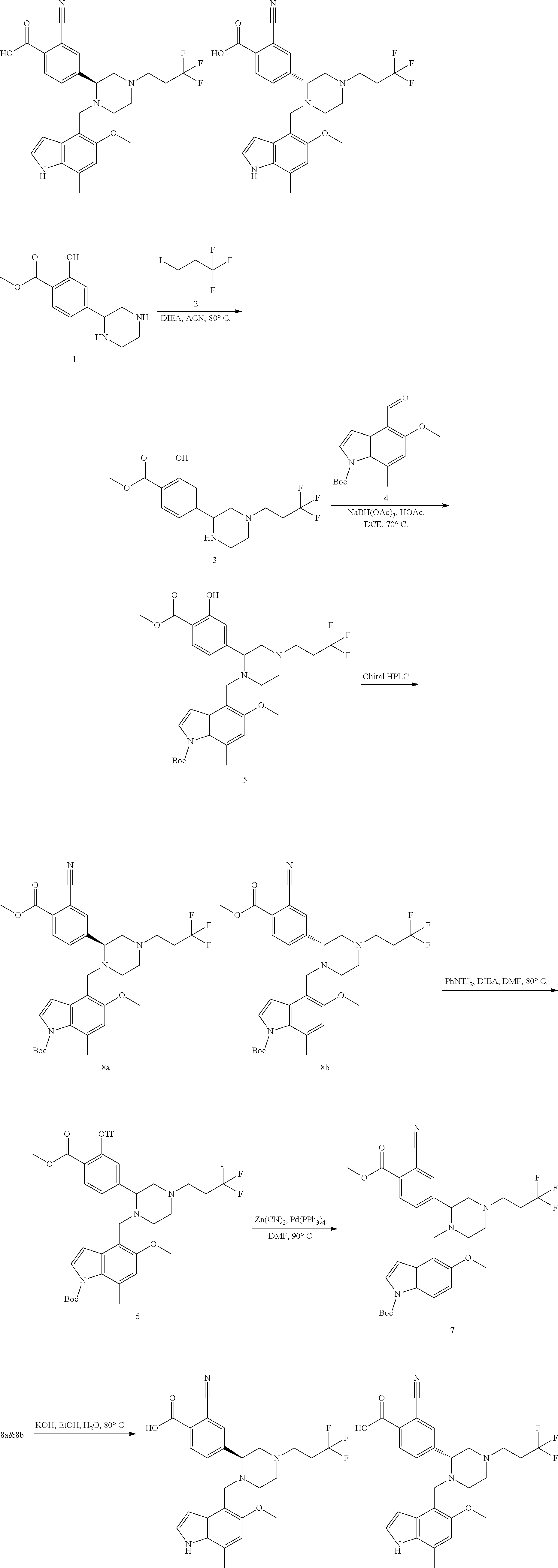

Step 1: Methyl 2-hydroxy-4-(4-(3,3,3-trifluoropropyl)piperazin-2-yl)benzoate. A solution of methyl 2-hydroxy-4-(piperazin-2-yl)benzoate (12.00 g, 50.79 mmol) in ACN (100 mL) was treated with 1,1,1-trifluoro-3-iodopropane (22.75 g, 101.58 mmol) and N,N-diisopropylethylamine (19.69 g, 152.37 mmol) for 18 h at 80° C. The solvent was removed. The residue was purified by reverse phase chromatography (Acetonitrile 0-40%/0.1% Ammonium bicarbonate in water) to afford the title compound (7.20 g, 42.7% yield) as a yellow oil. LCMS (ESI): [M+H]⁺=333.0.

Step 2: tert-Butyl 4-((2-(3-hydroxy-4-(methoxycarbonyl)phenyl)-4-(3,3,3-trifluoropropyl)piperazin-1-yl)methyl)-5-methoxy-7-methylindole-1-carboxylate. A solution of methyl 2-hydroxy-4-(4-(3,3,3-trifluoropropyl)piperazin-2-yl)benzoate (7.20 g, 21.67 mmol, 1 equiv) in dichloroethane (20 mL) was treated with tert-butyl 4-formyl-5-methoxy-7-methylindole-1-carboxylate (7.52 g, 25.99 mmol), acetic acid (1.30 g, 21.67 mmol) and Sodium triacetoxyborohydride (9.18 g, 43.33 mmol) at room temperature. The resulting mixture was stirred for overnight at 70° C. The solvent was concentrated under vacuum and the residue was purified by silica gel column eluting with petroleum ether/ethyl acetate (0-20%) to afford the title compound (7.50 g, 57.2% yield) as a yellow oil. LCMS (ESI): [M+H]⁺=606.0.

Step 3: tert-Butyl 5-methoxy-4-((2-(4-(methoxycarbonyl)-3-(trifluoromethanesulfonyloxy)phenyl)-4-(3,3,3-trifluoropropyl)piperazin-1-yl)methyl)-7-methylindole-1-carboxylate.

A solution of tert-butyl 4-((2-(3-hydroxy-4-(methoxycarbonyl)phenyl)-4-(3,3,3-trifluoropropyl)piperazin-1-yl)methyl)-5-methoxy-7-methylindole-1-carboxylate (6.50 g, 10.73 mmol) in DMF (100 mL) was treated with 1,1,1-trifluoro-N-phenyl-N-trifluoromethanesulfonylmethanesulfonamide (5.75 g, 16.10 mmol) and N,N-diisopropylethylamine (4.16 g, 32.20 mmol) and stirred for 2 h at 80° C. The reaction mixture was diluted with water and extracted with Ethyl acetate and the organic layers were combined. The organic layer was concentrated under vacuum and the residue was purified by silica gel column eluting with petroleum ether/ethyl acetate (0-10%) to afford the title compound (5.70 g, 71.9% yield) as a yellow oil. LCMS (ESI): [M+H]⁺=738.3.

Step 4: tert-Butyl 4-((2-(3-cyano-4-(methoxycarbonyl)phenyl)-4-(3,3,3-trifluoropropyl)piperazin-1-yl)methyl)-5-methoxy-7-methylindole-1-carboxylate.

A solution of tert-butyl 5-methoxy-4-((2-(4-(methoxycarbonyl)-3-(trifluoromethanesulfonyloxy)phenyl)-4-(3,3,3-trifluoropropyl)piperazin-1-yl)methyl)-7-methylindole-1-carboxylate (500 mg, 0.68 mmol), Zn(CN)₂ (159 mg, 1.36 mmol) and Pd(PPh₃)₄ (78 mg, 0.07 mmol) in DMF (4 mL) was stirred for 5 hours at 100° C. under nitrogen atmosphere. The reaction mixture was diluted with ethyl acetate and washed with water and the organic layers were combined. The organic layer was concentrated under vacuum. The residue was purified by silica gel column eluting with petroleum ether/ethyl acetate (0-15%) to afford the title compound (370 mg, 88.8% yield) as a yellow solid. LCMS (ESI): [M+H]⁺=615.3.

Step 5: tert-Butyl (S)-4-((2-(3-cyano-4-(methoxycarbonyl)phenyl)-4-(3,3,3-trifluoropropyl)piperazin-1-yl)methyl)-5-methoxy-7-methyl-1H-indole-1-carboxylate (isomer 1, faster peak)

tert-Butyl (R)-4-((2-(3-cyano-4-(methoxycarbonyl)phenyl)-4-(3,3,3-trifluoropropyl)piperazin-1-yl)methyl)-5-methoxy-7-methyl-1H-indole-1-carboxylate (isomer 2, slower peak) tert-butyl 4-((2-(3-cyano-4-(methoxycarbonyl)phenyl)-4-(3,3,3-trifluoropropyl)piperazin-1-yl)methyl)-5-methoxy-7-methylindole-1-carboxylate (150 mg) was applied for further separation by Chiral HPLC.

tert-Butyl (S)-4-((2-(3-cyano-4-(methoxycarbonyl)phenyl)-4-(3,3,3-trifluoropropyl)piperazin-1-yl)methyl)-5-methoxy-7-methyl-1H-indole-1-carboxylate (isomer 1, faster peak) (70 mg, 46.7% yield) LCMS (ESI): [M+H]⁺=615.3. tR=1.82 min (CHIRALPAK ID-3, 4.6*50 mm 3 μm), (Hex:DCM=3:1) (0.1% DEA):IPA=90:10, 1.0 mL/min, 254 nm). tert-Butyl (R)-4-((2-(3-cyano-4-(methoxycarbonyl)phenyl)-4-(3,3,3-trifluoropropyl)piperazin-1-yl)methyl)-5-methoxy-7-methyl-1H-indole-1-carboxylate (isomer 2, slower peak) (70 mg, 46.7% yield) LCMS (ESI): [M+H]⁺=615.3. tR=2.84 min (CHIRALPAK ID-3, 4.6*50 mm 3 μm), (Hex:DCM=3:1) (0.1% DEA):IPA=90:10, 1.0 mL/min, 254 nm).

Step 6: (S)-2-Cyano-4-(1-((5-methoxy-7-methyl-1H-indol-4-yl)methyl)-4-(3,3,3-trifluoropropyl)piperazin-2-yl)benzoic acid & (R)-2-cyano-4-(1-((5-methoxy-7-methyl-1H-indol-4-yl)methyl)-4-(3,3,3-trifluoropropyl)piperazin-2-yl)benzoic acid.

A solution of tert-butyl 4-(((2S)-2-(3-cyano-4-(methoxycarbonyl)phenyl)-4-(3,3,3-trifluoropropyl)piperazin-1-yl)methyl)-5-methoxy-7-methylindole-1-carboxylate (70 mg, 0.11 mmol) in Ethanol (2 mL) was added Potassium hydroxide (128 mg, 2.28 mmol) in water (0.4 mL) and stirred for 2 hours at 80° C. The mixture was allowed to cool down to room temperature and was neutralized to pH=6 with sat. Citric acid. The resulting residue was purified by reverse phase chromatography (Acetonitrile 0-30%/0.1% Ammonium bicarbonate in water) to afford 2-cyano-4-((2S)-1-((5-methoxy-7-methyl-1H-indol-4-yl)methyl)-4-(3,3,3-trifluoropropyl)piperazin-2-yl)benzoic acid (39.1 mg, 68.3% yield) as a white solid. LCMS (ESI, m/z): [M+H]⁺=501.2. ¹H NMR (300 MHz, DMSO-d₆) δ 10.85 (t, J=2.3 Hz, 1H), 8.20-8.00 (m, 2H), 7.96-7.81 (d, J=8.2 Hz, 1H), 7.26 (t, J=2.8 Hz, 1H), 6.65 (s, 1H), 6.40 (m, 1H), 3.73 (s, 3H), 3.58 (d, J=11.8 Hz, 1H), 3.53-3.44 (m, 2H), 3.25-3.16 (d, J=11.8 Hz, 2H), 2.87-2.59 (m, 3H), 2.47-2.43 (m, 2H), 2.41 (s, 3H), 2.30-2.20 (m, 1H), 2.07-1.93 (m, 2H).

A solution of tert-butyl 4-(((2S)-2-(3-cyano-4-(methoxycarbonyl)phenyl)-4-(3,3,3-trifluoropropyl)piperazin-1-yl)methyl)-5-methoxy-7-methylindole-1-carboxylate (70 mg, 0.11 mmol) in Ethanol (2 mL) was added Potassium hydroxide (128 mg, 2.28 mmol) in water (0.4 mL) and stirred for 2 hours at 80° C. The mixture was allowed to cool down to room temperature and was neutralized to pH=6 with sat. Citric acid. The resulting residue was purified by reverse phase chromatography (Acetonitrile 0-30%/0.1% Ammonium bicarbonate in water) to afford 2-cyano-4-((2S)-1-((5-methoxy-7-methyl-1H-indol-4-yl)methyl)-4-(3,3,3-trifluoropropyl)piperazin-2-yl)benzoic acid (39.5 mg, 69.1% yield) as a white solid. LCMS (ESI, m/z): [M+H]⁺=501.2. ¹H NMR (300 MHz, DMSO-d₆) δ 10.85 (t, J=2.3 Hz, 1H), 8.20-8.00 (m, 2H), 7.96-7.81 (d, J=8.2 Hz, 1H), 7.26 (t, J=2.8 Hz, 1H), 6.65 (s, 1H), 6.40 (m, 1H), 3.73 (s, 3H), 3.58 (d, J=11.8 Hz, 1H), 3.53-3.44 (m, 2H), 3.25-3.16 (d, J=11.8 Hz, 2H), 2.87-2.59 (m, 3H), 2.47-2.43 (m, 2H), 2.41 (s, 3H), 2.30-2.20 (m, 1H), 2.07-1.93 (m, 2H).

### Example 31. (S)-2-((Cyclopropylmethyl)amino)-4-(4-(2,2-difluoroethyl)-1-((5-methoxy-7-methyl-1H-indol-4-yl)methyl)piperazin-2-yl)benzoic acid & (R)-2-((cyclopropylmethyl)amino)-4-(4-(2,2-difluoroethyl)-1-((5-methoxy-7-methyl-1H-indol-4-yl)methyl)piperazin-2-yl)benzoic acid

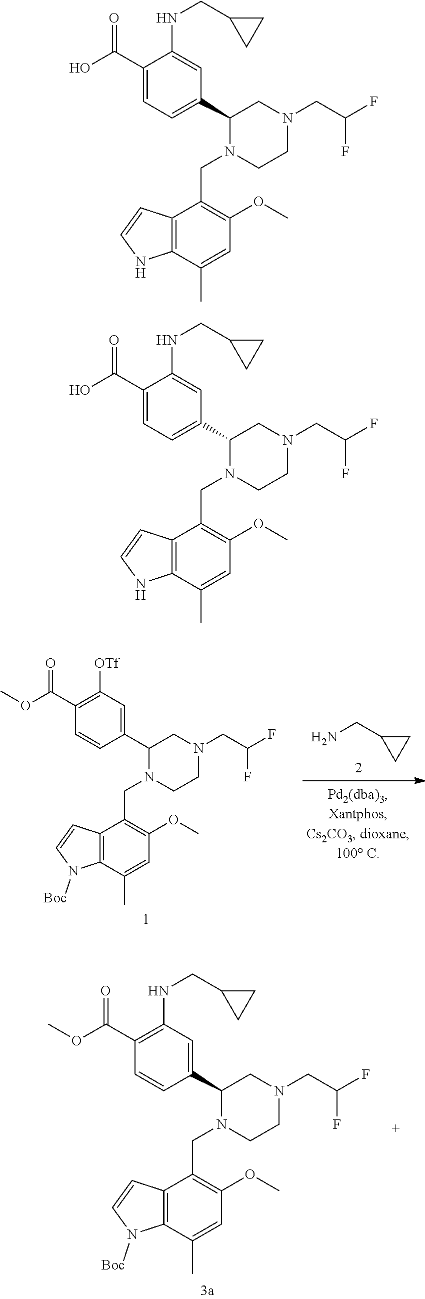

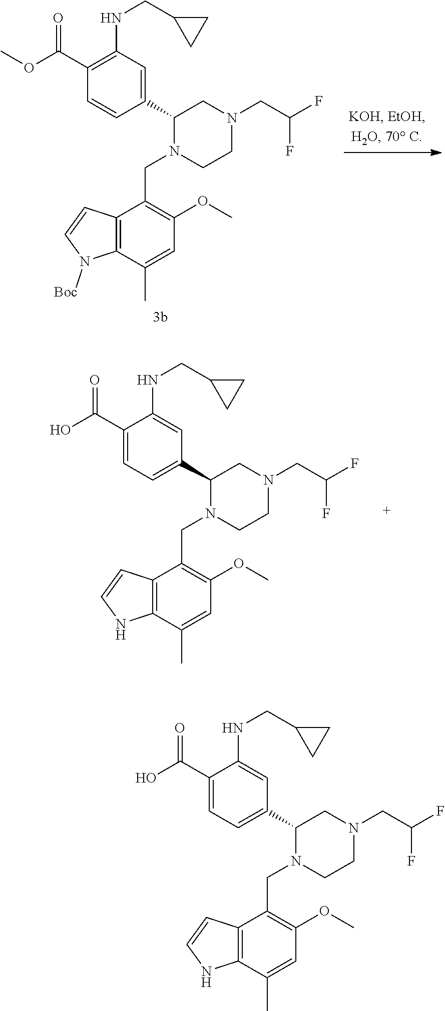 [SMILES: COC(=O)c1ccc([C@@H]2CN(CC(F)F)CCN2Cc2c(OC)cc(C)c3c2ccn3C(=O)OC(C)(C)C)cc1NCC1CC1 ; COc1cc(C)c2[nH]ccc2c1CN1CCN(CC(F)F)C[C@@H]1c1ccc(C(=O)O)c(NCC2CC2)c1 ; COc1cc(C)c2[nH]ccc2c1CN1CCN(CC(F)F)C[C@H]1c1ccc(C(=O)O)c(NCC2CC2)c1]

Step 1: tert-butyl (S)-4-((2-(3-((cyclopropylmethyl)amino)-4-(methoxycarbonyl)phenyl)-4-(2,2-difluoroethyl)piperazin-1-yl)methyl)-5-methoxy-7-methyl-1H-indole-1-carboxylate & tert-butyl (R)-4-((2-(3-((cyclopropylmethyl)amino)-4-(methoxycarbonyl)phenyl)-4-(2,2-difluoroethyl)piperazin-1-yl)methyl)-5-methoxy-7-methyl-1H-indole-1-carboxylate.

Under nitrogen, a mixture of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (400 mg, 0.56 mmol), potassium phosphate tribasic (360 mg, 1.69 mmol), tris(dibenzylideneacetone)dipalladium (52 mg, 0.05 mmol), XantPhos (66 mg, 0.11 mmol) and 1-cyclopropylmethanamine (194 mg, 2.72 mmol) in Toluene (4 mL) was stirred for 2 h at 110° C. After filtration, the filtrate was concentrated under reduced pressure. The residue was purified by reverse phase flash with ACN/10 mM NH₄HCO₃ in water and Chiral HPLC to afford the desired isomers.

tert-butyl (S)-4-((2-(3-((cyclopropylmethyl)amino)-4-(methoxycarbonyl)phenyl)-4-(2,2-difluoroethyl)piperazin-1-yl)methyl)-5-methoxy-7-methyl-1H-indole-1-carboxylate (94 mg, 26% yield, faster peak, isomer 1), LCMS (ESI): [M+H]⁺=627.2, tR=1.75 min (CHIRALPAK AD-3 4.6*50 mm 3 um, Hex(0.1% DEA):IPA=98:2, 1.0 mL/min, 254 nm).

tert-butyl (R)-4-((2-(3-((cyclopropylmethyl)amino)-4-(methoxycarbonyl)phenyl)-4-(2,2-difluoroethyl)piperazin-1-yl)methyl)-5-methoxy-7-methyl-1H-indole-1-carboxylate (108 mg, 30.4% yield, slower peak, isomer 2), LCMS (ESI): [M+H]⁺=627.2, tR=2.37 min (CHIRALPAK AD-3 4.6*50 mm 3 um, Hex(0.1% DEA):IPA=98:2, 1.0 mL/min, 254 nm).

Step 2: (S)-2-((Cyclopropylmethyl)amino)-4-(4-(2,2-difluoroethyl)-1-((5-methoxy-7-methyl-1H-indol-4-yl)methyl)piperazin-2-yl)benzoic acid & (R)-2-((cyclopropylmethyl)amino)-4-(4-(2,2-difluoroethyl)-1-((5-methoxy-7-methyl-1H-indol-4-yl)methyl)piperazin-2-yl)benzoic acid.

A mixture of tert-butyl (S)-4-((2-(3-((cyclopropylmethyl)amino)-4-(methoxycarbonyl)phenyl)-4-(2,2-difluoroethyl)piperazin-1-yl)methyl)-5-methoxy-7-methyl-1H-indole-1-carboxylate (94 mg, 0.15 mmol, faster peak, isomer 1) and potassium hydroxide (188 mg, 3.35 mmol) in water (1.5 mL) and ethanol (3 mL) was stirred for 4 h at 60° C. The mixture was neutralized to pH=7 with sat. citric acid. The crude product was purified by reverse phase (ACN/10 mM NH₄HCO₃ in water) to afford the title compound (40 mg, 53.2% yield) as a white solid. LCMS (ESI): [M+H]⁺=513.2. ¹H NMR (300 MHz, DMSO-d₆) δ 10.82 (s, 1H), 7.79 (d, J=8.1 Hz, 1H), 7.22 (t, J=2.8 Hz, 1H), 6.91-6.49 (m, 5H), 6.33-5.90 (m, 1H), 3.68 (s, 3H), 3.61 (d, J=12.0 Hz, 1H), 3.28-3.21 (m, 2H), 3.07 (d, J=6.9 Hz, 2H), 2.85-2.54 (m, 5H), 2.40 (s, 3H), 2.31-2.11 (m, 3H), 1.17-1.08 (m, 1H), 0.57-0.48 (m, 2H), 0.29-0.21 (m, 2H).

A mixture of tert-butyl (S)-4-((2-(3-((cyclopropylmethyl)amino)-4-(methoxycarbonyl)phenyl)-4-(2,2-difluoroethyl)piperazin-1-yl)methyl)-5-methoxy-7-methyl-1H-indole-1-carboxylate (98 mg, 0.15 mmol, slower peak, isomer 2) and potassium hydroxide (196 mg, 3.49 mmol) in water (1.5 mL) and ethanol (3 mL) was stirred for 4 h at 60° C. The mixture was neutralized to pH=7 with sat. citric acid. The crude product was purified by reverse phase (ACN/10 mM NH₄HCO₃ in water) to afford the title compound (31 mg, 39.6% yield) as a white solid. LCMS (ESI): [M+H]⁺=513.3. ¹H NMR (300 MHz, DMSO-d₆) δ 10.80 (s, 1H), 8.84-8.52 (m, 1H), 7.83 (s, 1H), 7.21 (t, J=2.8 Hz, 1H), 6.79-6.50 (m, 4H), 6.30-5.88 (m, 1H), 3.72-3.57 (m, 4H), 3.26-3.12 (m, 2H), 3.03 (d, J=6.7 Hz, 2H), 2.80-2.55 (m, 5H), 2.40 (s, 3H), 2.28 (t, J=10.6 Hz, 1H), 2.23-2.08 (m, 2H), 1.14-1.01 (m, 1H), 0.54 0.44 (m, 2H), 0.27-0.18 (m, 2H).

### Example 32. (S)-2-(Cyclobutylamino)-4-(4-(2,2-difluoroethyl)-1-((5-methoxy-7-methyl-1H-indol-4-yl)methyl)piperazin-2-yl)benzoic acid & (R)-2-(Cyclobutylamino)-4-(4-(2,2-difluoroethyl)-1-((5-methoxy-7-methyl-1H-indol-4-yl)methyl)piperazin-2-yl)benzoic acid

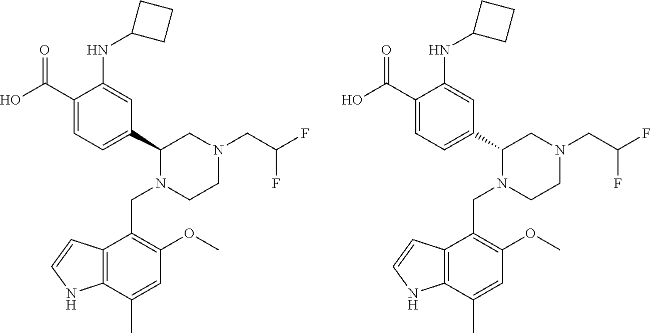 [SMILES: COc1cc(C)c2[nH]ccc2c1CN1CCN(CC(F)F)C[C@@H]1c1ccc(C(=O)O)c(NC2CCC2)c1 ; COc1cc(C)c2[nH]ccc2c1CN1CCN(CC(F)F)C[C@H]1c1ccc(C(=O)O)c(NC2CCC2)c1]

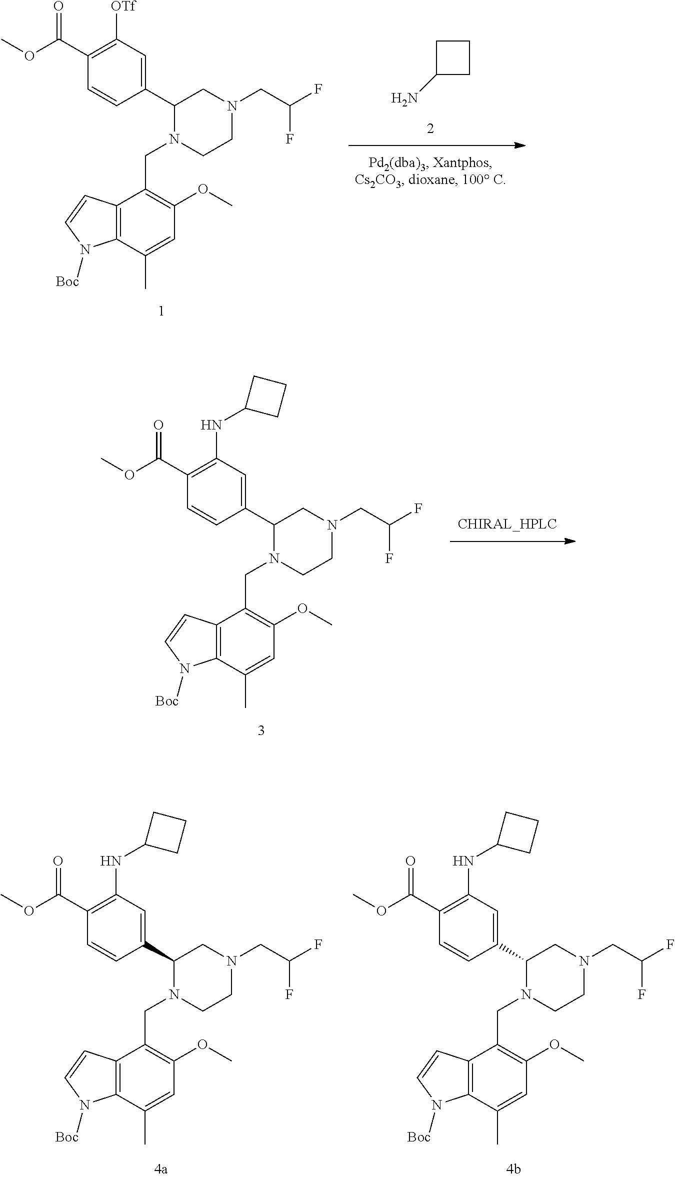 [SMILES: COC(=O)c1ccc(C2CN(CC(F)F)CCN2Cc2c(OC)cc(C)c3c2ccn3C(=O)OC(C)(C)C)cc1OS(=O)(=O)C(F)(F)F ; NC1CCC1 ; COC(=O)c1ccc(C2CN(CC(F)F)CCN2Cc2c(OC)cc(C)c3c2ccn3C(=O)OC(C)(C)C)cc1NC1CCC1 ; COC(=O)c1ccc([C@H]2CN(CC(F)F)CCN2Cc2c(OC)cc(C)c3c2ccn3C(=O)OC(C)(C)C)cc1NC1CCC1 ; COC(=O)c1ccc([C@@H]2CN(CC(F)F)CCN2Cc2c(OC)cc(C)c3c2ccn3C(=O)OC(C)(C)C)cc1NC1CCC1]

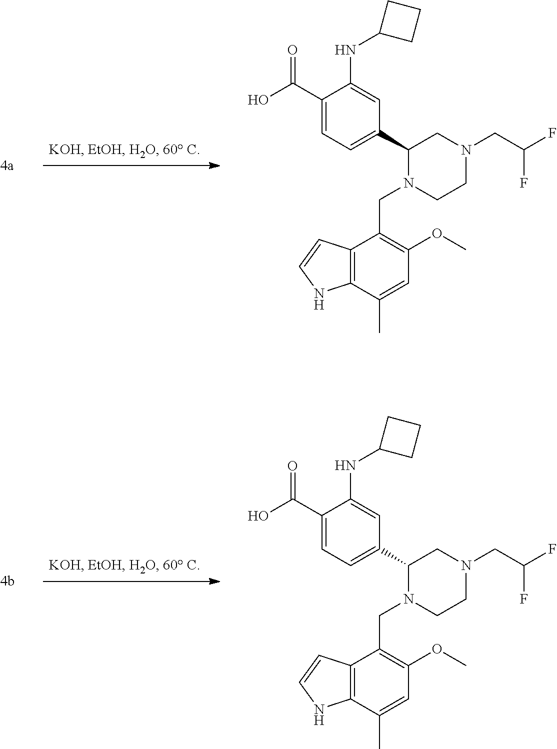 [SMILES: COc1cc(C)c2[nH]ccc2c1CN1CCN(CC(F)F)C[C@@H]1c1ccc(C(=O)O)c(NC2CCC2)c1 ; COc1cc(C)c2[nH]ccc2c1CN1CCN(CC(F)F)C[C@H]1c1ccc(C(=O)O)c(NC2CCC2)c1]

Step 1: tert-Butyl 4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate. To a stirred mixture of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (400 mg, 0.56 mmol), cyclobutanamine (398 mg, 5.6 mmol), Pd₂(dba)₃ (78 mg, 0.08 mmol), XantPhos (78 mg, 0.13 mmol) and Cesium carbonate (560 mg, 1.71 mmol) in 1,4-dioxane (5 mL) under nitrogen atmosphere. The resulting mixture was stirred for 1.5 h at 100° C. The solvent was concentrated under reduced pressure. The residue was purified by silica gel column chromatography, eluted with petroleum ether/ethyl acetate (4:1) to afford the title compound (230 mg, 64.7% yield) as a yellow solid. LCMS (ESI): [M+H]⁺=627.3.

Step 2: tert-Butyl (S)-4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (isomer 1, faster peak) & tert-Butyl (R)-4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (isomer 2, slower peak).

tert-Butyl 4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (230 mg) was applied for further separation by Chiral HPLC.

tert-Butyl (S)-4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (isomer 1, faster peak) (100 mg, 43.4% yield). t_{R}=1.61 min (CHIRALPAK AD-3, 4.6*50 mm 3 um, Hex(0.1% DEA):IPA=98:2, 1.0 mL/min, 254 nm).

tert-Butyl (R)-4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (isomer 2, slower peak) (90 mg, 39.1% yield). t_{R}=2.41 min (CHIRALPAK AD-3, 4.6*50 mm 3 um, Hex(0.1% DEA):IPA=98:2, 1.0 mL/min, 254 nm).

Step 3: (S)-2-(Cyclobutylamino)-4-(4-(2,2-difluoroethyl)-1-((5-methoxy-7-methyl-1H-indol-4-yl)methyl)piperazin-2-yl)benzoic acid & (R)-2-(Cyclobutylamino)-4-(4-(2,2-difluoroethyl)-1-((5-methoxy-7-methyl-1H-indol-4-yl)methyl)piperazin-2-yl)benzoic acid.

To a stirred mixture of tert-Butyl (S)-4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (100 mg, 0.15 mmol) and Potassium hydroxide (100 mg, 1.78 mmol) in Ethanol (3 mL) and water (0.5 mL) was stirred for 3 h at 60° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. to afford (S)-2-(Cyclobutylamino)-4-(4-(2,2-difluoroethyl)-1-((5-methoxy-7-methyl-1H-indol-4-yl)methyl)piperazin-2-yl)benzoic acid (40.1 mg, 49.3% yield) as a white solid. LCMS (ESI): [M+H]⁺=513.3. ¹H NMR (300 MHz, Methanol-d₄) δ 7.98 (d, J=8.0 Hz, 1H), 7.25 (d, J=3.1 Hz, 1H), 6.82-6.71 (m, 3H), 6.37 (d, J=3.1 Hz, 1H), 5.97 (tt, J=55.8, 4.2 Hz, 1H), 4.19 (d, J=12.4 Hz, 1H), 4.14-4.05 (m, 1H), 4.04-3.93 (m, 1H), 3.83 (d, J=12.4 Hz, 1H), 3.18 (d, J=12.0 Hz, 1H), 3.10-2.92 (m, 3H), 2.92-2.59 (m, 4H), 2.56-2.41 (m, 5H), 2.05-1.75 (m, 4H).

To a stirred mixture of tert-Butyl (R)-4-((2-(3-(cyclobutylamino)-4-(methoxycarbonyl)phenyl)-4-(2,2-difluoroethyl)piperazin-1-yl)methyl)-5-methoxy-7-methyl-1H-indole-1-carboxylate (98 mg, 0.15 mmol) and Potassium hydroxide (100 mg, 1.78 mmol) in Ethanol (3 mL) and water (0.5 mL). The resulting mixture was stirred for 3 h at 60° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH₄HCO₃), 10% to 50% gradient in 30 min; detector, UV 254 nm. to afford (R)-2-(Cyclobutylamino)-4-(4-(2,2-difluoroethyl)-1-((5-methoxy-7-methyl-1H-indol-4-yl)methyl)piperazin-2-yl)benzoic acid (38.6 mg, 54.7% yield) as a white solid. LCMS (ESI): [M+H]⁺=513.3. ¹H NMR (300 MHz, Methanol-d₄) δ 7.98 (d, J=8.0 Hz, 1H), 7.25 (d, J=3.1 Hz, 1H), 6.82-6.71 (m, 3H), 6.37 (d, J=3.1 Hz, 1H), 5.97 (tt, J=55.8, 4.2 Hz, 1H), 4.19 (d, J=12.4 Hz, 1H), 4.14-4.05 (m, 1H), 4.04-3.93 (m, 1H), 3.83 (d, J=12.4 Hz, 1H), 3.18 (d, J=12.0 Hz, 1H), 3.10-2.92 (m, 3H), 2.92-2.59 (m, 4H), 2.56-2.41 (m, 5H), 2.05-1.75 (m, 4H).

### Example 33. 4-((2S)-4-(2,2-Difluoroethyl)-1-((5-methoxy-7-methyl-1H-indol-4-yl)methyl)piperazin-2-yl)-2-((2,2,2-trifluoroethyl)amino)benzoic acid & 4-((2R)-4-(2,2-difluoroethyl)-1-((5-methoxy-7-methyl-1H-indol-4-yl)methyl)piperazin-2-yl)-2-((2,2,2-trifluoroethyl)amino)benzoic acid

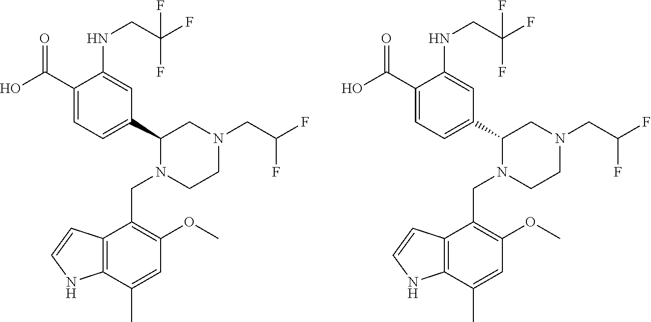 [SMILES: COc1cc(C)c2[nH]ccc2c1CN1CCN(CC(F)F)C[C@@H]1c1ccc(C(=O)O)c(NCC(F)(F)F)c1 ; COc1cc(C)c2[nH]ccc2c1CN1CCN(CC(F)F)C[C@H]1c1ccc(C(=O)O)c(NCC(F)(F)F)c1]

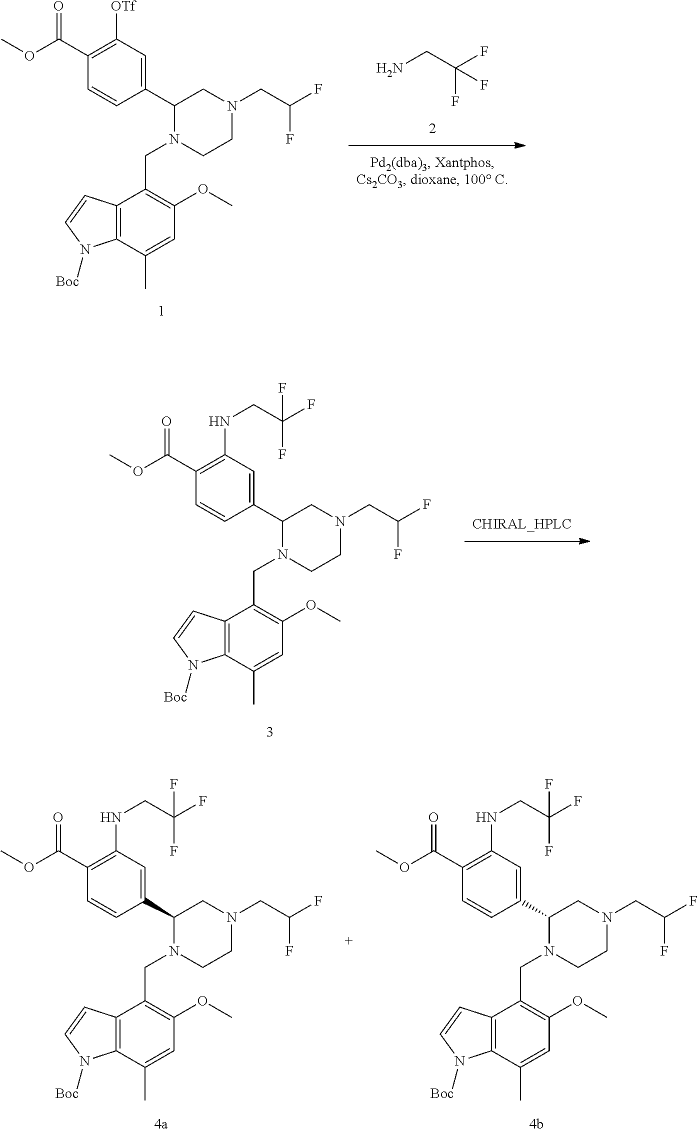 [SMILES: COC(=O)c1ccc(C2CN(CC(F)F)CCN2Cc2c(OC)cc(C)c3c2ccn3C(=O)OC(C)(C)C)cc1OS(=O)(=O)C(F)(F)F ; NCC(F)(F)F ; COC(=O)c1ccc(C2CN(CC(F)F)CCN2Cc2c(OC)cc(C)c3c2ccn3C(=O)OC(C)(C)C)cc1NCC(F)(F)F ; COC(=O)c1ccc([C@H]2CN(CC(F)F)CCN2Cc2c(OC)cc(C)c3c2ccn3C(=O)OC(C)(C)C)cc1NCC(F)(F)F ; COC(=O)c1ccc([C@@H]2CN(CC(F)F)CCN2Cc2c(OC)cc(C)c3c2ccn3C(=O)OC(C)(C)C)cc1NCC(F)(F)F]

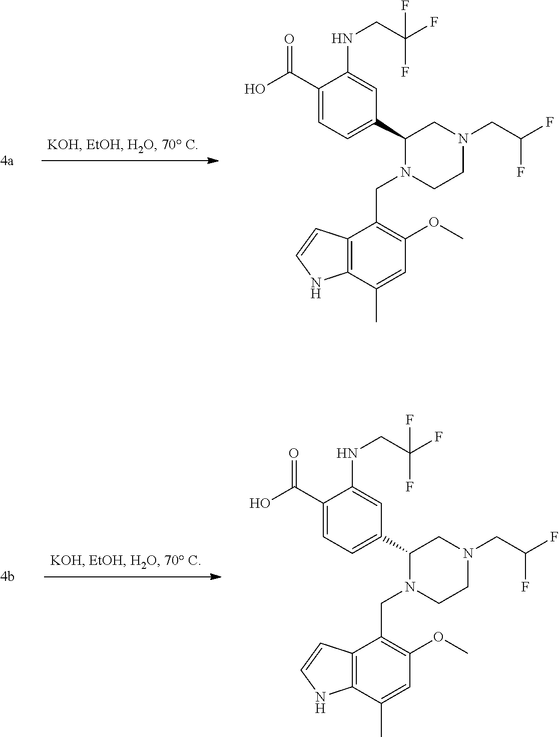 [SMILES: COc1cc(C)c2[nH]ccc2c1CN1CCN(CC(F)F)C[C@@H]1c1ccc(C(=O)O)c(NCC(F)(F)F)c1 ; COc1cc(C)c2[nH]ccc2c1CN1CCN(CC(F)F)C[C@H]1c1ccc(C(=O)O)c(NCC(F)(F)F)c1]

Step 1: tert-Butyl 4-((4-(2,2-difluoroethyl)-2-(4-(methoxycarbonyl)-3-((2,2,2-trifluoroethyl)amino)phenyl]piperazin-1-yl)methyl)-5-methoxy-7-methylindole-1-carboxylate. To a mixture of tert-butyl 4-((4-(2,2-difluoroethyl)-2-(4-(methoxycarbonyl)-3-(trifluoromethanesulfonyloxy)phenyl)piperazin-1-yl)methyl)-5-methoxy-7-methylindole-1-carboxylate (200 mg, 0.28 mmol), 2,2,2-trifluoroethylamine (140 mg, 1.41 mmol), Cs₂CO₃ (280 mg, 0.86 mmol), Xantphos (30 mg, 0.05 mmol) and Pd₂(dba)₃ (30 mg, 0.03 mmol) in 1,4-dioxane (4 mL) was stirred for 2 h at 100° C. under nitrogen atmosphere. The resulting mixture was extracted with ethyl acetate. The combined organic layers were washed with brine, dried over anhydrous Na₂SO₄. After filtration, the filtrate was concentrated under reduced pressure. The residue was purified by silica gel column chromatography, eluted with petroleum ether/ethyl acetate (1:1) to afford the title compound (110 mg, 59.14% yield) as a yellow solid. LCMS (ESI): [M+H]⁺=655.2.

Step 2: tert-butyl 4-(((2S)-4-(2,2-difluoroethyl)-2-(4-(methoxycarbonyl)-3-((2,2,2-trifluoroethyl)amino)phenyl)piperazin-1-yl)methyl)-5-methoxy-7-methylindole-1-carboxylate (isomer 1, faster peak) & tert-butyl 4-(((2R)-4-(2,2-difluoroethyl)-2-(4-(methoxycarbonyl)-3-((2,2,2-trifluoroethyl)amino)phenyl)piperazin-1-yl)methyl)-5-methoxy-7-methylindole-1-carboxylate (isomer 2, slower peak).

The tert-butyl 4-((4-(2,2-difluoroethyl)-2-(4-(methoxycarbonyl)-3-((2,2,2-trifluoroethyl)amino)phenyl]piperazin-1-yl)methyl)-5-methoxy-7-methylindole-1-carboxylate (110 mg) was applied for further separation by Chiral HPLC with the folloing condition: Column: CHIRAL ART Cellulose-SB, 2*25 cm, 5 μm; Mobile Phase A: Hex(0.1% DEA)-HPLC, Mobile Phase B: Ethanol-HPLC; Flow rate: 20 mL/min; Gradient: 2% B to 2% B in 8 min; Wave Length: 220/254 nm; RT1(min): 5.583; RT2(min): 7.408; Sample Solvent: Ethanol-HPLC; Injection Volume: 0.5 mL; Number of Runs: 6.

tert-Butyl 4-(((2S)-4-(2,2-difluoroethyl)-2-(4-(methoxycarbonyl)-3-((2,2,2-trifluoroethyl)amino)phenyl)piperazin-1-yl)methyl)-5-methoxy-7-methylindole-1-carboxylate (isomer 1, faster peak) (45 mg, 40.91% yield) as a white solid. LCMS (ESI): [M+H]⁺=655.2. t_{R}=2.31 min, (CHIRAL Cellulose-SB (4.6*100 mm, 3 μm), Hex(0.1% DEA):Ethanol=98:2, 1.0 mL/min, 254 nm).

tert-butyl 4-(((2R)-4-(2,2-difluoroethyl)-2-(4-(methoxycarbonyl)-3-((2,2,2-trifluoroethyl)amino)phenyl)piperazin-1-yl)methyl)-5-methoxy-7-methylindole-1-carboxylate (isomer 2, slower peak) (46 mg, 41.82% yield) as a light yellow solid. LCMS (ESI): [M+H]⁺=655.2. t_{R}=2.93 min, (CHIRAL Cellulose-SB (4.6*100 mm, 3 μm), Hex(0.1% DEA):Ethanol=98:2, 1.0 mL/min, 254 nm).

Step 3: 4-((2S)-4-(2,2-Difluoroethyl)-1-((5-methoxy-7-methyl-1H-indol-4-yl)methyl)piperazin-2-yl)-2-((2,2,2-trifluoroethyl)amino)benzoic acid & 4-((2R)-4-(2,2-difluoroethyl)-1-((5-methoxy-7-methyl-1H-indol-4-yl)methyl)piperazin-2-yl)-2-((2,2,2-trifluoroethyl)amino)benzoic acid.

To a mixture of tert-butyl 4-(((2S)-4-(2,2-difluoroethyl)-2-(4-(methoxycarbonyl)-3-((2,2,2-trifluoroethyl)amino)phenyl)piperazin-1-yl)methyl)-5-methoxy-7-methylindole-1-carboxylate (45 mg, 0.07 mmol) and Potassium hydroxide (40 mg, 0.71 mmol) in water (1 mL) and Ethanol (2 mL) was stirred for 1 h at 60° C. The mixture was allowed to cool down to room temperature. The mixture was acidified to pH=6 with sat. citric acid. The residue was purified by reverse phase flash with the following conditions (acetonitrile, 5-35%/10 mM NH₄HCO₃ in water) to afford 4-((2S)-4-(2,2-difluoroethyl)-1-((5-methoxy-7-methyl-1H-indol-4-yl)methyl)piperazin-2-yl)-2-((2,2,2-trifluoroethyl)amino)benzoic acid (23.9 mg, 64.26% yield) as a white solid. LCMS (ESI): [M+H]⁺=541.2; ¹H NMR (300 MHz, DMSO-d₆) δ 12.86 (s, 1H), 10.84 (s, 1H), 8.31-8.14 (m, 1H), 7.87 (d, J=8.1 Hz, 1H), 7.20 (d, J=27.8 Hz, 2H), 7.05-6.85 (m, 1H), 6.66 (s, 1H), 6.50 (s, 1H), 6.34-5.89 (m, 1H), 4.40-4.09 (m, 2H), 3.70 (s, 3H), 3.61 (d, J=12.3 Hz, 1H), 3.31-3.17 (m, 2H), 2.98-2.57 (m, 5H), 2.42 (s, 3H), 2.28 (t, J=11.0 Hz, 1H), 2.18 (d, J=7.8 Hz, 2H).

To a mixture of tert-butyl 4-(((2R)-4-(2,2-difluoroethyl)-2-(4-(methoxycarbonyl)-3-((2,2,2-trifluoroethyl)amino)phenyl)piperazin-1-yl)methyl)-5-methoxy-7-methylindole-1-carboxylate (46 mg, 0.07 mmol) and Potassium hydroxide (41 mg, 0.73 mmol) in water (1 mL) and Ethanol (2 mL) was stirred for 1 h at 60° C. The mixture was allowed to cool down to room temperature. The mixture was acidified to pH=6 with sat. citric acid. The residue was purified by reverse phase flash with the following conditions (acetonitrile, 5-35%/10 mM NH₄HCO₃ in water) to afford 4-((2R)-4-(2,2-difluoroethyl)-1-((5-methoxy-7-methyl-1H-indol-4-yl)methyl)piperazin-2-yl)-2-((2,2,2-trifluoroethyl)amino)benzoic acid (20.5 mg, 53.92%) as a white solid. (25.4 mg, 58.94% yield) as a white solid. LCMS (ESI): [M+H]⁺=541.2; ¹H NMR (300 MHz, DMSO-d₆) δ 12.85 (s, 1H), 10.84 (s, 1H), 8.23 (t, J=6.8 Hz, 1H), 7.87 (d, J=8.2 Hz, 1H), 7.20 (d, J=27.6 Hz, 2H), 7.04-6.87 (m, 1H), 6.66 (s, 1H), 6.50 (s, 1H), 6.34-5.89 (m, 1H), 4.41-4.07 (m, 2H), 3.70 (s, 3H), 3.61 (d, J=12.1 Hz, 1H), 3.31-3.19 (m, 2H), 2.94 2.55 (m, 5H), 2.42 (s, 3H), 2.28 (t, J=10.8 Hz, 1H), 2.18 (d, J=8.0 Hz, 2H).

### Example 34. (S)-4-(4-(2,2-Difluoroethyl)-1-((5-methoxy-7-methyl-1H-indol-4-yl)methyl)piperazin-2-yl)-2-((oxetan-3-ylmethyl)amino)benzoic acid & (R)-4-(4-(2,2-difluoroethyl)-1-((5-methoxy-7-methyl-1H-indol-4-yl)methyl)piperazin-2-yl)-2-((oxetan-3-ylmethyl)amino)benzoic acid

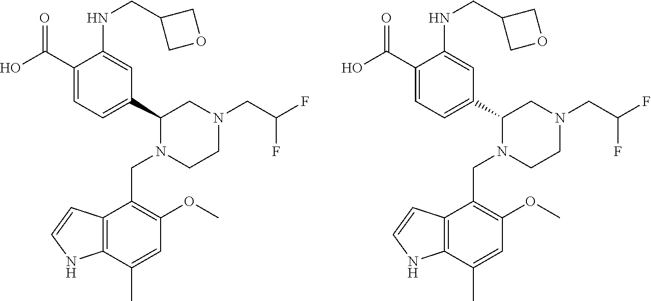 [SMILES: COc1cc(C)c2[nH]ccc2c1CN1CCN(CC(F)F)C[C@@H]1c1ccc(C(=O)O)c(NCC2COC2)c1 ; COc1cc(C)c2[nH]ccc2c1CN1CCN(CC(F)F)C[C@H]1c1ccc(C(=O)O)c(NCC2COC2)c1]

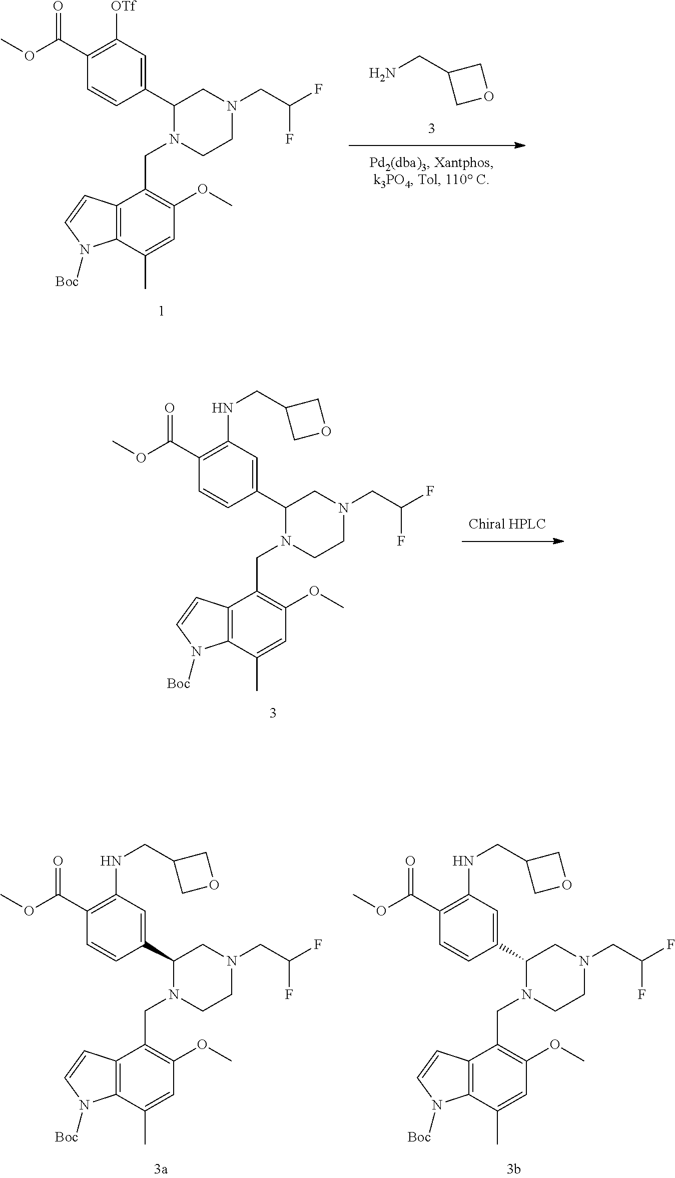 [SMILES: COC(=O)c1ccc(C2CN(CC(F)F)CCN2Cc2c(OC)cc(C)c3c2ccn3C(=O)OC(C)(C)C)cc1OS(=O)(=O)C(F)(F)F ; NCC1COC1 ; COC(=O)c1ccc(C2CN(CC(F)F)CCN2Cc2c(OC)cc(C)c3c2ccn3C(=O)OC(C)(C)C)cc1NCC1COC1 ; COC(=O)c1ccc([C@H]2CN(CC(F)F)CCN2Cc2c(OC)cc(C)c3c2ccn3C(=O)OC(C)(C)C)cc1NCC1COC1 ; COC(=O)c1ccc([C@@H]2CN(CC(F)F)CCN2Cc2c(OC)cc(C)c3c2ccn3C(=O)OC(C)(C)C)cc1NCC1COC1]

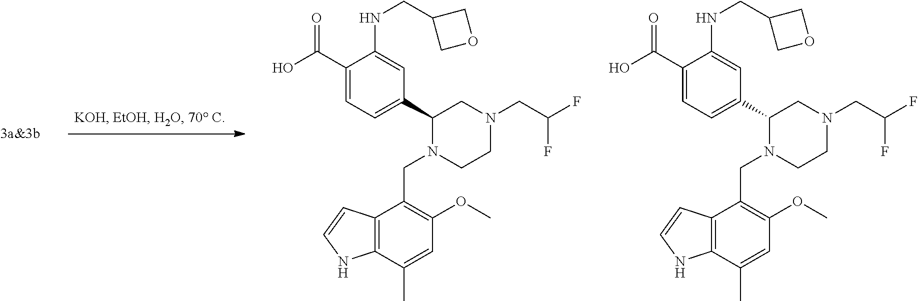 [SMILES: COc1cc(C)c2[nH]ccc2c1CN1CCN(CC(F)F)C[C@@H]1c1ccc(C(=O)O)c(NCC2COC2)c1 ; COc1cc(C)c2[nH]ccc2c1CN1CCN(CC(F)F)C[C@H]1c1ccc(C(=O)O)c(NCC2COC2)c1]

Step 1: tert-Butyl 4-((4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate. A solution of tert-butyl 4-((4-(2,2-difluoroethyl)-2-(4-(methoxycarbonyl)-3-(trifluoromethanesulfonyloxy)phenyl)piperazin-1-yl)methyl)-5-methoxy-7-methylindole-1-carboxylate (250 mg, 0.35 mmol) in Toluene (4 mL) was added 1-(oxetan-3-yl)methanamine (154 mg, 1.77 mmol), XantPhos (41 mg, 0.07 mmol), Pd₂(dba)₃ (32 mg, 0.03 mmol) and K₃PO₄ (150 mg, 0.71 mmol) and stirred for 2 hours at 110° C. under nitrogen atmosphere. The solvent was removed under vacuum and the residue was purified by silica gel column eluting with petroleum ether/ethyl acetate (0-16%) to afford the title compound (220 mg, 96.6% yield) as a yellow oil. LCMS (ESI): [M+H]⁺=643.0.

Step 2: tert-butyl 4-(((2S)-4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (isomer 1, faster peak).

tert-butyl 4-(((2R)-4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (isomer 2, slower peak).

tert-Butyl 4-((4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (220 mg) was applied for separation by Chiral HPLC.

tert-butyl 4-(((2S)-4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (isomer 1, faster peak) (70 mg, 31.8% yield), LCMS (ESI): [M+H]⁺=643.0. tR=2.52 min (CHIRALPAK IC-3, 4.6*50 mm 3 μm), Hex(0.1% DEA):IPA=80:20, 1.0 mL/min, 254 nm).

tert-butyl 4-(((2R)-4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (isomer 2, slower peak) (80 mg, 36.3% yield), LCMS (ESI): [M+H]⁺=643.0. tR=3.29 min (CHIRALPAK IC-3, 4.6*50 mm 3 μm), Hex(0.1% DEA):IPA=80:20, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-((oxetan-3-ylmethyl)amino)benzoic acid & (R)-4-(4-(2,2-difluoroethyl)-1-((5-methoxy-7-methyl-1H-indol-4-yl)methyl)piperazin-2-yl)-2-((oxetan-3-ylmethyl)amino)benzoic acid.

A solution of tert-butyl 4-(((2S)-4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (70 mg, 0.10 mmol) in Ethanol (2 mL) was added Potassium hydroxide (122 mg, 2.18 mmol) in water (0.4 mL) and stirred for 1.5 hours at 80° C. The mixture was allowed to cool down to room temperature and was acid to pH=7 with sat. citric acid. The resulting residue was purified by reverse phase chromatography (Acetonitrile 0-30%/0.1% Ammonium bicarbonate in water) to afford the title compound (46.4 mg, 79.6% yield) as a white solid. LCMS (ESI): [M+H]⁺=529.3. ¹H NMR (300 MHz, DMSO-d₆) δ 10.82 (s, 1H), 7.79 (d, J=8.1 Hz, 1H), 7.24 (t, J=2.8 Hz, 1H), 6.93 (s, 1H), 6.77 (s, 1H), 6.65 (s, 1H), 6.54 (d, J=2.9 Hz, 1H), 6.31-5.89 (m, 1H), 4.75-4.57 (m, 2H), 4.38-4.25 (m, 2H), 3.69 (s, 4H), 3.52 (d, J=7.2 Hz, 2H), 3.24 (d, J=6.1 Hz, 3H), 2.86-2.54 (m, 6H), 2.41 (s, 3H), 2.28 (t, J=10.7 Hz, 1H), 2.18 (s, 2H).

A solution of tert-butyl 4-(((2R)-4-(2,2-difluoroethyl)-2-(4-(methoxycarbonyl)-3-((oxetan-3-ylmethyl)amino)phenyl)piperazin-1-yl)methyl)-5-methoxy-7-methylindole-1-carboxylate (70 mg, 0.10 mmol) in Ethanol (2 mL) was added Potassium hydroxide (122 mg, 2.18 mmol) in water (0.4 mL) and stirred for 1.5 hours at 80° C. The mixture was allowed to cool down to room temperature and was acid to pH=7 with sat. citric acid. The resulting residue was purified by reverse phase chromatography (Acetonitrile 0-30%/0.1% Ammonium bicarbonate in water) to afford the title compound (47.4 mg, 81.5% yield) as a white solid. LCMS (ESI): [M+H]⁺=529.3. ¹H NMR (300 MHz, DMSO-d₆) δ 10.82 (s, 1H), 7.79 (d, J=8.1 Hz, 1H), 7.24 (t, J=2.8 Hz, 1H), 6.93 (s, 1H), 6.77 (s, 1H), 6.65 (s, 1H), 6.54 (d, J=2.9 Hz, 1H), 6.31-5.89 (m, 1H), 4.75-4.57 (m, 2H), 4.38-4.25 (m, 2H), 3.69 (s, 4H), 3.52 (d, J=7.2 Hz, 2H), 3.24 (d, J=6.1 Hz, 3H), 2.86-2.54 (m, 6H), 2.41 (s, 3H), 2.28 (t, J=10.7 Hz, 1H), 2.18 (s, 2H).

### Example 35. (S)-4-(4-(2,2-Difluoroethyl)-1-((5-methoxy-7-methyl-1H-indol-4-yl)methyl)piperazin-2-yl)-2-((tetrahydro-2H-pyran-4-yl)amino)benzoic acid & (R)-4-(4-(2,2-difluoroethyl)-1-((5-methoxy-7-methyl-1H-indol-4-yl)methyl)piperazin-2-yl)-2-((tetrahydro-2H-pyran-4-yl)amino)benzoic acid

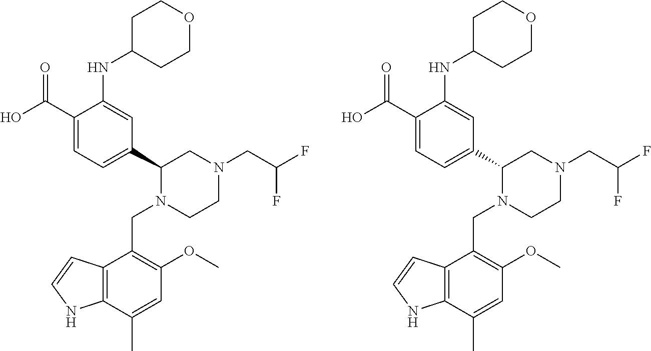 [SMILES: COc1cc(C)c2[nH]ccc2c1CN1CCN(CC(F)F)C[C@@H]1c1ccc(C(=O)O)c(NC2CCOCC2)c1 ; COc1cc(C)c2[nH]ccc2c1CN1CCN(CC(F)F)C[C@H]1c1ccc(C(=O)O)c(NC2CCOCC2)c1]

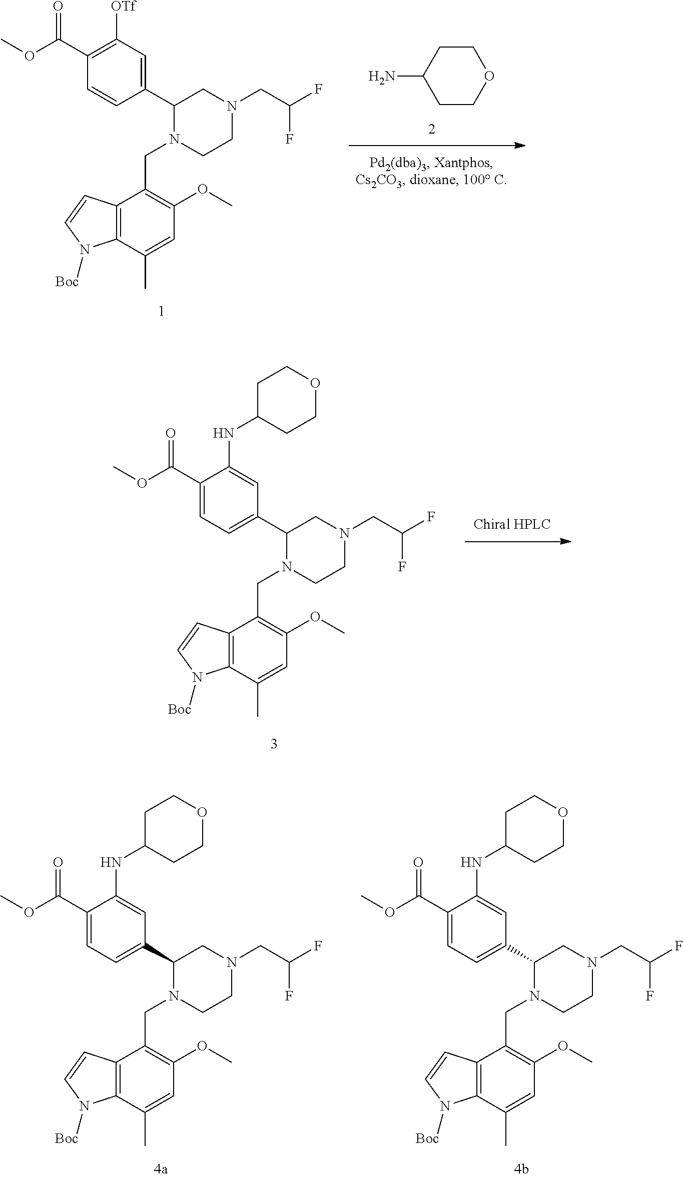 [SMILES: COC(=O)c1ccc(C2CN(CC(F)F)CCN2Cc2c(OC)cc(C)c3c2ccn3C(=O)OC(C)(C)C)cc1OS(=O)(=O)C(F)(F)F ; NC1CCOCC1 ; COC(=O)c1ccc(C2CN(CC(F)F)CCN2Cc2c(OC)cc(C)c3c2ccn3C(=O)OC(C)(C)C)cc1NC1CCOCC1 ; COC(=O)c1ccc([C@H]2CN(CC(F)F)CCN2Cc2c(OC)cc(C)c3c2ccn3C(=O)OC(C)(C)C)cc1NC1CCOCC1 ; COC(=O)c1ccc([C@@H]2CN(CC(F)F)CCN2Cc2c(OC)cc(C)c3c2ccn3C(=O)OC(C)(C)C)cc1NC1CCOCC1]

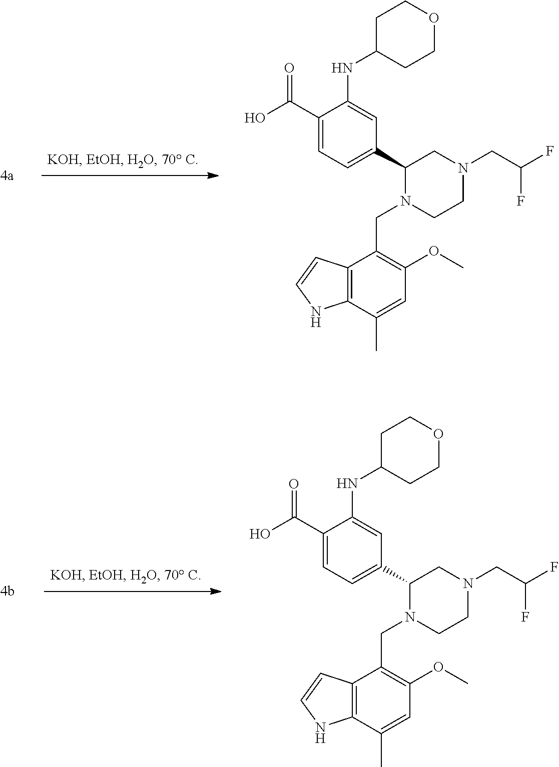 [SMILES: COc1cc(C)c2[nH]ccc2c1CN1CCN(CC(F)F)C[C@@H]1c1ccc(C(=O)O)c(NC2CCOCC2)c1 ; COc1cc(C)c2[nH]ccc2c1CN1CCN(CC(F)F)C[C@H]1c1ccc(C(=O)O)c(NC2CCOCC2)c1]

Step 1: tert-Butyl 4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate. Under nitrogen, a solution of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (500 mg, 0.71 mmol) and oxan-4-amine (215 mg, 2.12 mmol), tris(dibenzylideneacetone)dipalladium (130 mg, 0.14 mmol), XantPhos (123 mg, 0.21 mmol) and cesium carbonate (695 mg, 2.13 mmol) in dioxane (5 mL) was stirred for 2 h at 100° C. The solvent was removed under vacuum. The residue was purified by silica gel column chromatography, eluted with ethyl acetate/petroleum ether (3/10) to afford the title compound (300 mg, 64.5% yield) as a yellow solid. LCMS (ESI) [M+H]⁺=667.34.

Step 2: tert-Butyl (S)-4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate (isomer 1, slower peak) & tert-butyl (R)-4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate (isomer 2, faster peak).

tert-Butyl 4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate (300 mg) was applied for further separation by Chiral HPLC.

tert-Butyl (S)-4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate (130 mg, 43.3% yield, isomer 1, faster peak), LCMS (ESI) [M+H]⁺=567.3. tR=1.49 min (CHIRALPAK IG-3, 4.6*50 mm, 3 μm, Hex(0.1% DEA):Ethanol=90:10, 1.0 mL/min, 254 nm).

tert-Butyl (R)-4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate (120 mg, 40.0% yield, isomer 2, slower peak). LCMS (ESI) [M+H]⁺=567.3. tR=1.87 min (CHIRALPAK IG-3, 4.6*50 mm, 3 μm, Hex(0.1% DEA):Ethanol=90:10, 1.0 mL/min, 254 nm).

Step 3: (S)-4-(4-(2,2-Difluoroethyl)-1-((5-methoxy-7-methyl-1H-indol-4-yl)methyl)piperazin-2-yl)-2-((tetrahydro-2H-pyran-4-yl)amino)benzoic acid & (R)-4-(4-(2,2-difluoroethyl)-1-((5-methoxy-7-methyl-1H-indol-4-yl)methyl)piperazin-2-yl)-2-((tetrahydro-2H-pyran-4-yl)amino)benzoic acid.

A solution of tert-Butyl (S)-4-((4-(2,2-difluoroethyl)-2-(4-(methoxycarbonyl)-3-((tetrahydro-2H-pyran-4-yl)amino)phenyl)piperazin-1-yl)methyl)-5-methoxy-7-methyl-1H-indole-1-carboxylate (130 mg, 0.23 mmol) in ethanol (2.5 mL) and water (0.5 mL) was added potassium hydroxide (128 mg, 2.3 mmol) and stirred for 3 h at 70° C. The reaction mixture was acidified to pH=6 with sat. citric acid solution. The solvent was removed under vacuum. The residue was purified by reversed-phase flash chromatography with MeCN/10 mmol/L NH₄HCO₃) to afford the title compound.

(S)-4-(4-(2,2-Difluoroethyl)-1-((5-methoxy-7-methyl-1H-indol-4-yl)methyl)piperazin-2-yl)-2-((tetrahydro-2H-pyran-4-yl)amino)benzoic acid (40.7 mg, 37.8% yield) as a white solid. LCMS (ESI) [M+H]⁺=543.2. ¹H NMR (300 MHz, Methanol-d₄) δ 7.99 (d, J=8.1 Hz, 1H), 7.24 (d, J=3.1 Hz, 1H), 6.97 (s, 1H), 6.73 (s, 2H), 6.40 (d, J=3.1 Hz, 1H), 6.20-5.73 (m, 1H), 4.17 (d, J=12.6 Hz, 1H), 4.02-3.88 (m, 3H), 3.77 (s, 3H), 3.72-3.70 (m, 1H), 3.61-3.44 (m, 2H), 3.34 (s, 1H), 3.17-3.13 (m, 1H), 3.02-2.90 (m, 3H), 2.88-2.73 (m, 2H), 2.71-2.53 (m, 2H), 2.48 (d, J=0.8 Hz, 3H), 2.07-2.03 (m, 2H), 1.63-1.44 (m, 2H).

(R)-4-(4-(2,2-Difluoroethyl)-1-((5-methoxy-7-methyl-1H-indol-4-yl)methyl)piperazin-2-yl)-2-((tetrahydro-2H-pyran-4-yl)amino)benzoic acid was prepared according to above.

(R)-4-(4-(2,2-Difluoroethyl)-1-((5-methoxy-7-methyl-1H-indol-4-yl)methyl)piperazin-2-yl)-2-((tetrahydro-2H-pyran-4-yl)amino)benzoic acid (39.9 mg, 40.2% yield) as a white solid. LCMS (ESI) [M+H]⁺=543.2. ¹H NMR (300 MHz, Methanol-d₄) δ 7.99 (d, J=8.1 Hz, 1H), 7.24 (d, J=3.1 Hz, 1H), 6.97 (s, 1H), 6.73 (s, 2H), 6.40 (d, J=3.1 Hz, 1H), 6.20-5.73 (m, 1H), 4.17 (d, J=12.6 Hz, 1H), 4.02-3.88 (m, 3H), 3.77 (s, 3H), 3.72-3.70 (m, 1H), 3.61-3.44 (m, 2H), 3.34 (s, 1H), 3.17-3.13 (m, 1H), 3.02-2.90 (m, 3H), 2.88-2.73 (m, 2H), 2.71-2.53 (m, 2H), 2.48 (d, J=0.8 Hz, 3H), 2.07-2.03 (m, 2H), 1.63-1.44 (m, 2H).

### Example 36. (S)-4-(4-(2,2-Difluoroethyl)-1-((5-methoxy-7-methyl-1H-indol-4-yl)methyl)piperazin-2-yl)-2-propionamidobenzoic acid & (R)-4-(4-(2,2-difluoroethyl)-1-((5-methoxy-7-methyl-1H-indol-4-yl)methyl)piperazin-2-yl)-2-propionamidobenzoic acid

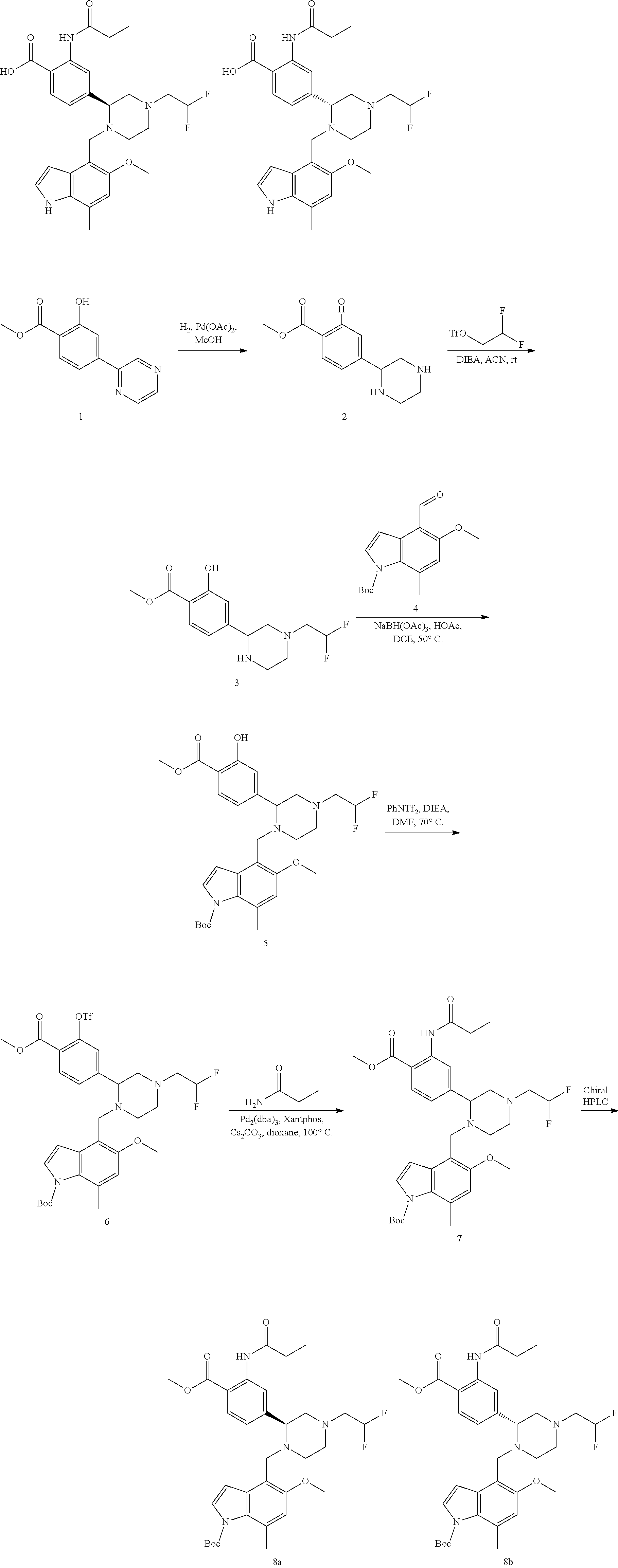

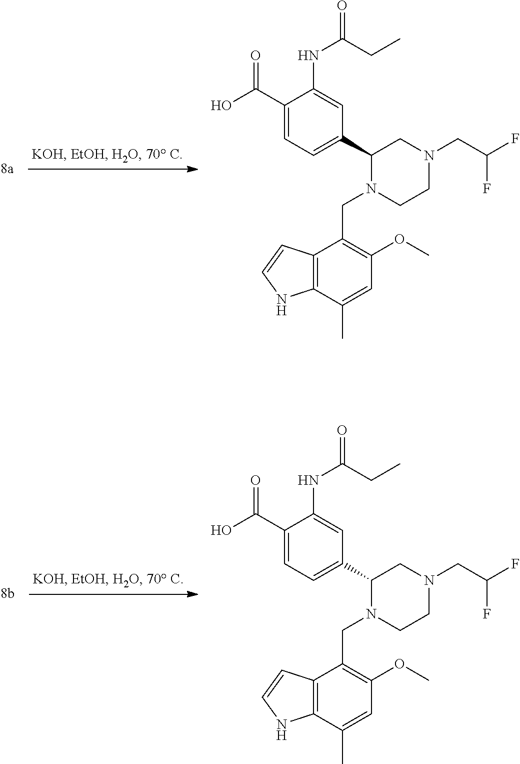 [SMILES: CCC(=O)Nc1cc([C@H]2CN(CC(F)F)CCN2Cc2c(OC)cc(C)c3[nH]ccc23)ccc1C(=O)O ; CCC(=O)Nc1cc([C@@H]2CN(CC(F)F)CCN2Cc2c(OC)cc(C)c3[nH]ccc23)ccc1C(=O)O]

Step 1: Methyl 2-hydroxy-4-(piperazin-2-yl)benzoate. Under hydrogen, a mixture of methyl 2-hydroxy-4-(pyrazin-2-yl)benzoate (7.0 g, 30.40 mmol), palladium (II) acetate (2.05 g, 9.12 mmol) in methanol (70 mL) was stirred for 12 h at room temperature. The solids were filtered out. The filtrate was concentrated under reduced pressure to afford the title compound (7.0 g, 97.4% yield) as a yellow solid. LCMS (ESI): [M+H]⁺=237.1.

Step 2: Methyl 4-(4-(2,2-difluoroethyl)piperazin-2-yl)-2-hydroxybenzoate A solution of methyl 2-hydroxy-4-(piperazin-2-yl)benzoate (7.0 g, 29.62 mmol) in acetonitrile (100 mL) was added 2,2-difluoroethyl trifluoromethanesulfonate (5.7 g, 26.66 mmol) and N,N-diisopropylethylamine (11.5 g, 88.87 mmol) at room temperature. The resulting mixture was stirred for additional 1 h. The reaction was quenched with brine and extracted with ethyl acetate. The solvent was removed under vacuum. The residue was purified by silica gel column chromatography, eluted with dichloromethane/methanol (20:1) to afford the title compound (5.3 g, 59.6% yield) as a yellow oil. LCMS (ESI): [M+H]⁺=301.1.

Step 3: tert-Butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate. A solution of methyl 4-(4-(2,2-difluoroethyl)piperazin-2-yl)-2-hydroxybenzoate (1.0 g, 3.33 mmol), tert-butyl 4-formyl-5-methoxy-7-methylindole-1-carboxylate (1.15 g, 3.99 mmol), HOAc (0.6 g, 9.99 mmol) and sodium triacetoxyborohydride (1411 mg, 6.65 mmol) in dichloroethane (10 mL) was stirred for 12 h at 50° C. The solvent was removed under vacuum. The residue was purified by reverse phase flash chromatography with MeCN/10 mmol/L NH₄HCO₃) to afford the title compound (500 mg, 26.2% yield) as a yellow oil. LCMS (ESI): [M+H]⁺=574.3.

Step 4: tert-Butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate. A solution of tert-Butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (7 g, 12.20 mmol) in DMF (100 mL) was added N-Phenyl-bis(trifluoromethanesulfonimide) (8.72 g, 24.40 mmol) and N,N-diisopropylethylamine (4.73 g, 36.60 mmol) and stirred for 5 h at 70° C. The reaction was quenched with brine and extracted with ethyl acetate. The combined organic layers were washed with water, dried over anhydrous sodium sulfate. After filtration, the filtrate was concentrated under reduced pressure. The residue was purified by silica gel column chromatography, eluted with ethyl acetate/petroleum ether (1/4) to afford the title compound (7 g, 81.3% yield) as a yellow solid. LCMS (ESI): [M+H]⁺=706.2.

Step 5: tert-Butyl 4-((4-(2,2-difluoroethyl)-2-(4-(methoxycarbonyl)-3-propionamidophenyl)piperazin-1-yl)methyl)-5-methoxy-7-methyl-1H-indole-1-carboxylate. Under nitrogen, a solution of tert-Butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (500 mg, 0.70 mmol), propionamide (155 mg, 2.12 mmol), tris(dibenzylidencacetone)dipalladium (129 mg, 0.14 mmol) and XantPhos (123 mg, 0.21 mmol) and cesium carbonate (692 mg, 2.12 mmol) in dioxane (5 mL) was stirred for 5 h at 100° C. The solids were filtered out. The filtrate was concentrated under vacuum. The residue was purified by silica gel column chromatography, eluted with ethyl acetate/petroleum ether (1/10) to afford the title compound (200 mg, 44.9% yield). LCMS (ESI): [M+H]⁺=629.3.

Step 6: tert-Butyl (S)-4-((4-(2,2-difluoroethyl)-2-(4-(methoxycarbonyl)-3-propionamidophenyl)piperazin-1-yl)methyl)-5-methoxy-7-methyl-1H-indole-1-carboxylate (isomer 1, faster peak) & tert-Butyl (R)-4-((4-(2,2-difluoroethyl)-2-(4-(methoxycarbonyl)-3-propionamidophenyl)piperazin-1-yl)methyl)-5-methoxy-7-methyl-1H-indole-1-carboxylate (isomer 2, slower peak).

tert-Butyl 4-((4-(2,2-difluoroethyl)-2-(4-(methoxycarbonyl)-3-propionamidophenyl)piperazin-1-yl)methyl)-5-methoxy-7-methyl-1H-indole-1-carboxylate (200 mg) was applied for further separation by Chiral HPLC.

tert-Butyl (S)-4-((4-(2,2-difluoroethyl)-2-(4-(methoxycarbonyl)-3-propionamidophenyl)piperazin-1-yl)methyl)-5-methoxy-7-methyl-1H-indole-1-carboxylate (80 mg 40.0% yield, isomer 1, faster peak). LCMS (ESI) [M+H]⁺=629.3. t_{R}=2.85 min ((R,R)Whelk-O1, 4.6*50 mm, 3.5 μm), Hex(0.1% DEA):IPA=80:20, 1.0 mL/min, 254 nm).

tert-Butyl (R)-4-((4-(2,2-difluoroethyl)-2-(4-(methoxycarbonyl)-3-propionamidophenyl)piperazin-1-yl)methyl)-5-methoxy-7-methyl-1H-indole-1-carboxylate (70 mg 35.0% yield, isomer 2, slower peak). LCMS (ESI) [M+H]⁺=629.3. t_{R}=3.47 min ((R,R)Whelk-O1, 4.6*50 mm, 3.5 μm), Hex(0.1% DEA):IPA=80:20, 1.0 mL/min, 254 nm).

Step 7: (S)-4-(4-(2,2-Difluoroethyl)-1-((5-methoxy-7-methyl-1H-indol-4-yl)methyl)piperazin-2-yl)-2-propionamidobenzoic acid & (R)-4-(4-(2,2-difluoroethyl)-1-((5-methoxy-7-methyl-1H-indol-4-yl)methyl)piperazin-2-yl)-2-propionamidobenzoic acid.

A solution of tert-butyl (S)-4-((4-(2,2-difluoroethyl)-2-(4-(methoxycarbonyl)-3-propionamidophenyl)piperazin-1-yl)methyl)-5-methoxy-7-methyl-1H-indole-1-carboxylate (80 mg, 0.12 mmol) in ethanol (2.5 mL) and water (0.5 mL) was added Potassium hydroxide (100 mg, 1.78 mmol) and stirred for 3 h at 70° C. The reaction mixture was acidified to pH=6 with sat. citric acid solution. The solvent was removed under vacuum. The residue was purified by reversed-phase flash chromatography with MeCN/10 mmol/L NH₄HCO₃) to afford (S)-4-(4-(2,2-Difluoroethyl)-1-((5-methoxy-7-methyl-1H-indol-4-yl)methyl)piperazin-2-yl)-2-propionamidobenzoic acid.

(S)-4-(4-(2,2-Difluoroethyl)-1-((5-methoxy-7-methyl-1H-indol-4-yl)methyl)piperazin-2-yl)-2-propionamidobenzoic acid (30.1 mg, 45.97%) as a white solid. LCMS (ESI) [M+H]⁺=515.2. ¹H NMR (300 MHz, Methanol-d₄) δ 8.80 (d, J=1.8 Hz, 1H), 8.25 (d, J=8.1 Hz, 1H), 7.37-7.28 (m, 2H), 6.75 (s, 1H), 6.35 (s, 1H), 6.22-5.78 (m, 1H), 4.52 (d, J=10.8 Hz, 1H), 4.38 (d, J=12.3 Hz, 1H), 4.16 (d, J=12.8 Hz, 1H), 3.74 (d, J=1.6 Hz, 3H), 3.40 (s, 2H), 3.19 (s, 1H), 3.12-3.08 (m, 1H), 2.96-2.85 (m, 3H), 2.76 (s, 1H), 2.55-2.51 (m, 5H), 1.30 (t, J=7.6 Hz, 3H).

(R)-4-(4-(2,2-Difluoroethyl)-1-((5-methoxy-7-methyl-1H-indol-4-yl)methyl)piperazin-2-yl)-2-propionamidobenzoic acid was prepared according to above.

(R)-4-(4-(2,2-Difluoroethyl)-1-((5-methoxy-7-methyl-1H-indol-4-yl)methyl)piperazin-2-yl)-2-propionamidobenzoic acid (29.3 mg, 51.1% yield) as a white solid. LCMS (ESI) [M+H]⁺=515.2. ¹H NMR (300 MHz, Methanol-d₄) δ 8.80 (d, J=1.8 Hz, 1H), 8.25 (d, J=8.1 Hz, 1H), 7.37-7.28 (m, 2H), 6.75 (s, 1H), 6.35 (s, 1H), 6.22-5.78 (m, 1H), 4.52 (d, J=10.8 Hz, 1H), 4.38 (d, J=12.3 Hz, 1H), 4.16 (d, J=12.8 Hz, 1H), 3.74 (d, J=1.6 Hz, 3H), 3.40 (s, 2H), 3.19 (s, 1H), 3.12-3.08 (m, 1H), 2.96-2.85 (m, 3H), 2.76 (s, 1H), 2.55-2.51 (m, 5H), 1.30 (t, J=7.6 Hz, 3H).

### Example 37. (S)-4-(1-((5-Methoxy-7-methyl-1H-indol-4-yl)methyl)-4-(3,3,3-trifluoropropyl)piperazin-2-yl)-2-(2-oxopyrrolidin-1-yl)benzamide & (R)-4-(1-((5-methoxy-7-methyl-1H-indol-4-yl)methyl)-4-(3,3,3-trifluoropropyl)piperazin-2-yl)-2-(2-oxopyrrolidin-1-yl)benzamide

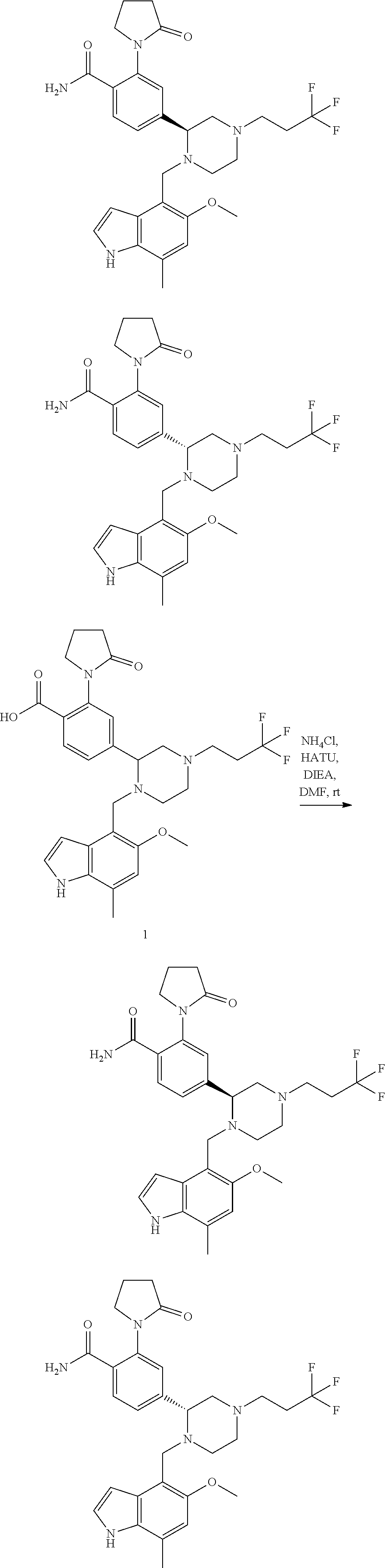

To a stirred mixture of 4-(1-((5-methoxy-7-methyl-1H-indol-4-yl)methyl)-4-(3,3,3-trifluoropropyl)piperazin-2-yl)-2-(2-oxopyrrolidin-1-yl)benzoic acid (70 mg, 0.13 mmol), NH₄Cl (70 mg, 1.31 mmol) and DIEA (55 mg, 0.43 mmol) in DMF (2.5 mL) was added HATU (70 mg, 0.18 mmol). The resulting mixture was stirred for 1 h at room temperature. The residue was purified by reverse phase flash with the following conditions (acetonitrile, 5-50%/10 mM NH₄HCO₃ in water) to afford the title compound as racemate. The racemate was applied for further purification by Chiral HPLC with the folloing condition: Column: CHIRAL ART Amylose-SA, 2*25 cm, 5 μm; Mobile Phase A: Hex(0.5% of 2 M NH₃-MeOH)-HPLC, Mobile Phase B: Ethanol-HPLC; Flow rate: 20 mL/min; Gradient: 30% B to 30% B in 14 min; Wave Length: 220/254 nm; RT1(min): 9.261; RT2(min): 11.629; Sample Solvent: Ethanol-HPLC; Injection Volume: 0.5 mL; Number of Runs: 5.

(S)-4-(1-((5-Methoxy-7-methyl-1H-indol-4-yl)methyl)-4-(3,3,3-trifluoropropyl)piperazin-2-yl)-2-(2-oxopyrrolidin-1-yl)benzamide (isomer 1, faster peak) (17.9 mg, 25.54% yield) as a white solid. LCMS (ESI): [M+H]⁺=558.2. t_{R}=1.61 min (CHIRALPAK IA-3, 4.6*50 mm, 3 μm, Hex(0.1% DEA):Ethanol=70:30, 1.0 mL/min, 254 nm). ¹H NMR (300 MHz, DMSO-d₆) δ 10.84 (s, 1H), 7.67 (s, 1H), 7.61-7.41 (m, 3H), 7.25 (t, J=2.8 Hz, 1H), 7.20 (s, 1H), 6.66 (s, 1H), 6.51-6.36 (m, 1H), 3.81 (t, J=7.4 Hz, 2H), 3.71 (s, 3H), 3.63 (d, J=11.8 Hz, 1H), 3.39-3.35 (m, 1H), 3.23 (d, J=11.8 Hz, 1H), 2.76 (t, J=12.2 Hz, 2H), 2.62 (d, J=11.3 Hz, 1H), 2.48-2.39 (m, 7H), 2.34 (t, J=7.9 Hz, 2H), 2.29-1.96 (m, 5H).

(R)-4-(1-((5-Methoxy-7-methyl-1H-indol-4-yl)methyl)-4-(3,3,3-trifluoropropyl)piperazin-2-yl)-2-(2-oxopyrrolidin-1-yl)benzamide (isomer 2, slower peak) (18.2 mg, 25.99% yield) as a white solid. LCMS (ESI): [M+H]⁺=558.2. t_{R}=2.37 min (CHIRALPAK IA-3, 4.6*50 mm, 3 μm, Hex(0.1% DEA):Ethanol=70:30, 1.0 mL/min, 254 nm), ¹H NMR (300 MHz, DMSO-d₆) δ 10.84 (s, 1H), 7.68 (s, 1H), 7.63-7.41 (m, 3H), 7.25 (t, J=2.8 Hz, 1H), 7.21 (s, 1H), 6.67 (s, 1H), 6.50-6.39 (m, 1H), 3.82 (t, J=7.5 Hz, 2H), 3.72 (s, 3H), 3.64 (d, J=11.8 Hz, 1H), 3.41-3.36 (m, 1H), 3.24 (d, J=11.8 Hz, 1H), 2.76 (t, J=12.2 Hz, 2H), 2.63 (d, J=11.3 Hz, 1H), 2.49-2.40 (m, 7H), 2.35 (t, J=7.9 Hz, 2H), 2.29-1.89 (m, 5H).

### Example 38. 2-Amino-6-((2R)-1-((5-methoxy-7-methyl-1H-indol-4-yl)methyl)-4-(3,3,3-trifluoropropyl)piperazin-2-yl)pyridine-3-carboxamide & 2-amino-6-((2S)-1-((5-methoxy-7-methyl-1H-indol-4-yl)methyl)-4-(3,3,3-trifluoropropyl)piperazin-2-yl)pyridine-3-carboxamide

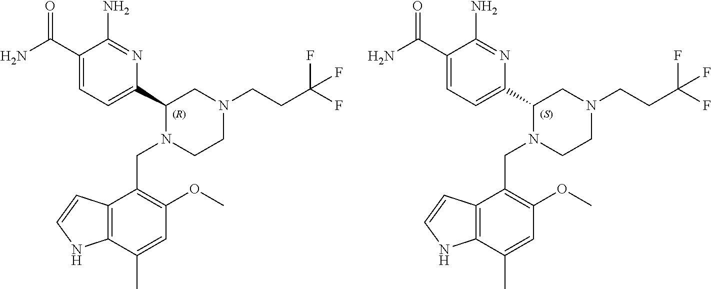

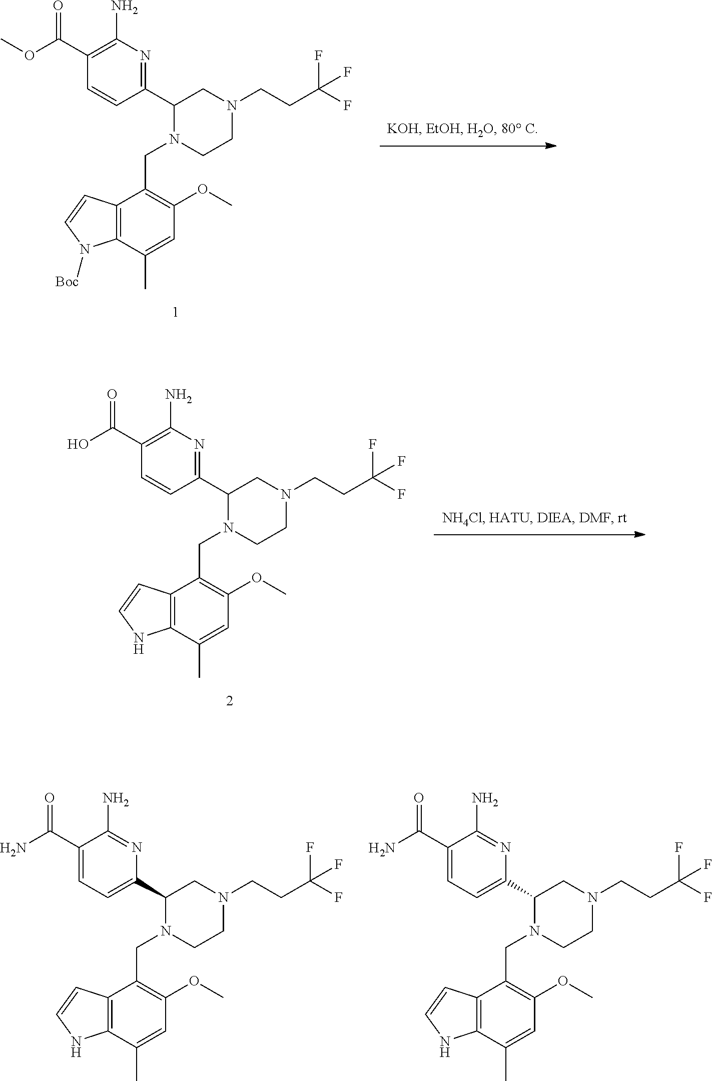 [SMILES: COC(=O)c1ccc(C2CN(CCC(F)(F)F)CCN2Cc2c(OC)cc(C)c3c2ccn3C(=O)OC(C)(C)C)nc1N ; COc1cc(C)c2[nH]ccc2c1CN1CCN(CCC(F)(F)F)CC1c1ccc(C(=O)O)c(N)n1 ; COc1cc(C)c2[nH]ccc2c1CN1CCN(CCC(F)(F)F)C[C@@H]1c1ccc(C(N)=O)c(N)n1 ; COc1cc(C)c2[nH]ccc2c1CN1CCN(CCC(F)(F)F)C[C@H]1c1ccc(C(N)=O)c(N)n1]

Step 1: 2-Amino-6-(1-((5-methoxy-7-methyl-1H-indol-4-yl)methyl)-4-(3,3,3-trifluoropropyl)piperazin-2-yl) nicotinic acid. To a mixture of tert-butyl 4-((2-(6-amino-5-(methoxycarbonyl)pyridin-2-yl)-4-(3,3,3-trifluoropropyl)piperazin-1-yl)methyl)-5-methoxy-7-methylindole-1-carboxylate (180 mg, 0.30 mmol) and Potassium hydroxide (167 mg, 2.98 mmol) in water (1.5 mL) and Ethanol (3 mL) was stirred for 1 h at 80° C. The mixture was acidified to pH=6 with sat. citric acid. The residue was purified by reverse phase flash with the following conditions (acetonitrile, 5-30%/10 mM NH₄HCO₃ in water) to afford the title compound (101 mg, 69.14% yield) as a light yellow solid. LCMS (ESI): [M+H]⁺=492.2.

Step 2: 2-amino-6-((2R)-1-((5-methoxy-7-methyl-1H-indol-4-yl)methyl)-4-(3,3,3-trifluoropropyl)piperazin-2-yl)pyridine-3-carboxamide & 2-amino-6-((2S)-1-((5-methoxy-7-methyl-1H-indol-4-yl)methyl)-4-(3,3,3-trifluoropropyl)piperazin-2-yl)pyridine-3-carboxamide.

To a stirred mixture of 2-amino-6-(1-((5-methoxy-7-methyl-1H-indol-4-yl)methyl)-4-(3,3,3-trifluoropropyl)piperazin-2-yl)pyridine-3-carboxylic acid (100 mg, 0.20 mmol), NH₄Cl (110 mg, 2.06 mmol) and DIEA (150 mg, 1.16 mmol) in DMF (3 mL) was added HATU (120 mg, 0.32 mmol). The resulting mixture was stirred for 1 h at room temperature. The residue was purified by reverse phase flash with the following conditions (acetonitrile, 20-50%/10 mM NH₄HCO₃ in water) to afford the title compound (90 mg, 90.18% yield) as a light yellow solid and as racemate. The racemate was applied for further separation by Chiral HPLC with the folloing condition: Column: CHIRAL ART Amylose-SA, 2*25 cm, 5 μm; Mobile Phase A: Hex (0.5% 2M of NH₃-MeOH)-HPLC, Mobile Phase B: Ethanol-HPLC; Flow rate: 20 mL/min; Gradient: 30% B to 30% B in 19 min; Wave Length: 220/254 nm; RT1(min): 9.537; RT2(min): 14.835; Sample Solvent: Ethanol-HPLC; Injection Volume: 1.2 mL; Number Of Runs: 3.

2-Amino-6-((2R)-1-((5-methoxy-7-methyl-1H-indol-4-yl)methyl)-4-(3,3,3-trifluoropropyl)piperazin-2-yl)pyridine-3-carboxamide (isomer 1, faster peak) (28.9 mg, 28.93% yield) as a white solid. LCMS (ESI): [M+H]⁺=491.2. t_{R}=1.78 min (CHIRALPAK IE-3, 4.6*50 mm, 3 μm, Hex(0.1% DEA):Ethanol=75:25, 1.0 mL/min, 254 nm), ¹H NMR (300 MHz, DMSO-d₆) δ 10.84 (s, 1H), 8.03 (d, J=8.0 Hz, 1H), 7.91 (s, 1H), 7.39-7.08 (m, 4H), 6.96 (d, J=7.9 Hz, 1H), 6.67 (s, 1H), 6.61-6.50 (m, 1H), 3.72 (s, 3H), 3.66 (d, J=11.9 Hz, 1H), 3.31-3.12 (m, 2H), 2.83 (d, J=10.6 Hz, 1H), 2.71 (d, J=10.6 Hz, 1H), 2.61 (d, J=11.4 Hz, 1H), 2.48-2.36 (m, 7H), 2.25-1.85 (m, 3H).

2-Amino-6-((2S)-1-((5-methoxy-7-methyl-1H-indol-4-yl)methyl)-4-(3,3,3-trifluoropropyl)piperazin-2-yl)pyridine-3-carboxamide (isomer 2, slower peak) (29.7 mg, 29.70% yield) as a white solid. LCMS (ESI): [M+H]⁺=491.2. t_{R}=2.90 min (CHIRALPAK IE-3 (4.6*50 mm, 3 μm), Hex(0.1% DEA):Ethanol=75:25, 1.0 mL/min, 254 nm), ¹H NMR (300 MHz, DMSO-d₆) δ 10.84 (s, 1H), 8.03 (d, J=8.0 Hz, 1H), 7.91 (s, 1H), 7.45-7.07 (m, 4H), 6.96 (d, J=7.9 Hz, 1H), 6.67 (s, 1H), 6.61-6.49 (m, 1H), 3.73 (s, 3H), 3.66 (d, J=11.8 Hz, 1H), 3.32-3.14 (m, 2H), 2.83 (d, J=10.6 Hz, 1H), 2.71 (d, J=10.5 Hz, 1H), 2.61 (d, J=11.4 Hz, 1H), 2.48-2.26 (m, 7H), 2.25-1.84 (m, 3H).

### Example 39. 2-Amino-4-((2S)-1-((5-methoxy-7-methyl-1H-indol-4-yl)methyl)-4-(3,3,3-trifluoropropyl)piperazin-2-yl)benzamide & 2-amino-4-((2R)-1-((5-methoxy-7-methyl-1H-indol-4-yl)methyl)-4-(3,3,3-trifluoropropyl)piperazin-2-yl)benzamide

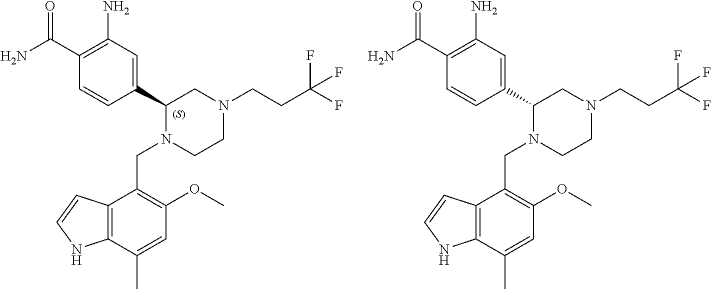

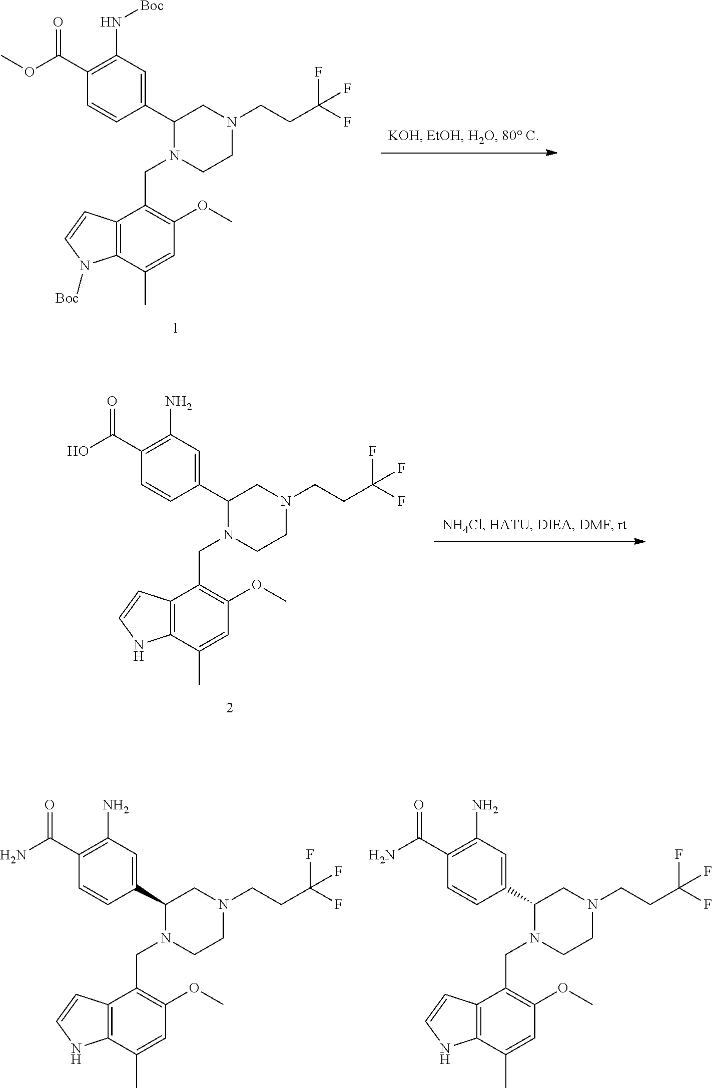 [SMILES: COC(=O)c1ccc(C2CN(CCC(F)(F)F)CCN2Cc2c(OC)cc(C)c3c2ccn3C(=O)OC(C)(C)C)cc1NC(=O)OC(C)(C)C ; COc1cc(C)c2[nH]ccc2c1CN1CCN(CCC(F)(F)F)CC1c1ccc(C(=O)O)c(N)c1 ; COc1cc(C)c2[nH]ccc2c1CN1CCN(CCC(F)(F)F)C[C@@H]1c1ccc(C(N)=O)c(N)c1 ; COc1cc(C)c2[nH]ccc2c1CN1CCN(CCC(F)(F)F)C[C@H]1c1ccc(C(N)=O)c(N)c1]

Step 1: 2-Amino-4-(1-((5-methoxy-7-methyl-1H-indol-4-yl)methyl)-4-(3,3,3-trifluoropropyl)piperazin-2-yl)benzoic acid. To a mixture of tert-butyl 4-((2-(3-((tert-butoxycarbonyl)amino)-4-(methoxycarbonyl)phenyl)-4-(3,3,3-trifluoropropyl)piperazin-1-yl)methyl)-5-methoxy-7-methylindole-1-carboxylate (100 mg, 0.14 mmol) and Potassium hydroxide (80 mg, 1.43 mmol) in Ethanol (3 mL) and water (1.5 mL) was stirred for 5 h at 100° C. The mixture was allowed to cool down to room temperature. The mixture was acidified to pH 6 with citric acid. The residue was purified by reverse phase flash with the following conditions (acetonitrile, 5-30%/10 mM NH₄HCO₃ in water) to afford the title compound (55 mg, 79.02% yield) as a light yellow solid. LCMS (ESI): [M+H]⁺=491.2.

Step 2: 2-Amino-4-((2S)-1-((5-methoxy-7-methyl-1H-indol-4-yl)methyl)-4-(3,3,3-trifluoropropyl)piperazin-2-yl)benzamide & 2-amino-4-((2R)-1-((5-methoxy-7-methyl-1H-indol-4-yl)methyl)-4-(3,3,3-trifluoropropyl)piperazin-2-yl)benzamide. To a mixture of 2-amino-4-(1-((5-methoxy-7-methyl-1H-indol-4-yl)methyl)-4-(3,3,3-trifluoropropyl)piperazin-2-yl)benzoic acid (50 mg, 0.10 mmol), NH₄Cl (60 mg, 1.12 mmol), DIEA (40 mg, 0.31 mmol) and HATU (60 mg, 0.16 mmol) in DMF (2 mL) was stirred for 2 h at room temperature. The residue was purified by reverse phase flash with the following conditions (acetonitrile, 5-50%/10 mM NH₄HCO₃ in water) and further applied for further separation by Chiral HPLC with the folloing condition: Column: CHIRAL ART Amylose-SA, 2*25 cm, 5 μm; Mobile Phase A: Hex(0.5% of 2 M NH₃-MeOH)-HPLC, Mobile Phase B: Ethanol-HPLC; Flow rate: 20 mL/min; Gradient: 30% B to 30% B in 19 min; Wave Length: 220/254 nm; RT1(min): 9.537; RT2(min): 14.835; Sample Solvent: Ethanol-HPLC; Injection Volume: 1.2 mL; Number of Runs: 3.

2-amino-4-((2S)-1-((5-methoxy-7-methyl-1H-indol-4-yl)methyl)-4-(3,3,3-trifluoropropyl)piperazin-2-yl)benzamide (isomer 1, faster peak) (16.5 mg, 30.06% yield) as a white solid. LCMS (ESI): [M+H]⁺=490.4. t_{R}=1.99 min (CHIRALPAK IA-3, 4.6*50 mm, 3 um, Hex(0.1% DEA):Ethanol=70:30, 1.0 mL/min, 254 nm). ¹H NMR (300 MHz, Methanol-d₄) δ 7.58 (d, J=8.2 Hz, 1H), 7.19 (d, J=3.1 Hz, 1H), 6.95 (s, 1H), 6.85 (d, J=8.2 Hz, 1H), 6.68 (s, 1H), 6.45 (d, J=3.1 Hz, 1H), 3.86 (d, J=11.8 Hz, 1H), 3.78 (s, 3H), 3.38-3.34 (m, 1H), 3.26 (s, 1H), 2.93-2.73 (m, 3H), 2.67-2.52 (m, 2H), 2.46 (s, 3H), 2.43-2.30 (m, 3H), 2.30-2.10 (m, 2H).

2-amino-4-((2R)-1-((5-methoxy-7-methyl-1H-indol-4-yl)methyl)-4-(3,3,3-trifluoropropyl)piperazin-2-yl)benzamide (isomer 2, slower peak) (18.4 mg, 33.52% yield) as a white solid. LCMS (ESI): [M+H]⁺=490.1. t_{R}=2.61 min (CHIRALPAK IA-3, 4.6*50 mm, 3 μm, Hex(0.1% DEA):Ethanol=70:30, 1.0 mL/min, 254 nm). ¹H NMR (300 MHz, Methanol-d₄) δ 7.60 (d, J=8.2 Hz, 1H), 7.21 (d, J=3.1 Hz, 1H), 6.97 (s, 1H), 6.88 (d, J=8.2 Hz, 1H), 6.71 (s, 1H), 6.47 (d, J=3.1 Hz, 1H), 3.88 (d, J=11.8 Hz, 1H), 3.80 (s, 3H), 3.38-3.35 (m, 1H), 3.29 (s, 1H), 2.95-2.77 (m, 3H), 2.70-2.54 (m, 2H), 2.48 (s, 3H), 2.46-2.33 (m, 3H), 2.33-2.14 (m, 2H).

### Example 40. 2-Amino-6-((2R)-1-((5-methoxy-7-methyl-1H-indol-4-yl)methyl)-4-(3,3,3-trifluoropropyl)piperazin-2-yl)pyridine-3-carboxylic acid & 2-amino-6-((2S)-1-((5-methoxy-7-methyl-1H-indol-4-yl)methyl)-4-(3,3,3-trifluoropropyl)piperazin-2-yl)pyridine-3-carboxylic acid

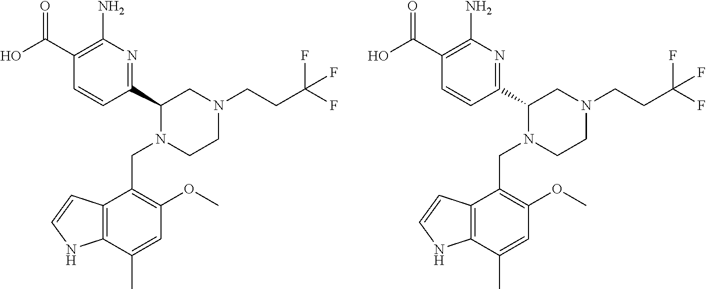 [SMILES: COc1cc(C)c2[nH]ccc2c1CN1CCN(CCC(F)(F)F)C[C@@H]1c1ccc(C(=O)O)c(N)n1 ; COc1cc(C)c2[nH]ccc2c1CN1CCN(CCC(F)(F)F)C[C@H]1c1ccc(C(=O)O)c(N)n1]

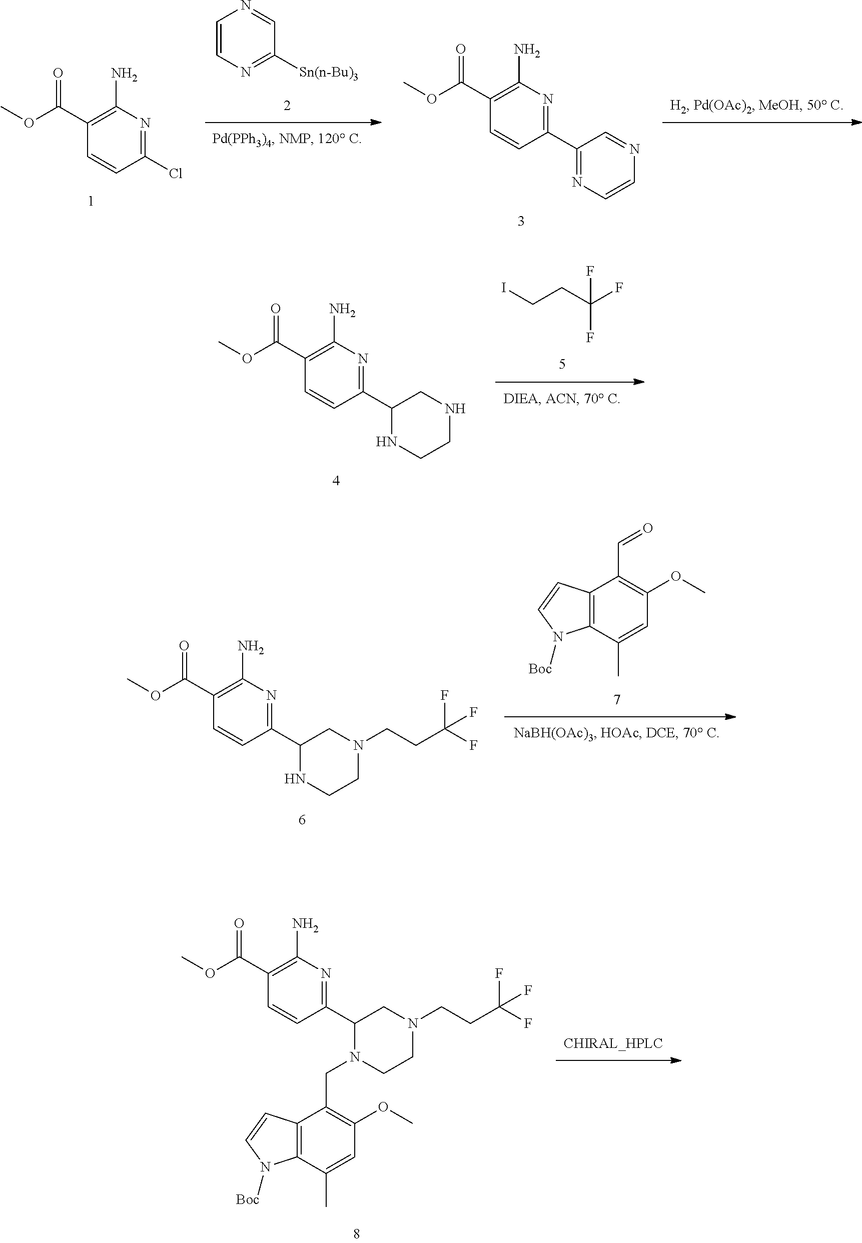

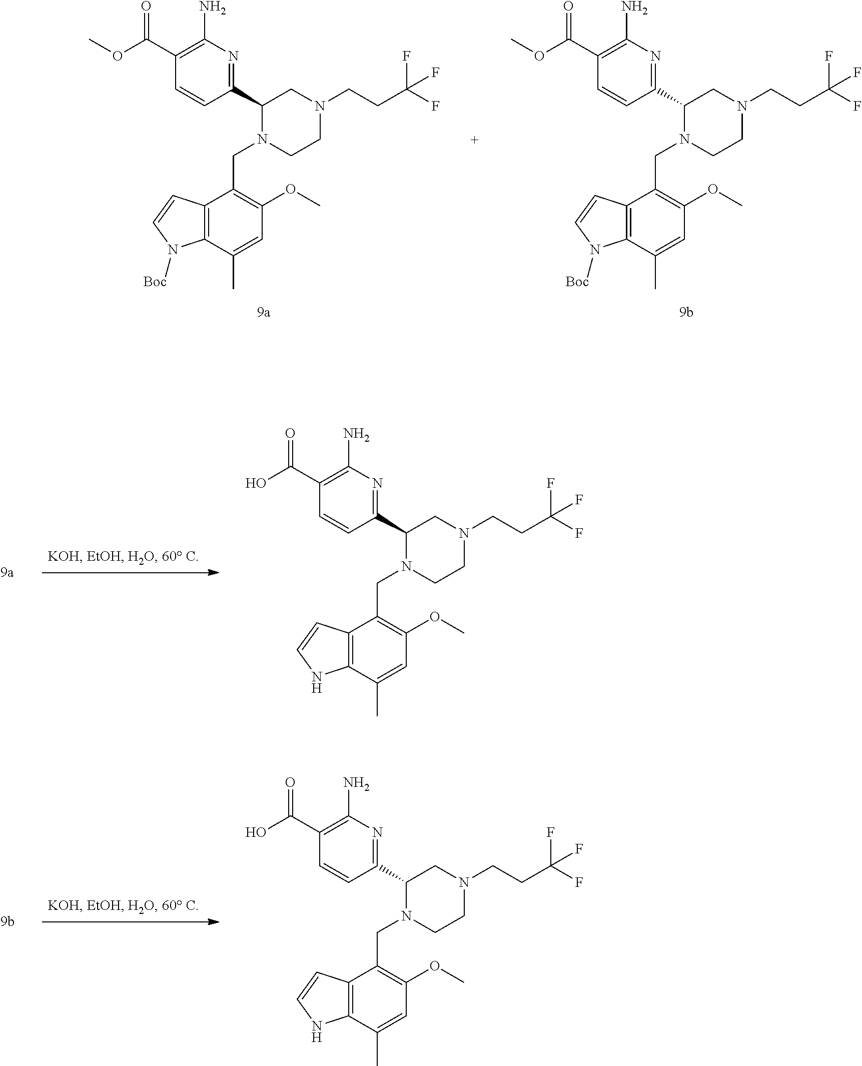 [SMILES: COC(=O)c1ccc([C@H]2CN(CCC(F)(F)F)CCN2Cc2c(OC)cc(C)c3c2ccn3C(=O)OC(C)(C)C)nc1N ; COC(=O)c1ccc([C@@H]2CN(CCC(F)(F)F)CCN2Cc2c(OC)cc(C)c3c2ccn3C(=O)OC(C)(C)C)nc1N ; COc1cc(C)c2[nH]ccc2c1CN1CCN(CCC(F)(F)F)C[C@@H]1c1ccc(C(=O)O)c(N)n1 ; COc1cc(C)c2[nH]ccc2c1CN1CCN(CCC(F)(F)F)C[C@H]1c1ccc(C(=O)O)c(N)n1]

Step 1: Methyl 2-amino-6-(pyrazin-2-yl)pyridine-3-carboxylate. To a mixture of methyl 2-amino-6-chloropyridine-3-carboxylate (1.5 g, 8.04 mmol) and 2-(tributylstannyl)pyrazine (3.0 g, 8.12 mmol) in NMP (20 mL) were added Pd(PPh₃)₄ (930 mg, 0.81 mmol). The resulting mixture was stirred for overnight at 120° C. under nitrogen atmosphere. The reaction was quenched with sat. KF (aq.) at room temperature. The resulting mixture was extracted with ethyl acetate. The combined organic layers were washed with brine, dried over anhydrous Na₂SO₄. After filtration, the filtrate was concentrated under reduced pressure. The residue was purified by silica gel column chromatography, eluted with petroleum ether/ethyl acetate (3:2) to afford the title compound (1.3 g, 70.24% yield) as a yellow solid. LCMS (ESI): [M+H]⁺=231.2.

Step 2: Methyl 2-amino-6-(piperazin-2-yl)pyridine-3-carboxylate. To a mixture of methyl 2-amino-6-(pyrazin-2-yl)pyridine-3-carboxylate (830 mg, 3.61 mmol) and Pd(OAc)₂ (162 mg, 0.72 mmol) in MeOH (30 mL) was stirred for overnight at 50° C. under hydrogen atmosphere. The resulting mixture was filtered, the filter cake was washed with MeOH. The filtrate was concentrated under reduced pressure to afford the title compound (830 mg, 97.22% yield) as a brown oil. LCMS (ESI): [M+H]⁺=237.1.

Step 3: Methyl 2-amino-6-(4-(3,3,3-trifluoropropyl)piperazin-2-yl)pyridine-3-carboxylate. To a mixture of methyl 2-amino-6-(piperazin-2-yl)pyridine-3-carboxylate (830 mg, 3.52 mmol), N,N-diisopropylethylamine (1.8 g, 13.97 mmol) and 1,1,1-trifluoro-3-iodopropane (1.2 g, 5.12 mmol) in acetonitrile (10 mL) was stirred for 4 h at 80° C. The residue was purified by reverse phase flash with the following conditions (acetonitrile, 10-50%/10 mM NH₄HCO₃ in water) to afford the title compound (438 mg, 28.31% yield) as a brown solid. LCMS (ESI): [M+H]⁺=333.3.

Step 4: tert-Butyl 4-((2-(6-amino-5-(methoxycarbonyl)pyridin-2-yl)-4-(3,3,3-trifluoropropyl)piperazin-1-yl)methyl)-5-methoxy-7-methylindole-1-carboxylate. To a mixture of methyl 2-amino-6-(4-(3,3,3-trifluoropropyl)piperazin-2-yl)pyridine-3-carboxylate (420 mg, 1.26 mmol), tert-butyl 4-formyl-5-methoxy-7-methylindole-1-carboxylate (548 mg, 1.90 mmol), HOAc (152 mg, 2.53 mmol) and NaBH(OAc)₃ (804 mg, 3.79 mmol) in 1,2-dichloroethane (4 mL) was stirred for 4 h at 70° C. The resulting mixture was diluted with ethyl acetate. The residue was washed with water. The organic layer was concentrated under reduced pressure. The residue was purified by silica gel column chromatography, eluted with petroleum ether/ethyl acetate (5:1) to afford the title compound (340 mg, 44.42% yield) as a brown oil. LCMS (ESI): [M+H]⁺=606.2.

Step 5: tert-butyl 4-(((2R)-2-(6-amino-5-(methoxycarbonyl)pyridin-2-yl)-4-(3,3,3-trifluoropropyl)piperazin-1-yl)methyl)-5-methoxy-7-methylindole-1-carboxylate (isomer 1, faster peak) & tert-butyl 4-(((2S)-2-(6-amino-5-(methoxycarbonyl)pyridin-2-yl)-4-(3,3,3-trifluoropropyl)piperazin-1-yl)methyl)-5-methoxy-7-methylindole-1-carboxylate (isomer 2, slower peak).

The tert-butyl 4-((2-(6-amino-5-(methoxycarbonyl)pyridin-2-yl)-4-(3,3,3-trifluoropropyl)piperazin-1-yl)methyl)-5-methoxy-7-methylindole-1-carboxylate (150 mg) was applied for further separation by Chiral HPLC with the folloing condition: Column: CHIRAL ART Amylose-SA, 2*25 cm, 5 μm; Mobile Phase A: Hex(0.5% of 2M NH₃-MeOH)-HPLC, Mobile Phase B: IPA-HPLC; Flow rate: 20 mL/min; Gradient: 20% B to 20% B in 10 min; Wave Length: 220/254 nm; RT1(min): 5.918; RT2(min): 8.096; Sample Solvent: Ethanol-HPLC; Injection Volume: 0.3 mL; Number of Runs: 15.

tert-Butyl 4-(((2R)-2-(6-amino-5-(methoxycarbonyl)pyridin-2-yl)-4-(3,3,3-trifluoropropyl)piperazin-1-yl)methyl)-5-methoxy-7-methylindole-1-carboxylate (isomer 1, faster peak) (85 mg, 56.67% yield) as a light yellow solid. LCMS (ESI): [M+H]⁺=606.2. t_{R}=1.07 min, (CHIRALPAK IA-3, 4.6*50 mm, 3 μm, Hex(0.1% DEA):IPA=80:20, 1.0 mL/min, 254 nm).

tert-Butyl 4-(((2S)-2-(6-amino-5-(methoxycarbonyl)pyridin-2-yl)-4-(3,3,3-trifluoropropyl)piperazin-1-yl)methyl)-5-methoxy-7-methylindole-1-carboxylate (isomer 2, slower peak) (53 mg, 35.33% yield) as a light yellow solid. LCMS (ESI): [M+H]⁺=606.2. t_{R}=1.53 min, (CHIRALPAK IA-3, 4.6*50 mm, 3 μm, Hex(0.1% DEA):IPA-80:20, 1.0 mL/min, 254 nm).

Step 6: 2-Amino-6-((2R)-1-((5-methoxy-7-methyl-1H-indol-4-yl)methyl)-4-(3,3,3-trifluoropropyl)piperazin-2-yl)pyridine-3-carboxylic acid & 2-amino-6-((2S)-1-((5-methoxy-7-methyl-1H-indol-4-yl)methyl)-4-(3,3,3-trifluoropropyl)piperazin-2-yl)pyridine-3-carboxylic acid.

To a mixture of tert-butyl 4-(((2R)-2-(6-amino-5-(methoxycarbonyl)pyridin-2-yl)-4-(3,3,3-trifluoropropyl)piperazin-1-yl)methyl)-5-methoxy-7-methylindole-1-carboxylate (85 mg, 0.14 mmol) and Potassium hydroxide (80 mg, 1.43 mmol) in water (1.5 mL) and Ethanol (3 mL) was stirred for 1 h at 60° C. The mixture was acidified to pH=6 with citric acid. The residue was purified by reverse phase flash with the following conditions (acetonitrile, 5-30%/10 mM NH₄HCO₃ in water) to afford 2-amino-6-((2R)-1-((5-methoxy-7-methyl-1H-indol-4-yl)methyl)-4-(3,3,3-trifluoropropyl)piperazin-2-yl)pyridine-3-carboxylic acid (32.7 mg, 46.60% yield) as a light yellow solid. LCMS (ESI): [M+H]⁺=492.1; ¹H NMR (300 MHz, DMSO-d₆) δ 12.82 (s, 1H), 10.83 (s, 1H), 8.11 (d, J=8.0 Hz, 1H), 7.25 (t, J=2.7 Hz, 2H), 7.02 (d, J=8.0 Hz, 1H), 6.67 (s, 1H), 6.60-6.44 (m, 1H), 3.72 (s, 3H), 3.65 (d, J=11.8 Hz, 1H), 3.31-3.25 (m, 3H), 2.84 (d, J=10.7 Hz, 1H), 2.71 (d, J=10.6 Hz, 1H), 2.62 (d, J=11.3 Hz, 1H), 2.49-2.44 (m, 2H), 2.43 (s, 3H), 2.40-2.30 (m, 1H), 2.26-2.12 (m, 1H), 2.07 (t, J=10.6 Hz, 1H), 2.02-1.87 (m, 1H).

To a mixture of tert-butyl 4-(((2S)-2-(6-amino-5-(methoxycarbonyl)pyridin-2-yl)-4-(3,3,3-trifluoropropyl)piperazin-1-yl)methyl)-5-methoxy-7-methylindole-1-carboxylate (53 mg, 0.09 mmol) and Potassium hydroxide (50 mg, 0.89 mmol) in Ethanol (3 mL) and water (1.5 mL) was stirred for 1 h at 60° C. The mixture was acidified to pH=6 with citric acid. The residue was purified by reverse phase flash with the following conditions (acetonitrile, 5-30%/10 mM NH₄HCO₃ in water) to afford 2-amino-6-((2S)-1-((5-methoxy-7-methyl-1H-indol-4-yl)methyl)-4-(3,3,3-trifluoropropyl)piperazin-2-yl)pyridine-3-carboxylic acid (25.4 mg, 58.94% yield) as a white solid. LCMS (ESI): [M+H]⁺=492.2; ¹H NMR (300 MHz, DMSO-d₆) δ 10.83 (s, 1H), 8.11 (d, J=8.0 Hz, 1H), 7.26 (t, J=2.8 Hz, 2H), 7.02 (d, J=8.0 Hz, 1H), 6.67 (s, 1H), 6.59-6.48 (m, 1H), 3.72 (s, 3H), 3.65 (d, J=11.8 Hz, 1H), 3.31-3.17 (m, 3H), 2.84 (d, J=10.7 Hz, 1H), 2.71 (d, J=10.9 Hz, 1H), 2.62 (d, J=11.4 Hz, 1H), 2.49-2.45 (m, 2H), 2.43 (s, 3H), 2.37 (s, 1H), 2.25-2.12 (m, 1H), 2.07 (t, J=10.6 Hz, 1H), 2.01-1.86 (m, 1H).

### Example 41. 4-((3R,8aS)-7,7-Difluoro-2-((5-methoxy-7-methyl-1H-indol-4-yl)methyl)octahydropyrrolo[1,2-a]pyrazin-3-yl)-2-hydroxybenzoic acid

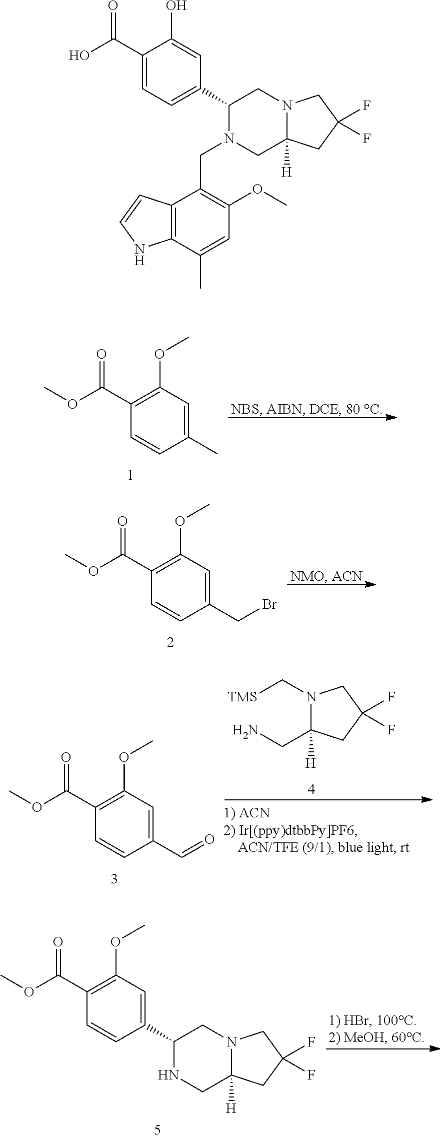

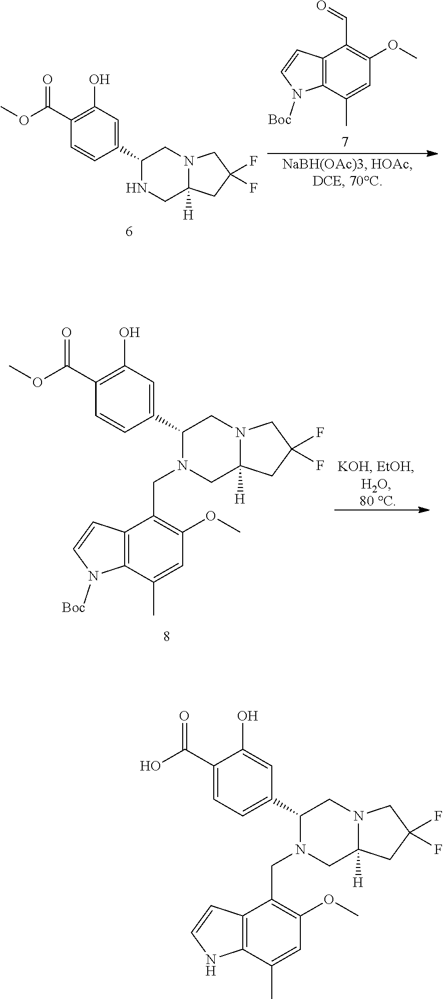 [SMILES: COC(=O)c1ccc([C@@H]2CN3CC(F)(F)C[C@H]3CN2)cc1O ; COc1cc(C)c2c(ccn2C(=O)OC(C)(C)C)c1C=O ; COC(=O)c1ccc([C@@H]2CN3CC(F)(F)C[C@H]3CN2Cc2c(OC)cc(C)c3c2ccn3C(=O)OC(C)(C)C)cc1O ; COc1cc(C)c2[nH]ccc2c1CN1C[C@@H]2CC(F)(F)CN2C[C@H]1c1ccc(C(=O)O)c(O)c1]

Step 1: Methyl 4-(bromomethyl)-2-methoxybenzoate. A mixture of methyl 2-methoxy-4-methylbenzoate (10 g, 55.49 mmol), NBS (9.88 g, 55.493 mmol) and AIBN (4.56 g, 27.747 mmol) in DCE (300 mL) was stirred for 4 h at 80° C. under nitrogen. The resulting mixture was quenched with water and extracted with ethyl acetate for 3 times. The solvent was concentrated under reduced pressure. The residue was purified by silica gel column chromatography, eluted with petroleum ether/ethyl acetate (5:1) to afford the title compound (8.4 g, 58.4% yield) as a colorless oil. LCMS (ESI): [M+H]⁺=259.0.

Step 2: Methyl 4-formyl-2-methoxybenzoate. A mixture of methyl 4-(bromomethyl)-2-methoxybenzoate (8.4 g, 32.42 mmol) and NMO (7.6 g, 64.84 mmol) in ACN (100 mL) was stirred for overnight at room temperature. The reaction was diluted with ethyl acatate, and washed with water for 3 times. The solvent was concentrated under reduced pressure. The residue was purified by silica gel column chromatography, eluted with dichloromethane/petroleum ether (1:1) to afford the title compound (1.6 g, 25.4% yield) as a colorless oil. LCMS (ESI): [M+H]⁺=195.1.

Step 3: Methyl 4-((3R,8aS)-7,7-difluorooctahydropyrrolo[1,2-a]pyrazin-3-yl)-2-methoxybenzoate. A mixture of 1-((2S)-4,4-difluoro-1-((trimethylsilyl)methyl)pyrrolidin-2-yl)methanamine (600 mg, 2.70 mmol) and methyl 4-formyl-2-methoxybenzoate (524 mg, 2.70 mmol) in ACN (12 mL) was stirred for overnight at rt. Then (Ir(dtbbpy)(ppy)₂)(PF₆) (247 mg, 0.27 mmol) and Difluoroethanol (1.3 mL) was added and stirred for 4 h at rt under nitrogen under blue LED. The solvent was removed under vacuum. The residue was purified by reverse flash chromatography with the following conditions: column, C18 silica gel; mobile phase, MeCN in Water (10 mmol/L NH₄HCO₃), 10% to 40% gradient in 30 min; detector, UV 254 nm. The solvent was concentrated under reduced pressure to afford the title compound (210 mg, 23.9% yield) as a yellow oil. LCMS (ESI): [M+H]⁺=327.1.

Step 4: Methyl 4-((3R,8aS)-7,7-difluorooctahydropyrrolo[1,2-a]pyrazin-3-yl)-2-hydroxybenzoate. A mixture of methyl 4-((3R,8aS)-7,7-difluorooctahydropyrrolo[1,2-a]pyrazin-3-yl)-2-methoxybenzoate (190 mg, 0.58 mmol) in 40% HBr in water (10 mL) was stirred for 2 h at 100° C. The reaction was cooled to room temperature. Then MeOH (10 mL) was added and stirred for overnight at 60° C. The solvent was removed. The residue was purified by reverse flash chromatography with the following conditions: column, C18 silica gel; mobile phase, MeCN in Water (10 mmol/L NH₄HCO₃), 20% to 50% gradient in 30 min; detector, UV 254 nm. The organic layer was concentrated under reduced pressure to afford the title compound (110 mg, 60.5% yield) as a yellow solid. LCMS (ESI): [M+H]⁺=313.1.

Step 5: tert-Butyl 4-(((3R,8aS)-7,7-difluoro-3-(3-hydroxy-4-(methoxycarbonyl)phenyl)hexahydropyrrolo[1,2-a]pyrazin-2 (1H)-yl)methyl)-5-methoxy-7-methyl-1H-indole-1-carboxylate. A mixture of methyl 4-((3R,8aS)-7,7-difluorooctahydropyrrolo[1,2-a]pyrazin-3-yl)-2-hydroxybenzoate (100 mg, 0.32 mmol), tert-butyl 4-formyl-5-methoxy-7-methylindole-1-carboxylate (140 mg, 0.48 mmol), AcOH (39 mg, 0.64 mmol) and NaBH(OAc)₃ (204 mg, 0.96 mmol) in dichloroethane (2 mL) was stirred for 4 h at 70° C. The resulting mixture was diluted with ethyl acatate. The residue was washed with water for 1 time. The organic layer was concentrated under reduced pressure. The residue was purified by silica gel column chromatography, eluted with petroleum ether/ethyl acetate (5:1) to afford the title compound (100 mg, 53.3% yield) as a light yellow solid. LCMS (ESI): [M+H]⁺=586.3.

Step 6: 4-((3R,8aS)-7,7-Difluoro-2-((5-methoxy-7-methyl-1H-indol-4-yl)methyl)octahydropyrrolo[1,2-a]pyrazin-3-yl)-2-hydroxybenzoic acid.

A mixture of tert-butyl 4-(((3R,8aS)-7,7-difluoro-3-(3-hydroxy-4-(methoxycarbonyl)phenyl)hexahydropyrrolo[1,2-a]pyrazin-2 (1H)-yl)methyl)-5-methoxy-7-methyl-1H-indole-1-carboxylate (70 mg, 0.12 mmol) and Potassium hydroxide (67 mg, 1.2 mmol) in water (0.2 mL) and Ethanol (2 mL) was stirred for 1 h at 70° C. The mixture was acidified to pH=6 with citric acid. The residue was purified by reversed-phase flash chromatography with the following conditions: column, C18 silica gel; mobile phase, MeCN in Water (10 mmol/L NH₄HCO₃), 10% to 35% gradient in 30 min; detector, UV 254 nm. The solvent was concentrated under reduced pressure to afford the title compound (16.3 mg, 28.9% yield) as a white solid. LCMS (ESI): [M+H]⁺=472.2. ¹H NMR (300 MHz, Methanol-d₄) δ 8.02 (d, J=7.9 Hz, 1H), 7.33 (d, J=3.1 Hz, 1H), 7.15 (d, J=1.7 Hz, 1H), 7.04 (d, J=6.0 Hz, 1H), 6.78 (s, 1H), 6.39 (d, J=3.1 Hz, 1H), 4.45 (d, J=12.3 Hz, 2H), 4.14 (d, J=12.5 Hz, 1H), 3.82 (s, 3H), 3.60-3.42 (m, 2H), 3.29 (d, J=3.4 Hz, 1H), 3.15 (t, J=11.6 Hz, 1H), 2.97-2.60 (m, 3H), 2.52 (s, 3H), 2.42-2.22 (m, 1H), 2.17-1.90 (m, 1H).

### Example 42. (S)-4-(1-((5-Methoxy-7-methyl-1H-indol-4-yl)methyl)-4-(3,3,3-trifluoropropyl)piperazin-2-yl)-2-(2-oxopyrrolidin-1-yl)benzoic acid & (R)-4-(1-((5-methoxy-7-methyl-1H-indol-4-yl)methyl)-4-(3,3,3-trifluoropropyl)piperazin-2-yl)-2-(2-oxopyrrolidin-1-yl)benzoic acid

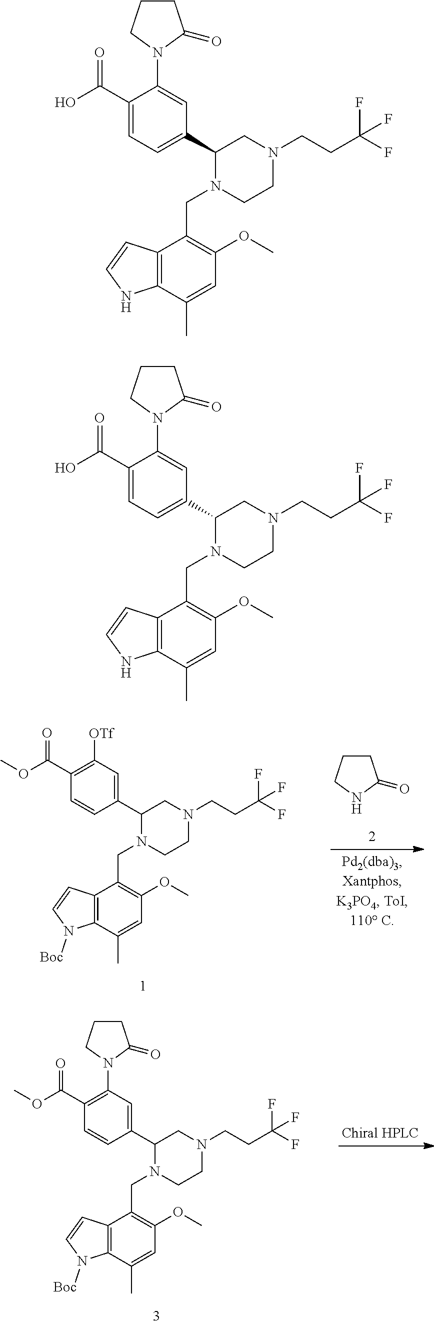

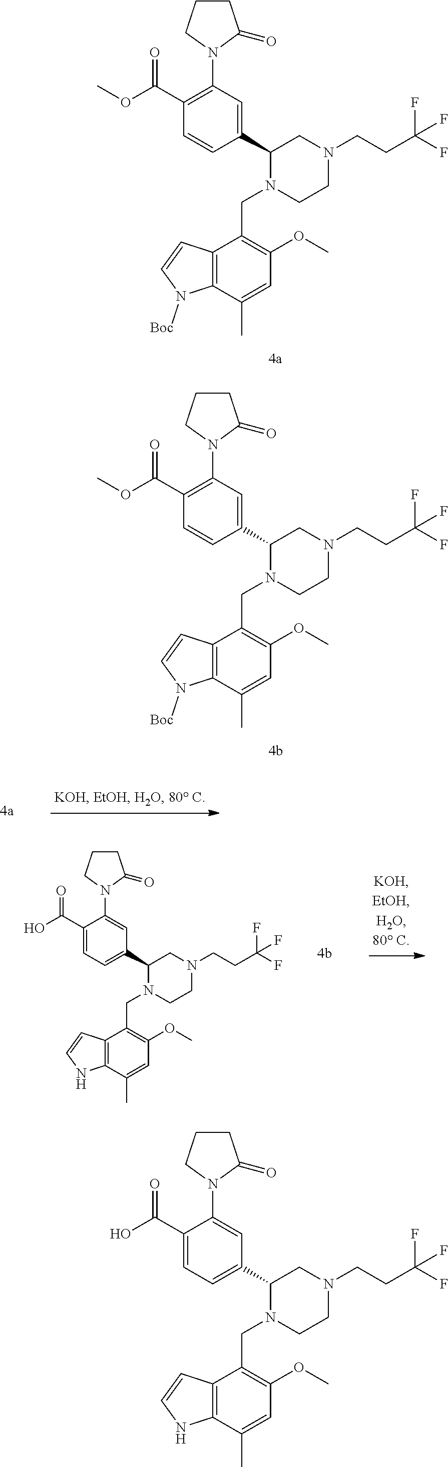 [SMILES: COC(=O)c1ccc([C@H]2CN(CCC(F)(F)F)CCN2Cc2c(OC)cc(C)c3c2ccn3C(=O)OC(C)(C)C)cc1N1CCCC1=O ; COC(=O)c1ccc([C@@H]2CN(CCC(F)(F)F)CCN2Cc2c(OC)cc(C)c3c2ccn3C(=O)OC(C)(C)C)cc1N1CCCC1=O ; COc1cc(C)c2[nH]ccc2c1CN1CCN(CCC(F)(F)F)C[C@@H]1c1ccc(C(=O)O)c(N2CCCC2=O)c1 ; COc1cc(C)c2[nH]ccc2c1CN1CCN(CCC(F)(F)F)C[C@H]1c1ccc(C(=O)O)c(N2CCCC2=O)c1]

Step 1: tert-Butyl 5-methoxy-4-((2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate. To a mixture of tert-butyl 5-methoxy-4-((2-(4-(methoxycarbonyl)-3-(trifluoromethanesulfonyloxy)phenyl)-4-(3,3,3-trifluoropropyl)piperazin-1-yl]methyl)-7-methylindole-1-carboxylate (350 mg, 0.47 mmol), pyrrolidone (175 mg, 2.06 mmol) and K₃PO₄ (320 mg, 1.51 mmol) in toluene (5 mL) were added XantPhos (70 mg, 0.12 mmol) and Pd₂(dba)₃ (55 mg, 0.06 mmol). After stirring for 1 h at 110° C. under a nitrogen atmosphere, the resulting mixture was extracted with ethyl acetate. The combined organic layers were washed with water, dried over anhydrous Na₂SO₄. After filtration, the filtrate was concentrated under reduced pressure. The residue was purified by reverse phase flash with the following conditions (acetonitrile, 5-80%/NH₄HCO₃ in water) to afford the title compound (240 mg, 75.19% yield) as a yellow oil. LCMS (ESI): [M+H]⁺=673.3.

Step 2: tert-Butyl 5-methoxy-4-(((2S)-2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (isomer 1, faster peak) & tert-butyl 5-methoxy-4-(((2R)-2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (isomer 2, slower peak).

The tert-butyl 5-methoxy-4-((2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (100 mg) was applied for further separation by Chiral HPLC.

tert-Butyl 5-methoxy-4-(((2S)-2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (isomer 1, faster peak) (36 mg, 36.00% yield) as a white solid. LCMS (ESI): [M+H]⁺=673.3. t_{R}=2.57 min (CHIRALPAK IE-3, 4.6*50 mm, 3 μm, (Hex:DCM=3:1) (0.1% DEA):IPA=90:10, 1.0 mL/min, 254 nm).

tert-Butyl 5-methoxy-4-(((2R)-2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (isomer 2, slower peak) (47 mg, 47.00% yield) as a white solid. LCMS (ESI): [M+H]⁺=673.3. t_{R}=3.32 min (CHIRALPAK IE-3, 4.6*50 mm, 3 μm, (Hex:DCM=3:1) (0.1% DEA):IPA=90:10, 1.0 mL/min, 254 nm).

Step 3: (S)-4-(1-((5-Methoxy-7-methyl-1H-indol-4-yl)methyl)-4-(3,3,3-trifluoropropyl)piperazin-2-yl)-2-(2-oxopyrrolidin-1-yl)benzoic acid & (R)-4-(1-((5-methoxy-7-methyl-1H-indol-4-yl)methyl)-4-(3,3,3-trifluoropropyl)piperazin-2-yl)-2-(2-oxopyrrolidin-1-yl)benzoic acid.

To a mixture of tert-butyl 5-methoxy-4-(((2S)-2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (36 mg, 0.05 mmol) and Potassium hydroxide (30 mg, 0.54 mmol) in water (1 mL) and Ethanol (1 mL) was stirred for 1 h at 60° C. The mixture was allowed to cool down to room temperature. The mixture was acidified to pH=6 with sat. citric acid. The residue was purified by reverse phase flash with the following conditions (acetonitrile, 5-30%/10 mM NH₄HCO₃ in water) to afford 4-((2S)-1-((5-methoxy-7-methyl-1H-indol-4-yl)methyl)-4-(3,3,3-trifluoropropyl)piperazin-2-yl)-2-(2-oxopyrrolidin-1-yl)benzoic acid (17.8 mg, 59.43% yield) as a white solid. LCMS (ESI): [M+H]⁺=559.2; ¹H NMR (300 MHz, Methanol-d₄) δ 7.94 (d, J=8.0 Hz, 1H), 7.61 (d, J=8.0 Hz, 1H), 7.53 (s, 1H), 7.25 (d, J=3.1 Hz, 1H), 6.72 (s, 1H), 6.37 (d, J=3.1 Hz, 1H), 4.13 (d, J=12.3 Hz, 1H), 4.07-3.98 (m, 1H), 3.97-3.89 (m, 2H), 3.77 (s, 4H), 3.16 (d, J=12.1 Hz, 1H), 3.04 (d, J=11.8 Hz, 1H), 2.99-2.78 (m, 2H), 2.75-2.60 (m, 2H), 2.60-2.50 (m, 2H), 2.49-2.46 (m, 3H), 2.46-2.31 (m, 4H), 2.30-2.15 (m, 2H).

To a mixture of tert-butyl 5-methoxy-4-(((2R)-2-(4-(methoxycarbonyl)-3-(2-oxopyrrolidin-1-yl)phenyl)-4-(3,3,3-trifluoropropyl)piperazin-1-yl)methyl)-7-methylindole-1-carboxylate (47 mg, 0.07 mmol) and Potassium hydroxide (39 mg, 0.70 mmol) in water (1 mL) and Ethanol (1 mL) was stirred for 1 h at 60° C. The mixture was allowed to cool down to room temperature. The mixture was acidified to pH=6 with sat. citric acid. The residue was purified by reverse phase flash with the following conditions (acetonitrile, 5-30%/10 mM NH₄HCO₃ in water) to afford 4-((2R)-1-((5-methoxy-7-methyl-1H-indol-4-yl)methyl)-4-(3,3,3-trifluoropropyl)piperazin-2-yl)-2-(2-oxopyrrolidin-1-yl)benzoic acid (17.4 mg, 44.54% yield) as a white solid. LCMS (ESI): [M+H]⁺=559.2; ¹H NMR (300 MHz, Methanol-d₄) δ 7.93 (d, J=7.9 Hz, 1H), 7.60 (d, J=8.0 Hz, 1H), 7.52 (s, 1H), 7.24 (d, J=3.1 Hz, 1H), 6.71 (s, 1H), 6.37 (d, J=3.2 Hz, 1H), 4.11 (d, J=12.3 Hz, 1H), 4.05-3.85 (m, 3H), 3.77 (s, 3H), 3.71 (s, 1H), 3.14 (d, J=12.1 Hz, 1H), 3.03 (d, J=12.2 Hz, 1H), 2.98-2.76 (m, 2H), 2.73-2.60 (m, 2H), 2.54 (t, J=8.0 Hz, 2H), 2.47 (d, J=0.8 Hz, 3H), 2.46-2.30 (m, 4H), 2.30-2.15 (m, 2H).

### Example 43. (S)-8-(4-(2,2-Difluoroethyl)-1-((5-methoxy-7-methyl-1H-indol-4-yl)methyl)piperazin-2-yl)-3,4-dihydro-2H-benzo[b][1,4]oxazine-5-carboxylic acid & (R)-8-(4-(2,2-difluoroethyl)-1-((5-methoxy-7-methyl-1H-indol-4-yl)methyl)piperazin-2-yl)-3,4-dihydro-2H-benzo[b][1,4]oxazine-5-carboxylic acid

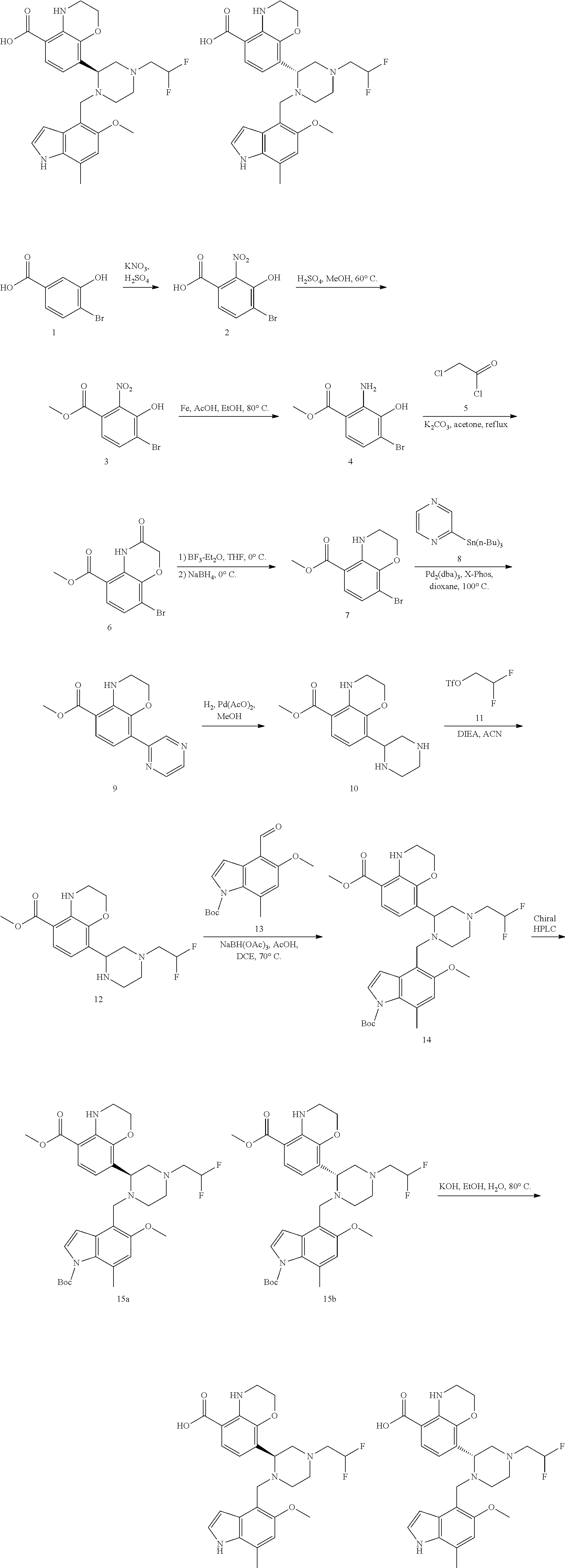

Step 1: 4-Bromo-3-hydroxy-2-nitrobenzoic acid. To a stirred mixture of 4-bromo-3-hydroxybenzoic acid (10.00 g, 46.08 mmol) in H₂SO₄ (100 mL) were added KNO₃ (4.20 g, 41.54 mmol) in portions at 0° C. The resulting mixture was stirred for 1 h at 0° C. The resulting mixture was extracted with ethyl acetate. The combined organic layers were washed with brine, dried over anhydrous Na₂SO₄. After filtration, the filtrate was concentrated under reduced pressure to afford 4-bromo-3-hydroxy-2-nitrobenzoic acid (12.00 g, 87.7% yield) as a brown solid. LCMS (ESI): [M+H]⁺=262.0.

Step 2: Methyl 4-bromo-3-hydroxy-2-nitrobenzoate. To a stirred mixture of 4-bromo-3-hydroxy-2-nitrobenzoic acid (13.00 g, 49.61 mmol) in MeOH (100 mL) was added con. H₂SO₄ (50 mL, 938.10 mmol) dropwise at 0° C. The resulting mixture was stirred for 2 h at 60° C. The resulting mixture was diluted with ice water. The resulting mixture was extracted with ethyl acetate. The combined organic layers were washed with water dried over anhydrous Na₂SO₄. After filtration, the filtrate was concentrated under reduced pressure to afford methyl 4-bromo-3-hydroxy-2-nitrobenzoate (13.00 g, 94.9% yield) as a brown solid. LCMS (ESI): [M+H]⁺=275.9.

Step 3: Methyl 2-amino-4-bromo-3-hydroxybenzoate. A solution of methyl 4-bromo-3-hydroxy-2-nitrobenzoate (12.00 g, 43.47 mmol) in Ethanol (5 mL, 17.21 mmol) was added Fe (12.14 g, 217.36 mmol) and HOAc (6.00 mL, 104.76 mmol) and stirred for 1 h at 80° C. The solids were filtered out and the solvent was concentrated under vacuum to afford the product. The residue was purified by flash chromatography on silica gel eluting with petroleum ether/ethyl acetate (0-29%) to afford the title compound (6.90 g, 64.5% yield) as a yellow solid. LCMS (ESI): [M+H]⁺=248.0.

Step 4: Methyl 8-bromo-3-oxo-2,4-dihydro-1,4-benzoxazine-5-carboxylate. A solution of methyl 2-amino-4-bromo-3-hydroxybenzoate (5.90 g, 23.98 mmol) in acetone (30 mL) was added chloroacetyl chloride (5.42 g, 47.96 mmol) and K₂CO₃ (9.94 g, 71.93 mmol) and stirred for 12 hours at 60° C. The solvent was concentrated under vacuum. The residue was purified by flash chromatography on silica gel eluting with petroleum ether/ethyl acetate (0-40%) to afford the title compound (3.24 g, 47.2% yield) as a yellow solid. LCMS (ESI): [M+H]⁺=288.0.

Step 5: Methyl 8-bromo-3,4-dihydro-2H-1,4-benzoxazine-5-carboxylate. To a stirred mixture of methyl 8-bromo-3-oxo-2,4-dihydro-1,4-benzoxazine-5-carboxylate (2.40 g, 8.39 mmol) in THF (20 mL) was added BF₃·Et₂O (2.38 g, 16.78 mmol) dropwise at 0° C. and stirred for 20 mins. To the above mixture was added NaBH₄ (0.79 g, 20.97 mmol) in portions at 0° C. The resulting mixture was stirred for additional 1.5 hours at 0° C. The resulting mixture was diluted with ethyl acetate. The reaction was quenched with 1 M HCl. The mixture was neutralized to pH=7 with saturated Na₂CO₃ (aq.). The resulting mixture was extracted with ethyl acetate. The combined organic layers were washed with brine, dried over anhydrous Na₂SO₄. After filtration, the filtrate was concentrated under reduced pressure. The residue was purified by silica gel column chromatography, eluted with ethyl acetate/petroleum ether (0-30%) to afford the title compound (1.20 g, 52.5% yield) as a yellow solid. LCMS (ESI): [M+H]⁺=274.0.

Step 6: Methyl 8-(pyrazin-2-yl)-3,4-dihydro-2H-1,4-benzoxazine-5-carboxylate. A solution of methyl 8-bromo-3,4-dihydro-2H-1,4-benzoxazine-5-carboxylate (1.20 g, 4.41 mmol) in 1,4-dioxane (20 mL) was added 2-(tributylstannyl)pyrazine (1.95 g, 5.29 mmol) and Pd₂(dba)₃ (0.40 g, 0.44 mmol) and Xphos (0.21 g, 0.44 mmol) and was stirred for 1 h at 100° C. under nitrogen atmosphere. The reaction was quenched with KF (aq) at room temperature. The resulting mixture was diluted with ethyl acetate. The resulting mixture was washed with water. The organic layer was concentrated under reduced pressure. The crude product was purified by flash chromatography on silica gel eluting with petroleum ether/ethyl acetate (0-65%) to afford the title compound (960 mg, 80.2% yield) as a yellow solid. LCMS (ESI): [M+H]⁺=272.1.

Step 7: Methyl 8-(piperazin-2-yl)-3,4-dihydro-2H-1,4-benzoxazine-5-carboxylate. A solution of methyl 8-(pyrazin-2-yl)-3,4-dihydro-2H-1,4-benzoxazine-5-carboxylate (1.20 g, 4.42 mmol) in MeOH (30 mL) was added Palladium acetate (0.50 g, 2.21 mmol) at room temperature. The mixture was stirred for 12 h at room temperature under hydrogen, after filtered through a Celite pad and concentrated under reduced pressure to afford the title compound (1.5 g, 88.5% yield) as a yellow solid. LCMS (ESI): [M+H]⁺=278.0.

Step 8: Methyl 8-(4-(2,2-difluoroethyl)piperazin-2-yl)-3,4-dihydro-2H-1,4-benzoxazine-5-carboxylate. A solution of methyl 8-(piperazin-2-yl)-3,4-dihydro-2H-1,4-benzoxazine-5-carboxylate (400 mg, 1.44 mmol) in ACN (10 mL) was added 2,2-difluoroethyl trifluoromethanesulfonate (216 mg, 1.01 mmol) and N,N-diisopropylethylamine (559 mg, 4.32 mmol) and stirred for 6 hours at room temperature. The solvent was concentrated under vacuum. The residue was purified by flash chromatography on silica gel eluting with dichloromethane/methanol (0-10%) to afford the title compound (490 mg, 99.5% yield) as a yellow solid. LCMS (ESI): [M+H]⁺=341.9.

Step 9: Methyl 8-(1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate. A solution of methyl 8-(4-(2,2-difluoroethyl)piperazin-2-yl)-3,4-dihydro-2H-1,4-benzoxazine-5-carboxylate (240 mg, 0.70 mmol) in DCE (3 mL) was added tert-butyl 4-formyl-5-methoxy-7-methylindole-1-carboxylate (244 mg, 0.84 mmol), HOAc (42 mg, 0.70 mmol) and NaBH(OAc)₃ (298 mg, 1.41 mmol) at room temperature. The resulting mixture was stirred for overnight at 70° C. The solvent was concentrated. The rsidue was purified by flash with petroleum ether/ethyl acetate (0-30%) to afford the title compound (200 mg, 46.3% yield) as a yellow solid. LCMS (ESI): [M+H]⁺=615.4.

Step 10: Methyl 8-((2S)-1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (isomer 1, faster peak).

Methyl 8-((2R)-1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (isomer 2, slower peak).

Methyl 8-(1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (200 mg) was applied for further separation by Chiral HPLC.

Methyl 8-((2S)-1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (isomer 1, faster peak) (50 mg, 25% yield), LCMS (ESI): [M+H]⁺=615.4, t_{R}=3.23 min (CHIRAL Cellulose-SB, 4.6*50 mm, 3.5 μm, Hex(0.1% DEA):IPA=95:5, 1.0 mL/min, 254 nm).

Methyl 8-((2R)-1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (isomer 2, slower peak) (40 mg, 20% yield), LCMS (ESI): [M+H]⁺=615.4, t_{R}=3.68 min (CHIRAL Cellulose-SB, 4.6*50 mm, 3.5 μm, Hex(0.1% DEA):IPA=95:5, 1.0 mL/min, 254 nm).

Step 11: (S)-8-(4-(2,2-Difluoroethyl)-1-((5-methoxy-7-methyl-1H-indol-4-yl)methyl)piperazin-2-yl)-3,4-dihydro-2H-benzo[b][1,4]oxazine-5-carboxylic acid & (R)-8-(4-(2,2-difluoroethyl)-1-((5-methoxy-7-methyl-1H-indol-4-yl)methyl)piperazin-2-yl)-3,4-dihydro-2H-benzo[b][1,4]oxazine-5-carboxylic acid.

A solution of methyl 8-((2S)-1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (50 mg, 0.08 mmol) in Ethanol (1.5 mL) was added Potassium hydroxide (46 mg, 0.81 mmol) in water (0.3 mL) and stirred for 1.5 hours at 70° C. The mixture was allowed to cool down to room temperature and was neutralized to pH 7 with sat. Citric acid. The resulting residue was purified by reverse phase chromatography (Acetonitrile 0-30%/0.1% Ammonium bicarbonate in water) to afford 8-((2S)-4-(2,2-difluoroethyl)-1-((5-methoxy-7-methyl-1H-indol-4-yl)methyl)piperazin-2-yl)-3,4-dihydro-2H-1,4-benzoxazine-5-carboxylic acid (25.2 mg, 61.46% yield) as a white solid. LCMS (ESI): [M+H]⁺=501.1. ¹H NMR (300 MHz, DMSO-d₆) δ 10.79 (s, 1H), 7.43 (d, J=8.3 Hz, 1H), 7.23 (t, J=2.8 Hz, 1H), 6.89 (d, J=8.4 Hz, 1H), 6.64 (s, 1H), 6.50-6.47 (m, 1H), 6.31-5.90 (m, 1H), 4.17 (d, J=5.6 Hz, 2H), 3.70 (s, 5H), 3.47 (s, 2H), 3.20 (d, J=11.9 Hz, 1H), 2.78-2.55 (m, 5H), 2.41 (s, 3H), 2.24-2.05 (m, 3H).

A solution of methyl 8-((2R)-1-((1-(tert-butoxycarbonyl)-5-methoxy-7-methylindol-4-yl)methyl)-4-(2,2-difluoroethyl)piperazin-2-yl)-3,4-dihydro-2H-1,4-benzoxazine-5-carboxylate (40 mg, 0.06 mmol) in Ethanol (1.5 mL) was added Potassium hydroxide (36 mg, 0.650 mmol) in water (0.3 mL) and stirred for 1.5 hours at 70° C. The mixture was allowed to cool down to room temperature and was neutralized to pH 7 with sat. Citric acid. The resulting residue was purified by reverse phase chromatography (Acetonitrile 0-30%/0.1% Ammonium bicarbonate in water) to afford 8-((2R)-4-(2,2-difluoroethyl)-1-((5-methoxy-7-methyl-1H-indol-4-yl)methyl)piperazin-2-yl)-3,4-dihydro-2H-1,4-benzoxazine-5-carboxylic acid (17.1 mg, 51.76% yield) as a white solid. LCMS (ESI): [M+H]⁺=501.1. ¹H NMR (300 MHz, DMSO-d₆) δ 10.79 (s, 1H), 7.43 (d, J=8.3 Hz, 1H), 7.23 (t, J=2.8 Hz, 1H), 6.89 (d, J=8.4 Hz, 1H), 6.64 (s, 1H), 6.50-6.47 (m, 1H), 6.31-5.90 (m, 1H), 4.17 (d, J=5.6 Hz, 2H), 3.70 (s, 5H), 3.47 (s, 2H), 3.20 (d, J=11.9 Hz, 1H), 2.78-2.55 (m, 5H), 2.41 (s, 3H), 2.24-2.05 (m, 3H).

### Example 44. (S)-3-Amino-5-(4-(2,2-difluoroethyl)-1-((5-methoxy-7-methyl-1H-indol-4-yl)methyl)piperazin-2-yl) picolinic acid & (R)-3-Amino-5-(4-(2,2-difluoroethyl)-1-((5-methoxy-7-methyl-1H-indol-4-yl)methyl)piperazin-2-yl) picolinic acid

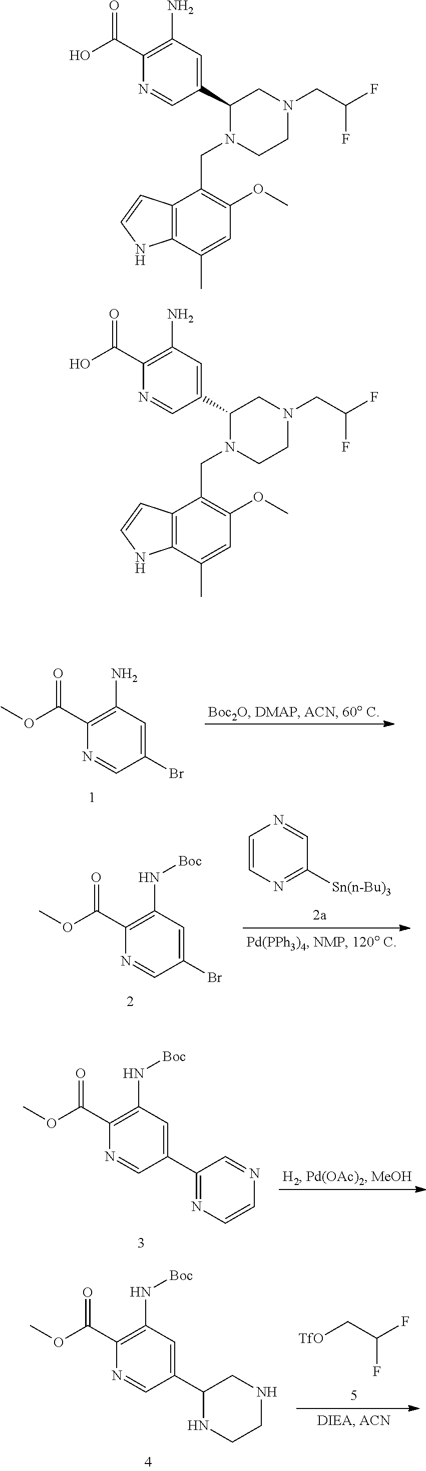

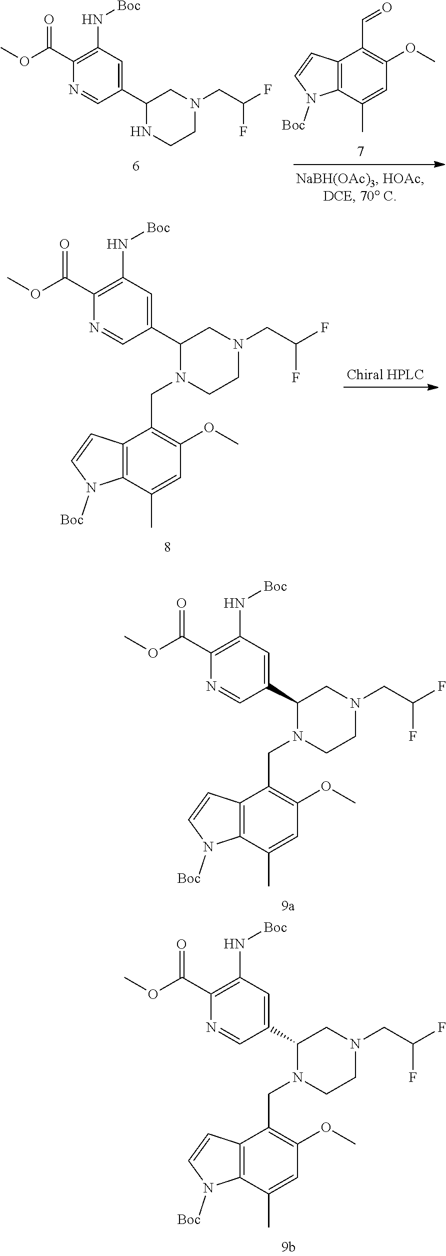

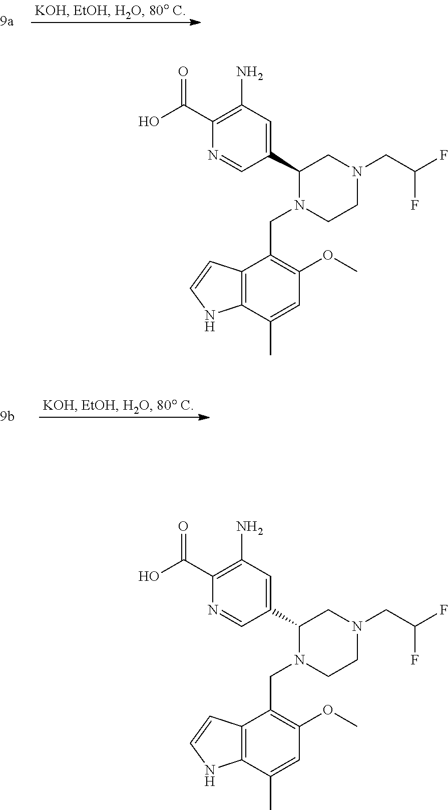 [SMILES: COc1cc(C)c2[nH]ccc2c1CN1CCN(CC(F)F)C[C@@H]1c1cnc(C(=O)O)c(N)c1 ; COc1cc(C)c2[nH]ccc2c1CN1CCN(CC(F)F)C[C@H]1c1cnc(C(=O)O)c(N)c1]

Step 1: Methyl 3-(bis(tert-butoxycarbonyl)amino)-5-bromopyridine-2-carboxylate To a mixture of methyl 3-amino-5-bromopyridine-2-carboxylate (2.0 g, 8.66 mmol) and Boc₂O (7.5 g, 34.36 mmol) in acetonitrile (30 mL) was added DMAP (232 mg, 1.90 mmol) and stirred for 1 h at 60° C. The resulting mixture was extracted with ethyl acetate. The combined organic layers were washed with brine, dried over anhydrous Na₂SO₄. After filtration, the filtrate was concentrated under reduced pressure. The residue was purified by silica gel column chromatography, eluted with petroleum ether/ethyl acetate (4:1) to afford the title compound (3.0 g, 80.36% yield) as a white solid. LCMS (ESI): [M+H]⁺=431.3.

Step 2: Methyl 3-((tert-butoxycarbonyl)amino)-5-(pyrazin-2-yl)pyridine-2-carboxylate. To a mixture of methyl 3-(bis(tert-butoxycarbonyl)amino)-5-bromopyridine-2-carboxylate (3.0 g, 6.96 mmol) and 2-(tributylstannyl)pyrazine (3.2 g, 8.67 mmol) in NMP (50 mL) were added Pd(PPh₃)₄ (1.7 g, 1.47 mmol) and stirring for 5 h at 120° C. under a nitrogen atmosphere. The reaction was quenched with sat. KF (aq.) at room temperature. The resulting mixture was extracted with ethyl acetate. The combined organic layers were washed with brine, dried over anhydrous Na₂SO₄. After filtration, the filtrate was concentrated under reduced pressure. The residue was purified by silica gel column chromatography, eluted with petroleum ether/ethyl acetate (2:3) to afford the title compound (2.2 g, 95.74% yield) as a yellow solid. LCMS (ESI): [M+H]⁺=331.1.

Step 3: Methyl 3-((tert-butoxycarbonyl)amino)-5-(piperazin-2-yl)pyridine-2-carboxylate. To a mixture of methyl 3-((tert-butoxycarbonyl)amino)-5-(pyrazin-2-yl)pyridine-2-carboxylate (2.2 g, 6.66 mmol) and Pd(OAc)₂ (2.2 g, 9.80 mmol) in MeOH (50 mL) was stirred for 5 h at room temperature under hydrogen atmosphere. The resulting mixture was filtered, the filter cake was washed with MeOH. The filtrate was concentrated under reduced pressure to afford the title compound (2.2 g, 98.20% yield) as a black solid. LCMS (ESI): [M+H]⁺=337.2.

Step 4: Methyl 3-((tert-butoxycarbonyl)amino)-5-(4-(2,2-difluoroethyl)piperazin-2-yl)pyridine-2-carboxylate. To a stirred mixture of methyl 3-((tert-butoxycarbonyl)amino)-5-(piperazin-2-yl)pyridine-2-carboxylate (1.0 g, 2.97 mmol) and DIEA (1.9 g, 14.70 mmol) in acetonitrile (30 mL) was added 2,2-difluoroethyl trifluoromethanesulfonate (1.0 g, 4.67 mmol) in portions at room temperature. The resulting mixture was stirred for 2 h at room temperature. The residue was purified by reverse phase flash with the following conditions (acetonitrile, 5-55%/water) to afford the title compound (460 mg, 38.64% yield) as a yellow oil. LCMS (ESI): [M+H]⁺=401.2.

Step 5: tert-Butyl 4-((2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate. To a stirred mixture of methyl 3-((tert-butoxycarbonyl)amino)-5-(4-(2,2-difluoroethyl)piperazin-2-yl)pyridine-2-carboxylate (390 mg, 0.97 mmol), tert-butyl 4-formyl-5-methoxy-7-methylindole-1-carboxylate (390 mg, 1.35 mmol) and HOAc (120 mg, 1.99 mmol) in 1,2-dichloroethane (4 mL) were added NaBH(OAc)₃ (826 mg, 3.90 mmol) in portions at room temperature. The resulting mixture was stirred for 6 h at 70° C. The resulting mixture was extracted with ethyl acetate. The combined organic layers were washed with water, dried over anhydrous Na₂SO₄. After filtration, the filtrate was concentrated under reduced pressure. The residue was purified by silica gel column chromatography, eluted with petroleum ether/ethyl acetate (1:1) to afford the title compound (370 mg, 56.38% yield) as a yellow oil. LCMS (ESI): [M+H]⁺=674.3.

Step 6: tert-Butyl 4-(((2S)-2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate (isomer 1, faster peak) & tert-butyl 4-(((2R)-2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate (isomer 2, slower peak).

The tert-butyl 4-((2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate (180 mg) was applied for further separation by Chiral HPLC with the folloing condition: Column: CHIRAL ART Amylose-SA, 2*25 cm, 5 μm; Mobile Phase A: Hex(0.5% of 2 M NH₃-MeOH)-HPLC, Mobile Phase B: IPA-HPLC; Flow rate: 20 mL/min; Gradient: 2% B to 2% B in 18 min; Wave Length: 220/254 nm; RT1(min): 10.898; RT2(min): 14.892; Sample Solvent: Ethanol-HPLC; Injection Volume: 0.6 mL; Number of Runs: 9.

tert-Butyl 4-(((2S)-2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate (isomer 1, faster peak) (65 mg, 36.11% yield) as a white solid. LCMS (ESI): [M+H]⁺=674.3. t_{R}=2.20 min, (CHIRALPAK IA-3, 4.6*50 mm, 3 μm, Hex(0.1% DEA):IPA=98:2, 1.0 mL/min, 254 nm).

tert-Butyl 4-(((2R)-2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate (isomer 2, slower peak) (70 mg, 38,89% yield) as a white solid. LCMS (ESI): [M+H]⁺=674.3. t_{R}=3.23 min, (CHIRALPAK IA-3, 4.6*50 mm, 3 μm, Hex(0.1% DEA):IPA=98:2, 1.0 mL/min, 254 nm).

Step 7: (S)-3-Amino-5-(4-(2,2-difluoroethyl)-1-((5-methoxy-7-methyl-1H-indol-4-yl)methyl)piperazin-2-yl) picolinic acid & (R)-3-Amino-5-(4-(2,2-difluoroethyl)-1-((5-methoxy-7-methyl-1H-indol-4-yl)methyl)piperazin-2-yl) picolinic acid. To a mixture of tert-butyl 4-(((2S)-2-(5-((tert-butoxycarbonyl)amino)-6-(methoxycarbonyl)pyridin-3-yl)4-(2,2-difluoroethyl)piperazin-1-yl)methyl)5-methoxy-7-methylindole-1-carboxylate (65 mg, 0.10 mmol) and Potassium hydroxide (55 mg, 0.98 mmol) in water (1 mL) and Ethanol (1.5 mL) was stirred for 4 h at 80° C. The mixture was allowed to cool down to room temperature. The mixture was acidified to pH=6 with sat. citric acid. The residue was purified by reverse phase flash with the following conditions (acetonitrile, 5-30%/10 mM NH₄HCO₃ in water) to afford 3-amino-5-((2S)-4-(2,2-difluoroethyl)-1-((5-methoxy-7-methyl-1H-indol-4-yl)methyl)piperazin-2-yl)pyridine-2-carboxylic acid (21.3 mg, 48.05% yield) as a light yellow solid. LCMS (ESI): [M+H]⁺=460.2; ¹H NMR (300 MHz, DMSO-d₆) δ 10.84 (d, J=2.8 Hz, 1H), 8.02 (s, 1H), 7.43 (s, 1H), 7.25 (t, J=2.8 Hz, 1H), 6.66 (s, 1H), 6.43 (t, J=2.4 Hz, 1H), 6.36-5.87 (m, 1H), 3.71 (s, 3H), 3.60 (d, J=11.8 Hz, 1H), 3.41-3.30 (m, 1H), 3.27 (d, J=11.8 Hz, 1H), 2.83 (d, J=11.2 Hz, 1H), 2.79-2.57 (m, 4H), 2.42 (s, 3H), 2.33-2.11 (m, 3H).

To a mixture of tert-butyl 4-(((2R)-2-(5-((tert-butoxycarbonyl)amino)-6-(methoxycarbonyl)pyridin-3-yl)-4-(2,2-difluoroethyl)piperazin-1-yl)methyl)-5-methoxy-7-methylindole-1-carboxylate (65 mg, 0.10 mmol) and Potassium hydroxide (55 mg, 0.98 mmol) in water (1 mL) and Ethanol (1.5 mL) was stirred for 4 h at 80° C. The mixture was allowed to cool down to room temperature. The mixture was acidified to pH=6 with sat. citric acid. The residue was purified by reverse phase flash with the following conditions (acetonitrile, 5-30%/10 mM NH₄HCO₃ in water) to afford 3-amino-5-((2R)-4-(2,2-difluoroethyl)-1-((5-methoxy-7-methyl-1H-indol-4-yl)methyl)piperazin-2-yl)pyridine-2-carboxylic acid (26.0 mg, 58.65% yield) as a light yellow solid. LCMS (ESI): [M+H]⁺=460.2; ¹H NMR (300 MHz, DMSO-d₆) δ 10.84 (s, 1H), 8.00 (s, 1H), 7.40 (s, 1H), 7.25 (t, J=2.8 Hz, 1H), 6.66 (s, 1H), 6.51-6.36 (m, 1H), 6.35-5.85 (m, 1H), 3.71 (s, 3H), 3.61 (d, J=11.8 Hz, 1H), 3.32-3.18 (m, 2H), 2.83 (d, J=11.1 Hz, 1H), 2.79-2.57 (m, 4H), 2.42 (s, 3H), 2.35-2.09 (m, 3H).

### Example 45. 8-(4-(2,2-Difluoroethyl)-1-((5-methoxy-7-methyl-1H-indol-4-yl)methyl)piperazin-2-yl)-3-oxo-3,4-dihydro-2H-benzo[b][1,4]oxazine-5-carboxylic acid

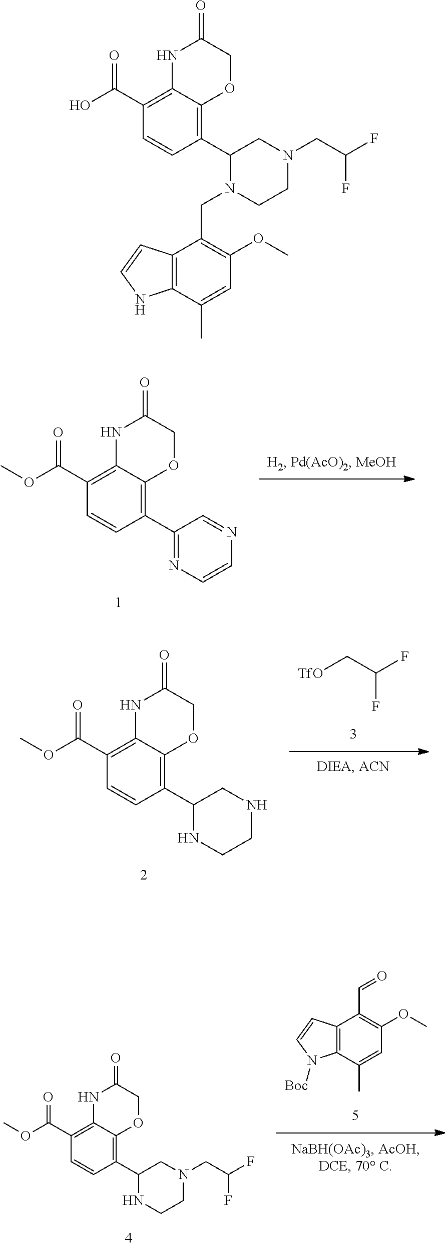 [SMILES: COc1cc(C)c2[nH]ccc2c1CN1CCN(CC(F)F)CC1c1ccc(C(=O)O)c2c1OCC(=O)N2 ; COC(=O)c1ccc(-c2cnccn2)c2c1NC(=O)CO2 ; COC(=O)c1ccc(C2CNCCN2)c2c1NC(=O)CO2 ; O=S(=O)(OCC(F)F)C(F)(F)F ; COC(=O)c1ccc(C2CN(CC(F)F)CCN2)c2c1NC(=O)CO2 ; COc1cc(C)c2c(ccn2C(=O)OC(C)(C)C)c1C=O]

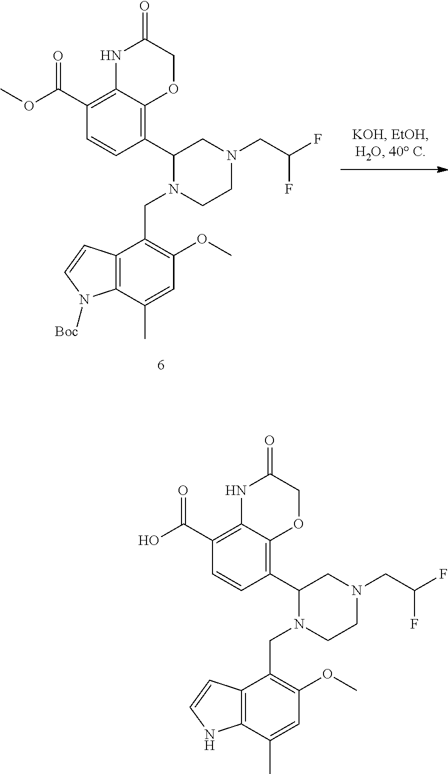 [SMILES: COC(=O)c1ccc(C2CN(CC(F)F)CCN2Cc2c(OC)cc(C)c3c2ccn3C(=O)OC(C)(C)C)c2c1NC(=O)CO2 ; COc1cc(C)c2[nH]ccc2c1CN1CCN(CC(F)F)CC1c1ccc(C(=O)O)c2c1OCC(=O)N2]

Step 1: Methyl 3-oxo-8-(piperazin-2-yl)-2,4-dihydro-1,4-benzoxazine-5-carboxylate. To a stirred mixture of methyl 3-oxo-8-(pyrazin-2-yl)-2,4-dihydro-1,4-benzoxazine-5-carboxylate (300 mg, 1.05 mmol) in MeOH (3 mL) was added Pd(OAc)₂ (90 mg, 0.40 mmol). The resulting mixture was stirred for 2 h at room temperature under hydrogen atmosphere. The solids were filtered out, the filtrate was concentrated under reduced pressure to afford the title compound (200 mg, 65.3% yield) as a white solid. LCMS (ESI): [M+H]⁺=292.1.

Step 2: Methyl 8-(4-(2,2-difluoroethyl)piperazin-2-yl)-3-oxo-3,4-dihydro-2H-benzo[b][1,4]oxazine-5-carboxylate. To a stirred mixture of methyl 3-oxo-8-(piperazin-2-yl)-2,4-dihydro-1,4-benzoxazine-5-carboxylate (200 mg, 0.69 mmol) and N,N-diisopropylethylamine (260 mg, 2.01 mmol) in ACN (3 mL) was added 2,2-difluoroethyl trifluoromethanesulfonate (180 mg, 0.84 mmol). The resulting mixture was stirred for 2 h at room temperature. The solvent was removed. The residue was purified by silica gel column chromatography, eluted with dichloromethane/methanol (9:1) to afford the title compound (130 mg, 53.3% yield) as a yellow oil. LCMS (ESI) [M+H]⁺=356.1.

Step 3: Methyl 8-(1-((1-(tert-butoxycarbonyl)-5-methoxy-7-methyl-1H-indol-4-yl)methyl)-4-(2,2-difluoroethyl)piperazin-2-yl)-3-oxo-3,4-dihydro-2H-benzo[b][1,4]oxazine-5-carboxylate.

To a stirred mixture of methyl 8-(4-(2,2-difluoroethyl)piperazin-2-yl)-3-oxo-3,4-dihydro-2H-benzo[b][1,4]oxazine-5-carboxylate (130 mg, 0.37 mmol) and tert-butyl 4-formyl-5-methoxy-7-methylindole-1-carboxylate (155 mg, 0.54 mmol) in dichloroethane (3 mL) was added AcOH (26 mg, 0.43 mmol) and Sodium triacetoxyborohydride (155 mg, 0.73 mmol) in portions. The resulting mixture was stirred for additional 5 h at 70° C. The resulting mixture was concentrated under reduced pressure. The residue was purified by reverse flash chromatography with the following conditions: column, C18 silica gel; mobile phase, MeCN in Water (10 mmol/L NH4HCO₃), 60% to 100% gradient in 30 min; detector, UV 254 nm. to afford the title compound (120 mg, 52.2% yield) as a yellow oil. LCMS (ESI) [M+H]⁺=629.3.

Step 4: 8-(4-(2,2-Difluoroethyl)-1-((5-methoxy-7-methyl-1H-indol-4-yl)methyl)piperazin-2-yl)-3-oxo-3,4-dihydro-2H-benzo[b][1,4]oxazine-5-carboxylic acid. To a stirred mixture of methyl 8-(1-((1-(tert-butoxycarbonyl)-5-methoxy-7-methyl-1H-indol-4-yl)methyl)-4-(2,2-difluoroethyl)piperazin-2-yl)-3-oxo-3,4-dihydro-2H-benzo[b][1,4]oxazine-5-carboxylate (120 mg, 0.19 mmol) in water (0.6 mL) and Ethanol (3 mL) was added Potassium hydroxide (216 mg, 3.85 mmol). The resulting mixture was stirred for 2 h at 40° C. The reaction mixture was acidified to pH=6 with sat. citric acid solution. The mixture was purified by reverse flash chromatography with the following conditions: column, C18 silica gel; mobile phase, MeCN in Water (10 mmol/L NH4HCO₃), 10% to 30% gradient in 30 min; detector, UV 254 nm. to afford the title compound (24.3 mg, 24.7% yield) as a white solid. LCMS (ESI) [M+H]⁺=515.2. ¹H NMR (300 MHz, DMSO-d₆) δ 11.00 (s, 1H), 10.85 (s, 1H), 7.69 (d, J=8.3 Hz, 1H), 7.47 (d, J=8.3 Hz, 1H), 7.26 (t, J=2.8 Hz, 1H), 6.66 (s, 1H), 6.46-6.41 (m, 1H), 6.32-5.89 (m, 1H), 4.75 (s, 2H), 3.92 (s, 1H), 3.71 (s, 4H), 3.56-3.42 (m, 1H), 3.23-3.10 (m, 1H), 2.88-2.59 (m, 5H), 2.42 (s, 3H), 2.21 (s, 2H).

### Example 46. 4-(4-(2,2-Difluoroethyl)-1-((5-methoxy-7-methyl-1H-indol-4-yl)methyl)piperazin-2-yl)-2-(methylamino)benzoic acid

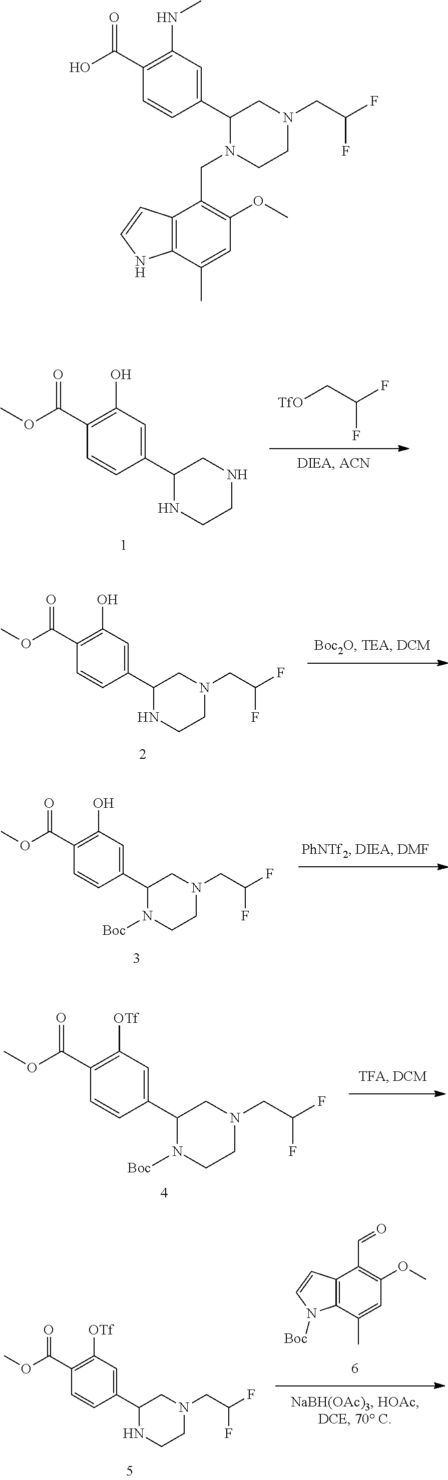

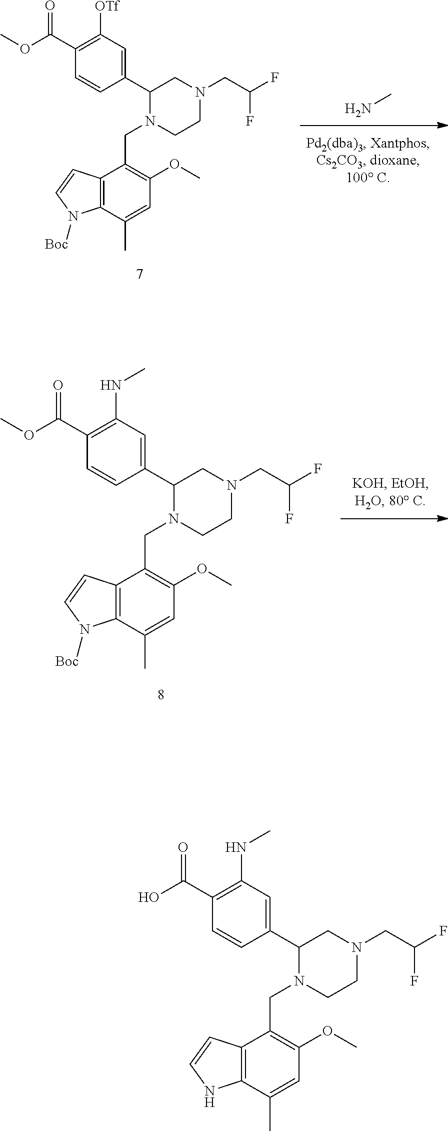

Step 1: Methyl 4-(4-(2,2-difluoroethyl)piperazin-2-yl)-2-hydroxybenzoate. To a stirred solution of methyl 2-hydroxy-4-(piperazin-2-yl)benzoate (2.5 g, 10.58 mmol) and 2,2-difluoroethyl trifluoromethanesulfonate (2.95 g, 13.75 mmol) in ACN (15 mL) was added N,N-diisopropylethylamine (4.10 g, 31.74 mmol) in portions at room temperature. The resulting mixture was stirred for additional 2 h at room temperature. The solvent was removed and the residue was purified by silica gel column chromatography, eluted with dichloromethane/methanol (1:19) to afford the title compound (3 g, 94.41% yield) as a yellow oil. LCMS (ESI): [M+H]⁺=301.1.

Step 2: tert-Butyl 4-(2,2-difluoroethyl)-2-(3-hydroxy-4-(methoxycarbonyl)phenyl)piperazine-1-carboxylate. To a stirred mixture of methyl 4-(4-(2,2-difluoroethyl)piperazin-2-yl)-2-hydroxybenzoate (3 g, 9.99 mmol) and Di-tert-butyl dicarbonate (4.32 g, 19.78 mmol) in dichloromethane (20 mL) were added Triethylamine (1.98 g, 19.58 mmol) in portions. The resulting mixture was stirred for additional 2 h at room temperature. The solvent was removed under vacuum. The residue was purified by silica gel column chromatography, eluted with petroleum ether/ethyl acetate (1:9) to afford the title compound (2 g, 50.00% yield) as a yellow oil. LCMS (ESI): [M+H]⁺=401.2.

Step 3: tert-Butyl 4-(2,2-difluoroethyl)-2-[4-(methoxycarbonyl)-3-(trifluoromethanesulfonyloxy)phenyl]piperazine-1-carboxylate. To a stirred mixture of tert-butyl 4-(2,2-difluoroethyl)-2-(3-hydroxy-4-(methoxycarbonyl)phenyl)piperazine-1-carboxylate (2.0 g, 4.99 mmol) and 1,1,1-trifluoro-N-phenyl-N-(trifluoromethane)sulfonylmethanesulfonamide (2.61 g, 7.29 mmol) in DMF (15 mL) was added N,N-diisopropylethylamine (1.30 g, 10.04 mmol). The resulting mixture was stirred for additional 5 h at 70° C. The resulting mixture was quenched with water and extracted with ethyl acetate. The solvent was concentrated under reduced pressure. The residue was purified by silica gel column chromatography, eluted with petroleum ether/ethyl acetate (9:1) to afford the title compound (2.5 g, 94.00% yield) as a yellow oil. LCMS (ESI): [M+H]⁺=533.1.

Step 4: Bis(methyl 4-(4-(2,2-difluoroethyl)piperazin-2-yl)-2-(trifluoromethanesulfonyloxy)benzoate). To a stirred solution of bis(tert-butyl 4-(2,2-difluoroethyl)-2-(4-(methoxycarbonyl)-3-(trifluoromethanesulfonyloxy)phenyl)piperazine-1-carboxylate) (2.0 g, 1.87 mmol) in dichloromethane (2.5 mL) were added TFA (2.5 mL). The resulting mixture was stirred for additional 1.5 h at room temperature. The resulting mixture was concentrated under reduced pressure. The residue was purified by silica gel column chromatography, eluted with petroleum ether/ethyl acetate (3:1) to afford the title compound (1.4 g, 86.21% yield) as a yellow oil. LCMS (ESI): [M+H]⁺=433.1.

Step 5: tert-Butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate. To a stirred mixture of Bis(methyl 4-(4-(2,2-difluoroethyl)piperazin-2-yl)-2-(trifluoromethanesulfonyloxy)benzoate) (700 mg, 1.61 mmol) and tert-butyl 4-formyl-5-methoxy-7-methylindole-1-carboxylate (700 mg, 2.41 mmol) in dichloroethane (3 mL) were added Acetic acid (140 mg, 2.33 mmol) and Sodium triacetoxyborohydride (700 mg, 3.30 mmol) in portions. The resulting mixture was stirred for additional 6 h at 70° C. The solvent was removed under vacuum. The residue was purified by reverse flash chromatography with the following conditions: column, C18 silica gel; mobile phase, MeCN in Water (10 mmol/L NH4HCO₃), 500% to 100% gradient in 20 min; detector, UV 254 nm. to afford the title compound (500 mg, 43.76% yield) as a yellow solid. LCMS (ESI): [M+H]⁺=706.2.

Step 6: tert-Butyl 4-((4-(2,2-difluoroethyl)-2-(4-(methoxycarbonyl)-3-(methylamino)phenyl)piperazin-1-yl)methyl)-5-methoxy-7-methyl-1H-indole-1-carboxylate. To a stirred mixture of tert-Butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (300 mg, 0.42 mmol), Pd₂(dba)₃ (58 mg, 0.06 mmol), methylamine (90 mg, 2.89 mmol) Xantphos (59 mg, 0.10 mmol) and Cesium carbonate (420 mg, 1.28 mmol) in dioxane (5 mL) under nirtogen atmosphere. The resulting mixture was stirred for additional 2 h at 100° C. The solvent was removed under vacuum. The residue was purified by silica gel column chromatography, eluted with petroleum ether/ethyl acetate (7:1) to afford the title compound (200 mg, 80.19% yield) as a yellow solid. LCMS (ESI): [M+H]⁺=587.3.

Step 7: 4-(4-(2,2-Difluoroethyl)-1-((5-methoxy-7-methyl-1H-indol-4-yl)methyl)piperazin-2-yl)-2-(methylamino)benzoic acid. A mixture of tert-Butyl 4-((4-(2,2-difluoroethyl)-2-(4-(methoxycarbonyl)-3-(methylamino)phenyl)piperazin-1-yl)methyl)-5-methoxy-7-methyl-1H-indole-1-carboxylate (210 mg, 0.35 mmol) and Potassium hydroxide (200 mg, 3.56 mmol) in water (1 mL) and Ethanol (5 mL) was stirred for 2 h at 80° C. The reaction mixture was acidified to pH=6 with sat. citric acid solution. The solvent was removed under vacuum. The residue was purified by reverse flash chromatography with the following conditions: column, C18 silica gel; mobile phase, MeCN in Water (10 mmol/L NH4HCO₃), 10% to 30% gradient in 30 min; detector, UV 254 nm. to afford the title compound (67.6 mg, 39.97%) as a white solid. LCMS (ESI): [M+H]⁺=473.2. ¹H NMR (300 MHz, DMSO-d₆) δ 10.81 (s, 1H), 7.79 (d, J=8.1 Hz, 1H), 7.24 (d, J=2.7 Hz, 1H), 6.88 (s, 1H), 6.78 (d, J=7.9 Hz, 1H), 6.65 (s, 1H), 6.52 (s, 1H), 6.10 (tt, J=55.8, 4.3 Hz, 1H), 3.69 (s, 4H), 3.25 (s, 2H), 2.90 (s, 3H), 2.86-2.56 (m, 5H), 2.41 (s, 3H), 2.35-2.08 (m, 3H).

### Example 47. (S)-2-Chloro-4-(4-(2,2-difluoroethyl)-1-((5-methoxy-7-methyl-1H-indol-4-yl)methyl)piperazin-2-yl)benzoic acid & (R)-2-chloro-4-(4-(2,2-difluoroethyl)-1-((5-methoxy-7-methyl-1H-indol-4-yl)methyl)piperazin-2-yl)benzoic acid

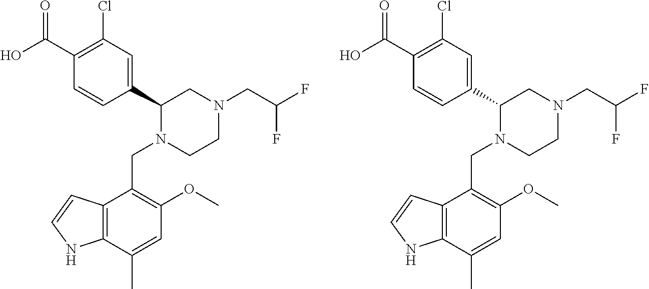 [SMILES: COc1cc(C)c2[nH]ccc2c1CN1CCN(CC(F)F)C[C@@H]1c1ccc(C(=O)O)c(Cl)c1 ; COc1cc(C)c2[nH]ccc2c1CN1CCN(CC(F)F)C[C@H]1c1ccc(C(=O)O)c(Cl)c1]

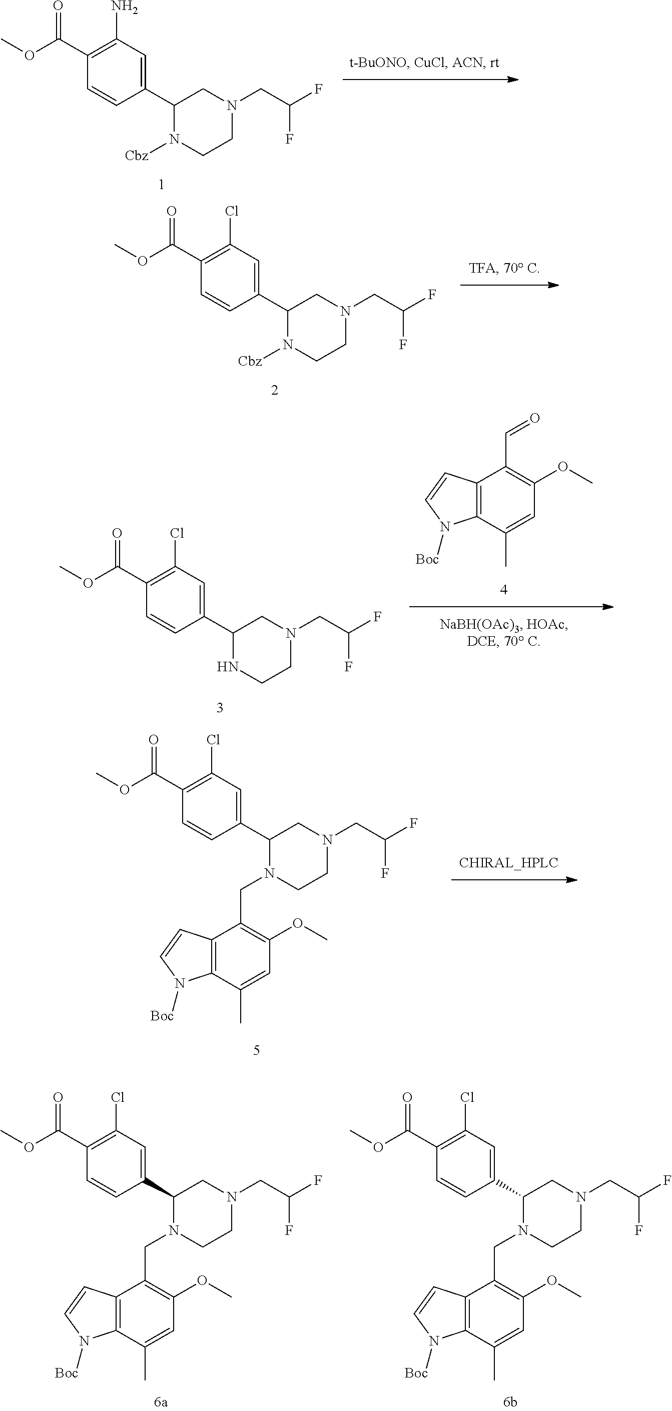

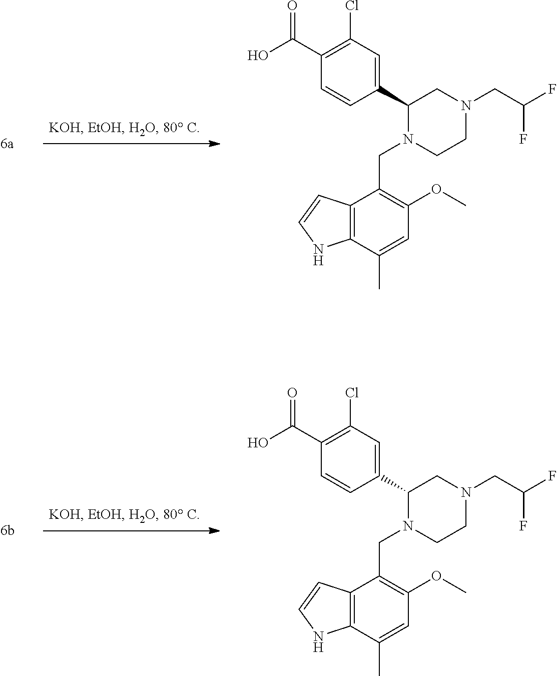 [SMILES: COc1cc(C)c2[nH]ccc2c1CN1CCN(CC(F)F)C[C@@H]1c1ccc(C(=O)O)c(Cl)c1 ; COc1cc(C)c2[nH]ccc2c1CN1CCN(CC(F)F)C[C@H]1c1ccc(C(=O)O)c(Cl)c1]

Step 1: Benzyl 2-(3-chloro-4-(methoxycarbonyl)phenyl)-4-(2,2-difluoroethyl)piperazine-1-carboxylate. To a stirred mixture of benzyl 2-(3-amino-4-(methoxycarbonyl)phenyl)-4-(2,2-difluoroethyl)piperazine-1-carboxylate (380 mg, 0.87 mmol) in ACN (5 mL) was added 3-methylbutyl nitrite (190 mg, 1.62 mmol) and CuCl (150 mg, 1.51 mmol) in portions at room temperature under nitrogen atmosphere. The resulting mixture was stirred for additional 1 h at room temperature. The reaction was quenched with water and extracted with ethyl acetate for 3 times. The combined organic layers was washed with brine, dried over anhydrous sodium sulfate. After filtration, the filtrate was concentrated under reduced pressure. The residue was purified by silica gel column chromatography, eluted with petroleum ether/ethyl acetate (6:1) to afford the title compound (280 mg, 70.52% yield) as a yellow oil. LCMS (ESI): [M+H]⁺=453.1.

Step 2: Methyl 2-chloro-4-(4-(2,2-difluoroethyl)piperazin-2-yl)benzoate. To a stirred solution of Benzyl 2-(3-chloro-4-(methoxycarbonyl)phenyl)-4-(2,2-difluoroethyl)piperazine-1-carboxylate (240 mg, 0.53 mmol) in TFA (1 mL) was stirred for 4 h at 70° C. The resulting mixture was concentrated under reduced pressure. The residue was purified by silica gel column chromatography, eluted with dichloromethane/methanol (9:1) to afford the title compound (165 mg, 97.68% yield) as a yellow oil. LCMS (ESI): [M+H]⁺=319.2.

Step 3: tert-Butyl 4-((2-(3-chloro-4-(methoxycarbonyl)phenyl)-4-(2,2-difluoroethyl)piperazin-1-yl)methyl)-5-methoxy-7-methyl-1H-indole-1-carboxylate. To a stirred mixture of Methyl 2-chloro-4-(4-(2,2-difluoroethyl)piperazin-2-yl)benzoate (200 mg, 0.62 mmol) and tert-butyl 4-formyl-5-methoxy-7-methylindole-1-carboxylate (280 mg, 0.96 mmol) in dichloroethane (4 mL) were added AcOH (39 mg, 0.64 mmol) and Sodium triacetoxyborohydride (260 mg, 1.22 mmol). The resulting mixture was stirred for additional 4 h at 70° C. The solvent was removed under vacuum. The residue was purified by silica gel column chromatography, eluted with petroleum ether/ethyl acetate (6:1) to afford the title compound (200 mg, 53.84% yield) as a yellow solid. LCMS (ESI): [M+H]⁺=592.2.

Step 4: tert-Butyl (S)-4-((2-(3-chloro-4-(methoxycarbonyl)phenyl)-4-(2,2-difluoroethyl)piperazin-1-yl)methyl)-5-methoxy-7-methyl-1H-indole-1-carboxylate (isomer 1, faster peak) & tert-Butyl (R)-4-((2-(3-chloro-4-(methoxycarbonyl)phenyl)-4-(2,2-difluoroethyl)piperazin-1-yl)methyl)-5-methoxy-7-methyl-1H-indole-1-carboxylate (isomer 2, slower peak).

tert-Butyl 4-((2-(3-chloro-4-(methoxycarbonyl)phenyl)-4-(2,2-difluoroethyl)piperazin-1-yl)methyl)-5-methoxy-7-methyl-1H-indole-1-carboxylate (170 mg, 0.294 mmol) was applied for further separation by Chiral HPLC with the folloing Column: CHIRALPAK IF, 2*25 cm, 5 μm; Mobile Phase A: Hex(0.5% 2M NH₃-MeOH)-HPLC, Mobile Phase B: Ethanol-HPLC; Flow rate: 20 mL/min; Gradient: 2% B to 2% B in 27 min; Wave Length: 220/254 nm; RT1(min): 14.488; RT2(min): 20.194; Sample Solvent: Ethanol-HPLC; Injection Volume: 0.7 mL; Number of Runs: 16.

tert-Butyl (S)-4-((2-(3-chloro-4-(methoxycarbonyl)phenyl)-4-(2,2-difluoroethyl)piperazin-1-yl)methyl)-5-methoxy-7-methyl-1H-indole-1-carboxylate (isomer 1, faster peak) (66 mg, 38.8% yield). LCMS (ESI): [M+H]⁺=592.2. t_{R}=2.18 min (CHIRALPAK IA-3, 4.6*50 mm, 3 μm, Hex(0.1% DEA):IPA=98:2, 1.0 mL/min, 254 nm).

tert-Butyl (R)-4-((2-(3-chloro-4-(methoxycarbonyl)phenyl)-4-(2,2-difluoroethyl)piperazin-1-yl)methyl)-5-methoxy-7-methyl-1H-indole-1-carboxylate (isomer 2, slower peak) (67 mg, 39.4% yield). LCMS (ESI): [M+H]⁺=592.2. t_{R}=3.00 min (CHIRALPAK IA-3, 4.6*50 mm, 3 μm, Hex(0.1% DEA):IPA=98:2, 1.0 mL/min, 254 nm).

Step 5: (S)-2-Chloro-4-(4-(2,2-difluoroethyl)-1-((5-methoxy-7-methyl-1H-indol-4-yl)methyl)piperazin-2-yl)benzoic acid & (R)-2-chloro-4-(4-(2,2-difluoroethyl)-1-((5-methoxy-7-methyl-1H-indol-4-yl)methyl)piperazin-2-yl)benzoic acid.

To a stirred mixture of tert-Butyl (S)-4-((2-(3-chloro-4-(methoxycarbonyl)phenyl)-4-(2,2-difluoroethyl)piperazin-1-yl)methyl)-5-methoxy-7-methyl-1H-indole-1-carboxylate (isomer 1, faster peak) (66 mg, 0.11 mmol) and Potassium hydroxide (136 mg, 2.424 mmol, 21.75 equiv) in water (0.2 mL) and Ethanol (2 mL). The resulting mixture was stirred for additional 2 h at 80° C. The reaction mixture was acidified to pH=6 with sat. citric acid solution. The solvent was removed under vacuum. The residue was purified by reverse flash chromatography with the following conditions: column, C18 silica gel; mobile phase, MeCN in Water (10 mmol/L NH4HCO₃), 5% to 30% gradient in 30 min; detector, UV 254 nm. to afford (S)-2-Chloro-4-(4-(2,2-difluoroethyl)-1-((5-methoxy-7-methyl-1H-indol-4-yl)methyl)piperazin-2-yl)benzoic acid (30.1 mg, 56.50% yield) as a white solid. LCMS (ESI): [M+H]⁺=478.2. ¹H NMR (300 MHz, DMSO-d₆) δ 13.36 (s, 1H), 10.84 (s, 1H), 7.86-7.67 (m, 2H), 7.55 (d, J=7.9 Hz, 1H), 7.26 (t, J=2.8 Hz, 1H), 6.66 (s, 1H), 6.42 (dd, J=3.1, 1.8 Hz, 1H), 6.10 (tt, J=55.7, 4.2 Hz, 1H), 3.73 (s, 3H), 3.60 (d, J=11.8 Hz, 1H), 3.41 (d, J=9.5 Hz, 1H), 3.20 (d, J=11.8 Hz, 1H), 2.91-2.57 (m, 5H), 2.41 (s, 3H), 2.32-2.10 (m, 3H).

To a stirred mixture of tert-Butyl (R)-4-((2-(3-chloro-4-(methoxycarbonyl)phenyl)-4-(2,2-difluoroethyl)piperazin-1-yl)methyl)-5-methoxy-7-methyl-1H-indole-1-carboxylate (isomer 2, slower peak) (67 mg, 0.113 mmol) and Potassium hydroxide (130 mg, 2.31 mmol) in water (0.2 mL) and Ethanol (2 mL). The resulting mixture was stirred for additional 2 h at 80° C. The reaction mixture was acidified to pH=6 with sat. citric acid solution. The solvent was removed under vacuum. The residue was purified by reverse flash chromatography with the following conditions: mobile phase, MeCN in Water (10 mmol/L NH4HCO₃), 5% to 30% gradient in 30 min; detector, UV 254 nm. to afford (R)-2-chloro-4-(4-(2,2-difluoroethyl)-1-((5-methoxy-7-methyl-1H-indol-4-yl)methyl)piperazin-2-yl)benzoic acid (31.0 mg, 57.32% yield) as a white solid. LCMS (ESI): [M+H]⁺=478.2. ¹H NMR (300 MHz, DMSO-d₆) δ 13.36 (s, 1H), 10.84 (s, 1H), 7.86-7.67 (m, 2H), 7.55 (d, J=7.9 Hz, 1H), 7.26 (t, J=2.8 Hz, 1H), 6.66 (s, 1H), 6.42 (dd, J=3.1, 1.8 Hz, 1H), 6.10 (tt, J=55.7, 4.2 Hz, 1H), 3.73 (s, 3H), 3.60 (d, J=11.8 Hz, 1H), 3.41 (d, J=9.5 Hz, 1H), 3.20 (d, J=11.8 Hz, 1H), 2.91-2.57 (m, 5H), 2.41 (s, 3H), 2.32-2.10 (m, 3H).

### Example 48. 4-(4-(2,2-Difluoroethyl)-1-((5-methoxy-7-methyl-1H-indol-4-yl)methyl)piperazin-2-yl)-2-(ethylamino)benzoic acid

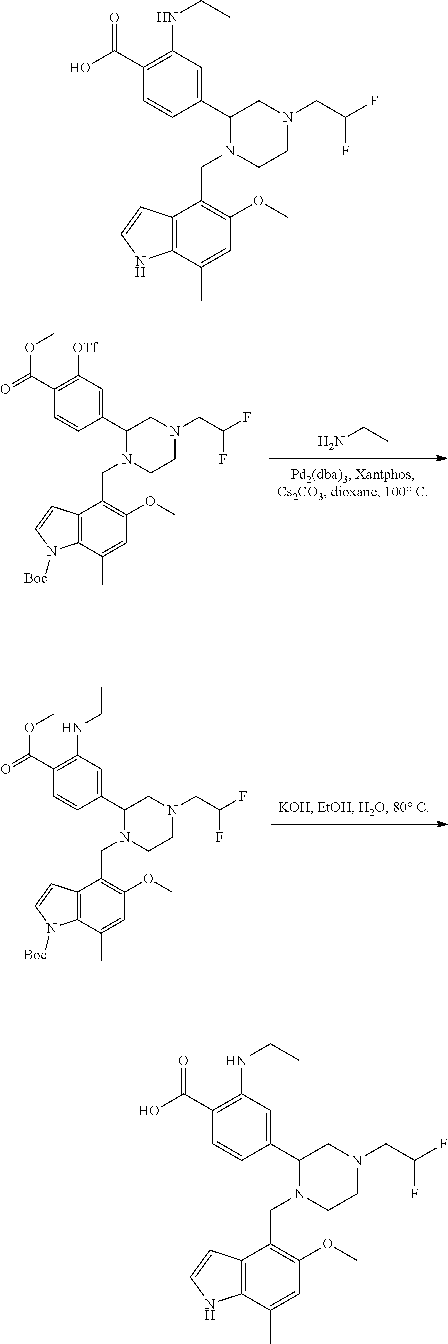

Step 1: tert-Butyl 4-((4-(2,2-difluoroethyl)-2-(3-(ethylamino)-4-(methoxycarbonyl)phenyl)piperazin-1-yl)methyl)-5-methoxy-7-methyl-1H-indole-1-carboxylate. To a stirred mixture of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (300 mg, 0.42 mmol), Pd₂(dba)₃ (59 mg, 0.06 mmol), ethylamine (120 mg, 2.66 mmol) Xantphos (59 mg, 0.10 mmol) and Cesium carbonate (420 mg, 1.28 mmol) in dioxane (5 mL) under nitrogen atmosphere. The resulting mixture was stirred for additional 2 h at 100° C. The solvent was removed. The residue was purified by silica gel column chromatography, eluted with petroleum ether/ethyl acetate (7:1) to afford the title compound (180 mg, 70.49% yield) as a yellow solid. LCMS (ESI): [M+H]⁺=601.3.

Step 2:4-(4-(2,2-Difluoroethyl)-1-((5-methoxy-7-methyl-1H-indol-4-yl)methyl)piperazin-2-yl)-2-(ethylamino)benzoic acid. To a stirred mixture of tert-Butyl 4-((4-(2,2-difluoroethyl)-2-(3-(ethylamino)-4-(methoxycarbonyl)phenyl)piperazin-1-yl)methyl)-5-methoxy-7-methyl-1H-indole-1-carboxylate (180 mg, 0.30 mmol) and Potassium hydroxide (336 mg, 5.98 mmol) in water (0.2 mL) and ethanol (2 mL). The resulting mixture was stirred for additional 2 h at 80° C. The reaction mixture was acidified to pH=6 with sat. citric acid solution. The solvent was removed under vacuum. The residue was purified by reverse flash chromatography with the following conditions: column, C18 silica gel; mobile phase, MeCN in Water (10 mmol/L NH4HCO₃), 5% to 30% gradient in 30 min; detector, UV 254 nm. to afford the title compound (40.8 mg, 27.98% yield) as a white solid. LCMS (ESI) [M+H]⁺=487.2. 1H NMR (300 MHz, DMSO-d₆) δ 10.81 (s, 1H), 7.80 (d, J=8.1 Hz, 1H), 7.23 (t, J=2.8 Hz, 1H), 6.86 (s, 1H), 6.74 (s, 1H), 6.64 (s, 1H), 6.52 (t, J=2.4 Hz, 1H), 6.10 (tt, J=55.7, 4.3 Hz, 1H), 3.69 (s, 4H), 3.29-3.14 (m, 4H), 2.90-2.54 (m, 5H), 2.41-2.04 (m, 6H), 1.22 (t, J=7.1 Hz, 3H).

### Example 49. (S)-2-Acetamido-4-(4-(2,2-difluoroethyl)-1-((5-methoxy-7-methyl-1H-indol-4-yl)methyl)piperazin-2-yl)benzoic acid & (R)-2-Acetamido-4-(4-(2,2-difluoroethyl)-1-((5-methoxy-7-methyl-1H-indol-4-yl)methyl)piperazin-2-yl)benzoic acid

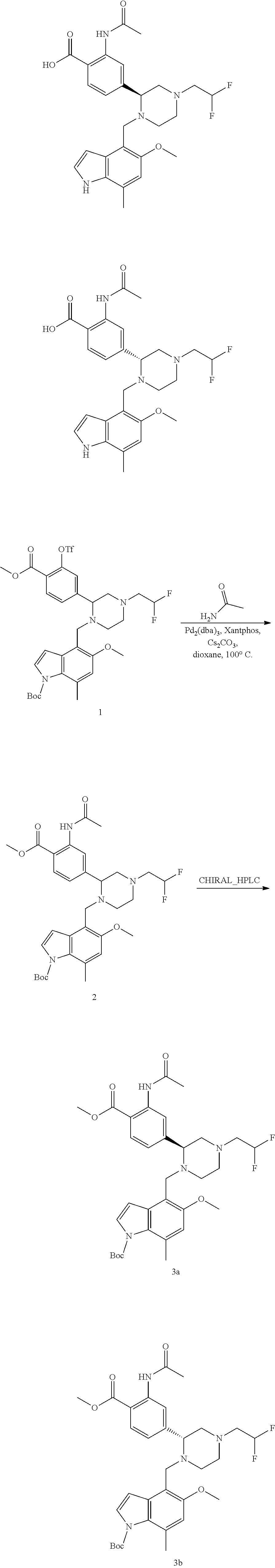

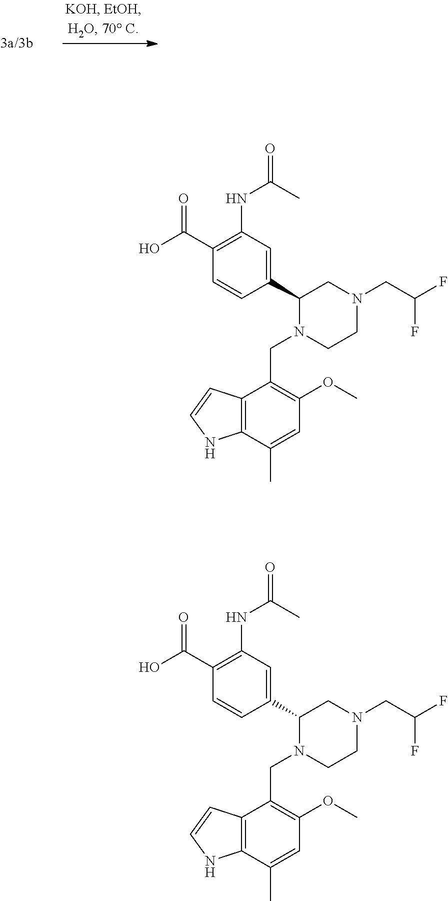 [SMILES: COc1cc(C)c2[nH]ccc2c1CN1CCN(CC(F)F)C[C@@H]1c1ccc(C(=O)O)c(NC(C)=O)c1 ; COc1cc(C)c2[nH]ccc2c1CN1CCN(CC(F)F)C[C@H]1c1ccc(C(=O)O)c(NC(C)=O)c1]

Step 1: tert-Butyl 4-((2-(3-acetamido-4-(methoxycarbonyl)phenyl)-4-(2,2-difluoroethyl)piperazin-1-yl)methyl)-5-methoxy-7-methyl-1H-indole-1-carboxylate. A mixture of tert-butyl 4-((4-(2,2-difluoroethyl)-2-(4-(methoxycarbonyl)-3-(((trifluoromethyl)sulfonyl)oxy)phenyl)piperazin-1-yl)methyl)-5-methoxy-7-methyl-1H-indole-1-carboxylate (380 mg, 0.53 mmol), acetamide (190 mg, 3.21 mmol), Pd₂(dba)₃ (74 mg, 0.08 mmol) Xantphos (75 mg, 0.13 mmol) and Cesium carbonate (530 mg, 1.62 mmol) in dioxane (5 mL) was stirred for 5 h at 100° C. under nitrogen atmosphere. The solvent was concentrated under reduced pressure. The residue was purified by silica gel column chromatography, eluted with petroleum ether/ethyl acetate (7:1) to afford the title compound (120 mg, 36.25% yield) as a yellow solid. LCMS (ESI): [M+H]⁺=615.3.

Step 2: tert-Butyl (S)-4-((2-(3-acctamido-4-(methoxycarbonyl)phenyl)-4-(2,2-difluoroethyl)piperazin-1-yl)methyl)-5-methoxy-7-methyl-1H-indole-1-carboxylate (isomer 1, faster peak) & tert-Butyl (R)-4-((2-(3-acetamido-4-(methoxycarbonyl)phenyl)-4-(2,2-difluoroethyl)piperazin-1-yl)methyl)-5-methoxy-7-methyl-1H-indole-1-carboxylate (isomer 2, slower peak).

tert-Butyl 4-((2-(3-acetamido-4-(methoxycarbonyl)phenyl)-4-(2,2-difluoroethyl)piperazin-1-yl)methyl)-5-methoxy-7-methyl-1H-indole-1-carboxylate (200 mg) was applied for further separation by Chiral HPLC with the folloing condition: Column: CHIRALPAK IE, 2*25 cm, 5 μm; Mobile Phase A: Hex(0.5% 2M NH₃-MeOH)-HPLC, Mobile Phase B: IPA-HPLC; Flow rate: 20 mL/min; Gradient: 10% B to 10% B in 33 min; Wave Length: 220/254 nm; RT1(min): 24.085; RT2(min): 29.055; Sample Solvent: Ethanol-HPLC; Injection Volume: 0.5 mL; Number of Runs: 17.

tert-Butyl (S)-4-((2-(3-acetamido-4-(methoxycarbonyl)phenyl)-4-(2,2-difluoroethyl)piperazin-1-yl)methyl)-5-methoxy-7-methyl-1H-indole-1-carboxylate (isomer 1, faster peak) (50 mg, 25.00% yield) LCMS (ESI): [M+H]⁺=615.3. tR=4.05 min (CHIRALPAK IA-3, 4.6*50 mm, 3 μm, Hex(0.1% DEA):IPA=90:10, 1.0 mL/min, 254 nm).

tert-Butyl (R)-4-((2-(3-acetamido-4-(methoxycarbonyl)phenyl)-4-(2,2-difluoroethyl)piperazin-1-yl)methyl)-5-methoxy-7-methyl-1H-indole-1-carboxylate (isomer 2, slower peak) (55 mg, 27.50% yield) LCMS (ESI): [M+H]⁺=615.3. tR=4.82 min (CHIRALPAK IA-3, 4.6*50 mm, 3 μm, Hex(0.1% DEA):IPA=90:10, 1.0 mL/min, 254 nm).

Step 3: (S)-2-Acetamido-4-(4-(2,2-difluoroethyl)-1-((5-methoxy-7-methyl-1H-indol-4-yl)methyl)piperazin-2-yl)benzoic acid & (R)-2-Acetamido-4-(4-(2,2-difluoroethyl)-1-((5-methoxy-7-methyl-1H-indol-4-yl)methyl)piperazin-2-yl)benzoic acid. A mixture of tert-Butyl (S)-4-((2-(3-acetamido-4-(methoxycarbonyl)phenyl)-4-(2,2-difluoroethyl)piperazin-1-yl)methyl)-5-methoxy-7-methyl-1H-indole-1-carboxylate (isomer 1, faster peak) (50 mg, 0.08 mmol) and Potassium hydroxide (92 mg, 1.64 mmol) in water (0.2 mL) and Ethanol (2 mL) was stirred for 2 h at 70° C. The reaction mixture was acidified to pH=6 with sat. citric acid solution. The solvent was removed under vacuum. The residue was purified by reverse flash chromatography with the following conditions: column, C18 silica gel; mobile phase, MeCN in Water (10 mmol/L NH4HCO₃), 50% to 30% gradient in 30 min; detector, UV 254 nm. to afford (S)-2-Acetamido-4-(4-(2,2-difluoroethyl)-1-((5-methoxy-7-methyl-1H-indol-4-yl)methyl)piperazin-2-yl)benzoic acid (30.1 mg, 73.93% yield) as a white solid. LCMS (ESI): [M+H]⁺=501.2. ¹H NMR (300 MHz, DMSO-d₆) δ 12.35 (s, 1H), 10.80 (s, 1H), 8.64 (s, 1H), 8.01 (d, J=8.1 Hz, 1H), 7.37-7.12 (m, 2H), 6.64 (s, 1H), 6.55-6.44 (m, 1H), 6.10 (tt, J=55.8, 4.3 Hz, 1H), 3.69 (s, 3H), 3.59 (d, J=11.9 Hz, 1H), 3.19 (brs, 2H), 2.89-2.54 (m, 5H), 2.40 (s, 3H), 2.34-2.15 (m, 3H), 2.11 (s, 3H).

A mixture of tert-Butyl (R)-4-((2-(3-acetamido-4-(methoxycarbonyl)phenyl)-4-(2,2-difluoroethyl)piperazin-1-yl)methyl)-5-methoxy-7-methyl-1H-indole-1-carboxylate (isomer 2, slower peak) (55 mg, 0.09 mmol) and Potassium hydroxide (100 mg, 1.78 mmol) in water (0.2 mL) and Ethanol (2 mL) was stirred for 2 h at 70° C. The reaction mixture was acidified to pH=6 with sat. citric acid solution. The solvent was removed under vacuum. The residue was purified by reverse flash chromatography with the following conditions: column, C18 silica gel; mobile phase, MeCN in Water (10 mmol/L NH4HCO₃), 5% to 30% gradient in 30 min; detector, UV 254 nm. to afford (R)-2-Acetamido-4-(4-(2,2-difluoroethyl)-1-((5-methoxy-7-methyl-1H-indol-4-yl)methyl)piperazin-2-yl)benzoic acid (36.6 mg, 81.72% yield) as a white solid. LCMS (ESI): [M+H]⁺=501.2. ¹H NMR (300 MHz, DMSO-d₆) δ 12.35 (s, 1H), 10.80 (s, 1H), 8.64 (s, 1H), 8.01 (d, J=8.1 Hz, 1H), 7.37-7.12 (m, 2H), 6.64 (s, 1H), 6.55-6.44 (m, 1H), 6.10 (tt, J=55.8, 4.3 Hz, 1H), 3.69 (s, 3H), 3.59 (d, J=11.9 Hz, 1H), 3.19 (brs, 2H), 2.89-2.54 (m, 5H), 2.40 (s, 3H), 2.34-2.15 (m, 3H), 2.11 (s, 3H).

### Example 50. 2-Hydroxy-4-((2S)-1-((5-methoxy-7-methyl-1H-indol-4-yl)methyl)-4-(3,3,3-trifluoropropyl)piperazin-2-yl)benzoic acid & 2-hydroxy-4-((2R)-1-((5-methoxy-7-methyl-1H-indol-4-yl)methyl)-4-(3,3,3-trifluoropropyl)piperazin-2-yl)benzoic acid

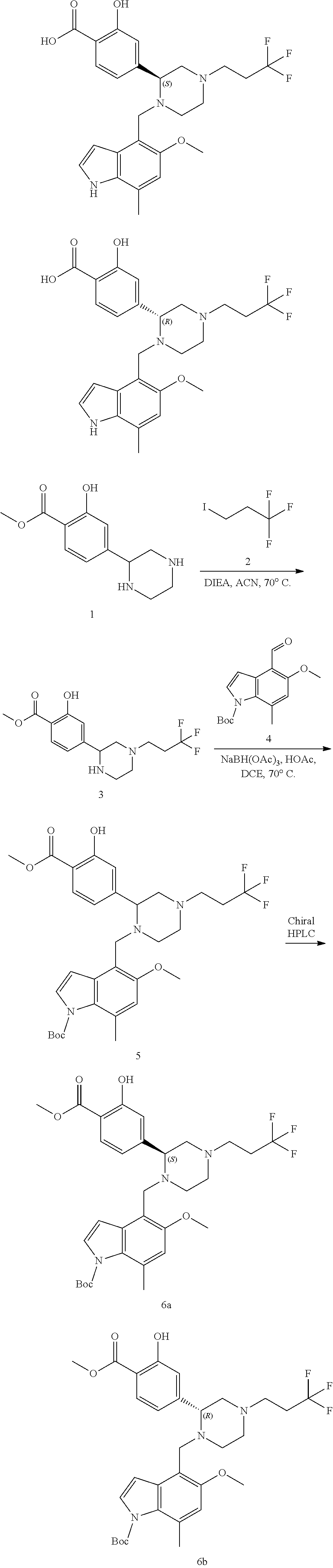

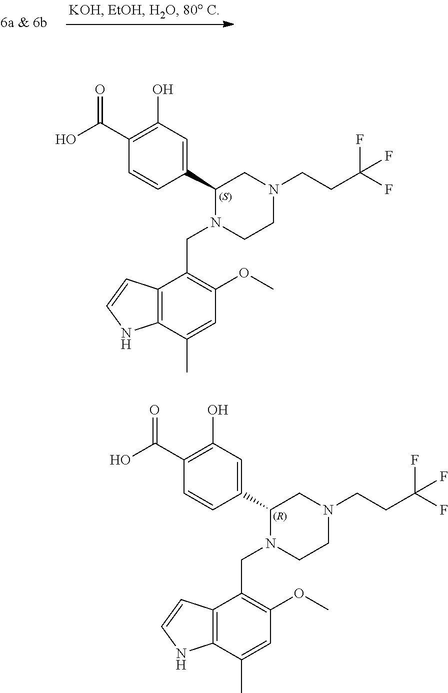

Step 1: Methyl 2-hydroxy-4-(4-(3,3,3-trifluoropropyl)piperazin-2-yl)benzoate. A solution of methyl 2-hydroxy-4-(piperazin-2-yl)benzoate (1.80 g, 7.62 mmol) in ACN (15 mL) was added 1,1,1-trifluoro-3-iodopropane (1.71 g, 7.62 mmol) and N,N-diisopropylethylamine (2.95 g, 22.85 mmol) at room temperature. The resulting mixture was stirred for overnight at 70° C. The resulting residue was purified by reverse phase chromatography (Acetonitrile 0-50%/0.1% Ammonium bicarbonate in water) to afford the title compound (760 mg, 30.0% yield) as a brown oil. LCMS (ESI): [M+H]⁺=332.9.

Step 2: tert-Butyl 4-((2-(3-hydroxy-4-(methoxycarbonyl)phenyl)-4-(3,3,3-trifluoropropyl)piperazin-1-yl)methyl)-5-methoxy-7-methylindole-1-carboxylate. A solution of methyl 2-hydroxy-4-(4-(3,3,3-trifluoropropyl)piperazin-2-yl)benzoate (730 mg, 2.20 mmol) in dichloroethane (3 mL) was added tert-butyl 4-formyl-5-methoxy-7-methylindole-1-carboxylate (763 mg, 2.64 mmol), NaBH(OAc)₃ (931 mg, 4.39 mmol) and HOAc (132 mg, 2.20 mmol) and stirred for 5 hours at 70° C. The solvent was concentrated under vacuum to afford the crude product. The resulting residue was purified by flash chromatography on silica gel eluting with petroleum ether/ethyl acetate (3/1) to afford the title compound (480 mg, 36.1% yield) as a yellow solid. LCMS (ESI): [M+H]⁺=606.4.

Step 3: tert-Butyl 4-(((2S)-2-(3-hydroxy-4-(methoxycarbonyl)phenyl)-4-(3,3,3-trifluoropropyl)piperazin-1-yl)methyl)-5-methoxy-7-methylindole-1-carboxylate (isomer 1, faster peak) & tert-butyl 4-(((2R)-2-(3-hydroxy-4-(methoxycarbonyl)phenyl)-4-(3,3,3-trifluoropropyl)piperazin-1-yl)methyl)-5-methoxy-7-methylindole-1-carboxylate (isomer 2, slower peak).

tert-Butyl 4-((2-(3-hydroxy-4-(methoxycarbonyl)phenyl)-4-(3,3,3-trifluoropropyl)piperazin-1-yl)methyl)-5-methoxy-7-methylindole-1-carboxylate (480 mg) was applied for further separation by Chiral HPLC with the following condition: Column: CHIRALPAK IA, 2*25 cm, 5 μm Mobile Phase A: Ethanol-HPLC, Mobile Phase B: Hex(0.5% 2M NH₃-MeOH)-HPLC; Flow rate: 20 mL/min; Gradient: 95% B to 95% B in 10 min; Wave Length: 220/254 nm; RT1(min): 4.54; RT2(min): 7.53; Sample Solvent: Ethanol-HPLC; Injection Volume: 0.5 mL; Number of Runs: 24.

tert-butyl 4-(((2S)-2-(3-Hydroxy-4-(methoxycarbonyl)phenyl)-4-(3,3,3-trifluoropropyl)piperazin-1-yl)methyl)-5-methoxy-7-methylindole-1-carboxylate (isomer 1, faster peak) (170 mg, 35.4% yield). LCMS (ESI): [M+H]⁺=606.2. t_{R}=0.972 min (CHIRALPAK A-3, 4.6*50 mm, 3 μm), Hex(0.1% DEA):Ethanol=85:15, 1.0 mL/min, 254 nm).

tert-butyl 4-(((2R)-2-(3-hydroxy-4-(methoxycarbonyl)phenyl)-4-(3,3,3-trifluoropropyl)piperazin-1-yl)methyl)-5-methoxy-7-methylindole-1-carboxylate (isomer 2, slower peak) (200 mg, 41.7% yield). LCMS (ESI): [M+H]⁺=606.2. t_{R}=1.336 min (CHIRALPAK IA-3, 4.6*50 mm, 3 μm), Hex(0.1% DEA):Ethanol=85:15, 1.0 mL/min, 254 nm).

Step 4: 2-Hydroxy-4-((2S)-1-((5-methoxy-7-methyl-1H-indol-4-yl)methyl)-4-(3,3,3-trifluoropropyl)piperazin-2-yl)benzoic acid & 2-hydroxy-4-((2R)-1-((5-methoxy-7-methyl-1H-indol-4-yl)methyl)-4-(3,3,3-trifluoropropyl)piperazin-2-yl)benzoic acid. A solution of tert-butyl 4-(((2S)-2-(3-hydroxy-4-(methoxycarbonyl)phenyl)-4-(3,3,3-trifluoropropyl)piperazin-1-yl)methyl)-5-methoxy-7-methylindole-1-carboxylate (170 mg, 0.28 mmol) in Ethanol (2 mL) was added Potassium hydroxide (156 mg, 2.80 mmol) in water (0.5 mL) and stirred for 1.5 hours at 80° C. The mixture was allowed to cool down to room temperature and was neutralized to pH=7 with Citric acid. The resulting residue was purified by reverse phase chromatography (Acetonitrile 0-40%/0.1% Ammonium bicarbonate in water) to afford 2-hydroxy-4-((2S)-1-((5-methoxy-7-methyl-1H-indol-4-yl)methyl)-4-(3,3,3-trifluoropropyl)piperazin-2-yl)benzoic acid (83.6 mg, 59.0% yield) as a white solid. LCMS (ESI): [M+H]⁺=492.1. ¹H NMR (300 MHz, DMSO-d₆) δ 10.88 (s, 1H), 7.74 (d, J=7.8 Hz, 1H), 7.28 (s, 1H), 7.01-6.91 (m, 2H), 6.70 (s, 1H), 6.47 (s, 1H), 3.75-3.65 (m, 5H), 3.25-3.05 (m, 2H), 2.85-2.75 (m, 3H), 2.65-2.55 (m, 6H), 2.21-1.95 (m, 3H).

A solution of tert-butyl 4-(((2S)-2-(3-hydroxy-4-(methoxycarbonyl)phenyl)-4-(3,3,3-trifluoropropyl)piperazin-1-yl)methyl)-5-methoxy-7-methylindole-1-carboxylate (200 mg, 0.33 mmol) in Ethanol (3 mL) was added Potassium hydroxide (185 mg, 3.30 mmol) in water (0.6 mL) for 1.5 hours at 80° C. The mixture was allowed to cool down to room temperature and was neutralized to pH 7 with Citric acid. The resulting residue was purified by reverse phase chromatography (Acetonitrile 0-40%/0.1% Ammonium bicarbonate in water) to afford 2-hydroxy-4-((2R)-1-((5-methoxy-7-methyl-1H-indol-4-yl)methyl)-4-(3,3,3-trifluoropropyl)piperazin-2-yl)benzoic acid (77.9 mg, 45.9% yield) as a white solid. LCMS (ESI): [M+H]⁺=492.1. ¹H NMR (300 MHz, DMSO-d₆) δ 10.88 (s, 1H), 7.74 (d, J=7.8 Hz, 1H), 7.28 (s, 1H), 7.01-6.91 (m, 2H), 6.70 (s, 1H), 6.47 (s, 1H), 3.75-3.65 (m, 5H), 3.25-3.05 (m, 2H), 2.85-2.75 (m, 3H), 2.65-2.55 (m, 6H), 2.21-1.95 (m, 3H).

### Example 50. 2-Amino-4-(4-(2,2-difluoroethyl)-1-((5-methoxy-7-methyl-1H-indol-4-yl)methyl)piperazin-2-yl)benzoic acid

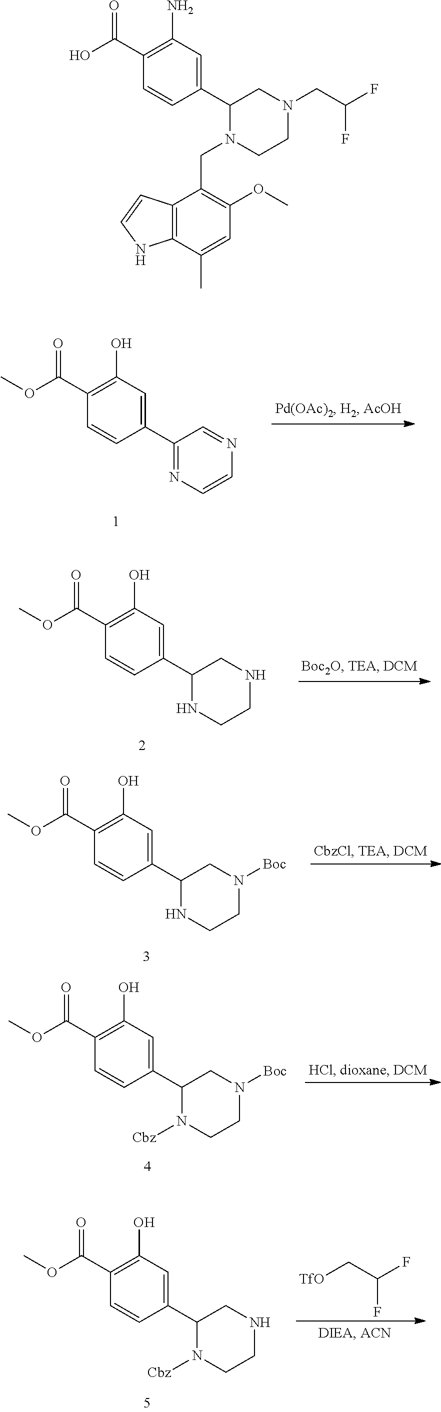

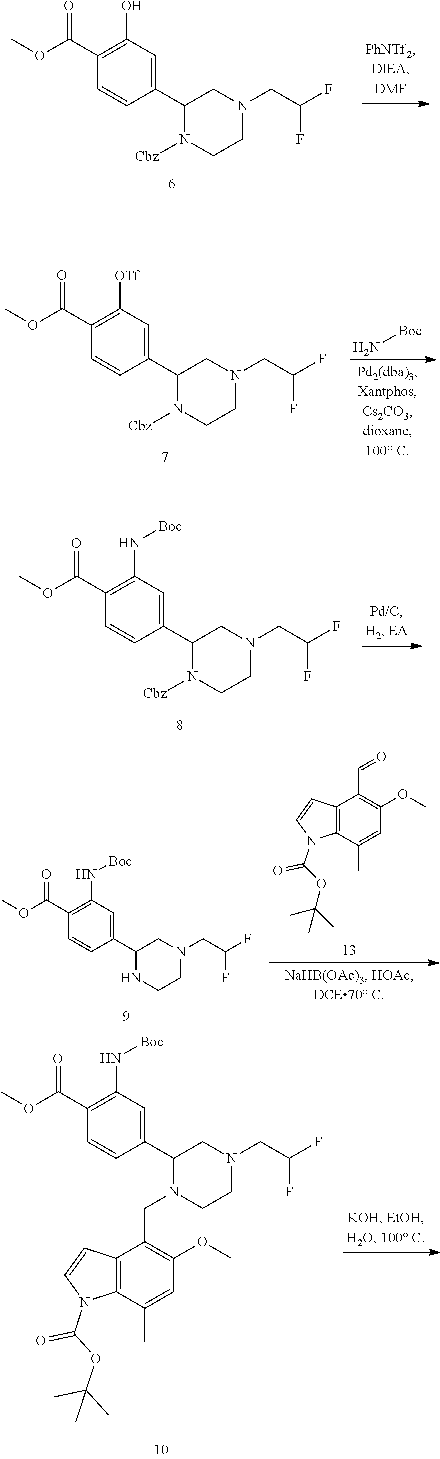

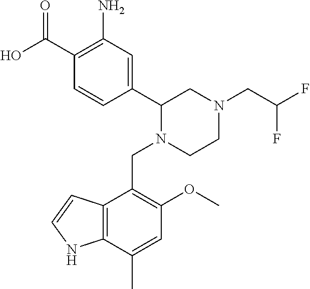 [SMILES: COc1cc(C)c2[nH]ccc2c1CN1CCN(CC(F)F)CC1c1ccc(C(=O)O)c(N)c1]

Step 1: Methyl 2-hydroxy-4-(piperazin-2-yl)benzoate. A solution of methyl 2-hydroxy-4-(pyrazin-2-yl)benzoate (4 g, 17.37 mmol) in AcOH (20 mL) was added Pd(OAc)₂ (1.9 g, 8.46 mmol). The resulting mixture was stirred for additional 1 overnight at room temperature under hydrogen. The resulting mixture was filtered. The filtrate was concentrated under reduced pressure to afford the title compound (5 g) as a yellow solid and as crude. LCMS (ESI): [M+H]⁺=237.1.

Step 2: tert-Butyl 3-(3-hydroxy-4-(methoxycarbonyl)phenyl)piperazine-1-carboxylate. To a stirred solution of methyl 2-hydroxy-4-(piperazin-2-yl)benzoate (5 g, 21.16 mmol) and Boc₂O (4.62 g, 21.16 mmol) in DCM (40 mL) were added Triethylamine (6.25 g, 61.79 mmol) dropwise at room temperature. The resulting mixture was stirred for additional 1 h at room temperature. The reaction was quenched with Water. The resulting mixture was extracted with ethyl acetate. The combined organic layers were dried over anhydrous sodium sulfate. After filtration, the filtrate was concentrated under reduced pressure. The residue was purified by silica gel column chromatography, eluted with petroleum ether/ethyl acetate (3:2) to afford the title compound (5.2 g, 73.05% yield) as a yellow oil. LCMS (ESI): [M+H]⁺=337.1.

Step 3:1-Benzyl 4-(tert-butyl)₂-(3-hydroxy-4-(methoxycarbonyl)phenyl)piperazine-1,4-dicarboxylate. To a stirred solution of tert-Butyl 3-(3-hydroxy-4-(methoxycarbonyl)phenyl)piperazine-1-carboxylate (5 g, 3.71 mmol) and Triethylamine (1248 mg, 12.33 mmol) in DCM (30 mL) was added CbzCl (748 mg, 4.38 mmol) at room temperature. The resulting mixture was stirred for additional 1 h at room temperature. The reaction was quenched with water and extracted with ethyl acetate. The solvent was concentrated under reduced pressure. The residue was purified by silica gel column chromatography, eluted with petroleum ether/ethyl acetate (3:2) to afford the title compound (5.5 g, 78.64% yield) as a yellow oil. LCMS (ESI): [M+H]⁺=471.2.

Step 4: Benzyl 2-(3-hydroxy-4-(methoxycarbonyl)phenyl)piperazine-1-carboxylate. A solution of 1-Benzyl 4-(tert-butyl)₂-(3-hydroxy-4-(methoxycarbonyl)phenyl)piperazine-1,4-dicarboxylate (5.5 g, 11.68 mmol) in 4 M HCl in dioxane (20 mL) was stirred for additional 3 h at room temperature. The resulting mixture was concentrated under reduced pressure. to afford then title compound (3.7 g, 85.46% yield) as a yellow oil. The crude product was used in the next step directly without further purification. LCMS (ESI): [M+H]⁺=371.2.

Step 5: Benzyl 4-(2,2-difluoroethyl)-2-[3-hydroxy-4-(methoxycarbonyl)phenyl]piperazine-1-carboxylate. To a stirred mixture of Benzyl 2-(3-hydroxy-4-(methoxycarbonyl)phenyl)piperazine-1-carboxylate (3.5 g, 9.44 mmol) and 2,2-difluoroethyl trifluoromethanesulfonate (2.27 g, 10.58 mmol) in ACN (30 mL) was added N,N-diisopropylethylamine (3.51 g, 27.11 mmol) dropwise at room temperature. The resulting mixture was stirred for additional 1 h at room temperature. The resulting mixture was concentrated under reduced pressure. The residue was purified by silica gel column chromatography, eluted with petroleum ether/ethyl acetate (5:1) to afford the title compound (3.5 g, 85.26% yield) as a yellow oil. LCMS (ESI): [M+H]⁺=435.2.

Step 6: Benzyl 4-(2,2-difluoroethyl)-2-(4-(methoxycarbonyl)-3-(((trifluoromethyl)sulfonyl)oxy)phenyl)piperazine-1-carboxylate. To a stirred mixture of Benzyl 4-(2,2-difluoroethyl)-2-[3-hydroxy-4-(methoxycarbonyl)phenyl]piperazine-1-carboxylate (3.5 g, 8.05 mmol) and 1,1,1-trifluoro-N-phenyl-N-((trifluoromethyl)sulfonyl)methanesulfonamide (3.51 g, 9.82 mmol) in DMF (20 mL) were added N,N-diisopropylethylamine (3.16 g, 24.41 mmol) dropwise at room temperature. The resulting mixture was stirred for additional 2 h at room temperature. The reaction was quenched by the addition of water. The resulting mixture was extracted with ethyl acetate. The combined organic layers were washed with water, dried over anhydroussodium sulfate. After filtration, the filtrate was concentrated under reduced pressure. The residue was purified by silica gel column chromatography, eluted with petroleum ether/ethyl acetate (5:1) to afford the title compound (3.5 g, 76.69% yield) as a yellow oil. LCMS (ESI): [M+H]⁺=567.1.

Step 7: Benzyl 2-(3-((tert-butoxycarbonyl)amino)-4-(methoxycarbonyl)phenyl)-4-(2,2-difluoroethyl)piperazine-1-carboxylate. A solution of Benzyl 4-(2,2-difluoroethyl)-2-(4-(methoxycarbonyl)-3-(((trifluoromethyl)sulfonyl)oxy)phenyl)piperazine-1-carboxylate (1 g, 1.76 mmol) and tert-butyl carbamate (300 mg, 2.56 mmol) in dioxane (10 mL) was added Xantphos (205 mg, 0.35 mmol), Cesium carbonate (1.7 g, 5.21 mmol) and Pd₂(dba)₃ (242 mg, 0.26 mmol) and stirred for 2 h at 100° C. under nitrogen. The solvent was removed under reduced pressure. The residue was purified by silica gel column chromatography, eluted with petroleum ether/ethyl acetate (5:1) to afford the title cpompound (800 mg, 84.93% yield) as a yellow solid. LCMS (ESI): [M+H]⁺=534.2.

Step 8: Methyl 2-((tert-butoxycarbonyl)amino)-4-(4-(2,2-difluoroethyl)piperazin-2-yl)benzoate. A mixture of Benzyl 2-(3-((tert-butoxycarbonyl)amino)-4-(methoxycarbonyl)phenyl)-4-(2,2-difluoroethyl)piperazine-1-carboxylate (1 g, 1.87 mmol) and 10% Pd/C (0.40 g) in ethyl acetate (10 mL) was stirred for overnight at room temperature under hydrogen atmosphere. The resulting mixture was filtered, the filtrate was concentrated under reduced pressure to afford the title compound (650 mg, 86.83% yield) as a yellow solid. LCMS (ESI): [M+H]⁺=400.2.

Step 9: tert-Butyl 4-((2-(3-((tert-butoxycarbonyl)amino)-4-(methoxycarbonyl)phenyl)-4-(2,2-difluoroethyl)piperazin-1-yl)methyl)-5-methoxy-7-methyl-1H-indole-1-carboxylate. To a stirred mixture of Methyl 2-((tert-butoxycarbonyl)amino)-4-(4-(2,2-difluoroethyl)piperazin-2-yl)benzoate (350 mg, 0.87 mmol) and tert-butyl 4-formyl-5-methoxy-7-methylindole-1-carboxylate (500 mg, 1.72 mmol) in dichloroethane (10 mL) was added AcOH (60 mg, 0.87 mmol). The resulting mixture was stirred for additional 1 h at 50° C. To the above mixture was added Sodium triacetoxyborohyride (350 mg, 1.65 mmol). The resulting mixture was stirred for additional 7 h at 70° C. The solvent was removed under vacuum. The residue was purified by silica gel column chromatography, eluted with petroleum ether/ethyl acetate (5:1) to afford the title compound (350 mg, 59.37% yield) as a yellow solid. LCMS (ESI): [M+H]⁺=673.3.

Step 10: 2-Amino-4-(4-(2,2-difluoroethyl)-1-((5-methoxy-7-methyl-1H-indol-4-yl)methyl)piperazin-2-yl)benzoic acid. A mixture of tert-Butyl 4-((2-(3-((tert-butoxycarbonyl)amino)-4-(methoxycarbonyl)phenyl)-4-(2,2-difluoroethyl)piperazin-1-yl)methyl)-5-methoxy-7-methyl-1H-indole-1-carboxylate (300 mg, 0.51 mmol) and Potassium hydroxide (300 mg, 5.34 mmol) in ethanol (5 mL) and water (1 mL) was stirred for 24 h at 100° C. The resulting mixture was concentrated under reduced pressure. The residue was purified by Prep-HPLC to afford the title compound (150 mg, 73.3% yield) as a pink solid. LCMS (ESI): [M+H]⁺=459.2, ¹H NMR (400 MHz, DMSO-d₆) δ 10.82 (s, 1H), 7.74 (d, J=8.1 Hz, 1H), 7.24 (t, J=2.8 Hz, 1H), 6.98-6.56 (m, 3H), 6.54-6.41 (m, 1H), 6.29-5.89 (m, 1H), 3.72 (s, 4H), 3.27-3.04 (m, 2H), 2.86-2.54 (m, 5H), 2.42 (s, 3H), 2.30-2.03 (m, 3H).

### Example 51. (R)-2-Amino-4-(4-(2,2-difluoroethyl)-1-((5-methoxy-7-methyl-1H-indol-4-yl)methyl)piperazin-2-yl)benzoic acid

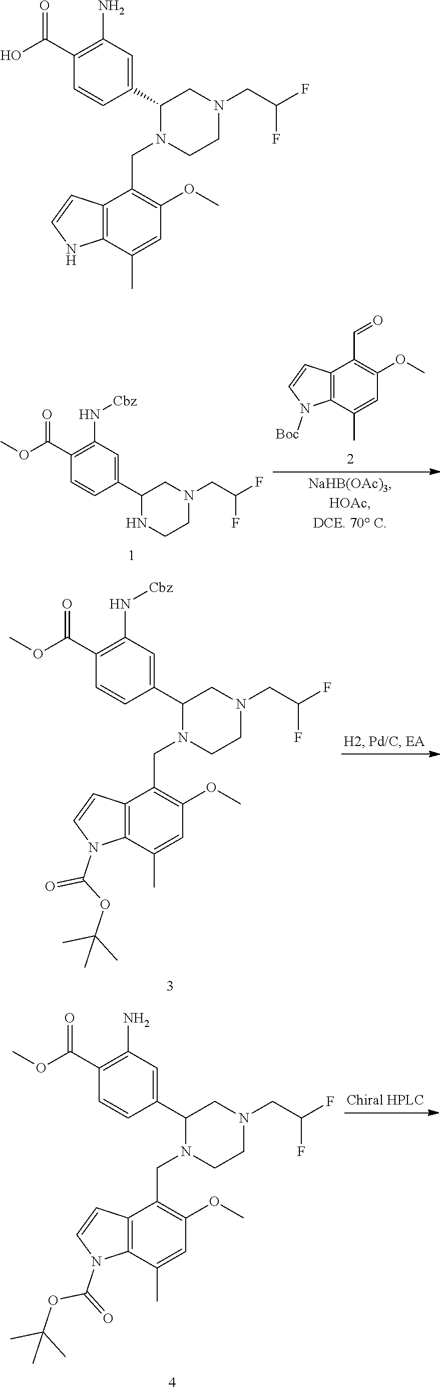 [SMILES: COc1cc(C)c2[nH]ccc2c1CN1CCN(CC(F)F)C[C@H]1c1ccc(C(=O)O)c(N)c1 ; COC(=O)c1ccc(C2CN(CC(F)F)CCN2)cc1NC(=O)OCc1ccccc1 ; COc1cc(C)c2c(ccn2C(=O)OC(C)(C)C)c1C=O ; COC(=O)c1ccc(C2CN(CC(F)F)CCN2Cc2c(OC)cc(C)c3c2ccn3C(=O)OC(C)(C)C)cc1NC(=O)OCc1ccccc1 ; COC(=O)c1ccc(C2CN(CC(F)F)CCN2Cc2c(OC)cc(C)c3c2ccn3C(=O)OC(C)(C)C)cc1N]

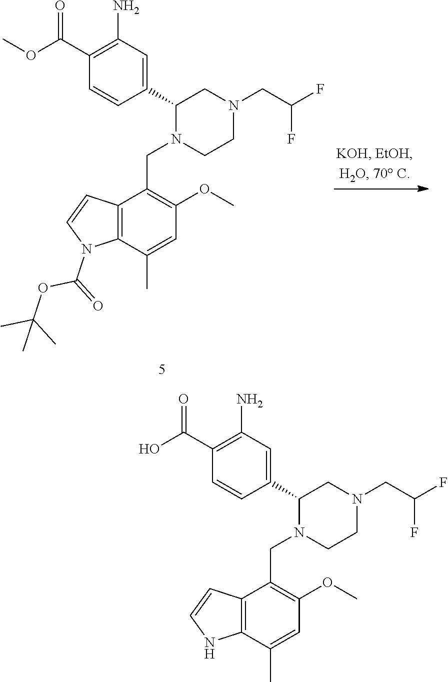 [SMILES: COC(=O)c1ccc([C@@H]2CN(CC(F)F)CCN2Cc2c(OC)cc(C)c3c2ccn3C(=O)OC(C)(C)C)cc1N ; COc1cc(C)c2[nH]ccc2c1CN1CCN(CC(F)F)C[C@H]1c1ccc(C(=O)O)c(N)c1]

Step 1: tert-Butyl 4-((2-(3-(((benzyloxy)carbonyl)amino)-4-(methoxycarbonyl)phenyl)-4-(2,2-difluoroethyl)piperazin-1-yl)methyl)-5-methoxy-7-methyl-1H-indole-1-carboxylate. A mixture of methyl 2-(((benzyloxy)carbonyl)amino)-4-(4-(2,2-difluoroethyl)piperazin-2-yl)benzoate (2 g, 4.61 mmol), tert-butyl 4-formyl-5-methoxy-7-methylindole-1-carboxylate (2.00 g, 6.92 mmol), HOAc (399.00 mg, 6.64 mmol) and NaBH(OAc)₃ (1.96 g, 9.22 mmol) in dichloroethane (20 mL) was stirred for 24 h at 70° C. The solvent was removed under vacuum. The residue was purified by reversed-phase flash chromatography with the following conditions: column, C18 silica gel; mobile phase, MeCN in Water (10 mmol/L NH4HCO₃), 10% to 50% gradient in 10 min; detector, UV 254 nm. This resulted in the title compound (2.7 g, 82.79% yield) as a yellow solid. LCMS (ESI): [M+H]⁺=707.7

Step 2: tert-Butyl 4-((2-(3-amino-4-(methoxycarbonyl)phenyl)-4-(2,2-difluoroethyl)piperazin-1-yl)methyl)-5-methoxy-7-methyl-1H-indole-1-carboxylate. A mixture of tert-Butyl 4-((2-(3-(((benzyloxy)carbonyl)amino)-4-(methoxycarbonyl)phenyl)-4-(2,2-difluoroethyl)piperazin-1-yl)methyl)-5-methoxy-7-methyl-1H-indole-1-carboxylate (2.7 g, 3.8 mmol) and 10% Pd/C (1.35 g) in ethyl acetate (200 mL) was stirred for 12 h at room temperature. The solids were filtered out. The filtrate was concentrated to afford the title comound (1.9 g, 86.8% yield) as a white solid. LCMS (ESI): [M+H]⁺=573.3.

Step 3: tert-Butyl (R)-4-((2-(3-amino-4-(methoxycarbonyl)phenyl)-4-(2,2-difluoroethyl)piperazin-1-yl)methyl)-5-methoxy-7-methyl-1H-indole-1-carboxylate (isomer 2, slower peak). tert-Butyl 4-((2-(3-amino-4-(methoxycarbonyl)phenyl)-4-(2,2-difluoroethyl)piperazin-1-yl)methyl)-5-methoxy-7-methyl-1H-indole-1-carboxylate was applied for further separation by Chiral HPLC with the following condition: Column: CHIRALCEL OD-3, 3.0*100 mm, 3 um; Mobile Phase A: CO₂, Mobile Phase B: IPA; Flow rate: 100 mL/min; Gradient: isocratic 20% B; Column Temperature(° C.): 35; Back Pressure(bar): 100; Wave Length: 220 nm; RT1(min): 7.75; RT2(min): 9.22; Sample Solvent: MEOH; Injection Volume: 1.5 mL; Number Of Runs: 35.

tert-Butyl (R)-4-((2-(3-amino-4-(methoxycarbonyl)phenyl)-4-(2,2-difluoroethyl)piperazin-1-yl)methyl)-5-methoxy-7-methyl-1H-indole-1-carboxylate (isomer 2, slower peak) (550 mg, 28.9% yield) as white solid. LCMS (ESI): [M+H]⁺=573.3. tR=2.22 min (CHIRALPAK IE-3, 4.6*50 mm, 3 um, Hex:DCM=3:1)(0.1% DEA):Ethanol=80:20, 1.0 mL/min, 254 nm).

Step 4: (R)-2-Amino-4-(4-(2,2-difluoroethyl)-1-((5-methoxy-7-methyl-1H-indol-4-yl)methyl)piperazin-2-yl)benzoic acid. To a stirred solution of tert-Butyl (R)-4-((2-(3-amino-4-(methoxycarbonyl)phenyl)-4-(2,2-difluoroethyl)piperazin-1-yl)methyl)-5-methoxy-7-methyl-1H-indole-1-carboxylate (550 mg, 0.96 mmol) and Potassium hydroxide (300 mg, 5.34 mmol) in Ethanol (4 mL) and water (0.5 mL) was stirred for 12 h at 70° C. The mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH4HCO₃), 10% to 50% gradient in 10 min; detector, UV 254 nm. This resulted in the title compound (339.4 mg, 77.0% yield) as a white solid. LCMS (ESI): [M+H]⁺=459.5, ¹H NMR (400 MHz, DMSO-d₆) δ 10.83 (s, 1H), 8.54 (s, 1H), 7.72 (d, J=8.2 Hz, 1H), 7.25 (t, J=2.8 Hz, 1H), 6.93 (s, 1H), 6.75 (s, 1H), 6.67 (s, 1H), 6.48 (t, J=3.1, 1.9 Hz, 1H), 6.12 (t, J=55.7, 4.3 Hz, 1H), 3.73 (s, 3H), 3.65 (d, J=11.8 Hz, 1H), 3.26-3.11 (m, 2H), 2.67 (s, 4H), 2.58 (s, 1H), 2.43 (s, 3H), 2.26 (t, J=10.7 Hz, 1H), 2.16 (d, J=8.1 Hz, 2H).

### Example 52. 4-(6-(Difluoromethyl)-2-((5-methoxy-7-methyl-1H-indol-4-yl)methyl)-hexahydro-1H-pyrrolo(1,2-a)pyrazin-3-yl)benzoic acid

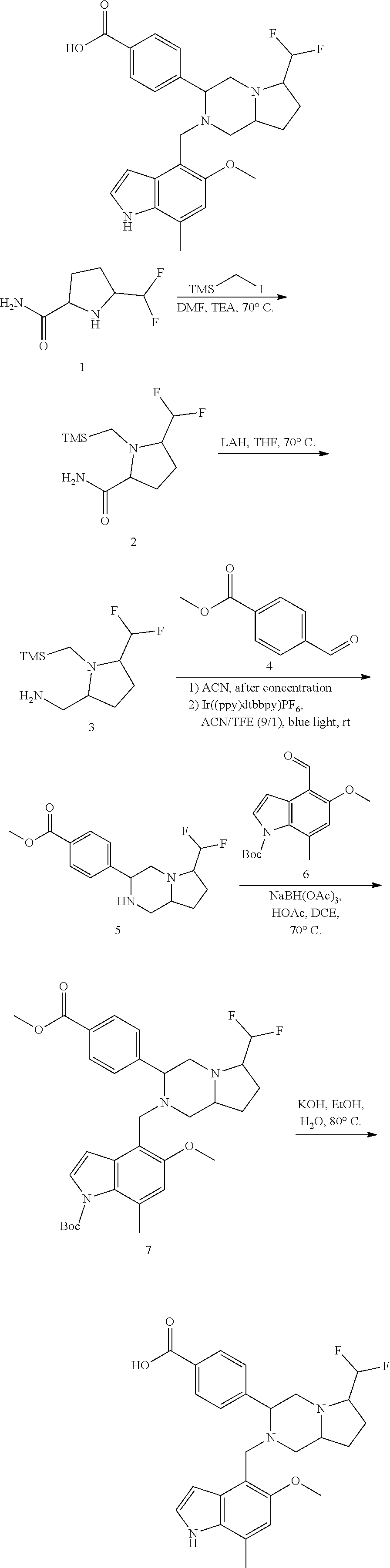

Step 1:5-(Difluoromethyl)-1-((trimethylsilyl)methyl)pyrrolidine-2-carboxamide. A solution of 5-(difluoromethyl)pyrrolidine-2-carboxamide (500 mg, 3.04 mmol), (iodomethyl)trimethylsilane (2.3 g, 10.66 mmol) and Triethylamine (1.5 g, 15.22 mmol) in DMF (4 mL) was stirred for 24 h at 70° C. The crude product was purified by reverse phase flash with the following conditions (acetonitrile, 5-43%/10 mM NH₄HCO₃ in water) to afford the title compound (233 mg, 30.55% yield) as a light yellow oil. LCMS (ESI): [M+H]⁺=251.1.

Step 2: 1-(5-(Difluoromethyl)-1-((trimethylsilyl)methyl)pyrrolidin-2-yl)methanamine. A solution of 5-(difluoromethyl)-1-((trimethylsilyl)methyl)pyrrolidine-2-carboxamide (176 mg, 0.70 mmol) in THF (15 mL) was added Lithium Aluminum Hydride (132 mg, 3.48 mmol) at 0° C. and stirred for 30 min at 70° C. The reaction was quenched with water (0.15 mL), NaOH (15% wt in water, 0.15 mL) and water (0.45 mL) at 0° C. The resulting mixture was filtered, the filter cake was washed with THF. The filtrate was concentrated under reduced pressure to afford the title compound (165 mg, 99.30% yield) as a light yellow liquid. LCMS (ESI): [M+H]⁺=237.2.

Step 3: Methyl 4-(6-(difluoromethyl)-octahydropyrrolo(1,2-a)pyrazin-3-yl)benzoate. A solution of 1-(5-(difluoromethyl)-1-((trimethylsilyl)methyl)pyrrolidin-2-yl)methanamine (165 mg, 0.70 mmol) and methyl 4-formylbenzoate (114 mg, 0.70 mmol) in acetonitrile (0.9 mL) was stirred for 3 h at room temperature under nitrogen atmosphere. To the above mixture was added (Ir(dtbbpy)(ppy)₂)(PF₆) (63 mg, 0.07 mmol) and Difluoroethanol (0.1 mL) in portions at room temperature. The resulting mixture was stirred in blue LED for additional 4 h at room temperature under nitrogen atmosphere. The crude product was purified by reverse phase flash with the following conditions (acetonitrile, 5-60%/10 mM NH₄HCO₃ in water) to afford the title compound (23 mg, 10.62% yield) as a light yellow solid. LCMS (ESI): [M+H]⁺=311.2.

Step 4: tert-Butyl 4-((6-(difluoromethyl)-3-(4-(methoxycarbonyl)phenyl)-hexahydro-1H-pyrrolo(1,2-a)pyrazin-2-yl)methyl)-5-methoxy-7-methylindole-1-carboxylate. 1

A solution of methyl 4-(6-(difluoromethyl)-octahydropyrrolo(1,2-a)pyrazin-3-yl)benzoate (23 mg, 0.07 mmol), tert-butyl 4-formyl-5-methoxy-7-methylindole-1-carboxylate (26 mg, 0.09 mmol), HOAc (13 mg, 0.22 mmol) and NaBH(OAc)₃ (31 mg, 0.15 mmol) in 1,2-dichloroethane (0.5 mL) was stirred for 3 h at 70° C. The resulting mixture was extracted with ethyl acetate. The combined organic layers were washed with brine, dried over anhydrous Na₂SO₄. After filtration, the filtrate was concentrated under reduced pressure. The residue was purified by silica gel column chromatography, eluted with petroleum ether/ethyl acetate (1:1) to afford the title compound (10 mg, 23.12% yield) as a white solid. LCMS (ESI): [M+H]⁺=584.3.

Step 5: 4-(6-(Difluoromethyl)-2-((5-methoxy-7-methyl-1H-indol-4-yl)methyl)-hexahydro-1H-pyrrolo(1,2-a)pyrazin-3-yl)benzoic acid. A solution of tert-butyl 4-((6-(difluoromethyl)-3-(4-(methoxycarbonyl)phenyl)-hexahydro-1H-pyrrolo(1,2-a)pyrazin-2-yl)methyl)-5-methoxy-7-methylindole-1-carboxylate (10 mg, 0.02 mmol) and KOH (10 mg, 0.18 mmol) in EtOH (0.2 mL) and H₂O (0.1 mL) was stirred for 1 h at 80° C. The mixture was acidified to pH=6 with sat. citric acid. The residue was purified by reverse phase flash with the following conditions (acetonitrile, 5-34%/10 mM NH₄HCO₃ in water) to afford 4-(6-(difluoromethyl)-2-((5-methoxy-7-methyl-1H-indol-4-yl)methyl)-hexahydro-1H-pyrrolo(1,2-a)pyrazin-3-yl)benzoic acid (2.0 mg, 24.86% yield) as a white solid. LCMS (ESI): [M+H]⁺=470.2; ¹H NMR (300 MHz, Methanol-d₄) δ 8.12 (d, J=8.1 Hz, 2H), 7.66 (d, J=7.7 Hz, 2H), 7.27 (d, J=3.1 Hz, 1H), 6.74 (s, 1H), 6.37 (d, J=3.2 Hz, 1H), 5.74 (d, J=5.2 Hz, 1H), 4.11 (t, J=11.7 Hz, 2H), 3.81 (d, J=12.2 Hz, 1H), 3.76 (s, 3H), 3.30 (d, J=7.8 Hz, 2H), 2.90-2.77 (m, 1H), 2.76-2.56 (m, 3H), 2.50 (s, 3H), 2.03-1.72 (m, 3H), 1.44 (t, J=10.5 Hz, 1H).

### Example 53. 4-((3R,7R,8aS)-7-Fluoro-2-((5-methoxy-7-methyl-1H-indol-4-yl)methyl)octahydropyrrolo[1,2-a]pyrazin-3-yl)benzoic acid

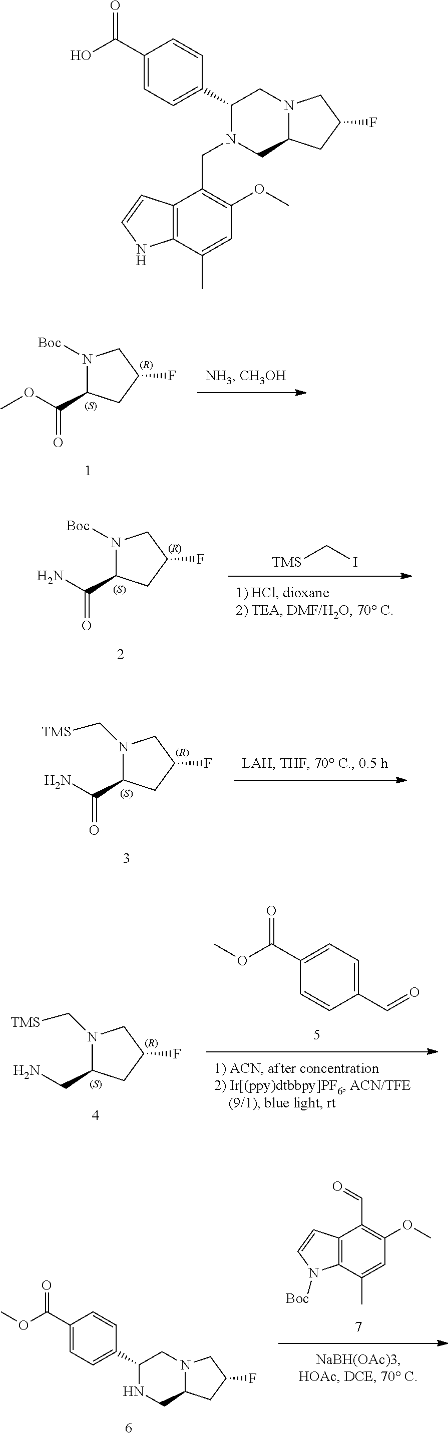

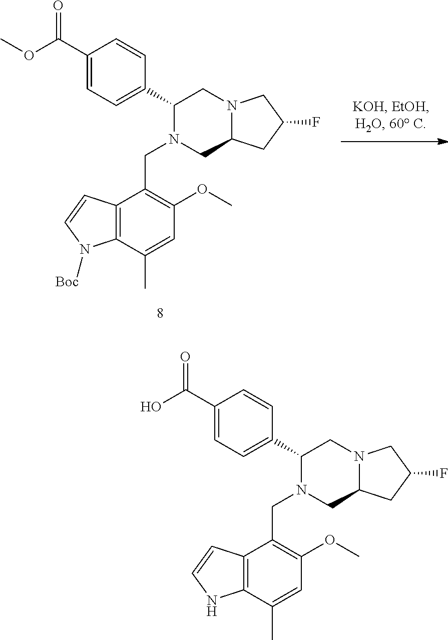 [SMILES: COC(=O)c1ccc([C@@H]2CN3C[C@H](F)C[C@H]3CN2Cc2c(OC)cc(C)c3c2ccn3C(=O)OC(C)(C)C)cc1 ; COc1cc(C)c2[nH]ccc2c1CN1C[C@@H]2C[C@@H](F)CN2C[C@H]1c1ccc(C(=O)O)cc1]

Step 1: tert-Butyl(2S,4R)-2-carbamoyl-4-fluoropyrrolidine-1-carboxylate tert-butyl (2S,4R)-2-carbamoyl-4-fluoropyrrolidine-1-carboxylate. A solution of 1-tert-butyl 2-methyl (2S,4R)-4-fluoropyrrolidine-1,2-dicarboxylate (15.0 g, 60.72 mmol) in 7 M NH₃ in MeOH (150 mL) was stirred for 12 h at room temperature. The resulting mixture was concentrated under reduced pressure to afford the title compound (13.5 g, 95.9% yield) as a yellow oil. LCMS (ESI): [M+H]⁺=233.1.

Step 2: (2S,4R)-4-Fluoro-1-((trimethylsilyl)methyl)pyrrolidine-2-carboxamide. A solution of tert-butyl(2S,4R)-2-carbamoyl-4-fluoropyrrolidine-1-carboxylate (8.0 g, 34.48 mmol) in DCM (80 mL) was treated with 4 M HCl in 1,4-dioxane (40 mL) for 1 h at room temperature. The resulting mixture was concentrated under reduced pressure. The crude product was directly used in the next step without further purification.

To the above mixture was added (iodomethyl)trimethylsilane (7.4 g, 34.57 mmol), DMF (80 mL), H₂O (3 mL) and TEA (10.4 g, 102.97 mmol) at room temperature. The resulting mixture was stirred for 12 h at 70° C. The reaction was quenched with water and extracted with ethyl acetate. The solvent was removed under vacuum. The resulting residue was purified by reverse phase chromatography (acetonitrile, 5-50%/10 mM NH₄HCO₃ in water) to afford the title compound (4.7 g, 62.7% yield) as a yellow oil. LCMS (ESI): [M+H]⁺=219.1.

Step 3: ((2S,4R)-4-Fluoro-1-((trimethylsilyl)methyl)pyrrolidin-2-yl)methanamine. A solution of (2S,4R)-4-fluoro-1-((trimethylsilyl)methyl)pyrrolidine-2-carboxamide (1.8 g, 8.25 mmol) in THF (500 mL) was added Lithium Aluminum Hydride (1.5 g, 39.5 mmol) at 0° C. and stirred for 30 min at 70° C. The reaction was quenched with water (1.5 mL), NaOH (15% wt in water, 1.5 mL) and water (4.5 mL) at 0° C. The resulting mixture was filtered, the filter cake was washed with THF. The filtrate was concentrated under reduced pressure to afford the title compound (1.4 g, 83.3% yield) as a light yellow oil. LCMS (ESI): [M+H]⁺=205.1.

Step 4: Methyl 4-((3R,7R,8aS)-7-fluoro-octahydropyrrolo(1,2-a)pyrazin-3-yl)benzoate. A solution of 1-((2S,4R)-4-fluoro-1-((trimethylsilyl)methyl)pyrrolidin-2-yl)methanamine (671 mg, 3.29 mmol) and methyl 4-formylbenzoate (539 mg, 3.28 mmol) in acetonitrile (4.5 mL) was stirred for 3 h at room temperature under nitrogen atmosphere. To the above mixture was added (Ir(dtbbpy)(ppy)₂)(PF₆) (300 mg, 0.33 mmol) and Difluoroethanol (1 mL) in portions at room temperature. The resulting mixture was stirred in blue light for additional 3 h at room temperature under nitrogen atmosphere. The solvent was removed under vacuum. The crude product was purified by reverse phase flash with the following conditions (acetonitrile, 5-24%/10 mM NH₄HCO₃ in water) to afford the title compound (57 mg, 6.2% yield) as a light yellow solid. LCMS (ESI): [M+H]⁺=279.1.

Step 5: tert-Butyl 4-(((3R,7R,8aS)-7-fluoro-3-(4-(methoxycarbonyl)phenyl)-hexahydro-1H-pyrrolo(1,2-a)pyrazin-2-yl)methyl)-5-methoxy-7-methylindole-1-carboxylate. A solution of methyl 4-((3R,7R,8aS)-7-fluoro-octahydropyrrolo(1,2-a)pyrazin-3-yl)benzoate (52 mg, 0.19 mmol), tert-butyl 4-formyl-5-methoxy-7-methylindole-1-carboxylate (65 mg, 0.22 mmol), HOAc (34 mg, 0.57 mmol) and Sodium triacetoxyborohydride (143 mg, 0.67 mmol) in 1,2-dichloroethane (1 mL) was stirred for 24 h at 70° C. The solvent was removed under reduced pressure. The residue was purified by reverse phase flash with the following conditions (acctonitrile, 5-85%/10 mM NH₄HCO₃ in water) to afford the title compound (60 mg, 58.2% yield) as a light yellow solid. LCMS (ESI): [M+H]⁺=552.3.

Step 6: 4-((3R,7R,8aS)-7-Fluoro-2-((5-methoxy-7-methyl-1H-indol-4-yl)methyl)octahydropyrrolo[1,2-a]pyrazin-3-yl)benzoic acid. A mixture of tert-butyl 4-(((3R,7R,8aS)-7-fluoro-3-(4-(methoxycarbonyl)phenyl)-hexahydro-1H-pyrrolo(1,2-a)pyrazin-2-yl)methyl)-5-methoxy-7-methylindole-1-carboxylate (55 mg, 0.10 mmol) and KOH (56 mg, 1.00 mmol) in EtOH (0.6 mL) and H₂O (0.3 mL) was stirred for 4 h at 60° C. The mixture was acidified to pH 6 with sat. citric acid. The solvent was removed. The residue was purified by reverse phase flash with the following conditions (acetonitrile, 5-20%/10 mM NH₄HCO₃ in water) to afford the title compound (19.6 mg, 44.4% yield) as a white solid. LCMS (ESI): [M+H]⁺=438.1; ¹H NMR (400 MHz, DMSO-d₆) δ 12.89 (s, 1H), 10.86 (t, J=2.4 Hz, 1H), 7.98 (d, J=8.0 Hz, 2H), 7.69 (d, J=7.8 Hz, 2H), 7.27 (t, J=2.8 Hz, 1H), 6.67 (s, 1H), 6.49-6.43 (m, 1H), 5.36-4.91 (m, 1H), 3.71 (s, 3H), 3.58 (d, J=11.9 Hz, 1H), 3.53-3.44 (m, 1H), 3.42-3.38 (m, 1H), 3.27 (d, J=11.8 Hz, 1H), 2.96-2.89 (m, 1H), 2.83-2.73 (m, 1H), 2.43 (s, 3H), 2.36-2.24 (m, 3H), 1.90-1.71 (m, 2H), 1.64-1.45 (m, 1H).

### Example 54. 4-((3R,7S,8aS)-7-Fluoro-2-((5-methoxy-7-methyl-1H-indol-4-yl)methyl)-hexahydro-1H-pyrrolo(1,2-a)pyrazin-3-yl)benzoic acid

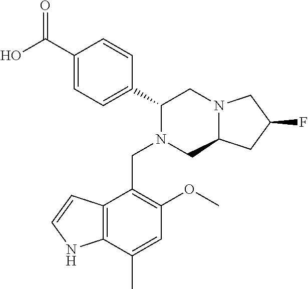 [SMILES: COc1cc(C)c2[nH]ccc2c1CN1C[C@@H]2C[C@H](F)CN2C[C@H]1c1ccc(C(=O)O)cc1]

The compound was prepared according to above.

4-((3R,7S,8aS)-7-Fluoro-2-((5-methoxy-7-methyl-1H-indol-4-yl)methyl)octahydropyrrolo[1,2-a]pyrazin-3-yl)benzoic acid (43 mg, 43.4% yield) as a light yellow solid. LCMS (ESI): [M+H]⁺=438.3, ¹H NMR (300 MHz, Methanol-d₄) δ 8.13-8.05 (m, 2H), 7.67 (d, J=7.8 Hz, 2H), 7.24 (d, J=3.1 Hz, 1H), 6.71 (s, 1H), 6.39 (d, J=3.1 Hz, 1H), 5.23-5.05 (m, 1H), 4.02 (d, J=12.1 Hz, 2H), 3.75 (s, 4H), 3.31-3.16 (m, 3H), 2.69-2.54 (m, 1H), 2.54-2.43 (m, 5H), 2.42-2.25 (m, 2H), 1.70-1.49 (m, 1H), 1.29 (s, 1H).

### Example 55. 4-((3R,8aS)-2-((5-Methoxy-7-methyl-1H-indol-4-yl)methyl)octahydropyrrolo[1,2-a]pyrazin-3-yl)-2-(methylamino)benzoic acid

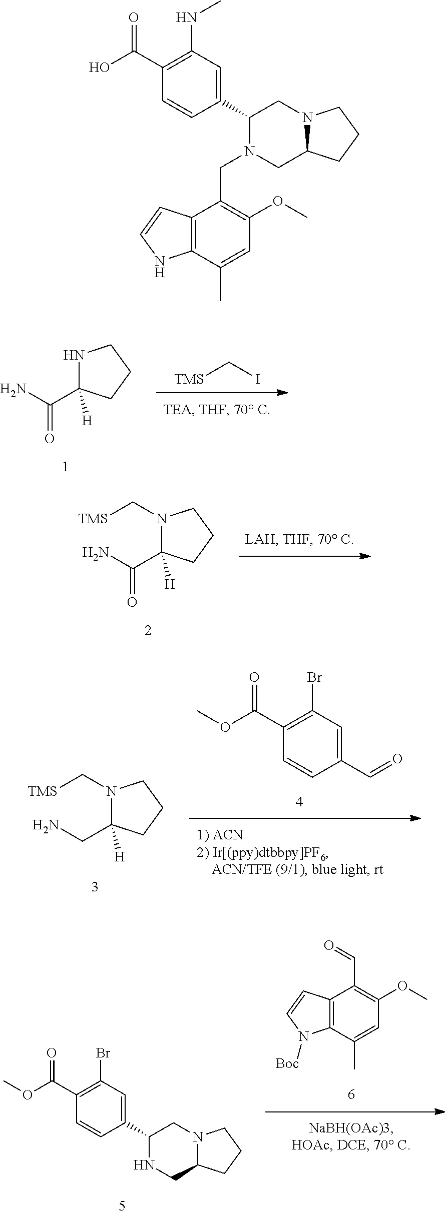

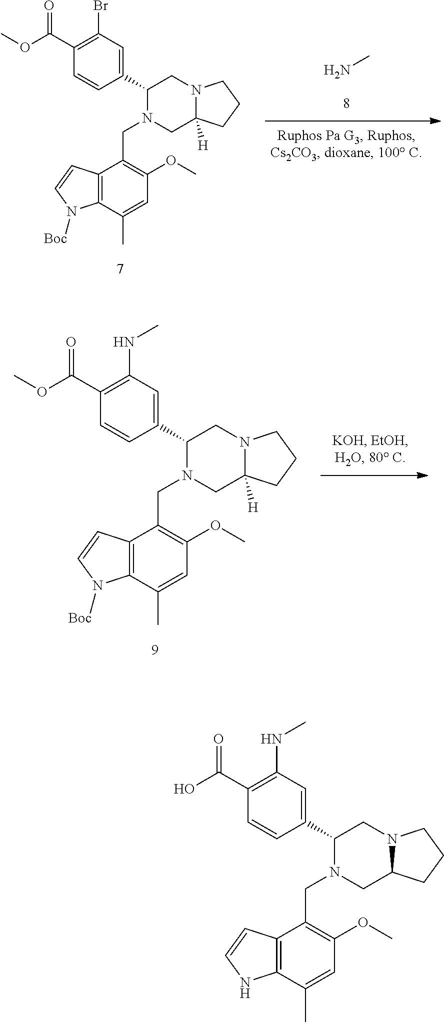

Step 1:(2S)-1-((Trimethylsilyl)methyl)pyrrolidine-2-carboxamide. A solution of prolinamide (10.00 g, 87.61 mmol) in THF (60 mL) was added (iodomethyl)trimethylsilane (37.52 g, 175.21 mmol) and Triethylamine (26.60 g, 262.82 mmol) and stirred at 70° C. under nitrogen atmosphere for overnight. The reaction was cooled to room temperature and condensed under vacuo. The residue was purified by reverse phase chromatography (Acetonitrile 0-40%/0.1% Ammonium bicarbonate in water) to afford the title compound (15.70 g, 89.4% yield) as a dark yellow oil. LCMS (ESI): [M+H]⁺=201.1.

Step 2: 1-((2S)-1-((Trimethylsilyl)methyl)pyrrolidin-2-yl)methanamine. To a solution of (2S)-1-((trimethylsilyl)methyl)pyrrolidine-2-carboxamide (2.00 g, 9.98 mmol) in THF (40 mL) was added Lithium Aluminum Hydride (1.14 g, 29.95 mmol) at 0° C. The mixture was stirred for 15 min and the mixture was allowed to warm to 70° C. stirred for 5 hours. The reaction mixture was quenched by water, 15% NaOH(aq) and water. The solids were removed. The filtrate was concentrated under vacuum to afford the title compound (1.56 g, 83.8% yield). LCMS (ESI): [M+H]⁺=187.1.

Step 3: Methyl 4-((3R,8aS)-octahydropyrrolo(1,2-a)pyrazin-3-yl)-2-bromobenzoate. A solution of 1-((2S)-1-((trimethylsilyl)methyl)pyrrolidin-2-yl)methanamine (1.00 g, 5.36 mmol) and methyl 2-bromo-4-formylbenzoate (1.30 g, 5.37 mmol) in ACN (9 mL) was stirred for 18 h at room temperature. Then Difluoroethanol (1 mL) and (Ir(dtbbpy)(ppy)₂)(PF₆) (490 mg, 0.54 mmol) was added and the reaction continued to stirred at room temparature for 3 hours under blue LED. The solvent was removed and the residue was purified by reverse phase chromatography (Acetonitrile 0-30%/0.1% Ammonium bicarbonate in water) to afford the title compound (200 mg, 10.9% yield) as a yellow oil. LCMS (ESI): [M+H]⁺=339.1.

Step 4: tert-Butyl 4-(((3R,8aS)-3-(3-bromo-4-(methoxycarbonyl)phenyl)-hexahydro-1H-pyrrolo(1,2-a)pyrazin-2-yl)methyl)-5-methoxy-7-methylindole-1-carboxylate. A solution of methyl 4-((3R,8aS)-octahydropyrrolo(1,2-a)pyrazin-3-yl)-2-bromobenzoate (180 mg, 0.53 mmol) in dichloromethane (2 mL) was added tert-butyl 4-formyl-5-methoxy-7-methylindole-1-carboxylate (230 mg, 0.79 mmol), HOAc (32 mg, 0.53 mmol) and Sodium triacetoxyborohydride (225 mg, 1.06 mmol) at room temperature. The resulting mixture was stirred for 3 hours at 70° C. The solvent was concentrated under vacuum and the residue was purified by flash with Sodium triacetoxyborohydride (7/3) to afford the title compound (170 mg, 52.3% yield) as a yellow oil. LCMS (ESI): [M+H]⁺=612.2.

Step 5: tert-Butyl 4-(((3R,8aS)-3-(4-(methoxycarbonyl)-3-(methylamino)phenyl)-hexahydro-1H-pyrrolo(1,2-a)pyrazin-2-yl)methyl)-5-methoxy-7-methylindole-1-carboxylate. A solution of tert-butyl 4-(((3R,8aS)-3-(3-bromo-4-(methoxycarbonyl)phenyl)-hexahydro-1H-pyrrolo(1,2-a)pyrazin-2-yl)methyl)-5-methoxy-7-methylindole-1-carboxylate (130 mg, 0.21 mmol) in 1,4-dioxane (2 mL) was added 1 M methylamine in THF (1.06 mL, 1.06 mmol), RuPhos Pd G3 (18 mg, 0.02 mmol), RuPhos (20 mg, 0.04 mmol) and Cs₂CO₃ (207 mg, 0.64 mmol) and stirred for 18 hours at 100° C. under nitrogen atmosphere. The solvent was concentrated under vacuum and the residue was purified by silica gel column eluting with petroleum ether/ethyl acetate (0-95%) to afford the title compound (30 mg, 25.1% yield) as a yellow solid. LCMS (ESI): [M+H]⁺=563.3.

Step 6: 4-((3R,8aS)-2-((5-Methoxy-7-methyl-1H-indol-4-yl)methyl)octahydropyrrolo[1,2-a]pyrazin-3-yl)-2-(methylamino)benzoic acid. A solution of tert-butyl 4-(((3R,8aS)-3-(4-(methoxycarbonyl)-3-(methylamino)phenyl)-hexahydro-1H-pyrrolo(1,2-a)pyrazin-2-yl)methyl)-5-methoxy-7-methylindole-1-carboxylate (30 mg, 0.05 mmol) in EtOH (1 mL) was added KOH (60 mg, 1.06 mmol) in H₂O (0.2 mL) and stirred for 1.5 hours at 80° C. The mixture was allowed to cool down to room temperature and was neutralized to pH=6 with sat. Citric acid. The resulting residue was purified by reverse phase chromatography (Acetonitrile 0-30%/0.1% Ammonium bicarbonate in water) to afford the title compound (13.2 mg, 53.9% yield) as a white solid. LCMS (ESI): [M+H]⁺=449.2. ¹H NMR (300 MHz, DMSO-d₆) δ 10.84 (s, 1H), 7.80 (d, J=8.1 Hz, 1H), 7.26 (t, J=2.8 Hz, 1H), 6.96-6.75 (m, 2H), 6.67 (s, 1H), 6.62-6.53 (m, 1H), 3.71 (s, 4H), 3.18 (s, 2H), 2.93 (d, J=14.5 Hz, 5H), 2.77 (d, J=9.9 Hz, 1H), 2.42 (s, 3H), 2.19 (t, J=10.3 Hz, 1H), 2.08-1.79 (m, 3H), 1.75-1.52 (m, 3H), 1.21 (d, J=14.0 Hz, 1H).

### Example 56. 4-((2S)-1-((5-Methoxy-7-methyl-1H-indol-4-yl)methyl)-4,5-dimethylpiperazin-2-yl)benzoic acid & 4-((2R)-1-((5-Methoxy-7-methyl-1H-indol-4-yl)methyl)-4,5-dimethylpiperazin-2-yl)benzoic acid

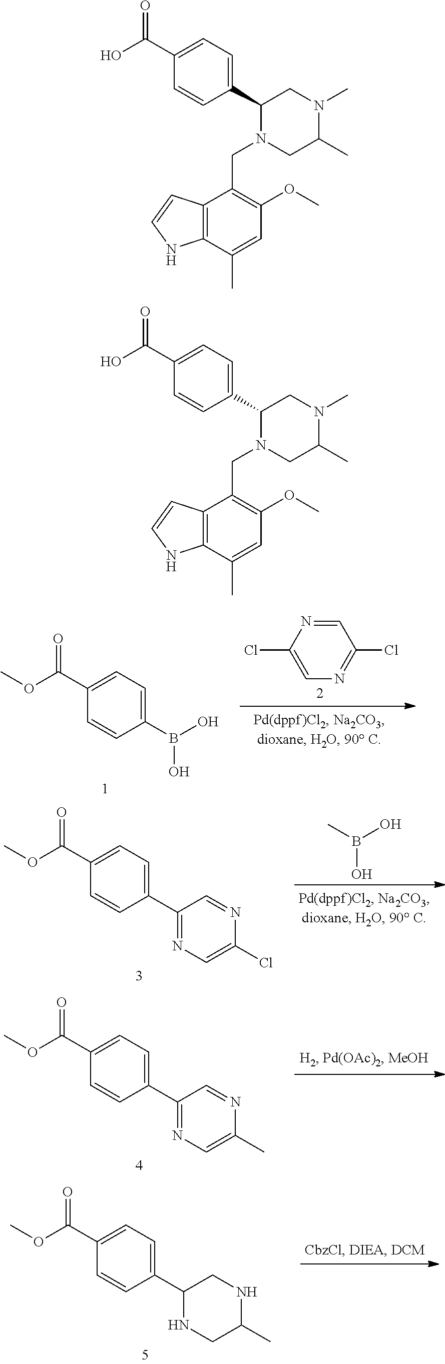

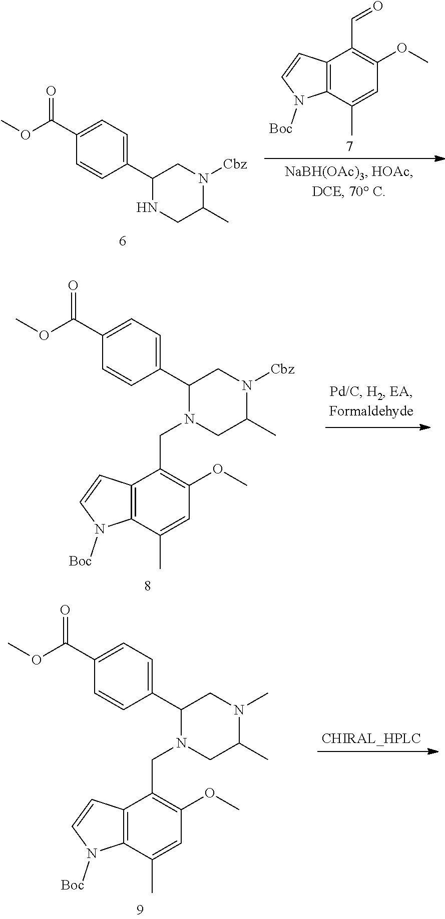

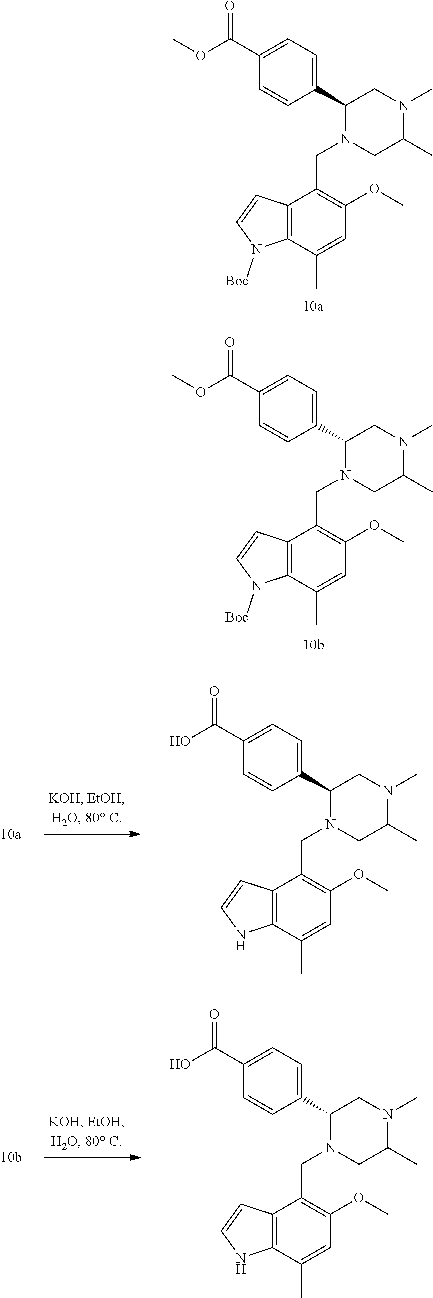

Step 1: methyl 4-(5-chloropyrazin-2-yl)benzoate. A mixture of 4-(methoxycarbonyl)phenylboronic acid (15 g, 83.34 mmol), 2,5-dichloropyrazine (18.62 g, 125.02 mmol), Pd(dppf)Cl₂ (6.10 g, 8.33 mmol) and Sodium carbonate (26.50 g, 250.04 mmol) in dioxane (150 mL) and water (30 mL) was stirred for 4 h at 90° C. under N₂. Then solids were filtered out. The filtrate was concentrated under reduced pressure. The residue was purified by silica gel column chromatography, eluted with petroleum ether/ethyl acetate (2:1) to afford the title compound (15 g, 72.3% yield) as a white solid. LCMS (ESI): [M+H]⁺=249.0.

Step 2: Methyl 4-(5-methylpyrazin-2-yl)benzoate. A solution of methyl 4-(5-chloropyrazin-2-yl)benzoate (7 g, 28.15 mmol) in 1,4-dioxane (70 mL) and water (14 mL) was added methylboronic acid (2.53 g, 42.22 mmol), Pd(dppf)Cl₂ (2.29 g, 2.81 mmol) and Sodium carbonate (8.95 g, 84.45 mmol) and stirred for 6 h at 90° C. under nitrogen atmosphere. Then solids were filtered out. The filtrate was concentrated under reduced pressure. The residue was purified by silica gel column chromatography, eluted with petroleum ether/ethyl acetate (74/26) to afford the title compound (2.7 g, 42.0% yield) as a red solid. LCMS (ESI): [M+H]⁺=229.2.

Step 3: Methyl 4-(5-methylpiperazin-2-yl)benzoate. A solution of methyl 4-(5-methylpyrazin-2-yl)benzoate (1.7 g, 7.44 mmol) in methanol (50 mL) was treated with Palladium (II) Acetate (836 mg, 3.72 mmol) for 18 hours at room temperature under Hydrogen. The solids were filtered out. The solvent was removed under vacuum to afford the title compound (1.6 g crude) as a yellow solid. The crude product was used in the next step directly without further purification. LCMS (ESI): [M+H]⁺=235.1.

Step 4: Benzyl 5-(4-(methoxycarbonyl)phenyl)-2-methylpiperazine-1-carboxylate. To a stirred mixture of methyl 4-(5-methylpiperazin-2-yl)benzoate (500 mg, 2.13 mmol) and triethylamine (647 mg, 6.40 mmol) in dichloromethane (20 mL) was added Benzyl chloroformate (327 mg, 1.92 mmol) in portions at 0° C. The resulting mixture was stirred for 2 h at room temperature. The resulting mixture was concentrated under reduced pressure. The resulting residue was purified by reverse phase chromatography (Acetonitrile 0-50%/0.1% Ammonium bicarbonate in water) to afford the title compound (430 mg, 27.4% yield) as a yellow oil. LCMS (ESI): [M+H]⁺=369.2.

Step 5: tert-Butyl 4-((4-((benzyloxy)carbonyl)-2-(4-(methoxycarbonyl)phenyl)-5-methylpiperazin-1-yl)methyl)-5-methoxy-7-methyl-1H-indole-1-carboxylate. To a stirred mixture of Benzyl 5-(4-(methoxycarbonyl)phenyl)-2-methylpiperazine-1-carboxylate (250 mg, 0.71 mmol) and tert-butyl 4-formyl-5-methoxy-7-methylindole-1-carboxylate (250 mg, 0.86 mmol) in dichloroethane (5 mL) were added Sodium triacetoxyborohydride (300 mg, 1.41 mmol) and HOAc (50 mg, 0.83 mmol). The resulting mixture was stirred for additional 3 h at 45° C. The resulting mixture was concentrated under reduced pressure. The residue was purified by reversed-phase flash chromatography with the following conditions: column, C18 silica gel; mobile phase, MeCN in Water (10 mmol/L NH4HCO₃), 70% to 85% gradient in 10 min; detector, UV 254 nm. to afford the title compound (230 mg, 51.7% yield) as a yellow solid. LCMS (ESI): [M+H]⁺=624.2.

Step 6: tert-Butyl 5-methoxy-4-((2-(4-(methoxycarbonyl)phenyl)-4,5-dimethylpiperazin-1-yl)methyl)-7-methyl-1H-indole-1-carboxylate. A mixture of tert-Butyl 4-((4-((benzyloxy)carbonyl)-2-(4-(methoxycarbonyl)phenyl)-5-methylpiperazin-1-yl)methyl)-5-methoxy-7-methyl-1H-indole-1-carboxylate (246 mg, 0.38 mmol) and 10% Pd/C (123 mg) in methanol (3 mL) and 37% formaldehyde in water (1 mL) was stirred for 1 h at room temperature under hydrogen atmosphere. The resulting mixture was concentrated under vacuum. The residue was purified by reversed-phase flash chromatography with the following conditions: column, C18 silica gel; mobile phase, MeCN in Water (10 mmol/L NH4HCO₃), 10% to 50% gradient in 10 min; detector, UV 254 nm. This resulted in the title compound (120 mg, 62% yield) as a yellow oil. LCMS (ESI): [M+H]⁺=522.3.

Step 7: tert-Butyl 5-methoxy-4-(((2S)-2-(4-(methoxycarbonyl)phenyl)-4,5-dimethylpiperazin-1-yl)methyl)-7-methyl-1H-indole-1-carboxylate (isomer 1, faster peak) & tert-Butyl 5-methoxy-4-(((2R)-2-(4-(methoxycarbonyl)phenyl)-4,5-dimethylpiperazin-1-yl)methyl)-7-methyl-1H-indole-1-carboxylate (isomer 2, slower peak).

tert-Butyl 5-methoxy-4-((2-(4-(methoxycarbonyl)phenyl)-4,5-dimethylpiperazin-1-yl)methyl)-7-methyl-1H-indole-1-carboxylate (120 mg, 0.23 mmol) was applied for further separation by Chiral HPLC.

tert-Butyl 5-methoxy-4-(((2S)-2-(4-(methoxycarbonyl)phenyl)-4,5-dimethylpiperazin-1-yl)methyl)-7-methyl-1H-indole-1-carboxylate (isomer 1, faster peak) (45 mg,37.5% yield) LCMS (ESI): [M+H]⁺=522.3. t_{R}=2.71 min (CHIRALPAK IG-3, 4.6*50 mm, 3 μm, Hex(0.1% DEA):IPA=95:5, 1.0 mL/min, 254 nm).

tert-Butyl 5-methoxy-4-(((2R)-2-(4-(methoxycarbonyl)phenyl)-4,5-dimethylpiperazin-1-yl)methyl)-7-methyl-1H-indole-1-carboxylate (isomer 2, slower peak) (35 mg, 29.1% yield) LCMS (ESI): [M+H]⁺=522.3. t_{R}=3.16 min (CHIRALPAK IG-3, 4.6*50 mm, 3 μm, Hex(0.1% DEA):IPA=95:5, 1.0 mL/min, 254 nm).

Step 7: 4-((2S)-1-((5-Methoxy-7-methyl-1H-indol-4-yl)methyl)-4,5-dimethylpiperazin-2-yl)benzoic acid & 4-((2R)-1-((5-Methoxy-7-methyl-1H-indol-4-yl)methyl)-4,5-dimethylpiperazin-2-yl)benzoic acid.

To a stirred mixture of tert-Butyl 5-methoxy-4-(((2S)-2-(4-(methoxycarbonyl)phenyl)-4,5-dimethylpiperazin-1-yl)methyl)-7-methyl-1H-indole-1-carboxylate (isomer 1, faster peak) (35 mg, 0.06 mmol) and KOH (100 mg, 1.78 mmol) in EtOH (3 mL) and H₂O (0.6 mL). The resulting mixture was stirred for 4 h at 60° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH4HCO₃), 10% to 50% gradient in 10 min; detector, UV 254 nm. to afford 4-((2S)-1-((5-Methoxy-7-methyl-1H-indol-4-yl)methyl)-4,5-dimethylpiperazin-2-yl)benzoic acid (20.9 mg, 76.4% yield) as a white solid. LCMS (ESI): [M+H]⁺=409.2. ¹H NMR (300 MHz, DMSO-d₆) δ 10.82 (s, 1H), 8.05-7.86 (m, 2H), 7.67 (d, J=7.9 Hz, 2H), 7.25 (t, J=2.8 Hz, 1H), 6.65 (s, 1H), 6.44 (dd, J=3.1, 1.9 Hz, 1H), 3.70 (s, 3H), 3.53 (d, J=11.9 Hz, 1H), 3.42-3.36 (m, 1H), 3.22-3.13 (m, 1H), 2.73-2.61 (m, 1H), 2.56 (d, J=8.8 Hz, 1H), 2.41 (s, 3H), 2.10 (s, 4H), 1.97-1.85 (m, 2H), 0.82 (d, J=5.1 Hz, 3H).

To a stirred mixture of tert-Butyl 5-methoxy-4-(((2R)-2-(4-(methoxycarbonyl)phenyl)-4,5-dimethylpiperazin-1-yl)methyl)-7-methyl-1H-indole-1-carboxylate (30 mg, 0.05 mmol) and KOH (100 mg, 1.78 mmol) in EtOH (3 mL) and water (0.6 mL). The resulting mixture was stirred for 4 h at 60° C. The reaction mixture was acidified to pH=6 with sat. citric acid solution. The solvent was removed under vacuum. The residue was purified by reversed-phase flash chromatography with the following conditions: column, C18 silica gel; mobile phase, MeCN in Water (10 mmol/L NH4HCO₃), 10% to 50% gradient in 10 min; detector, UV 254 nm. to afford 4-((2R)-1-((5-Methoxy-7-methyl-1H-indol-4-yl)methyl)-4,5-dimethylpiperazin-2-yl)benzoic acid (20.6 mg, 87.9% yield) as a white solid. LCMS (ESI): [M+H]⁺=409.2. ¹H NMR (300 MHz, DMSO-d₆) δ 10.82 (s, 1H), 8.05-7.86 (m, 2H), 7.67 (d, J=7.9 Hz, 2H), 7.25 (t, J=2.8 Hz, 1H), 6.65 (s, 1H), 6.44 (dd, J=3.1, 1.9 Hz, 1H), 3.70 (s, 3H), 3.53 (d, J=11.9 Hz, 1H), 3.42-3.36 (m, 1H), 3.22-3.13 (m, 1H), 2.73-2.61 (m, 1H), 2.56 (d, J=8.8 Hz, 1H), 2.41 (s, 3H), 2.10 (s, 4H), 1.97-1.85 (m, 2H), 0.82 (d, J=5.1 Hz, 3H).

### Example 57. 4-((3R,8aS)-7,7-Difluoro-2-((5-methoxy-7-methyl-1H-indol-4-yl)methyl)octahydropyrrolo[1,2-a]pyrazin-3-yl)-2-hydroxybenzoic acid

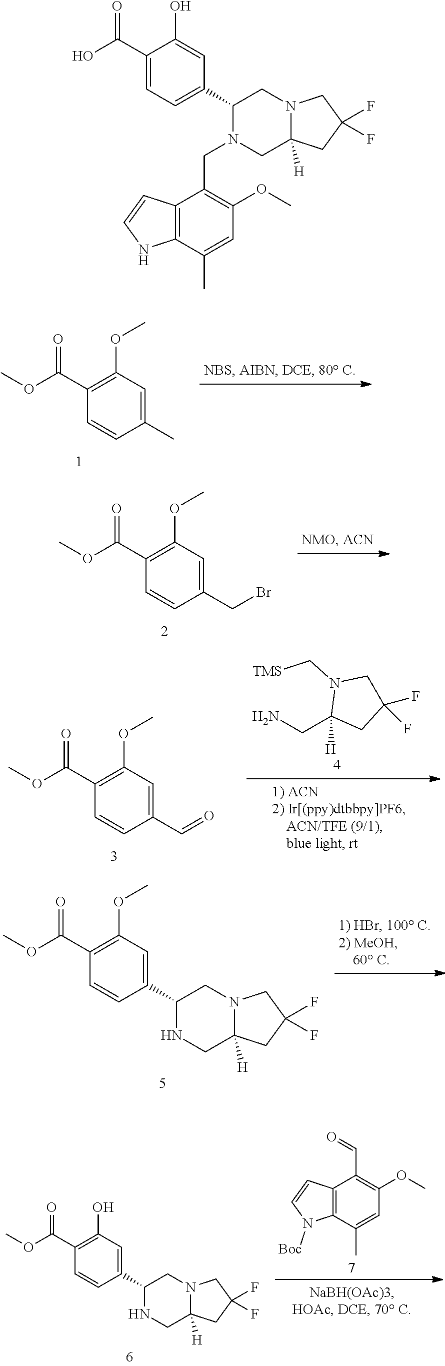

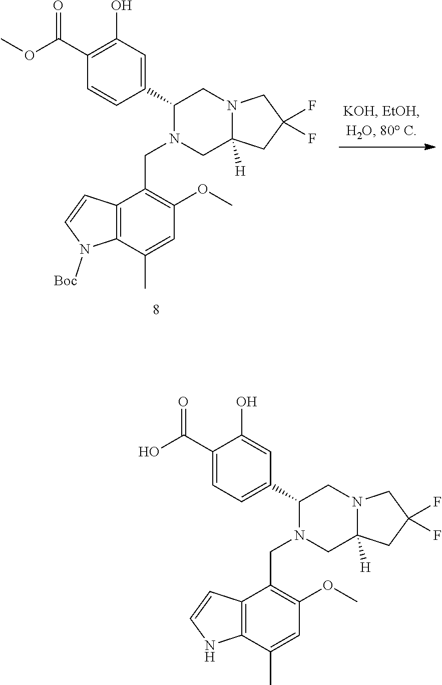 [SMILES: COC(=O)c1ccc([C@@H]2CN3CC(F)(F)C[C@H]3CN2Cc2c(OC)cc(C)c3c2ccn3C(=O)OC(C)(C)C)cc1O ; COc1cc(C)c2[nH]ccc2c1CN1C[C@@H]2CC(F)(F)CN2C[C@H]1c1ccc(C(=O)O)c(O)c1]

Step 1: Methyl 4-(bromomethyl)-2-methoxybenzoate. A mixture of methyl 2-methoxy-4-methylbenzoate (10 g, 55.49 mmol), NBS (9.88 g, 55.493 mmol) and AIBN (4.56 g, 27.747 mmol) in DCE (300 mL) was stirred for 4 h at 80° C. under nitrogen. The resulting mixture was quenched with water and extracted with ethyl acetate for 3 times. The solvent was concentrated under reduced pressure. The residue was purified by silica gel column chromatography, eluted with petroleum ether/ethyl acetate (5:1) to afford the title compound (8.4 g, 58.4% yield) as a colorless oil. LCMS (ESI): [M+H]⁺=259.0.

Step 2: Methyl 4-formyl-2-methoxybenzoate. A mixture of methyl 4-(bromomethyl)-2-methoxybenzoate (8.4 g, 32.42 mmol) and NMO (7.6 g, 64.84 mmol) in ACN (100 mL) was stirred for overnight at room temperature. The reaction was diluted with ethyl acatate, and washed with water for 3 times. The solvent was concentrated under reduced pressure. The residue was purified by silica gel column chromatography, eluted with dichloromethane/petroleum ether (1:1) to afford the title compound (1.6 g, 25.4% yield) as a colorless oil. LCMS (ESI): [M+H]⁺=195.1.

Step 3: Methyl 4-((3R,8aS)-7,7-difluorooctahydropyrrolo[1,2-a]pyrazin-3-yl)-2-methoxybenzoate. A mixture of 1-((2S)-4,4-difluoro-1-((trimethylsilyl)methyl)pyrrolidin-2-yl)methanamine (600 mg, 2.70 mmol) and methyl 4-formyl-2-methoxybenzoate (524 mg, 2.70 mmol) in ACN (12 mL) was stirred for overnight at rt. Then (Ir(dtbbpy)(ppy)₂)(PF₆) (247 mg, 0.27 mmol) and Difluoroethanol (1.3 mL) was added and stirred for 4 h at rt under nitrogen under blue LED. The solvent was removed under vacuum. The residue was purified by reverse flash chromatography with the following conditions: column, C18 silica gel; mobile phase, MeCN in Water (10 mmol/L NH4HCO₃), 10% to 40% gradient in 30 min; detector, UV 254 nm. The solvent was concentrated under reduced pressure to afford the title compound (210 mg, 23.9% yield) as a yellow oil. LCMS (ESI): [M+H]⁺=327.1.

Step 4: Methyl 4-((3R,8aS)-7,7-difluorooctahydropyrrolo[1,2-a]pyrazin-3-yl)-2-hydroxybenzoate. A mixture of methyl 4-((3R,8aS)-7,7-difluorooctahydropyrrolo[1,2-a]pyrazin-3-yl)-2-methoxybenzoate (190 mg, 0.58 mmol) in 40% HBr in water (10 mL) was stirred for 2 h at 100° C. The reaction was cooled to room temperature. Then MeOH (10 mL) was added and stirred for overnight at 60° C. The solvent was removed. The residue was purified by reverse flash chromatography with the following conditions: column, C18 silica gel; mobile phase, MeCN in Water (10 mmol/L NH4HCO₃), 20% to 50% gradient in 30 min; detector, UV 254 nm. The organic layer was concentrated under reduced pressure to afford the title compound (110 mg, 60.5% yield) as a yellow solid. LCMS (ESI): [M+H]⁺=313.1.

Step 5: tert-Butyl 4-(((3R,8aS)-7,7-difluoro-3-(3-hydroxy-4-(methoxycarbonyl)phenyl)hexahydropyrrolo[1,2-a]pyrazin-2 (1H)-yl)methyl)-5-methoxy-7-methyl-1H-indole-1-carboxylate. A mixture of methyl 4-((3R,8aS)-7,7-difluorooctahydropyrrolo[1,2-a]pyrazin-3-yl)-2-hydroxybenzoate (100 mg, 0.32 mmol), tert-butyl 4-formyl-5-methoxy-7-methylindole-1-carboxylate (140 mg, 0.48 mmol), AcOH (39 mg, 0.64 mmol) and NaBH(OAc)₃ (204 mg, 0.96 mmol) in dichloroethane (2 mL) was stirred for 4 h at 70° C. The resulting mixture was diluted with ethyl acatate. The residue was washed with water for 1 time. The organic layer was concentrated under reduced pressure. The residue was purified by silica gel column chromatography, eluted with petroleum ether/ethyl acetate (5:1) to afford the title compound (100 mg, 53.3% yield) as a light yellow solid. LCMS (ESI): [M+H]⁺=586.3.

Step 6: 4-((3R,8aS)-7,7-Difluoro-2-((5-methoxy-7-methyl-1H-indol-4-yl)methyl)octahydropyrrolo[1,2-a]pyrazin-3-yl)-2-hydroxybenzoic acid. A mixture of tert-butyl 4-(((3R,8aS)-7,7-difluoro-3-(3-hydroxy-4-(methoxycarbonyl)phenyl)hexahydropyrrolo[1,2-a]pyrazin-2 (1H)-yl)methyl)-5-methoxy-7-methyl-1H-indole-1-carboxylate (70 mg, 0.12 mmol) and KOH (67 mg, 1.2 mmol) in water (0.2 mL) and EtOH (2 mL) was stirred for 1 h at 70° C. The mixture was acidified to pH=6 with citric acid. The residue was purified by reversed-phase flash chromatography with the following conditions: column, C18 silica gel; mobile phase, MeCN in Water (10 mmol/L NH₄HCO₃), 10% to 35% gradient in 30 min; detector, UV 254 nm. The solvent was concentrated under reduced pressure to afford the compound (16.3 mg, 28.9% yield) as a white solid. LCMS (ESI): [M+H]⁺=472.2. ¹H NMR (300 MHz, Methanol-d₄) δ 8.02 (d, J=7.9 Hz, 1H), 7.33 (d, J=3.1 Hz, 1H), 7.15 (d, J=1.7 Hz, 1H), 7.04 (d, J=6.0 Hz, 1H), 6.78 (s, 1H), 6.39 (d, J=3.1 Hz, 1H), 4.45 (d, J=12.3 Hz, 2H), 4.14 (d, J=12.5 Hz, 1H), 3.82 (s, 3H), 3.60-3.42 (m, 2H), 3.29 (d, J=3.4 Hz, 1H), 3.15 (t, J=11.6 Hz, 1H), 2.97-2.60 (m, 3H), 2.52 (s, 3H), 2.42-2.22 (m, 1H), 2.17-1.90 (m, 1H).

### Example 58. 4-((3R,8aS)-2-((5-Methoxy-7-methyl-1H-indol-4-yl)methyl)octahydropyrrolo[1,2-a]pyrazin-3-yl)benzoic acid

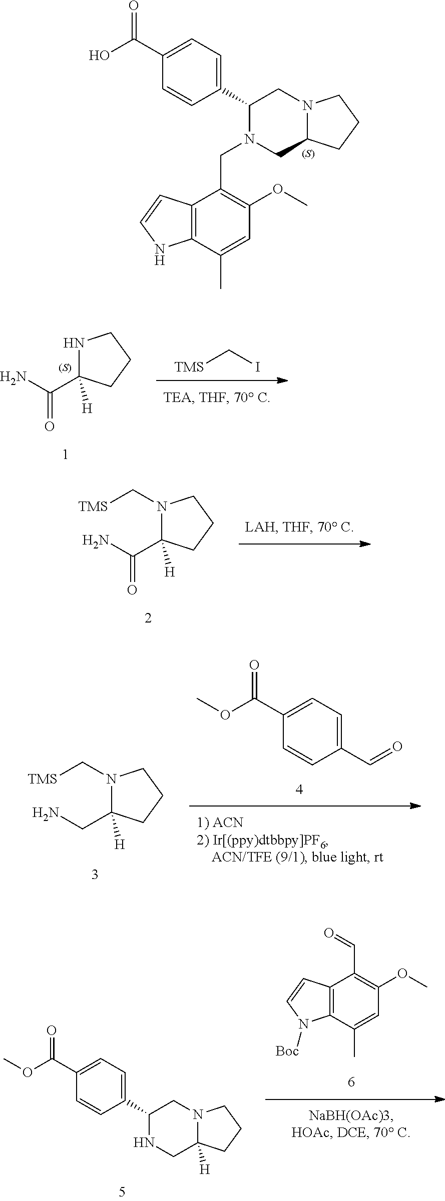

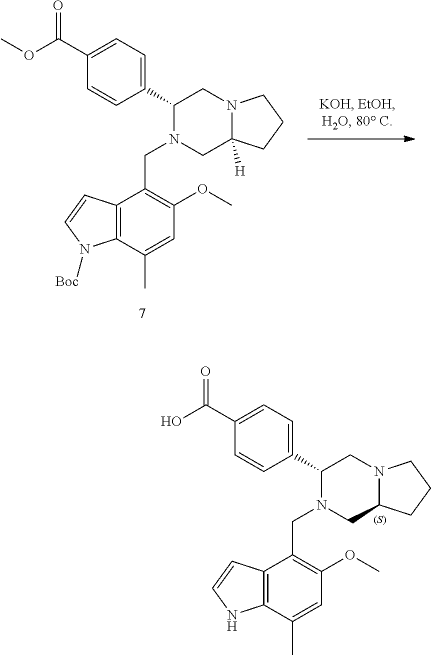

Step 1:(2S)-1-((Trimethylsilyl)methyl)pyrrolidine-2-carboxamide. A solution of prolinamide (5.00 g, 43.80 mmol) in THF (30 mL) was added (iodomethyl)trimethylsilane (18.76 g, 87.60 mmol) and TEA (13.30 g, 131.41 mmol) and stirred at 70° C. under nitrogen atmosphere for overnight. The reaction was cooled to room temperature and condensed under vacuo. The resulting residue was purified by reverse phase chromatography (Acetonitrile 0-40%/0.1% Ammonium bicarbonate in water) to afford the title compound (3.60 g, 41.0% yield) as a dark yellow oil. LCMS (ESI): [M+H]⁺=201.3.

Step 2: 1-((2S)-1-((Trimethylsilyl)methyl)pyrrolidin-2-yl)methanamine. To a solution of (2S)-1-((trimethylsilyl)methyl)pyrrolidine-2-carboxamide (2.00 g, 9.98 mmol) in THF (40 mL) was added Lithium Aluminum Hydride (1.14 g, 29.94 mmol) at 0° C. The mixture was stirred for 15 min and the mixture was allowed to warm to 70° C. and stirred for 5 hours. The reaction mixture was quenched by water, 15% NaOH(aq) and water. The solids were filtered out. The filtrate was concentrated to afford the title compound (1.56 g, 83.8% yield). LCMS (ESI): [M+H]⁺=187.0.

Step 3: Methyl 4-((3R,8aS)-octahydropyrrolo(1,2-a)pyrazin-3-yl)benzoate. A solution of 1-((2S)-1-((trimethylsilyl)methyl)pyrrolidin-2-yl)methanamine (700 mg, 3.76 mmol) and methyl 4-formylbenzoate (617 mg, 3.756 mmol) in ACN (3.6 mL) was stirred for 18 hours at room temperature. Then Difluoroethanol (0.4 mL) and (Ir(dtbbpy)(ppy)₂)(PF₆) (343 mg, 0.37 mmol) was added and the reaction was stirred for 4 hours under blue LED. The solvent was concentrated under vacuum and the residue was purified by reverse phase chromatography (Acetonitrile 0-48%/0.1% Ammonium bicarbonate in water) to afford the title compound (98 mg, 10.0% yield) as a yellow solid. LCMS (ESI): [M+H]⁺=261.1.

Step 4: tert-Butyl 4-(((3R,8aS)-3-(4-(methoxycarbonyl)phenyl)-hexahydro-1H-pyrrolo(1,2-a)pyrazin-2-yl)methyl)-5-methoxy-7-methylindole-1-carboxylate. A solution of methyl 4-((3R,8aS)-octahydropyrrolo(1,2-a)pyrazin-3-yl)benzoate (90 mg, 0.35 mmol) in DCE (2 mL) was added tert-butyl 4-formyl-5-methoxy-7-methylindole-1-carboxylate (120 mg, 0.41 mmol), HOAc (21 mg, 0.35 mmol) and NaBH(OAc)₃ (146 mg, 0.69 mmol) at room temperature. The resulting mixture was stirred for overnight at 70° C. The solvent was concentrated under vacuum. The residue was purified by silica gel column eluting with Dichloromethane/Methanol (0-9%) to afford the title compound (78 mg, 42.3% yield) as a yellow solid. LCMS (ESI): [M+H]⁺=534.3.

Step 5: 4-((3R,8aS)-2-((5-Methoxy-7-methyl-1H-indol-4-yl)methyl)octahydropyrrolo[1,2-a]pyrazin-3-yl)benzoic acid. A solution of tert-butyl 4-(((8aS)-3-(4-(methoxycarbonyl)phenyl)-hexahydro-1H-pyrrolo(1,2-a)pyrazin-2-yl)methyl)-5-methoxy-7-methylindole-1-carboxylate (78 mg, 0.15 mmol) in EtOH (3 mL) was added KOH (164 mg, 2.92 mmol) in H₂O (0.6 mL) and stirred for 1.5 hours at 80° C. The mixture was allowed to cool down to room temperature and was acided to pH=7 with sat. Citric acid. The resulting residue was purified by reverse phase chromatography (Acctonitrile 0-30%/0.1% Ammonium bicarbonate in water) to afford the title compound (41.1 mg, 63.7% yield) as a brown yellow solid. LCMS (ESI): [M+H]⁺=420.1. ¹H NMR (300 MHz, DMSO-d₆) δ 10.83 (s, 1H), 8.06-7.91 (m, 2H), 7.78-7.61 (d, J=7.9 Hz, 2H), 7.33-7.22 (t, J=2.8 Hz, 1H), 6.67 (s, 1H), 6.52-6.40 (m, 1H), 3.73 (s, 3H), 3.59 (d, J=11.8 Hz, 1H), 3.49-3.40 (m, 1H), 3.28 (d, J=11.8 Hz, 1H), 3.18 (s, 1H), 3.01-2.89 (m, 2H), 2.79 (d, J=8.0 Hz, 1H), 2.47-2.39 (m, 3H), 2.12 (t, J=10.5 Hz, 1H), 2.04-1.84 (m, 3H), 1.60 (d, J=8.4 Hz, 3H), 1.22 (d, J=11.4 Hz, 1H).

### Example 59. 5-Methoxy-7-methyl-4-((2-(4-(2H-1,2,3,4-tetrazol-5-yl)phenyl)-4-(2,2,2-trifluoroethyl)-1,4-diazepan-1-yl)methyl)-1H-indole

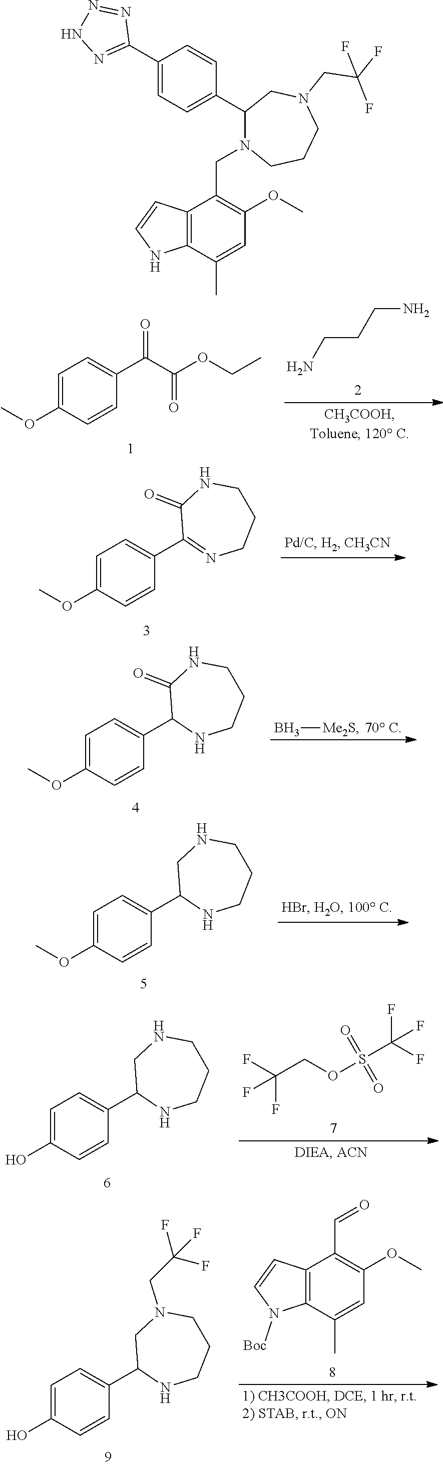

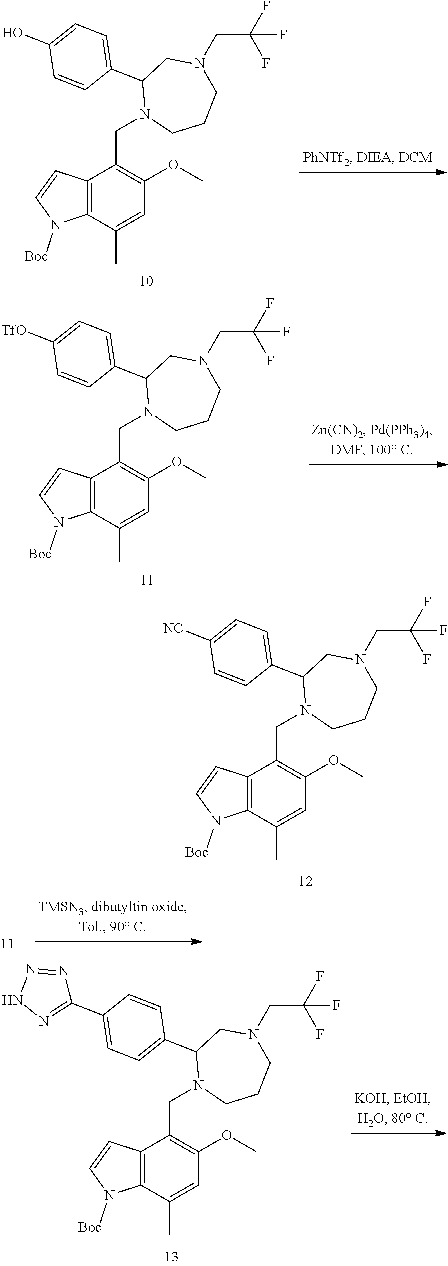 [SMILES: COc1cc(C)c2c(ccn2C(=O)OC(C)(C)C)c1CN1CCCN(CC(F)(F)F)CC1c1ccc(O)cc1 ; COc1cc(C)c2c(ccn2C(=O)OC(C)(C)C)c1CN1CCCN(CC(F)(F)F)CC1c1ccc(OS(=O)(=O)C(F)(F)F)cc1 ; COc1cc(C)c2c(ccn2C(=O)OC(C)(C)C)c1CN1CCCN(CC(F)(F)F)CC1c1ccc(C#N)cc1 ; COc1cc(C)c2c(ccn2C(=O)OC(C)(C)C)c1CN1CCCN(CC(F)(F)F)CC1c1ccc(-c2nn[nH]n2)cc1]

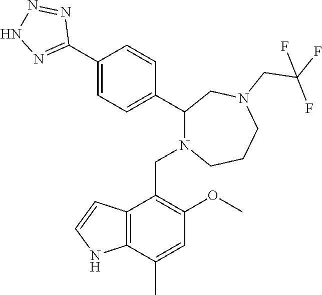 [SMILES: COc1cc(C)c2[nH]ccc2c1CN1CCCN(CC(F)(F)F)CC1c1ccc(-c2nn[nH]n2)cc1]

Step 1: 3-(4-Methoxyphenyl)-1,5,6,7-tetrahydro-1,4-diazepin-2-one. A mixture of ethyl 2-(4-methoxyphenyl)-2-oxoacetate (5.0 g, 24.02 mmol), α,ω-propanediamine (426.9 mg, 28.80 mmol) and HOAc (3.9 mL) in toluene (500 mL) was stirred for 24 h at 120° C. The resulting mixture was concentrated under reduced pressure to afford the title compound (5.2 g, 99.23% yield) as a black oil and as crude. LCMS (ESI): [M+H]⁺=219.1.

Step 2: 3-(4-Methoxyphenyl)-1,4-diazepan-2-one. A mixture of 3-(4-methoxyphenyl)-1,5,6,7-tetrahydro-1,4-diazepin-2-one (5.2 g, 23.83 mmol) and 10% Pd/C (10.1 g) in MeOH (250 mL) was stirred for 24 h at room temperature under hydrogen atmosphere. The crude product was purified by reverse phase flash with to afford the title compound (2.1 g, 40.0% yield) as a white solid. LCMS (ESI): [M+H]⁺=221.1.

Step 3: 4-(1,4-Diazepan-2-yl) phenol. A mixture of 3-(4-methoxyphenyl)-1,4-diazepan-2-one (2.1 g, 9.53 mmol) in 10 M BH₃-Me₂S (40 mL) was stirred for 4 h at 50° C. The reaction was quenched with MeOH at 0° C. The resulting mixture was concentrated under reduced pressure. To the above mixture was added 48% HBr in water (40 mL) and stirred for 24 h at 100° C. The resulting mixture was concentrated under reduced pressure to afford the title compound (1.5 g, 81.6% yield) as a black oil. LCMS (ESI): [M+H]⁺=193.2.

Step 4: 4-(4-(2,2,2-Trifluoroethyl)-1,4-diazepan-2-yl) phenol. A mixture of 4-(1,4-diazepan-2-yl) phenol (1.5 g, 7.80 mmol), 2,2,2-trifluoroethyl trifluoromethanesulfonate (1.8 g, 7.80 mmol) and N,N-diisopropylethylamine (5.0 g, 39.01 mmol) in DCM (20 mL) was stirred for 24 h at room temperature. The resulting mixture was concentrated under reduced pressure. The crude product was purified by reverse phase flash to afford the title compound (587 mg, 27.4% yield) as a white solid. LCMS (ESI): [M+H]⁺=274.2.

Step 5: tert-Butyl 4-((2-(4-hydroxyphenyl)-4-(2,2,2-trifluoroethyl)-1,4-diazepan-1-yl)methyl)-5-methoxy-7-methylindole-1-carboxylate. mixture of 4-(4-(2,2,2-trifluoroethyl)-1,4-diazepan-2-yl) phenol (587 mg, 2.14 mmol), tert-butyl 4-formyl-5-methoxy-7-methylindole-1-carboxylate (743 mg, 2.57 mmol) and HOAc (386 mg, 6.42 mmol) in 1,2-dichloroethane (6 mL) was stirred for overnight at 70° C. To the above mixture was added Sodium triacetoxyborohydride (908 mg, 4.28 mmol) at room temperature and stirred for 2 h at 70° C. The resulting mixture was extracted with ethyl acetate. The combined organic layers were washed with brine, dried over anhydrous sodium sulfate. After filtration, the filtrate was concentrated under reduced pressure. The residue was purified by flash chromatography on silica gel eluting with petroleum ether/ethyl acetate (3:2) to afford the title compound (500 mg, 42.6% yield) as a light yellow solid. LCMS (ESI): [M+H]⁺=548.6.

Step 6: tert-Butyl 5-methoxy-7-methyl-4-((4-(2,2,2-trifluoroethyl)-2-(4-(trifluoromethanesulfonyloxy)phenyl)-1,4-diazepan-1-yl)methyl) indole-1-carboxylate. A mixture of tert-butyl 4-((7-(4-hydroxyphenyl)-4-(2,2,2-trifluoroethyl)-1,4-diazepan-1-yl)methyl)-5-methoxy-7-methylindole-1-carboxylate (500 mg, 0.91 mmol), 1,1,1-trifluoro-N-phenyl-N-trifluoromethanesulfonylmethanesulfonamide (391 mg, 1.10 mmol) and N,N-diisopropylethylamine (354 mg, 2.74 mmol) in DCM (6 mL) was stirred for 4 h at 30° C. The solvent was removed under vacuum. The residue was purified by silica gel column chromatography, eluted with petroleum ether/ethyl acetate (9:1) to afford the title compound (416 mg, 67.03% yield) as a light yellow solid. LCMS (ESI): [M+H]⁺=680.6.

Step 7: tert-Butyl 4-((2-(4-cyanophenyl)-4-(2,2,2-trifluoroethyl)-1,4-diazepan-1-yl)methyl)-5-methoxy-7-methylindole-1-carboxylate. A mixture of tert-butyl 5-methoxy-7-methyl-4-((4-(2,2,2-trifluoroethyl)-2-(4-(trifluoromethanesulfonyloxy)phenyl)-1,4-diazepan-1-yl)methyl) indole-1-carboxylate (416 mg, 0.61 mmol), Zn(CN)₂ (144 mg, 1.22 mmol) and Pd(PPh₃)₄ (71 mg, 0.06 mmol) in DMF (5 mL) was stirred for 2 h at 100° C. under nitrogen atmosphere. The reaction was quenched with water and extracted with ethyl acetate. The combined organic layers were washed with brine, dried over anhydrous Na₂SO₄. After filtration, the filtrate was concentrated under reduced pressure. The residue was purified by silica gel column chromatography, eluted with petroleum ether/ethyl acetate (9:1) to afford the title compound (240 mg, 70.4% yield) as a light yellow solid. LCMS (ESI): [M+H]⁺=557.6.

Step 8: tert-Butyl 5-methoxy-7-methyl-4-((2-(4-(2H-1,2,3,4-tetrazol-5-yl)phenyl)-4-(2,2,2-trifluoroethyl)-1,4-diazepan-1-yl)methyl) indole-1-carboxylate. A mixture of tert-butyl 4-((2-(4-cyanophenyl)-4-(2,2,2-trifluoroethyl)-1,4-diazepan-1-yl)methyl)-5-methoxy-7-methylindole-1-carboxylate (240 mg, 0.43 mmol), azidotrimethylsilane (745 mg, 6.46 mmol) and dibutyltin oxide (107 mg, 0.43 mmol) in toluene (3 mL) was stirred for 3.5 h at 90° C. under nitrogen atmosphere. The resulting mixture was concentrated under reduced pressure. The residue was purified by flash chromatography on silica gel eluting with dichloromethane/methanol (7:1) to afford the title compound (178 mg, 68.8% yield) as a reddish brown solid. LCMS (ESI): [M+H]⁺=600.6.

Step 9: 5-Methoxy-7-methyl-4-((2-(4-(2H-1,2,3,4-tetrazol-5-yl)phenyl)-4-(2,2,2-trifluoroethyl)-1,4-diazepan-1-yl)methyl)-1H-indole. A mixture of tert-butyl 5-methoxy-7-methyl-4-((2-(4-(2H-1,2,3,4-tetrazol-5-yl)phenyl)-4-(2,2,2-trifluoroethyl)-1,4-diazepan-1-yl)methyl) indole-1-carboxylate (178 mg, 0.30 mmol) and KOH (167 mg, 2.97 mmol) in EtOH (1.4 mL) and water (0.7 mL) was stirred for 2 h at 70° C. The mixture was acidified to pH=6 with sat. citric acid. The solvent was removed. The residue was purified by reverse phase flash with the following conditions (acetonitrile, 5-37%/10 mM NH₄HCO₃ in water) to afford the compound (65 mg, 43.8% yield) as a white solid. LCMS (ESI): [M+H]⁺=500.2; ¹H NMR (300 MHz, DMSO-d₆) δ 10.90 (s, 1H), 8.10-8.02 (m, 2H), 7.69 (d, J=8.0 Hz, 2H), 7.25 (t, J=2.8 Hz, 1H), 6.68 (s, 1H), 6.25-6.22 (m, 1H), 4.04-3.73 (m, 3H), 3.68 (s, 3H), 3.45 (m, 3H), 3.07-2.75 (m, 5H), 2.42 (s, 3H), 2.95-2.55 (m, 2H).

### Example 60. 2-Fluoro-4-(1-((5-methoxy-7-methyl-1H-indol-4-yl)methyl)-4-methylpiperazin-2-yl)benzoic acid

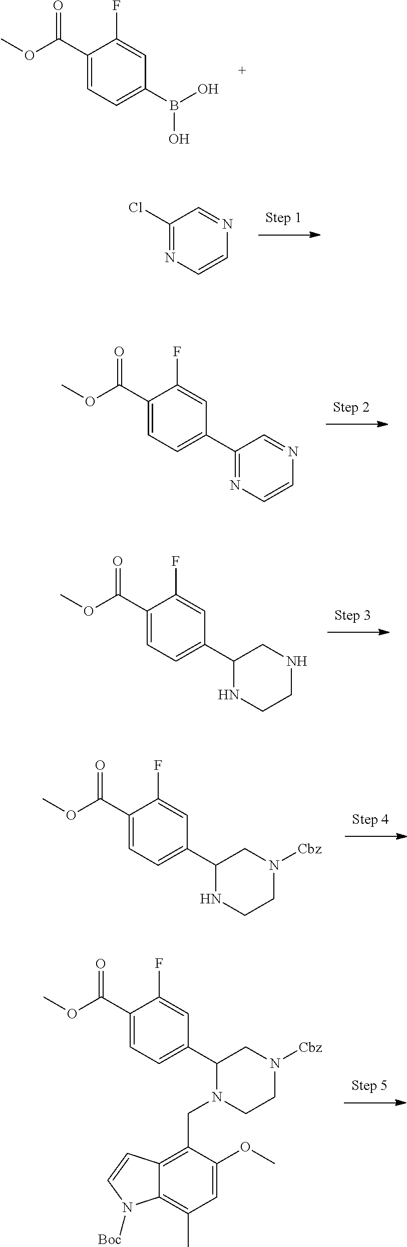 [SMILES: COC(=O)c1ccc(B(O)O)cc1F ; Clc1cnccn1 ; COC(=O)c1ccc(-c2cnccn2)cc1F ; COC(=O)c1ccc(C2CNCCN2)cc1F ; COC(=O)c1ccc(C2CN(C(=O)OCc3ccccc3)CCN2)cc1F ; COC(=O)c1ccc(C2CN(C(=O)OCc3ccccc3)CCN2Cc2c(OC)cc(C)c3c2ccn3C(=O)OC(C)(C)C)cc1F]

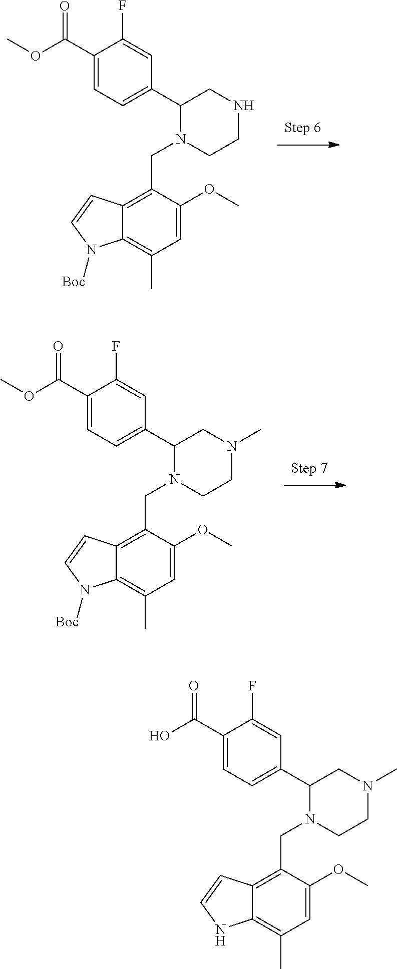

Step 1. Methyl 2-fluoro-4-(pyrazin-2-yl)benzoate was prepared following General Experimental Procedure 1. (3-Fluoro-4-(methoxycarbonyl)phenyl) boronic acid (25.1 g, 127 mmol) and 2-chloropyrazine (15.5 g, 135 mmol) gave methyl 2-fluoro-4-(pyrazin-2-yl)benzoate (23.0 g, 78%) as an off-white solid. 1H-NMR (299 MHz, CDCl₃) δ 9.07 (d, J=1.5 Hz, 1H), 8.68 (dd, J=2.5, 1.5 Hz, 1H), 8.60 (d, J=2.3 Hz, 1H), 8.08 (t, J=7.6 Hz, 1H), 7.97-7.76 (m, 2H), 3.97 (s, 3H).

Step 2. Methyl 2-fluoro-4-(piperazin-2-yl)benzoate was prepared following General Experimental Procedure 2. Methyl 2-fluoro-4-(pyrazin-2-yl)benzoate (23.0 g, 99 mmol) gave methyl 2-fluoro-4-(piperazin-2-yl)benzoate (15.3 g, 65%) as a yellow oil. LCMS (General 3 Basic) RT: 0.57 min; area % (254 nm): 100%; m/z=239.1 [M+H]⁺.

Step 3. Benzyl 3-(3-fluoro-4-(methoxycarbonyl)phenyl)piperazine-1-carboxylate was prepared following General Experimental Procedure 3. Methyl 2-fluoro-4-(piperazin-2-yl)benzoate (5.32 g, 22.3 mmol) gave benzyl 3-(3-fluoro-4-(methoxycarbonyl)phenyl)piperazine-1-carboxylate (4.37 g, 53%). LCMS (General 3 Basic) RT: 1.55 min; area % (254 nm): 100%; m/z=373.2 [M+H]⁺.

Step 4. tert-Butyl 4-((4-((benzyloxy)carbonyl)-2-(3-fluoro-4-(methoxycarbonyl)phenyl)piperazin-1-yl)methyl)-5-methoxy-7-methyl-1H-indole-1-carboxylate was prepared following General Experimental Procedure 4. Benzyl 3-(3-fluoro-4-(methoxycarbonyl)phenyl)piperazine-1-carboxylate (1.01 g, 2.71 mmol) and tert-butyl 4-formyl-5-methoxy-7-methyl-1H-indole-1-carboxylate (1.18 g, 4.08 mmol) gave tert-butyl 4-((4-((benzyloxy)carbonyl)-2-(3-fluoro-4-(methoxycarbonyl)phenyl)piperazin-1-yl)methyl)-5-methoxy-7-methyl-1H-indole-1-carboxylate (1.87 g, 57% purity, 61%). LCMS (General 3 Basic) RT: 2.42 min; area % (254 nm): 57%; m/z=646.3 [M+H]⁺.

Step 5. tert-Butyl 4-((2-(3-fluoro-4-(methoxycarbonyl)phenyl)piperazin-1-yl)methyl)-5-methoxy-7-methyl-1H-indole-1-carboxylate was prepared following General Experimental Procedure 5. tert-Butyl 4-((4-((benzyloxy)carbonyl)-2-(3-fluoro-4-(methoxycarbonyl)phenyl)piperazin-1-yl)methyl)-5-methoxy-7-methyl-1H-indole-1-carboxylate (1.64 g, 57 wt. %, 1.45 mmol) gave tert-butyl 4-((2-(3-fluoro-4-(methoxycarbonyl)phenyl)piperazin-1-yl)methyl)-5-methoxy-7-methyl-1H-indole-1-carboxylate (868 mg, >100%) as a white foam. LCMS (General 3 Basic) RT: 1.99 min; area % (254 nm): 100%; m/z=512.2 [M+H]⁺.

Step 6. tert-Butyl 4-((2-(3-fluoro-4-(methoxycarbonyl)phenyl)-4-methylpiperazin-1-yl)methyl)-5-methoxy-7-methyl-1H-indole-1-carboxylate was prepared following General Experimental Procedure 6. tert-Butyl 4-((2-(3-fluoro-4-(methoxycarbonyl)phenyl)piperazin-1-yl)methyl)-5-methoxy-7-methyl-1H-indole-1-carboxylate (95.3 mg, 186 μmol) and 37 wt. % aq. formaldehyde (83.2 μL, 930 μmol) gave crude tert-butyl 4-((2-(3-fluoro-4-(methoxycarbonyl)phenyl)-4-methylpiperazin-1-yl)methyl)-5-methoxy-7-methyl-1H-indole-1-carboxylate (105 mg, >100%)). LCMS (General 3 Basic) RT: 2.20 min; area % (254 nm): 97%; m/z=526.2 [M+H]⁺.

Step 7. 2-Fluoro-4-(1-((5-methoxy-7-methyl-1H-indol-4-yl)methyl)-4-methylpiperazin-2-yl)benzoic acid was prepared following General Experimental Procedure 8. Crude tert-butyl 4-((2-(3-fluoro-4-(methoxycarbonyl)phenyl)-4-methylpiperazin-1-yl)methyl)-5-methoxy-7-methyl-1H-indole-1-carboxylate (105 mg) gave 2-fluoro-4-(1-((5-methoxy-7-methyl-1H-indol-4-yl)methyl)-4-methylpiperazin-2-yl)benzoic acid (44.4 mg, 54% over two steps) as a white solid. LCMS (31697 LCMS-5 C3) RT: 1.561 min; area % (215 nm): 99.4%; m/z=412.2 [M+H]⁺. ¹H-NMR (400 MHz, DMSO) δ 10.85 (s, 1H), 7.87 (t, J=7.8 Hz, 1H), 7.51 (d, J=12.1 Hz, 1H), 7.46 (d, J=7.5 Hz, 1H), 7.27 (t, J=2.8 Hz, 1H), 6.67 (s, 1H), 6.43 (dd, J=2.9, 1.9 Hz, 1H), 3.72 (s, 3H), 3.62 (d, J=11.8 Hz, 1H), 3.44 (dd, J=10.3, 3.0 Hz, 1H), 3.21 (d, J=11.8 Hz, 1H), 3.17 (s, 1H), 2.72 (d, J=10.4 Hz, 1H), 2.62 (d, J=9.4 Hz, 2H), 2.42 (s, 3H), 2.24 (t, J=10.4 Hz, 1H), 2.14 (s, 3H), 1.99-1.87 (m, 2H).

### Example 61. 4-(1-((5-Methoxy-7-methyl-1H-indol-4-yl)methyl)-4-methylpiperazin-2-yl)-2-methylbenzoic acid

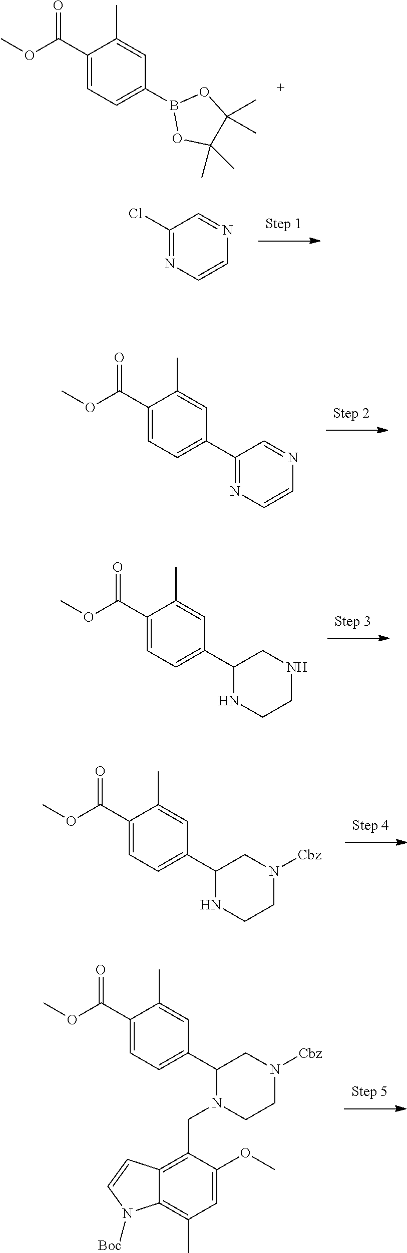

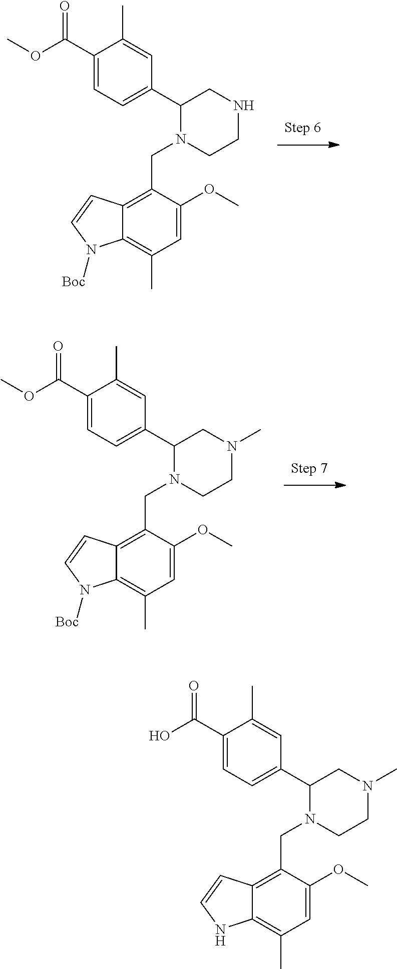

Step 1. Methyl 2-methyl-4-(pyrazin-2-yl)benzoate was prepared following General Experimental Procedure 1. Methyl 2-methyl-4-(4,4,5,5-tetramethyl-1,3,2-dioxaborolan-2-yl)benzoate (30.0 g, 109 mmol) and 2-chloropyrazine (13.0 g, 109 mmol) gave methyl 2-methyl-4-(pyrazin-2-yl)benzoate (6.50 g, 26%) as an off-white solid. ¹H-NMR (400 MHz, CDCl₃) δ 9.07 (d, J=1.6 Hz, 1H), 8.67 (dd, J=2.5, 1.6 Hz, 1H), 8.56 (d, J=2.5 Hz, 1H), 8.06 (d, J=8.1 Hz, 1H), 7.98-7.74 (m, 2H), 3.93 (s, 3H), 2.71 (s, 3H).

Step 2. Methyl 2-methyl-4-(piperazin-2-yl)benzoate was prepared following General Experimental Procedure 2. Methyl 2-methyl-4-(pyrazin-2-yl)benzoate (6.50 g, 28.4 mmol) gave methyl 2-methyl-4-(piperazin-2-yl)benzoate (5.91 g, 89%) as a yellow oil. LCMS (General 3 Basic) RT: 0.78 min; area % (254 nm): 98%; m/z=235.1 [M+H]⁺.

Step 3. Benzyl 3-(4-(methoxycarbonyl)-3-methylphenyl)piperazine-1-carboxylate was prepared following General Experimental Procedure 3. Methyl 2-methyl-4-(piperazin-2-yl)benzoate (5.91 g, 25.2 mmol) gave benzyl 3-(4-(methoxycarbonyl)-3-methylphenyl)piperazine-1-carboxylate (4.19 g, 45%). LCMS (General 3 Basic) RT: 1.63 min; area % (254 nm): 96%; m/z=369.3 [M+H]⁺.

Step 4. tert-Butyl 4-((4-((benzyloxy)carbonyl)-2-(4-(methoxycarbonyl)-3-methylphenyl)piperazin-1-yl)methyl)-5-methoxy-7-methyl-1H-indole-1-carboxylate was prepared following General Experimental Procedure 4. Benzyl 3-(4-(methoxycarbonyl)-3-methylphenyl)piperazine-1-carboxylate (1.04 g, 2.82 mmol) and tert-butyl 4-formyl-5-methoxy-7-methyl-1H-indole-1-carboxylate (1.23 g, 4.27 mmol) gave tert-butyl 4-((4-((benzyloxy)carbonyl)-2-(4-(methoxycarbonyl)-3-methylphenyl)piperazin-1-yl)methyl)-5-methoxy-7-methyl-1H-indole-1-carboxylate (1.46 g, 81%). LCMS (General 2 Basic) RT: 2.28 min; area % (254 nm): 57%; m/z=642.3 [M+H]⁺.

Step 5. tert-Butyl 5-methoxy-4-((2-(4-(methoxycarbonyl)-3-methylphenyl)piperazin-1-yl)methyl)-7-methyl-1H-indole-1-carboxylate was prepared following General Experimental Procedure 5. tert-Butyl 4-((4-((benzyloxy)carbonyl)-2-(4-(methoxycarbonyl)-3-methylphenyl)piperazin-1-yl)methyl)-5-methoxy-7-methyl-1H-indole-1-carboxylate (1.46 g, 2.27 mmol) gave tert-butyl 5-methoxy-4-((2-(4-(methoxycarbonyl)-3-methylphenyl)piperazin-1-yl)methyl)-7-methyl-1H-indole-1-carboxylate (1.12 g, 97%) as a white foam. LCMS (General 3 Basic) RT: 2.08 min; area % (254 nm): 97%; m/z=508.2 [M+H]⁺.

Step 6. tert-Butyl 5-methoxy-4-((2-(4-(methoxycarbonyl)-3-methylphenyl)-4-methylpiperazin-1-yl)methyl)-7-methyl-1H-indole-1-carboxylate was prepared following General Experimental Procedure 6. tert-Butyl 5-methoxy-4-((2-(4-(methoxycarbonyl)-3-methylphenyl)piperazin-1-yl)methyl)-7-methyl-1H-indole-1-carboxylate (95.4 mg, 188 μmol) and 37 wt. % aq. formaldehyde (84.0 μL, 1.13 mmol) gave crude tert-butyl 5-methoxy-4-((2-(4-(methoxycarbonyl)-3-methylphenyl)-4-methylpiperazin-1-yl)methyl)-7-methyl-1H-indole-1-carboxylate (98.9 mg, >100%). LCMS (General 3 Basic) RT: 2.30 min; area % (254 nm): 97%; m/z=522.3 [M+H]⁺.

Step 7. 4-(1-((5-Methoxy-7-methyl-1H-indol-4-yl)methyl)-4-methylpiperazin-2-yl)-2-methylbenzoic acid was prepared following General Experimental Procedure 8. Crude tert-butyl 5-methoxy-4-((2-(4-(methoxycarbonyl)-3-methylphenyl)-4-methylpiperazin-1-yl)methyl)-7-methyl-1H-indole-1-carboxylate (98.9 mg) gave 4-(1-((5-methoxy-7-methyl-1H-indol-4-yl)methyl)-4-methylpiperazin-2-yl)-2-methylbenzoic acid (44.2 mg, 57% over two steps) as a white solid. LCMS (31697 LCMS-5 C3) RT: 1.562 min; area % (215 nm): 99.8%; m/z=408.2 [M+H]⁺. ¹H-NMR (400 MHz, DMSO) δ 10.82 (t, J=2.3 Hz, 1H), 7.84 (d, J=8.0 Hz, 1H), 7.47 (s, 2H), 7.26 (t, J=2.8 Hz, 1H), 6.65 (s, 1H), 6.43 (dd, J=3.1, 1.9 Hz, 1H), 3.71 (s, 3H), 3.59 (d, J=11.8 Hz, 1H), 3.35-3.29 (m, 4H), 3.21 (d, J=11.8 Hz, 2H), 2.67 (p, J=1.8 Hz, 1H), 2.65-2.55 (m, 5H), 2.42 (s, 3H), 2.24-2.15 (m, 1H), 2.11 (s, 3H), 1.99-1.85 (m, 2H).

### Example 62. 2-Hydroxy-4-(1-((5-methoxy-7-methyl-1H-indol-4-yl)methyl)-4-methylpiperazin-2-yl)benzoic

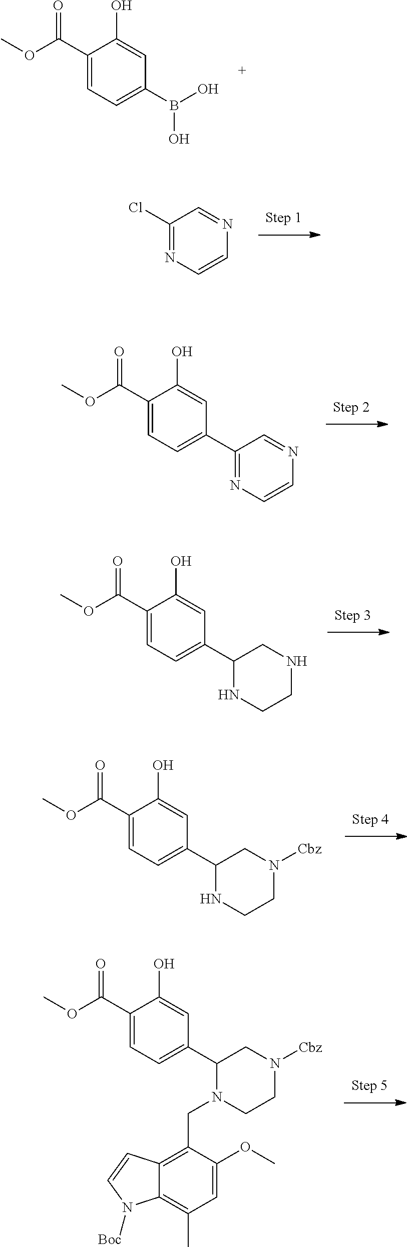 [SMILES: COC(=O)c1ccc(B(O)O)cc1O ; Clc1cnccn1 ; COC(=O)c1ccc(-c2cnccn2)cc1O ; COC(=O)c1ccc(C2CNCCN2)cc1O ; COC(=O)c1ccc(C2CN(C(=O)OCc3ccccc3)CCN2)cc1O ; COC(=O)c1ccc(C2CN(C(=O)OCc3ccccc3)CCN2Cc2c(OC)cc(C)c3c2ccn3C(=O)OC(C)(C)C)cc1O]

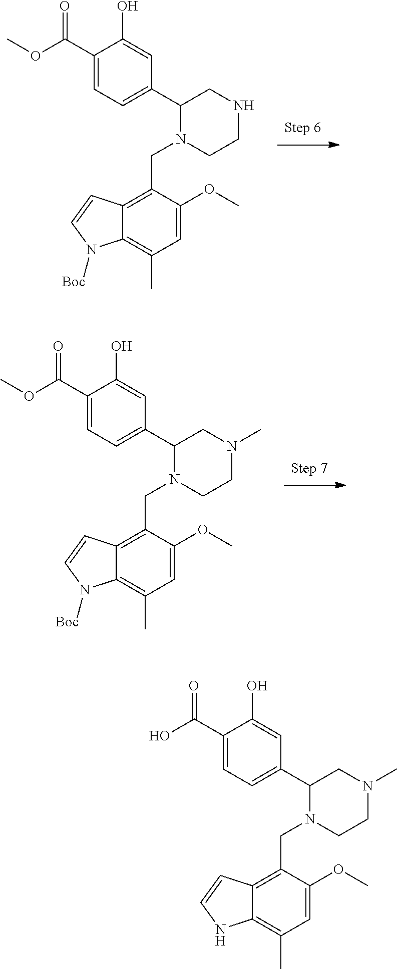

Step 1. Methyl 2-hydroxy-4-(pyrazin-2-yl)benzoate was prepared following General Experimental Procedure 1. Methyl 2-hydroxy-4-(4,4,5,5-tetramethyl-1,3,2-dioxaborolan-2-yl)benzoate (6.43 g, 17.6 mmol) and 2-chloropyrazine (2.1 g, 18.0 mmol) gave methyl 2-hydroxy-4-(pyrazin-2-yl)benzoate (2.20 g, 55%) as a yellow solid. LCMS (General 3 Basic) RT: 1.35 min; area % (254 nm): 95%; m/z=522.3 [M+H]⁺.

Step 2. Methyl 2-hydroxy-4-(piperazin-2-yl)benzoate was prepared following General Experimental Procedure 2. Methyl 2-hydroxy-4-(pyrazin-2-yl)benzoate (2.20 g, 9.57 mmol) gave methyl 2-hydroxy-4-(piperazin-2-yl)benzoate (676 mg, 30%) as an orange oil. LCMS (General 3 Basic) RT: 0.56 min; area % (254 nm): 95%; m/z=237.1 [M+H]⁺.

Step 3. Benzyl 3-(3-hydroxy-4-(methoxycarbonyl)phenyl)piperazine-1-carboxylate was prepared following General Experimental Procedure 3. Methyl 2-hydroxy-4-(piperazin-2-yl)benzoate (676 mg, 2.86 mmol) gave benzyl 3-(3-hydroxy-4-(methoxycarbonyl)phenyl)piperazine-1-carboxylate (796 mg, 75%). LCMS (General 3 Basic) RT: 1.59 min; area % (254 nm): 98%; m/z=371.2 [M+H]⁺.

Step 4. tert-Butyl 4-((4-((benzyloxy)carbonyl)-2-(3-hydroxy-4-(methoxycarbonyl)phenyl)piperazin-1-yl)methyl)-5-methoxy-7-methyl-1H-indole-1-carboxylate was prepared following General Experimental Procedure 4. Benzyl 3-(3-hydroxy-4-(methoxycarbonyl)phenyl)piperazine-1-carboxylate (951 mg, 2.57 mmol) and tert-butyl 4-formyl-5-methoxy-7-methyl-1H-indole-1-carboxylate (1.11 g, 3.84 mmol) gave tert-butyl 4-((4-((benzyloxy)carbonyl)-2-(3-hydroxy-4-(methoxycarbonyl)phenyl)piperazin-1-yl)methyl)-5-methoxy-7-methyl-1H-indole-1-carboxylate (1.01 g, 61%). LCMS (General 2 Acidic) RT: 0.99 min; area % (254 nm): 94%; m/z=644.5 [M+H]⁺.

Step 5. tert-Butyl 4-((2-(3-hydroxy-4-(methoxycarbonyl)phenyl)piperazin-1-yl)methyl)-5-methoxy-7-methyl-1H-indole-1-carboxylate was prepared following General Experimental Procedure 5. tert-Butyl 4-((4-((benzyloxy)carbonyl)-2-(3-hydroxy-4-(methoxycarbonyl)phenyl)piperazin-1-yl)methyl)-5-methoxy-7-methyl-1H-indole-1-carboxylate (398 mg, 618 μmol) gave tert-butyl 4-((2-(3-hydroxy-4-(methoxycarbonyl)phenyl)piperazin-1-yl)methyl)-5-methoxy-7-methyl-1H-indole-1-carboxylate (323 mg, >100%) as a white foam. LCMS (General 2 Basic) RT: 1.75 min; area % (254 nm): 99%; m/z=510.2 [M+H]⁺.

Step 6. tert-Butyl 4-((2-(3-hydroxy-4-(methoxycarbonyl)phenyl)-4-methylpiperazin-1-yl)methyl)-5-methoxy-7-methyl-1H-indole-1-carboxylate was prepared following General Experimental Procedure 6. tert-Butyl 4-((2-(3-hydroxy-4-(methoxycarbonyl)phenyl)piperazin-1-yl)methyl)-5-methoxy-7-methyl-1H-indole-1-carboxylate (133 mg, 262 μmol) and 37 wt. % aq. formaldehyde (117 μL, 1.57 mmol) gave crude tert-butyl 4-((2-(3-hydroxy-4-(methoxycarbonyl)phenyl)-4-methylpiperazin-1-yl)methyl)-5-methoxy-7-methyl-1H-indole-1-carboxylate (129 mg) as an amber oil. LCMS (General 2 Basic) RT: 2.04 min; area % (254 nm): 97%; m/z=524.2 [M+H]⁺.

Step 7. 2-Hydroxy-4-(1-((5-methoxy-7-methyl-1H-indol-4-yl)methyl)-4-methylpiperazin-2-yl)benzoic acid was prepared following General Experimental Procedure 8. Crude tert-butyl 4-((2-(3-hydroxy-4-(methoxycarbonyl)phenyl)-4-methylpiperazin-1-yl)methyl)-5-methoxy-7-methyl-1H-indole-1-carboxylate (129 mg) gave 2-hydroxy-4-(1-((5-methoxy-7-methyl-1H-indol-4-yl)methyl)-4-methylpiperazin-2-yl)benzoic acid (50 mg, 50% over two steps) as a white solid. LCMS (31697 LCMS-5 C3) RT: 1.471 min; area % (215 nm): 95.7%; m/z=410.0 [M+H]⁺. ¹H-NMR (400 MHz, CD₃OD) δ 7.96 (d, J=7.9 Hz, 1H), 7.25 (d, J=3.1 Hz, 1H), 7.12 (s, 1H), 7.02 (d, J=7.8 Hz, 1H), 6.72 (s, 1H), 6.41 (d, J=3.1 Hz, 1H), 4.08 (s, 1H), 3.80 (s, 3H), 3.75-3.52 (m, 1H), 3.24 (d, J=12.5 Hz, 1H), 3.19-3.08 (m, 2H), 2.86 (s, 1H), 2.76 (s, 1H), 2.61 (s, 3H), 2.51-2.44 (m, 3H).

### Example 63. 2-Fluoro-4-(1-((5-methoxy-7-methyl-1H-indol-4-yl)methyl)-4-methylpiperazin-2-yl)benzamide

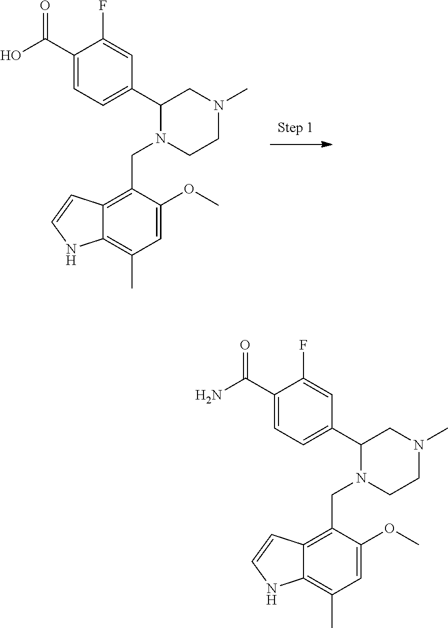 [SMILES: COc1cc(C)c2[nH]ccc2c1CN1CCN(C)CC1c1ccc(C(=O)O)c(F)c1 ; COc1cc(C)c2[nH]ccc2c1CN1CCN(C)CC1c1ccc(C(N)=O)c(F)c1]

Step 1. 2-Fluoro-4-(1-((5-methoxy-7-methyl-1H-indol-4-yl)methyl)-4-methylpiperazin-2-yl)benzamide was prepared according to the following procedure. To a solution of 2-fluoro-4-(1-((5-methoxy-7-methyl-1H-indol-4-yl)methyl)-4-methylpiperazin-2-yl)benzoic acid (23 mg, 1.0 eq, 56 μmol) in DMF (2.0 mL), Et₃N (23 μL, 3.0 eq, 0.17 mmol) and NH₄HCO₃ (44 mg, 10 eq, 0.56 mmol) were added followed by 50 wt. % T3P in DMF (50 μL, 1.5 eq, 86 μmol). The reulting mixture was stirred at 20° C. for 5 h. LCMS indicated incomplete conversion. Continued to stir at 20° C. for 10 h. LCMS indicated more conversion, but conversion was incomplete. Additional Et₃N (30 μL), NH₄HCO₃ (40 mg) and 50 wt. % T3P in DMF (50 μL) were added and the mixture was heated to 50° C. for 2 days. LCMS indicated mainly phosphonic anhydride intermediate and desired product, NH₄HCO₃ (40 mg) was added and the mixture left to stir for 3 days. The crude reaction mixture was purified by automated reverse phase FCC to afford 2-fluoro-4-(1-((5-methoxy-7-methyl-1H-indol-4-yl)methyl)-4-methylpiperazin-2-yl)benzamide (8.0 mg, 35%) as a white solid. LCMS (31697 LCMS-5 C3) RT: 1.858 min; area % (215 nm): 98.6%; m/z=412.2 [M+H]⁺. ¹H-NMR (400 MHz, CD₃OD) δ 7.86 (t, J=7.9 Hz, 1H), 7.54 (d, J=12.4 Hz, 1H), 7.47 (d, J=7.9 Hz, 1H), 7.21 (d, J=3.1 Hz, 1H), 6.69 (s, 1H), 6.45 (d, J=3.1 Hz, 1H), 3.82 (d, J=11.9 Hz, 1H), 3.79 (s, 3H), 3.49 (dd, J=10.7, 3.0 Hz, 1H), 2.85 (dt, J=12.0, 2.7 Hz, 1H), 2.79 (dt, J=11.4, 2.7 Hz, 1H), 2.73 (dq, J=11.6, 2.5 Hz, 1H), 2.46 (d, J=0.8 Hz, 3H), 2.40 (td, J=12.0, 2.8 Hz, 1H), 2.25 (s, 3H), 2.21-2.08 (m, 2H). ¹⁹F-NMR (CD₃OD) δ −115.08.

### Example 64. 2-Fluoro-4-(1-((5-methoxy-7-methyl-1H-indol-4-yl)methyl)-4-(2,2,2-trifluoroethyl)piperazin-2-yl)benzoic acid

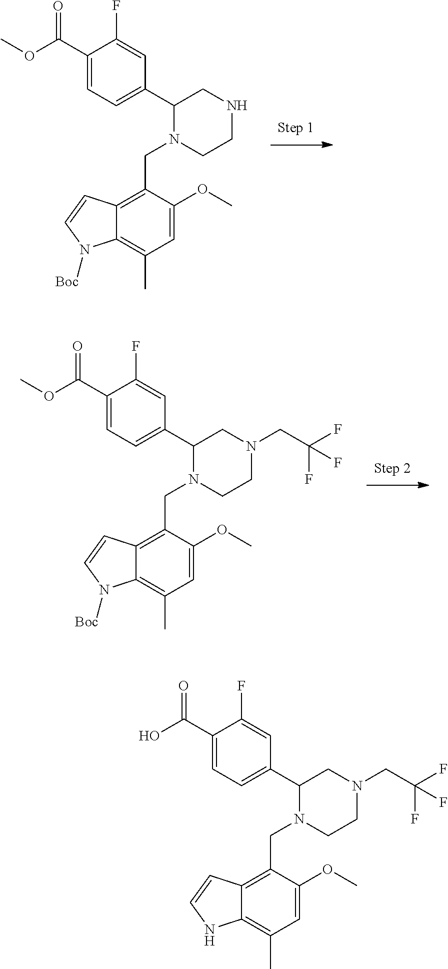

Step 1. tert-Butyl 4-((2-(3-fluoro-4-(methoxycarbonyl)phenyl)-4-(2,2,2-trifluoroethyl)piperazin-1-yl)methyl)-5-methoxy-7-methyl-1H-indole-1-carboxylate was prepared following General Experimental Procedure 7. tert-Butyl 4-((2-(3-fluoro-4-(methoxycarbonyl)phenyl)piperazin-1-yl)methyl)-5-methoxy-7-methyl-1H-indole-1-carboxylate (51 mg, 0.10 mmol) and 2,2,2-trifluoroethyl trifluoromethanesulfonate (43 μL, 0.30 mmol) gave crude tert-butyl 4-((2-(3-fluoro-4-(methoxycarbonyl)phenyl)-4-(2,2,2-trifluoroethyl)piperazin-1-yl)methyl)-5-methoxy-7-methyl-1H-indole-1-carboxylate (59 mg). LCMS (General 2 Basic) RT: 2.24 min; area % (254 nm): 84%; m/z=594.3 [M+H]⁺.

Step 2. 2-Fluoro-4-(1-((5-methoxy-7-methyl-1H-indol-4-yl)methyl)-4-(2,2,2-trifluoroethyl)piperazin-2-yl)benzoic acid was prepared following General Experimental Procedure 8. Crude tert-butyl 4-((2-(3-fluoro-4-(methoxycarbonyl)phenyl)-4-(2,2,2-trifluoroethyl)piperazin-1-yl)methyl)-5-methoxy-7-methyl-1H-indole-1-carboxylate (59 mg) gave 2-fluoro-4-(1-((5-methoxy-7-methyl-1H-indol-4-yl)methyl)-4-(2,2,2-trifluoroethyl)piperazin-2-yl)benzoic acid (41.2 mg, 86% over two steps) as a white solid. LCMS (31697 LCMS-5 C3) RT: 2.080 min; area % (215 nm): 97.8%; m/z=480.2 [M+H]⁺. ¹H-NMR (400 MHz, CD₃OD) δ 7.83 (t, J=7.6 Hz, 1H), 7.43 (d, J=11.3 Hz, 1H), 7.39 (dd, J=7.9, 1.6 Hz, 1H), 7.27 (d, J=3.1 Hz, 1H), 6.74 (s, 1H), 6.42 (d, J=3.2 Hz, 1H), 4.13 (d, J=12.5 Hz, 2H), 3.80 (s, 3H), 3.73 (d, J=31.5 Hz, 1H), 3.23-3.10 (m, 3H), 3.06 (d, J=12.0 Hz, 1H), 2.98 (d, J=11.9 Hz, 2H), 2.70 (dt, J=26.6, 7.4 Hz, 2H), 2.54-2.43 (m, 3H). ¹⁹F-NMR (CD₃OD) δ −71.07, −113.58.

### Example 64. 4-(1-((5-Methoxy-7-methyl-1H-indol-4-yl)methyl)-4-(2,2,2-trifluoroethyl)piperazin-2-yl)-2-methylbenzoic acid

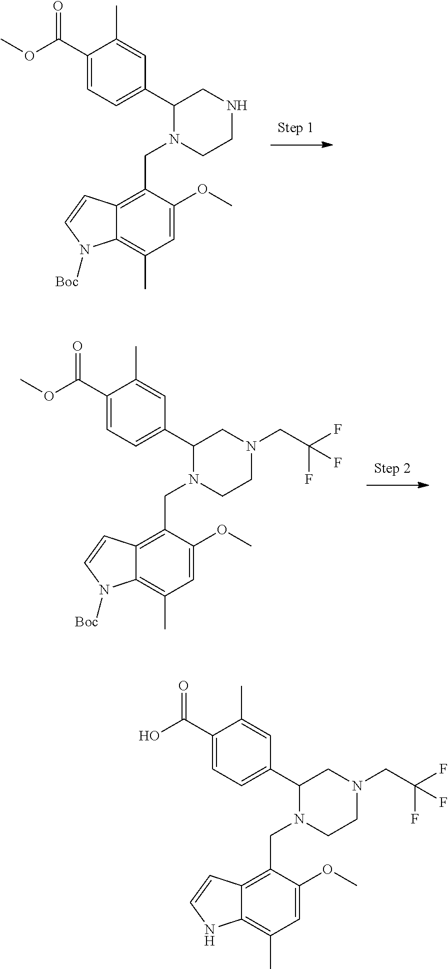

Step 1. tert-Butyl 5-methoxy-4-((2-(4-(methoxycarbonyl)-3-methylphenyl)-4-(2,2,2-trifluoroethyl)piperazin-1-yl)methyl)-7-methyl-1H-indole-1-carboxylate was prepared following General Experimental Procedure 7. tert-Butyl 5-methoxy-4-((2-(4-(methoxycarbonyl)-3-methylphenyl)piperazin-1-yl)methyl)-7-methyl-1H-indole-1-carboxylate (50 mg, 98 μmol) and 2,2,2-trifluoroethyl trifluoromethanesulfonate (43 μL, 0.30 mmol) gave crude tert-butyl 5-methoxy-4-((2-(4-(methoxycarbonyl)-3-methylphenyl)-4-(2,2,2-trifluoroethyl)piperazin-1-yl)methyl)-7-methyl-1H-indole-1-carboxylate (58 mg). LCMS (General 2 Basic) RT: 2.36 min; area % (254 nm): 91%; m/z=590.3 [M+H]⁺.

Step 2. 4-(1-((5-Methoxy-7-methyl-1H-indol-4-yl)methyl)-4-(2,2,2-trifluoroethyl)piperazin-2-yl)-2-methylbenzoic acid was prepared following General Experimental Procedure 8. Crude tert-butyl 5-methoxy-4-((2-(4-(methoxycarbonyl)-3-methylphenyl)-4-(2,2,2-trifluoroethyl)piperazin-1-yl)methyl)-7-methyl-1H-indole-1-carboxylate (58 mg) gave 4-(1-((5-methoxy-7-methyl-1H-indol-4-yl)methyl)-4-(2,2,2-trifluoroethyl)piperazin-2-yl)-2-methylbenzoic acid (28.4 mg, 61% over two steps) as a white solid. LCMS (31697 LCMS-5 C3) RT: 2.089 min; area % (215 nm): 95.8%; m/z=476.2 [M+H]⁺. ¹H-NMR (400 MHz, CD₃OD) δ 7.72 (d, J=7.9 Hz, 1H), 7.44 (s, 1H), 7.41 (d, J=8.5 Hz, 1H), 7.27 (d, J=3.1 Hz, 1H), 6.74 (s, 1H), 6.37 (d, J=3.1 Hz, 1H), 4.17 (d, J=12.4 Hz, 2H), 3.83 (d, J=13.3 Hz, 1H), 3.78 (s, 3H), 3.16 (tt, J=9.5, 4.4 Hz, 4H), 3.03 (t, J=15.0 Hz, 3H), 2.86 (d, J=11.0 Hz, 1H), 2.77 (d, J=12.3 Hz, 1H), 2.60 (s, 3H), 2.49 (d, J=0.8 Hz, 3H). ¹⁹F-NMR (CD₃OD) δ −71.05.

### Example 64. 2-Methoxy-4-(1-((5-methoxy-7-methyl-1H-indol-4-yl)methyl)-4-methylpiperazin-2-yl)benzoic acid

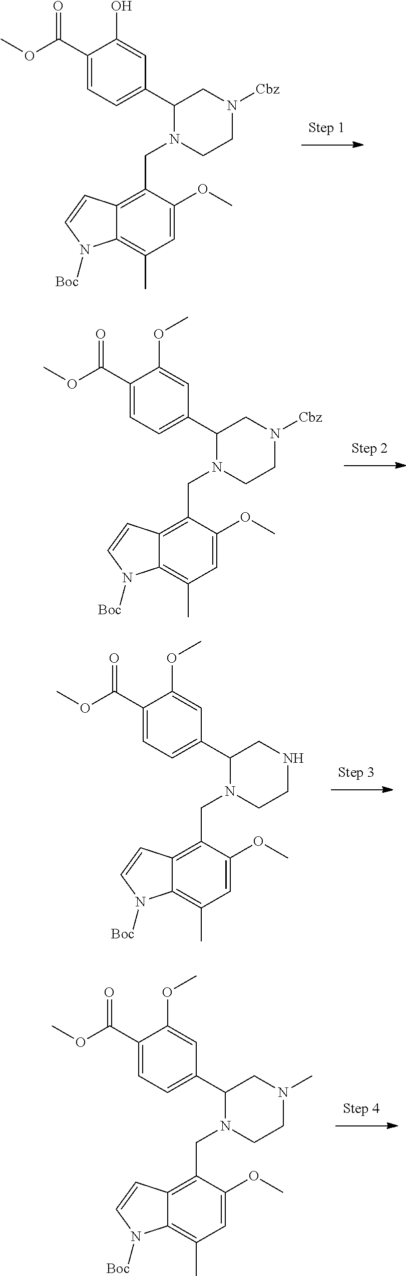

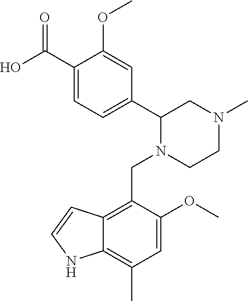 [SMILES: COc1cc(C2CN(C)CCN2Cc2c(OC)cc(C)c3[nH]ccc23)ccc1C(=O)O]

Step 1. tert-Butyl 4-((4-((benzyloxy)carbonyl)-2-(3-methoxy-4-(methoxycarbonyl)phenyl)piperazin-1-yl)methyl)-5-methoxy-7-methyl-1H-indole-1-carboxylate was prepared according to the following procedure. To a solution of tert-butyl 4-((4-((benzyloxy)carbonyl)-2-(3-hydroxy-4-(methoxycarbonyl)phenyl)piperazin-1-yl)methyl)-5-methoxy-7-methyl-1H-indole-1-carboxylate (150 mg, 1.0 eq, 233 μmol) in DMF (5.0 mL) were added K₂CO₃ (50 mg, 1.6 eq, 0.36 mmol) and CH₃I (22 μL, 1.5 eq, 0.35 mmol). The resulting suspension was stirred at 20° C. for 15 h. LCMS indicated incomplete conversion. Additional K₂CO₃ (58 mg) and CH₃I (25 μL) were added and the mixture stirred at 20° C. for 3 h. The reaction was worked-up; filtered over Celite® and the filtrate concentrated under reduced pressure to afford crude tert-butyl 4-((4-((benzyloxy)carbonyl)-2-(3-methoxy-4-(methoxycarbonyl)phenyl)piperazin-1-yl)methyl)-5-methoxy-7-methyl-1H-indole-1-carboxylate (158 mg). LCMS (General 2 Basic) RT: 2.17 min; area % (254 nm): 95%; m/z=658.4 [M+H]⁺.

Step 2. tert-Butyl 5-methoxy-4-((2-(3-methoxy-4-(methoxycarbonyl)phenyl)piperazin-1-yl)methyl)-7-methyl-1H-indole-1-carboxylate was prepared following General Experimental Procedure 5. Crude tert-butyl 4-((4-((benzyloxy)carbonyl)-2-(3-methoxy-4-(methoxycarbonyl)phenyl)piperazin-1-yl)methyl)-5-methoxy-7-methyl-1H-indole-1-carboxylate (158 mg) gave crude tert-butyl 5-methoxy-4-((2-(3-methoxy-4-(methoxycarbonyl)phenyl)piperazin-1-yl)methyl)-7-methyl-1H-indole-1-carboxylate (210 mg). LCMS (General 2 Basic) RT: 1.48 min; area % (254 nm): 92%; m/z=524.2 [M+H]⁺.

Step 3. tert-Butyl 5-methoxy-4-((2-(3-methoxy-4-(methoxycarbonyl)phenyl)-4-methylpiperazin-1-yl)methyl)-7-methyl-1H-indole-1-carboxylate was prepared following General Experimental Procedure 6. Crude tert-butyl 5-methoxy-4-((2-(3-methoxy-4-(methoxycarbonyl)phenyl)piperazin-1-yl)methyl)-7-methyl-1H-indole-1-carboxylate (210 mg) and 37 wt. % aq. formaldehyde (136 μL, 1.81 mmol) gave crude tert-butyl 5-methoxy-4-((2-(3-methoxy-4-(methoxycarbonyl)phenyl)-4-methylpiperazin-1-yl)methyl)-7-methyl-1H-indole-1-carboxylate (58 mg). LCMS (General 2 Basic) RT: 1.74 min; area % (254 nm): 100%; m/z=538.3 [M+H]⁺.

Step 4. 2-Methoxy-4-(1-((5-methoxy-7-methyl-1H-indol-4-yl)methyl)-4-methylpiperazin-2-yl)benzoic acid was prepared following General Experimental Procedure 8. Crude tert-butyl 5-methoxy-4-((2-(3-methoxy-4-(methoxycarbonyl)phenyl)-4-methylpiperazin-1-yl)methyl)-7-methyl-1H-indole-1-carboxylate (58 mg) gave 2-methoxy-4-(1-((5-methoxy-7-methyl-1H-indol-4-yl)methyl)-4-methylpiperazin-2-yl)benzoic (33 mg, 33% over four steps) as a white solid. LCMS (31697 LCMS-5 C3) RT: 1.329 min; area % (215 nm): 98.5%; m/z=424.2 [M+H]⁺. ¹H-NMR (400 MHz, CD₃OD) δ 7.61 (d, J=7.7 Hz, 1H), 7.28 (s, 1H), 7.23 (d, J=3.1 Hz, 1H), 7.17 (d, J=7.0 Hz, 1H), 6.71 (s, 1H), 6.43 (d, J=3.1 Hz, 1H), 3.96 (d, J=14.4 Hz, 1H), 3.94 (s, 3H), 3.77 (s, 3H), 3.56 (d, J=12.1 Hz, 1H), 3.14 (dt, J=12.2, 2.5 Hz, 1H), 3.08 (t, J=9.0 Hz, 2H), 2.78 (t, J=11.7 Hz, 1H), 2.73-2.64 (m, 2H), 2.56 (s, 3H), 2.47 (s, 3H).

### Example 65. 2-Methoxy-4-(1-((5-methoxy-7-methyl-1H-indol-4-yl)methyl)-4-(2,2,2-trifluoroethyl)piperazin-2-yl)benzoic acid

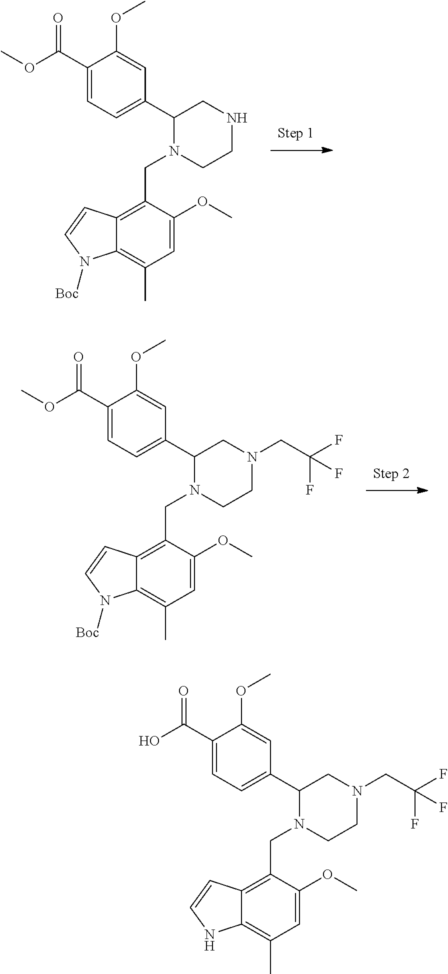

Step 1. tert-Butyl 5-methoxy-4-((2-(3-methoxy-4-(methoxycarbonyl)phenyl)-4-(2,2,2-trifluoroethyl)piperazin-1-yl)methyl)-7-methyl-1H-indole-1-carboxylate was prepared following General Experimental Procedure 7. tert-Butyl 5-methoxy-4-((2-(3-methoxy-4-(methoxycarbonyl)phenyl)piperazin-1-yl)methyl)-7-methyl-1H-indole-1-carboxylate (52 mg, 99 μmol) and 2,2,2-trifluoroethyl trifluoromethanesulfonate (43 μL, 0.30 mmol) gave crude tert-butyl 5-methoxy-4-((2-(3-methoxy-4-(methoxycarbonyl)phenyl)-4-(2,2,2-trifluoroethyl)piperazin-1-yl)methyl)-7-methyl-1H-indole-1-carboxylate (82 mg). LCMS (General 2 Basic) RT: 2.12 min; area % (254 nm): 80%; m/z=606.3 [M+H]⁺.

Step 2. 2-Methoxy-4-(1-((5-methoxy-7-methyl-1H-indol-4-yl)methyl)-4-(2,2,2-trifluoroethyl)piperazin-2-yl)benzoic acid was prepared following General Experimental Procedure 8. Crude tert-butyl 5-methoxy-4-((2-(3-methoxy-4-(methoxycarbonyl)phenyl)-4-(2,2,2-trifluoroethyl)piperazin-1-yl)methyl)-7-methyl-1H-indole-1-carboxylate (82 mg) gave 2-methoxy-4-(1-((5-methoxy-7-methyl-1H-indol-4-yl)methyl)-4-(2,2,2-trifluoroethyl)piperazin-2-yl)benzoic acid (30.0 mg, 62% over two steps) as a white solid. LCMS (31697 LCMS-5 C3) RT: 2.061 min; area % (215 nm): 94.2%; m/z=492.2 [M+H]⁺. ¹H-NMR (400 MHz, CD₃OD) δ 7.59 (d, J=7.7 Hz, 1H), 7.26 (s, 1H), 7.23 (d, J=3.1 Hz, 1H), 7.15 (d, J=7.5 Hz, 1H), 6.71 (s, 1H), 6.44 (dd, J=3.2, 1.4 Hz, 1H), 4.02 (d, J=11.1 Hz, 1H), 3.92 (s, 3H), 3.77 (s, 4H), 3.59 (s, 1H), 3.16-3.06 (m, 2H), 2.97 (dt, J=22.3, 11.8 Hz, 3H), 2.81-2.59 (m, 4H), 2.47 (s, 3H).

### Example 66. 2-Hydroxy-4-(1-((5-methoxy-7-methyl-1H-indol-4-yl)methyl)-4-(2,2,2-trifluoroethyl)piperazin-2-yl)benzoic acid

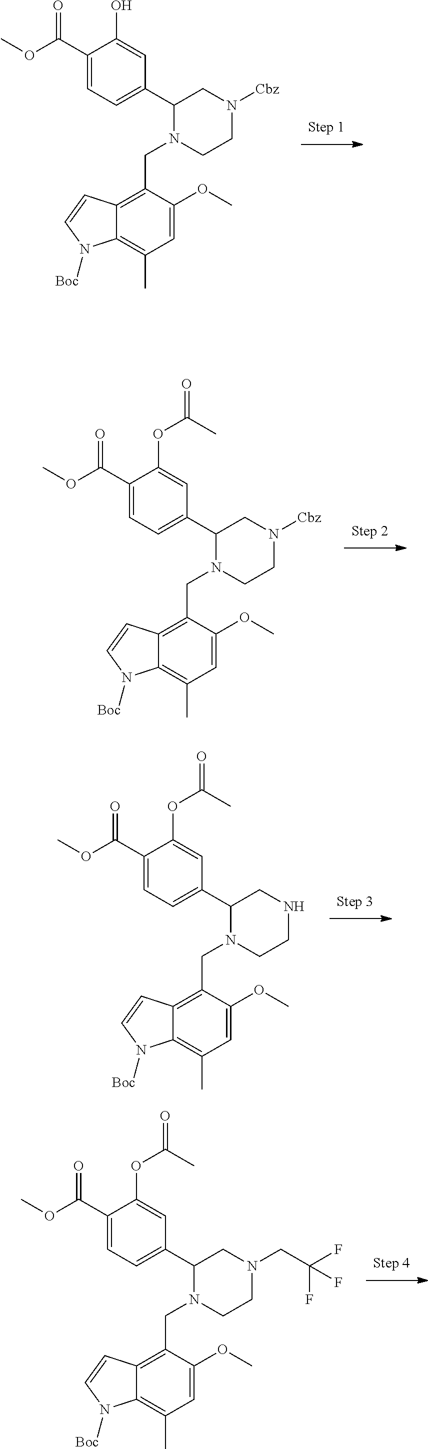

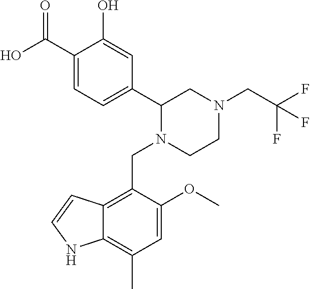 [SMILES: COc1cc(C)c2[nH]ccc2c1CN1CCN(CC(F)(F)F)CC1c1ccc(C(=O)O)c(O)c1]

Step 1. tert-Butyl 4-((2-(3-acetoxy-4-(methoxycarbonyl)phenyl)-4-((benzyloxy)carbonyl)piperazin-1-yl)methyl)-5-methoxy-7-methyl-1H-indole-1-carboxylate was prepared according to the following procedure. To a solution tert-butyl 4-((4-((benzyloxy)carbonyl)-2-(3-hydroxy-4-(methoxycarbonyl)phenyl)piperazin-1-yl)methyl)-5-methoxy-7-methyl-1H-indole-1-carboxylate (183 mg, 1.0 eq, 284 μmol) in DCM (5 mL) were added Ac₂O (30 μL, 1.1 eq, 0.32 mmol) and Et₃N (48 μL, 1.2 eq, 0.34 mmol). The resulting solution was stirred at 20° C. for 15 h. TLC indicated incomplete conversion. Additional Ac₂O (260 μL) and Et₃N (390 μL) were added and stirring continued at 20° C. for 15 h. The reaction was worked-up; concentrated under reduced pressure and the residue partitioned between EtOAc (20 mL) and sat. aq. NaHCO₃ (20 mL). The layers were separated and the organic layer washed with brine (20 mL), dried over Na₂SO₄, filtered, and concentrated under reduced pressure to afford crude tert-butyl 4-((2-(3-acetoxy-4-(methoxycarbonyl)phenyl)-4-((benzyloxy)carbonyl)piperazin-1-yl)methyl)-5-methoxy-7-methyl-1H-indole-1-carboxylate (215 mg) as a yellow oil. LCMS (General 2 Basic) RT: 2.18 min; area % (254 nm): 100%; m/z=686.3 [M+H]⁺.

Step 2. tert-Butyl 4-((2-(3-acetoxy-4-(methoxycarbonyl)phenyl)piperazin-1-yl)methyl)-5-methoxy-7-methyl-1H-indole-1-carboxylate was prepared following General Experimental Procedure 5. Crude tert-butyl 4-((2-(3-acetoxy-4-(methoxycarbonyl)phenyl)-4-((benzyloxy)carbonyl)piperazin-1-yl)methyl)-5-methoxy-7-methyl-1H-indole-1-carboxylate (215 mg) gave crude tert-butyl 4-((2-(3-acetoxy-4-(methoxycarbonyl)phenyl)piperazin-1-yl)methyl)-5-methoxy-7-methyl-1H-indole-1-carboxylate (241 mg). LCMS (General 2 Basic) RT: 1.54 min; area % (254 nm): 87%; m/z=552.3 [M+H]⁺.

Step 3. tert-Butyl 4-((2-(3-acetoxy-4-(methoxycarbonyl)phenyl)-4-(2,2,2-trifluoroethyl)piperazin-1-yl)methyl)-5-methoxy-7-methyl-1H-indole-1-carboxylate was prepared following General Experimental Procedure 7. Crude tert-butyl 4-((2-(3-acetoxy-4-(methoxycarbonyl)phenyl)piperazin-1-yl)methyl)-5-methoxy-7-methyl-1H-indole-1-carboxylate (84 mg) and 2,2,2-trifluoroethyl trifluoromethylsulfonate (85 μL, 0.61 mmol) gave crude tert-butyl 4-((2-(3-acetoxy-4-(methoxycarbonyl)phenyl)-4-(2,2,2-trifluoroethyl)piperazin-1-yl)methyl)-5-methoxy-7-methyl-1H-indole-1-carboxylate (142 mg). LCMS (General 2 Basic) RT: 2.15 min; area % (254 nm): 75%; m/z=634.3 [M+H]⁺.

Step 4. 2-Hydroxy-4-(1-((5-methoxy-7-methyl-1H-indol-4-yl)methyl)-4-(2,2,2-trifluoroethyl)piperazin-2-yl)benzoic acid was prepared following General Experimental Procedure 8. Crude tert-butyl 4-((2-(3-acetoxy-4-(methoxycarbonyl)phenyl)-4-(2,2,2-trifluoroethyl)piperazin-1-yl)methyl)-5-methoxy-7-methyl-1H-indole-1-carboxylate (142 mg) gave 2-hydroxy-4-(1-((5-methoxy-7-methyl-1H-indol-4-yl)methyl)-4-(2,2,2-trifluoroethyl)piperazin-2-yl)benzoic acid (28.1 mg, 32% over four steps) as a white solid. LCMS (31697 LCMS-5 C3) RT: 2.115 min; area % (215 nm): >95.0%; m/z=478.2 [M+H]⁺. ¹H-NMR (400 MHz, CD₃OD) δ 8.00 (d, J=8.0 Hz, 1H), 7.31 (d, J=3.1 Hz, 1H), 7.10 (d, J=1.7 Hz, 1H), 7.00 (dd, J=8.0, 1.8 Hz, 1H), 6.76 (s, 1H), 6.37 (d, J=3.2 Hz, 1H), 4.39 (d, J=10.7 Hz, 2H), 4.08 (s, 1H), 3.81 (s, 3H), 3.33 (d, J=1.8 Hz, 2H), 3.27-3.14 (m, 3H), 3.08 (d, J=12.7 Hz, 1H), 2.99 (s, 1H), 2.86 (s, 1H), 2.50 (d, J=0.7 Hz, 3H).

### Example 66. 4-(4-(2,2-Difluoroethyl)-1-((5-methoxy-7-methyl-1H-indol-4-yl)methyl)piperazin-2-yl)-2-hydroxybenzoic acid

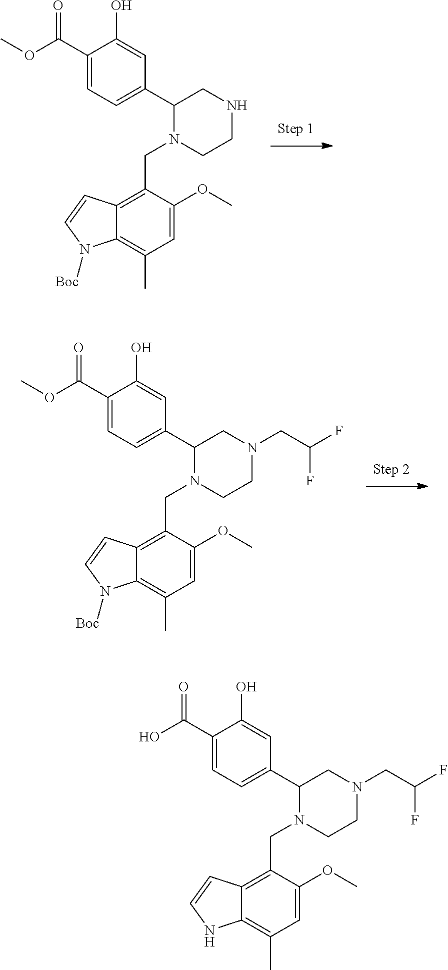

Step 1. tert-Butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate was prepared following General Experimental Procedure 7. tert-butyl 4-((2-(3-hydroxy-4-(methoxycarbonyl)phenyl)piperazin-1-yl)methyl)-5-methoxy-7-methyl-1H-indole-1-carboxylate (20 mg, 39 μmol) and 1-ethoxy-2,2-difluoroethan-1-ol (26, 0.20 mmol) gave crude tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (23 mg). LCMS (General 3 Basic) RT: 2.38 min; area % (254 nm): 81%; m/z=574.3 [M+H]⁺.

Step 2. 4-(4-(2,2-Difluoroethyl)-1-((5-methoxy-7-methyl-1H-indol-4-yl)methyl)piperazin-2-yl)-2-hydroxybenzoic acid was prepared following General Experimental Procedure 8. Crude tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (23 mg) gave 4-(4-(2,2-difluoroethyl)-1-((5-methoxy-7-methyl-1H-indol-4-yl)methyl)piperazin-2-yl)-2-hydroxybenzoic acid (3.7 mg, 20% over two steps) as a white solid. LCMS (31697 LCMS-5 C3) RT: 1.896 min; area % (215 nm): 98.3%; m/z=460.2 [M+H]⁺. ¹H-NMR (400 MHz, CD₃OD) δ 8.00 (d, J=8.0 Hz, 1H), 7.31 (d, J=3.2 Hz, 1H), 7.10 (d, J=1.8 Hz, 1H), 7.00 (dd, J=7.9, 1.8 Hz, 1H), 6.76 (s, 1H), 6.36 (d, J=3.2 Hz, 1H), 5.98 (tt, J=55.8, 4.2 Hz, 1H), 4.41 (d, J=12.4 Hz, 2H), 4.10 (d, J=12.5 Hz, 1H), 3.80 (s, 3H), 3.36 (s, 1H), 3.17 (d, J=12.9 Hz, 1H), 3.10-3.02 (m, 1H), 2.87 (tdd, J=14.4, 9.2, 4.1 Hz, 3H), 2.71 (q, J=6.3 Hz, 1H), 2.50 (d, J=0.8 Hz, 3H).

### Example 67. 2-Hydroxy-4-(1-((5-methoxy-7-methyl-1H-indol-4-yl)methyl)-4-(3,3,3-trifluoropropyl)piperazin-2-yl)benzoic acid

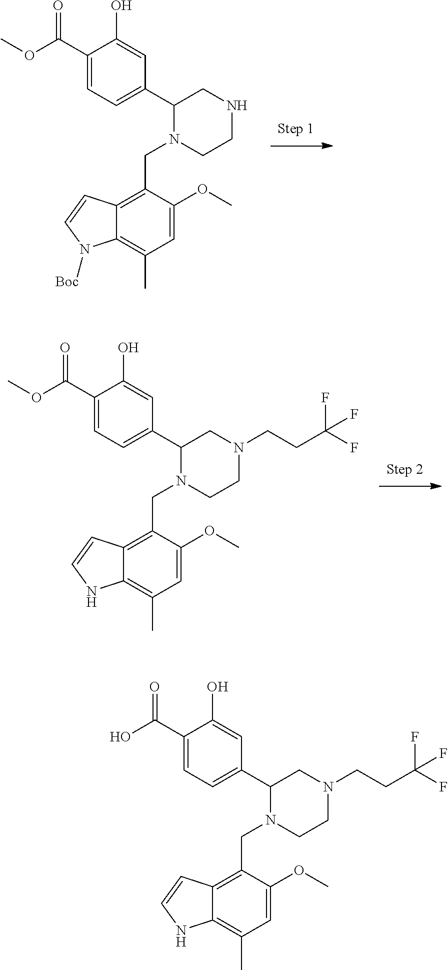

Step 1. tert-Butyl 4-((2-(3-hydroxy-4-(methoxycarbonyl)phenyl)-4-(3,3,3-trifluoropropyl)piperazin-1-yl)methyl)-5-methoxy-7-methyl-1H-indole-1-carboxylate was prepared following General Experimental Procedure 6. tert-butyl 4-((2-(3-hydroxy-4-(methoxycarbonyl)phenyl)piperazin-1-yl)methyl)-5-methoxy-7-methyl-1H-indole-1-carboxylate (43 mg, 84 μmol) and 3,3,3-trifluoropropanal (48, 0.51 mmol) gave crude tert-butyl 4-((2-(3-hydroxy-4-(methoxycarbonyl)phenyl)-4-(3,3,3-trifluoropropyl)piperazin-1-yl)methyl)-5-methoxy-7-methyl-1H-indole-1-carboxylate (25 mg). LCMS (General 3 Basic) RT: 2.46 min; area % (254 nm): 85%; m/z=606.3 [M+H]⁺.

Step 2. 2-Hydroxy-4-(1-((5-methoxy-7-methyl-1H-indol-4-yl)methyl)-4-(3,3,3-trifluoropropyl)piperazin-2-yl)benzoic acid was prepared following General Experimental Procedure 8. Crude tert-butyl 4-((2-(3-hydroxy-4-(methoxycarbonyl)phenyl)-4-(3,3,3-trifluoropropyl)piperazin-1-yl)methyl)-5-methoxy-7-methyl-1H-indole-1-carboxylate (25 mg) gave 2-hydroxy-4-(1-((5-methoxy-7-methyl-1H-indol-4-yl)methyl)-4-(3,3,3-trifluoropropyl)piperazin-2-yl)benzoic acid (12.9 mg, 31% over two steps) as a white solid. LCMS (31697 LCMS-5 C3) RT: 2.075 min; area % (215 nm): >95%; m/z=492.2 [M+H]⁺. ¹H-NMR (400 MHz, CD₃OD) δ 8.00 (d, J=7.9 Hz, 1H), 7.31 (d, J=3.2 Hz, 1H), 7.11 (d, J=1.8 Hz, 1H), 7.00 (dd, J=8.0, 1.8 Hz, 1H), 6.77 (s, 1H), 6.36 (d, J=3.2 Hz, 1H), 4.53-4.25 (m, 2H), 4.25-4.01 (m, 1H), 3.81 (s, 3H), 3.51-3.35 (m, 2H), 3.23-2.98 (m, 2H), 2.86-2.57 (m, 3H), 2.51 (s, 4H), 2.47-2.26 (m, 2H).

### Example 68. Enantiomer 1 of 4-(4-(2,2-difluoroethyl)-1-((5-methoxy-7-methyl-1H-indol-4-yl)methyl)piperazin-2-yl)-2-hydroxybenzoic acid

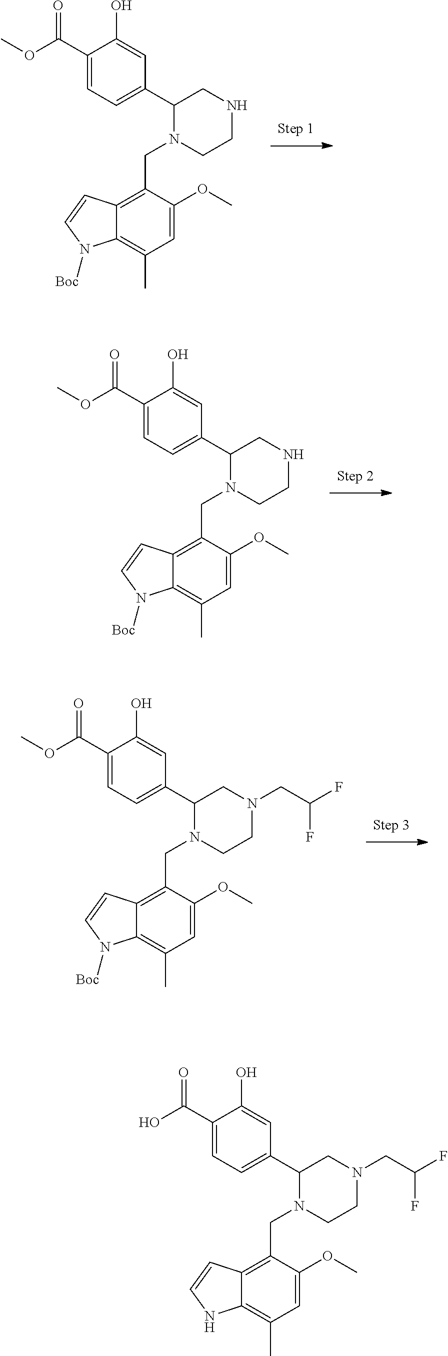

Step 1. Enantiomer 1 of tert-butyl 4-((2-(3-hydroxy-4-(methoxycarbonyl)phenyl)piperazin-1-yl)methyl)-5-methoxy-7-methyl-1H-indole-1-carboxylate was prepared according to the following procedure. Racemic tert-butyl 4-((2-(3-hydroxy-4-(methoxycarbonyl)phenyl)piperazin-1-yl)methyl)-5-methoxy-7-methyl-1H-indole-1-carboxylate was separated by chiral SFC (iCel-5 iso40) to obtain enantiomer 1 of tert-butyl 4-((2-(3-hydroxy-4-(methoxycarbonyl)phenyl)piperazin-1-yl)methyl)-5-methoxy-7-methyl-1H-indole-1-carboxylate (91 mg). First eluting enantiomer (31697 IC Gradient) RT: 3.466 min, area % (PDA): 100%; m/z=510.1 [M+H]⁺.

Step 2. Enantiomer 1 of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate was prepared according to the following procedure. Enantiomer 1 of tert-butyl 4-((2-(3-hydroxy-4-(methoxycarbonyl)phenyl)piperazin-1-yl)methyl)-5-methoxy-7-methyl-1H-indole-1-carboxylate (41 mg, 80 μmol) was converted according to Step 1 of an Example above to afford enantiomer 1 of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (22 mg, 48%) as a colorless solid. LCMS (General 3 Basic) RT: 2.39 min; area % (254 nm): 98%; m/z=574.3 [M+H]⁺.

Step 3. Enantiomer 1 of 4-(4-(2,2-difluoroethyl)-1-((5-methoxy-7-methyl-1H-indol-4-yl)methyl)piperazin-2-yl)-2-hydroxybenzoic acid was prepared with the following procedure. Enantiomer 1 of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (22 mg, 38 μmol) was converted according to Step 2 of an Example above to afford enantiomer 1 of 4-(4-(2,2-difluoroethyl)-1-((5-methoxy-7-methyl-1H-indol-4-yl)methyl)piperazin-2-yl)-2-hydroxybenzoic acid (3.6 mg, 21%). LCMS (31697 LCMS-5 C3) RT: 1.893 min; area % (215 nm): 98.8%; m/z=460.2 [M+H]⁺. ¹H-NMR identical to reported above. Based on the biological activity for this compound the first eluting enantiomer resulted in the more active enantiomer.

### Example 69. Enantiomer 2 of 4-(4-(2,2-difluoroethyl)-1-((5-methoxy-7-methyl-1H-indol-4-yl)methyl)piperazin-2-yl)-2-hydroxybenzoic acid

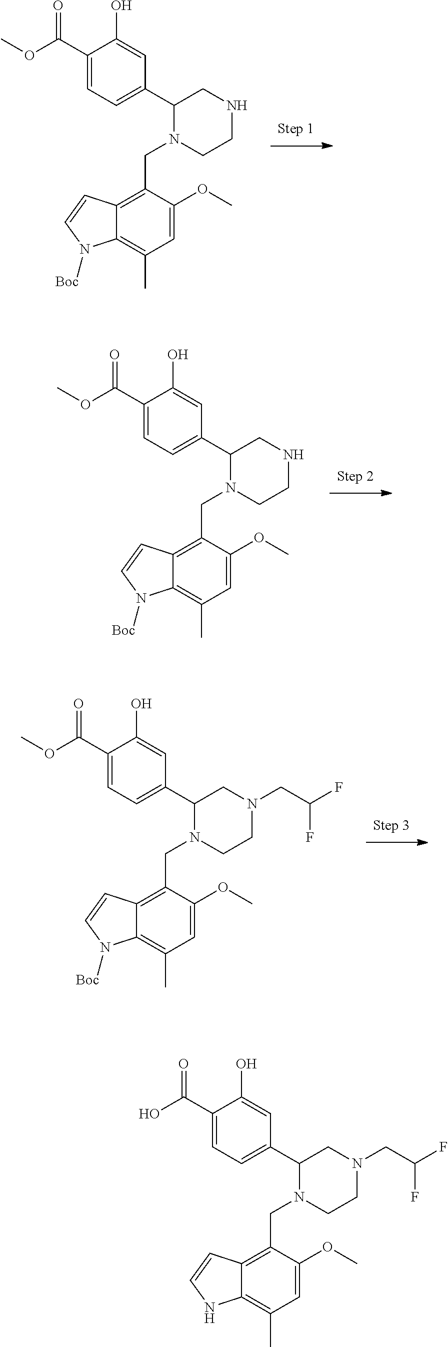

Step 1. Enantiomer 2 of tert-butyl 4-((2-(3-hydroxy-4-(methoxycarbonyl)phenyl)piperazin-1-yl)methyl)-5-methoxy-7-methyl-1H-indole-1-carboxylate was prepared according to the following procedure. Racemic tert-butyl 4-((2-(3-hydroxy-4-(methoxycarbonyl)phenyl)piperazin-1-yl)methyl)-5-methoxy-7-methyl-1H-indole-1-carboxylate was separated by chiral SFC (iCel-5 iso40) to obtained enantiomer 2 of tert-butyl 4-((2-(3-hydroxy-4-(methoxycarbonyl)phenyl)piperazin-1-yl)methyl)-5-methoxy-7-methyl-1H-indole-1-carboxylate (110 mg). Second eluting enantiomer (31697 IC Gradient) RT: 3.997 min, area % (PDA): 100%; m/z=510.1 [M+H]⁺.

Step 2. Enantiomer 2 of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate was prepared according to the following procedure. Enantiomer 2 of tert-butyl 4-((2-(3-hydroxy-4-(methoxycarbonyl)phenyl)piperazin-1-yl)methyl)-5-methoxy-7-methyl-1H-indole-1-carboxylate (40 mg, 78 μmol) was converted according to Step 1 of an Example above to afford crude enantiomer 2 of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (62 mg). LCMS (LCMS-4 General 3 AB) RT: 2.91 min; area % (254 nm): 78%; m/z=574.3 [M+H]⁺.

Step 3. Enantiomer 2 of 4-(4-(2,2-difluoroethyl)-1-((5-methoxy-7-methyl-1H-indol-4-yl)methyl)piperazin-2-yl)-2-hydroxybenzoic acid was prepared with the following procedure. Crude enantiomer 2 of tert-butyl 4-((4-(2,2-difluoroethyl)-2-(3-hydroxy-4-(methoxycarbonyl)phenyl)piperazin-1-yl)methyl)-5-methoxy-7-methyl-1H-indole-1-carboxylate (62 mg) was converted according to Step 2 of an Example above to afford enantiomer 2 of 4-(4-(2,2-difluoroethyl)-1-((5-methoxy-7-methyl-1H-indol-4-yl)methyl)piperazin-2-yl)-2-hydroxybenzoic acid (24.4 mg, 49%). LCMS (31697 LCMS-5 C3) RT: 1.893 min; area % (215 nm): >95%; m/z=460.2 [M+H]⁺. ¹H-NMR identical to reported above.

### Example 70. 2-Hydroxy-4-(1-((5-methoxy-7-methyl-1H-indol-4-yl)methyl)-4-(2,2,2-trifluoroethyl)piperazin-2-yl)benzoic acid

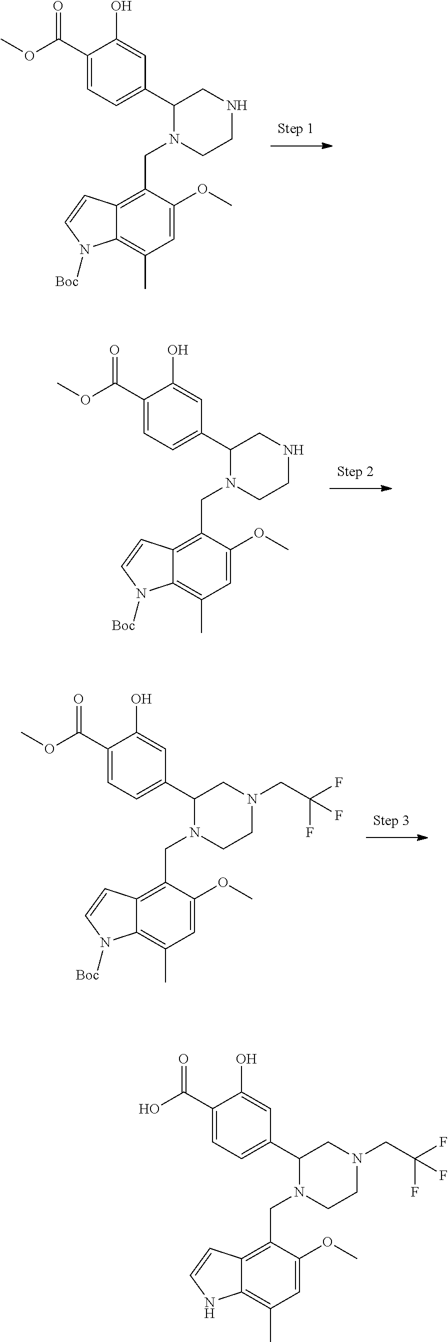

Step 1. Enantiomer 1 of tert-butyl 4-((2-(3-hydroxy-4-(methoxycarbonyl)phenyl)piperazin-1-yl)methyl)-5-methoxy-7-methyl-1H-indole-1-carboxylate was prepared according to the following procedure. Racemic tert-butyl 4-((2-(3-hydroxy-4-(methoxycarbonyl)phenyl)piperazin-1-yl)methyl)-5-methoxy-7-methyl-1H-indole-1-carboxylate was separated by chiral SFC (iCel-5 iso40) to obtain enantiomer 1 of tert-butyl 4-((2-(3-hydroxy-4-(methoxycarbonyl)phenyl)piperazin-1-yl)methyl)-5-methoxy-7-methyl-1H-indole-1-carboxylate (91 mg). First eluting enantiomer (31697 IC Gradient) RT: 3.466 min, area % (PDA): 100%; m/z=510.1 [M+H]⁺.

Step 2. Enantiomer 1 of tert-butyl 4-((2-(3-hydroxy-4-(methoxycarbonyl)phenyl)-4-(2,2,2-trifluoroethyl)piperazin-1-yl)methyl)-5-methoxy-7-methyl-1H-indole-1-carboxylate was prepared following General Experimental Procedure 7. Enantiomer 1 of tert-butyl 4-((2-(3-hydroxy-4-(methoxycarbonyl)phenyl)piperazin-1-yl)methyl)-5-methoxy-7-methyl-1H-indole-1-carboxylate (41 mg, 80 μmol) and 2,2,2-trifluoroethyl trifluoromethanesulfonate (36 μL, 0.23 mmol) to afford enantiomer 1 of tert-butyl 4-((2-(3-hydroxy-4-(methoxycarbonyl)phenyl)-4-(2,2,2-trifluoroethyl)piperazin-1-yl)methyl)-5-methoxy-7-methyl-1H-indole-1-carboxylate (37 mg, 79%) as a colorless solid. LCMS (LCMS-4 General 3 AB) RT: 2.89 min; area % (254 nm): 85%; m/z=592.2 [M+H]⁺.

Step 3. Enantiomer 1 of 2-hydroxy-4-(1-((5-methoxy-7-methyl-1H-indol-4-yl)methyl)-4-(2,2,2-trifluoroethyl)piperazin-2-yl)benzoic acid was prepared following General Experimental Procedure 8. Enantiomer 1 tert-butyl 4-((2-(3-hydroxy-4-(methoxycarbonyl)phenyl)-4-(2,2,2-trifluoroethyl)piperazin-1-yl)methyl)-5-methoxy-7-methyl-1H-indole-1-carboxylate (37 mg, 62 μmol) gave enantiomer 1 of 2-hydroxy-4-(1-((5-methoxy-7-methyl-1H-indol-4-yl)methyl)-4-(2,2,2-trifluoroethyl)piperazin-2-yl)benzoic acid (2.5 mg, 8.4%). LCMS (31697 LCMS-5 C3) RT: 2.111 min; area % (215 nm): >99%; m/z=478.2 [M+H]⁺. ¹H-NMR identical to an Example above.

### Example 71. Enantiomer 1 of 2-hydroxy-4-(4-((5-methoxy-7-methyl-1H-indol-4-yl)methyl)-1-methyl-1,4-diazepan-5-yl)benzoic acid

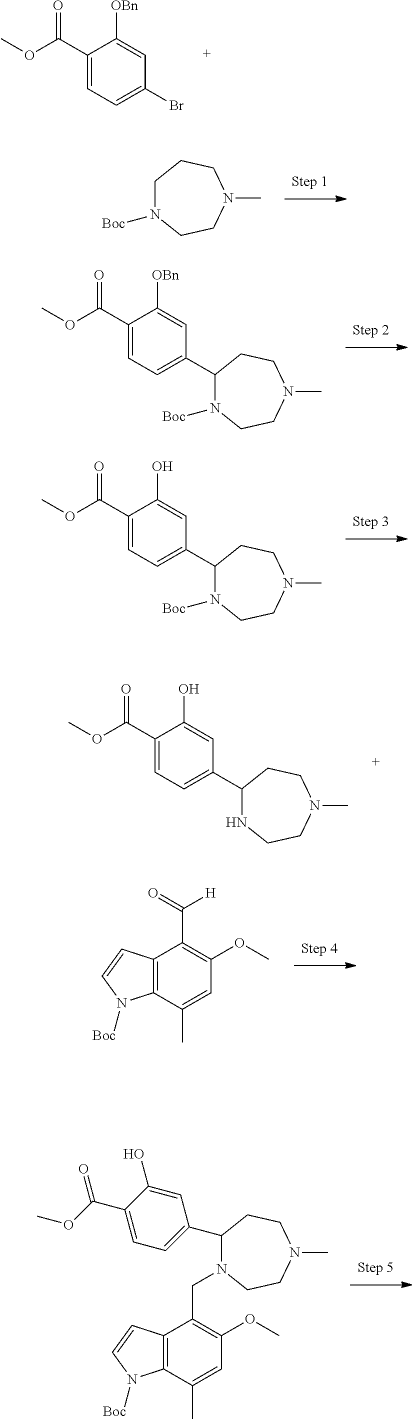

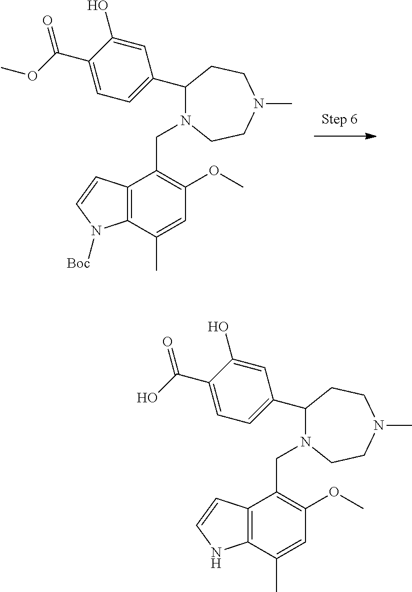

Step 1. tert-Butyl 7-(3-(benzyloxy)-4-(methoxycarbonyl)phenyl)-4-methyl-1,4-diazepane-1-carboxylate was prepared according to the following procedure. In a flame-dried round bottom flask a solution of tert-butyl 4-methyl-1,4-diazepane-1-carboxylate (801 mg, 1.5 eq, 3.74 mmol), TMEDA (598 μL, 1.6 eq, 3.99 mmol) under N₂ in dry Et₂O (25 mL) was cooled to −78° C. and 1.3 M sec-Buli in cyclohexane/hexane (3.07 mL, 1.6 eq, 3.99 mmol) was added dropwise. The reaction mixture was allowed to stir at this temperature for 2 hours before 0.7 M ZnCl₂ in THF (5.69 mL, 1.6 eq, 3.99 mmol) was added. The reaction mixture was warm to 20° C. and allowed to stir at this temperature for 2 hours. Meanwhile a solution of Pd(PPh₃)₄ (173 mg, 0.06 eq, 149 μmol) 2-dicyclohexylphosphino-2,6-dimethoxy-1,1-biphenyl (126 mg, 0.123 Eq, 306 μmol) in dry toluene (60 mL) was prepared. The catalyst solution was added to the previous prepared solution followed by methyl 2-(benzyloxy)-4-bromobenzoate (800 mg, 1 eq, 2.49 mmol). The Et₂O was removed by Dean-Stark distillation and the resulting mixture was allowed to stir at 110° C. for 15 h. The reaction was worked-up; cooled to 20° C. and quenched with saturated aqueous NH₄Cl. The mixture was extracted with EtOAc (3×). The pooled organic layers were dried over Na₂SO₄, filtered, and concentrated under reduced pressure. The residue was purified by normal phase FCC to afford tert-butyl 7-(3-(benzyloxy)-4-(methoxycarbonyl)phenyl)-4-methyl-1,4-diazepane-1-carboxylate (233 mg, 21%). LCMS (LCMS-4 General 3 AB) RT: 2.15 min; area % (254 nm): 100%; m/z=455.4 [M+H]⁺.

Step 2. tert-Butyl 7-(3-hydroxy-4-(methoxycarbonyl)phenyl)-4-methyl-1,4-diazepane-1-carboxylate was prepared according to the following procedure. A suspension of tert-butyl 7-(3-(benzyloxy)-4-(methoxycarbonyl)phenyl)-4-methyl-1,4-diazepane-1-carboxylate (233 mg, 1.0 eq, 513 μmol) and 10% Pd/C (27.3 mg, 0.5 eq, 256 μmol) in DCM (10 mL) was purged with H₂ and then stirred under H₂ pressure (balloon) at 20° C. for 15 h. The reaction was worked-up; filtered over Celite® and concentrated the filtrate under reduced pressure to afford crude tert-butyl 7-(3-hydroxy-4-(methoxycarbonyl)phenyl)-4-methyl-1,4-diazepane-1-carboxylate (187 mg). LCMS (LCMS-4 General 3 AB) RT: 1.95 min; area % (254 nm): 98%; m/z=365.4 [M+H]⁺.

Step 3. Methyl 2-hydroxy-4-(1-methyl-1,4-diazepan-5-yl)benzoate dihydrochloride was prepared according to the following procedure. To a solution of crude tert-butyl 7-(3-hydroxy-4-(methoxycarbonyl)phenyl)-4-methyl-1,4-diazepane-1-carboxylate (187 mg) in DCM (10 mL) was added 4 N HCl in dioaxane (1.28 mL, 4 molar, ~10 eq). The resulting mixture was stirred at 20° C. for 15 h. The reaction was worked-up; concentrated under reduced pressure to afford crude methyl 2-hydroxy-4-(1-methyl-1,4-diazepan-5-yl)benzoate dihydrochloride (176 mg).

Step 4. tert-Butyl 4-((7-(3-hydroxy-4-(methoxycarbonyl)phenyl)-4-methyl-1,4-diazepan-1-yl)methyl)-5-methoxy-7-methyl-1H-indole-1-carboxylate was prepared following General Experimental Procedure 4. Crude methyl 2-hydroxy-4-(1-methyl-1,4-diazepan-5-yl)benzoate dihydrochloride (176 mg) and tert-butyl 4-formyl-5-methoxy-7-methyl-1H-indole-1-carboxylate gave tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate (165 mg, 59%).). LCMS (LCMS-4 General 3 AB) RT: 2.47 min; area % (254 nm): 87%; m/z=538.4 [M+H]⁺.

Step 5. Enantiomer 1 of tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate was prepared according to the following procedure. Racemic tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate was separated by chiral SFC, first eluting enantiomer.

Step 6. Enantiomer 1 of 2-hydroxy-4-(4-((5-methoxy-7-methyl-1H-indol-4-yl)methyl)-1-methyl-1,4-diazepan-5-yl)benzoic acid was prepared following General Experimental Procedure 1. Enantiomer 1 of tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate gave enantiomer 1 of 2-hydroxy-4-(4-((5-methoxy-7-methyl-1H-indol-4-yl)methyl)-1-methyl-1,4-diazepan-5-yl)benzoic acid (28.9 mg, 54%) as a white solid. LCMS (31697 LCMS-5 C3) RT: 1.57 min; area % (215 nm): 96.2%; m/z=424.2 [M+H]⁺. ¹H-NMR (400 MHz, CD₃OD) δ 7.89 (d, J=8.0 Hz, 1H), 7.21 (d, J=3.1 Hz, 1H), 7.06 (s, 1H), 6.95 (d, J=8.3 Hz, 2H), 6.72 (s, 1H), 6.30 (s, 1H), 4.06 (s, 1H), 3.96 (s, 2H), 3.79 (s, 3H), 3.48 (p, J=1.7 Hz, 1H), 3.40-3.35 (m, 2H), 3.15 (s, 4H), 3.08-2.99 (m, 2H), 2.73 (s, 3H), 2.47 (d, J=0.8 Hz, 3H), 2.28-2.17 (m, 2H).

### Example 72. Enantiomer 2 of 2-hydroxy-4-(4-((5-methoxy-7-methyl-1H-indol-4-yl)methyl)-1-methyl-1,4-diazepan-5-yl)benzoic acid

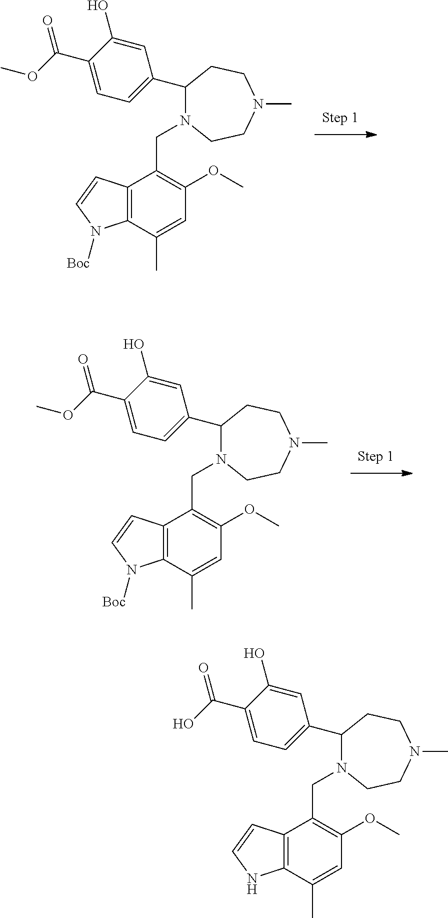

Step 1. Enantiomer 2 of tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate was prepared according to the following procedure. Racemic tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate was separated by chiral SFC, second eluting peak.

Step 2. Enantiomer 2 of 2-hydroxy-4-(4-((5-methoxy-7-methyl-1H-indol-4-yl)methyl)-1-methyl-1,4-diazepan-5-yl)benzoic acid was prepared following General Experimental Procedure 1. Enantiomer 2 of tert-butyl 4-((7-(3-hydroxy-4-(methoxycarbonyl)phenyl)-4-methyl-1,4-diazepan-1-yl)methyl)-5-methoxy-7-methyl-1H-indole-1-carboxylate gave enantiomer 2 of 2-hydroxy-4-(4-((5-methoxy-7-methyl-1H-indol-4-yl)methyl)-1-methyl-1,4-diazepan-5-yl)benzoic acid (7.5 mg, 15%) as a white solid. LCMS (31697 LCMS-5 C3) RT: 1.57 min; area % (215 nm): 95.9%; m/z=424.2 [M+H^{]+}. ¹H-NMR (400 MHz, CD₃OD) δ 7.89 (d, J=8.0 Hz, 1H), 7.21 (d, J=3.1 Hz, 1H), 7.06 (s, 1H), 6.95 (d, J=8.3 Hz, 2H), 6.72 (s, 1H), 6.30 (s, 1H), 4.06 (s, 1H), 3.96 (s, 2H), 3.79 (s, 3H), 3.48 (p, J=1.7 Hz, 1H), 3.40-3.35 (m, 2H), 3.15 (s, 4H), 3.08-2.99 (m, 2H), 2.73 (s, 3H), 2.47 (d, J=0.8 Hz, 3H), 2.28-2.17 (m, 2H).

### Example 73. 4-(4-(2,2-Difluoroethyl)-1-((5-methoxy-7-methyl-1H-indol-4-yl)methyl)piperazin-2-yl)-1H-indole-7-carboxylic acid

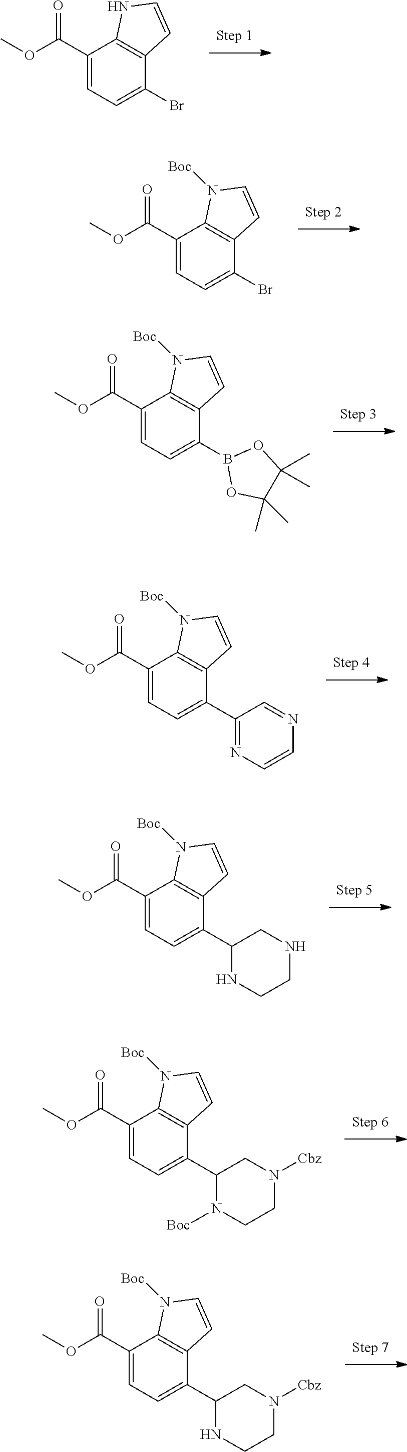

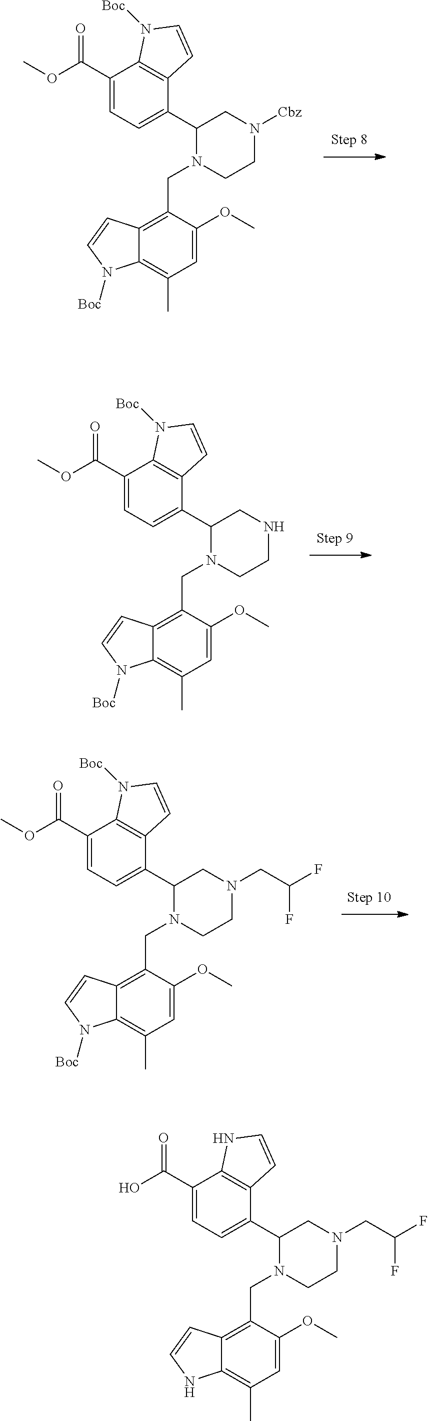

Step 1. 1-(tert-Butyl) 7-methyl 4-bromo-1H-indole-1,7-dicarboxylate was prepared with the following procedure. A 100 mL round bottom flask was charged with methyl 4-bromo-1H-indole-7-carboxylate (1.000 g, 1 eq, 3.936 mmol), THF (20 mL) and DMAP (96.17 mg, 0.2 eq, 787.2 μmol). To the stirring solution Boc₂O (1.718 g, 2.0 eq, 7.872 mmol) was added and the reaction was stirred at 20° C. for 15 h. The reaction was worked-up; concentrated under reduced pressure and the residue was purified by automated normal phase FCC to afford 1-(tert-butyl) 7-methyl 4-bromo-1H-indole-1,7-dicarboxylate (1.458 g, 105%). LCMS (General3 LCMS-4 AB) RT: 2.39 min; area % (254 nm): 95.6%; m/z=298.4 [M-tert-Butyl]⁺.

Step 2. 1-(tert-Butyl) 7-methyl 4-(4,4,5,5-tetramethyl-1,3,2-dioxaborolan-2-yl)-1H-indole-1,7-dicarboxylate was prepared with the following procedure. To a 100 mL round bottom flask containing 1-(tert-butyl) 7-methyl 4-bromo-1H-indole-1,7-dicarboxylate (1.50 g, 1.0 eq, 4.11 mmol) was dissolved in 1,4-dioxane (50 mL) and degassed with N₂, potassium acetate (1.21 g, 3.0 eq, 12.4 mmol) and bis(pinacolato)diboron (1.15 g, 1.1 eq, 4.53 mmol) were added followed by PdCl₂(dppf)-CH₂Cl₂ adduct (168 mg, 0.05 eq, 206 μmol). The mixture was heated to 80° C. and left to stir at this temperature for 15 h. The volatiles were evacuated under reduced pressure and the residue was purified by automated FCC to afford 1-(tert-butyl) 7-methyl 4-(4,4,5,5-tetramethyl-1,3,2-dioxaborolan-2-yl)-1H-indole-1,7-dicarboxylate (1.651 g, 100%). ¹H NMR (400 MHz, CDCl₃) δ 7.72 (d, J=7.4 Hz, 1H), 7.58-7.48 (m, 2H), 7.10 (d, J=3.7 Hz, 1H), 3.87 (d, J=3.0 Hz, 4H), 1.36 (s, 12H), 1.22 (s, 9H).

Step 3. 1-(tert-Butyl) 7-methyl 4-(pyrazin-2-yl)-1H-indole-1,7-dicarboxylate was prepared with the following procedure. To a 100 mL round bottom flask containing 1-(tert-butyl) 7-methyl 4-(4,4,5,5-tetramethyl-1,3,2-dioxaborolan-2-yl)-1H-indole-1,7-dicarboxylate (1.26 g, 1.0 eq, 3.15 mmol) 1,4-dioxane (50 mL) was added. The mixture was degassed for 15 minutes before adding 2-chloropyrazine (540 mg, 1.5 eq, 4.72 mmol), Pd(PPh₃)₄ (363 mg, 0.1 eq, 315 μmol) and potassium carbonate (478 mg, 1.1 eq, 3.46 mmol). The resulting mixture was allowed to stir at 100° C. for 15 h. The volatiles were evacuated under reduced pressure and the residue was purified by automated FCC to afford 1-(tert-butyl) 7-methyl 4-(pyrazin-2-yl)-1H-indole-1,7-dicarboxylate (1.27 g, 114%). LCMS (General3 LCMS-4 AB) RT: 2.39 min; area % (254 nm): 58%; m/z=354.4 [M+H]⁺

Step 4. 1-(tert-Butyl) 7-methyl 4-(piperazin-2-yl)-1H-indole-1,7-dicarboxylate was prepared with the following procedure. A 100 mL round bottom flask was charged with 1-(tert-butyl)₇-methyl 4-(pyrazin-2-yl)-1H-indole-1,7-dicarboxylate (1.16 g, 1 eq, 3.28 mmol) and AcOH (30 mL) was added. The mixture was flushed with N₂ and PdOAc₂ (147 mg, 0.2 Eq, 657 μmol) was added. The resulting mixture was allowed to stir under 1 bar of H₂ at 20° C. for 15 h. The reaction was worked-up; filtered over Celite® and the filtrate was concentrated under reduced pressure. The residue was taken up in H₂O and the pH was adjusted to 11 with the use of 30% aq. NaOH. The aqueous layer was extracted using DCM (3×40 mL). The combined organic layer was dried over Na₂SO₄, filtered, and concentrated under reduced pressure to obtain 1-(tert-butyl) 7-methyl 4-(piperazin-2-yl)-1H-indole-1,7-dicarboxylate (1.02 g, 86%). LCMS (General3 LCMS-4 AB) RT: 1.50 min; area % (254 nm): 74.2%; m/z=360.4 [M+H]⁺

Step 5. 1-(tert-Butyl) 7-methyl 4-(4-((benzyloxy)carbonyl)-1-(tert-butoxycarbonyl)piperazin-2-yl)-1H-indole-1,7-dicarboxylate was prepared with the following procedure. To a 250 mL round bottom flask containing 1-(tert-butyl) 7-methyl 4-(piperazin-2-yl)-1H-indole-1,7-dicarboxylate (1.02 g, 1.0 eq, 2.84 mmol), CH₃CN (45 mL), H₂O (15 mL) and DIPEA (983 μL, 2 eq, 5.64 mmol) were added. The mixture was cooled to 0° C. and Cbz-Cl (403 μL, 1 eq, 2.82 mmol) was added. The mixture was allowed to warm up to 20° C. and stirred at this temperature for 2 h. Boc₂O (681 mg, 717 μL, 1.1 eq, 3.12 mmol) and DIPEA (989 μL, 2 eq, 5.68 mmol) were added and the mixture was allowed to stir at 20° C. for 72 h. The volatiles were evacuated under reduced pressure and the residue was purified by automated FCC to afford 1-(tert-butyl) 7-methyl 4-(4-((benzyloxy)carbonyl)-1-(tert-butoxycarbonyl)piperazin-2-yl)-1H-indole-1,7-dicarboxylate (0.896 g, 53%). LCMS (General3 LCMS-4 AB) RT: 2.43 min; area % (combined peaks) (254 nm): 84.5%; m/z=611.2 [M+H₂O]⁺.

Step 6. 1-(tert-Butyl) 7-methyl 4-(4-((benzyloxy)carbonyl)piperazin-2-yl)-1H-indole-1,7-dicarboxylate hydrochloride was prepared with the following procedure. To a 250 mL round bottom flask containing 1-(tert-butyl) 7-methyl 4-(4-((benzyloxy)carbonyl)-1-(tert-butoxycarbonyl)piperazin-2-yl)-1H-indole-1,7-dicarboxylate (896 mg, 1.0 eq, 1.51 mmol), DCM (10 mL) and 4N HCl in dioxane (3.77 mL, 10 eq, 15.1 mmol) were added. The resulting mixture was allowed to stir at 20° C. for 4 hrs. The volatiles were evacuated under reduced pressure to afford crude 1-(tert-butyl) 7-methyl 4-(4-((benzyloxy)carbonyl)piperazin-2-yl)-1H-indole-1,7-dicarboxylate hydrochloride (710 mg, 88%).

Step 7. 1-(tert-Butyl) 7-methyl 4-(4-((benzyloxy)carbonyl)-1-((1-(tert-butoxycarbonyl)-5-methoxy-7-methyl-1H-indol-4-yl)methyl)piperazin-2-yl)-1H-indole-1,7-dicarboxylate was prepared with the following procedure. To a 250 mL round bottom flask containing crude 1-(tert-butyl) 7-methyl 4-(4-((benzyloxy)carbonyl)piperazin-2-yl)-1H-indole-1,7-dicarboxylate hydrochloride (710 mg, 1.0 eq, 1.34 mmol), DCE (20 mL) and Et₃N (747 μL, 4.0 eq, 5.36 mmol) were added. The mixture was allowed to stir at 20° C. for 30 minutes before adding AcOH (230 μL, 3 eq, 4.02 mmol) and tert-butyl 4-formyl-5-methoxy-7-methyl-1H-indole-1-carboxylate (775 mg, 2.0 eq, 2.68 mmol). The mixture was allowed to stir at 20° C. for 15 h before adding sodium triacetoxyborohydride (1.14 g, 4.0 eq, 5.36 mmol). The resulting mixture was allowed to stir at 20° C. for 15 h. The reaction was worked-up; quenched with saturated NaHCO₃ and the aqueous layer was extracted with EtOAc. The combined organic layer was dried over Na₂SO₄, filtered, and concentrated under reduced pressure. The crude was purified by automated FCC to afford 1-(tert-butyl) 7-methyl 4-(4-((benzyloxy)carbonyl)-1-((1-(tert-butoxycarbonyl)-5-methoxy-7-methyl-1H-indol-4-yl)methyl)piperazin-2-yl)-1H-indole-1,7-dicarboxylate (160 mg, 16%). LCMS (General 2 Basic) RT: 2.29 min; area % (254 nm): 84%; m/z=667.4 [M−Boc+H]⁺

Step 8. 1-(tert-butyl) 7-methyl 4-(1-((1-(tert-butoxycarbonyl)-5-methoxy-7-methyl-1H-indol-4-yl)methyl)piperazin-2-yl)-1H-indole-1,7-dicarboxylate was prepared with the following procedure. To a 100 mL round bottom flask containing 1-(tert-butyl) 7-methyl 4-(4-((benzyloxy)carbonyl)-1-((1-(tert-butoxycarbonyl)-5-methoxy-7-methyl-1H-indol-4-yl)methyl)piperazin-2-yl)-1H-indole-1,7-dicarboxylate (160 mg, 1.0 eq, 209 μmol), TFE (5 mL) was added and the mixture was flushed with N₂. 10% Pd/C (66.6 mg, 0.30 eq, 62.6 μmol) was added and the mixture was allowed to stir under H₂ atmosphere of 1 bar for 15 h. The reaction was worked-up; filtered over Celite® and the filtrate was concentrated under reduced pressure. The crude was purified by automated FCC to afford 1-(tert-butyl) 7-methyl 4-(1-((1-(tert-butoxycarbonyl)-5-methoxy-7-methyl-1H-indol-4-yl)methyl)piperazin-2-yl)-1H-indole-1,7-dicarboxylate (44.5 mg, 34%). LCMS (General3 LCMS-4 AB) RT: 2.57 min; area % (254 nm): 77%; m/z=633.2 [M+H]⁺

Step 9. 1-(tert-butyl) 7-methyl 4-(1-((1-(tert-butoxycarbonyl)-5-methoxy-7-methyl-1H-indol-4-yl)methyl)-4-(2,2-difluoroethyl)piperazin-2-yl)-1H-indole-1,7-dicarboxylate was prepared with the following procedure. To a 100 mL round bottom flask containing 1-(tert-butyl) 7-methyl 4-(1-((1-(tert-butoxycarbonyl)-5-methoxy-7-methyl-1H-indol-4-yl)methyl)piperazin-2-yl)-1H-indole-1,7-dicarboxylate (44.5 mg, 1.0 eq, 70.3 μmol) THF (1 mL) was added. To the solution Et₃N (41.2 μL, 4.2 eq, 295 μmol) and 2,2-difluoroethyl trifluoromethanesulphonate (45.2 mg, 3 eq, 211 μmol) were added. The resulting mixture was allowed to stir at 20° C. for 15 h. The reaction was worked-up; partitioned between H₂O and DCM. The aqueous layer was washed with DCM and the combined organic layer was dried over Na₂SO₄, filtered and concentrated in vacuo to afford 1-(tert-butyl) 7-methyl 4-(1-((1-(tert-butoxycarbonyl)-5-methoxy-7-methyl-1H-indol-4-yl)methyl)piperazin-2-yl)-1H-indole-1,7-dicarboxylate (52.4 mg, 107%). LCMS (General3 LCMS-4 AB) RT: 2.38 min; area % (254 nm): 56.2%; m/z=595.3 [M−H−Boc]⁻

Step 10. 4-(4-(2,2-difluoroethyl)-1-((5-methoxy-7-methyl-1H-indol-4-yl)methyl)piperazin-2-yl)-1H-indole-7-carboxylic acid was prepared following General Experimental Procedure 8. Obtained 4-(4-(2,2-difluoroethyl)-1-((5-methoxy-7-methyl-1H-indol-4-yl)methyl)piperazin-2-yl)-1H-indole-7-carboxylic acid (16 mg, 44%) as a white solid. LCMS (31697 LCMS-5 C3) RT: 1.92 min; area % (215 nm): 88.7%; m/z=483.2 [M+H]⁺. ¹H NMR (400 MHz, CD₃OD) δ 7.90 (d, J=7.7 Hz, 1H), 7.46 (d, J=7.6 Hz, 1H), 7.38 (d, J=3.2 Hz, 1H), 7.15 (d, J=3.1 Hz, 1H), 6.80 (s, 1H), 6.40 (d, J=3.1 Hz, 1H), 5.97 (tt, J=55.9, 4.3 Hz, 1H), 4.06 (s, 1H), 3.89 (d, J=11.9 Hz, 1H), 3.75 (s, 3H), 3.41 (d, J=12.0 Hz, 1H), 3.01-2.87 (m, 3H), 2.87-2.63 (m, 4H), 2.53-2.37 (m, 5H).

### Example 74. Enantiomer 1 of 2-amino-4-(4-((5-methoxy-7-methyl-1H-indol-4-yl)methyl)-1-methyl-1,4-diazepan-5-yl)benzoic acid

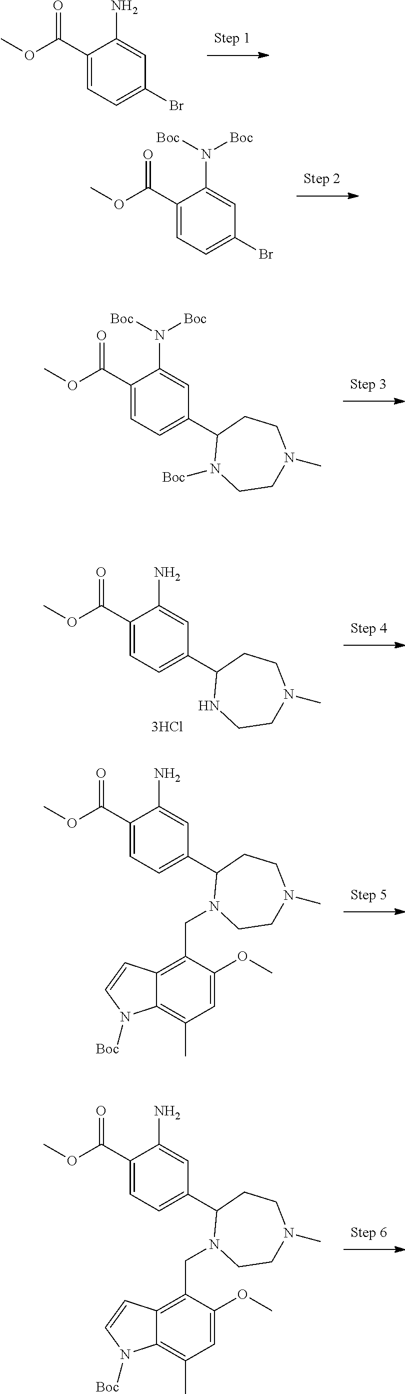

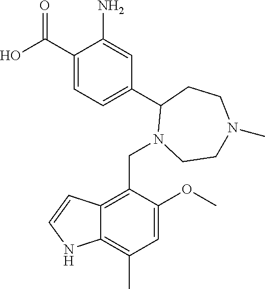 [SMILES: COc1cc(C)c2[nH]ccc2c1CN1CCN(C)CCC1c1ccc(C(=O)O)c(N)c1]

Step 1. Methyl 2-(bis(tert-butoxycarbonyl)amino)-4-bromobenzoate was prepared with the following procedure. A 100 mL round bottom flask was charged with methyl 2-amino-4-bromobenzoate (25.4 g, 1.0 eq, 110 mmol), THF (200 mL) and N,N-dimethylpyridin-4-amine (2.69 g, 0.20 eq, 22.0 mmol). To the stirring solution Boc₂O (48.1 g, 2.0 eq, 220 mmol) was added and the mixture was allowed to stir at 20° C. for 15 h. The reaction was worked up; quenched with sat. aq. NaHCO₃ (10 mL) and the organic layer was allowed to separate. The aqueous layer was extracted with DCM. The combined organic layers were dried over Na₂SO₄, filtered, and concentrated under reduced pressure to afford crude methyl 2-(bis(tert-butoxycarbonyl)amino)-4-bromobenzoate (53.2 g, 112%). ¹H NMR (400 MHz, CDCl₃) δ 7.86 (dd, J=8.4, 6.3 Hz, 1H), 7.52 (ddd, J=8.4, 7.7, 2.0 Hz, 1H), 7.34 (d, J=1.9 Hz, 1H), 3.84 (s, 3H), 1.36 (s, 18H).

Step 2. tert-Butyl 7-(3-(bis(tert-butoxycarbonyl)amino)-4-(methoxycarbonyl)phenyl)-4-methyl-1,4-diazepane-1-carboxylate was prepared with the following procedure. To a flame-dried, 500 mL 3-necked round bottom flask, under nitrogen, a solution of tert-butyl 4-methyl-1,4-diazepane-1-carboxylate (7.50 g, 1.5 eq, 35.0 mmol), TMEDA (5.58 mL, 1.6 eq, 37.2 mmol) and anhydrous Et₂O (150 mL) was cooled to −78° C. and 1.3 M sec-butyllithium in cyclohexane/hexane (28.6 mL, 1.6 eq, 37.2 mmol) was added slowly via syringe. The reaction mixture was allowed to stir at this temperature for 2 h before 0.7 M ZnCl₂ in THF (53.1 mL, 1.6 eq, 37.2 mmol) was added. The reaction mixture was allowed to warm to 20° C. and stirred at this temperature for 2 h. In the meantime a solution of Pd(PPh₃)₄ (1.61 g, 0.060 eq, 1.39 mmol) and 2-dicyclohexylphosphino-2,6-dimethoxy-1,1-biphenyl (1.17 g, 0.12 Eq, 2.86 mmol) was prepared in anhydrous toluene (300 mL) and allowed to stir at 20° C. for 15 minutes. The catalyst solution was added to the piperidinyl zinc reagent followed by methyl 2-(bis(tert-butoxycarbonyl)amino)-4-bromobenzoate (10.0 g, 1.0 eq, 23.2 mmol). The resulting mixture was heated to 110° C. and allowed to stir at this temperature for 15 h. The reaction was worked-up; quenched with sat. aq. NH₄Cl and the aqueous layer was extracted with DCM. The combined organic layers were dried over Na₂SO₄, filtered, and concentrated under reduced pressure. The crude was purified by automated normal phase FCC to afford tert-butyl 7-(3-(bis(tert-butoxycarbonyl)amino)-4-(methoxycarbonyl)phenyl)-4-methyl-1,4-diazepane-1-carboxylate (818 mg, 6.2%). LCMS (LCMS-4 General3 AB) RT: 2.35 min; area % (254 nm): 75.5%; m/z=464.4 [M−Boc]⁺.

Step 3. Methyl 2-amino-4-(1-methyl-1,4-diazepan-5-yl)benzoate trihydrochloride was prepared with the following procedure. To a 250 mL round bottom flask containing tert-butyl 7-(3-((tert-butoxycarbonyl)amino)-4-(methoxycarbonyl)phenyl)-4-methyl-1,4-diazepane-1-carboxylate (640 mg, 1.0 eq, 1.38 mmol), DCM (20 mL) and 4 N HCl in dioxane (3.45 mL, 10 eq, 13.8 mmol) were added. The resulting mixture was allowed to stir at 20° C. for 72 h. The volatiles were evacuated under reduced pressure to afford crude methyl 2-amino-4-(1-methyl-1,4-diazepan-5-yl)benzoate trihydrochloride (520 mg, 101%).

Step 4. tert-Butyl 4-((7-(3-amino-4-(methoxycarbonyl)phenyl)-4-methyl-1,4-diazepan-1-yl)methyl)-5-methoxy-7-methyl-1H-indole-1-carboxylate was prepared with the following procedure. To a 250 mL round bottom flask containing methyl 2-amino-4-(1-methyl-1,4-diazepan-5-yl)benzoate trihydrochloride (515 mg, 1.0 eq, 1.38 mmol), DCE (30 mL) and Et-3N (963 μL, 5.0 eq, 6.91 mmol) were added. The mixture was allowed to stir at 20° C. for 30 minutes before adding tert-butyl 4-formyl-5-methoxy-7-methyl-1H-indole-1-carboxylate (520 mg, 1.3 eq, 1.80 mmol) and acetic acid (158 μL, 2.0 eq, 2.76 mmol). The mixture was allowed to stir at 20° C. for 3 hours before adding sodium triacetoxyhydroborate (1.17 g, 4.0 eq, 5.53 mmol). The resulting suspension was allowed to stir at 20° C. for 15 h. The reaction was worked-up; quenched with NaHCO₃ and the aqueous layer was extracted with EtOAc. The combined organic layers were dried over Na₂SO₄, filtered, and concentrated under reduced pressure. The crude was purified by automated normal phase FCC to afford tert-butyl 4-((7-(3-amino-4-(methoxycarbonyl)phenyl)-4-methyl-1,4-diazepan-1-yl)methyl)-5-methoxy-7-methyl-1H-indole-1-carboxylate (630 mg, 85%). LCMS (LCMS-4 General3 AB) RT: 2.33 min; area % (254 nm): 79.1%; m/z=537.4 [M+H]⁺.

Step 5. Enantiomer 1 of tert-butyl 4-((7-(3-amino-4-(methoxycarbonyl)phenyl)-4-methyl-1,4-diazepan-1-yl)methyl)-5-methoxy-7-methyl-1H-indole-1-carboxylate was prepared with the following procedure. Chiral SFC purification (120 mL 15% B) was performed on racemic tert-butyl 4-((7-(3-amino-4-(methoxycarbonyl)phenyl)-4-methyl-1,4-diazepan-1-yl)methyl)-5-methoxy-7-methyl-1H-indole-1-carboxylate (630 mg, 1.17 mmol) to afford enantiomer 1 of tert-butyl 4-((7-(3-amino-4-(methoxycarbonyl)phenyl)-4-methyl-1,4-diazepan-1-yl)methyl)-5-methoxy-7-methyl-1H-indole-1-carboxylate (124 mg, 19.7%). First eluting enantiomer (31697V UPC² 7m Cell MA1) RT: 4.69 min; area % (215 nm): 95.6% ee, m/z=537.3 [M+H]⁺.

Step 6. Enantiomer 1 of 2-amino-4-(4-((5-methoxy-7-methyl-1H-indol-4-yl)methyl)-1-methyl-1,4-diazepan-5-yl)benzoic acid was prepared following General Experimental Procedure 8. Enantiomer 1 of tert-butyl 4-((7-(3-amino-4-(methoxycarbonyl)phenyl)-4-methyl-1,4-diazepan-1-yl)methyl)-5-methoxy-7-methyl-1H-indole-1-carboxylate (124 mg) gave enantiomer 1 of 2-amino-4-(4-((5-methoxy-7-methyl-1H-indol-4-yl)methyl)-1-methyl-1,4-diazepan-5-yl)benzoic acid (50 mg, 51%) as a white solid. LCMS (31697 LCMS-5 C3) RT: 1.44 min; area % (215 nm): 96.8%; m/z=423.2 [M+H]⁺. ¹H NMR (400 MHz, CD₃OD) δ 7.87 (d, J=8.1 Hz, 1H), 7.21 (d, J=3.1 Hz, 1H), 6.86 (d, J=1.7 Hz, 1H), 6.76 (dd, J=8.0, 1.8 Hz, 1H), 6.71 (s, 1H), 6.28 (d, J=3.1 Hz, 1H), 4.03-3.91 (m, 3H), 3.78 (s, 3H), 3.12-3.01 (m, 3H), 2.95 (dd, J=13.5, 7.4 Hz, 1H), 2.66 (s, 3H), 2.47 (d, J=0.8 Hz, 3H), 2.20 (s, 2H).

### Example 74. Enantiomer 2 of 2-amino-4-(4-((5-methoxy-7-methyl-1H-indol-4-yl)methyl)-1-methyl-1,4-diazepan-5-yl)benzoic acid

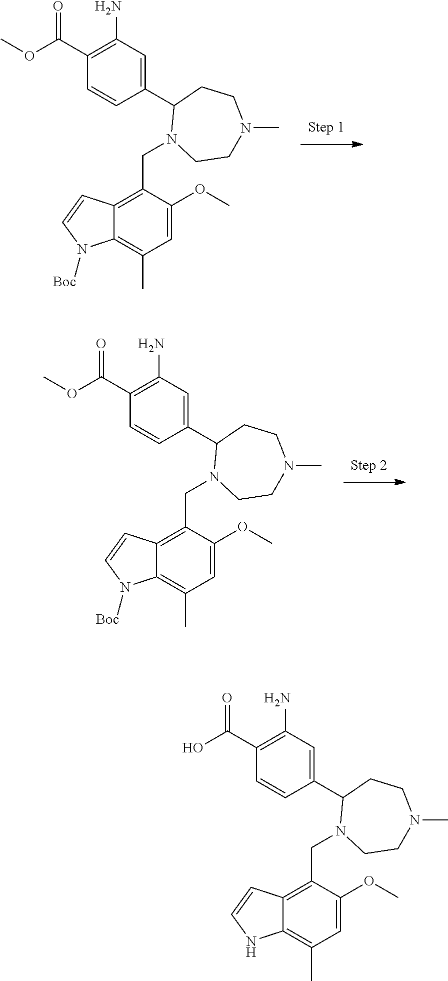

Step 1. Enantiomer 2 of tert-butyl 4-((7-(3-amino-4-(methoxycarbonyl)phenyl)-4-methyl-1,4-diazepan-1-yl)methyl)-5-methoxy-7-methyl-1H-indole-1-carboxylate was prepared with the following procedure. Chiral SFC purification (120 mL 15% B) was performed on racemic tert-butyl 4-((7-(3-amino-4-(methoxycarbonyl)phenyl)-4-methyl-1,4-diazepan-1-yl)methyl)-5-methoxy-7-methyl-1H-indole-1-carboxylate (630 mg, 1.17 mmol) to afford enantiomer 1 of tert-butyl 4-((7-(3-amino-4-(methoxycarbonyl)phenyl)-4-methyl-1,4-diazepan-1-yl)methyl)-5-methoxy-7-methyl-1H-indole-1-carboxylate (72 mg, 11%). Second eluting enantiomer (31697V UPC² 7m Cell MA1) RT: 4.84 min; area % (215 nm): 98.7% ee, m/z=537.3 [M+H]⁺.

Step 2. Enantiomer 2 of 2-amino-4-(4-((5-methoxy-7-methyl-1H-indol-4-yl)methyl)-1-methyl-1,4-diazepan-5-yl)benzoic acid was prepared following General Experimental Procedure 1. Enantiomer 2 of tert-butyl 4-((7-(3-amino-4-(methoxycarbonyl)phenyl)-4-methyl-1,4-diazepan-1-yl)methyl)-5-methoxy-7-methyl-1H-indole-1-carboxylate (124 mg) gave enantiomer 2 of 2-amino-4-(4-((5-methoxy-7-methyl-1H-indol-4-yl)methyl)-1-methyl-1,4-diazepan-5-yl)benzoic acid (38 mg, 68%) as a white solid. LCMS (31697 LCMS-5 C3) RT: 1.46 min; area % (215 nm): 98.8%; m/z=423.2 [M+H]⁺. ¹H NMR (400 MHz, CD₃OD) δ 7.87 (d, J=8.1 Hz, 1H), 7.21 (d, J=3.1 Hz, 1H), 6.86 (d, J=1.7 Hz, 1H), 6.76 (dd, J=8.0, 1.8 Hz, 1H), 6.71 (s, 1H), 6.28 (d, J=3.1 Hz, 1H), 4.03-3.91 (m, 3H), 3.78 (s, 3H), 3.12-3.01 (m, 3H), 2.95 (dd, J=13.5, 7.4 Hz, 1H), 2.66 (s, 3H), 2.47 (d, J=0.8 Hz, 3H), 2.20 (s, 2H).

### Example 75. Diastereomer 1 of 4-((8aR)-2-((5-methoxy-7-methyl-1H-indol-4-yl)methyl)octahydropyrrolo[1,2-a]pyrazin-3-yl)benzoic acid

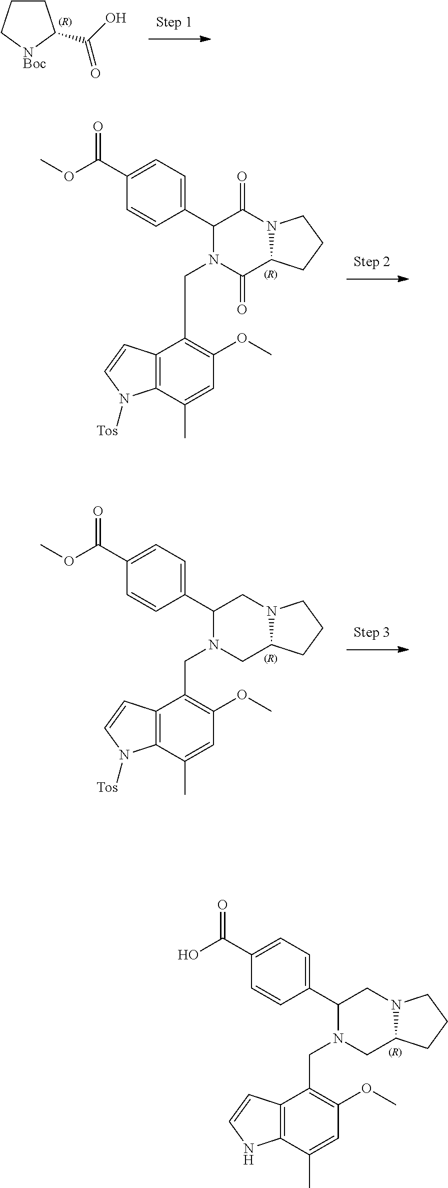

Step 1. Methyl 4-((8aR)-2-((5-methoxy-7-methyl-1-tosyl-1H-indol-4-yl)methyl)-1,4-dioxooctahydropyrrolo[1,2-a]pyrazin-3-yl)benzoate was prepared following General Experimental Procedure 9. Methyl 4-formylbenzoate (477 mg, 2.90 mmol), (5-methoxy-7-methyl-1-tosyl-1H-indol-4-yl)methanamine (1.00 g, 2.90 mmol), (tert-butoxycarbonyl)-d-proline (625 mg, 2.90 mmol), and 2-(2,2-dimethoxyethyl)-N-methylidynebenzenaminium (510 L, 2.90 mmol) gave diastereomer 1 of methyl 4-((8aR)-2-((5-methoxy-7-methyl-1-tosyl-1H-indol-4-yl)methyl)-1,4-dioxooctahydropyrrolo[1,2-a]pyrazin-3-yl)benzoate (180 mg, 10%) as a yellow oil. LCMS (General 3 Basic) RT: 1.82 min; area % (254 nm): 76%; m/z=616.3 [M+H]⁺.

Step 2. Diastereomer 1 of methyl 4-((8aR)-2-((5-methoxy-7-methyl-1-tosyl-1H-indol-4-yl)methyl)octahydropyrrolo[1,2-a]pyrazin-3-yl)benzoate was prepared following General Experimental Procedure 10. Diastereomer 1 of methyl 4-((8aR)-2-((5-methoxy-7-methyl-1-tosyl-1H-indol-4-yl)methyl)-1,4-dioxooctahydropyrrolo[1,2-a]pyrazin-3-yl)benzoate (180 mg, 292 μmol), gave diastereomer 1 of methyl 4-((8aR)-2-((5-methoxy-7-methyl-1-tosyl-1H-indol-4-yl)methyl)octahydropyrrolo[1,2-a]pyrazin-3-yl)benzoate (45 mg, 26%). LCMS (General 3 Basic) RT: 2.22 min; area % (254 nm): 92%; m/z=588.3 [M+H]⁺.

Step 3. Diastereomer 1 of 4-((8aR)-2-((5-methoxy-7-methyl-1H-indol-4-yl)methyl)octahydropyrrolo[1,2-a]pyrazin-3-yl)benzoic acid was prepared following General Experimental Procedure 11. Diastereomer 1 of methyl 4-((8aR)-2-((5-methoxy-7-methyl-1-tosyl-1H-indol-4-yl)methyl)octahydropyrrolo[1,2-a]pyrazin-3-yl)benzoate (45 mg, 16 μmol) gave diastereomer 1 of 4-((8aR)-2-((5-methoxy-7-methyl-1H-indol-4-yl)methyl)octahydropyrrolo[1,2-a]pyrazin-3-yl)benzoic acid (1.61 mg, 4%) as a white solid. LCMS (31697 LCMS-5 C3) RT: 1.587 min; area % (215 nm): 98.8%; m/z=420.2 [M+H]⁺. ¹H-NMR (400 MHz, CD₃OD) δ 8.06 (d, J=8.1 Hz, 2H), 7.64 (d, J=7.7 Hz, 2H), 7.22 (d, J=3.1 Hz, 1H), 6.70 (s, 1H), 6.39 (d, J=3.1 Hz, 1H), 4.58 (s, 2H), 3.98-3.90 (m, 1H), 3.76 (s, 3H), 3.63-3.53 (m, 1H), 3.25 (d, J=12.4 Hz, 2H), 3.18-3.11 (m, 1H), 2.67 (d, J=9.5 Hz, 1H), 2.57 (s, 1H), 2.47 (d, J=0.8 Hz, 3H), 1.87 (d, J=8.0 Hz, 3H), 1.50-1.41 (m, 1H), 1.29 (s, 1H).

### Example 76. Diastereomer 2 of 4-((8aR)-2-((5-methoxy-7-methyl-1H-indol-4-yl)methyl)octahydropyrrolo[1,2-a]pyrazin-3-yl)benzoic acid

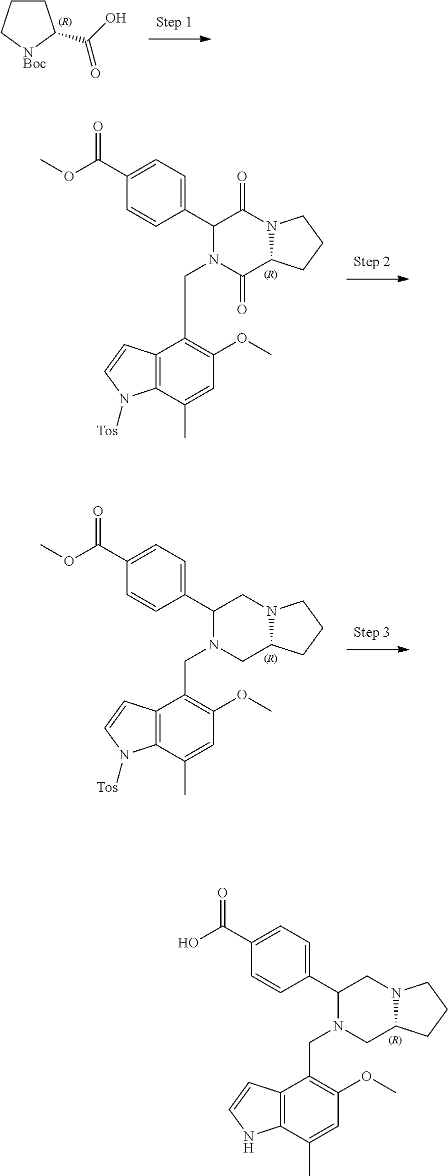

Step 1. Methyl 4-((8aR)-2-((5-methoxy-7-methyl-1-tosyl-1H-indol-4-yl)methyl)-1,4-dioxooctahydropyrrolo[1,2-a]pyrazin-3-yl)benzoate was prepared following General Experimental Procedure 9. Methyl 4-formylbenzoate (477 mg, 2.90 mmol), (5-methoxy-7-methyl-1-tosyl-1H-indol-4-yl)methanamine (1.00 g, 2.90 mmol), (tert-butoxycarbonyl)-d-proline (625 mg, 2.90 mmol), and 2-(2,2-dimethoxyethyl)-N-methylidynebenzenaminium (510 μL, 2.90 mmol) gave diastereomer 2 of methyl 4-((8aR)-2-((5-methoxy-7-methyl-1-tosyl-1H-indol-4-yl)methyl)-1,4-dioxooctahydropyrrolo[1,2-a]pyrazin-3-yl)benzoate (150 mg, 8%) as a yellow oil. LCMS (General 3 Basic) RT: 1.88 min; area % (254 nm): 76%; m/z=616.2 [M+H]⁺.

Step 2. Diastereomer 2 of methyl 4-((8aR)-2-((5-methoxy-7-methyl-1-tosyl-1H-indol-4-yl)methyl)octahydropyrrolo[1,2-a]pyrazin-3-yl)benzoate was prepared following General Experimental Procedure 10. Diastereomer 2 of methyl 4-((8aR)-2-((5-methoxy-7-methyl-1-tosyl-1H-indol-4-yl)methyl)-1,4-dioxooctahydropyrrolo[1,2-a]pyrazin-3-yl)benzoate (180 mg, 292 μmol) gave diastereomer 2 of methyl 4-((8aR)-2-((5-methoxy-7-methyl-1-tosyl-1H-indol-4-yl)methyl)octahydropyrrolo[1,2-a]pyrazin-3-yl)benzoate (30 mg, 17%). LCMS (General 3 Basic) RT: 2.23 min; area % (254 nm): 55%; m/z=588.3 [M+H]⁺.

Step 3. Diastereomer 2 of 4-((8aR)-2-((5-methoxy-7-methyl-1H-indol-4-yl)methyl)octahydropyrrolo[1,2-a]pyrazin-3-yl)benzoic acid was prepared following General Experimental Procedure 11. Diastereomer 2 of methyl 4-((8aR)-2-((5-methoxy-7-methyl-1-tosyl-1H-indol-4-yl)methyl)octahydropyrrolo[1,2-a]pyrazin-3-yl)benzoate (30 mg, 11 μmol) gave diastereomer 2 of 4-((8aR)-2-((5-methoxy-7-methyl-1H-indol-4-yl)methyl)octahydropyrrolo[1,2-a]pyrazin-3-yl)benzoic acid (1.03 mg, 5%) as a white solid. LCMS (31697 LCMS-5 C3) RT: 1.588 min; area % (215 nm): 91.3%; m/z=420.2 [M+H]⁺. ¹H-NMR (400 MHz, CD₃OD) δ 8.08 (d, J=8.1 Hz, 2H), 7.65 (d, J=7.8 Hz, 2H), 7.23 (d, J=3.1 Hz, 1H), 6.71 (s, 1H), 6.39 (d, J=3.2 Hz, 1H), 4.58 (s, 1H), 4.02-3.91 (m, 2H), 3.76 (s, 3H), 3.69-3.54 (m, 2H), 2.75 (s, 2H), 2.66 (s, 2H), 2.47 (d, J=0.8 Hz, 3H), 1.89 (d, J=8.4 Hz, 4H), 1.45 (d, J=19.2 Hz, 3H), 1.27 (d, J=18.2 Hz, 3H).

### Example 77. 4-((9aR)-3-((5-methoxy-7-methyl-1H-indol-4-yl)methyl)octahydro-1H-pyrrolo[1,2-d][1,4]diazepin-4-yl)benzoic acid

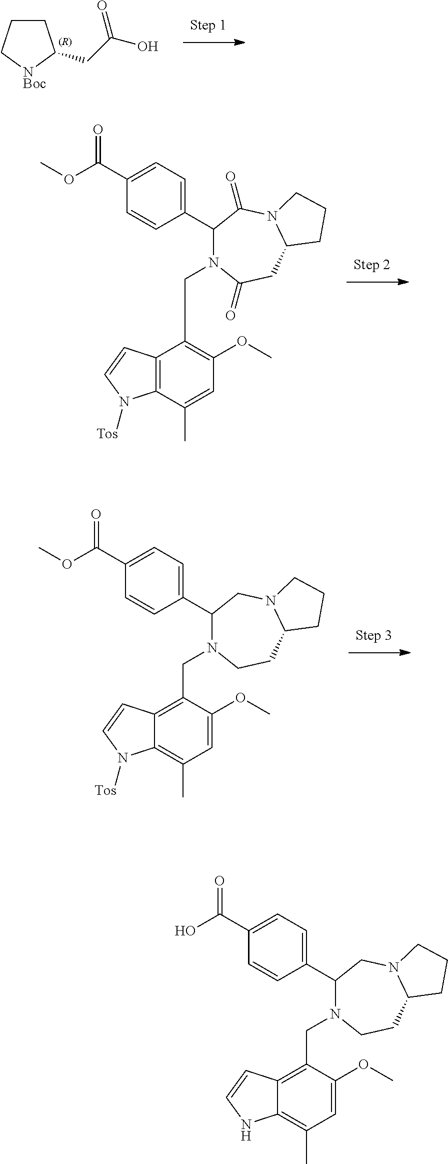

Step 1. Methyl 4-((9aR)-3-((5-methoxy-7-methyl-1-tosyl-1H-indol-4-yl)methyl)-2,5-dioxooctahydro-1H-pyrrolo[1,2-d][1,4]diazepin-4-yl)benzoate was prepared following General Experimental Procedure 9, with the exception that the resulting residue was dissolved in toluene, Et₃N (50 eq.) added and the mixture heated at 100° C. for 16 h. Methyl 4-formylbenzoate (238 mg, 1.45 mmol), (5-methoxy-7-methyl-1-tosyl-1H-indol-4-yl)methanamine (500 mg, 1.45 mmol), 2-(2,2-dimehtoxyethyl)-N-methylynebenzaminium (0.25 mL, 1.45 mmol), and (R)-2-(1-(tert-butoxycarbonyl)pyrrolidin-2-yl) acetic acid (333 mg, 1.45 mmol) gave methyl 4-((9aR)-3-((5-methoxy-7-methyl-1-tosyl-1H-indol-4-yl)methyl)-2,5-dioxooctahydro-1H-pyrrolo[1,2-d][1,4]diazepin-4-yl)benzoate (99 mg, 11%). LCMS (General 3 Basic) RT: 1.82 min; area % (254 nm): 64%; m/z=630.4 [M+H]⁺.

Step 2. Methyl 4-((9aR)-3-((5-methoxy-7-methyl-1-tosyl-1H-indol-4-yl)methyl)octahydro-1H-pyrrolo[1,2-d][1,4]diazepin-4-yl)benzoate was prepared following General Experimental Procedure 10. 4-((9aR)-3-((5-Methoxy-7-methyl-1-tosyl-1H-indol-4-yl)methyl)-2,5-dioxooctahydro-1H-pyrrolo[1,2-d][1,4]diazepin-4-yl)benzoate (99 mg, 0.16 mmol) gave methyl 4-((9aR)-3-((5-methoxy-7-methyl-1-tosyl-1H-indol-4-yl)methyl)octahydro-1H-pyrrolo[1,2-d][1,4]diazepin-4-yl)benzoate (95 mg, 100%). LCMS (General 3 Acidic) RT: 1.14 min; area % 96%; m/z=602.5 [M+H]⁺.

Step 3. 4-((9aR)-3-((5-Methoxy-7-methyl-1H-indol-4-yl)methyl)octahydro-1H-pyrrolo[1,2-d][1,4]diazepin-4-yl)benzoic acid was prepared with the following procedure. To a solution of methyl 4-((9aR)-3-((5-methoxy-7-methyl-1-tosyl-1H-indol-4-yl)methyl)octahydro-1H-pyrrolo[1,2-d][1,4]diazepin-4-yl)benzoate (50 mg, 1.0 eq, 83 μmol) in THF (2 mL) and MeOH (2 mL) was added magnesium (20 mg, 10 eq, 0.83 mmol) and ammonium chloride (8.3 mg, 2 eq, 0.17 mmol). The reaction mixture was stirred at 20° C. for 20 h. To the reaction mixture was added sat. aq. NH₄Cl (5 mL) and then extracted with EtOAc (2×10 mL). The organic layers were combined, dried over Na₂SO₄, filtered and then concentrated under reduced pressure. To the residue was added a mixture of MeOH:H₂O (2:1 ratio, 5 mL), lithium hydroxide monohydrate (7.0 mg, 2.0 eq, 0.17 mmol) and then THF (2 mL). The mixture was stirred at 20° C. for 20 hours, and then more lithium hydroxide monohydrate (7.0 mg, 2.0 eq, 0.17 mmol) was added. After stirring for 20 hours, acetic acid was added to the reaction mixture to neutralize to pH 7. The volatiles were removed under reduced pressure and the residue was purified by automated reverse phase FCC for purification to afford 4-((9aR)-3-((5-methoxy-7-methyl-1H-indol-4-yl)methyl)octahydro-1H-pyrrolo[1,2-d][1,4]diazepin-4-yl)benzoic acid (3.17 mg, yield 9%) as a white solid. LCMS (31697 LCMS-5 C3) RT: 1.577 min; area % (215 nm): 95%; m/z=434.3 [M+H]⁺. ¹H-NMR (400 MHz, CD₃OD) δ 8.07 (d, J=7.8 Hz, 2H), 7.60 (d, J=7.6 Hz, 2H), 7.23 (d, J=3.1 Hz, 1H), 6.70 (s, 1H), 6.37 (d, J=3.1 Hz, 1H), 4.26 (s, 1H), 3.99-3.80 (m, 2H), 3.74 (m, 4H), 3.35 (d, J=4.4 Hz, 1H), 3.18 (d, J=13.2 Hz, 2H), 3.09 (h, J=6.7 Hz, 2H), 2.92 (s, 1H), 2.46 (d, J=0.8 Hz, 3H), 2.21-2.09 (m, 1H), 2.05-1.76 (m, 4H), 1.72-1.55 (m, 1H).

### Example 78. 4-(2-((5-Methoxy-7-methyl-1H-indol-4-yl)methyl)octahydropyrrolo[1,2-a]pyrazin-1-yl)benzoic acid

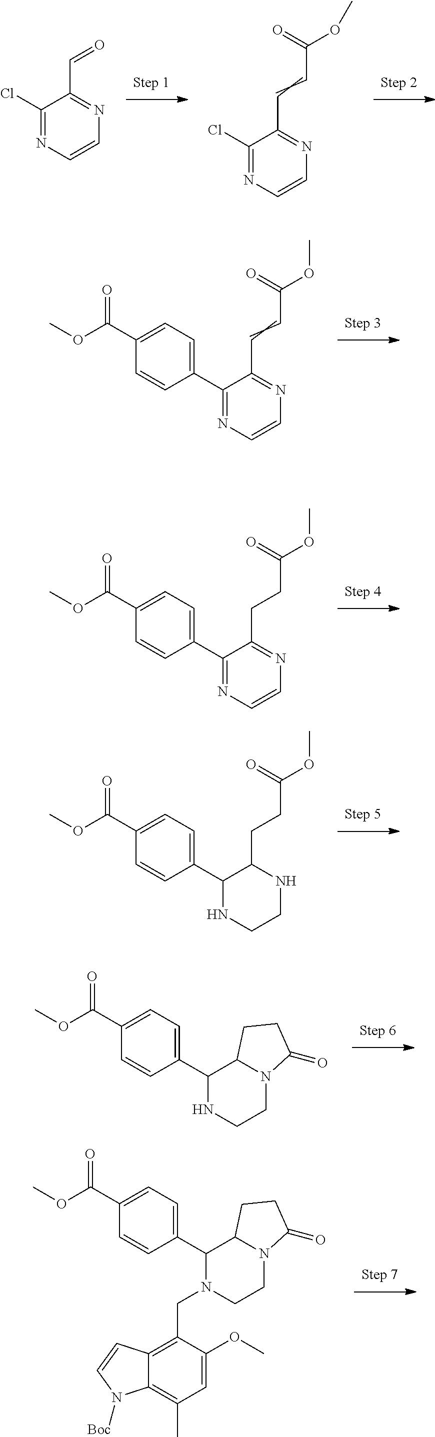

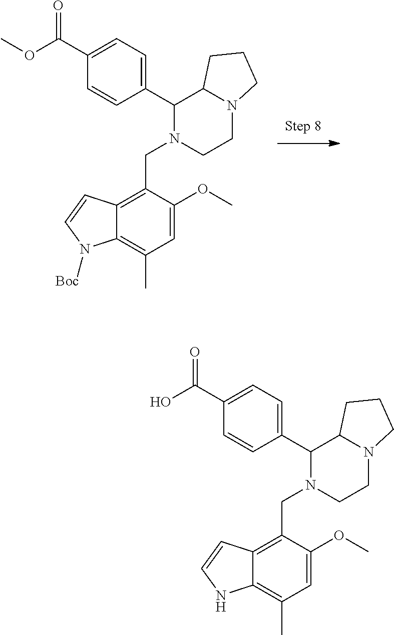 [SMILES: COC(=O)c1ccc(C2C3CCCN3CCN2Cc2c(OC)cc(C)c3c2ccn3C(=O)OC(C)(C)C)cc1 ; COc1cc(C)c2[nH]ccc2c1CN1CCN2CCCC2C1c1ccc(C(=O)O)cc1]

Step 1. Methyl 3-(3-chloropyrazin-2-yl) acrylate was prepared according to the following procedure. To 3-chloropyrazine-2-carbaldehyde (500 mg, 1.0 eq, 3.51 mmol) in toluene (230 mL) was added 60 wt. % sodium hydride dispersion in mineral oil (0.21 g, 1.5 eq. 5.26 mmol) and diethylphosphonoacetic acid ethylester (697 μL, 1.0 eq. 3.51 mmol). The mixture was stirred in the dark at 20° C. for 3 days. The mixture was quenched with H₂O (200 mL) and the aqueous layer was washed with EtOAc (total 200 mL). The combined organic layers were washed with brine (3×50 mL), dried over Na₂SO₄, filtered, and concentrated under reduced pressure. The residue was purified by automated normal phase FCC to afford the separate geometric isomers. Methyl (Z)-3-(3-chloropyrazin-2-yl) acrylate (239 mg, 32%) as a yellowish oil, which solidified on standing. ¹H-NMR (400 MHz, CDCl₃) δ 8.52 (dd, J=2.3, 0.5 Hz, 1H), 8.35 (d, J=2.3 Hz, 1H), 8.03 (dd, J=15.5, 0.4 Hz, 1H), 7.14 (d, J=15.5 Hz, 1H), 4.31 (q, J=7.1 Hz, 2H), 1.36 (t, J=7.1 Hz, 3H). Methyl (E)-3-(3-chloropyrazin-2-yl) acrylate (64 mg, 0.30 mmol, 8.6%). ¹H-NMR (400 MHz, CDCl₃) δ 8.46 (d, J=2.4 Hz, 1H), 8.30 (dd, J=2.4, 0.6 Hz, 1H), 7.11 (d, J=12.0 Hz, 1H), 6.32 (d, J=12.1 Hz, 1H), 4.17 (q, J=7.1 Hz, 2H), 1.20 (t, J=7.1 Hz, 3H).

Step 2. Methyl 4-(3-(3-methoxy-3-oxoprop-1-en-1-yl)pyrazin-2-yl)benzoate was prepared according to the following procedure. A mixture of the geometric isomers of methyl 3-(3-chloropyrazin-2-yl) acrylate (303 mg, 1.0 eq, 1.43 mmol), methyl 4-(4,4,5,5-tetramethyl-1,3,2-dioxaborolan-2-yl)benzoate (486 mg, 1.3 eq, 1.85 mmol), and 20 wt. % aq. Na₂CO₃ (892 μL, 3.0 eq, 4.28 mmol) in toluene (7.5 mL) and ethanol (2.3 mL) was bubbled with N₂ for 30 min. Then Pd(PPh₃)₄ (82 mg, 0.05 eq, 71 μmol) was added and the reaction mixture was heated to 71° C. (internal temperature) for 16 h. The reaction mixture was cooled to 20° C., H₂O was added and extracted with EtOAc (2×20 mL). The organic layers were combined dried over Na₂SO₄, filtered, and concentrated under reduced pressure. The residue was purified by automated normal phase FCC to afford purified product that was recrystallized from TBME to afford methyl 4-(3-(3-methoxy-3-oxoprop-1-en-1-yl)pyrazin-2-yl)benzoate (211 mg, 47%). ¹H-NMR (400 MHz, CDCl₃) δ 8.67-8.59 (m, 2H), 8.24-8.16 (m, 2H), 7.78-7.71 (m, 1H), 7.71-7.66 (m, 2H), 7.17 (d, J=15.4 Hz, 1H), 4.25 (q, J=7.1 Hz, 2H), 3.97 (s, 3H), 1.34-1.23 (m, 3H).

Step 3. Methyl 4-(3-(3-methoxy-3-oxopropyl)pyrazin-2-yl)benzoate was prepared as follows. A suspension of methyl 4-(3-(3-methoxy-3-oxoprop-1-en-1-yl)pyrazin-2-yl)benzoate (160 mg, 1.0 eq, 0.51 mmol) and 10% Pd/C (30 mg, 0.55 eq, 0.28 mmol) in IPA (6 mL) was flushed with hydrogen and stirred under a hydrogen atmosphere (balloon) at 40° C. for two days. The reaction was worked-up; filtered over Celite® and to the filtrate was added fresh 10% Pd/C (30 mg, 0.55 eq, 0.28 mmol). The flask was flushed with hydrogen and stirred under a hydrogen atmosphere (balloon) at 60° C. for 15 h. The reaction was worked-up; filtered over Celite®, washed the filter cake with IPA, and the filtrate concentrated under reduced pressure to afford crude methyl 4-(3-(3-methoxy-3-oxopropyl)pyrazin-2-yl)benzoate (161 mg). LCMS (LCMS-4 General 3 AB) RT 1.839 min; area % (254 nm): 65%; m/z=315.4 [M+H]⁺.

Step 4. Methyl 4-(3-(3-methoxy-3-oxopropyl)piperazin-2-yl)benzoate was prepared according to the following procedure. A mixture of crude methyl 4-(3-(3-methoxy-3-oxopropyl)pyrazin-2-yl)benzoate (141 mg) and 10% Pd/C (140 mg, 2.93 eq, 1.32 mmol) in trifluoroethanol (10 mL) was flushed with hydrogen and stirred under a hydrogen atmosphere (balloon) at 53° C. (internal temperature) for three days. The reaction was worked-up; filtered over Celite®, washed the filter cake with EtOH, and the filtrate concentrated under reduced pressure to afford crude methyl 4-(3-(3-methoxy-3-oxopropyl)piperazin-2-yl)benzoate (117 mg). LCMS (LCMS-4 General 3 AB) RT 1.162 min; area % (254 nm): 35%; m/z=321.4 [M+H]⁺.

Step 5. Methyl 4-(6-oxooctahydropyrrolo[1,2-a]pyrazin-1-yl)benzoate was prepared according to the following procedure. A solution of crude methyl 4-(3-(3-ethoxy-3-oxopropyl)piperazin-2-yl)benzoate (117 mg) and DIPEA (0.3 mL, 4.6 eq, 1.68 mmol) in toluene (5 mL) was heated at 110° C. for 16 h. The reaction was worked-up; concentrated under reduced pressure to afford crude methyl 4-(6-oxooctahydropyrrolo[1,2-a]pyrazin-1-yl)benzoate (100 mg, 99%). LCMS (LCMS-4 General 3 AB) RT 0.766 min & 1.113 min; combined area % (254 nm): 81%; m/z=275.4 [M+H]⁺.

Step 6. tert-Butyl 5-methoxy-4-((1-(4-(methoxycarbonyl)phenyl)hexahydropyrrolo[1,2-a]pyrazin-2 (1H)-yl)methyl)-7-methyl-1H-indole-1-carboxylate was prepared following General Experimental Procedure 4, with the exception that the residue was purified by automated reversed phase FCC. Crude methyl 4-(6-oxooctahydropyrrolo[1,2-a]pyrazin-1-yl)benzoate (70 mg, 0.26 mmol) and tert-butyl 4-formyl-5-methoxy-7-methyl-1H-indole-1-carboxylate (216 mg, 0.75 mmol) gave tert-butyl 5-methoxy-4-((1-(4-(methoxycarbonyl)phenyl)hexahydropyrrolo[1,2-a]pyrazin-2 (1H)-yl)methyl)-7-methyl-1H-indole-1-carboxylate (44 mg, 16% over 4 steps). LCMS (General 3 basic) RT: 2.08 min; area % (254 nm): 91%; m/z=548.5 [M+H]⁺.

Step 7. tert-Butyl 5-methoxy-4-((1-(4-(methoxycarbonyl)phenyl)hexahydropyrrolo[1,2-a]pyrazin-2 (1H)-yl)methyl)-7-methyl-1H-indole-1-carboxylate was prepared following General Experimental Procedure 10. tert-Butyl 5-methoxy-4-((1-(4-(methoxycarbonyl)phenyl)-6-oxohexahydropyrrolo[1,2-a]pyrazin-2 (1H)-yl)methyl)-7-methyl-1H-indole-1-carboxylate (44 mg, 73 μmol) gave tert-butyl 5-methoxy-4-((1-(4-(methoxycarbonyl)phenyl)hexahydropyrrolo[1,2-a]pyrazin-2 (1H)-yl)methyl)-7-methyl-1H-indole-1-carboxylate (38 mg, 97%). LCMS (General 3 basic) RT: 2.40 min; area % (254 nm): 82%; m/z=534.5 [M+H]⁺.

Step 8. 4-(2-((5-Methoxy-7-methyl-1H-indol-4-yl)methyl)octahydropyrrolo[1,2-a]pyrazin-1-yl)benzoic acid was prepared following General Experimental Procedure 8. tert-Butyl 5-methoxy-4-((1-(4-(methoxycarbonyl)phenyl)hexahydropyrrolo[1,2-a]pyrazin-2 (1H)-yl)methyl)-7-methyl-1H-indole-1-carboxylate (38 mg 71 μmol) gave 4-(2-((5-methoxy-7-methyl-1H-indol-4-yl)methyl)octahydropyrrolo[1,2-a]pyrazin-1-yl)benzoic acid (12 mg, 40%) as a white solid. LCMS (31697 LCMS-5 C3) RT: 1.358 min & 1.469 min; combined area % (215 nm): 95%; m/z=420.2 [M+H]⁺. ¹H-NMR (400 MHz, CD₃OD) δ 8.10-8.00 (m, 2H), 7.75-7.56 (m, 2H), 7.27-7.19 (m, 1H), 6.71 (s, 1H), 6.37 (d, J=3.1 Hz, 1H), 3.97 (dd, J=35.8, 13.5 Hz, 1H), 3.75 (s, 3H), 3.68-3.46 (m, 2H), 3.26-3.00 (m, 4H), 2.78 (t, J=12.1 Hz, 1H), 2.68-2.52 (m, 1H), 2.50-2.24 (m, 4H), 1.96-1.66 (m, 2H), 1.59-1.26 (m, 2H).

### Example 79. 4-(2-((5-Methoxy-7-methyl-1H-indol-4-yl)methyl)-6-oxooctahydropyrrolo[1,2-a]pyrazin-1-yl)benzoic acid

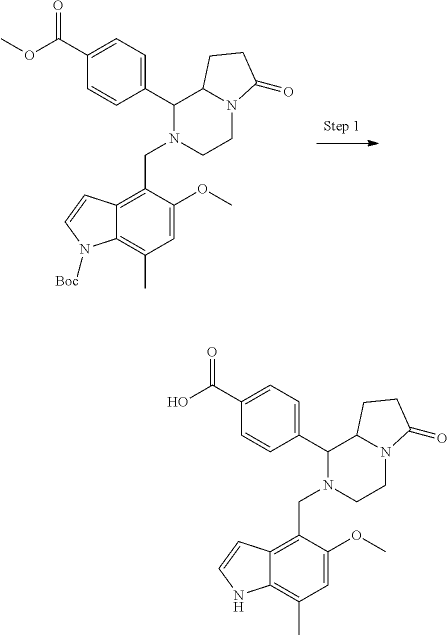 [SMILES: COC(=O)c1ccc(C2C3CCC(=O)N3CCN2Cc2c(OC)cc(C)c3c2ccn3C(=O)OC(C)(C)C)cc1 ; COc1cc(C)c2[nH]ccc2c1CN1CCN2C(=O)CCC2C1c1ccc(C(=O)O)cc1]

Step 1. 4-(2-((5-Methoxy-7-methyl-1H-indol-4-yl)methyl)-6-oxooctahydropyrrolo[1,2-a]pyrazin-1-yl)benzoic acid was prepared following General Experimental Procedure 8. tert-Butyl 5-methoxy-4-((1-(4-(methoxycarbonyl)phenyl)hexahydropyrrolo[1,2-a]pyrazin-2 (1H)-yl)methyl)-7-methyl-1H-indole-1-carboxylate (11 mg, 20 μmol) gave 4-(2-((5-methoxy-7-methyl-1H-indol-4-yl)methyl)-6-oxooctahydropyrrolo[1,2-a]pyrazin-1-yl)benzoic acid (7 mg, 80%). LCMS (31697 LCMS-5 C3) RT: min; area % (215 nm): 95%; m/z=420.2 [M+H]⁺. ¹H-NMR (400 MHz, CD₃OD) δ 8.01 (d, J=8.0 Hz, 2H), 7.35 (d, J=7.9 Hz, 2H), 7.20 (d, J=3.1 Hz, 1H), 6.70 (s, 1H), 6.54 (d, J=3.1 Hz, 1H), 4.58 (s, 1H), 4.31-4.22 (m, 1H), 4.13 (d, J=4.7 Hz, 1H), 4.05-3.98 (m, 1H), 3.91 (d, J=12.6 Hz, 1H), 3.74 (s, 3H), 3.50 (d, J=8.8 Hz, 1H), 3.09-3.00 (m, 1H), 2.89 (td, J=11.7, 4.2 Hz, 1H), 2.75-2.67 (m, 1H), 2.48 (s, 3H), 2.33-2.22 (m, 1H), 2.11-1.92 (m, 2H).

### Example 80. Diastereomer 1 of 4-((1aR,6aR,7aR)-5-((5-methoxy-7-methyl-1H-indol-4-yl)methyl)octahydro-1H-cyclopropa[4,5]pyrrolo[1,2-a]pyrazin-4-yl)benzoic acid

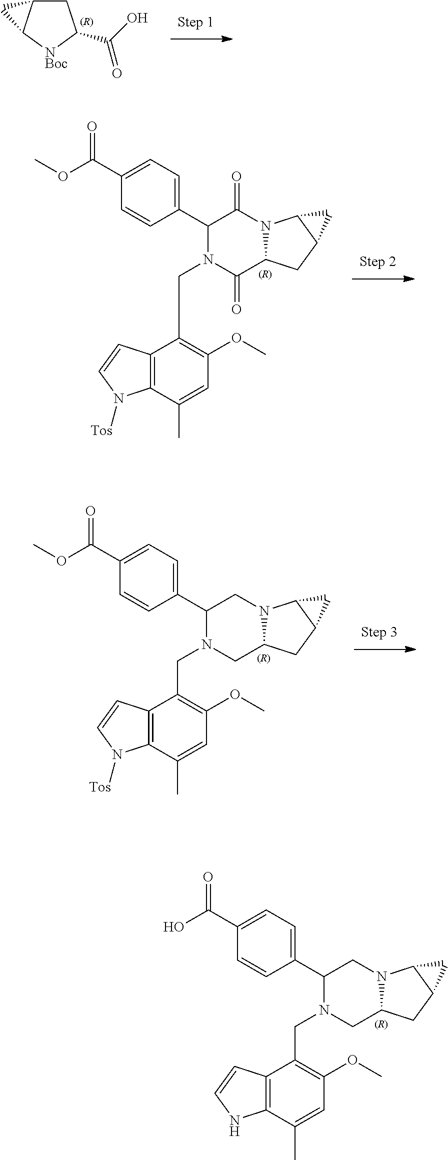

Step 1. Diastereomer 1 of methyl 4-((1aR,6aR,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate was prepared following General Experimental Procedure 9. Methyl 4-formylbenzoate (174 mg, 1.06 mmol), (5-methoxy-7-methyl-1-tosyl-1H-indol-4-yl)methanamine (350 mg, 1.06 mmol), 2-(2,2-dimehtoxyethyl)-N-methylynebenzaminium (0.19 mL, 1.06 mmol) and (1R,3R,5R)-2-(tert-butoxycarbonyl)-2-azabicyclo[3.1.0]hexane-3-carboxylic acid (241 mg, 1.06 mmol) gave diastereomer 1 of methyl 4-((1aR,6aR,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (50 mg, 7.5%). LCMS (General 3 Basic) RT: 1.77 min; area % (254 nm): 73%; m/z=628.3 [M+H]⁺.

Step 2. Diastereomer 1 of methyl 4-((1aR,6aR,7aR)-5-((5-methoxy-7-methyl-1-tosyl-1H-indol-4-yl)methyl)octahydro-1H-cyclopropa[4,5]pyrrolo[1,2-a]pyrazin-4-yl)benzoate was prepared following General Experimental Procedure 10. Diastereomer 1 of methyl 4-((1aR,6aR,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (50 mg, 80 μmol) gave diastereomer 1 of methyl 4-((1aR,6aR,7aR)-5-((5-methoxy-7-methyl-1-tosyl-1H-indol-4-yl)methyl)octahydro-1H-cyclopropa[4,5]pyrrolo[1,2-a]pyrazin-4-yl)benzoate (48 mg, 44%). LCMS (General 3 Basic) RT: 2.26 min; area % (254 nm): 44%; m/z=600.4 [M+H]⁺.

Step 3. Diastereomer 1 of 4-((1aR,6aR,7aR)-5-((5-methoxy-7-methyl-1H-indol-4-yl)methyl)octahydro-1H-cyclopropa[4,5]pyrrolo[1,2-a]pyrazin-4-yl)benzoic acid was prepared with the following procedure. To a solution of diastereomer 1 of methyl 4-((1a0,6aR,7aR)-5-((5-methoxy-7-methyl-1-tosyl-1H-indol-4-yl)methyl)octahydro-1H-cyclopropa[4,5]pyrrolo[1,2-a]pyrazin-4-yl)benzoate (48 mg, 1.0 eq, 80 μmol) in THF (2 mL) and MeOH (2 mL) was added magnesium (20 mg, 10 eq, 0.83 mmol) and NH₄Cl (8.3 mg, 2.0 eq, 0.17 mmol). The reaction mixture was stirred at 20° C. for 48 h, and then more magnesium (39 mg, 20 eq, 1.6 mmol) was added. The reaction mixture was sonicated for 40 minutes and then heated to 70° C. for 3 hours. After that, the reaction mixture was cooled to RT and lithium hydroxide monohydrate (6.7 mg, 2.0 eq, 0.16 mmol). The suspension was diluted with methanol and then filtered over Celite®. The residue was washed with methanol. The filtrates were combined and concentrated under reduced pressure. The residue was purified by automated reverse phase FCC to afford diastereomer 1 of 4-((1aR,6aR,7aR)-5-((5-methoxy-7-methyl-1H-indol-4-yl)methyl)octahydro-1H-cyclopropa[4,5]pyrrolo[1,2-a]pyrazin-4-yl)benzoic acid (1.07 mg, 3%) as a white solid. LCMS (31697 LCMS-5 C3) RT: 2.030 min; area % (215 nm): 86%; m/z=432.2 [M+H]⁺. ¹H-NMR (400 MHz, CD₃OD) δ 7.34 (d, J=8.0 Hz, 2H), 7.17-7.09 (m, 2H), 6.64 (s, 1H), 6.21 (d, J=3.1 Hz, 1H), 5.51 (s, 1H), 4.69 (dd, J=7.0, 4.5 Hz, 1H), 4.61 (d, J=2.3 Hz, 2H), 4.33-4.28 (m, 1H), 3.87-3.80 (m, 1H), 3.63 (s, 3H), 3.23 (dd, J=13.0, 7.1 Hz, 1H), 2.98 (t, J=4.7 Hz, 1H), 2.65 (td, J=6.5, 5.8, 2.7 Hz, 1H), 2.50-2.43 (m, 4H), 2.29 (ddd, J=14.1, 9.9, 5.5 Hz, 1H), 2.20 (ddd, J=13.1, 6.7, 2.0 Hz, 1H), 1.45-1.37 (m, 1H), 0.41-0.35 (m, 1H), 0.22 (q, J=6.6 Hz, 1H).

### Example 81. Diastereomer 2 4-((1aR,6aR,7aR)-5-((5-methoxy-7-methyl-1H-indol-4-yl)methyl)octahydro-1H-cyclopropa[4,5]pyrrolo[1,2-a]pyrazin-4-yl)benzoic acid

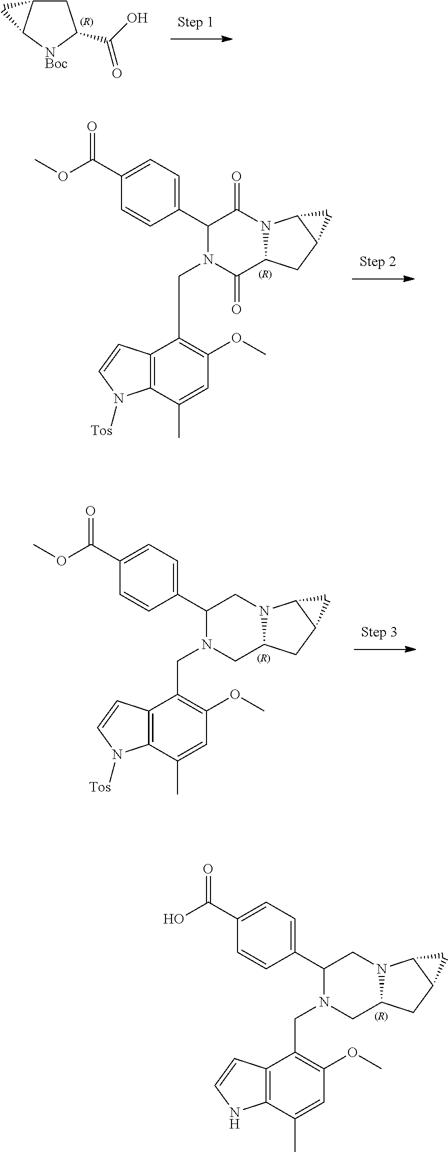

Step 1. Diastereomer 2 of methyl 4-((1aR,6aR,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate was prepared following General Experimental Procedure 9. Methyl 4-formylbenzoate (174 mg, 1.06 mmol), (5-methoxy-7-methyl-1-tosyl-1H-indol-4-yl)methanamine (350 mg, 1.06 mmol), 2-(2,2-dimehtoxyethyl)-N-methylynebenzaminium (0.19 mL, 1.06 mmol) and (1R,3R,5R)-2-(tert-butoxycarbonyl)-2-azabicyclo[3.1.0]hexane-3-carboxylic acid (241 mg, 1.06 mmol) gave diastereomer 2 of methyl 4-((1aR,6aR,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (56 mg, 8.4%). LCMS (General 3 Basic) RT: 1.82 min; area % (254 nm): 99%; m/z=628.3 [M+H]⁺.

Step 2. Diastereomer 2 of methyl 4-((1aR,6aR,7aR)-5-((5-methoxy-7-methyl-1-tosyl-1H-indol-4-yl)methyl)octahydro-1H-cyclopropa[4,5]pyrrolo[1,2-a]pyrazin-4-yl)benzoate was prepared following General Experimental Procedure 10. Diastereomer 2 of methyl 4-((1aR,6aR,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (56 mg, 89 μmol) gave diastereomer 2 of (4-((1aR,6aR,7aR)-5-((5-methoxy-7-methyl-1-tosyl-1H-indol-4-yl)methyl)octahydro-1H-cyclopropa[4,5]pyrrolo[1,2-a]pyrazin-4-yl)phenyl) methanol (48 mg, 44%). LCMS (General 3 Basic) RT: 2.00 min; area % (254 nm): 44%; m/z=572.4 [M+H]⁺.

Step 3. Diastereomer 2 4-((1aR,6aR,7aR)-5-((5-methoxy-7-methyl-1H-indol-4-yl)methyl)octahydro-1H-cyclopropa[4,5]pyrrolo[1,2-a]pyrazin-4-yl)benzoic acid was prepared with the following procedure. To a solution of diastereomer 2 of (4-((1aR,6aR,7aR)-5-((5-methoxy-7-methyl-1-tosyl-1H-indol-4-yl)methyl)octahydro-1H-cyclopropa[4,5]pyrrolo[1,2-a]pyrazin-4-yl)phenyl) methanol (48 mg, 1.0 eq, 2.4 mmol) in THF (2 mL) and MeOH (2 mL) was added ammonium chloride (8.6 mg, 2.0 eq, 0.16 mmol) and magnesium (58 mg, 30 eq, 2.4 mmol). The mixture was heated at 50° C. for 18 h. The mixture was filtered over a syringe filter and the filtrate was concentrated under reduced pressure. To the residue was added MeOH (2 mL), water (0.2 mL) and then sodium hydroxide (13 mg, 4.0 eq, 0.32 mmol) and then stirred at 20° C. After 1 h, to the mixture was added acetic acid (18 μL, 4.0 eq, 0.32 mmol) and DMSO. The suspension was filtered and purified by automated reverse phase FCC for purification to afford 4-((1aR,6aR,7aR)-5-((5-methoxy-7-methyl-1H-indol-4-yl)methyl)octahydro-1H-cyclopropa[4,5]pyrrolo[1,2-a]pyrazin-4-yl)benzoic acid (1.25 mg, 3.7%). LCMS (31697 LCMS-5 C3) RT: 2.286 min; area % (215 nm): 83%; m/z=418.2 [M+H]⁺. ¹H-NMR (400 MHz, CD₃OD) δ 7.58-7.53 (m, 2H), 7.43 (d, J=7.8 Hz, 2H), 7.19 (d, J=3.1 Hz, 1H), 6.68 (s, 1H), 6.41 (d, J=3.1 Hz, 1H), 4.65 (s, 2H), 3.85 (d, J=12.0 Hz, 1H), 3.75 (s, 3H), 3.55 (d, J=9.9 Hz, 1H), 3.39 (s, 1H), 3.24-3.10 (m, 2H), 2.89 (t, J=11.4 Hz, 1H), 2.78 (d, J=11.4 Hz, 1H), 2.55 (td, J=6.5, 5.5, 2.3 Hz, 1H), 2.45 (d, J=0.8 Hz, 3H), 2.06 (dt, J=13.1, 6.8 Hz, 2H), 1.51-1.39 (m, 1H), 1.26 (ddd, J=12.7, 7.8, 2.4 Hz, 1H), 0.86 (ddd, J=5.3, 4.3, 2.4 Hz, 1H), 0.74 (dt, J=8.8, 5.5 Hz, 1H).

### Example 82. Diastereomer 1 of 4-((6S)-4-((5-methoxy-7-methyl-1H-indol-4-yl)methyl)-1,4-diazabicyclo[4.2.0]octan-3-yl)benzoic acid

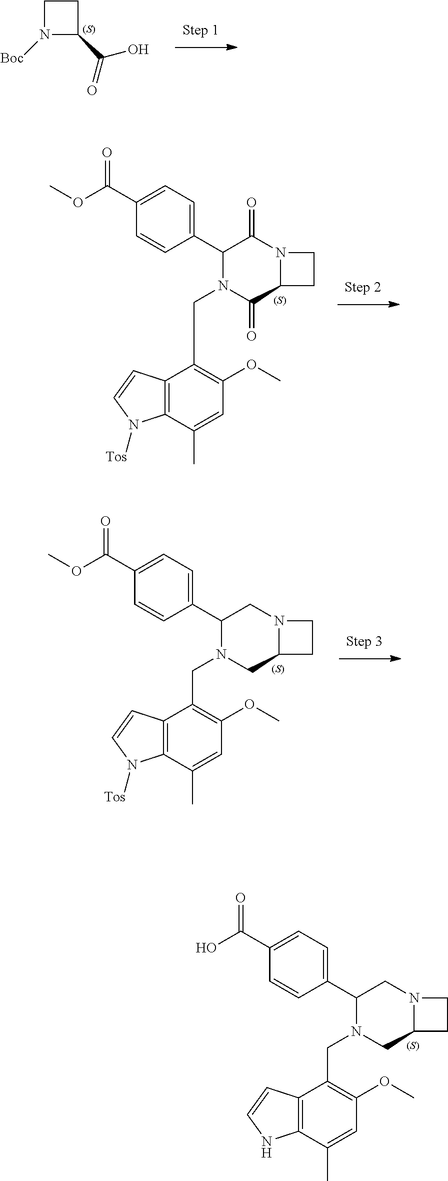

Step 1. Methyl 4-((6S)-4-((5-methoxy-7-methyl-1-tosyl-1H-indol-4-yl)methyl)-2,5-dioxo-1,4-diazabicyclo[4.2.0]octan-3-yl)benzoate was prepared following General Experimental Procedure 9. Methyl 4-formylbenzoate (286 mg, 1.74 mmol), meth (5-methoxy-7-methyl-1-tosyl-1H-indol-4-yl)methanamine (600 mg, 1.74 mmol), (S)-1-(tert-butoxycarbonyl) azetidine-2-carboxylic acid (351 mg, 1.74 mmol), and 2-(2,2-dimethoxyethyl)-N-methylidynebenzenaminium (300 μL, 1.74 mmol) gave diastereomer 1 of methyl 4-((6S)-4-((5-methoxy-7-methyl-1-tosyl-1H-indol-4-yl)methyl)-2,5-dioxo-1,4-diazabicyclo[4.2.0]octan-3-yl)benzoate (190 mg, 21%) as a yellow oil. Second eluting diastereomer LCMS (General 3 Basic) RT: 1.75 min; area % (254 nm): 66%; m/z=602.5 [M+H]⁺.

Step 2. Diastereomer 1 of methyl 4-((6S)-4-((5-methoxy-7-methyl-1-tosyl-1H-indol-4-yl)methyl)-1,4-diazabicyclo[4.2.0]octan-3-yl)benzoate was prepared following General Experimental Procedure 10. Diastereomer 1 of methyl 4-((6S)-4-((5-methoxy-7-methyl-1-tosyl-1H-indol-4-yl)methyl)-2,5-dioxo-1,4-diazabicyclo[4.2.0]octan-3-yl)benzoate (68.5 mg, 114 μmol), acetylacetonato (1,5-cyclooctadiene)rhodium (7.06 mg, 22.8 μmol) and phenylsilane (113 μL, 911 μmol) gave diastereomer 1 of methyl 4-((6S)-4-((5-methoxy-7-methyl-1-tosyl-1H-indol-4-yl)methyl)-1,4-diazabicyclo[4.2.0]octan-3-yl)benzoate (42 mg, 64%). LCMS (General 3 Basic) RT: 2.03 min; area % (254 nm): 52%; m/z=574.4 [M+H]⁺.

Step 3. Diastereomer 1 of 4-((6S)-4-((5-methoxy-7-methyl-1H-indol-4-yl)methyl)-1,4-diazabicyclo[4.2.0]octan-3-yl)benzoic acid was prepared following General Experimental Procedure 11. Diastereomer 1 of methyl 4-((6S)-4-((5-methoxy-7-methyl-1-tosyl-1H-indol-4-yl)methyl)-1,4-diazabicyclo[4.2.0]octan-3-yl)benzoate (9.0 mg, 16 μmol) gave diastereomer 1 of 4-((6S)-4-((5-methoxy-7-methyl-1H-indol-4-yl)methyl)-1,4-diazabicyclo[4.2.0]octan-3-yl)benzoic acid (3.0 mg, 47%) as a white solid. LCMS (31697 LCMS-5 C3) RT: 1.489 min; area % (215 nm): 91.8%; m/z=406.2 [M+H]⁺. ¹H-NMR (400 MHz, CD₃OD) δ 8.03 (d, J=8.3 Hz, 2H), 7.61 (d, J=8.2 Hz, 2H), 7.22 (d, J=3.1 Hz, 1H), 6.71 (s, 1H), 6.46 (d, J=3.2 Hz, 1H), 4.19 (q, J=8.7 Hz, 1H), 4.06-3.85 (m, 3H), 3.80 (s, 3H), 3.74-3.61 (m, 1H), 3.54-3.38 (m, 1H), 3.20 (dd, J=12.9, 6.1 Hz, 1H), 3.16-3.03 (m, 1H), 2.98 (d, J=14.3 Hz, 1H), 2.89-2.73 (m, 1H), 2.58-2.48 (m, 1H), 2.46 (s, 3H), 1.81 (s, 1H), 1.29 (s, 1H).

### Example 83. Diastereomer 1 of 4-((6S)-4-((5-methoxy-7-methyl-1H-indol-4-yl)methyl)-1,4-diazabicyclo[4.2.0]octan-3-yl)benzoic acid

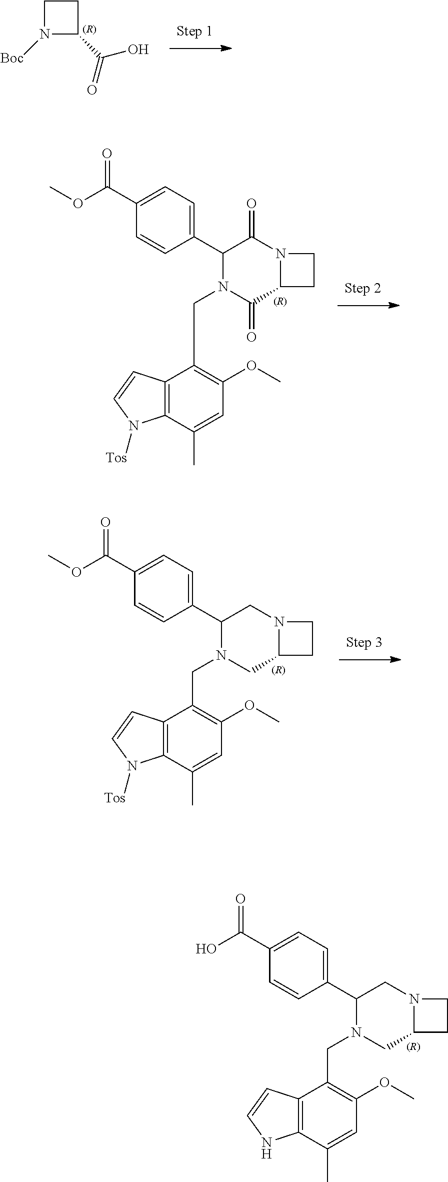

Step 1. Methyl 4-((6R)-4-((5-methoxy-7-methyl-1-tosyl-1H-indol-4-yl)methyl)-2,5-dioxo-1,4-diazabicyclo[4.2.0]octan-3-yl)benzoate was prepared following General Experimental Procedure 9. Methyl 4-formylbenzoate (238 mg, 1.45 mmol), meth (5-methoxy-7-methyl-1-tosyl-1H-indol-4-yl)methanamine (500 mg, 1.45 mmol), (R)-1-(tert-butoxycarbonyl) azetidine-2-carboxylic acid (301 mg, 1.45 mmol), and 2-(2,2-dimethoxyethyl)-N-methylidynebenzenaminium (250 μL, 1.45 mmol) gave diastereomer 1 of methyl 4-((6R)-4-((5-methoxy-7-methyl-1-tosyl-1H-indol-4-yl)methyl)-2,5-dioxo-1,4-diazabicyclo[4.2.0]octan-3-yl)benzoate (86 mg, 10%) as a yellow oil. Second eluting diastereomer LCMS (General 3 Basic) RT: 1.75 min; area % (254 nm): 89%; m/z=602.2 [M+H]⁺.

Step 2. Diastereomer 1 of methyl 4-((6R)-4-((5-methoxy-7-methyl-1-tosyl-1H-indol-4-yl)methyl)-1,4-diazabicyclo[4.2.0]octan-3-yl)benzoate was prepared following General Experimental Procedure 10. Diastereomer 1 of methyl 4-((6R)-4-((5-methoxy-7-methyl-1-tosyl-1H-indol-4-yl)methyl)-2,5-dioxo-1,4-diazabicyclo[4.2.0]octan-3-yl)benzoate (83 mg, 140 μmol), acetylacetonato (1,5-cyclooctadiene)rhodium (8.5 mg, 28 μmol) and phenylsilane (140 μL, 1.1 mmol) gave diastereomer 1 of methyl 4-((6R)-4-((5-methoxy-7-methyl-1-tosyl-1H-indol-4-yl)methyl)-1,4-diazabicyclo[4.2.0]octan-3-yl)benzoate (43 mg, 33%). LCMS (General 3 Basic) RT: 2.02 min; area % (254 nm): 60%; m/z=574.4 [M+H]⁺.

Step 3. Diastereomer 1 of 4-((6R)-4-((5-methoxy-7-methyl-1H-indol-4-yl)methyl)-1,4-diazabicyclo[4.2.0]octan-3-yl)benzoic acid was prepared following General Experimental Procedure 11. Diastereomer 1 of methyl 4-((6R)-4-((5-methoxy-7-methyl-1-tosyl-1H-indol-4-yl)methyl)-1,4-diazabicyclo[4.2.0]octan-3-yl)benzoate (42 mg, 44 μmol) gave diastereomer 1 of 4-((6R)-4-((5-methoxy-7-methyl-1H-indol-4-yl)methyl)-1,4-diazabicyclo[4.2.0]octan-3-yl)benzoic acid (2.21 mg, 11%) as a white solid. LCMS (31697 LCMS-5 C3) RT: 1.489 min; area % (215 nm): 87%; m/z=406.2 [M+H]⁺. ¹H-NMR (400 MHz, CD₃OD) δ 8.03 (d, J=8.2 Hz, 2H), 7.60 (d, J=8.3 Hz, 2H), 7.22 (d, J=3.1 Hz, 1H), 6.71 (s, 1H), 6.47 (d, J=3.1 Hz, 1H), 5.17 (s, 1H), 4.24-4.12 (m, 1H), 4.03-3.87 (m, 3H), 3.80 (s, 3H), 3.70-3.58 (m, 1H), 3.55-3.41 (m, 1H), 3.23-3.10 (m, 2H), 3.10-3.00 (m, 1H), 3.00-2.87 (m, 1H), 2.87-2.71 (m, 1H), 2.51 (d, J=10.6 Hz, 1H), 2.46 (s, 3H), 1.86-1.71 (m, 1H).

### Example 84. Diastereomer 1 of 4-((7S,8aR)-7-fluoro-2-((5-methoxy-7-methyl-1H-indol-4-yl)methyl)octahydropyrrolo[1,2-a]pyrazin-3-yl)benzoic acid

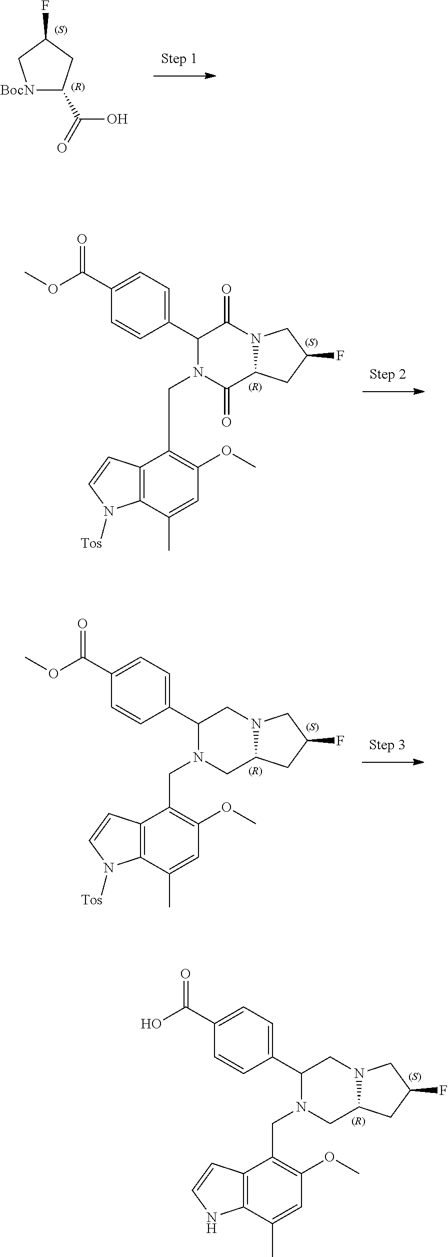

Step 1. Methyl 4-((7S,8aR)-7-fluoro-2-((5-methoxy-7-methyl-1-tosyl-1H-indol-4-yl)methyl)-1,4-dioxooctahydropyrrolo[1,2-a]pyrazin-3-yl)benzoate was prepared following General Experimental Procedure 9. Methyl 4-formylbenzoate (238 mg, 1.45 mmol), (5-methoxy-7-methyl-1-tosyl-1H-indol-4-yl)methanamine (500 mg, 1.45 mmol), (2R,4S)-1-(tert-butoxycarbonyl)-4-fluoropyrrolidine-2-carboxylic acid (339 mg, 1.45 mmol), and 2-(2,2-dimethoxyethyl)-N-methylidynebenzenaminium (250 μL, 1.45 mmol) gave diastereomer 1 of methyl 4-((7S,8aR)-7-fluoro-2-((5-methoxy-7-methyl-1-tosyl-1H-indol-4-yl)methyl)-1,4-dioxooctahydropyrrolo[1,2-a]pyrazin-3-yl)benzoate (115 mg, 13%) as a green oil. LCMS (General 3 Basic) RT: 1.78 min and 1.81 min; area % combined (254 nm): 88%; m/z=634.4 [M+H]⁺.

Step 2. Methyl 4-((7S,8aR)-7-fluoro-2-((5-methoxy-7-methyl-1-tosyl-1H-indol-4-yl)methyl)octahydropyrrolo[1,2-a]pyrazin-3-yl)benzoate was prepared following General Experimental Procedure 10. Methyl 4-((7S,8aR)-7-fluoro-2-((5-methoxy-7-methyl-1-tosyl-1H-indol-4-yl)methyl)-1,4-dioxooctahydropyrrolo[1,2-a]pyrazin-3-yl)benzoate (115 mg, 181 μmol) gave methyl 4-((7S,8aR)-7-fluoro-2-((5-methoxy-7-methyl-1-tosyl-1H-indol-4-yl)methyl)octahydropyrrolo[1,2-a]pyrazin-3-yl)benzoate (117 mg, 106%). LCMS (General 3 Basic) RT: 2.15 min; area % (254 nm): 67%; m/z=606.5 [M+H]⁺.

Step 3. Diastereomer 1 of 4-((7S,8aR)-7-fluoro-2-((5-methoxy-7-methyl-1H-indol-4-yl)methyl)octahydropyrrolo[1,2-a]pyrazin-3-yl)benzoic acid was prepared following General Experimental Procedure 11. Methyl 4-((7S,8aR)-7-fluoro-2-((5-methoxy-7-methyl-1-tosyl-1H-indol-4-yl)methyl)octahydropyrrolo[1,2-a]pyrazin-3-yl)benzoate (117 mg, 193 μmol) gave diastereomer 1 of 4-((7S,8aR)-7-fluoro-2-((5-methoxy-7-methyl-1H-indol-4-yl)methyl)octahydropyrrolo[1,2-a]pyrazin-3-yl)benzoic acid (7.48 mg, 9%) as a white solid. LCMS (31697 LCMS-5 C3) RT: 1.769 min; area % (215 nm): 91.8%; m/z=438.2 [M+H]⁺. ¹H NMR (400 MHz, CD₃OD) δ 8.09 (d, 2H), 7.67 (d, J=7.8 Hz, 2H), 7.25 (d, J=3.1 Hz, 1H), 6.72 (s, 1H), 6.39 (d, J=3.1 Hz, 1H), 5.33-5.10 (m, 3H), 4.58 (s, 1H), 4.05 (s, 3H), 3.76 (s, 3H), 3.59 (ddd, J=17.0, 10.6, 6.0 Hz, 2H), 3.24 (s, 1H), 2.68 (d, J=21.2 Hz, 3H), 2.52 (s, 1H), 2.48 (d, J=0.8 Hz, 3H), 2.43 (d, J=10.7 Hz, 1H), 2.05-1.89 (m, 2H), 1.80-1.62 (m, 2H). 19F-NMR (376 MHz, CD₃OD) δ −76.94.

### Example 85. Diastereomer 2 of 4-((7S,8aR)-7-fluoro-2-((5-methoxy-7-methyl-1H-indol-4-yl)methyl)octahydropyrrolo[1,2-a]pyrazin-3-yl)benzoic acid

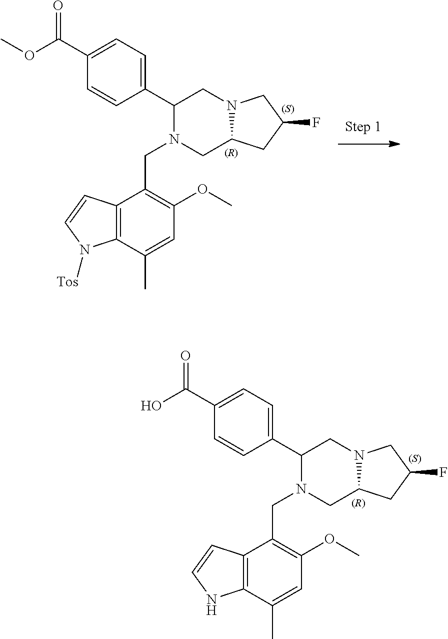

Step 1. Diastereomer 2 of 4-((7S,8aR)-7-fluoro-2-((5-methoxy-7-methyl-1H-indol-4-yl)methyl)octahydropyrrolo[1,2-a]pyrazin-3-yl)benzoic acid was prepared following General Experimental Procedure 11. Methyl 4-((7S,8aR)-7-fluoro-2-((5-methoxy-7-methyl-1-tosyl-1H-indol-4-yl)methyl)octahydropyrrolo[1,2-a]pyrazin-3-yl)benzoate (117 mg, 193 μmol) gave diastereomer 2 of 4-((7S,8aR)-7-fluoro-2-((5-methoxy-7-methyl-1H-indol-4-yl)methyl)octahydropyrrolo[1,2-a]pyrazin-3-yl)benzoic acid (6.67 mg, 8%) as a white solid. LCMS (31697 LCMS-5 C3) RT: 1.770 min; area % (215 nm): 93.0%; m/z=438.2 [M+H]⁺. ¹H-NMR (400 MHz, CD₃OD) δ 8.01 (d, 2H), 7.68 (d, J=8.1 Hz, 2H), 7.23 (d, J=3.1 Hz, 1H), 6.71 (s, 1H), 6.50 (d, J=3.1 Hz, 1H), 5.51-5.22 (m, 3H), 4.58 (s, 1H), 3.81 (s, 3H), 3.77-3.56 (m, 3H), 3.46-3.36 (m, 3H), 2.99 (s, 3H), 2.81 (s, 1H), 2.48 (s, 3H), 2.45-2.27 (m, 2H), 1.95-1.82 (m, 2H). 19F-NMR (376 MHz, CD₃OD) δ −76.94.

### Example 86. Diastereomer 1 of 4-((9aR)-2-((5-methoxy-7-methyl-1H-indol-4-yl)methyl)octahydro-2H-pyrido[1,2-a]pyrazin-3-yl)benzoic acid

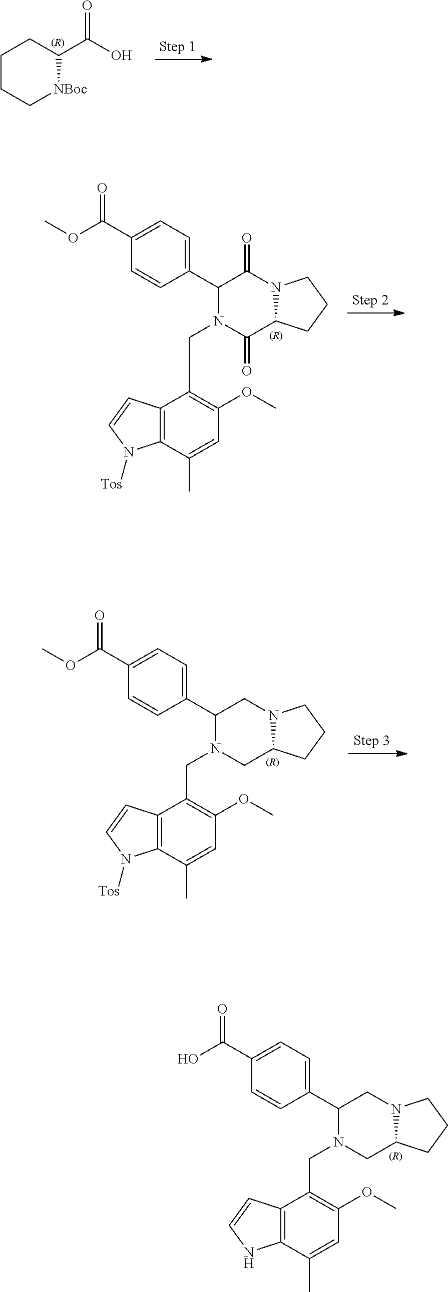

Step 1. Methyl 4-((9aR)-2-((5-methoxy-7-methyl-1-tosyl-1H-indol-4-yl)methyl)-1,4-dioxooctahydro-2H-pyrido[1,2-a]pyrazin-3-yl)benzoate was prepared following General Experimental Procedure 9. Methyl 4-formylbenzoate (180 mg, 1.10 mmol), (5-methoxy-7-methyl-1-tosyl-1H-indol-4-yl)methanamine (378 mg, 1.10 mmol), (R)-1-(tert-butoxycarbonyl) piperidine-2-carboxylic acid (251 mg, 1.10 mmol), and 2-(2,2-dimethoxyethyl)-N-methylidynebenzenaminium (190 μL, 1.10 mmol) gave diastereomer 1 of methyl 4-((9aR)-2-((5-methoxy-7-methyl-1-tosyl-1H-indol-4-yl)methyl)-1,4-dioxooctahydro-2H-pyrido[1,2-a]pyrazin-3-yl)benzoate (178 mg, 26%) as a yellow oil. LCMS (General 3 Basic) RT: 1.87 min and 1.89 min; area % combined (254 nm): 84%; m/z=630.5 [M+H]⁺.

Step 2. Methyl 4-((9aR)-2-((5-methoxy-7-methyl-1-tosyl-1H-indol-4-yl)methyl)octahydro-2H-pyrido[1,2-a]pyrazin-3-yl)benzoate was prepared following General Experimental Procedure 10. Methyl 4-((9aR)-2-((5-methoxy-7-methyl-1-tosyl-1H-indol-4-yl)methyl)-1,4-dioxooctahydro-2H-pyrido[1,2-a]pyrazin-3-yl)benzoate (170 mg, 270 μmol) gave methyl 4-((9aR)-2-((5-methoxy-7-methyl-1-tosyl-1H-indol-4-yl)methyl)octahydro-2H-pyrido[1,2-a]pyrazin-3-yl)benzoate (150 mg, 93%). LCMS (General 3 Basic) RT: 2.15 min and 2.40; area % combined (254 nm): 62%; m/z=602.4 [M+H]⁺.

Step 3. Diastereomer 1 of 4-((9aR)-2-((5-methoxy-7-methyl-1H-indol-4-yl)methyl)octahydro-2H-pyrido[1,2-a]pyrazin-3-yl)benzoic acid was prepared following General Experimental Procedure 11. Methyl 4-((9aR)-2-((5-methoxy-7-methyl-1-tosyl-1H-indol-4-yl)methyl)octahydro-2H-pyrido[1,2-a]pyrazin-3-yl)benzoate (140 mg, 233 μmol) gave diastereomer 1 of 4-((9aR)-2-((5-methoxy-7-methyl-1H-indol-4-yl)methyl)octahydro-2H-pyrido[1,2-a]pyrazin-3-yl)benzoic acid (1.28 mg, 1%) as a white solid. LCMS (31697 LCMS-5 C3) RT: 1.669 min; area % (215 nm): 97.2%; m/z=434.2 [M+H]⁺. ¹H NMR (400 MHz, CD₃OD) δ 8.07 (d, 2H), 7.65 (d, J=7.8 Hz, 2H), 7.23 (d, J=3.1 Hz, 1H), 6.71 (s, 1H), 6.40 (d, J=3.1 Hz, 1H), 4.58 (s, 1H), 3.90 (d, J=12.0 Hz, 1H), 3.82 (s, 1H), 3.77 (s, 3H), 3.52 (s, 1H), 3.07 (d, J=12.4 Hz, 2H), 2.96 (d, J=12.3 Hz, 1H), 2.80 (t, J=11.8 Hz, 1H), 2.60 (s, 1H), 2.47 (s, 3H), 2.45-2.35 (m, 2H), 1.77 (d, J=12.6 Hz, 2H), 1.66 (q, J=13.3 Hz, 1H), 1.55 (d, J=12.4 Hz, 1H), 1.43-1.24 (m, 3H).

### Example 87. Diastereomer 2 of 4-((9aR)-2-((5-methoxy-7-methyl-1H-indol-4-yl)methyl)octahydro-2H-pyrido[1,2-a]pyrazin-3-yl)benzoic acid

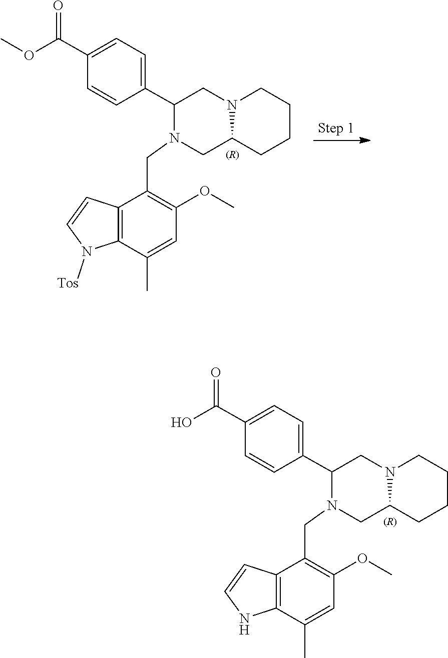

Step 1. Diastereomer 2 of 4-((9aR)-2-((5-methoxy-7-methyl-1H-indol-4-yl)methyl)octahydro-2H-pyrido[1,2-a]pyrazin-3-yl)benzoic acid was prepared following General Experimental Procedure 11. Methyl 4-((9aR)-2-((5-methoxy-7-methyl-1-tosyl-1H-indol-4-yl)methyl)octahydro-2H-pyrido[1,2-a]pyrazin-3-yl)benzoate (140 mg, 233 μmol) gave diastereomer 2 of 4-((9aR)-2-((5-methoxy-7-methyl-1H-indol-4-yl)methyl)octahydro-2H-pyrido[1,2-a]pyrazin-3-yl)benzoic acid (1.31 mg, 1%) as a white solid. LCMS (31697 LCMS-5 C3) RT: 1.673 min; area % (215 nm): 98.8%; m/z=434.2 [M+H]⁺. ¹H NMR (400 MHz, CD₃OD) δ 8.02 (d, J=7.9 Hz, 2H), 7.73 (s, 2H), 7.23 (s, 1H), 6.71 (s, 1H), 6.51 (d, J=3.1 Hz, 1H), 4.58 (s, 2H), 3.81 (s, 3H), 3.62-3.46 (m, 2H), 3.04 (s, 1H), 2.85 (d, J=13.2 Hz, 1H), 2.62 (s, 1H), 2.47 (s, 3H), 1.84 (d, J=12.8 Hz, 2H), 1.68-1.58 (m, 2H), 1.46 (d, J=13.2 Hz, 1H), 1.37-1.24 (m, 2H).

### Example 88. Diastereomer 1 of 4-((1aR,6aS,7aR)-5-((5-methoxy-7-methyl-1H-indol-4-yl)methyl)octahydro-1H-cyclopropa[4,5]pyrrolo[1,2-a]pyrazin-4-yl)benzoic acid

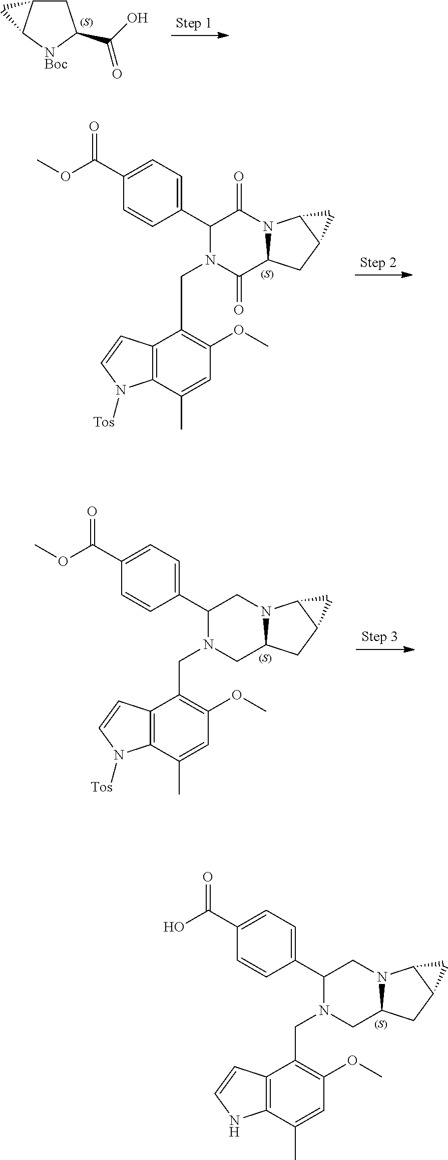

Step 1. Diastereomer 1 of methyl 4-((1aR,6aS,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate was prepared following General Experimental Procedure 9. Methyl 4-formylbenzoate (221 mg, 1.34 mmol), (5-methoxy-7-methyl-1-tosyl-1H-indol-4-yl) methanamine (444 mg, 1.34 mmol), 2-(2,2-dimehtoxyethyl)-N-methylynebenzaminium (0.23 mL, 1.34 mmol) and (1R,3S,5R)-2-(tert-butoxycarbonyl)-2-azabicyclo[3.1.0]hexane-3-carboxylic acid (305 mg, 1.34 mmol) gave diastereomer 1 of methyl 4-((1aR,6aS,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (120 mg, 14.4%). LCMS (General 3 Basic) RT: 1.80 min; area % (254 nm): 74%; m/z=628.4 [M+H]⁺.

Step 2. Diastereomer 1 of methyl 4-((1aR,6aS,7aR)-5-((5-methoxy-7-methyl-1-tosyl-1H-indol-4-yl)methyl)-3-oxooctahydro-1H-cyclopropa[4,5]pyrrolo[1,2-a]pyrazin-4-yl)benzoate was prepared following General Experimental Procedure 10. diastereomer 1 of methyl 4-((1aR,6aS,7aR)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (120 mg, 191 mmol) gave diastereomer 1 of methyl 4-((1aR,6aS,7aR)-5-((5-methoxy-7-methyl-1-tosyl-1H-indol-4-yl)methyl)-3-oxooctahydro-1H-cyclopropa[4,5]pyrrolo[1,2-a]pyrazin-4-yl)benzoate (110 mg, 44%). LCMS (General 3 Basic) RT: 2.17 min; area % (254 nm): 43%; m/z=600.4 [M+H]⁺.

Step 3. Diastereomer 1 4-((1aR,6aS,7aR)-5-((5-methoxy-7-methyl-1H-indol-4-yl)methyl)octahydro-1H-cyclopropa[4,5]pyrrolo[1,2-a]pyrazin-4-yl)benzoic acid was prepared following General Experimental Procedure 11, with the exception that lithium hydroxide monohydrate (7.7 mg, 2.0 eq, 0.18 mmol) was used instead of 1.0 M aq. NaOH. Diastereomer 1 of methyl 4-((1aR,6aS,7aR)-5-((5-methoxy-7-methyl-1-tosyl-1H-indol-4-yl)methyl)-3-oxooctahydro-1H-cyclopropa[4,5]pyrrolo[1,2-a]pyrazin-4-yl)benzoate (55 mg, 92 μmol) gave diastereomer 1 4-((1aR,6aS,7aR)-5-((5-methoxy-7-methyl-1H-indol-4-yl)methyl)octahydro-1H-cyclopropa[4,5]pyrrolo[1,2-a]pyrazin-4-yl)benzoic acid (1.1 mg, 2.8%). LCMS (31697 LCMS-5 C3) RT: 1.783 min; area % (215 nm): 79%; m/z=432.2 [M+H^{]+}. ¹H-NMR (400 MHz, CD₃OD) δ 8.07 (d, J=7.8 Hz, 2H), 7.62 (s, 1H), 7.20-7.15 (m, 1H), 6.67 (s, 1H), 6.37 (d, J=3.2 Hz, 1H), 4.58 (s, 2H), 3.79-3.71 (m, 4H), 3.13 (s, 2H), 2.72 (s, 1H), 2.45 (s, 3H), 2.19 (s, 1H), 1.75 (s, 2H), 1.54 (s, 2H), 1.31 (d, J=18.7 Hz, 2H), 0.98 (s, 1H), 0.90 (s, 1H).

### Example 89. Diastereomer 2 of 4-((9aS)-2-((5-methoxy-7-methyl-1H-indol-4-yl)methyl)octahydro-2H-pyrido[1,2-a]pyrazin-3-yl)benzoic acid

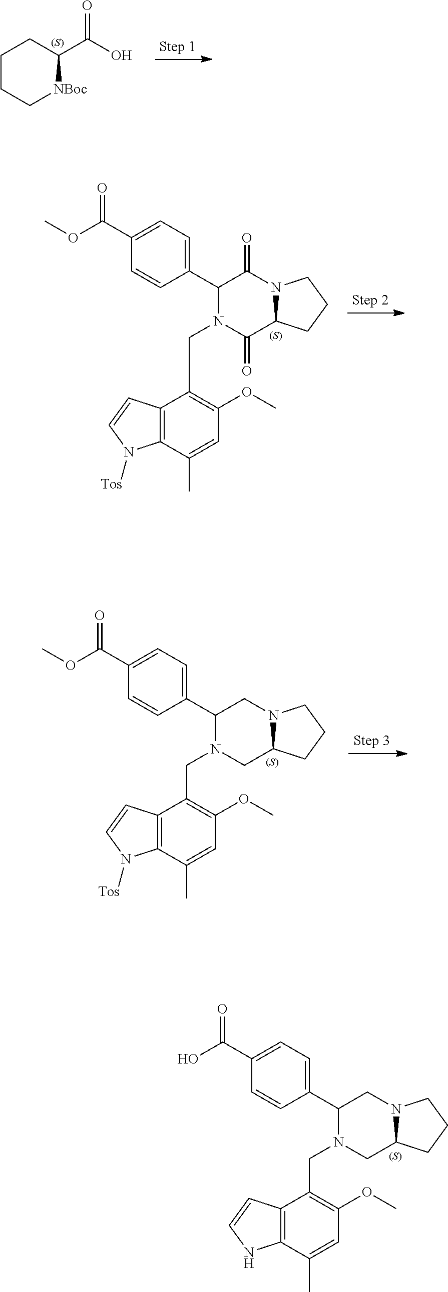

Step1. Methyl 4-((9aS)-2-((5-methoxy-7-methyl-1-tosyl-1H-indol-4-yl)methyl)-1,4-dioxooctahydro-2H-pyrido[1,2-a]pyrazin-3-yl)benzoate was prepared following General Experimental Procedure 9. Methyl 4-formylbenzoate (357 mg, 2.18 mmol), (5-methoxy-7-methyl-1-tosyl-1H-indol-4-yl) methanamine (750 mg, 2.18 mmol), (S)-1-(tert-butoxycarbonyl) piperidine-2-carboxylic acid (499 mg, 2.18 mmol), and 2-(2,2-dimethoxyethyl)-N-methylidynebenzenaminium (380 μL, 2.18 mmol) gave diastereomer 2 of methyl 4-((9aS)-2-((5-methoxy-7-methyl-1-tosyl-1H-indol-4-yl)methyl)-1,4-dioxooctahydro-2H-pyrido[1,2-a]pyrazin-3-yl)benzoate (223 mg, 16%) as a yellow oil. LCMS (General 3 Basic) RT: 1.87 min and 1.90 min; area % combined (254 nm): 81%; m/z=630.6 [M+H]⁺.

Step2. Methyl 4-((9aS)-2-((5-methoxy-7-methyl-1-tosyl-1H-indol-4-yl)methyl)octahydro-2H-pyrido[1,2-a]pyrazin-3-yl)benzoate was prepared following General Experimental Procedure 10. Methyl 4-((9aS)-2-((5-methoxy-7-methyl-1-tosyl-1H-indol-4-yl)methyl)-1,4-dioxooctahydro-2H-pyrido[1,2-a]pyrazin-3-yl)benzoate (223 mg, 354 μmol) gave methyl 4-((9aS)-2-((5-methoxy-7-methyl-1-tosyl-1H-indol-4-yl)methyl)octahydro-2H-pyrido[1,2-a]pyrazin-3-yl)benzoate (225 mg, 106%). LCMS (General 3 Basic) RT: 2.19 min and 2.44; area % combined (254 nm): 64%; m/z=602.5 [M+H]⁺.

Step 3. Diastereomer 2 of 4-((9aS)-2-((5-methoxy-7-methyl-1H-indol-4-yl)methyl)octahydro-2H-pyrido[1,2-a]pyrazin-3-yl)benzoic acid was prepared following General Experimental Procedure 11. Methyl 4-((9aS)-2-((5-methoxy-7-methyl-1-tosyl-1H-indol-4-yl)methyl)octahydro-2H-pyrido[1,2-a]pyrazin-3-yl)benzoate (215 mg, 355 μmol) gave diastereomer 2 of 4-((9aS)-2-((5-methoxy-7-methyl-1H-indol-4-yl)methyl)octahydro-2H-pyrido[1,2-a]pyrazin-3-yl)benzoic acid (5.29 mg, 3%) as a white solid. LCMS (31697 LCMS-5 C3) RT: 1.660 min; area % (215 nm): 97.9%; m/z=434.3 [M+H]⁺. ¹H NMR (400 MHz, CD₃OD) δ 8.03 (d, J=7.9 Hz, 2H), 7.73 (s, 2H), 7.23 (d, J=3.1 Hz, 1H), 6.71 (s, 1H), 6.51 (d, J=3.2 Hz, 1H), 4.58 (s, OH), 3.81 (s, 3H), 3.56 (s, 1H), 3.21-3.12 (m, 2H), 3.08 (d, J=12.5 Hz, 2H), 2.87 (d, J=13.1 Hz, 1H), 2.64 (s, 1H), 2.47 (s, 3H), 1.85 (d, J=13.0 Hz, 2H), 1.64 (d, J=14.0 Hz, 1H), 1.47 (d, J=13.5 Hz, 1H), 1.41-1.28 (m, 2H).

### Example 90. 4-((7R,8aS)-7-Fluoro-2-((5-methoxy-7-methyl-1H-indol-4-yl)methyl)octahydropyrrolo[1,2-a]pyrazin-3-yl)benzoic acid

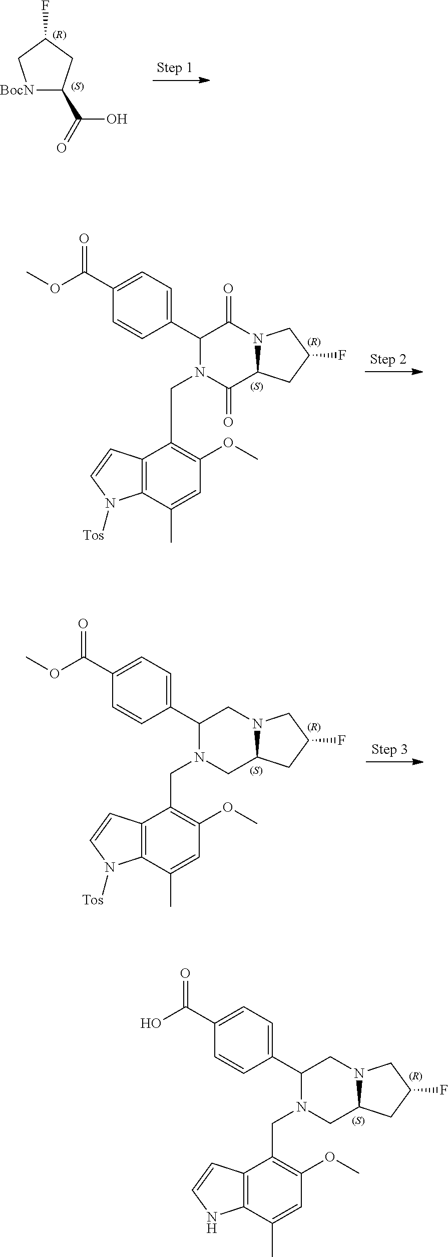

Step 1. Methyl 4-((7R,8aS)-7-fluoro-2-((5-methoxy-7-methyl-1-tosyl-1H-indol-4-yl)methyl)-1,4-dioxooctahydropyrrolo[1,2-a]pyrazin-3-yl)benzoate was prepared following General Experimental Procedure 9. Methyl 4-formylbenzoate (238 mg, 1.45 mmol), (5-methoxy-7-methyl-1-tosyl-1H-indol-4-yl) methanamine (500 mg, 1.45 mmol), (2S,4R)-1-(tert-butoxycarbonyl)-4-fluoropyrrolidine-2-carboxylic acid (339 mg, 1.45 mmol), and 2-(2,2-dimethoxyethyl)-N-methylidynebenzenaminium (250 μL, 1.45 mmol) gave diastereomer 1 of methyl 4-((7R,8aS)-7-fluoro-2-((5-methoxy-7-methyl-1-tosyl-1H-indol-4-yl)methyl)-1,4-dioxooctahydropyrrolo[1,2-a]pyrazin-3-yl)benzoate (180 mg, 20%) as a yellow oil. LCMS (General 3 Basic) RT: 1.76 min and 1.79 min; area % combined (254 nm): 72%; m/z=634.2 [M+H]⁺.

Step 2. Methyl 4-((7R,8aS)-7-fluoro-2-((5-methoxy-7-methyl-1-tosyl-1H-indol-4-yl)methyl)octahydropyrrolo[1,2-a]pyrazin-3-yl)benzoate was prepared following General Experimental Procedure 10. Methyl 4-((7R,8aS)-7-fluoro-2-((5-methoxy-7-methyl-1-tosyl-1H-indol-4-yl)methyl)-1,4-dioxooctahydropyrrolo[1,2-a]pyrazin-3-yl)benzoate (180 mg, 284 μmol) gave methyl 4-((7R,8aS)-7-fluoro-2-((5-methoxy-7-methyl-1-tosyl-1H-indol-4-yl)methyl)octahydropyrrolo[1,2-a]pyrazin-3-yl)benzoate (180 mg, 105%). LCMS (General 3 Basic) RT: 2.13 min; area % (254 nm): 51%; m/z=606.3 [M+H]⁺.

Step 3. 4-((7R,8aS)-7-Fluoro-2-((5-methoxy-7-methyl-1H-indol-4-yl)methyl)octahydropyrrolo[1,2-a]pyrazin-3-yl)benzoic acid was prepared following General Experimental Procedure 11. Methyl 4-((7R,8aS)-7-fluoro-2-((5-methoxy-7-methyl-1-tosyl-1H-indol-4-yl)methyl)octahydropyrrolo[1,2-a]pyrazin-3-yl)benzoate (105 mg, 173 μmol) gave 4-((7R,8aS)-7-fluoro-2-((5-methoxy-7-methyl-1H-indol-4-yl)methyl)octahydropyrrolo[1,2-a]pyrazin-3-yl)benzoic acid (4.46 mg, 6%) as a white solid. LCMS (31697 LCMS-5 C3) RT: 1.746 min and 1.767 min; area % combined (215 nm): 87.9%; m/z=438.2 [M+H]⁺. ¹H NMR (400 MHz, CD₃OD) δ 8.08 (d, J=8.5 Hz, 1H), 8.01 (d, 2H), 7.70-7.59 (m, 3H), 7.26-7.20 (m, 1H), 6.71 (s, 2H), 6.51 (d, J=3.1 Hz, 1H), 6.39 (d, J=3.2 Hz, 1H), 5.41 (s, 1H), 5.27 (s, 1H), 4.00 (s, 1H), 3.80 (s, 3H), 3.76 (s, 1H), 3.69 (s, 1H), 3.65-3.53 (m, 2H), 3.22-3.15 (m, 1H), 3.12-3.02 (m, 1H), 2.98 (d, J=4.7 Hz, 1H), 2.97-2.91 (m, 2H), 2.78 (d, J=12.6 Hz, 1H), 2.71-2.63 (m, 1H), 2.47 (s, 4H), 2.44-2.38 (m, 1H), 2.01-1.64 (m, 3H). ¹⁹F NMR (376 MHz, CD₃OD) δ −171.63, −172.93.

### Example 91. Diastereomer 1 of 4-((1aS,6aS,7aS)-5-((5-methoxy-7-methyl-1H-indol-4-yl)methyl)octahydro-1H-cyclopropa[4,5]pyrrolo[1,2-a]pyrazin-4-yl)benzoic acid

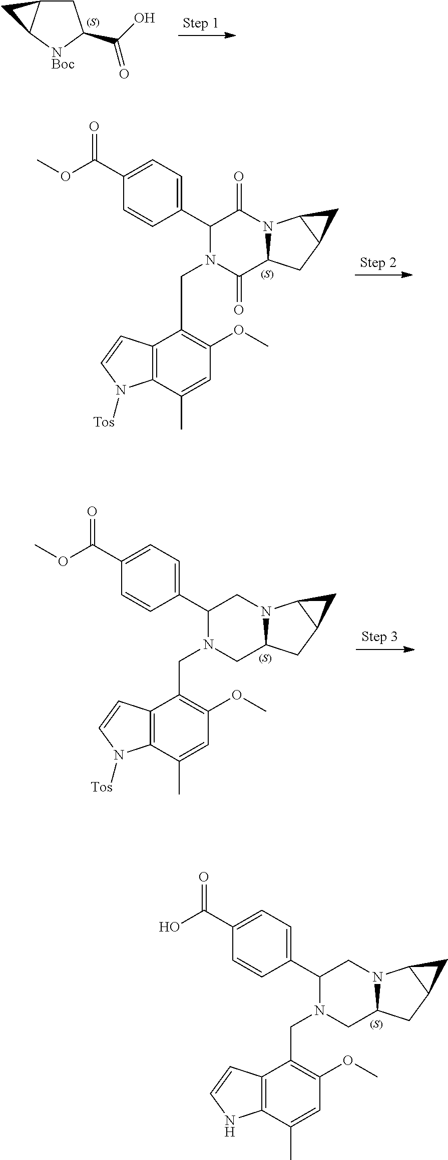

Step 1. Methyl 4-((1aS,6aS,7aS)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate was prepared following General Experimental Procedure 9. Methyl 4-formylbenzoate (248 mg, 1.51 mmol), (5-methoxy-7-methyl-1-tosyl-1H-indol-4-yl) methanamine (500 mg, 1.51 mmol), 2-(2,2-dimehtoxyethyl)-N-methylynebenzaminium (0.26 mL, 1.51 mmol) and (1S,3S,5S)-2-[(tert-butoxy)carbonyl]-2-azabicyclo[3.1.0]hexane-3-carboxylic acid (344 mg, 1.51 mmol) gave methyl 4-((1aS,6aS,7aS)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (100 mg, 10.5%). LCMS (General 3 Basic) RT: 1.77 min and 1.82 min; combined area % (254 nm): 99%; m/z=628.3 [M+H]⁺.

Step 2. Methyl 4-((1aS,6aS,7aS)-5-((5-methoxy-7-methyl-1-tosyl-1H-indol-4-yl)methyl)octahydro-1H-cyclopropa[4,5]pyrrolo[1,2-a]pyrazin-4-yl)benzoate was prepared following General Experimental Procedure 10. Methyl 4-((1aS,6aS,7aS)-5-((5-methoxy-7-methyl-1-tosyl-1H-indol-4-yl)methyl)-3,6-dioxooctahydro-1H-cyclopropa[4,5]pyrrolo[1,2-a]pyrazin-4-yl)benzoate (100 mg, 0.159 mmol), gave methyl 4-((1aS,6aS,7aS)-5-((5-methoxy-7-methyl-1-tosyl-1H-indol-4-yl)methyl)octahydro-1H-cyclopropa[4,5]pyrrolo[1,2-a]pyrazin-4-yl)benzoate (71 mg, 74%). LCMS (General 3 Basic) RT: 2.24 min and 2.31 min; combined area % (254 nm): 60%; m/z=586.4 [M+H−Me]⁺.

Step 3. Diastereomer 1 of 4-((1aS,6aS,7aS)-5-((5-methoxy-7-methyl-1H-indol-4-yl)methyl)octahydro-1H-cyclopropa[4,5]pyrrolo[1,2-a]pyrazin-4-yl)benzoic acid was prepared following General Experimental Procedure 11, with the exception that lithium hydroxide monohydrate (39 mg, 7.1 eq, 0.94 mmol) was used instead of 1.0 M aq. NaOH. Methyl 4-((1aS,6aS,7aS)-5-((5-methoxy-7-methyl-1-tosyl-1H-indol-4-yl)methyl)octahydro-1H-cyclopropa[4,5]pyrrolo[1,2-a]pyrazin-4-yl)benzoate (79 mg, 0.13 mmol) gave diastereomer 1 of 4-((1aS,6aS,7aS)-5-((5-methoxy-7-methyl-1H-indol-4-yl)methyl)octahydro-1H-cyclopropa[4,5]pyrrolo[1,2-a]pyrazin-4-yl)benzoic acid (1.64 mg, 2.9%). LCMS (31697 LCMS-5 C3) RT: 1.699 min; area % (215 nm): 58%; m/z=432.2 [M+H]⁺. ¹H-NMR (400 MHz, CD₃OD) δ 8.04 (d, J=8.1 Hz, 2H), 7.61 (d, J=7.5 Hz, 2H), 7.21 (d, J=3.0 Hz, 1H), 6.69 (s, 1H), 6.16 (d, J=3.1 Hz, 1H), 4.58 (s, 2H), 4.20 (s, 2H), 3.77-3.71 (m, 4H), 3.13-3.08 (m, 1H), 2.51-2.45 (m, 4H), 2.25-2.15 (m, 2H), 2.03 (s, 1H), 1.66 (d, J=12.6 Hz, 2H), 0.92-0.68 (m, 2H).

### Example 92. Diastereomer 2 of 4-((1aS,6aS,7aS)-5-((5-methoxy-7-methyl-1H-indol-4-yl)methyl)octahydro-1H-cyclopropa[4,5]pyrrolo[1,2-a]pyrazin-4-yl)benzoic acid

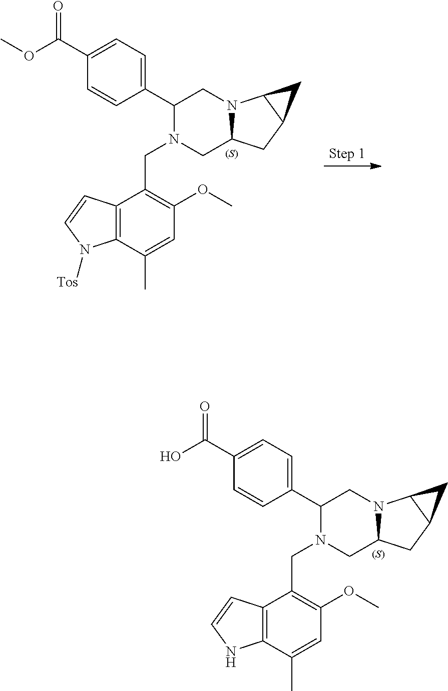

Step 1. Diastereomer 2 of 4-((1aS,6aS,7aS)-5-((5-methoxy-7-methyl-1H-indol-4-yl)methyl)octahydro-1H-cyclopropa[4,5]pyrrolo[1,2-a]pyrazin-4-yl)benzoic acid was prepared following General Experimental Procedure 11, with the exception that lithium hydroxide monohydrate (39 mg, 7.1 eq, 0.94 mmol) was used instead of 1.0 M aq. NaOH. Methyl 4-((1aS,6aS,7aS)-5-((5-methoxy-7-methyl-1-tosyl-1H-indol-4-yl)methyl)octahydro-1H-cyclopropa[4,5]pyrrolo[1,2-a]pyrazin-4-yl)benzoate (79 mg, 0.13 mmol) gave diastereomer 2 of 4-((1aS,6aS,7aS)-5-((5-methoxy-7-methyl-1H-indol-4-yl)methyl)octahydro-1H-cyclopropa[4,5]pyrrolo[1,2-a]pyrazin-4-yl)benzoic acid. LCMS (31697 LCMS-5 C3) RT: 1.699 min; area % (215 nm): 58%; m/z=432.2 [M+H]⁺. ¹H-NMR (400 MHz, CD₃OD) δ 8.14 (d, J=8.1 Hz, 2H), 7.68 (d, J=8.3 Hz, 2H), 7.28 (d, J=3.1 Hz, 1H), 6.74 (s, 1H), 6.34 (d, J=3.1 Hz, 1H), 4.36-4.12 (m, 2H), 3.99-3.88 (m, 1H), 3.75 (s, 3H), 3.44 (d, J=3.7 Hz, 2H), 3.18 (s, 1H), 2.75 (s, 1H), 2.49 (s, 3H), 2.20 (dt, J=13.0, 6.9 Hz, 2H), 1.57 (d, J=7.0 Hz, 1H), 1.42-1.31 (m, 2H), 0.92-0.84 (m, 2H).

### Example 93. 4-((7R,8aR)-7-Fluoro-2-((5-methoxy-7-methyl-1H-indol-4-yl)methyl)octahydropyrrolo[1,2-a]pyrazin-3-yl)benzoic acid

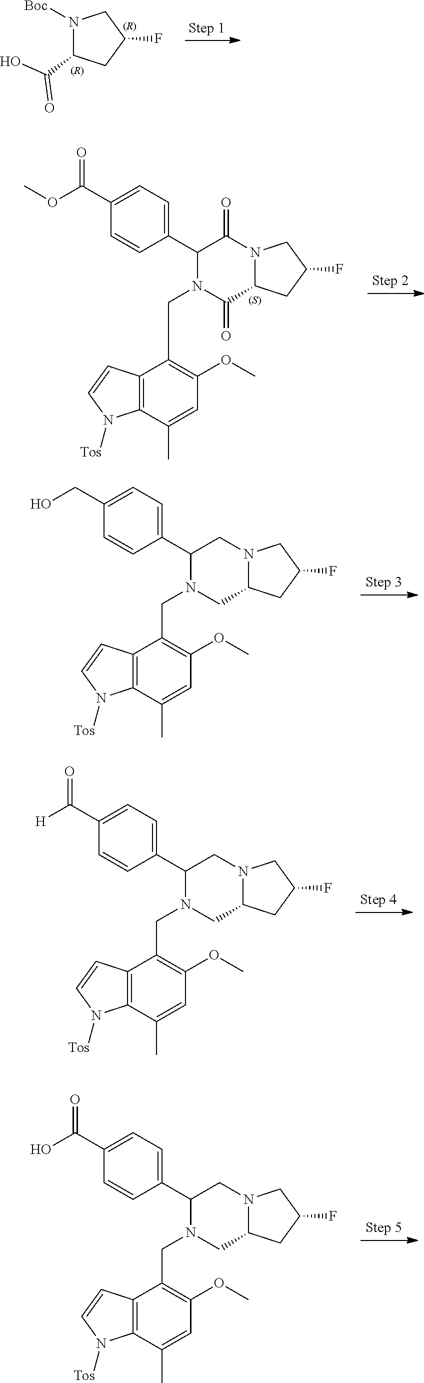

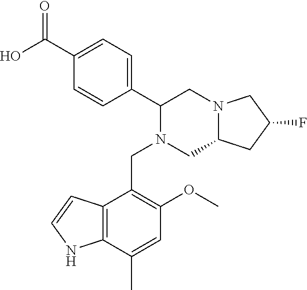

Step 1. Methyl 4-((7R,8aR)-7-fluoro-2-((5-methoxy-7-methyl-1-tosyl-1H-indol-4-yl)methyl)-1,4-dioxooctahydropyrrolo[1,2-a]pyrazin-3-yl)benzoate was prepared following General Experimental Procedure 9. Methyl 4-formylbenzoate (286 mg, 1.74 mmol), (5-methoxy-7-methyl-1-tosyl-1H-indol-4-yl)methanamine (600 mg, 1.74 mmol), (2R,4R)-1-(tert-butoxycarbonyl)-4-fluoropyrrolidine-2-carboxylic acid (406 mg, 1.74 mmol), and 2-(2,2-dimethoxyethyl)-N-methylidynebenzenaminium (300 μL, 1.74 mmol) gave methyl 4-((7R,8aR)-7-fluoro-2-((5-methoxy-7-methyl-1-tosyl-1H-indol-4-yl)methyl)-1,4-dioxooctahydropyrrolo[1,2-a]pyrazin-3-yl)benzoate (155 mg, 14%) as a yellow oil. LCMS (General 3 Basic) RT: 1.75 min and 1.81 min; area % combined (254 nm): 95%; m/z=634.2 [M+H]⁺.

Step 2. (4-((7R,8aR)-7-Fluoro-2-((5-methoxy-7-methyl-1-tosyl-1H-indol-4-yl)methyl)octahydropyrrolo[1,2-a]pyrazin-3-yl)phenyl) methanol was prepared following General Experimental Procedure 10. Methyl 4-((7R,8aR)-7-fluoro-2-((5-methoxy-7-methyl-1-tosyl-1H-indol-4-yl)methyl)-1,4-dioxooctahydropyrrolo[1,2-a]pyrazin-3-yl)benzoate gave crude (4-((7R,8aR)-7-fluoro-2-((5-methoxy-7-methyl-1-tosyl-1H-indol-4-yl)methyl)octahydropyrrolo[1,2-a]pyrazin-3-yl)phenyl) methanol (150 mg) as a brown oil. LCMS (General 3 Basic) RT: 1.85 min; area % (254 nm): 27%; m/z=578.4 [M+H]⁺.

Step 3. 4-((7R,8aR)-7-Fluoro-2-((5-methoxy-7-methyl-1-tosyl-1H-indol-4-yl)methyl)octahydropyrrolo[1,2-a]pyrazin-3-yl)benzaldehyde was prepared according to the following procedure. To a solution of (4-((7R,8aR)-7-fluoro-2-((5-methoxy-7-methyl-1-tosyl-1H-indol-4-yl)methyl)octahydropyrrolo[1,2-a]pyrazin-3-yl)phenyl) methanol (150 mg, 1.0 eq, 260 μmol) in DCM (6 mL) was added manganese(IV) oxide (226 mg, 10 eq, 2.60 mmol) and the reaction mixture stirred at 20° C. for 15 h. LCMS indicated incomplete conversion, manganese(IV) oxide (226 mg, 10 Eq, 2.60 mmol) was added and the mixture stirred at 20° C. for 15 h. The reaction was worked-up; filtered over Celite®, washed the filtrate with sat. aq. NaHCO₃, dried over Na₂SO₄, filtered, and concentrated under reduced pressure to afford crude 4-((7R,8aR)-7-fluoro-2-((5-methoxy-7-methyl-1-tosyl-1H-indol-4-yl)methyl)octahydropyrrolo[1,2-a]pyrazin-3-yl)benzaldehyde (145 mg) as a brown oil. LCMS (General 3 Basic) RT: 1.85 min; area % (254 nm): 31%; m/z=576.5 [M+H]⁺.

Step 4. 4-((7R,8aR)-7-Fluoro-2-((5-methoxy-7-methyl-1-tosyl-1H-indol-4-yl)methyl)octahydropyrrolo[1,2-a]pyrazin-3-yl)benzoic acid was prepared according to the following procedure. To a solution of 4-((7R,8aR)-7-fluoro-2-((5-methoxy-7-methyl-1-tosyl-1H-indol-4-yl)methyl)octahydropyrrolo[1,2-a]pyrazin-3-yl)benzaldehyde (145 mg, 1.0 eq, 252 μmol) in tert-BuOH (2.0 mL), CH₃CN (0.50 mL) and H₂O (0.50 mL) was added 2-methyl-2-butene (2.09 mL, 80 eq, 20.1 mmol). Then, a solution of sodium chlorite (42.7 mg, 80% Wt, 1.5 eq, 378 μmol), NaH₂PO₄ (243 mg, 7.0 eq, 1.76 mmol) in H₂O (0.75 mL) was added dropwise. The resulting mixture was stirred at 20° C. for 15 h. LCMS indicated full conversion. The reaction was worked-up; concentrated under reduced pressure and purified the residue over an Isolute® SCX-2 catch and release column to afford crude 4-((7R,8aR)-7-fluoro-2-((5-methoxy-7-methyl-1-tosyl-1H-indol-4-yl)methyl)octahydropyrrolo[1,2-a]pyrazin-3-yl)benzoic acid (150 mg) as a brown oil. LCMS (General 3 Basic) RT: 1.32 min; area % (254 nm): 54%; m/z=590.3 [M−H]⁻.

Step 5. 4-((7R,8aR)-7-Fluoro-2-((5-methoxy-7-methyl-1H-indol-4-yl)methyl)octahydropyrrolo[1,2-a]pyrazin-3-yl)benzoic acid was prepared according to the following procedure. To a solution of 4-((7R,8aR)-7-fluoro-2-((5-methoxy-7-methyl-1-tosyl-1H-indol-4-yl)methyl)octahydropyrrolo[1,2-a]pyrazin-3-yl)benzoic acid (150 mg, 1.0 eq, 254 μmol) in dry THF (2.0 mL) in a pressure tube was added 1.0 M TBAF in THF (1.52 mL, 6.0 eq, 1.52 mmol) and the resulting mixture was heated at 90° C. for 4 h. The reaction was worked-up, concentrated under reduced pressure and purified the residue by automated reverse phase FCC to afford the desired product with a TBAF residue. This material was submitted for preparative HPLC to afford 4-((7R,8aR)-7-fluoro-2-((5-methoxy-7-methyl-1H-indol-4-yl)methyl)octahydropyrrolo[1,2-a]pyrazin-3-yl)benzoic acid (4.0 mg, 3.6% over 4 steps) as a white solid. LCMS (31697 LCMS-5 C3) RT: 1.697 min; area % (215 nm): 85.7%; m/z=438.2 [M+H]⁺. ¹H-NMR (400 MHz, CD₃OD) δ 8.08 (d, J=8.0 Hz, 2H), 7.66 (d, J=7.5 Hz, 2H), 7.23 (d, J=3.2 Hz, 1H), 6.70 (s, 1H), 6.40 (d, J=3.2 Hz, 1H), 5.37-5.01 (m, 1H), 4.58 (s, 2H), 4.05-3.84 (m, 2H), 3.76 (s, 3H), 3.70-3.53 (m, 1H), 3.29-3.16 (m, 3H), 3.16-3.00 (m, 2H), 2.47 (s, 4H), 2.44-2.37 (m, 1H), 2.38-2.15 (m, 3H). 19F-NMR (CD₃OD) δ −76.94.

### Example 94. 4-((6R,8aS)-2-((5-Methoxy-7-methyl-1H-indol-4-yl)methyl)-6-methyloctahydropyrrolo[1,2-a]pyrazin-3-yl)benzoic acid

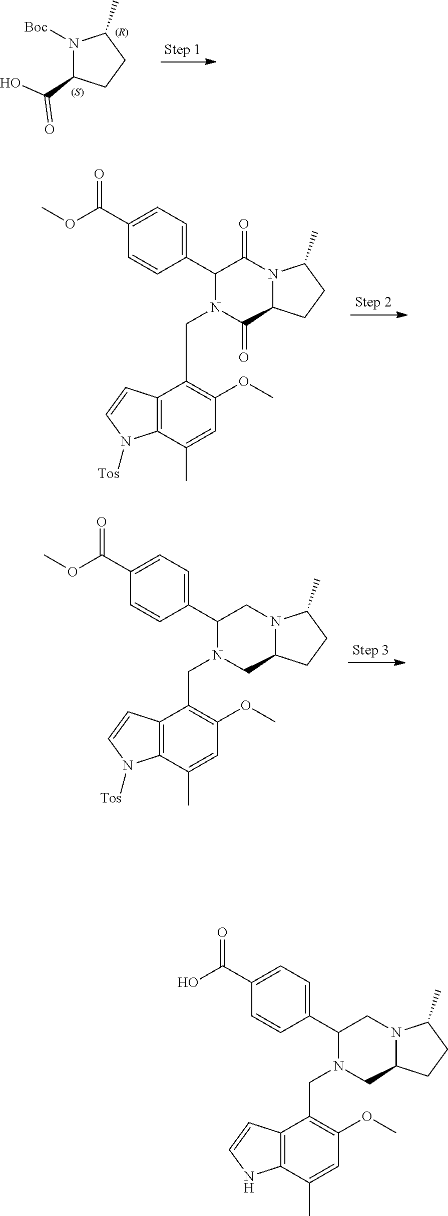

Step 1. Methyl 4-((6R,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate was prepared following General Experimental Procedure 9. Methyl 4-formylbenzoate (334 mg, 1.0 eq, 2.03 mmol), (5-methoxy-7-methyl-1-tosyl-1H-indol-4-yl) methanamine (700 mg, 1.0 eq, 2.03 mmol), and (2S,5R)-1-(tert-butoxycarbonyl)-5-methylpyrrolidine-2-carboxylic acid (466 mg, 1.0 eq, 2.03 mmol) gave methyl 4-((6R,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate (493 mg, 39%) as a dark green solid. LCMS (General 3 Basic) RT: 1.77 and 1.80 min; area % combined (254 nm): 90%; m/z=630.4 [M+H]⁺.

Step 2. Methyl 4-((6R,8aS)-2-((5-methoxy-7-methyl-1-tosyl-1H-indol-4-yl)methyl)-6-methyloctahydropyrrolo[1,2-a]pyrazin-3-yl)benzoate was prepared following General Experimental Procedure 10. Methyl 4-((6R,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate (480 mg, 762 μmol) gave crude methyl 4-((6R,8aS)-2-((5-methoxy-7-methyl-1-tosyl-1H-indol-4-yl)methyl)-6-methyloctahydropyrrolo[1,2-a]pyrazin-3-yl)benzoate (440 mg). LCMS (General 3 Basic) RT: 2.16 min; area % (254 nm): 56%; m/z=602.4 [M+H]⁺.

Step 3. 4-((6R,8aS)-2-((5-Methoxy-7-methyl-1H-indol-4-yl)methyl)-6-methyloctahydropyrrolo[1,2-a]pyrazin-3-yl)benzoic acid was prepared following General Experimental Procedure 11. Crude methyl 4-((6R,8aS)-2-((5-methoxy-7-methyl-1-tosyl-1H-indol-4-yl)methyl)-6-methyloctahydropyrrolo[1,2-a]pyrazin-3-yl)benzoate (440 mg) gave 4-((6R,8aS)-2-((5-methoxy-7-methyl-1H-indol-4-yl)methyl)-6-methyloctahydropyrrolo[1,2-a]pyrazin-3-yl)benzoic acid (7.1 mg, 2.2%) as a white solid. LCMS (31697 LCMS-5 C3) RT: 1.554 and 1.672 min; area % combined (215 nm): >80%; m/z=434.2 [M+H]⁺. ¹H-NMR (400 MHz, CD₃OD) δ 8.12-7.92 (m, 2H), 7.60 (dd, J=8.2, 2.9 Hz, 2H), 7.18 (t, J=3.1 Hz, 1H), 6.68 (d, J=2.9 Hz, 1H), 6.48 (dd, J=8.6, 3.1 Hz, 1H), 3.91-3.66 (m, 4H), 3.67-3.36 (m, 3H), 3.31 (dt, J=3.3, 1.6 Hz, 6H), 3.25-3.04 (m, 1H), 2.99-2.80 (m, 2H), 2.71 (dd, J=11.9, 3.6 Hz, 1H), 2.64 (dd, J=13.2, 3.8 Hz, OH), 2.46 (d, J=0.9 Hz, 3H), 2.25 (td, J=16.2, 8.7 Hz, 1H), 2.18-2.02 (m, 2H), 1.98-1.76 (m, 1H), 1.76-1.57 (m, 1H), 1.53-1.21 (m, 2H), 1.16 (d, J=6.7 Hz, 1H), 1.10 (d, J=6.1 Hz, OH), 1.05 (d, J=6.3 Hz, 2H).

### Example 95. Diastereomer 1 of 4-((6R,8aS)-2-((5-methoxy-7-methyl-1H-indol-4-yl)methyl)-6-methyloctahydropyrrolo[1,2-a]pyrazin-3-yl)benzoic acid

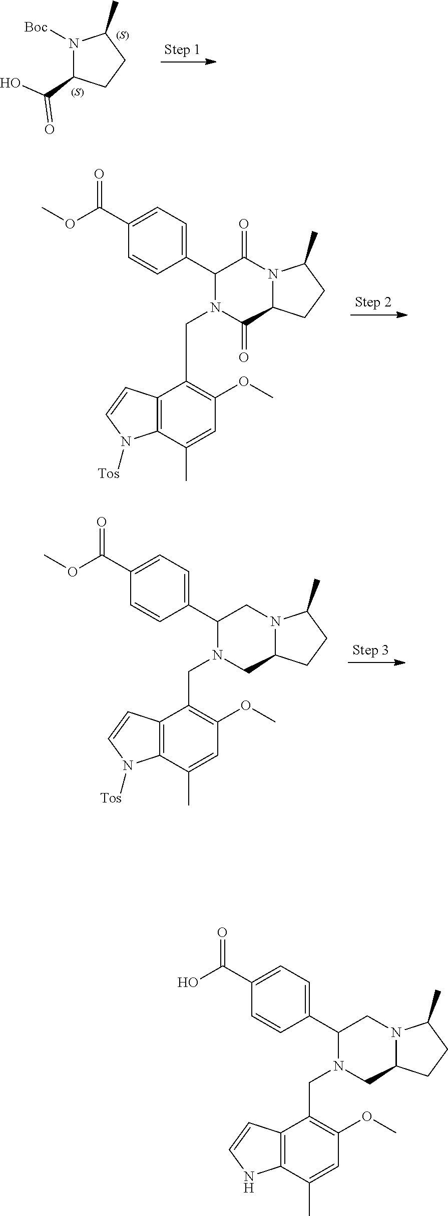

Step 1. Diastereomer 1 of methyl 4-((6S,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate was prepared following General Experimental Procedure 9. Methyl 4-formylbenzoate (310 mg, 1.0 eq, 1.89 mmol), (5-methoxy-7-methyl-1-tosyl-1H-indol-4-yl) methanamine (650 mg, 1.0 eq, 1.89 mmol), and (2S,5R)-1-(tert-butoxycarbonyl)-5-methylpyrrolidine-2-carboxylic acid (433 mg, 1.0 eq, 1.89 mmol) gave diastereomer 1 of methyl 4-((6S,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate (58 mg, 4.9%) as a brown oil. First eluting diastereomer (General 3 Basic) RT: 1.74 min; area % (254 nm): 93%; m/z=630.2 [M+H]⁺.

Step 2. Diastereomer 1 of methyl 4-((6S,8aS)-2-((5-methoxy-7-methyl-1-tosyl-1H-indol-4-yl)methyl)-6-methyloctahydropyrrolo[1,2-a]pyrazin-3-yl)benzoate was prepared following General Experimental Procedure 10. Diastereomer 1 of methyl 4-((6S,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate (58 mg, 122 μmol) gave crude diastereomer 1 of methyl 4-((6S,8aS)-2-((5-methoxy-7-methyl-1-tosyl-1H-indol-4-yl)methyl)-6-methyloctahydropyrrolo[1,2-a]pyrazin-3-yl)benzoate (45 mg). LCMS (General 3 Basic) RT: 2.46 min; area % (254 nm): 63%; m/z=602.5 [M+H]⁺.

Step 3. Diastereomer 1 of 4-((6S,8aS)-2-((5-methoxy-7-methyl-1H-indol-4-yl)methyl)-6-methyloctahydropyrrolo[1,2-a]pyrazin-3-yl)benzoic acid was prepared following General Experimental Procedure 11. Crude diastereomer 1 of methyl 4-((6R,8aS)-2-((5-methoxy-7-methyl-1-tosyl-1H-indol-4-yl)methyl)-6-methyloctahydropyrrolo[1,2-a]pyrazin-3-yl)benzoate (45 mg) gave diastereomer 1 of 4-((6R,8aS)-2-((5-methoxy-7-methyl-1H-indol-4-yl)methyl)-6-methyloctahydropyrrolo[1,2-a]pyrazin-3-yl)benzoic acid (2.0 mg, 6%) as a white solid. LCMS (31697 LCMS-5 C3) RT: 1.595 min; area % (215 nm): 78.6%; m/z=434.2 [M+H]⁺.

### Example 96. Diastereomer 2 of 4-((6R,8aS)-2-((5-methoxy-7-methyl-1H-indol-4-yl)methyl)-6-methyloctahydropyrrolo[1,2-a]pyrazin-3-yl)benzoic acid

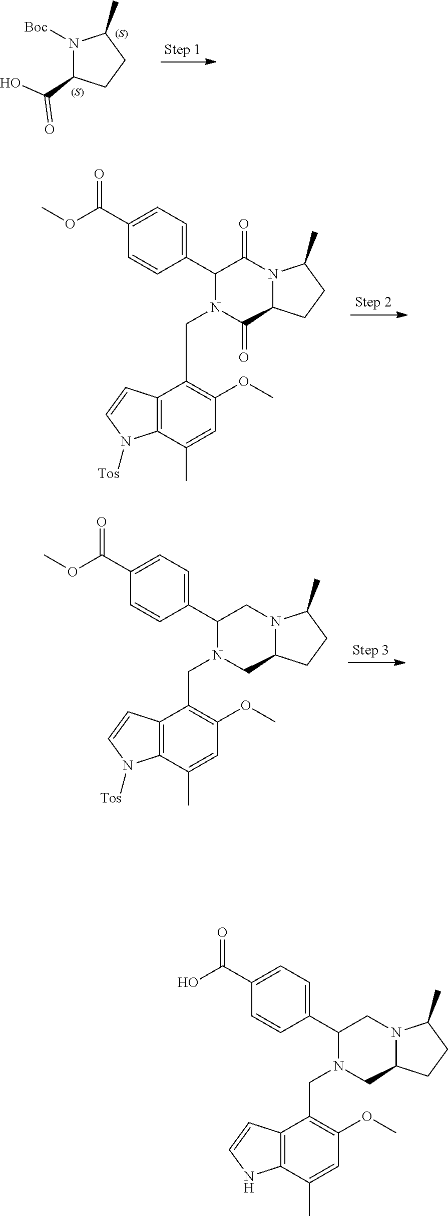

Step 1. Diastereomer 2 of methyl 4-((6S,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate was prepared following General Experimental Procedure 9. Methyl 4-formylbenzoate (310 mg, 1.0 eq, 1.89 mmol), (5-methoxy-7-methyl-1-tosyl-1H-indol-4-yl) methanamine (650 mg, 1.0 eq, 1.89 mmol), and (2S,5R)-1-(tert-butoxycarbonyl)-5-methylpyrrolidine-2-carboxylic acid (433 mg, 1.0 eq, 1.89 mmol) gave diastereomer 2 of methyl 4-((6S,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate (182 mg, 15%) as a brown oil. Second eluting diastereomer (General 3 Basic) RT: 1.81 min; area % (254 nm): 100%; m/z=630.2 [M+H]⁺.

Step 2. Diastereomer 2 of methyl 4-((6S,8aS)-2-((5-methoxy-7-methyl-1-tosyl-1H-indol-4-yl)methyl)-6-methyloctahydropyrrolo[1,2-a]pyrazin-3-yl)benzoate was prepared following General Experimental Procedure 10. Diastereomer 2 of methyl 4-((6S,8aS)-2-((5-methoxy-7-methyl-1-tosyl-1H-indol-4-yl)methyl)-6-methyl-1,4-dioxooctahydropyrrolo[1,2-a]pyrazin-3-yl)benzoate (182 mg, 289 μmol) gave crude diastereomer 2 of methyl 4-((6S,8aS)-2-((5-methoxy-7-methyl-1-tosyl-1H-indol-4-yl)methyl)-6-methyloctahydropyrrolo[1,2-a]pyrazin-3-yl)benzoate (160 mg). LCMS (General 3 Basic) RT: 2.24 min; area % (254 nm): 66%; m/z=602.6 [M+H]⁺.

Step 3. Diastereomer 2 of 4-((6S,8aS)-2-((5-methoxy-7-methyl-1H-indol-4-yl)methyl)-6-methyloctahydropyrrolo[1,2-a]pyrazin-3-yl)benzoic acid was prepared following General Experimental Procedure 11. Crude diastereomer 2 of methyl 4-((6R,8aS)-2-((5-methoxy-7-methyl-1-tosyl-1H-indol-4-yl)methyl)-6-methyloctahydropyrrolo[1,2-a]pyrazin-3-yl)benzoate (160 mg) gave diastereomer 2 of 4-((6R,8aS)-2-((5-methoxy-7-methyl-1H-indol-4-yl)methyl)-6-methyloctahydropyrrolo[1,2-a]pyrazin-3-yl)benzoic acid (10 mg, 9%) as a white solid. LCMS (31697 LCMS-5 C3) RT: 1.745 min; area % (215 nm): 96.8%; m/z=434.2 [M+H]⁺. ¹H-NMR (400 MHz, CD₃OD) δ 8.26-8.06 (m, 2H), 7.76 (d, J=7.8 Hz, 2H), 7.26 (d, J=3.1 Hz, 1H), 6.73 (s, 1H), 6.37 (d, J=3.1 Hz, 1H), 4.04 (s, 2H), 3.76 (s, 4H), 3.63-3.50 (m, 1H), 3.03 (s, 2H), 2.88 (s, 1H), 2.65 (d, J=7.5 Hz, 1H), 2.48 (d, J=0.8 Hz, 3H), 2.20 (s, 1H), 1.97 (s, 1H), 1.59 (d, J=9.0 Hz, 2H), 1.30 (d, J=6.3 Hz, 3H).

### Example 97. Diastereomer 2 of 4-((9aR)-8,8-difluoro-2-((5-methoxy-7-methyl-1H-indol-4-yl)methyl)octahydro-2H-pyrido[1,2-a]pyrazin-3-yl)benzoic acid

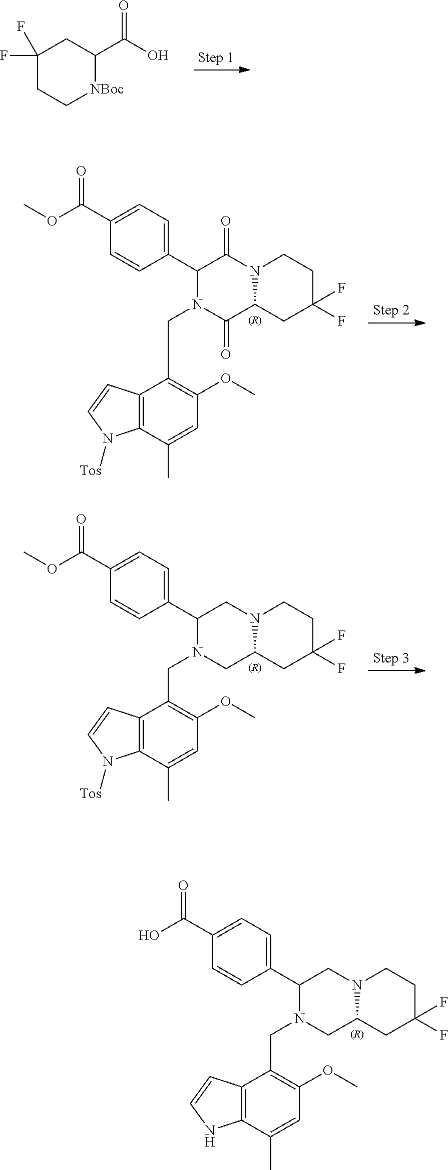

Step 1. Methyl 4-((9aR)-8,8-difluoro-2-((5-methoxy-7-methyl-1-tosyl-1H-indol-4-yl)methyl)-1,4-dioxooctahydro-2H-pyrido[1,2-a]pyrazin-3-yl)benzoate was prepared following General Experimental Procedure 9. Methyl 4-formylbenzoate (286 mg, 1.74 mmol), (5-methoxy-7-methyl-1-tosyl-1H-indol-4-yl) methanamine (600 mg, 1.74 mmol), 1-(tert-butoxycarbonyl)-4,4-difluoropiperidine-2-carboxylic acid (462 mg, 1.74 mmol), and 2-(2,2-dimethoxyethyl)-N-methylidynebenzenaminium (300 μL, 1.74 mmol) gave diastereomer 2 of methyl 4-((9aR)-8,8-difluoro-2-((5-methoxy-7-methyl-1-tosyl-1H-indol-4-yl)methyl)-1,4-dioxooctahydro-2H-pyrido[1,2-a]pyrazin-3-yl)benzoate (220 mg, 22%) as a yellow oil. LCMS (General 3 Basic) RT: 1.85 min; area % (254 nm): 93%; m/z=666.4 [M+H]⁺.

Step 2. Methyl 4-((9aR)-8,8-difluoro-2-((5-methoxy-7-methyl-1-tosyl-1H-indol-4-yl)methyl)octahydro-2H-pyrido[1,2-a]pyrazin-3-yl)benzoate was prepared following General Experimental Procedure 10. Methyl 4-((9aR)-8,8-difluoro-2-((5-methoxy-7-methyl-1-tosyl-1H-indol-4-yl)methyl)-1,4-dioxooctahydro-2H-pyrido[1,2-a]pyrazin-3-yl)benzoate (120 mg, 180 μmol) gave methyl 4-((9aR)-8,8-difluoro-2-((5-methoxy-7-methyl-1-tosyl-1H-indol-4-yl)methyl)octahydro-2H-pyrido[1,2-a]pyrazin-3-yl)benzoate (120 mg, 104%). LCMS (General 3 Basic) RT: 2.10 min and 2.15; area % combined (254 nm): 29%; m/z=638.4 [M+H]⁺.

Step 3. Diastereomer 2 of 4-((9aR)-8,8-difluoro-2-((5-methoxy-7-methyl-1H-indol-4-yl)methyl)octahydro-2H-pyrido[1,2-a]pyrazin-3-yl)benzoic acid was prepared following General Experimental Procedure 11. Methyl 4-((9aR)-8,8-difluoro-2-((5-methoxy-7-methyl-1-tosyl-1H-indol-4-yl)methyl)octahydro-2H-pyrido[1,2-a]pyrazin-3-yl)benzoate (112 mg, 176 μmol) gave diastereomer 2 of 4-((9aR)-8,8-difluoro-2-((5-methoxy-7-methyl-1H-indol-4-yl)methyl)octahydro-2H-pyrido[1,2-a]pyrazin-3-yl)benzoic acid (5.29 mg, 5%) as a white solid. LCMS (31697 LCMS-5 C3) RT: 2.140 min; area % (215 nm): 97.9%; m/z=470.2 [M+H]⁺. ¹H NMR (400 MHz, CD₃OD) δ 7.99 (d, 2H), 7.74 (d, J=7.9 Hz, 2H), 7.23 (d, J=3.1 Hz, 1H), 6.70 (s, 1H), 6.55 (d, J=3.1 Hz, 1H), 3.80 (s, 4H), 3.69-3.47 (m, 2H), 3.18-3.09 (m, 1H), 2.96 (d, J=13.1 Hz, 1H), 2.80 (s, 1H), 2.75-2.63 (m, 3H), 2.56 (d, J=12.1 Hz, 1H), 2.48 (d, J=0.8 Hz, 3H), 2.28-2.02 (m, 3H), 1.91-1.79 (m, 1H), 1.65-1.53 (m, 1H). ¹⁹F NMR (376 MHz, CD₃OD) δ −76.94.

### Example 98. 4-((9aS)-3-((5-methoxy-7-methyl-1H-indol-4-yl)methyl)octahydro-1H-pyrrolo[1,2-d][1,4]diazepin-4-yl)benzoic acid

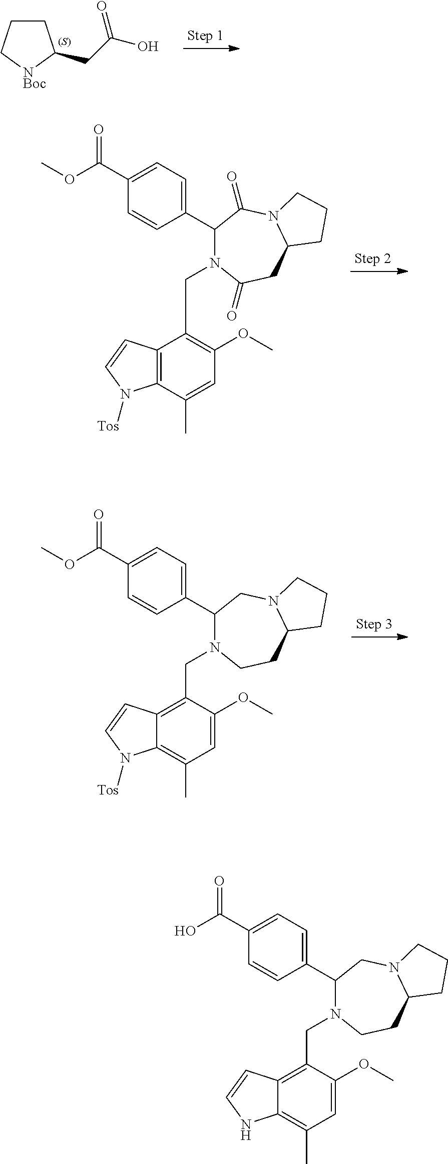

Step 1. Methyl 4-((9aS)-3-((5-methoxy-7-methyl-1-tosyl-1H-indol-4-yl)methyl)-2,5-dioxooctahydro-1H-pyrrolo[1,2-d][1,4]diazepin-4-yl)benzoate was prepared following General Experimental Procedure 9, with the exception that the resulting residue was dissolved in toluene, Et₃N (50 eq.) added and the mixture heated at 100° C. for 16 h. Methyl 4-formylbenzoate (158 mg, 0.96 mmol), (5-methoxy-7-methyl-1-tosyl-1H-indol-4-yl) methanamine (330 mg, 0.96 mmol), 2-(2,2-dimehtoxyethyl)-N-methylynebenzaminium (0.17 mL, 0.96 mmol), and(S)-2-(1-(tert-butoxycarbonyl) pyrrolidin-2-yl) acetic acid (220 mg, 0.96 mmol) gave methyl 4-((9aS)-3-((5-methoxy-7-methyl-1-tosyl-1H-indol-4-yl)methyl)-2,5-dioxooctahydro-1H-pyrrolo[1,2-d][1,4]diazepin-4-yl)benzoate (70 mg, 12%). LCMS (General 3 Basic) RT: 1.78 min; area % (254 nm): 100%; m/z=630.3 [M+H]⁺.

Step 2. Methyl 4-((9aS)-3-((5-methoxy-7-methyl-1-tosyl-1H-indol-4-yl)methyl)octahydro-1H-pyrrolo[1,2-d][1,4]diazepin-4-yl)benzoate was prepared following General Experimental Procedure 10. 4-((9aS)-3-((5-Methoxy-7-methyl-1-tosyl-1H-indol-4-yl)methyl)-2,5-dioxooctahydro-1H-pyrrolo[1,2-d][1,4]diazepin-4-yl)benzoate (70 mg, 0.11 mmol) gave methyl 4-((9aS)-3-((5-methoxy-7-methyl-1-tosyl-1H-indol-4-yl)methyl)octahydro-1H-pyrrolo[1,2-d][1,4]diazepin-4-yl)benzoate (80 mg, 42%). LCMS (General 3 Basic) RT: 2.46 min; area % (254 nm): 35%; m/z=602.4 [M+H]⁺.

Step 3. 4-((9aS)-3-((5-Methoxy-7-methyl-1H-indol-4-yl)methyl)octahydro-1H-pyrrolo[1,2-d][1,4]diazepin-4-yl)benzoic acid was prepared following General Experimental Procedure 11, with the exception that lithium hydroxide monohydrate (11 mg, 2.0 eq, 0.27 mmol) was used instead of 1.0 M aq. NaOH. Methyl 4-((9aS)-3-((5-methoxy-7-methyl-1-tosyl-1H-indol-4-yl)methyl)octahydro-1H-pyrrolo[1,2-d][1,4]diazepin-4-yl)benzoate (80 mg) gave 4-((9aS)-3-((5-methoxy-7-methyl-1H-indol-4-yl)methyl)octahydro-1H-pyrrolo[1,2-d][1,4]diazepin-4-yl)benzoic acid (1.36 mg, 2.3%). LCMS-5 C3) RT: 1.576 min; area % (215 nm): 96%; m/z=434.2 [M+H]⁺. ¹H-NMR (400 MHz, CD₃OD) δ 8.07 (d, J=8.2 Hz, 2H), 7.63 (s, 2H), 7.26 (s, 1H), 6.73 (s, 1H), 6.38 (d, J=3.2 Hz, 1H), 4.57 (s, 2H), 4.38-3.93 (m, 2H), 3.79-3.71 (m, 4H), 3.41-3.33 (m, 3H), 3.18-3.08 (m, 2H), 2.48 (s, 3H), 2.24-2.11 (m, 2H), 2.06-1.95 (m, 2H), 1.92-1.80 (m, 1H), 1.73-1.57 (m, 1H).

### Example 99. 4-(4-((5-Methoxy-7-methyl-1H-indol-4-yl)methyl)-1-methyl-1,4-diazepan-5-yl)benzoic acid

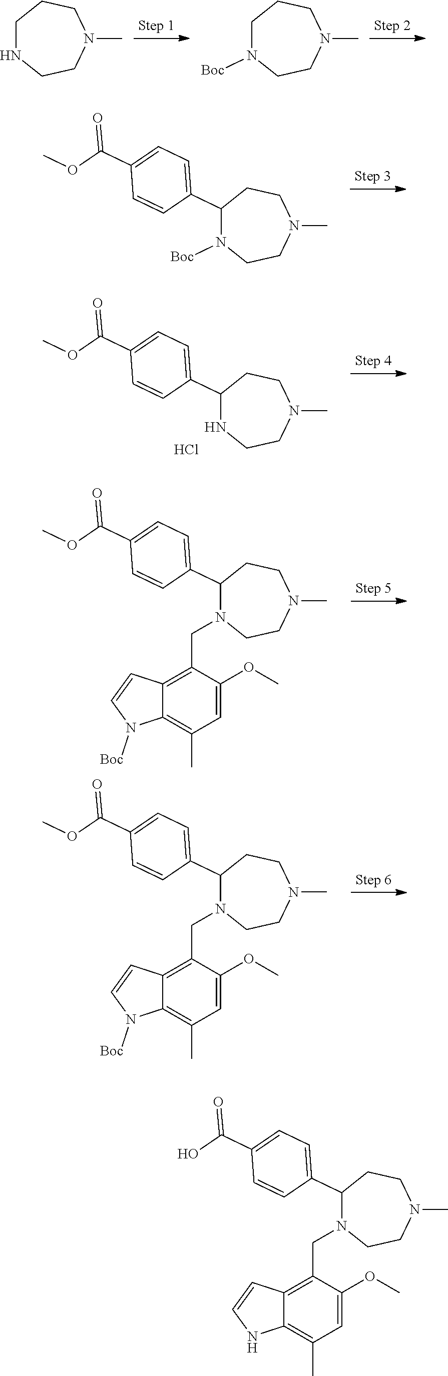

Step 1. tert-Butyl 4-methyl-1,4-diazepane-1-carboxylate was prepared with the following procedure. To a solution of 1-methyl-1,4-diazepane (3.62 mL, 1.0 eq, 29.1 mmol) in DCM (7.3 mL), Boc₂O (7.62 g, 1.2 eq, 34.9 mmol) was added at once. The reaction was stirred at 20° C. for 15 h. The reaction was worked up; quenched with sat. aq. NaHCO₃ (10 mL) and the layers were allowed to separate. The aqueous layer was extracted with DCM. The combined organic layers were dried over Na₂SO₄, filtered, and concentrated under reduced pressure to afford crude tert-butyl 4-methyl-1,4-diazepane-1-carboxylate (5.72 g, 92%) as yellow oil. 1H-NMR (400 MHz, CDCl₃) δ 3.59-3.26 (m, 4H), 2.60-2.42 (m, 4H), 2.32 (s, 3H), 1.81 (dp, J=11.7, 6.1 Hz, 2H), 1.42 (s, 9H).

Step 2. tert-Butyl 2-(4-(methoxycarbonyl)phenyl)-4-methyl-1,4-diazepane-1-carboxylate was prepared with the following procedure. To a flame-dried, 100 mL 3-necked round bottom flask, under nitrogen, a solution of tert-butyl 4-methyl-1,4-diazepane-1-carboxylate (2.00 g, 1.54 eq, 9.31 mmol) and TMEDA (1.65 mL, 1.82 eq, 11.0 mmol) in anhydrous Et₂O (20 mL) was cooled to −78° C. and sec-butyllithium in cyclohexane/hexane (8.46 mL, 1.3 molar, 1.82 eq, 11.0 mmol) was slowly added via syringe. The reaction mixture was allowed to stir for 2 hrs at this temperature before a solution of zinc chloride in THF (15.7 mL, 0.7 molar, 1.82 eq, 11.0 mmol) was added. The reaction mixture was allowed to warm to room temperature and stirred at this temperature for 1 hour. In the meantime, a solution of Pd(PPh₃)₄ (335 mg, 0.0480 eq, 290 μmol) and 2-dicyclohexylphosphino-2,6-dimethoxy-1,1-biphenyl (266 mg, 0.107 eq, 647 μmol) was prepared in anhydrous toluene (20 mL) and allowed to stir for 15 min at room temperature. The catalyst solution was added to the piperidinyl zinc reagent followed by methyl 4-bromobenzoate (1.30 g, 1 eq, 6.05 mmol). This mixture was heated to 60° C. and allowed to stir at this temperature for 15 h. The reaction was worked-up; quenched with sat. aq. NH₄Cl and the aqueous layer was extracted with DCM (3×50 mL). The combined organic layer was dried over Na₂SO₄, filtered, and concentrated under reduced pressure. The crude was purified by automated normal phase FCC to afford tert-butyl 2-(4-(methoxycarbonyl)phenyl)-4-methyl-1,4-diazepane-1-carboxylate (370 mg, 18%). LCMS (LCMS-4 General3 AB) RT: 1.89 min; area % (254 nm): 94.7%; m/z=349.4 [M+H]⁺.

Step 3. Methyl 4-(4-methyl-1,4-diazepan-2-yl)benzoate hydrochloride was prepared with the following procedure. To a 250 mL round bottom flask containing tert-butyl 2-(4-(methoxycarbonyl)phenyl)-4-methyl-1,4-diazepane-1-carboxylate (370 mg, 1 eq, 1.06 mmol) DCM (5 mL) and 4 N HCl in dioxane (1.33 mL, 5 eq, 5.31 mmol) were added. The resulting mixture was allowed to stir at room temperature for 3 hours. The volatiles were evacuated under reduced pressure to afford crude methyl 4-(4-methyl-1,4-diazepan-2-yl)benzoate hydrochloride (341 mg, 100%). LCMS (LCMS-4 General3 AB) RT: 0.577 min; area % (254 nm): 94.6%; m/z=249.6 [M+H]⁺.

Step 4. tert-Butyl 5-methoxy-4-((7-(4-(methoxycarbonyl)phenyl)-4-methyl-1,4-diazepan-1-yl)methyl)-7-methyl-1H-indole-1-carboxylate was prepared with the following procedure. To a 250 mL round bottom flask containing methyl 4-(1-methyl-1,4-diazepan-5-yl)benzoate hydrochloride (371 mg, 1 eq, 1.15 mmol), THF (20 mL) and Et₃N (354 μL, 2.2 eq, 2.54 mmol) were added. The mixture was allowed to stir at room temperature for 30 minutes before adding 5-methoxy-7-methyl-1H-indole-4-carbaldehyde (328 mg, 1.5 eq, 1.73 mmol) and acetic acid (99.2 μL, 1.5 eq, 1.73 mmol). The mixture was allowed to stir for 1 hour before adding sodium triacetoxyborohydride (979 mg, 4 eq, 4.62 mmol). The resulting suspension was allowed to stir at room temperature for 15 h. The volatiles were evacuated under reduced pressure and the residue was purified by automated normal phase FCC to afford tert-butyl 5-methoxy-4-((7-(4-(methoxycarbonyl)phenyl)-4-methyl-1,4-diazepan-1-yl)methyl)-7-methyl-1H-indole-1-carboxylate (395 mg, 66%). LCMS (LCMS-4 General3 AB) RT: 2.37 min; area % (254 nm): 90.6%; m/z=522.4 [M+H]⁺.

Step 5. Enantiomer 1 of tert-butyl 5-methoxy-4-((7-(4-(methoxycarbonyl)phenyl)-4-methyl-1,4-diazepan-1-yl)methyl)-7-methyl-1H-indole-1-carboxylate was prepared with the following procedure. Chiral SFC purification (SFC method 31697-1176-09) was performed on racemic tert-butyl 5-methoxy-4-((7-(4-(methoxycarbonyl)phenyl)-4-methyl-1,4-diazepan-1-yl)methyl)-7-methyl-1H-indole-1-carboxylate (100 mg, 0.19 mmol) to afford enantiomer 1 of tert-butyl 5-methoxy-4-((7-(4-(methoxycarbonyl)phenyl)-4-methyl-1,4-diazepan-1-yl)methyl)-7-methyl-1H-indole-1-carboxylate (32.2 mg, 32%). First eluting enantiomer (UPC² 6m Cel2 MA1) RT: 3.315 min; area % (236 nm): 96.9% ee, m/z=522.3 [M+H]⁺.

Step 6. Enantiomer 1 of 4-(4-((5-methoxy-7-methyl-1H-indol-4-yl)methyl)-1-methyl-1,4-diazepan-5-yl)benzoic acid was prepared following General Experimental Procedure 8. Enantiomer 1 of tert-butyl 5-methoxy-4-((7-(4-(methoxycarbonyl)phenyl)-4-methyl-1,4-diazepan-1-yl)methyl)-7-methyl-1H-indole-1-carboxylate (32.2 mg) gave enantiomer 1 of 4-(4-((5-methoxy-7-methyl-1H-indol-4-yl)methyl)-1-methyl-1,4-diazepan-5-yl)benzoic acid (13.6 mg, 54%) as a white solid. LCMS (31697 LCMS-5 C3) RT: 1.42 min; area % (215 nm): 99.5%; m/z=408.2 [M+H]⁺. ¹H-NMR (400 MHz, CD₃OD) δ 7.97 (s, 1H), 7.58-7.47 (m, 1H), 6.26-6.16 (m, 1H), 4.85 (d, J=4.4 Hz, 8H), 4.80 (s, 6H), 3.88 (q, J=8.1, 7.4 Hz, 1H), 3.74 (s, 1H), 3.35 (s, 1H), 3.30-3.23 (m, 5H), 3.08 (s, 1H), 2.67 (d, J=17.4 Hz, 1H), 2.49-2.40 (m, 1H), 2.21 (s, 1H).

### Example 100. Enantiomer 2 of 4-(4-((5-Methoxy-7-methyl-1H-indol-4-yl)methyl)-1-methyl-1,4-diazepan-5-yl)benzoic acid

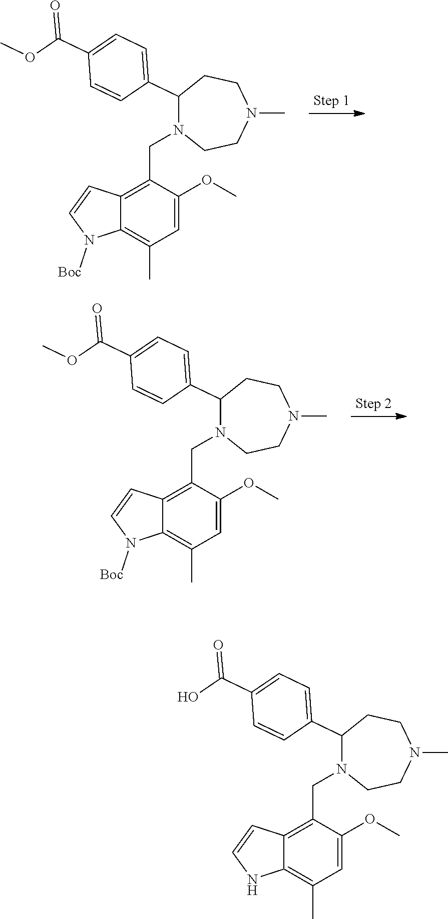

Step 1. Enantiomer 2 of tert-butyl 5-methoxy-4-((7-(4-(methoxycarbonyl)phenyl)-4-methyl-1,4-diazepan-1-yl)methyl)-7-methyl-1H-indole-1-carboxylate was prepared with the following procedure. Chiral SFC purification (SFC method 31697-1176-09) was performed on racemic tert-butyl 5-methoxy-4-((7-(4-(methoxycarbonyl)phenyl)-4-methyl-1,4-diazepan-1-yl)methyl)-7-methyl-1H-indole-1-carboxylate (100 mg, 0.19 mmol) to afford enantiomer 2 of tert-butyl 5-methoxy-4-((7-(4-(methoxycarbonyl)phenyl)-4-methyl-1,4-diazepan-1-yl)methyl)-7-methyl-1H-indole-1-carboxylate (20.0 mg, 20%). Second eluting enantiomer (UPC² 6m Cel2 MA1) RT: 3.315 min; area % (236 nm): 97.3% ee, m/z=522.3 [M+H]⁺.

Step 2. Enantiomer 2 of 4-(4-((5-methoxy-7-methyl-1H-indol-4-yl)methyl)-1-methyl-1,4-diazepan-5-yl)benzoic acid was prepared following General Experimental Procedure 8. Enantiomer 2 of tert-butyl 5-methoxy-4-((7-(4-(methoxycarbonyl)phenyl)-4-methyl-1,4-diazepan-1-yl)methyl)-7-methyl-1H-indole-1-carboxylate (20.0 mg) gave enantiomer 2 of 4-(4-((5-methoxy-7-methyl-1H-indol-4-yl)methyl)-1-methyl-1,4-diazepan-5-yl)benzoic acid (11.4 mg, 74%) as a white solid. LCMS (31697 LCMS-5 C3) RT: 1.42 min; area % (215 nm): 99.5%; m/z=408.2 [M+H]⁺. ¹H-NMR identical to reported above.

### Example 101. Enantiomer 1 of 4-(1-((5-methoxy-7-methyl-1H-indol-4-yl)methyl)-4-(2,2,2-trifluoroethyl)-1,4-diazepan-2-yl)benzoic acid

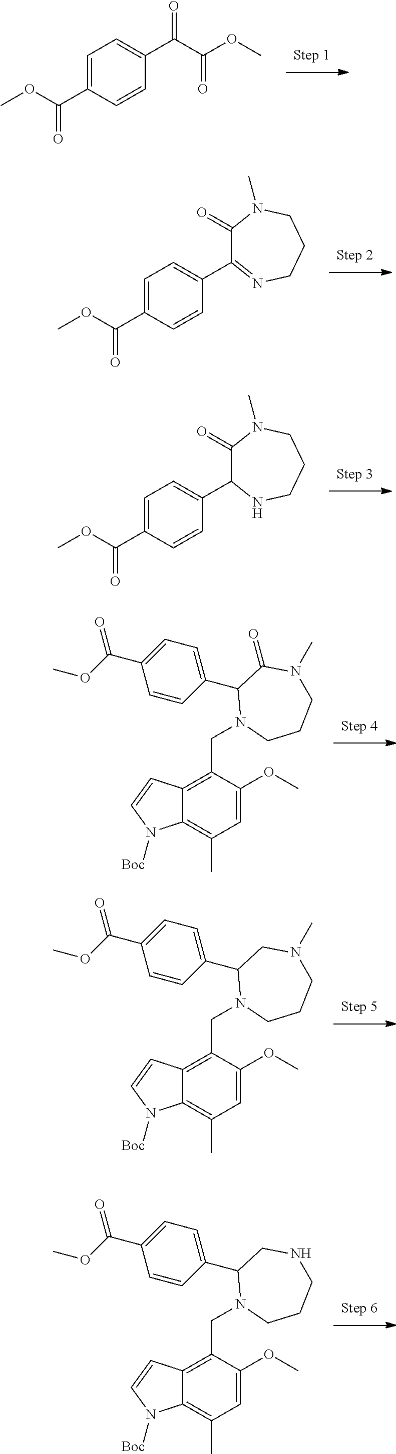

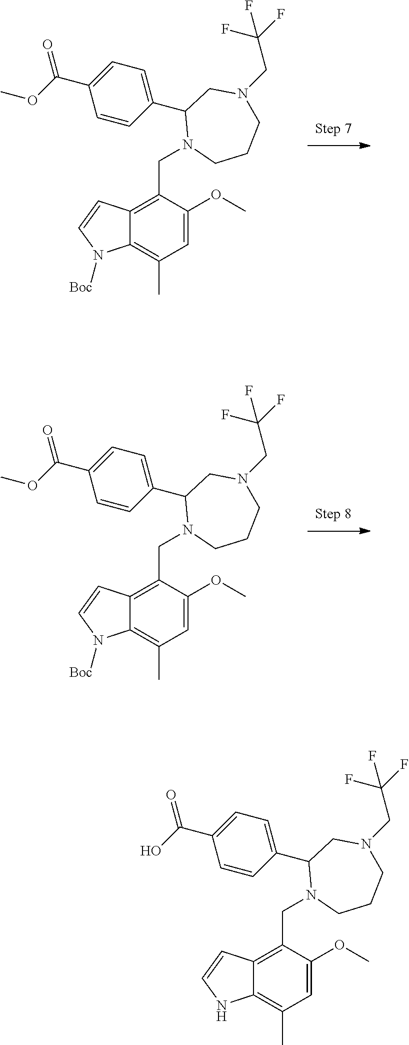 [SMILES: COC(=O)c1ccc(C2CN(CC(F)(F)F)CCCN2Cc2c(OC)cc(C)c3c2ccn3C(=O)OC(C)(C)C)cc1 ; COC(=O)c1ccc(C2CN(CC(F)(F)F)CCCN2Cc2c(OC)cc(C)c3c2ccn3C(=O)OC(C)(C)C)cc1 ; COc1cc(C)c2[nH]ccc2c1CN1CCCN(CC(F)(F)F)CC1c1ccc(C(=O)O)cc1]

Step 1. Methyl 4-(1-methyl-2-oxo-2,5,6,7-tetrahydro-1H-1,4-diazepin-3-yl)benzoate was prepared with the following procedure. To a round bottom flask were charged toluene (40 mL), N-methylpropane-1,3-diamine (1.52 g, 1.1 eq, 17.3 mmol), methyl 4-(2-methoxy-2-oxoacetyl)benzoate (3.49 g, 1.0 eq, 15.7 mmol) and acetic acid (989 μL, 1.1 eq, 17.3 mmol). The resulting mixture was stirred at reflux for 1 hour. The reaction was worked-up; cooled down to 20° C. and diluted with EtOAc (50 mL). The organic layer was washed with sat. aq. NaHCO₃ (2×25 mL), H₂O (2×25 mL), dried over Na₂SO₄, filtered, and concentrated under reduced pressure to afford crude methyl 4-(1-methyl-2-oxo-2,5,6,7-tetrahydro-1H-1,4-diazepin-3-yl)benzoate (3.9 g) as a yellow oil. LCMS (LCMS-4 General 3 AB) RT: 1.37 min; area % (254 nm): 67.4%; m/z=261.4 [M+H^{]+}.

Step 2. Methyl 4-(4-methyl-3-oxo-1,4-diazepan-2-yl)benzoate was prepared with the following procedure. Crude methyl 4-(1-methyl-2-oxo-2,5,6,7-tetrahydro-1H-1,4-diazepin-3-yl)benzoate (3.9 g) was dissolved in MeOH (40 mL). Then 10% Pd/C (1.6 g, 0.1 eq, 1.5 mmol) was added and the reaction mixture was stirred under H₂ atmosphere (balloon) at 20° C. for 15 h. The reaction was worked-up; filtered over Celite® and the filter cake washed with MeOH (2×10 mL). The filtrate was concentrated under reduced pressure to afford crude methyl 4-(4-methyl-3-oxo-1,4-diazepan-2-yl)benzoate (3.45 g) as an orange oil. LCMS (LCMS-4 General3 AB) RT: 0.85 min; area % (215 nm): 86.3%; m/z=263.4 [M+H]+

Step 3. tert-Butyl 5-methoxy-4-((2-(4-(methoxycarbonyl)phenyl)-4-methyl-3-oxo-1,4-diazepan-1-yl)methyl)-7-methyl-1H-indole-1-carboxylate was prepared with the following procedure. Methyl 4-(4-methyl-3-oxo-1,4-diazepan-2-yl)benzoate (3.09 g, 1.0 eq, 11.8 mmol), tert-butyl-4-formyl-5-methoxy-7-methyl-1H-indole-1-carboxylate (5.11 g, 1.5 eq, 17.7 mmol) and acetic acid (742 μL, 1.1 eq, 13.0 mmol) were dissolved in 1,2-dichloroethane (50 mL) and stirred at 20° C. for 1 hour. Then sodium triacetoxyborohydride (9.99 g, 4.0 eq, 47.1 mmol) was added and stirred at 20° C. for 72 h. The reaction was worked-up; mixture was diluted with DCM (50 mL) and washed with sat. aq. NaHCO₃ (2×20 mL). The organic layer was washed with H₂O (50 mL), brine, dried with Na₂SO₄, and concentrated under reduced pressure. The crude was purified on an Isolute® SCX-2 catch and release column (50 g) to afford tert-butyl 5-methoxy-4-((2-(4-(methoxycarbonyl)phenyl)-4-methyl-3-oxo-1,4-diazepan-1-yl)methyl)-7-methyl-1H-indole-1-carboxylate (4.03 g, 64%) as a light yellow foam. LCMS (LCMS-4 General2 AB) RT: 2.39 min; area % (215 nm): 78.9%; m/z=536.4 [M+H]⁺.

Step 4. tert-Butyl 5-methoxy-4-((2-(4-(methoxycarbonyl)phenyl)-4-methyl-1,4-diazepan-1-yl)methyl)-7-methyl-1H-indole-1-carboxylate was prepared with the following procedure. To a solution of tert-butyl 5-methoxy-4-((2-(4-(methoxycarbonyl)phenyl)-4-methyl-3-oxo-1,4-diazepan-1-yl)methyl)-7-methyl-1H-indole-1-carboxylate (2.0 g, 1.0 eq, 3.7 mmol) and acetylacetonato (1,5-cyclooctadiene) rhodium (58 mg, 0.05 eq, 0.19 mmol) under N₂ in THF (20 mL) at 0° C. was added silylbenzene (1.9 mL, 4.0 eq, 15 mmol). The ice bath was removed and temperature was gradually increased to 20° C. and subsequently at 60° C. for 15 h. The reaction was worked-up; concentrated under reduced pressure and the residue was purified by automated normal phase FCC to afford tert-butyl 5-methoxy-4-((2-(4-(methoxycarbonyl)phenyl)-4-methyl-1,4-diazepan-1-yl)methyl)-7-methyl-1H-indole-1-carboxylate (1.07 g, 55%) as light yellow foam. LCMS (LCMS-4 General2 AB) RT: 2.44 min; area % (215 nm): 92.3%; m/z=522.4 [M+H]⁺.

Step 5. tert-Butyl 5-methoxy-4-((2-(4-(methoxycarbonyl)phenyl)-1,4-diazepan-1-yl)methyl)-7-methyl-1H-indole-1-carboxylate was prepared with the following procedure. tert-Butyl 5-methoxy-4-((2-(4-(methoxycarbonyl)phenyl)-4-methyl-1,4-diazepan-1-yl)methyl)-7-methyl-1H-indole-1-carboxylate (1.00 g, 1.0 eq, 1.92 mmol) was dissolved in MeOH (10 mL). Then 35 wt. % aq. H₂O₂ (1.81 mL, 11.0 eq, 21.1 mmol) was added at 20° C. and subsequently heated at 60° C. for 2 hours. After cooling down, diluted with DCM (20 mL), washed with sat. aq. NaS₂O₄ (20 mL), H₂O, dried over Na₂SO₄, and concentrated under reduced pressure. The orange residue was dissolved in MeOH (15 mL) and FeSO₄·7H₂O (266 mg 0.5 eq, 958 μmol) was added. The resulting mixture was stirred at 20° C. for 2 hours. The reaction was worked-up; diluted with DCM (30 mL) and washed with H₂O (15 mL). The aqueous layer was extracted with DCM (3×10 mL). The combined organic layers were dried over Na₂SO₄ and concentrated under reduced pressure. The residue was purified by automated normal phase FCC to afford tert-butyl 5-methoxy-4-((2-(4-(methoxycarbonyl)phenyl)-1,4-diazepan-1-yl)methyl)-7-methyl-1H-indole-1-carboxylate (300 mg, 31%) as light yellow foam. LCMS (General 2 Basic) RT: 1.81 min; area % (254 nm): 73%; m/z=508.3 [M+H]⁺.

Step 6. tert-Butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate was prepared with the following procedure. tert-Butyl 5-methoxy-4-((2-(4-(methoxycarbonyl)phenyl)-1,4-diazepan-1-yl)methyl)-7-methyl-1H-indole-1-carboxylate (360 mg, 1.0 eq, 426 μmol), Et₃N (301 mg, 415 μL, 7.0 eq, 2.98 mmol) and 2,2,2-trifluoroethyl trifluoromethanesulfonate (307 μL, 5.0 eq, 2.13 mmol) were dissolved in THF (10 mL) and stirred at 60° C. for 15 h. The reaction was worked-up; cooled down and purified the residue by preparative HPLC to afford FCC to afford tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate (190 mg, 76%) obtained as brown oil. LCMS (General 2 Basic) RT: 2.41 min; area % (254 nm): 89%; m/z=590.3 [M+H]⁺.

Step 7. Enantiomer 1 of tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate was prepared according to the following procedure. Racemic tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate was separated by chiral SFC (100 mL 5% B) to afford enantiomer 1 of tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate (54 mg). First eluting enantiomer (31697M UPC² 10m IF MA1) RT: 5.610 min; area % (238 nm): >99% ee; m/z=590.3 [M+H]⁺.

Step 8. Enantiomer 1 of 4-(1-((5-methoxy-7-methyl-1H-indol-4-yl)methyl)-4-(2,2,2-trifluoroethyl)-1,4-diazepan-2-yl)benzoic acid was prepared following General Experimental Procedure 8. Enantiomer 1 of tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate (54 mg) gave enantiomer 1 of 4-(1-((5-methoxy-7-methyl-1H-indol-4-yl)methyl)-4-(2,2,2-trifluoroethyl)-1,4-diazepan-2-yl)benzoic acid (32.4 mg, 74%) as a white solid. LCMS (31697 LCMS-5 C3) RT: 1.961 min; area % (215 nm): >99%; m/z=476.2 [M+H]⁺. ¹H-NMR (400 MHz, CD₃OD) δ 8.09 (d, J=8.1 Hz, 2H), 7.55 (d, J=8.0 Hz, 2H), 7.28 (d, J=3.1 Hz, 1H), 6.71 (s, 1H), 6.21 (d, J=3.2 Hz, 1H), 4.52 (br s, 1H), 4.37 (s, 2H), 3.72-3.33 (m, 8H), 3.20-2.96 (m, 3H), 2.49 (s, 3H), 2.23-2.18 (m, 1H), 2.11-2.02 (m, 1H).

### Example 102. Enantiomer 2 of 4-(1-((5-methoxy-7-methyl-1H-indol-4-yl)methyl)-4-(2,2,2-trifluoroethyl)-1,4-diazepan-2-yl)benzoic acid

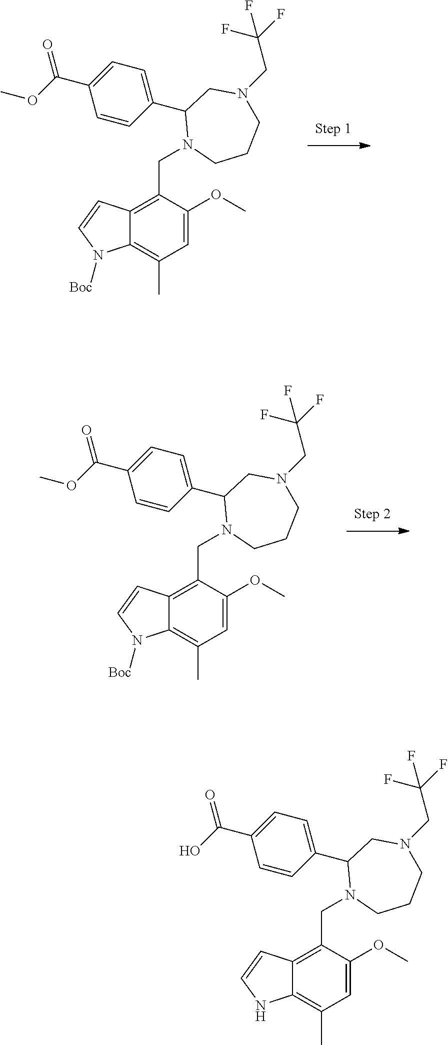 [SMILES: COC(=O)c1ccc(C2CN(CC(F)(F)F)CCCN2Cc2c(OC)cc(C)c3c2ccn3C(=O)OC(C)(C)C)cc1 ; COC(=O)c1ccc(C2CN(CC(F)(F)F)CCCN2Cc2c(OC)cc(C)c3c2ccn3C(=O)OC(C)(C)C)cc1 ; COc1cc(C)c2[nH]ccc2c1CN1CCCN(CC(F)(F)F)CC1c1ccc(C(=O)O)cc1]

Step 1. Enantiomer 2 of tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate was prepared according to the following procedure. Racemic tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate was separated by chiral SFC (100 mL 5% B) to afford enantiomer 2 of tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate (51 mg). Second eluting enantiomer (31697M UPC² 10m IF MA1) RT: 6.067 min; area % (238 nm): 93.1% ee; m/z=590.3 [M+H]⁺.

Step 2. Enantiomer 2 of 4-(1-((5-methoxy-7-methyl-1H-indol-4-yl)methyl)-4-(2,2,2-trifluoroethyl)-1,4-diazepan-2-yl)benzoic acid was prepared following General Experimental Procedure 8. Enantiomer 2 of tert-butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate (51 mg) gave enantiomer 2 of 4-(1-((5-methoxy-7-methyl-1H-indol-4-yl)methyl)-4-(2,2,2-trifluoroethyl)-1,4-diazepan-2-yl)benzoic acid (28.8 mg, 70%) as white solid. LCMS (31697 LCMS-5 C3) RT: 1.961 min; area % (215 nm): >99%; m/z=476.2 [M+H]⁺. ¹H-NMR (400 MHz, CD₃OD) δ 8.08 (d, J=8.0 Hz, 2H), 7.55 (d, J=8.0 Hz, 2H), 7.27 (d, J=3.1 Hz, 1H), 6.71 (s, 1H), 6.21 (d, J=3.2 Hz, 1H), 4.50 (br s, 1H), 4.35 (s, 2H), 3.71-3.33 (m, 8H), 3.17-2.95 (m, 3H), 2.49 (s, 3H), 2.21-2.16 (m, 1H), 2.08-2.04 (m, 1H).

### Example 103. 4-(4-(2-Fluoroethyl)-1-((5-methoxy-7-methyl-1H-indol-4-yl)methyl)-1,4-diazepan-2-yl)benzoic acid

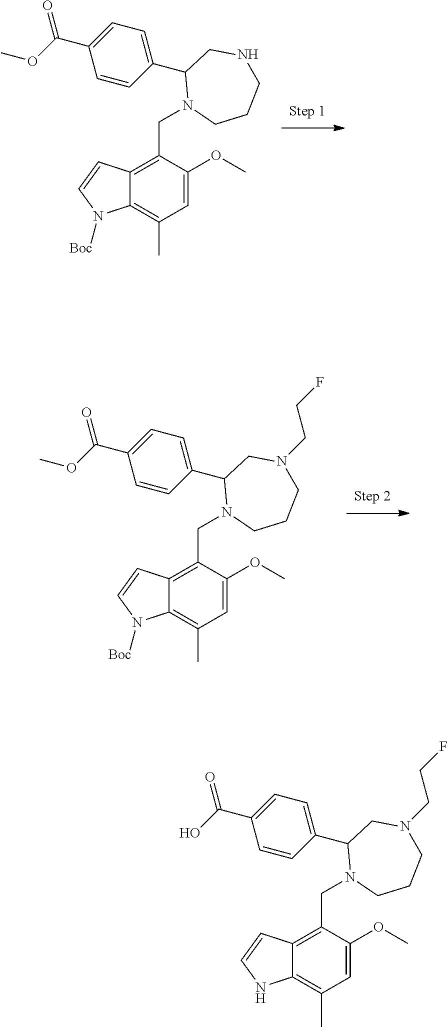 [SMILES: COC(=O)c1ccc(C2CNCCCN2Cc2c(OC)cc(C)c3c2ccn3C(=O)OC(C)(C)C)cc1 ; COC(=O)c1ccc(C2CN(CCF)CCCN2Cc2c(OC)cc(C)c3c2ccn3C(=O)OC(C)(C)C)cc1 ; COc1cc(C)c2[nH]ccc2c1CN1CCCN(CCF)CC1c1ccc(C(=O)O)cc1]

Step 1. tert-Butyl 4-((4-(2-fluoroethyl)-2-(4-(methoxycarbonyl)phenyl)-1,4-diazepan-1-yl)methyl)-5-methoxy-7-methyl-1H-indole-1-carboxylate was prepared following General Experimental Procedure 7. tert-Butyl 5-methoxy-4-((2-(4-(methoxycarbonyl)phenyl)-1,4-diazepan-1-yl)methyl)-7-methyl-1H-indole-1-carboxylate (70 mg, 0.14 mmol) and 1-Fluoro-2-iodo-ethane (23 μL, 0.28 mmol) gave tert-butyl 4-((4-(2-fluoroethyl)-2-(4-(methoxycarbonyl)phenyl)-1,4-diazepan-1-yl)methyl)-5-methoxy-7-methyl-1H-indole-1-carboxylate (44 mg, 58%). LCMS (LCMS-4 General 2 AB) RT: 2.699 min; area % (255 nm): 95.6%; m/z=554.4 [M+H]⁺.

Step 2. 4-(4-(2-Fluoroethyl)-1-((5-methoxy-7-methyl-1H-indol-4-yl)methyl)-1,4-diazepan-2-yl)benzoic acid was prepared following General Experimental Procedure 8. tert-Butyl 4-((4-(2-fluoroethyl)-2-(4-(methoxycarbonyl)phenyl)-1,4-diazepan-1-yl)methyl)-5-methoxy-7-methyl-1H-indole-1-carboxylate (43 mg, 78 μmol) gave 4-(4-(2-fluoroethyl)-1-((5-methoxy-7-methyl-1H-indol-4-yl)methyl)-1,4-diazepan-2-yl)benzoic acid (11 mg, 26%) as a white solid. LCMS (31697 LCMS-5 C3) RT: 1.596 min; area % (215 nm): 96.4%; m/z=440.2 [M+H]⁺. ¹H-NMR (400 MHz, CD₃OD) δ 8.13-8.05 (m, 2H), 7.62-7.55 (m, 2H), 7.29 (d, J=3.2 Hz, 1H), 6.72 (s, 1H), 6.22 (d, J=3.2 Hz, 1H), 4.58 (q, J=3.7, 2.8 Hz, 2H), 4.47 (t, J=4.7 Hz, 1H), 4.44-4.32 (m, 2H), 3.68 (s, 3H), 3.60-3.38 (m, 3H), 3.14 (dd, J=15.6, 2.9 Hz, 1H), 2.99 (h, J=4.6 Hz, 2H), 2.91 (t, J=4.7 Hz, 1H), 2.83 (dt, J=11.7, 5.6 Hz, 1H), 2.49 (d, J=0.7 Hz, 3H), 2.26-2.12 (m, 1H), 2.08-1.94 (m, 1H).

### Example 104. 4-(4-(2,2-Difluoroethyl)-1-((5-methoxy-7-methyl-1H-indol-4-yl)methyl)-1,4-diazepan-2-yl)benzoic acid

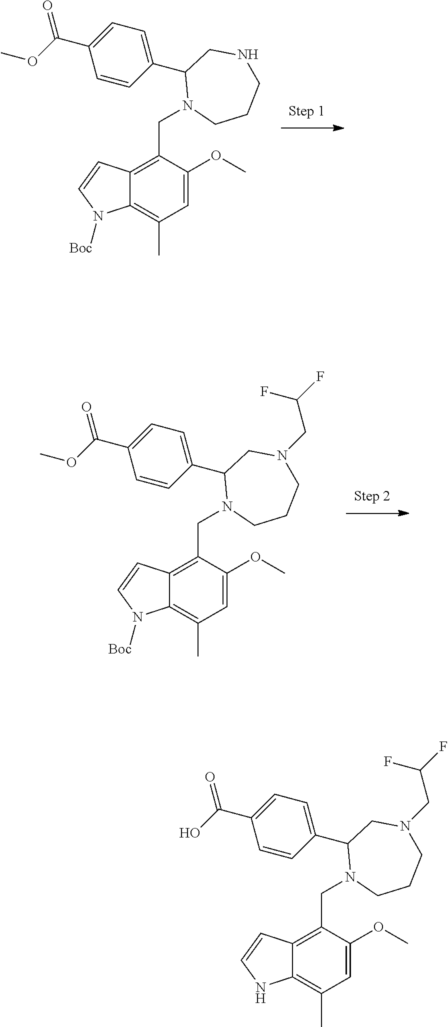 [SMILES: COC(=O)c1ccc(C2CNCCCN2Cc2c(OC)cc(C)c3c2ccn3C(=O)OC(C)(C)C)cc1 ; COC(=O)c1ccc(C2CN(CC(F)F)CCCN2Cc2c(OC)cc(C)c3c2ccn3C(=O)OC(C)(C)C)cc1 ; COc1cc(C)c2[nH]ccc2c1CN1CCCN(CC(F)F)CC1c1ccc(C(=O)O)cc1]

Step 1. tert-Butyl 4-((4-(2,2-difluoroethyl)-2-(4-(methoxycarbonyl)phenyl)-1,4-diazepan-1-yl)methyl)-5-methoxy-7-methyl-1H-indole-1-carboxylate was prepared following General Experimental Procedure 7. tert-Butyl 5-methoxy-4-((2-(4-(methoxycarbonyl)phenyl)-1,4-diazepan-1-yl)methyl)-7-methyl-1H-indole-1-carboxylate (70 mg, 0.14 mmol), and 2,2-difluoroethyl trifluoromethanesulphonate (55 μL, 0.41 mmol) gave tert-butyl 4-((4-(2,2-difluoroethyl)-2-(4-(methoxycarbonyl)phenyl)-1,4-diazepan-1-yl)methyl)-5-methoxy-7-methyl-1H-indole-1-carboxylate (30 mg, 38%). LCMS (LCMS-4 General 2 AB) RT: 2.833 min; area % (255 nm): 94.4%; m/z=572.4 [M+H]⁺.

Step 2. 4-(4-(2,2-Difluoroethyl)-1-((5-methoxy-7-methyl-1H-indol-4-yl)methyl)-1,4-diazepan-2-yl)benzoic acid was prepared following General Experimental Procedure 8. tert-Butyl 4-((4-(2,2-difluoroethyl)-2-(4-(methoxycarbonyl)phenyl)-1,4-diazepan-1-yl)methyl)-5-methoxy-7-methyl-1H-indole-1-carboxylate (30 mg, 50 μmol) gave 4-(4-(2,2-difluoroethyl)-1-((5-methoxy-7-methyl-1H-indol-4-yl)methyl)-1,4-diazepan-2-yl)benzoic acid (11 mg, 46%) as a white solid. LCMS (31697 LCMS-5 C3) RT: 1.765 min; area % (215 nm): 99.5%; m/z=458.2 [M+H]⁺. ¹H-NMR (400 MHz, CD₃OD) δ 8.14-8.05 (m, 2H), 7.62-7.53 (m, 2H), 7.28 (d, J=3.1 Hz, 1H), 6.72 (s, 1H), 6.21 (d, J=3.2 Hz, 1H), 5.95 (tt, J=55.9, 4.1 Hz, 1H), 4.58-4.55 (m, 3H), 4.39 (s, 2H), 3.66 (s, 3H), 3.56-3.48 (m, 3H), 3.15-2.96 (m, 4H), 2.92 (dt, J=11.6, 5.5 Hz, 1H), 2.49 (s, 3H), 2.21-2.17 (m, 1H), 2.13-1.98 (m, 1H).

### Example 105. 4-(1-((5-methoxy-7-methyl-1H-indol-4-yl)methyl)-4-(2,2,2-trifluoroethyl)-1,4-diazepan-2-yl)benzoic acid

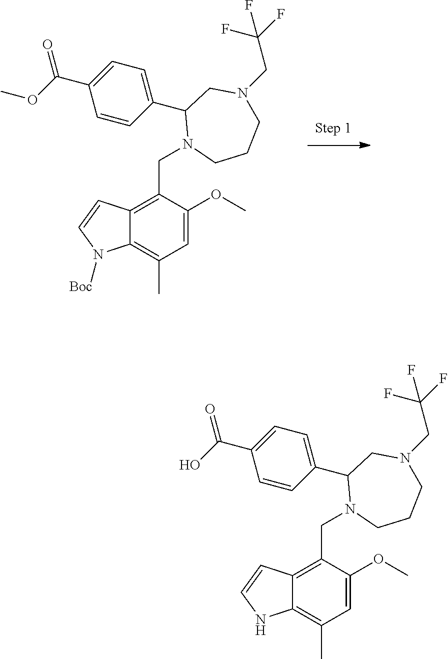 [SMILES: COC(=O)c1ccc(C2CN(CC(F)(F)F)CCCN2Cc2c(OC)cc(C)c3c2ccn3C(=O)OC(C)(C)C)cc1 ; COc1cc(C)c2[nH]ccc2c1CN1CCCN(CC(F)(F)F)CC1c1ccc(C(=O)O)cc1]

Step 1. 4-(1-((5-methoxy-7-methyl-1H-indol-4-yl)methyl)-4-(2,2,2-trifluoroethyl)-1,4-diazepan-2-yl)benzoic acid was prepared following General Experimental Procedure 8. tert-Butyl 5-methoxy-4-((2-(4-(methoxycarbonyl)phenyl)-4-(2,2,2-trifluoroethyl)-1,4-diazepan-1-yl)methyl)-7-methyl-1H-indole-1-carboxylate (35 mg, 59 μmol) gave 4-(1-((5-methoxy-7-methyl-1H-indol-4-yl)methyl)-4-(2,2,2-trifluoroethyl)-1,4-diazepan-2-yl)benzoic acid (14 mg, 50%) as a white solid. LCMS (31697 LCMS-5 C3) RT: 1.965 min; area % (215 nm): 99.9%; m/z=476.2 [M+H]⁺. ¹H-NMR (400 MHz, CD₃OD) δ 8.13-8.05 (m, 2H), 7.60-7.51 (m, 2H), 7.28 (d, J=3.1 Hz, 1H), 6.71 (s, 1H), 6.21 (d, J=3.2 Hz, 1H), 4.70-4.47 (m, 2H), 4.37 (s, 2H), 3.70-3.32 (m, 8H), 3.19-2.95 (m, 3H), 2.49 (s, 3H), 2.24-2.16 (m, 1H), 2.13-2.01 (m, 1H).

### Example 106. 4-(4-((5-Methoxy-7-methyl-1H-indol-4-yl)methyl)-1-methyl-7-oxo-1,4-diazepan-5-yl)benzoic acid

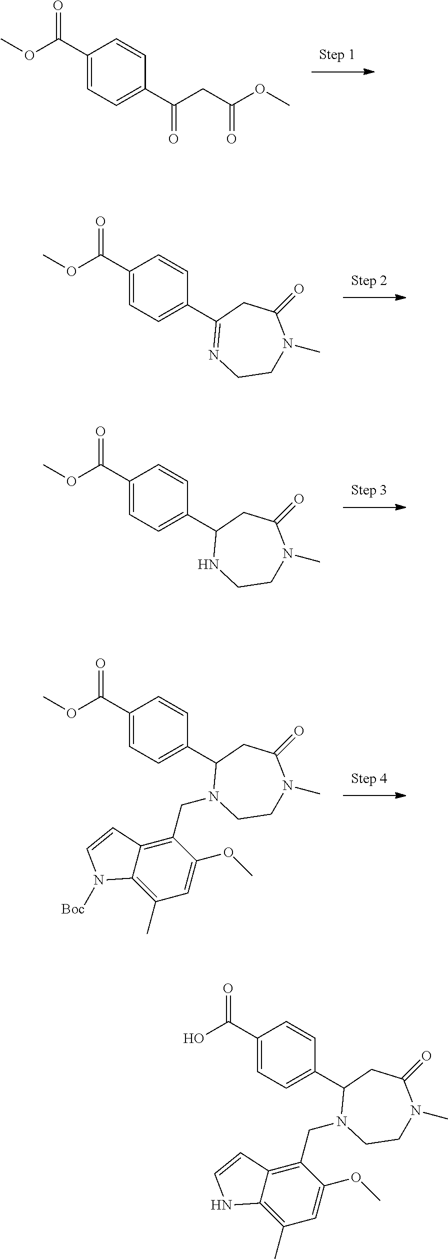

Step 1. Methyl 4-(1-methyl-7-oxo-2,3,6,7-tetrahydro-1H-1,4-diazepin-5-yl)benzoate was prepared according the following procedure. Methyl 4-(3-methoxy-3-oxopropanoyl)benzoate (200 mg, 1.0 eq, 847 μmol) and N1-methylethane-1,2-diamine (62.8 mg, 1.0 eq, 847 μmol) were dissolved in p-xylene (20 mL) and the reaction mixture stirred at 160° C. for 10 hours. The reaction was worked-up; the precipitate was collected by filtration, the filter cake washed with Et₂O, and the residue dried under reduced pressure to afford methyl 4-(1-methyl-7-oxo-2,3,6,7-tetrahydro-1H-1,4-diazepin-5-yl)benzoate (100 mg, 45%) as a brown solid. LCMS (General 3 Basic) RT: 0.86 min; area % (255 nm): 95%; m/z=261.2 [M+H]⁺.

Step 2. Methyl 4-(1-methyl-7-oxo-1,4-diazepan-5-yl)benzoate was prepared according to the following procedure. Methyl 4-(1-methyl-7-oxo-2,3,4,7-tetrahydro-1H-1,4-diazepin-5-yl)benzoate (67 mg, 1.0 eq, 0.26 mmol) was suspended in EtOAc (3 mL). Then 10% Pd/C (27 mg, 0.1 eq, 26 μmol) was added and the reaction mixture stirred under H₂ atmosphere (balloon) at 60° C. for 15 h. The reaction was worked-up; filtered over Celite® and the filtrate concentrated under reduced pressure to afford crude Methyl 4-(1-methyl-7-oxo-1,4-diazepan-5-yl)benzoate (80 mg). LCMS (General 3 Basic) RT: 0.62 min; area % (254 nm): 88%; m/z=263.2 [M+H]⁺.

Step 3. tert-Butyl 5-methoxy-4-((7-(4-(methoxycarbonyl)phenyl)-4-methyl-5-oxo-1,4-diazepan-1-yl)methyl)-7-methyl-1H-indole-1-carboxylate was prepared with the following procedure. Crude methyl 4-(7-oxo-1,4-diazepan-5-yl)benzoate (56 mg), tert-butyl 4-formyl-5-methoxy-7-methyl-1H-indole-1-carboxylate (120 mg, 2.0 eq, 0.43 mmol) and acetic acid (13 μL, 1.1 eq, 0.23 mmol) were dissolved in DCE (5 mL). After stirring at 20° C. for 1 hour, sodium triacetoxyborohydride (0.18 g, 4.0 eq, 0.85 mmol) was added and the mixture stirred at 20° C. for 15 h. The reaction was worked-up; washed with sat. aq. NaHCO₃ (5 mL), H₂O (10 mL), dried over Na2SO₄, filtered, and concentrated under reduced pressure. The crude was purified by automated normal phase FCC to afford tert-butyl 5-methoxy-4-((7-(4-(methoxycarbonyl)phenyl)-4-methyl-5-oxo-1,4-diazepan-1-yl)methyl)-7-methyl-1H-indole-1-carboxylate (20 mg, 17%). LCMS (General 3 Basic) RT: 2.03 min; area % (254 nm): 90%; m/z=536.3 [M+H]⁺

Step 4. 4-(4-((5-Methoxy-7-methyl-1H-indol-4-yl)methyl)-1-methyl-7-oxo-1,4-diazepan-5-yl)benzoic acid was prepared following General Experimental Procedure 8. tert-Butyl 5-methoxy-4-((7-(4-(methoxycarbonyl)phenyl)-4-methyl-5-oxo-1,4-diazepan-1-yl)methyl)-7-methyl-1H-indole-1-carboxylate (20 mg, 37 μmol) gave 4-(4-((5-methoxy-7-methyl-1H-indol-4-yl)methyl)-1-methyl-7-oxo-1,4-diazepan-5-yl)benzoic acid (8.8 mg, 56%). LCMS (31697 LCMS-5 C3) RT: 1.659 min; area % (215 nm): 93.8%; m/z=422.2 [M+H]⁺. ¹H-NMR (400 MHz, CD₃OD) δ 8.06 (d, J=8.0 Hz, 2H), 7.64 (d, J=8.1 Hz, 2H), 7.21 (d, J=3.1 Hz, 1H), 6.69 (d, J=0.9 Hz, 1H), 6.39 (d, J=3.1 Hz, 1H), 3.88-3.66 (m, 6H), 3.59 (d, J=12.1 Hz, 1H), 3.45-3.33 (m, 2H), 3.18 (dd, J=14.2, 7.1 Hz, 1H), 2.96 (s, 3H), 2.70-2.57 (m, 2H), 2.46 (d, J=0.8 Hz, 3H).

### Example 107. 4-(4-((5-Methoxy-7-methyl-1H-indol-4-yl)methyl)-7-oxo-1,4-diazepan-5-yl)benzoic acid

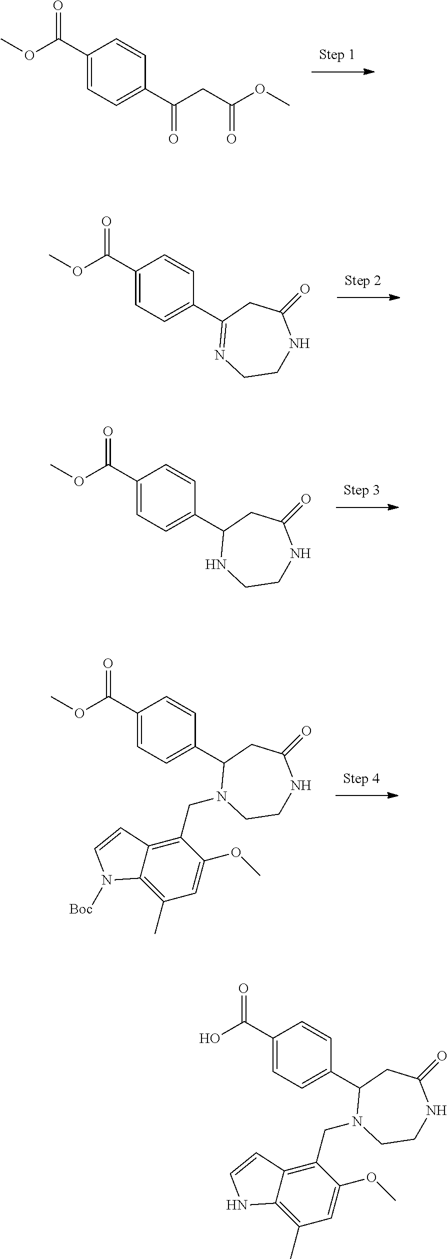

Step 1. Methyl 4-(7-oxo-2,3,6,7-tetrahydro-1H-1,4-diazepin-5-yl)benzoate was prepared according to the following procedure. Methyl 4-(3-methoxy-3-oxopropanoyl)benzoate (200 mg, 1.0 eq, 847 μmol) and ethane-1,2-diamine (50.9 mg, 1.0 eq, 847 μmol) were dissolved in p-xylene (20 mL) and the reaction mixture was stirred at 160° C. for 10 hours. The reaction was worked-up; concentrated under reduced pressure and the brown residue purified by automated normal phase FCC to afford crude methyl 4-(7-oxo-2,3,6,7-tetrahydro-1H-1,4-diazepin-5-yl)benzoate (100 mg, 48%) as a yellow oil. LCMS (General 3 Basic) RT: 0.72 min; area % (254 nm): 89%; m/z=247.2 [M+H]⁺.

Step 2. Methyl 4-(7-oxo-1,4-diazepan-5-yl)benzoate was prepared according to the following procedure. Methyl 4-(7-oxo-2,3,6,7-tetrahydro-1H-1,4-diazepin-5-yl)benzoate (66 mg, 1.0 eq, 268 μmol) and 10% Pd/C (43 mg, 0.15 eq, 40.2 μmol) were suspended in EtOAc (5 mL). The reaction mixture was stirred under H₂ atmosphere (balloon) at 65° C. for 72 h. The reaction was worked-up; filtered over Celite® and the filtrate concentrated under reduced pressure to afford crude methyl 4-(7-oxo-1,4-diazepan-5-yl)benzoate (40 mg). LCMS (General 3 Basic) RT: 0.56 min; area % (254 nm): 54%; m/z=249.2 [M+H]⁺.

Step 3. tert-Butyl 5-methoxy-4-((7-(4-(methoxycarbonyl)phenyl)-5-oxo-1,4-diazepan-1-yl)methyl)-7-methyl-1H-indole-1-carboxylate was prepared with the following procedure. Methyl 4-(7-oxo-1,4-diazepan-5-yl)benzoate (40 mg, 1.0 eq, 0.16 mmol), tert-butyl 4-formyl-5-methoxy-7-methyl-1H-indole-1-carboxylate (93 mg, 2.0 eq, 0.32 mmol) and acetic acid (10 μL, 1.1 eq, 0.18 mmol) were dissolved in DCE (5 mL). After stirring at 20° C. for 1 hour, sodium triacctoxyborohydride (0.14 g, 4.0 eq, 0.64 mmol) was added and the mixture stirred at 20° C. for 15 h. The reaction was worked-up; washed with sat. aq. NaHCO₃ (5 mL), H₂O (10 mL), dried over Na₂SO₄, filtered, and concentrated under reduced pressure. The crude was purified by automated normal phase FCC to afford tert-butyl 5-methoxy-4-((7-(4-(methoxycarbonyl)phenyl)-5-oxo-1,4-diazepan-1-yl)methyl)-7-methyl-1H-indole-1-carboxylate (25 mg, 30%). LCMS (General 3 Basic) RT: 1.94 min; area % (254 nm): 82%; m/z=522.2 [M+H]⁺.

Step 4. 4-(4-((5-Methoxy-7-methyl-1H-indol-4-yl)methyl)-7-oxo-1,4-diazepan-5-yl)benzoic acid was prepared following General Experimental Procedure 8. tert-Butyl 5-methoxy-4-((7-(4-(methoxycarbonyl)phenyl)-5-oxo-1,4-diazepan-1-yl)methyl)-7-methyl-1H-indole-1-carboxylate (25 mg, 48 μmol) gave 4-(4-((5-methoxy-7-methyl-1H-indol-4-yl)methyl)-7-oxo-1,4-diazepan-5-yl)benzoic acid (6.99 mg product, 36%) as a white solid. LCMS (31697 LCMS-5 C3) RT: 1.503 min; area % (215 nm): 94.1%; m/z=408.0 [M+H]⁺. ¹H-NMR (400 MHz, CD₃OD) δ 8.08-7.99 (m, 2H), 7.64 (d, J=8.3 Hz, 2H), 7.21 (d, J=3.1 Hz, 1H), 6.69 (s, 1H), 6.40 (d, J=3.1 Hz, 1H), 3.88 (d, J=11.8 Hz, 2H), 3.77 (s, 3H), 3.65 (d, J=12.1 Hz, 1H), 3.34-3.21 (m, 3H), 3.20-3.10 (m, 1H), 2.70 (dd, J=14.9, 1.5 Hz, 1H), 2.66-2.56 (m, 1H), 2.46 (s, 3H).

### Example 108. 4-(4-((5-Methoxy-7-methyl-1H-indol-4-yl)methyl)-1,7-dimethyl-1,4-diazepan-5-yl)benzoic acid

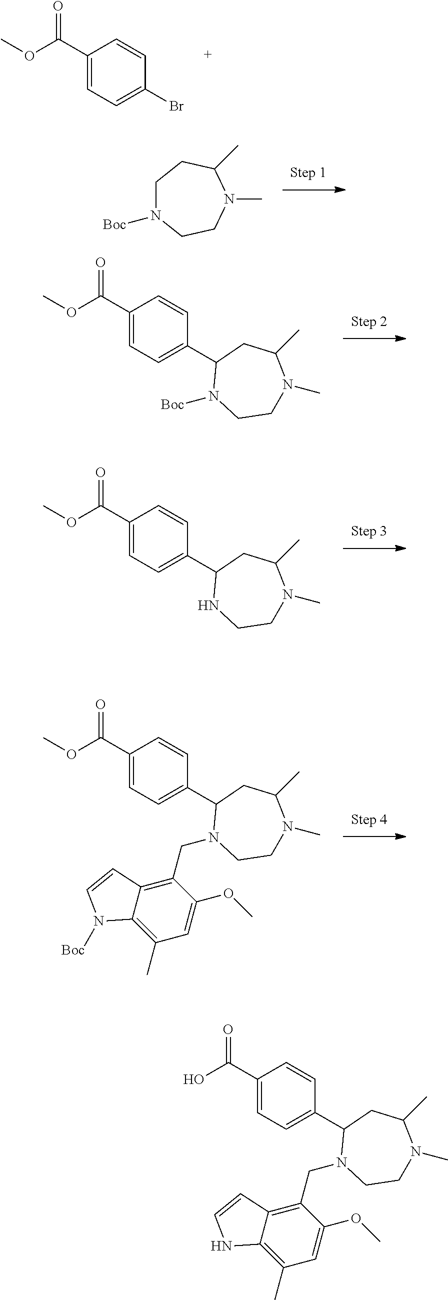

Step 1. tert-Butyl 7-(4-(methoxycarbonyl)phenyl)-4,5-dimethyl-1,4-diazepane-1-carboxylate was prepared with the following procedure. In a flame-dried, 100 mL 3-necked round bottom flask, under nitrogen, a solution of tert-butyl 4,5-dimethyl-1,4-diazepane-1-carboxylate (1.56 g, 1.47 eq, 6.83 mmol), TMEDA (1.12 mL, 1.6 eq, 7.440 mmol) and anhydrous Et₂O (25 mL) was cooled to −78° C. and 1.3 M sec-butyllithium in cyclohexan/hexane (5.72 mL, 1.6 eq, 7.440 mmol) was added dropwise. The reaction mixture was allowed to stir at this temperature for 2 hours before 0.7 M ZnCl₂ in THF (10.6 mL, 1.6 eq, 7.44 mmol) was added. The reaction mixture was warm to 20° C. and allowed to stir at this temperature for 2 hours. Meanwhile a solution of Pd(PPh₃)₄ (322 mg, 0.06 Eq, 279 μmol) and 2-dicyclohexylphosphino-2,6-dimethoxy-1,1-biphenyl (234.8 mg, 0.123 Eq, 572.0 μmol) in dry toluene (60 mL) was prepared. The catalyst solution was added to the piperidinyl zinc reagent followed by methyl 4-bromobenzoate (1.000 g, 1.0 eq, 4.650 mmol). The Et₂O was removed by Dean-Stark distillation and the resulting mixture was allowed to stir at 110° C. for 15 h. The reaction was worked-up; cooled down to 20° C. and concentrated under reduced pressure. The residue was purified by automated normal phase FCC to afford tert-butyl 7-(4-(methoxycarbonyl)phenyl)-4,5-dimethyl-1,4-diazepane-1-carboxylate (225 mg, 15%). LCMS (LCMS-4 General 3 AB) RT: 1.863 min; area % (254 nm): 85.9%; m/z=363.4 [M+H]⁺.

Step 2. Methyl 4-(1,7-dimethyl-1,4-diazepan-5-yl)benzoate was prepared according to the following procedure. tert-Butyl 7-(4-(methoxycarbonyl)phenyl)-4,5-dimethyl-1,4-diazepane-1-carboxylate (185 mg, 1.0 eq, 510 μmol) was dissolved in 4 N HCl in dioxane (2 mL, 8 mmol). The reaction mixture was stirred at 20° C. for 15 h. The reaction mixture was worked-up; neutralized with 2 N aq. NaOH to pH around 7. Then extracted with DCM (2× 10 mL). The combined organic layers were dried over Na₂SO₄, filtered, and concentrated under reduced pressure to afford crude methyl 4-(1,7-dimethyl-1,4-diazepan-5-yl)benzoate (80 mg) as a brown oil. LCMS (LCMS-4 General 2 AB) RT: 0.49 min; area % (254 nm): 83%; m/z=263.2 [M+H]⁺.

Step 3. tert-Butyl 5-methoxy-4-((7-(4-(methoxycarbonyl)phenyl)-4,5-dimethyl-1,4-diazepan-1-yl)methyl)-7-methyl-1H-indole-1-carboxylate was prepared with the following procedure. Methyl 4-(1,7-dimethyl-1,4-diazepan-5-yl)benzoate (80 mg, 1.0 eq, 0.30 mmol) was dissolved in DCE (5 mL). Then tert-butyl 4-formyl-5-methoxy-7-methyl-1H-indole-1-carboxylate (0.13 g, 1.5 eq, 0.46 mmol) and AcOH (19 μL, 1.1 Eq, 0.34 mmol) were added and the mixture stirred at 20° C. for 1 hour. Then sodium triacetoxyborohydride (0.26 g, 4.0 eq, 1.2 mmol) was added. The reaction mixture was stirred at 20° C. for 15 h. The reaction was worked-up; washed with sat. aq. NaHCO₃ (5 mL), H₂O (5 mL), dried over Na₂SO₄, filtred, and concentrated under reduced pressure. The residue was purified by automated normal phase FCC to afford tert-butyl 5-methoxy-4-((7-(4-(methoxycarbonyl)phenyl)-4,5-dimethyl-1,4-diazepan-1-yl)methyl)-7-methyl-1H-indole-1-carboxylate (120 mg, 73%) as a light yellow oil. LCMS (LCMS-4 General 2 AB) RT: 2.20 min; area % (254 nm): 89%; m/z=536.3 [M+H]⁺.

Step 4. 4-(4-((5-Methoxy-7-methyl-1H-indol-4-yl)methyl)-1,7-dimethyl-1,4-diazepan-5-yl)benzoic acid was prepared following General Experimental Procedure 8. tert-Butyl 5-methoxy-4-((7-(4-(methoxycarbonyl)phenyl)-4,5-dimethyl-1,4-diazepan-1-yl)methyl)-7-methyl-1H-indole-1-carboxylate (120 mg, 224 μmol) gave 4-(4-((5-methoxy-7-methyl-1H-indol-4-yl)methyl)-1,7-dimethyl-1,4-diazepan-5-yl)benzoic acid (76 mg, 80%) product as light yellow solid. LCMS (31697 LCMS-5 C3) RT: 1.444 min and 1.538 min (diastereomeric ratio 6.5:1); area % (215 nm): 97.4%; m/z=422.2 [M+H]⁺.

### Example 109. 4-(7-(Difluoromethyl)-4-((5-methoxy-7-methyl-1H-indol-4-yl)methyl)-1-methyl-1,4-diazepan-5-yl)benzoic acid

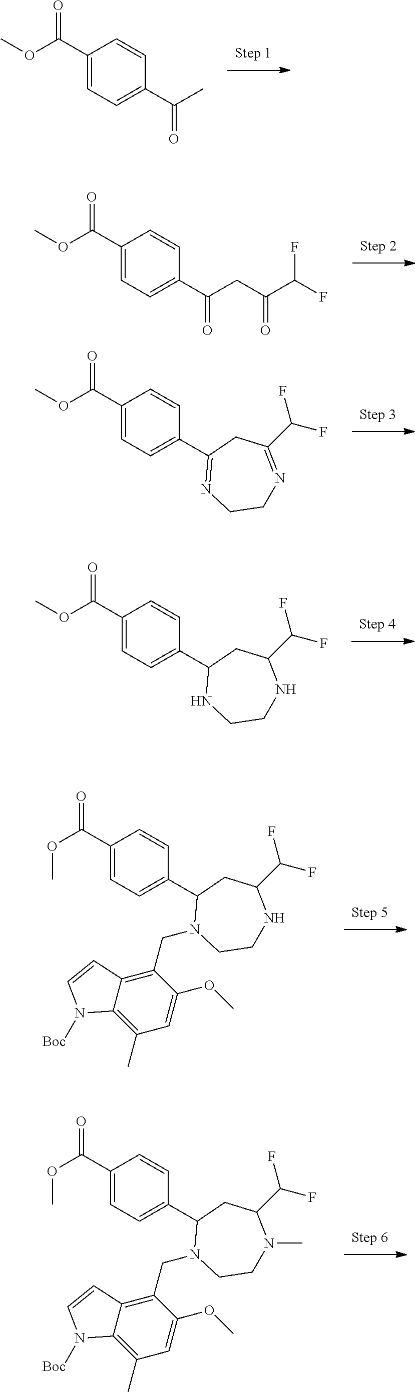

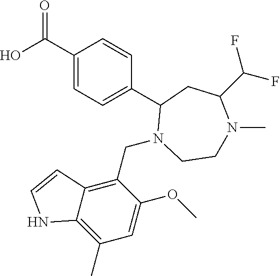 [SMILES: COc1cc(C)c2[nH]ccc2c1CN1CCN(C)C(C(F)F)CC1c1ccc(C(=O)O)cc1]

Step 1: Methyl 4-(4,4-difluoro-3-oxobutanoyl)benzoate was prepared according to the following procedure. Methyl 4-acetylbenzoate (1.00 g, 1.0 eq, 5.61 mmol) was dissolved in THF (25 mL) and cooled to 0° C. Then 60 wt. % NaH dispersion in mineral oil (0.29 g, 1.3 eq, 7.30 mmol) was added and the mixture stirred for 30 min at 0° C. Then methyl 2,2-difluoroacetate (537 μL, 1.1 eq, 6.17 mmol) was added dropwise and the mixture stirred for 2 hours. The reaction was worked-up; concentrated under reduced pressure and dissolved residue in DCM (20 mL). The organic layers were washed with 5% aq. HCl, H₂O (2×20 mL), brine, dried over Na₂SO₄, and concentrated under reduced pressure to afford methyl 4-(4,4-difluoro-3-oxobutanoyl)benzoate (1.44 g, quant.) as a light yellow solid. LCMS (LCMS-4 General 3 AB) RT: 0.80 min; area % (254 nm): 96%; m/z=255.0 [M−H]⁻.

Step 2. Methyl 4-(7-(difluoromethyl)-3,6-dihydro-2H-1,4-diazepin-5-yl)benzoate was prepared according to the following procedure. Methyl 4-(4,4-difluoro-3-oxobutanoyl)benzoate (500 mg, 1.0 eq, 1.95 mmol) was dissolved in p-xylene (20 mL). Then ethylenediamine (130 μL, 1.0 eq, 1.95 mmol) was added and the resulting mixture stirred at 160° C. for 11 hours. The reaction was worked-up; cooled to room temperature and concentrated undere reduced pressure. The residue was purified by automated normal phase FCC to afford methyl 4-(7-(difluoromethyl)-3,6-dihydro-2H-1,4-diazepin-5-yl)benzoate (332 mg, 59%) as a brown oil. LCMS (LCMS-4 General 3 AB) RT: 1.36 min; area % (254 nm): 86%; m/z=281.1 [M+H]⁺.

Step 3. Methyl 4-(7-(difluoromethyl)-1,4-diazepan-5-yl)benzoate was prepared with the following procedure. Methyl 4-(7-(difluoromethyl)-3,6-dihydro-2H-1,4-diazepin-5-yl)benzoate (100 mg, 1.0 eq, 357 μmol) was dissolved in EtOAc (10 mL). Then 10% Pd/C (38.0 mg, 0.1 Eq, 35.7 μmol) was added and the reaction mixture was stirred under H₂ atmosphere (balloon) at 20° C. for 15 h. The reaction was worked-up; filtered through Celite® and the filtrate concentrated under reduced pressure. The residue was purified by automated normal phase FCC to afford methyl 4-(7-(difluoromethyl)-1,4-diazepan-5-yl)benzoate (68 mg, 67%) as a light yellow oil. LCMS (LCMS-4 General 3 AB) RT: 1.06 min and 1.08 min (diastereomeric ratio 1:1); area % (254 nm): 91.7%; m/z=285.1 [M+H]⁺.

Step 4. tert-Butyl 4-((5-(difluoromethyl)-7-(4-(methoxycarbonyl)phenyl)-1,4-diazepan-1-yl)methyl)-5-methoxy-7-methyl-1H-indole-1-carboxylate was prepared with the following procedure. Methyl 4-(7-(difluoromethyl)-1,4-diazepan-5-yl)benzoate (60.0 mg, 1.0 eq, 211 μmol), tert-butyl 4-formyl-5-methoxy-7-methyl-1H-indole-1-carboxylate (61.1 mg, 1.0 eq, 211 μmol) and acetic acid (13.3 μL, 1.1 eq, 232 μmol) were stirred in DCE (2 mL) at 20° C. for 1 hour. Then sodium triacetoxyborohydride (179 mg, 4.0 eq, 844 μmol) was added and continued stirring at 20° C. for 15 h. The reaction was worked-up; diluted with DCM, washed with sat. aq. NaHCO₃ (5 mL), H₂O (10 mL), dried over Na₂SO₄, and concentrated under reduced pressure. The residue was purified by automated normal phase FCC to afford tert-butyl 4-((5-(difluoromethyl)-7-(4-(methoxycarbonyl)phenyl)-1,4-diazepan-1-yl)methyl)-5-methoxy-7-methyl-1H-indole-1-carboxylate (80 mg, 68%) as light yellow oil. LCMS (LCMS-4 General 2 AB) RT: 1.96 min and 2.00 min (diastereomeric ratio 1:1); area % (254 nm): 91%; m/z=558.3 [M+H]⁺.

Step 5. tert-Butyl 4-((5-(difluoromethyl)-7-(4-(methoxycarbonyl)phenyl)-4-methyl-1,4-diazepan-1-yl)methyl)-5-methoxy-7-methyl-1H-indole-1-carboxylate was prepared with the following procedure. tert-Butyl 4-((5-(difluoromethyl)-7-(4-(methoxycarbonyl)phenyl)-1,4-diazepan-1-yl)methyl)-5-methoxy-7-methyl-1H-indole-1-carboxylate (80 mg, 1.0 eq, 0.14 mmol) was dissolved in DCE (3 mL). Then acetic acid (9.0 μL, 1.1 Eq, 0.16 mmol) and 37 wt. % aq. formaldehyde (21 μL, 2.0 eq, 0.29 mmol) were added and the reaction mixture stirred at 20° C. for 30 min. Then sodium triacetoxyborohydride (0.12 g, 4.0 eq, 0.57 mmol) was added and the resulting mixture stirred at 20° C. for 3 hours. The reaction was worked-up; diluted with DCM (10 mL), washed with sat. aq. NaHCO₃ (15 mL), H₂O (15 mL), dried over Na₂SO₄, concentrated under reduced pressure. The residue was purified by automated normal phase FCC to afford tert-butyl 4-((5-(difluoromethyl)-7-(4-(methoxycarbonyl)phenyl)-4-methyl-1,4-diazepan-1-yl)methyl)-5-methoxy-7-methyl-1H-indole-1-carboxylate (69 mg, 84%) as a white solid. LCMS (LCMS-4 General 2 AB) RT: 2.20 min and 2.23 min (diastereomeric ratio 1:1); area % (254 nm): 88.3%; m/z=572.3 [M+H]⁺.

Step 6. 4-(7-(Difluoromethyl)-4-((5-methoxy-7-methyl-1H-indol-4-yl)methyl)-1-methyl-1,4-diazepan-5-yl)benzoic acid was prepared following General Experimental Procedure 8. tert-Butyl 4-((5-(difluoromethyl)-7-(4-(methoxycarbonyl)phenyl)-4-methyl-1,4-diazepan-1-yl)methyl)-5-methoxy-7-methyl-1H-indole-1-carboxylate (70 mg, 120 μmol) gave 4-(7-(difluoromethyl)-4-((5-methoxy-7-methyl-1H-indol-4-yl)methyl)-1-methyl-1,4-diazepan-5-yl)benzoic acid (22.3 mg, 40%) as a white solid. LCMS (31697 LCMS-5 C3) RT: 1.744 min and 1.807 min (diastereomeric ratio 1:1); area % (215 nm): 95.1%; m/z=458.2 [M+H]⁺.

### Example 110. 4-(4-((5-Methoxy-7-methyl-1H-indol-4-yl)methyl)-1-methyl-7-(trifluoromethyl)-1,4-diazepan-5-yl)benzoic acid

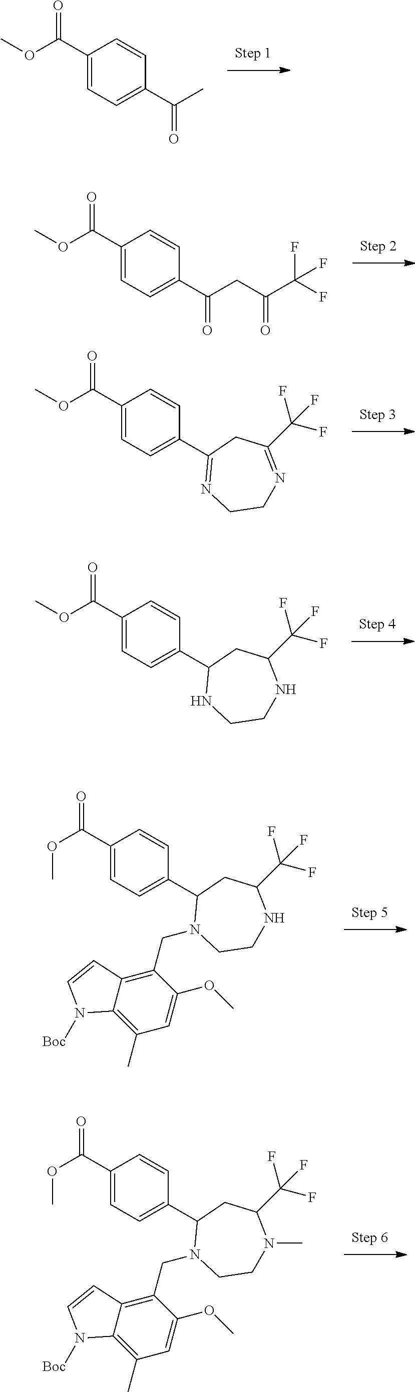

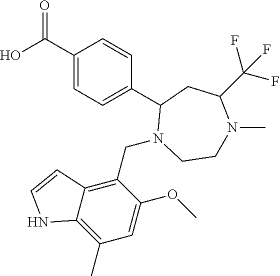 [SMILES: COc1cc(C)c2[nH]ccc2c1CN1CCN(C)C(C(F)(F)F)CC1c1ccc(C(=O)O)cc1]

Step 1. Methyl 4-(4,4,4-trifluoro-3-oxobutanoyl)benzoate was prepared according to the following procedure. Methyl 4-acetylbenzoate (2.00 g, 1.0 eq, 11.2 mmol) was dissolved in THF (30 mL) and cooled to 0° C. Then 60 wt. % NaH dispersion in oil (0.58 g, 1.3 eq, 14.6 mmol) was added and the mixture stirred at 0° C. for 30 min. Methyl trifluoroacetate (1.24 mL, 1.1 eq, 12.3 mmol) was added dropwise and the reaction mixture stirred for 2 hours. The reaction was worked-up; concentrated under reduced pressure and dissolved residue in DCM (20 mL). The organic layers were washed with 5% aq. HCl, H₂O (2×20 mL), brine, dried over Na₂SO₄, and concentrated under reduced pressure to afford methyl 4-(4,4,4-trifluoro-3-oxobutanoyl)benzoate (3.02 g, quant.) as a light yellow solid. LCMS (LCMS-4 General 3 AB) RT: 1.03 min; area % (254 nm): 97%; m/z=273.1 [M−H]⁻.

Step 2. Methyl 4-(7-(trifluoromethyl)-3,6-dihydro-2H-1,4-diazepin-5-yl)benzoate was prepared with the following procedure. Methyl 4-(4,4,4-trifluoro-3-oxobutanoyl)benzoate (500 mg, 1.0 eq, 1.82 mmol) was dissolved in p-xylene (20 mL). Then ethylenediamine (122 μL, 1.0 eq, 1.82 mmol) was added and the resulting suspension was stirred at 160° C. for 11 hours. The reaction was worked-up; cooled to room temperature and concentrated under reduced pressure. The residue was purified by automated normal phase FCC to afford methyl 4-(7-(trifluoromethyl)-3,6-dihydro-2H-1,4-diazepin-5-yl)benzoate (118 mg, 22%) as a brown oil. LCMS (LCMS-4 General 3 AB) RT: 1.45 min; area % (254 nm): 89%; m/z=299.1 [M+H]⁺.

Step 3. Methyl 4-(7-(trifluoromethyl)-1,4-diazepan-5-yl)benzoate was prepared with the following procedure. Methyl 4-(7-(trifluoromethyl)-3,6-dihydro-2H-1,4-diazepin-5-yl)benzoate (100 mg, 1.0 eq, 335 μmol) was dissolved in EtOAc (10 mL). Then 10% Pd/C (35.7 mg, 0.1 eq, 33.5 μmol) was added and the resulting mixture stirred under H₂ atmosphere (balloon) at 20° C. for 15 h. The reaction was worked-up; filtered through Celite® and the filtrate concentrated under reduced pressure. The residue was purified by automated normal phase FCC to afford methyl 4-(7-(trifluoromethyl)-1,4-diazepan-5-yl)benzoate (64 mg, 63%) product as abrown oil. LCMS (LCMS-4 General 3 AB) RT: 1.25 min; area % (254 nm): 76%; m/z=303.1 [M+H]⁺.

Step 4. tert-Butyl 5-methoxy-4-((7-(4-(methoxycarbonyl)phenyl)-5-(trifluoromethyl)-1,4-diazepan-1-yl)methyl)-7-methyl-1H-indole-1-carboxylate was prepared with the following procedure. Methyl 4-(7-(trifluoromethyl)-1,4-diazepan-5-yl)benzoate (60.0 mg, 1.0 eq, 198 μmol), tert-butyl 4-formyl-5-methoxy-7-methyl-1H-indole-1-carboxylate (57.4 mg, 1.0 eq, 198 μmol) and acetic acid (12.5 μL, 1.1 eq, 218 μmol) were stirred in DCE (3 mL) at 20° C. for 1 h. Then sodium triacetoxyborohydride (168 mg, 4.0 eq, 794 μmol) was added and the mixture stirred at 20° C. for 15 h. The reaction was worked-up; diluted with DCM, washed with sat. aq. NaHCO₃ (5 mL), H₂O (10 mL), dried over Na₂SO₄, and concentrated under reduced pressure. The residue was purified by automated normal phase FCC to afford tert-butyl 5-methoxy-4-((7-(4-(methoxycarbonyl)phenyl)-5-(trifluoromethyl)-1,4-diazepan-1-yl)methyl)-7-methyl-1H-indole-1-carboxylate (79 mg, 69%) as a light yellow oil. LCMS (LCMS-4 General 2 AB) RT: 2.10 min and 2.13 min (diastereomeric ratio 1:1); area % (254 nm): 69.2%; m/z=576.3 [M+H]⁺.

Step 5. tert-Butyl 5-methoxy-4-((7-(4-(methoxycarbonyl)phenyl)-4-methyl-5-(trifluoromethyl)-1,4-diazepan-1-yl)methyl)-7-methyl-1H-indole-1-carboxylate was prepared with the following procedure. tert-Butyl 5-methoxy-4-((7-(4-(methoxycarbonyl)phenyl)-5-(trifluoromethyl)-1,4-diazepan-1-yl)methyl)-7-methyl-1H-indole-1-carboxylate (79 mg, 1.0 eq, 0.14 mmol) was dissolved in DCE (3 mL). Then acetic acid (8.6 μL, 1.1 eq, 0.15 mmol) and 37 wt. % aq. formaldehyde (20 μL, 2.0 eq, 0.27 mmol) were added and stirred at 20° C. for 1 hour. Then sodium triacetoxyborohydride (0.12 g, 4.0 eq, 0.55 mmol) was added and the reaction mixture stirred at 20° C. for 2 hours. The reaction was worked-up; diluted with DCM (10 mL), washed with sat. aq. NaHCO₃ (15 mL), H₂O (15 mL), dried over Na₂SO₄, concentrated under reduced pressure. The residue was purified by automated normal phase FCC to afford tert-butyl 5-methoxy-4-((7-(4-(methoxycarbonyl)phenyl)-4-methyl-5-(trifluoromethyl)-1,4-diazepan-1-yl)methyl)-7-methyl-1H-indole-1-carboxylate (55 mg, 68%) as a white solid. LCMS (LCMS-4 General 2 AB) RT: 2.35 min and 2.39 min (diastereomeric ratio 1:1); area % (254 nm): 91%; m/z=590.3 [M+H]⁺.

Step 6. 4-(4-((5-Methoxy-7-methyl-1H-indol-4-yl)methyl)-1-methyl-7-(trifluoromethyl)-1,4-diazepan-5-yl)benzoic acid was prepared following General Experimental Procedure 8. tert-Butyl 5-methoxy-4-((7-(4-(methoxycarbonyl)phenyl)-4-methyl-5-(trifluoromethyl)-1,4-diazepan-1-yl)methyl)-7-methyl-1H-indole-1-carboxylate (60 mg, 1.0 eq, 0.10 mmol) gave 4-(4-((5-methoxy-7-methyl-1H-indol-4-yl)methyl)-1-methyl-7-(trifluoromethyl)-1,4-diazepan-5-yl)benzoic acid (27.3 mg, 56%) product as a white solid. LCMS (31697 LCMS-5 C3) RT: 2.020 min and 2.083 min (diastereomeric ratio 1/1); area % (215 nm): 98.3%; m/z=476.2 [M+H]⁺.

### Example 111. 4-(5-((5-Methoxy-7-methyl-1H-indol-4-yl)methyl)-4,5,6,7-tetrahydropyrazolo[1,5-a]pyrazin-6-yl)benzoic acid

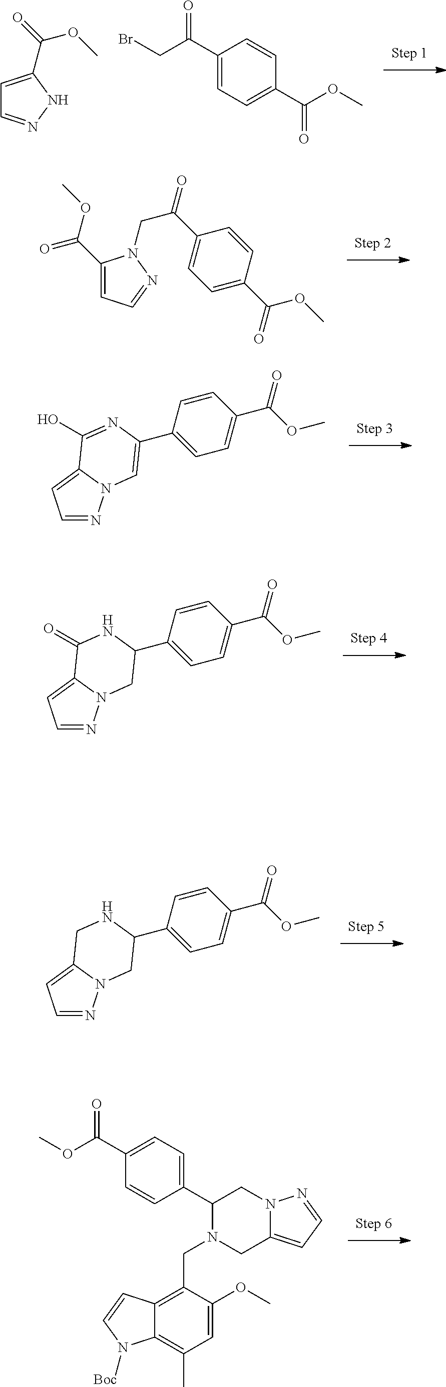

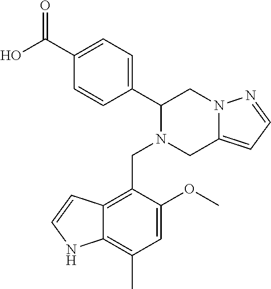 [SMILES: COc1cc(C)c2[nH]ccc2c1CN1Cc2ccnn2CC1c1ccc(C(=O)O)cc1]

Step 1. Methyl 1-(2-(4-(methoxycarbonyl)phenyl)-2-oxoethyl)-1H-pyrazole-5-carboxylate was prepared with the following procedure. To a solution of methyl 1H-pyrazole-5-carboxylate (1.2 g, 1.0 eq, 9.5 mmol) and methyl 4-(bromoacetyl)benzoate (2.63 g, 1.1 eq, 10.2 mmol) in anhydrous CH₃CN (50 mL) was added K₂CO₃ (1.4 g, 1.1 eq, 10 mmol) The reaction mixture was stirred at 50° C. for 16 hours. The reaction was worked up; diluted with water (100 mL) and removed CH₃CN was removed under reduced pressure. The residue was extracted with ethyl acetate (3×30 mL). The pooled organic layers were washed with brine, dried over Na₂SO₄, filtered, and concentrated under reduced pressure. The residue was purified by automated normal phase FCC to afford methyl 1-(2-(4-(methoxycarbonyl)phenyl)-2-oxoethyl)-1H-pyrazole-5-carboxylate (292 mg, 10%) as an orange solid. ¹H-NMR (400 MHz, CDCl₃) δ 8.15 (d, 2H), 8.02 (d, 2H), 7.60 (d, J=2.0 Hz, 1H), 6.94 (d, J=2.0 Hz, 1H), 6.02 (s, 2H), 3.95 (s, 3H), 3.80 (s, 3H).

Step 2. Methyl 4-(4-hydroxypyrazolo[1,5-a]pyrazin-6-yl)benzoate was prepared with the following procedure. To a solution of methyl 1-(2-(4-(methoxycarbonyl)phenyl)-2-oxoethyl)-1H-pyrazole-5-carboxylate (292 mg, 1.0 eq, 966 μmol) in AcOH (16 mL) was added NH₄OAc (755 mg, 10.1 Eq, 9.80 mmol). The reaction mixture was stirred at 125° C. for 16 hours. The reaction was worked up; diluted with water (10 mL). and filtered. The residue was washed with DCM/MeOH (1:1). The remaining solids were dried under reduced pressure to afford methyl 4-(4-hydroxypyrazolo[1,5-a]pyrazin-6-yl)benzoate (165 mg, 63.4%) as a green solid. ¹H-NMR (400 MHz, DMSO) δ 11.68 (s, 1H), 8.24 (s, 1H), 8.02 (d, 2H), 7.98 (d, J=2.2 Hz, 1H), 7.92 (d, 2H), 7.07 (d, J=2.2 Hz, 1H), 3.88 (s, 3H).

Step 3. Methyl 4-(4-oxo-4,5,6,7-tetrahydropyrazolo[1,5-a]pyrazin-6-yl)benzoate was prepared with the following procedure. To a solution of methyl 4-(4-hydroxypyrazolo[1,5-a]pyrazin-6-yl)benzoate (165 mg, 1.0 eq, 613 μmol) in 1,4-dioxane (7 mL) was added 10% Pd/C (32.6 mg, 0.05 Eq, 30.6 μmol). The flask was purged with H₂ before heated under an H₂ atmosphere (balloon) at 100° C. for 15 h. The reaction was worked up; filtered through over Celite and the filtrate concentrated to afford methyl 4-(4-oxo-4,5,6,7-tetrahydropyrazolo[1,5-a]pyrazin-6-yl)benzoate (154 mg, 92.6%) as a white solid. ¹H-NMR (400 MHz, CDCl₃) δ 8.09 (d, 2H), 7.56 (d, J=2.0 Hz, 1H), 7.47 (d, 2H), 6.93 (d, J=2.1 Hz, 1H), 5.99 (s, 1H), 5.18-5.11 (m, 1H), 4.60 (dd, J=13.1, 4.8, 1.0 Hz, 1H), 4.34 (dd, J=13.1, 9.9 Hz, 1H), 3.92 (s, 3H).

Step 4. Methyl 4-(4,5,6,7-tetrahydropyrazolo[1,5-a]pyrazin-6-yl)benzoate was prepared following General Experimental Procedure 10. Methyl 4-(4-oxo-4,5,6,7-tetrahydropyrazolo[1,5-a]pyrazin-6-yl)benzoate (154 mg, 1.0 eq, 568 μmol) gave crude methyl 4-(4,5,6,7-tetrahydropyrazolo[1,5-a]pyrazin-6-yl)benzoate (211 mg). LCMS (General 3 basic) RT: 0.98 min; area % (254 nm): 68.6%; m/z=258.2 [M+H]⁺.

Step 5. tert-Butyl 5-methoxy-4-((6-(4-(methoxycarbonyl)phenyl)-6,7-dihydropyrazolo[1,5-a]pyrazin-5 (4H)-yl)methyl)-7-methyl-1H-indole-1-carboxylate was prepared following General Experimental Procedure 4. Methyl 4-(4,5,6,7-tetrahydropyrazolo[1,5-a]pyrazin-6-yl)benzoate (146 mg, 1.0 eq, 568 μmol) and tert-butyl 4-formyl-5-methoxy-7-methyl-1H-indole-1-carboxylate (247 mg, 1.5 eq, 852 μmol) gave tert-butyl 5-methoxy-4-((6-(4-(methoxycarbonyl)phenyl)-6,7-dihydropyrazolo[1,5-a]pyrazin-5 (4H)-yl)methyl)-7-methyl-1H-indole-1-carboxylate (70 mg, 23%). LCMS (General 3 acidic) RT: 1.28 min; area % (254 nm): 83.9%; m/z=531.31 [M+H]⁺.

Step 6. 4-(5-((5-Methoxy-7-methyl-1H-indol-4-yl)methyl)-4,5,6,7-tetrahydropyrazolo[1,5-a]pyrazin-6-yl)benzoic acid was prepared following General Experimental Procedure 8. tert-Butyl 5-methoxy-4-((6-(4-(methoxycarbonyl)phenyl)-6,7-dihydropyrazolo[1,5-a]pyrazin-5 (4/I)-yl)methyl)-7-methyl-1H-indole-1-carboxylate (15 mg, 1.0 eq, 28 μmol) gave 4-(5-((5-methoxy-7-methyl-1H-indol-4-yl)methyl)-4,5,6,7-tetrahydropyrazolo[1,5-a]pyrazin-6-yl)benzoic acid (5.81 mg, 49%) as a white solid. LCMS (31697 LCMS-5 C3) RT: 1.871 min; area % (215 nm): 97.8%; m/z=417.2 [M+H]⁺. ¹H-NMR (400 MHz, CD₃OD) δ 8.10 (d, 2H), 7.67 (d, 2H), 7.43 (d, J=2.0 Hz, 1H), 7.23 (d, J=3.2 Hz, 1H), 6.73 (s, 1H), 6.48 (d, J=3.1 Hz, 1H), 5.95 (d, J=2.0 Hz, 1H), 4.44-4.37 (m, 1H), 4.35-4.28 (m, 1H), 4.21 (dd, J=8.8, 4.6 Hz, 1H), 3.93-3.84 (m, 2H), 3.80 (s, 3H), 3.68-3.57 (m, 2H), 2.49 (s, 3H).

### Example 112. 4-(8-((5-Methoxy-7-methyl-1H-indol-4-yl)methyl)octahydropyrazino[2,1-c][1,4]oxazin-7-yl)benzoic acid

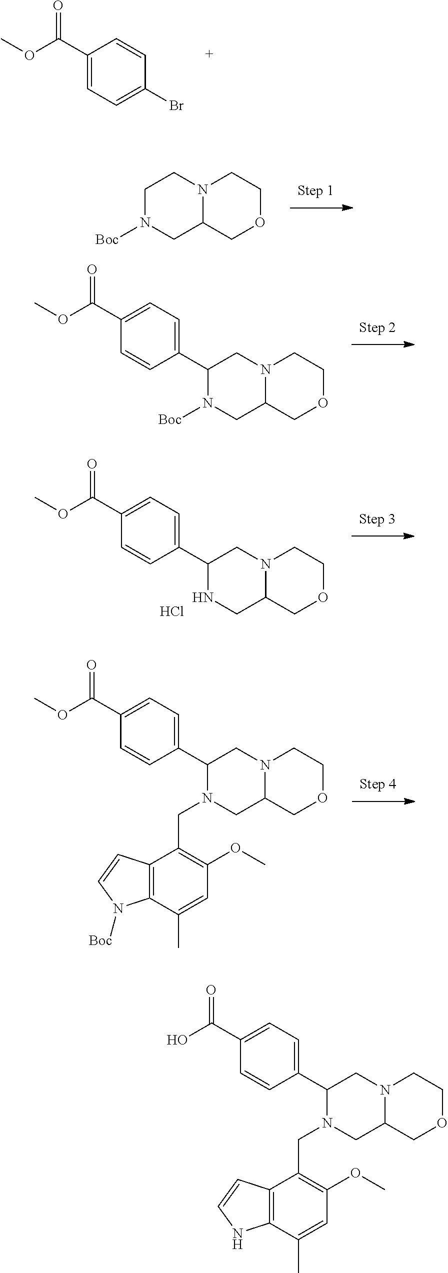

Step 1. tert-Butyl 7-(4-(methoxycarbonyl)phenyl)hexahydropyrazino[2,1-c][1,4]oxazine-8 (1H)-carboxylate was prepared following a procedure described above. tert-Butyl hexahydropyrazino[2,1-c][1,4]oxazine-8 (1H)-carboxylate (500 mg, 2.06 mmol), TMEDA (0.32 mL, 2.17 mmol), 1.6 M sec-butyllithium in cyclohexan/hexane (1.67 mL, 2.17 mmol), 1 M ZnCl₂ in THF (3.8 mL, 2.68 mmol), methyl 4-bromobenzoate (444 mg, 2.06 mmol), tri-tert-butylphosphoniumtetrafluoroborate (48 mg, 165 μmol) and PdOAc₂ (18.5 mg, 83 μmol) gave tert-butyl 7-(4-(methoxycarbonyl)phenyl)hexahydropyrazino[2,1-c][1,4]oxazine-8 (1H)-carboxylate (126 mg, 16%). ¹H-NMR (400 MHz, CDCl₃) δ 8.01-7.93 (m, 2H), 7.45-7.37 (m, 2H), 3.90 (M, 4H), 3.72-3.48 (m, 6H), 3.42-3.19 (m, 1H), 3.06 (s, 2H), 2.53 (d, J=10.5 Hz, 2H), 1.42 (d, J=9.4 Hz, 9H).

Step 2. Methyl 4-(octahydropyrazino[2,1-c][1,4]oxazin-7-yl)benzoate hydrochloride was prepared according to the following procedure. To a solution of tert-butyl 7-(4-(methoxycarbonyl)phenyl)hexahydropyrazino[2,1-c][1,4]oxazine-8 (1H)-carboxylate (123 mg, 1.0 eq, 327 μmol) in DCM (10 mL) was added 4 N HCl in dioxane (0.4 mL, 5.0 eq, 1.63 mmol). The mixture was stirred at 20° C. After stirring for one week, more 4 N HCl in dioxane (0.4 mL, 5.0 eq, 1.63 mmol) was added. After two more days, the reaction mixture was concentrated under reduced pressure to afford crude methyl 4-(octahydropyrazino[2,1-c][1,4]oxazin-7-yl)benzoate hydrochloride (111 mg, 100%). LCMS (LCMS-24 Libraries) RT: 1.053 min; area % (265 nm): 96%; m/z=277.1 [M+H]⁺.

Step 3. tert-Butyl 5-methoxy-4-((7-(4-(methoxycarbonyl)phenyl)hexahydropyrazino[2,1-c][1,4]oxazin-8 (1H)-yl)methyl)-7-methyl-1H-indole-1-carboxylate was prepared according to the following procedure. To a suspension of methyl 4-(octahydropyrazino[2,1-c][1,4]oxazin-7-yl)benzoate hydrochloride (102 mg, 1.0 eq, 327 μmol) in THF (10 mL) were added Et₃N (0.6 mL, 1.5 eq, 490 μmol) and tert-butyl 4-formyl-5-methoxy-7-methyl-1H-indole-1-carboxylate (284 mg, 3.0 eq, 981 μmol) followed by sodium triacetoxyborohydride (277 mg, 4.0 eq, 1.31 mmol). The mixture was stirred at 20° C. for 16 h. The reaction was worked-up; added H₂O (10 mL) and extracted with EtOAc (2×20 mL). The organic layers were combined, washed with brine, dried over Na₂SO₄, filtered and then concentrated under reduced pressure to afford crude tert-butyl 5-methoxy-4-((7-(4-(methoxycarbonyl)phenyl)hexahydropyrazino[2,1-c][1,4]oxazin-8 (1H)-yl)methyl)-7-methyl-1H-indole-1-carboxylate (390 mg). LCMS (LCMS-24 Libraries) RT: 2.362 min; area % (265 nm): 35%; m/z=550.4 [M+H]⁺.

Step 4. 4-(8-((5-methoxy-7-methyl-1H-indol-4-yl)methyl)octahydropyrazino[2,1-c][1,4]oxazin-7-yl)benzoic acid was prepared following General Experimental Procedure 8. Crude tert-butyl 5-methoxy-4-((7-(4-(methoxycarbonyl)phenyl)hexahydropyrazino[2,1-c][1,4]oxazin-8 (1H)-yl)methyl)-7-methyl-1H-indole-1-carboxylate (390 mg) gave 4-(8-((5-methoxy-7-methyl-1H-indol-4-yl)methyl)octahydropyrazino[2,1-c][1,4]oxazin-7-yl)benzoic acid (65 mg, 46%). LCMS (31697 LCMS-5 C3) RT: 1.656 min; area % (215 nm): 98%; m/z=436.2 [M+H]⁺. ¹H-NMR (400 MHz, CD₃OD) δ 7.89 (d, 2H), 7.73 (d, J=7.9 Hz, 2H), 7.28 (d, J=3.1 Hz, 1H), 6.73 (s, 1H), 6.54 (d, J=3.1 Hz, 1H), 4.10-4.02 (m, 1H), 3.89 (s, 2H), 3.83 (s, 3H), 3.68 (td, J=11.2, 2.4 Hz, 1H), 3.56 (dd, J=11.4, 3.4 Hz, 2H), 3.51-3.44 (m, 1H), 3.04-2.89 (m, 3H), 2.83 (dd, J=11.4, 3.4 Hz, 1H), 2.61 (d, J=12.6 Hz, 1H), 2.48 (s, 3H), 2.43-2.22 (m, 2H).

### Example 113. Enantiomer 1 of 4-(4-acetyl-1-((5-methoxy-7-methyl-1H-indol-4-yl)methyl)-1,4-diazepan-2-yl)benzoic acid

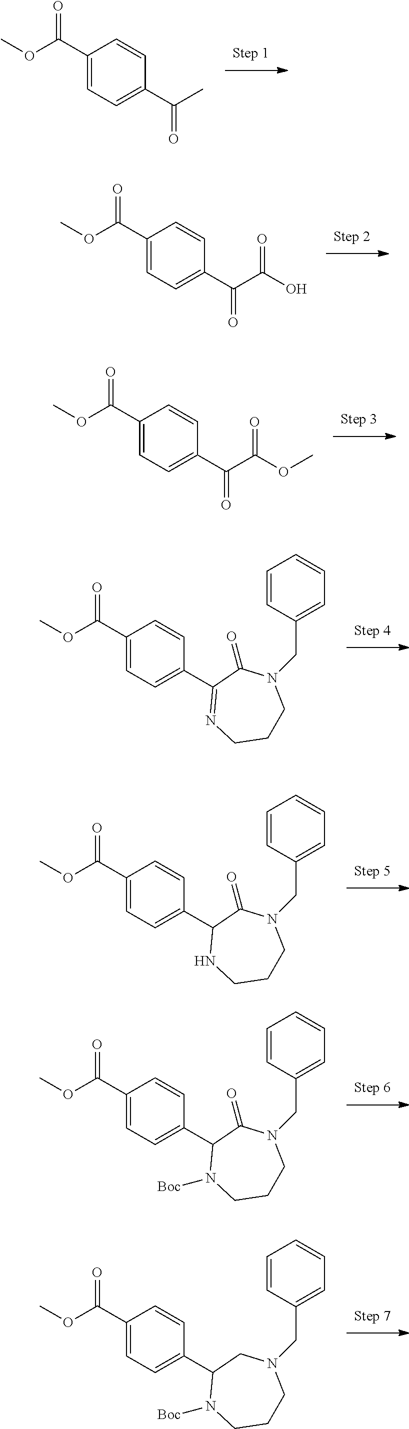

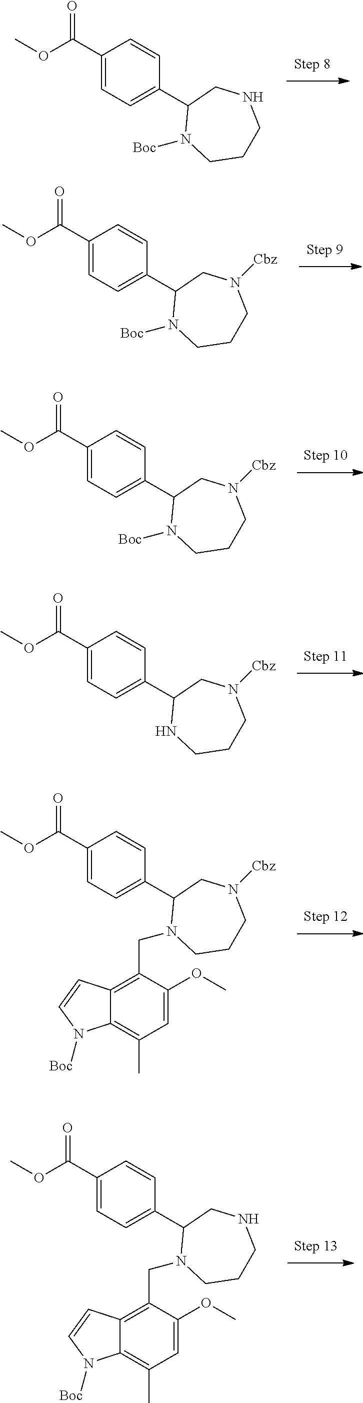 [SMILES: COC(=O)c1ccc(C2CNCCCN2C(=O)OC(C)(C)C)cc1 ; COC(=O)c1ccc(C2CN(C(=O)OCc3ccccc3)CCCN2C(=O)OC(C)(C)C)cc1 ; COC(=O)c1ccc(C2CN(C(=O)OCc3ccccc3)CCCN2C(=O)OC(C)(C)C)cc1 ; COC(=O)c1ccc(C2CN(C(=O)OCc3ccccc3)CCCN2)cc1 ; COC(=O)c1ccc(C2CN(C(=O)OCc3ccccc3)CCCN2Cc2c(OC)cc(C)c3c2ccn3C(=O)OC(C)(C)C)cc1 ; COC(=O)c1ccc(C2CNCCCN2Cc2c(OC)cc(C)c3c2ccn3C(=O)OC(C)(C)C)cc1]

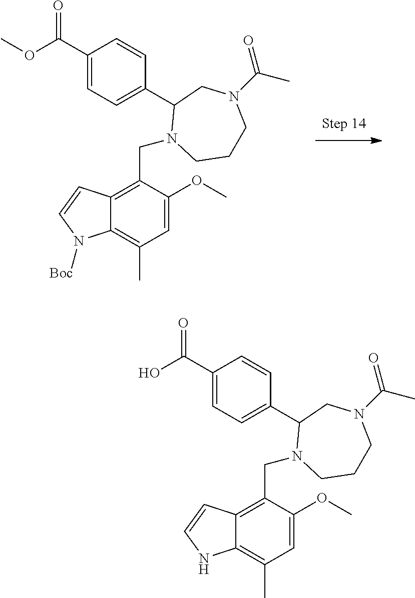 [SMILES: COC(=O)c1ccc(C2CN(C(C)=O)CCCN2Cc2c(OC)cc(C)c3c2ccn3C(=O)OC(C)(C)C)cc1 ; COc1cc(C)c2[nH]ccc2c1CN1CCCN(C(C)=O)CC1c1ccc(C(=O)O)cc1]

Step 1. 2-(4-(Methoxycarbonyl)phenyl)-2-oxoacetic acid was prepared with the following procedure. To a solution of methyl 4-acetylbenzoate (98.6 g, 1.0 eq, 553 mmol) in pyridine (1.0 L) was added selenium dioxide (123 g, 2.0 eq, 1.11 mol) and the resulting mixture was heated to 120° C. for 15 h. The reaction was worked up; filtered over Celite and washed with MeOH. The filtrate was concentrated under reduced pressure to afford 2-(4-(methoxycarbonyl)phenyl)-2-oxoacetic acid as orange solid (189 g, quant.). LCMS (LCMS-4 General 3 AB) RT: 0.779 min; area % (254 nm): 95.4%; mass not observed.

Step 2. Methyl 4-(2-methoxy-2-oxoacetyl)benzoate was prepared with the following procedure. To a solution of 2-(4-(methoxycarbonyl)phenyl)-2-oxoacetic acid (180.0 g, 1.0 eq, 864.7 mmol) in MeOH (1.0 L) was carefully added 98% H₂SO₄ (101.4 mL, 2.2 eq, 1.902 mol) and the mixture was heated at 75° C. for 15 h. The reaction was worked-up; filtered and the residue was washed with EtOAc. The filtrate was dried over Na₂SO₄, filtered and concentrated under reduced pressure to afford methyl 4-(2-methoxy-2-oxoacetyl)benzoate as a pink solid (117.1 g, 61%). LCMS (General 3 Acidic) RT: 1.05 min; area % (254 nm): 96.0%; mass not observed.

Step 3. Methyl 4-(1-benzyl-2-oxo-2,5,6,7-tetrahydro-1H-1,4-diazepin-3-yl)benzoate was prepared with the following procedure. To a ssupension of methyl 4-(2-methoxy-2-oxoacetyl)benzoate (111.74 g, 1.0 eq, 301.73 mmol) in toluene (1 L) were added N1-benzylpropane-1,3-diamine (49.559 g, 1.0 eq, 301.73 mmol) and AcOH (19.00 mL, 1.1 eq, 331.90 mmol). The resulting mixture was heated at 130° C. for 3 h. The reaction was worked up; concentrated under reduced pressure and the residue was dissolved in DCM. The organic layer was washed with sat. aq. NaHCO₃ (twice), H₂O, dried over Na₂SO₄, and concentrated under reduced pressure. The residue was purified by automated normal phase FCC to afford methyl 4-(1-benzyl-2-oxo-2,5,6,7-tetrahydro-1H-1,4-diazepin-3-yl)benzoate (90.8 g, 69%) as a yellow solid. LCMS (General 3 Basic) RT: 1.53 min; area % (254 nm): 80.9%; m/z=337.2 [M+H]⁺.

Step 4. Methyl 4-(4-benzyl-3-oxo-1,4-diazepan-2-yl)benzoate was prepared with the following procedure. To a solution of methyl 4-(1-benzyl-2-oxo-2,5,6,7-tetrahydro-1H-1,4-diazepin-3-yl)benzoate (90 g, 1.0 eq, 0.27 mol) in EtOAc (2.5 L) was added a suspension of 10% Pd/C (28 g, 0.1 eq, 27 mmol) in EtOAc (1 mL). The reaction mixture was purged with H₂ and stirred under H₂ atmosphere (balloon) for 15 h. The reaction was worked up; filtered over Celite and the filtrate was concentrated under reduced pressure to afford methyl 4-(4-benzyl-3-oxo-1,4-diazepan-2-yl)benzoate (91.2 g, 100%) as a yellow oil. LCMS (General 3 Basic) RT: 1.32 min; area % (254 nm): 67.8%; m/z=339.2 [M+H]⁺.

Step 5. tert-Butyl 4-benzyl-2-(4-(methoxycarbonyl)phenyl)-3-oxo-1,4-diazepane-1-carboxylate was prepared with the following procedure. To a solution of methyl 4-(4-benzyl-3-oxo-1,4-diazepan-2-yl)benzoate (91.2 g, 1.0 eq, 208 mmol) in DCM (1.5 L) were added DIPEA (80.5 g, 108 mL, 3.0 eq, 623 mmol) and Boc₂O (67.9 g, 1.5 eq, 311 mmol). The resulting mixture was allowed to stir at 20° C. for 15 h. The reaction was worked up; partially concentrated under reduced pressure and the organic layer was washed with 5% HCl, H₂O/Brine 1:1, dried over Na₂SO₄, filtered, and concentrated under reduced pressure. The residue was purified by automated normal phase FCC to afford tert-butyl 4-benzyl-2-(4-(methoxycarbonyl)phenyl)-3-oxo-1,4-diazepane-1-carboxylate as a yellow oil (81.3 g, 89%). LCMS (General 3 Basic) RT: 1.76 min; area % (254 nm): 97.1%; m/z=383.3 [M−Boc]⁺.

Step 6. tert-Butyl 4-benzyl-2-(4-(methoxycarbonyl)phenyl)-1,4-diazepane-1-carboxylate was prepared with the following procedure. A solution of tert-butyl 4-benzyl-2-(4-(methoxycarbonyl)phenyl)-3-oxo-1,4-diazepane-1-carboxylate (7.988 g, 1.0 eq, 18.22 mmol) in dry THF (100 mL) was bubbled with N₂. Acetylacetonato (1,5-cyclooctadiene) rhodium (423.8 mg, 0.075 eq, 1.366 mmol) and silylbenzene (9.857 g, 11.3 mL, 5.0 eq, 91.08 mmol) were added at 0° C. and the resulting mixture was heated to reflux and allowed to stir at this temperature for 30 h. The reaction was worked up; concentrated under reduced pressure and the residue purified by automated normal phase FCC to afford tert-butyl 4-benzyl-2-(4-(methoxycarbonyl)phenyl)-1,4-diazepane-1-carboxylate. (4.18 g, 54%). LCMS (General 3 Basic) RT: 2.02 min; area % (254 nm): 99.3%; m/z=425.5 [M+H]⁺.

Step 7. tert-Butyl 2-(4-(methoxycarbonyl)phenyl)-1,4-diazepane-1-carboxylate was prepared with the following procedure. To a solution of tert-butyl 4-benzyl-2-(4-(methoxycarbonyl)phenyl)-1,4-diazepane-1-carboxylate (4.18 g, 1.0 eq, 9.85 mmol) in EtOAc (100 mL) was added 10% Pd/C (1.05 g, 0.10 eq, 985 μmol). The resulting mixture was allowed to stir under a H₂ atmosphere (balloon) at 20° C. for 72 h. The reaction was worked up; filtered over Celite and the filtrate was concentrated under reduced pressure to afford tert-butyl 2-(4-(methoxycarbonyl)phenyl)-1,4-diazepane-1-carboxylate (2.85 g, 87%) as yellow oil. LCMS (LCMS-24 Libraries) RT: 1.524 min; area % (265 nm): 77.0%; m/z=335.2 [M+H]⁺.

Step 8. 4-Benzyl 1-(tert-butyl) 2-(4-(methoxycarbonyl)phenyl)-1,4-diazepane-1,4-dicarboxylate was prepared with the following procedure. To a solution of tert-butyl 2-(4-(methoxycarbonyl)phenyl)-1,4-diazepane-1-carboxylate (1.02 g, 1.0 eq, 3.05 mmol) in DCM (20 mL) were added DIPEA (584 μL, 1.1 eq, 3.36 mmol) and Cbz-Cl (1.04 g, 871 μL, 2 eq, 6.10 mmol). The resulting mixture was allowed to stir at 20° C. for 15 h. The reaction was worked up; concentrated under reduced pressure and purified by automated normal phase FCC to afford 4-benzyl 1-(tert-butyl) 2-(4-(methoxycarbonyl)phenyl)-1,4-diazepane-1,4-dicarboxylate (1.43 g, 100%). LCMS (LCMS-24 Libraries) RT: 2.015 min; area % (265 nm): 79.7%; m/z=369.1 [M−Boc]⁺.

Step 9. Enantiomer 1 of 4-benzyl 1-(tert-butyl) 2-(4-(methoxycarbonyl)phenyl)-1,4-diazepane-1,4-dicarboxylate was prepared according to the following procedure. Racemic 4-benzyl 1-(tert-butyl) 2-(4-(methoxycarbonyl)phenyl)-1,4-diazepane-1,4-dicarboxylate was separated by chiral SFC (100 ml 20% A) to obtain enantiomer 1 of 4-benzyl 1-(tert-butyl) 2-(4-(methoxycarbonyl)phenyl)-1,4-diazepane-1,4-dicarboxylate (356 mg). First eluting enantiomer (31697AD UPC² 5m Cel3 M); RT=2.167 min; area % (224 nm); 100%; m/z=369.1 [(M−Boc)+H]⁺

Step 10. Enantiomer 1 of benzyl 3-(4-(methoxycarbonyl)phenyl)-1,4-diazepane-1-carboxylate hydrochloride was prepared with the following procedure. To a solution of enantiomer 1 of 4-benzyl 1-(tert-butyl) 2-(4-(methoxycarbonyl)phenyl)-1,4-diazepane-1,4-dicarboxylate (356 mg, 1.0 eq, 760 μmol) in DCM (10 mL) was added 4 N HCl in dioxane (1.90 mL, 10 eq, 7.60 mmol). The resulting mixture was allowed to stir at 20° C. for 15 h. The reaction was worked-up; concentrated under reduced pressure to afford crude enantiomer 1 of benzyl 3-(4-(methoxycarbonyl)phenyl)-1,4-diazepane-1-carboxylate hydrochloride (535 mg, quant.). LCMS (LCMS-24 Libraries) RT: 1.692 min; area % (265 nm): 96.4%; m/z=369.2 [M+H]⁺.

Step 11. Enantiomer 1 of tert-butyl 4-((4-((benzyloxy)carbonyl)-2-(4-(methoxycarbonyl)phenyl)-1,4-diazepan-1-yl)methyl)-5-methoxy-7-methyl-1H-indole-1-carboxylate carboxylate was prepared with the following procedure. To a solution of enantiomer 1 of benzyl 3-(4-(methoxycarbonyl)phenyl)-1,4-diazepane-1-carboxylate hydrochloride (280 mg, 1.0 eq, 760 μmol) in DCE (10 mL) was added Et₃N (530 μL, 5.0 eq, 3.80 mmol). The mixture was allowed to stir at 20° C. for 5 minutes before adding AcOH (500 μL, 11.5 eq, 8.73 mmol) (pH=5) and tert-butyl 4-formyl-5-methoxy-7-methyl-1H-indole-1-carboxylate (286 mg, 1.3 eq, 988 μmol). The mixture was allowed to stir at 20° C. for 10 minutes before adding sodium triacetoxyborohydride (644 mg, 4.0 eq, 3.04 mmol). The mixture was allowed to stir at 20° C. for 15 h. The reaction was worked up; quenched with sat. aq. NaHCO₃ and extracted with DCM. The pooled organic layers were dried over Na₂SO₄, filtered, and concentrated under reduced pressure. The residue was purified by automated normal phase FCC to afford enantiomer 1 of tert-butyl 4-((4-((benzyloxy)carbonyl)-2-(4-(methoxycarbonyl)phenyl)-1,4-diazepan-1-yl)methyl)-5-methoxy-7-methyl-1H-indole-1-carboxylate carboxylate (454 mg, 93%). LCMS (LCMS-24 Libraries) RT: 2.518 min; area % (265 nm): 93.5%; m/z=642.4 [M+H]⁺.

Step 12. Enantiomer 1 of tert-butyl 5-methoxy-4-((2-(4-(methoxycarbonyl)phenyl)-1,4-diazepan-1-yl)methyl)-7-methyl-1H-indole-1-carboxylate was prepared with the following procedure. To a solution of enantiomer 1 of tert-butyl 4-((4-((benzyloxy)carbonyl)-2-(4-(methoxycarbonyl)phenyl)-1,4-diazepan-1-yl)methyl)-5-methoxy-7-methyl-1H-indole-1-carboxylate (454 mg, 1.0 eq, 707 μmol) in EtOAc (10 mL) was added 10% Pd/C (75.3 mg, 0.10 eq, 70.7 μmol) was added and the resulting mixture was allowed to stir under a H₂ atmosphere (balloon) at 20° C. for 15 h. The reaction was worked up; filtered over Celite and the filtrate was concentrated under reduced pressure to afford enantiomer 1 of tert-butyl 5-methoxy-4-((2-(4-(methoxycarbonyl)phenyl)-1,4-diazepan-1-yl)methyl)-7-methyl-1H-indole-1-carboxylate (163 mg, 45.4%). LCMS (LCMS-24 Libraries) RT: 1.740 min; area % (215 nm): 85.8%; mass not observed.

Step 13. Enantiomer 1 of tert-butyl 4-((4-acetyl-2-(4-(methoxycarbonyl)phenyl)-1,4-diazepan-1-yl)methyl)-5-methoxy-7-methyl-1H-indole-1-carboxylate was prepared with the following procedure. To a solution of enantiomer 1 of tert-butyl-5-methoxy-4-((2-(4-(methoxycarbonyl)phenyl)-1,4-diazepan-1-yl)methyl)-7-methyl-1H-indole-1-carboxylate (82 mg, 1.0 eq, 0.16 mmol) in DCM (10 mL) was added acetic anhydride (17 μL, 1.1 eq, 0.18 mmol). The resulting mixture was allowed to stir at 20° C. for 15 h. The reaction was worked up; concentrated under reduced pressure and the residue was purified by automated normal phase FCC to afford enantiomer 1 of tert-butyl 4-((4-acetyl-2-(4-(methoxycarbonyl)phenyl)-1,4-diazepan-1-yl)methyl)-5-methoxy-7-methyl-1H-indole-1-carboxylate (62 mg, 70%). LCMS (LCMS-24 Libraries) RT: 2.205 min; area % (265 nm): 95.9%; m/z=550.3 [M+H]⁺.

Step 14. Enantiomer 1 of 4-(4-acetyl-1-((5-methoxy-7-methyl-1H-indol-4-yl)methyl)-1,4-diazepan-2-yl)benzoic acid was prepared following General Experimental Procedure 8. Enantiomer 1 of tert-butyl 4-((4-acetyl-2-(4-(methoxycarbonyl)phenyl)-1,4-diazepan-1-yl)methyl)-5-methoxy-7-methyl-1H-indole-1-carboxylate (62 mg) gave enantiomer 1 of 4-(4-acetyl-1-((5-methoxy-7-methyl-1H-indol-4-yl)methyl)-1,4-diazepan-2-yl)benzoic acid (5 mg, 10%) as a white solid. LCMS (31697 LCMS-5 C3) RT: 1.58 min; area % (215 nm): 93.1%; m/z=438.3 [M+3H]⁺. ¹H NMR (400 MHz, CD₃OD) δ 8.05 (t, J=7.3 Hz, 2H), 7.59 (dd, J=8.4, 2.2 Hz, 2H), 7.20 (dd, J=8.9, 3.2 Hz, 1H), 6.68 (d, J=7.2 Hz, 1H), 6.33 (dd, J=5.8, 3.2 Hz, 1H), 4.58 (s, 1H), 4.25 (d, J=14.6 Hz, 1H), 3.72 (s, 3H), 2.46 (d, J=4.4 Hz, 3H), 2.16 (s, 2H), 2.07 (s, 1H).

### Example 114. Enantiomer 2 of 4-(4-acetyl-1-((5-methoxy-7-methyl-1H-indol-4-yl)methyl)-1,4-diazepan-2-yl)benzoic acid

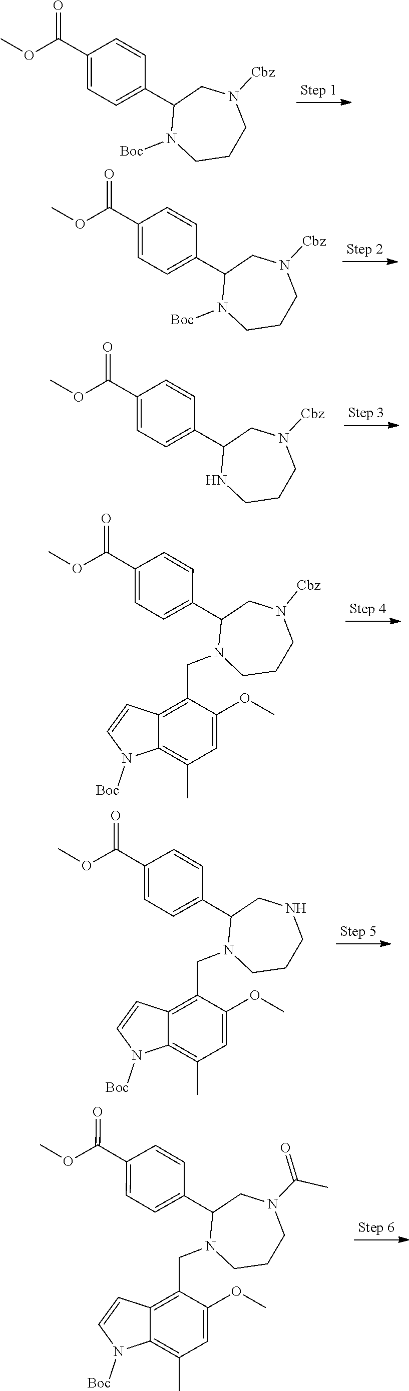

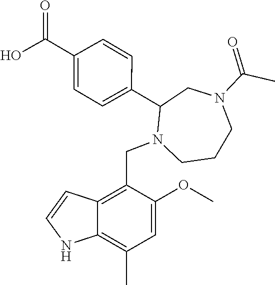 [SMILES: COc1cc(C)c2[nH]ccc2c1CN1CCCN(C(C)=O)CC1c1ccc(C(=O)O)cc1]

Step 1. Enantiomer 2 of 4-benzyl 1-(tert-butyl) 2-(4-(methoxycarbonyl)phenyl)-1,4-diazepane-1,4-dicarboxylate was prepared according to the following procedure. Racemic 4-benzyl 1-(tert-butyl) 2-(4-(methoxycarbonyl)phenyl)-1,4-diazepane-1,4-dicarboxylate was separated by chiral SFC (100 ml 20% A) to obtain enantiomer 2 of 4-benzyl 1-(tert-butyl) 2-(4-(methoxycarbonyl)phenyl)-1,4-diazepane-1,4-dicarboxylate (374 mg). Second eluting enantiomer (31697AD UPC² 5m Cel3 M); RT=2.543 min; area % (224 nm); 98.9%; m/z=369.1 [(M−Boc)+H]⁺.

Step 2. Enantiomer 2 of benzyl 3-(4-(methoxycarbonyl)phenyl)-1,4-diazepane-1-carboxylate was prepared with the following procedure. To a solution of enantiomer 2 of 4-benzyl 1-(tert-butyl) 2-(4-(methoxycarbonyl)phenyl)-1,4-diazepane-1,4-dicarboxylate (374 mg, 1.0 eq, 798 μmol) in DCM (10 mL) was added 4 N HCl in dioxane (2.00 mL, 10 eq, 7.98 mmol). The resulting mixture was allowed to stir at 20° C. for 15 h. The reaction was worked-up; concentrated under reduced pressure to afford crude enantiomer 2 of Benzyl 3-(4-(methoxycarbonyl)phenyl)-1,4-diazepane-1-carboxylate hydrochloride (336.5 mg, quant.). LCMS (LCMS-24 Libraries) RT: 1.687 min; area % (265 nm): 97.0%; m/z=369.2 [M+H]⁺.

Step 3. Enantiomer 2 of tert-butyl 4-((4-((benzyloxy)carbonyl)-2-(4-(methoxycarbonyl)phenyl)-1,4-diazepan-1-yl)methyl)-5-methoxy-7-methyl-1H-indole-1-carboxylate carboxylate was prepared with the following procedure. To a solution of crude enantiomer 2 of benzyl 3-(4-(methoxycarbonyl)phenyl)-1,4-diazepane-1-carboxylate hydrochloride (294 mg, 1.0 eq, 798 μmol) in DCE (10 mL) was added Et₃N (556 μL, 5.0 eq, 3.99 mmol). The mixture was allowed to stir at 20° C. for 5 minutes before adding AcOH (500 μL, 11.5 eq, 8.73 mmol) (pH=5) and tert-butyl 4-formyl-5-methoxy-7-methyl-1H-indole-1-carboxylate (300 mg, 1.3 eq, 1.04 mmol). The mixture was allowed to stir at 20° C. for 10 minutes before sodium triacetoxyborohydride (677 mg, 4 eq, 3.19 mmol) was added. The mixture was allowed to stir at 20° C. for 15 h. The reaction was worked up; quenched with sat. aq. NaHCO₃ and extracted with DCM. The pooled organic layers were dried over Na₂SO₄, filtered, and concentrated under reduced pressure. The residue was purified by automated normal phase FCC to afford enantiomer 2 of tert-butyl 4-((4-((benzyloxy)carbonyl)-2-(4-(methoxycarbonyl)phenyl)-1,4-diazepan-1-yl)methyl)-5-methoxy-7-methyl-1H-indole-1-carboxylate carboxylate (394 mg, 76.9%). LCMS (LCMS-24 Libraries) RT: 2.518 min; area % (265 nm): 97.0%; m/z=642.4 [M+H]⁺.

Step 4. Enantiomer 2 of tert-butyl 5-methoxy-4-((2-(4-(methoxycarbonyl)phenyl)-1,4-diazepan-1-yl)methyl)-7-methyl-1H-indole-1-carboxylate was prepared with the following procedure. To a solution of enantiomer 2 of tert-butyl 4-((4-((benzyloxy)carbonyl)-2-(4-(methoxycarbonyl)phenyl)-1,4-diazepan-1-yl)methyl)-5-methoxy-7-methyl-1H-indole-1-carboxylate (394 mg, 1.0 eq, 614 μmol)) in EtOAc (10 mL) was added 10% Pd/C (65.3 mg, 0.1 eq, 61.4 μmol) and the resulting mixture was allowed to stir under a H₂ atmosphere (balloon) at 20° C. for 15 h. The reaction was worked up; filtered over Celite and the filtrate was concentrated under reduced pressure to afford enantiomer 2 of tert-butyl 5-methoxy-4-((2-(4-(methoxycarbonyl)phenyl)-1,4-diazepan-1-yl)methyl)-7-methyl-1H-indole-1-carboxylate (253 mg, 81%). LCMS (LCMS-24 Libraries) RT: 1.740 min, mass not observed.

Step 5. Enantiomer 2 of tert-butyl 4-((4-acetyl-2-(4-(methoxycarbonyl)phenyl)-1,4-diazepan-1-yl)methyl)-5-methoxy-7-methyl-1H-indole-1-carboxylate was prepared with the following procedure. To a solution of enantiomer 2 of tert-butyl-5-methoxy-4-((2-(4-(methoxycarbonyl)phenyl)-1,4-diazepan-1-yl)methyl)-7-methyl-1H-indole-1-carboxylate (126 mg, 1.0 eq, 248 μmol) in DCM (10 mL) was added acetic anhydride (25.8 μL, 1.1 eq, 273 μmol). The resulting mixture was allowed to stir at 20° C. for 15 h. The reaction was worked up; concentrated under reduced pressure and the residue purified by automated normal phase FCC to afforded enantiomer 2 of tert-butyl 4-((4-acetyl-2-(4-(methoxycarbonyl)phenyl)-1,4-diazepan-1-yl)methyl)-5-methoxy-7-methyl-1H-indole-1-carboxylate (66 mg, 48%). LCMS (LCMS-24 Libraries) RT: 2.206 min; area % (265 nm): 96.3%; m/z=550.3 [M+H]⁺.

Step 6. Enantiomer 2 of 4-(4-acetyl-1-((5-methoxy-7-methyl-1H-indol-4-yl)methyl)-1,4-diazepan-2-yl)benzoic acid was prepared following General Experimental Procedure 8. Enantiomer 2 of tert-butyl 4-((4-acetyl-2-(4-(methoxycarbonyl)phenyl)-1,4-diazepan-1-yl)methyl)-5-methoxy-7-methyl-1H-indole-1-carboxylate (66 mg) gave enantiomer 2 of 4-(4-acetyl-1-((5-methoxy-7-methyl-1H-indol-4-yl)methyl)-1,4-diazepan-2-yl)benzoic acid (5 mg, 10%) as a white solid. LCMS (31697 LCMS-5 C3) RT: 1.58 min; area % (215 nm): 94.9%; m/z=436.2 [M+H]⁺. ¹H-NMR (400 MHz, CD₃OD) δ 8.05 (t, J=7.3 Hz, 2H), 7.59 (dd, J=8.4, 2.2 Hz, 2H), 7.20 (dd, J=8.9, 3.2 Hz, 1H), 6.68 (d, J=7.2 Hz, 1H), 6.33 (dd, J=5.8, 3.2 Hz, 1H), 4.58 (s, 1H), 4.25 (d, J=14.6 Hz, 1H), 3.72 (s, 3H), 2.46 (d, J=4.4 Hz, 3H), 2.16 (s, 2H), 2.07 (s, 1H).

### Example 115. Enantiomer 2 of 4-(1-((5-methoxy-7-methyl-1H-indol-4-yl)methyl)-4-(methylsulfonyl)-1,4-diazepan-2-yl)benzoic acid

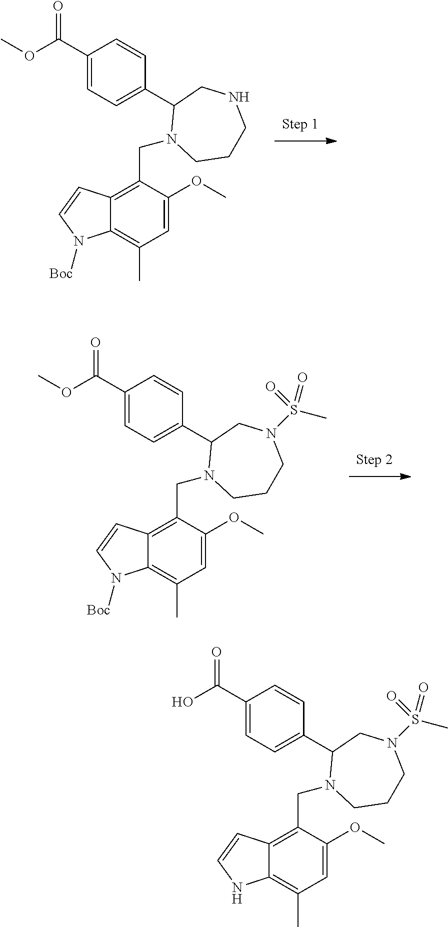

Step 1. Enantiomer 2 of tert-butyl 5-methoxy-4-((2-(4-(methoxycarbonyl)phenyl)-4-(methylsulfonyl)-1,4-diazepan-1-yl)methyl)-7-methyl-1H-indole-1-carboxylate was prepared with the following procedure. To a solution of enantiomer 2 of tert-butyl-5-methoxy-4-((2-(4-(methoxycarbonyl)phenyl)-1,4-diazepan-1-yl)methyl)-7-methyl-1H-indole-1-carboxylate (126 mg, 1.0 eq, 248 μmol) in DCM (10 mL) were added methanesulfonyl chloride (28.8 μL, 1.5 eq, 372 μmol) and Et₃N (38.1 μL, 1.1 eq, 273 μmol). The resulting mixture was allowed to stir at 20° C. for 15 h. The reaction was worked up; concentrated under reduced pressure and the residue was purified by automated normal phase FCC to afford enantiomer 2 of tert-butyl 5-methoxy-4-((2-(4-(methoxycarbonyl)phenyl)-4-(methylsulfonyl)-1,4-diazepan-1-yl)methyl)-7-methyl-1H-indole-1-carboxylate (40 mg, 28%). LCMS (LCMS-24 Libraries) RT: 2.259 min; area % (265 nm): 92.0%; m/z=586.3 [M+H]⁺.

Step 2. Enantiomer 2 of 4-(1-((5-methoxy-7-methyl-1H-indol-4-yl)methyl)-4-(methylsulfonyl)-1,4-diazepan-2-yl)benzoic acid was prepared following General Experimental Procedure 8. Enantiomer 2 of tert-butyl 5-methoxy-4-((2-(4-(methoxycarbonyl)phenyl)-4-(methylsulfonyl)-1,4-diazepan-1-yl)methyl)-7-methyl-1H-indole-1-carboxylate (40 mg) gave enantiomer 2 of 4-(1-((5-methoxy-7-methyl-1H-indol-4-yl)methyl)-4-(methylsulfonyl)-1,4-diazepan-2-yl)benzoic acid (5 mg, 20%) as a white solid. LCMS (31697 LCMS-5 C3) RT: 1.70 min; area % (215 nm): 96.7%; m/z=472.2 [M+H]⁺. ¹H NMR (400 MHz, CD₃OD) δ 7.98 (d, J=8.1 Hz, 2H), 7.86 (s, 1H), 7.52 (d, J=8.2 Hz, 2H), 7.14 (d, J=3.1 Hz, 1H), 6.65 (s, 1H), 6.35 (d, J=3.1 Hz, 1H), 4.56 (s, 1H), 3.79 (d, J=12.5 Hz, 2H), 3.75 (s, 3H), 3.70-3.59 (m, 3H), 3.55 (d, J=15.6 Hz, 2H), 3.36 (d, J=10.2 Hz, 3H), 3.19-3.07 (m, 3H), 2.80 (s, 3H), 2.71 (dd, J=14.1, 8.3 Hz, 2H), 2.45 (d, J=0.8 Hz, 3H), 2.01-1.87 (m, 2H), 1.87-1.79 (m, 1H), 1.29 (s, 2H).

### Example 116. Provided Compounds can Inhibit Complement Activation

Among other things, the present disclosure provides compounds and compositions that can modulate complement activation. In some embodiments, provided technologies inhibit complement activation. In some embodiments, provided technologies inhibit C3 convertase. Various technologies are available for assessing provided compounds, compositions and methods. In some embodiments, a useful assay is an assay described below. In some embodiments, IC50 of a provided compound measured in an assay is about or no more than about 1000, 900, 800, 700, 600, 500, 400, 300, 200, 100, 90, 80, 70, 60, 50, 40, 30, 20, or 10 nM. In some embodiments, a useful assay is a biochemical assay assessing inhibition of C3 convertase activity by a provided compound. In some embodiments, a purified, isolated or enriched C3 convertase is utilized. In some embodiments, CVF:Bb (e.g., assembled from three components Cobra Venom Factor (CVF), Complement Factor B (FB) and Complement Factor D (FD)) was utilized (certain results are presented as “A” in the Table below). In some embodiments, a compound was first contacted with CVF:Bb, followed by addition of C3 substrates. As those skilled in the art will appreciate, products C3a and/or C3b could be detected through a number of technologies (e.g., antibodies specific for a product) and reduction of levels of one or both of them could be utilized to assess inhibition. In some embodiments, a useful assay is Wieslab® assay (certain results are presented as “B” in the Table below).

In some embodiments, classical and alternative pathway activity are assessed using Wieslab® technologies. In some embodiments, Wieslab® assay were performed with a commercially available kit, using a protocol adapted for measurement of complement inhibitory activity of compounds. In some embodiments, human serum was utilized as a positive control. In some embodiments, human serum serves as a source of complement proteins, and inhibition of complement activity of a serum by various compounds were assessed. In some embodiments, a kit is COMPL AP330, Complement Alternative Pathway ELISA, Eagle Biosciences. In some embodiments, a kit is COMPL CP310, Complement Classical Pathway ELISA, Eagle Biosciences. In some embodiments, kits are available from VWR (102994-104, Complement Alternative pathway ELISA, Eagle Biosciences, Inc.; 102994-106, Complement Classical pathway ELISA, Eagle Biosciences, Inc.).

In some embodiments, a useful procedure described below is utilized.

1. Make fresh test compound stock at 1 mM.
2. Test compound serial dilution: dilute the stock to working stock concentrations. Add DMSO to dilution buffer using same dilution factor for the working stock so the composition of all the serial diluted stock is the same. In some embodiments, stock was serial diluted from the working stock 1:4 (for classical pathway) or 1:2 (for alternative pathway) using new diluted dilution buffers.
3. Dilute positive controls using the dilution buffer provided with the assay kit to 2× of the final conc., for classical pathway, 50.5×. Positive control is human serum, which contains complement proteins.
4. Add 125 μL of test compound solution and 125 μL of diluted positive control to a 96 well polypropylene plate, mix gently and cover the plate and incubate at 4° C. for 30 min.
5. Add 100 μL/well to the assay plate and incubate at 37° C. for 1 h. Typically, controls in the assay plate, for example, buffer alone for negative control, positive control alone to verify the complement activity of the reagent.
6. Wash plate and add 100 μL/well conjugate, incubate at room temperature for 30 min.
7. Wash plate and add 100 μL/well substrate, then incubate at room temperature for approximately 30 minutes. Periodically monitor the absorbance at 405 nm (OD405) in the positive control alone well.
8. Once the OD405 is >1, read the whole plate immediately. Or stop the reaction by adding 100 μL of 5 mM EDTA to each well and read within 60 minutes.

Certain results are provided below. A: C3 Convertase Assay IC₅₀ (nM). B: Wieslab AP IC50 (nM) (alternative pathway). NA: IC₅₀ greater than 10 μM. As demonstrated herein, various compounds can inhibit C3 convertase activity.

TABLE E-1
Certain compounds and assay data.

| Compound | A | B |
|---|---|---|
| 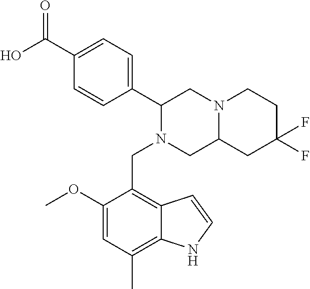 [SMILES: COc1cc(C)c2[nH]ccc2c1CN1CC2CC(F)(F)CCN2CC1c1ccc(C(=O)O)cc1] | 1409.555 | |
| 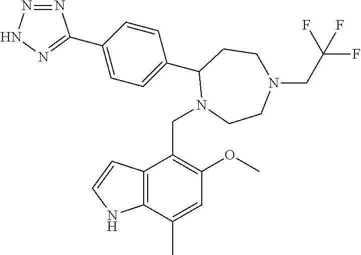 [SMILES: COc1cc(C)c2[nH]ccc2c1CN1CCN(CC(F)(F)F)CCC1c1ccc(-c2nn[nH]n2)cc1] | 770.141 | |
| 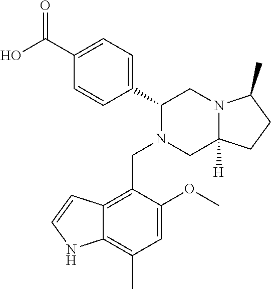 [SMILES: COc1cc(C)c2[nH]ccc2c1CN1C[C@@H]2CC[C@H](C)N2C[C@H]1c1ccc(C(=O)O)cc1] | 759.912 | |
| 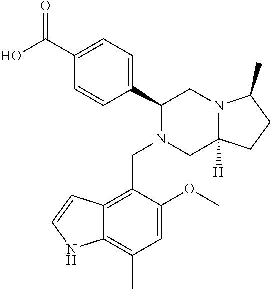 [SMILES: COc1cc(C)c2[nH]ccc2c1CN1C[C@@H]2CC[C@H](C)N2C[C@@H]1c1ccc(C(=O)O)cc1] | 12793.92 | |
| 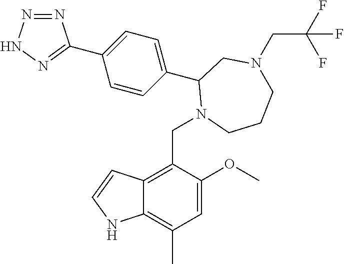 [SMILES: COc1cc(C)c2[nH]ccc2c1CN1CCCN(CC(F)(F)F)CC1c1ccc(-c2nn[nH]n2)cc1] | 175.597 | |
| 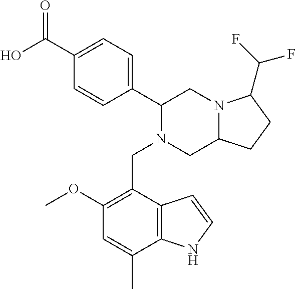 [SMILES: COc1cc(C)c2[nH]ccc2c1CN1CC2CCC(C(F)F)N2CC1c1ccc(C(=O)O)cc1] | 493.224 | |
| 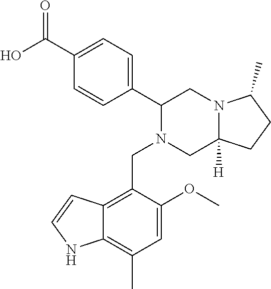 [SMILES: COc1cc(C)c2[nH]ccc2c1CN1C[C@@H]2CC[C@@H](C)N2CC1c1ccc(C(=O)O)cc1] | 2468.145 | |
| 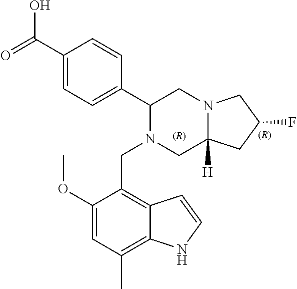 | 6965.563 | |
| 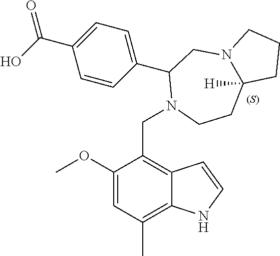 | 6975.81 | |
| 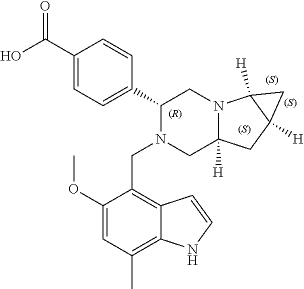 | 457.34 | |
| 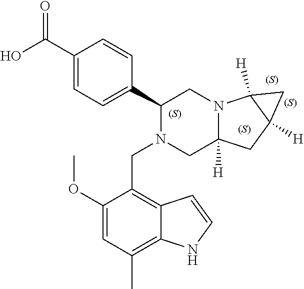 | 2079.036 | |
| 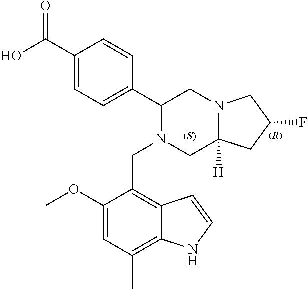 | 11669.76 | |
| 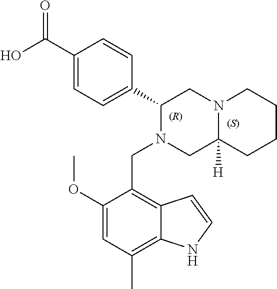 | 2606.864 | |
| 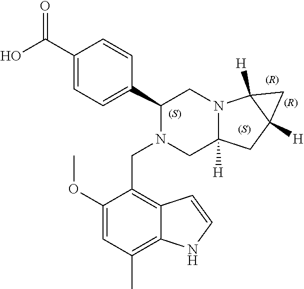 | 11977.98 | |
| 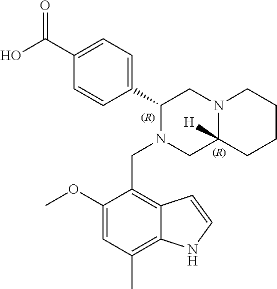 | 22.854 | |
| 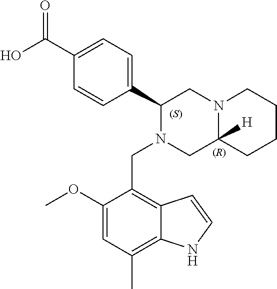 | 4186.848 | |
| 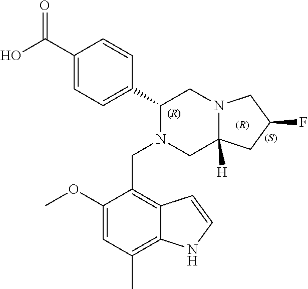 | 222.964 | |
| 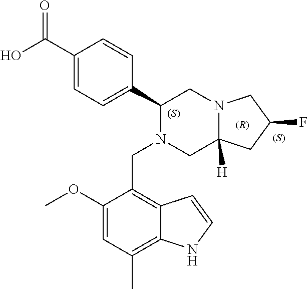 | 20673.95 | |
| 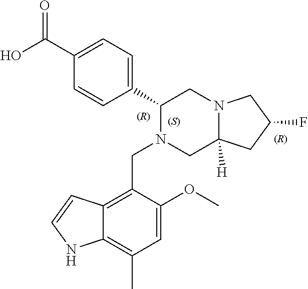 | 19024.53 | |
| 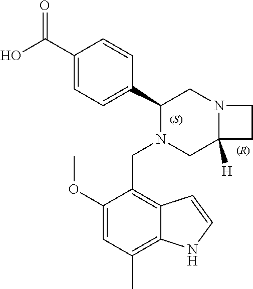 | 28144.27 | |
| 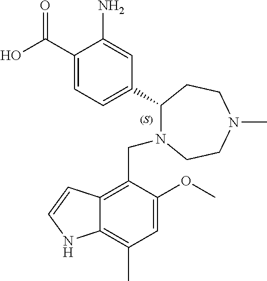 | 690.795 | |
| 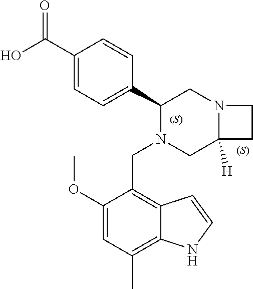 | 44160.48 | |
| 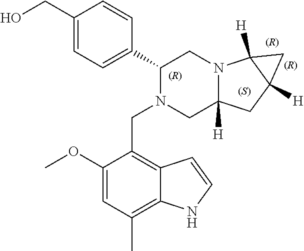 | 71900.89 | |
| 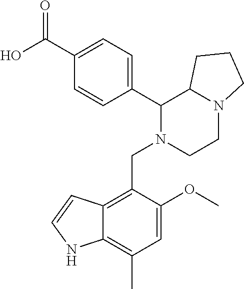 [SMILES: COc1cc(C)c2[nH]ccc2c1CN1CCN2CCCC2C1c1ccc(C(=O)O)cc1] | 834.904 | |
| 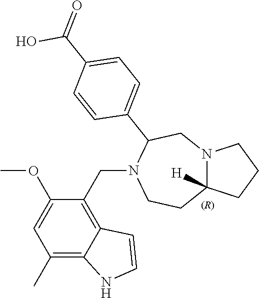 | 2367.479 | |
| 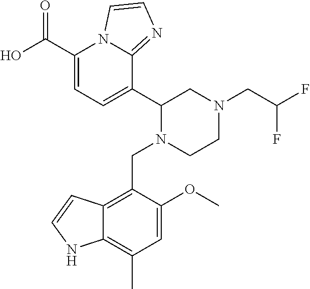 [SMILES: COc1cc(C)c2[nH]ccc2c1CN1CCN(CC(F)F)CC1c1ccc(C(=O)O)n2ccnc12] | 8141.798 | |
| 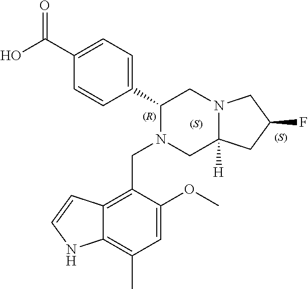 | 573.857 | |
| 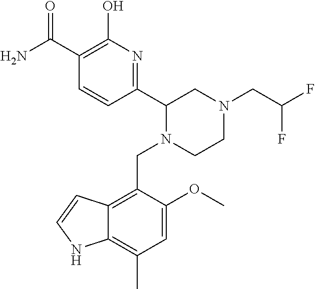 [SMILES: COc1cc(C)c2[nH]ccc2c1CN1CCN(CC(F)F)CC1c1ccc(C(N)=O)c(O)n1] | 10206.01 | |
| 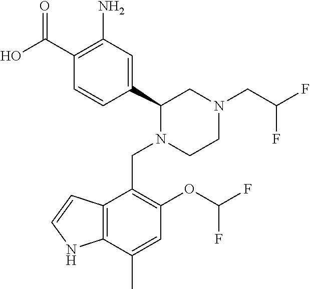 [SMILES: Cc1cc(OC(F)F)c(CN2CCN(CC(F)F)C[C@@H]2c2ccc(C(=O)O)c(N)c2)c2cc[nH]c12] | 106426 | |
| 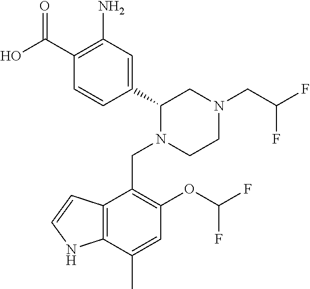 [SMILES: Cc1cc(OC(F)F)c(CN2CCN(CC(F)F)C[C@H]2c2ccc(C(=O)O)c(N)c2)c2cc[nH]c12] | 2690.204 | |
| 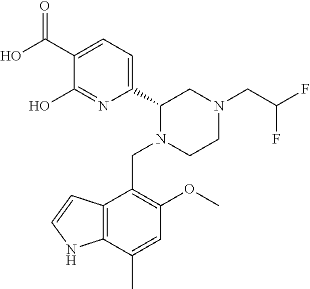 [SMILES: COc1cc(C)c2[nH]ccc2c1CN1CCN(CC(F)F)C[C@H]1c1ccc(C(=O)O)c(O)n1] | 1835.466 | |
| 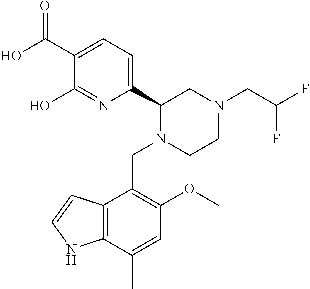 [SMILES: COc1cc(C)c2[nH]ccc2c1CN1CCN(CC(F)F)C[C@@H]1c1ccc(C(=O)O)c(O)n1] | 20071.11 | |
| 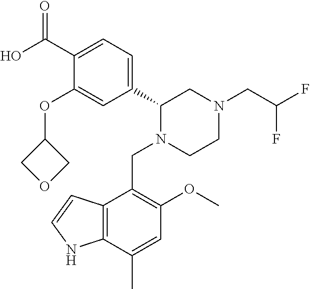 [SMILES: COc1cc(C)c2[nH]ccc2c1CN1CCN(CC(F)F)C[C@H]1c1ccc(C(=O)O)c(OC2COC2)c1] | 2177.935 | |
| 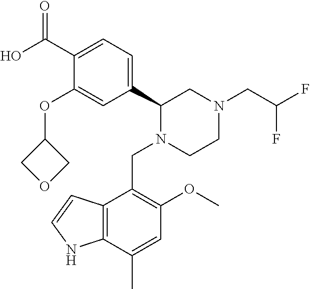 [SMILES: COc1cc(C)c2[nH]ccc2c1CN1CCN(CC(F)F)C[C@@H]1c1ccc(C(=O)O)c(OC2COC2)c1] | 7118.088 | |
| 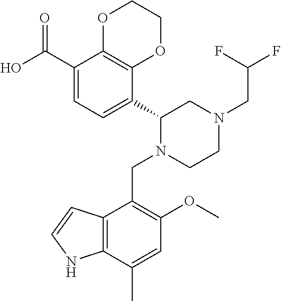 [SMILES: COc1cc(C)c2[nH]ccc2c1CN1CCN(CC(F)F)C[C@H]1c1ccc(C(=O)O)c2c1OCCO2] | 343.014 | |
| 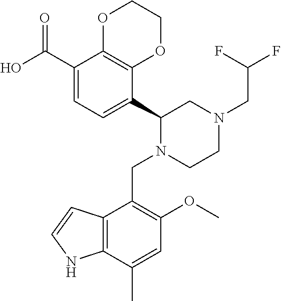 [SMILES: COc1cc(C)c2[nH]ccc2c1CN1CCN(CC(F)F)C[C@@H]1c1ccc(C(=O)O)c2c1OCCO2] | 96936.31 | |
| 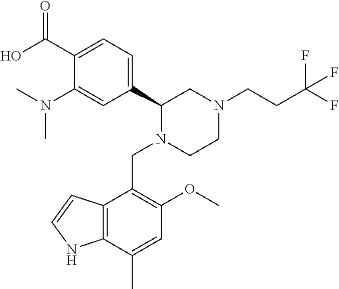 [SMILES: COc1cc(C)c2[nH]ccc2c1CN1CCN(CCC(F)(F)F)C[C@@H]1c1ccc(C(=O)O)c(N(C)C)c1] | 29071.14 | |
| 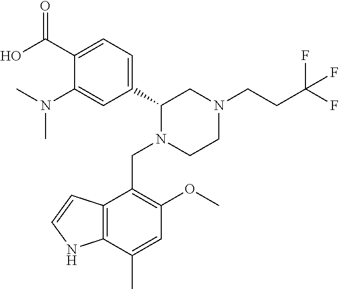 [SMILES: COc1cc(C)c2[nH]ccc2c1CN1CCN(CCC(F)(F)F)C[C@H]1c1ccc(C(=O)O)c(N(C)C)c1] | 636.749 | |
| 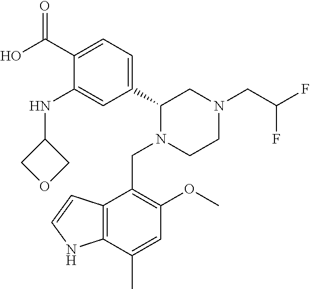 [SMILES: COc1cc(C)c2[nH]ccc2c1CN1CCN(CC(F)F)C[C@H]1c1ccc(C(=O)O)c(NC2COC2)c1] | 273.456 | |
| 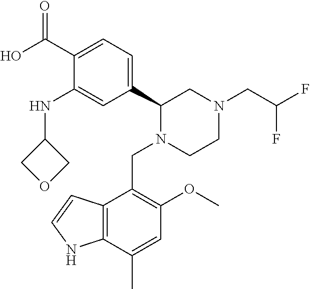 [SMILES: COc1cc(C)c2[nH]ccc2c1CN1CCN(CC(F)F)C[C@@H]1c1ccc(C(=O)O)c(NC2COC2)c1] | 5577.765 | |
| 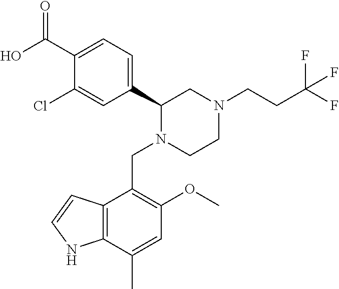 [SMILES: COc1cc(C)c2[nH]ccc2c1CN1CCN(CCC(F)(F)F)C[C@@H]1c1ccc(C(=O)O)c(Cl)c1] | 45365.48 | |
| 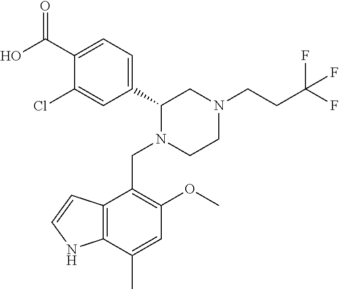 [SMILES: COc1cc(C)c2[nH]ccc2c1CN1CCN(CCC(F)(F)F)C[C@H]1c1ccc(C(=O)O)c(Cl)c1] | 645.019 | |
| 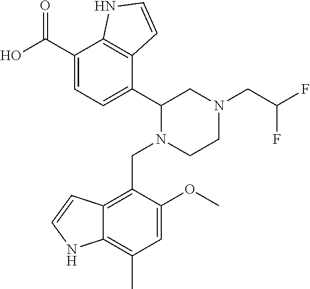 [SMILES: COc1cc(C)c2[nH]ccc2c1CN1CCN(CC(F)F)CC1c1ccc(C(=O)O)c2[nH]ccc12] | 339.746 | |
| 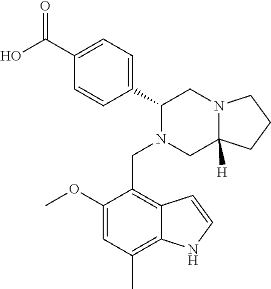 [SMILES: COc1cc(C)c2[nH]ccc2c1CN1C[C@H]2CCCN2C[C@H]1c1ccc(C(=O)O)cc1] | 3821.148 | |
| 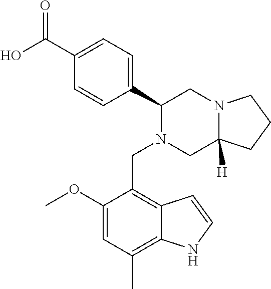 [SMILES: COc1cc(C)c2[nH]ccc2c1CN1C[C@H]2CCCN2C[C@@H]1c1ccc(C(=O)O)cc1] | 15640.27 | |
| 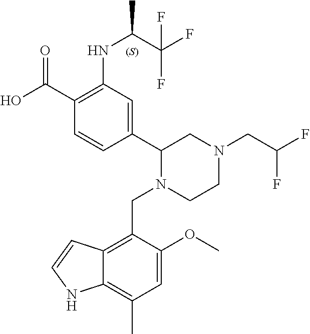 | 940.3 | |
| 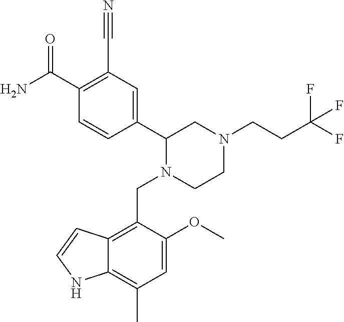 [SMILES: COc1cc(C)c2[nH]ccc2c1CN1CCN(CCC(F)(F)F)CC1c1ccc(C(N)=O)c(C#N)c1] | 23384.52 | |
| 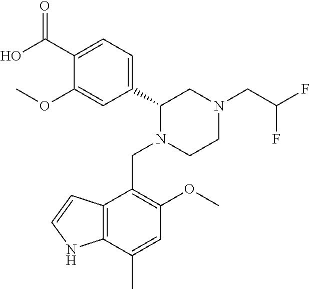 [SMILES: COc1cc([C@@H]2CN(CC(F)F)CCN2Cc2c(OC)cc(C)c3[nH]ccc23)ccc1C(=O)O] | 1126.33 | |
| 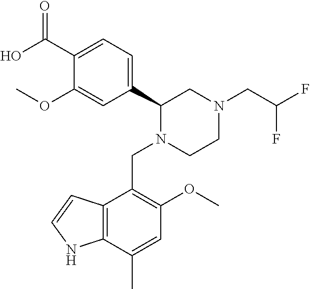 [SMILES: COc1cc([C@H]2CN(CC(F)F)CCN2Cc2c(OC)cc(C)c3[nH]ccc23)ccc1C(=O)O] | 7019.417 | |
| 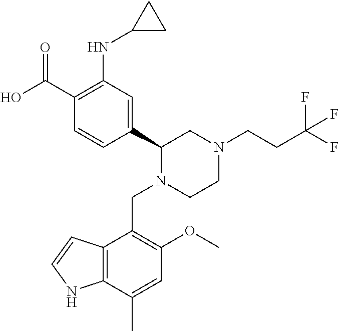 [SMILES: COc1cc(C)c2[nH]ccc2c1CN1CCN(CCC(F)(F)F)C[C@@H]1c1ccc(C(=O)O)c(NC2CC2)c1] | 17243.43 | |
| 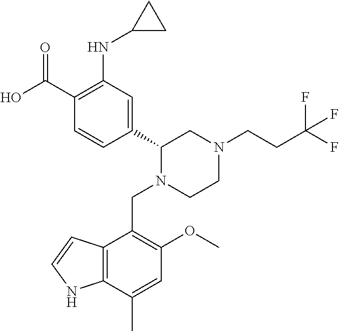 [SMILES: COc1cc(C)c2[nH]ccc2c1CN1CCN(CCC(F)(F)F)C[C@H]1c1ccc(C(=O)O)c(NC2CC2)c1] | 233.545 | |
| 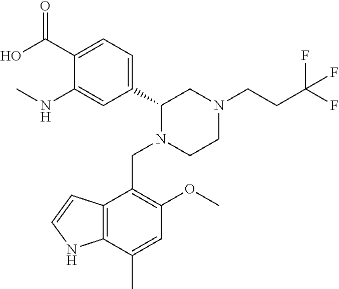 [SMILES: CNc1cc([C@@H]2CN(CCC(F)(F)F)CCN2Cc2c(OC)cc(C)c3[nH]ccc23)ccc1C(=O)O] | 39.829 | |
| 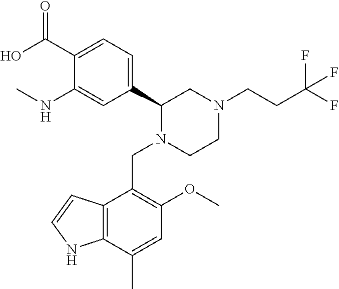 [SMILES: CNc1cc([C@H]2CN(CCC(F)(F)F)CCN2Cc2c(OC)cc(C)c3[nH]ccc23)ccc1C(=O)O] | 38617.18 | |
| 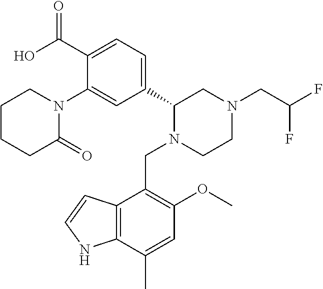 [SMILES: COc1cc(C)c2[nH]ccc2c1CN1CCN(CC(F)F)C[C@H]1c1ccc(C(=O)O)c(N2CCCCC2=O)c1] | 1052.247 | |
| 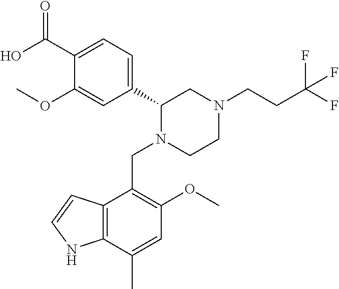 [SMILES: COc1cc([C@@H]2CN(CCC(F)(F)F)CCN2Cc2c(OC)cc(C)c3[nH]ccc23)ccc1C(=O)O] | 267.281 | |
| 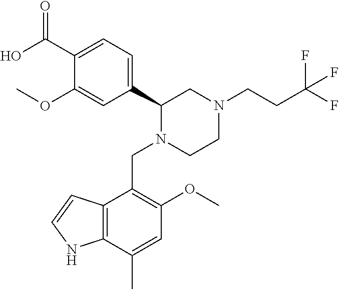 [SMILES: COc1cc([C@H]2CN(CCC(F)(F)F)CCN2Cc2c(OC)cc(C)c3[nH]ccc23)ccc1C(=O)O] | 27674.09 | |
| 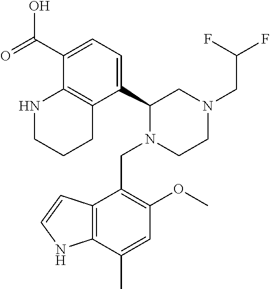 [SMILES: COc1cc(C)c2[nH]ccc2c1CN1CCN(CC(F)F)C[C@@H]1c1ccc(C(=O)O)c2c1CCCN2] | 15089.89 | |
| 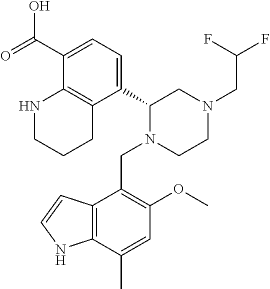 [SMILES: COc1cc(C)c2[nH]ccc2c1CN1CCN(CC(F)F)C[C@H]1c1ccc(C(=O)O)c2c1CCCN2] | 166.133 | |
| 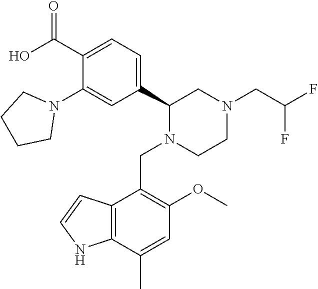 [SMILES: COc1cc(C)c2[nH]ccc2c1CN1CCN(CC(F)F)C[C@@H]1c1ccc(C(=O)O)c(N2CCCC2)c1] | | |
| 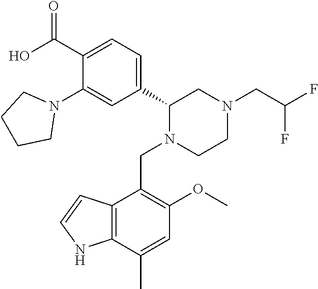 [SMILES: COc1cc(C)c2[nH]ccc2c1CN1CCN(CC(F)F)C[C@H]1c1ccc(C(=O)O)c(N2CCCC2)c1] | 810.097 | |
| 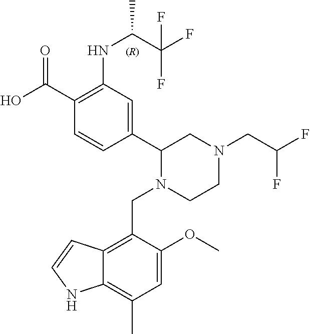 | 1159.173 | |
| 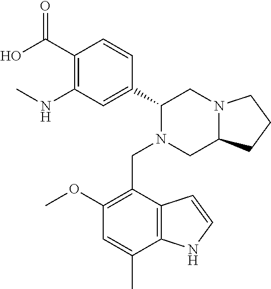 [SMILES: CNc1cc([C@@H]2CN3CCC[C@H]3CN2Cc2c(OC)cc(C)c3[nH]ccc23)ccc1C(=O)O] | 1525.329 | |
| 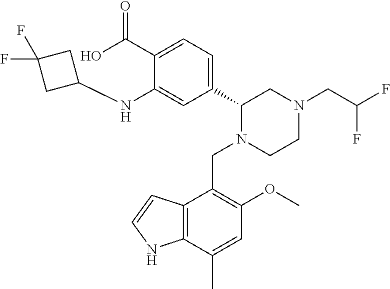 [SMILES: COc1cc(C)c2[nH]ccc2c1CN1CCN(CC(F)F)C[C@H]1c1ccc(C(=O)O)c(NC2CC(F)(F)C2)c1] | 808.126 | |
| 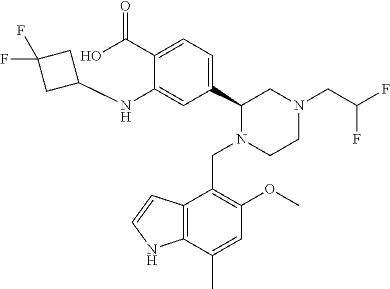 [SMILES: COc1cc(C)c2[nH]ccc2c1CN1CCN(CC(F)F)C[C@@H]1c1ccc(C(=O)O)c(NC2CC(F)(F)C2)c1] | 29865.92 | |
| 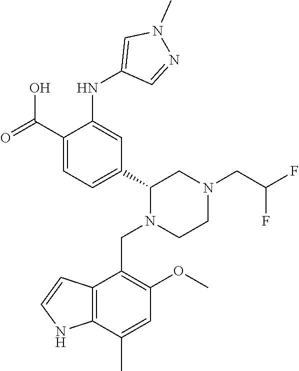 [SMILES: COc1cc(C)c2[nH]ccc2c1CN1CCN(CC(F)F)C[C@H]1c1ccc(C(=O)O)c(Nc2cnn(C)c2)c1] | 530.835 | |
| 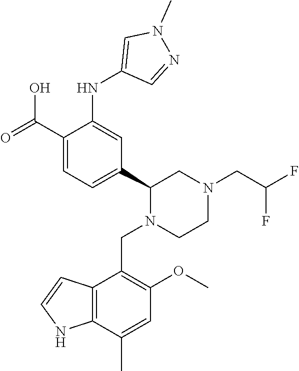 [SMILES: COc1cc(C)c2[nH]ccc2c1CN1CCN(CC(F)F)C[C@@H]1c1ccc(C(=O)O)c(Nc2cnn(C)c2)c1] | 9242.051 | |
| 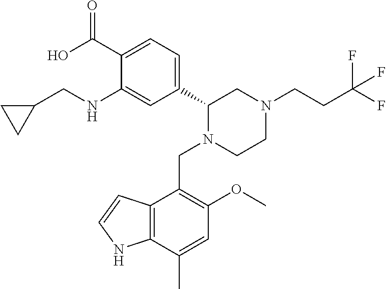 [SMILES: COc1cc(C)c2[nH]ccc2c1CN1CCN(CCC(F)(F)F)C[C@H]1c1ccc(C(=O)O)c(NCC2CC2)c1] | 461.418 | |
| 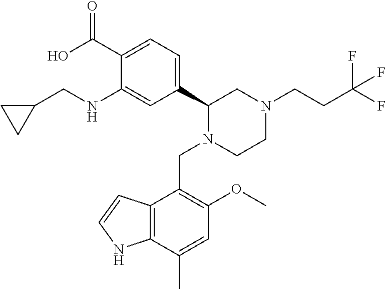 [SMILES: COc1cc(C)c2[nH]ccc2c1CN1CCN(CCC(F)(F)F)C[C@@H]1c1ccc(C(=O)O)c(NCC2CC2)c1] | 34941.7 | |
| 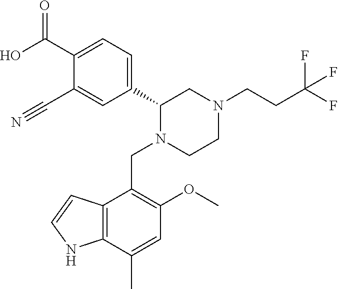 [SMILES: COc1cc(C)c2[nH]ccc2c1CN1CCN(CCC(F)(F)F)C[C@H]1c1ccc(C(=O)O)c(C#N)c1] | 766.05 | |
| 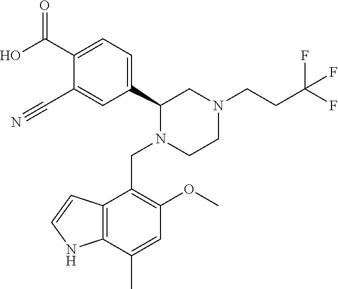 [SMILES: COc1cc(C)c2[nH]ccc2c1CN1CCN(CCC(F)(F)F)C[C@@H]1c1ccc(C(=O)O)c(C#N)c1] | 54899.84 | |
| 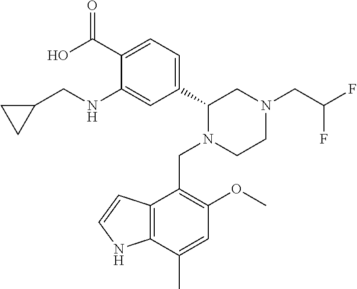 [SMILES: COc1cc(C)c2[nH]ccc2c1CN1CCN(CC(F)F)C[C@H]1c1ccc(C(=O)O)c(NCC2CC2)c1] | 766.68 | |
| 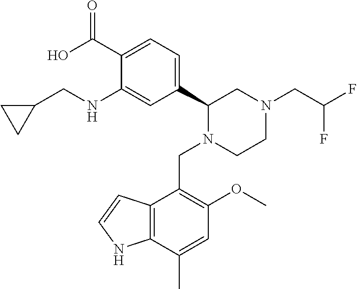 [SMILES: COc1cc(C)c2[nH]ccc2c1CN1CCN(CC(F)F)C[C@@H]1c1ccc(C(=O)O)c(NCC2CC2)c1] | 40851.95 | |
| 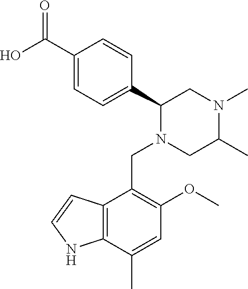 [SMILES: COc1cc(C)c2[nH]ccc2c1CN1CC(C)N(C)C[C@@H]1c1ccc(C(=O)O)cc1] | 18897.11 | |
| 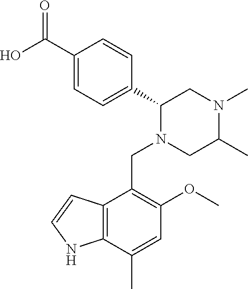 [SMILES: COc1cc(C)c2[nH]ccc2c1CN1CC(C)N(C)C[C@H]1c1ccc(C(=O)O)cc1] | 1762.879 | |
| 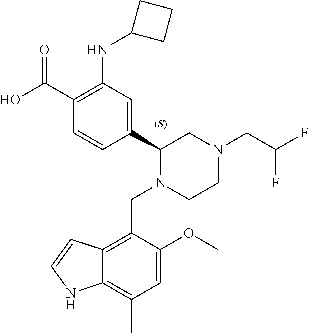 | 57058.83 | |
| 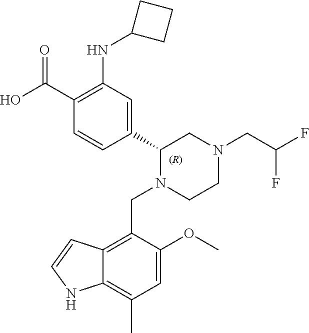 | 601.072 | |
| 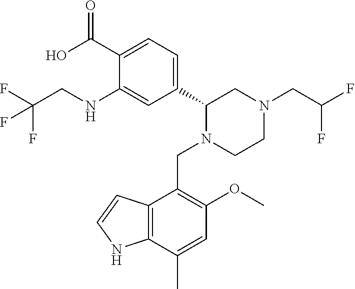 [SMILES: COc1cc(C)c2[nH]ccc2c1CN1CCN(CC(F)F)C[C@H]1c1ccc(C(=O)O)c(NCC(F)(F)F)c1] | 1090.785 | |
| 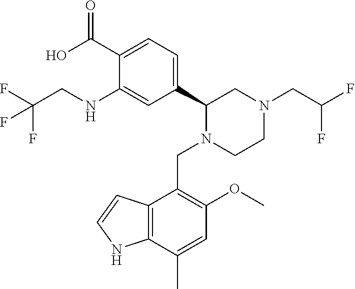 [SMILES: COc1cc(C)c2[nH]ccc2c1CN1CCN(CC(F)F)C[C@@H]1c1ccc(C(=O)O)c(NCC(F)(F)F)c1] | 77567.95 | |
| 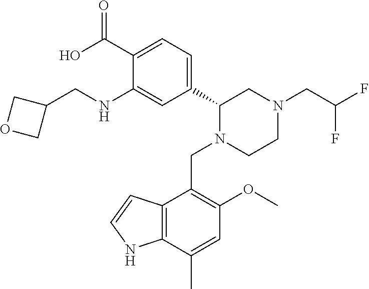 [SMILES: COc1cc(C)c2[nH]ccc2c1CN1CCN(CC(F)F)C[C@H]1c1ccc(C(=O)O)c(NCC2COC2)c1] | 400.36 | |
| 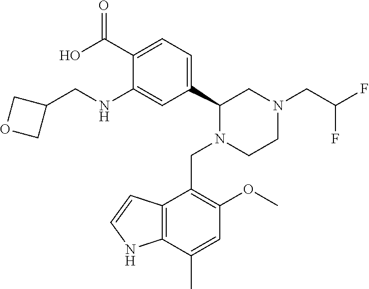 [SMILES: COc1cc(C)c2[nH]ccc2c1CN1CCN(CC(F)F)C[C@@H]1c1ccc(C(=O)O)c(NCC2COC2)c1] | 68212.31 | |
| 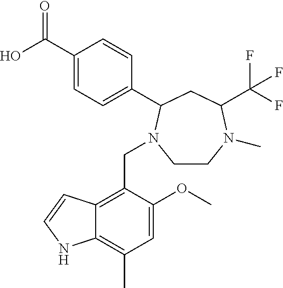 [SMILES: COc1cc(C)c2[nH]ccc2c1CN1CCN(C)C(C(F)(F)F)CC1c1ccc(C(=O)O)cc1] | 34333 | |
| 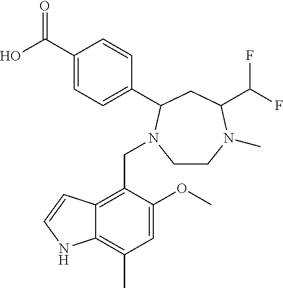 [SMILES: COc1cc(C)c2[nH]ccc2c1CN1CCN(C)C(C(F)F)CC1c1ccc(C(=O)O)cc1] | 9291.213 | |
| 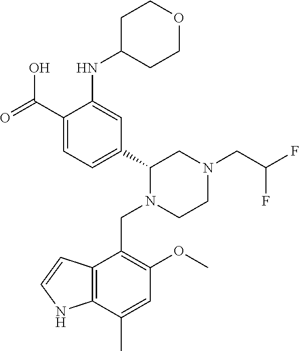 [SMILES: COc1cc(C)c2[nH]ccc2c1CN1CCN(CC(F)F)C[C@H]1c1ccc(C(=O)O)c(NC2CCOCC2)c1] | 156.625 | |
| 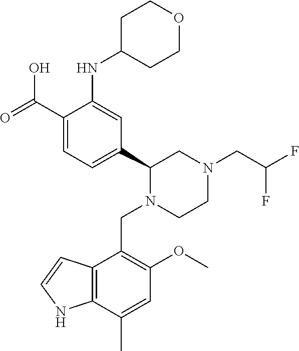 [SMILES: COc1cc(C)c2[nH]ccc2c1CN1CCN(CC(F)F)C[C@@H]1c1ccc(C(=O)O)c(NC2CCOCC2)c1] | 46203.03 | |
| 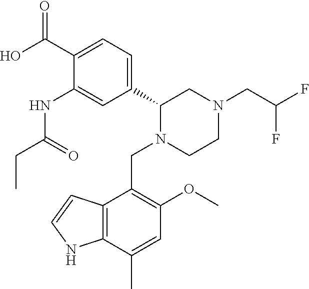 [SMILES: CCC(=O)Nc1cc([C@@H]2CN(CC(F)F)CCN2Cc2c(OC)cc(C)c3[nH]ccc23)ccc1C(=O)O] | 1259.334 | |
| 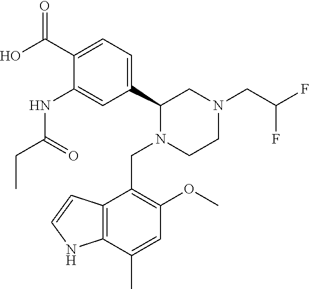 [SMILES: CCC(=O)Nc1cc([C@H]2CN(CC(F)F)CCN2Cc2c(OC)cc(C)c3[nH]ccc23)ccc1C(=O)O] | 118332.4 | |
| 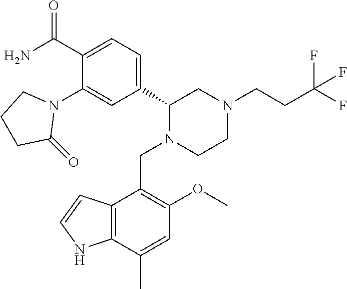 [SMILES: COc1cc(C)c2[nH]ccc2c1CN1CCN(CCC(F)(F)F)C[C@H]1c1ccc(C(N)=O)c(N2CCCC2=O)c1] | 17598.12 | |
| 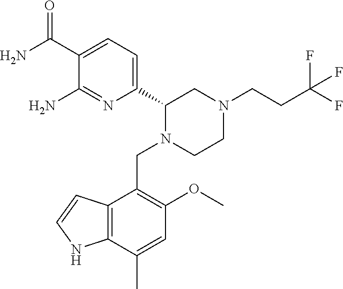 [SMILES: COc1cc(C)c2[nH]ccc2c1CN1CCN(CCC(F)(F)F)C[C@H]1c1ccc(C(N)=O)c(N)n1] | 37530.86 | |
| 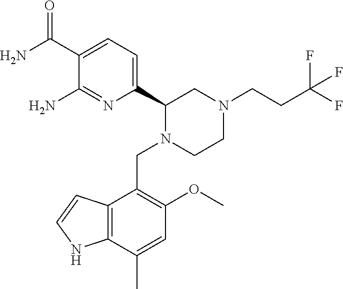 [SMILES: COc1cc(C)c2[nH]ccc2c1CN1CCN(CCC(F)(F)F)C[C@@H]1c1ccc(C(N)=O)c(N)n1] | 102446.9 | |
| 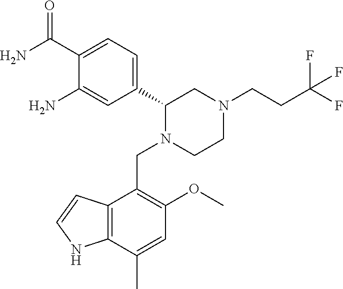 [SMILES: COc1cc(C)c2[nH]ccc2c1CN1CCN(CCC(F)(F)F)C[C@H]1c1ccc(C(N)=O)c(N)c1] | 3857.921 | |
| 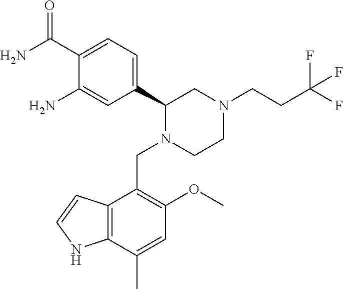 [SMILES: COc1cc(C)c2[nH]ccc2c1CN1CCN(CCC(F)(F)F)C[C@@H]1c1ccc(C(N)=O)c(N)c1] | 119290.4 | |
| 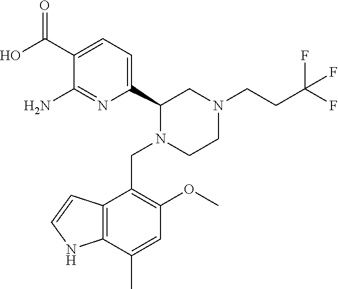 [SMILES: COc1cc(C)c2[nH]ccc2c1CN1CCN(CCC(F)(F)F)C[C@@H]1c1ccc(C(=O)O)c(N)n1] | 15193.85 | |
| 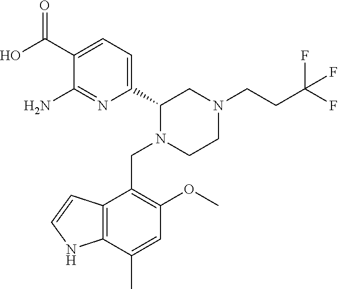 [SMILES: COc1cc(C)c2[nH]ccc2c1CN1CCN(CCC(F)(F)F)C[C@H]1c1ccc(C(=O)O)c(N)n1] | 248.58 | |
| 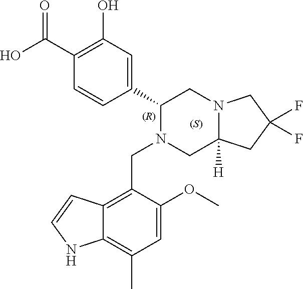 | 25182 | |
| 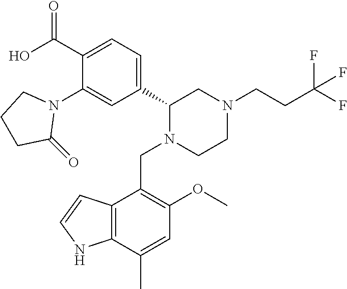 [SMILES: COc1cc(C)c2[nH]ccc2c1CN1CCN(CCC(F)(F)F)C[C@H]1c1ccc(C(=O)O)c(N2CCCC2=O)c1] | 1241.056 | |
| 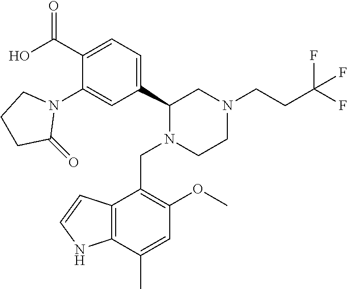 [SMILES: COc1cc(C)c2[nH]ccc2c1CN1CCN(CCC(F)(F)F)C[C@@H]1c1ccc(C(=O)O)c(N2CCCC2=O)c1] | 7034.277 | |
| 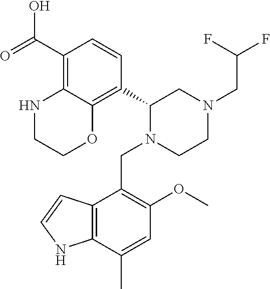 [SMILES: COc1cc(C)c2[nH]ccc2c1CN1CCN(CC(F)F)C[C@H]1c1ccc(C(=O)O)c2c1OCCN2] | 148.096 | 0.038 |
| 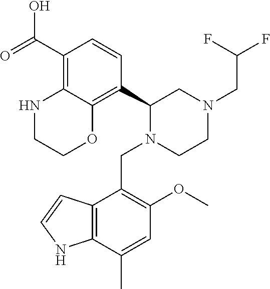 [SMILES: COc1cc(C)c2[nH]ccc2c1CN1CCN(CC(F)F)C[C@@H]1c1ccc(C(=O)O)c2c1OCCN2] | 20801.57 | 10 |
| 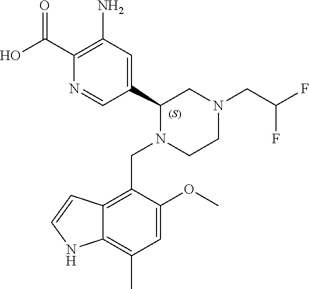 | 51450.27 | |
| 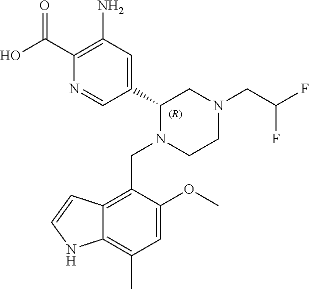 | 2279.603 | |
| 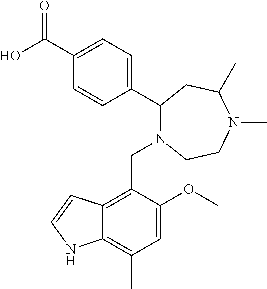 [SMILES: COc1cc(C)c2[nH]ccc2c1CN1CCN(C)C(C)CC1c1ccc(C(=O)O)cc1] | 8529.132 | |
| 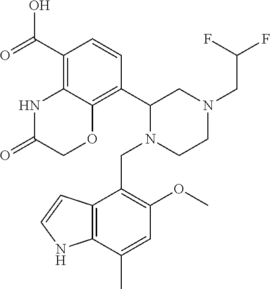 [SMILES: COc1cc(C)c2[nH]ccc2c1CN1CCN(CC(F)F)CC1c1ccc(C(=O)O)c2c1OCC(=O)N2] | 988.706 | 0.128 |
| 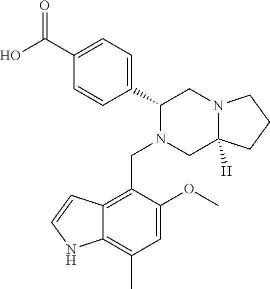 [SMILES: COc1cc(C)c2[nH]ccc2c1CN1C[C@@H]2CCCN2C[C@H]1c1ccc(C(=O)O)cc1] | 1180.921 | 0.129 |
| 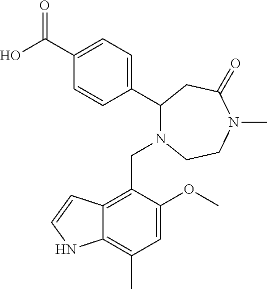 [SMILES: COc1cc(C)c2[nH]ccc2c1CN1CCN(C)C(=O)CC1c1ccc(C(=O)O)cc1] | 34192.83 | |
| 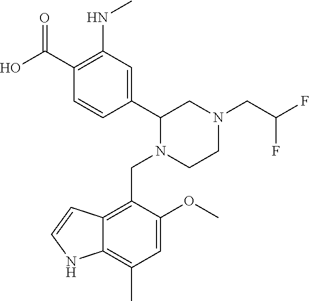 [SMILES: CNc1cc(C2CN(CC(F)F)CCN2Cc2c(OC)cc(C)c3[nH]ccc23)ccc1C(=O)O] | 262.619 | 0.062 |
| 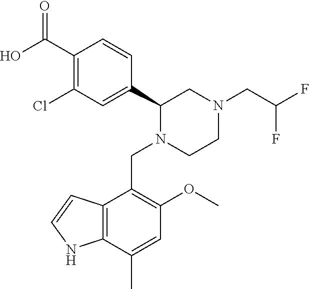 [SMILES: COc1cc(C)c2[nH]ccc2c1CN1CCN(CC(F)F)C[C@@H]1c1ccc(C(=O)O)c(Cl)c1] | 4079.999 | |
| 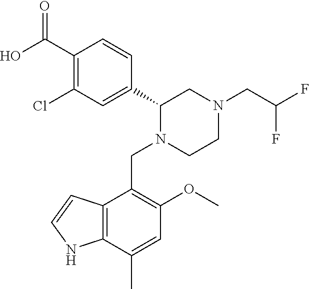 [SMILES: COc1cc(C)c2[nH]ccc2c1CN1CCN(CC(F)F)C[C@H]1c1ccc(C(=O)O)c(Cl)c1] | 496.519 | 0.092 |
| 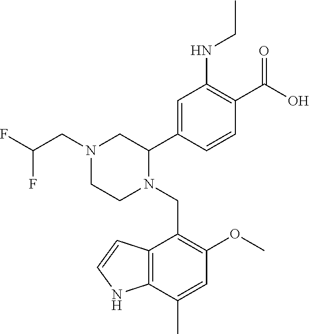 [SMILES: CCNc1cc(C2CN(CC(F)F)CCN2Cc2c(OC)cc(C)c3[nH]ccc23)ccc1C(=O)O] | 82.741 | 0.054 |
| 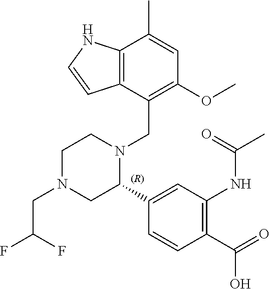 | 153.479 | 0.021 |
| 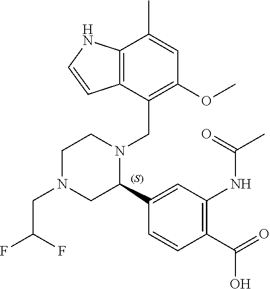 | 13211.31 | |
| 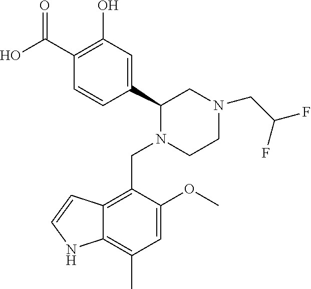 [SMILES: COc1cc(C)c2[nH]ccc2c1CN1CCN(CC(F)F)C[C@@H]1c1ccc(C(=O)O)c(O)c1] | 4196.757 | 8.078 |
| 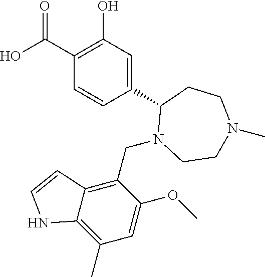 [SMILES: COc1cc(C)c2[nH]ccc2c1CN1CCN(C)CC[C@H]1c1ccc(C(=O)O)c(O)c1] | 37.148 | 0.025 |
| 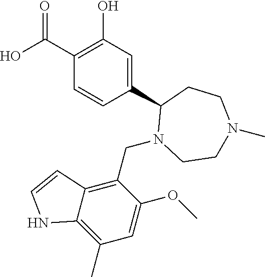 [SMILES: COc1cc(C)c2[nH]ccc2c1CN1CCN(C)CC[C@@H]1c1ccc(C(=O)O)c(O)c1] | 1378.335 | |
| 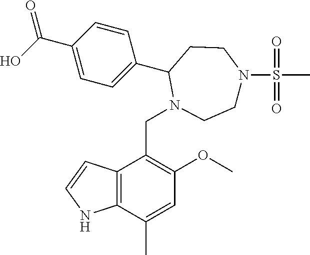 [SMILES: COc1cc(C)c2[nH]ccc2c1CN1CCN(S(C)(=O)=O)CCC1c1ccc(C(=O)O)cc1] | 21290.51 | 9.898 |
| 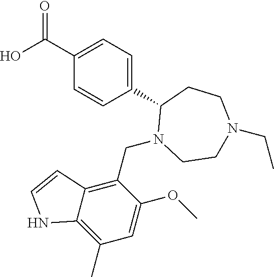 [SMILES: CCN1CC[C@@H](c2ccc(C(=O)O)cc2)N(Cc2c(OC)cc(C)c3[nH]ccc23)CC1] | 148.869 | 0.051 |
| 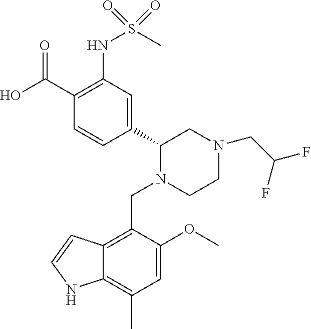 [SMILES: COc1cc(C)c2[nH]ccc2c1CN1CCN(CC(F)F)C[C@H]1c1ccc(C(=O)O)c(NS(C)(=O)=O)c1] | 81.748 | 0.095 |
| 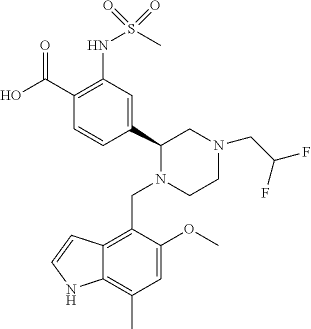 [SMILES: COc1cc(C)c2[nH]ccc2c1CN1CCN(CC(F)F)C[C@@H]1c1ccc(C(=O)O)c(NS(C)(=O)=O)c1] | 16283.96 | 20 |
| 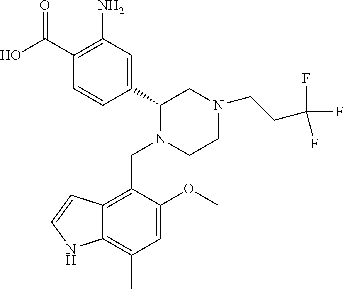 [SMILES: COc1cc(C)c2[nH]ccc2c1CN1CCN(CCC(F)(F)F)C[C@H]1c1ccc(C(=O)O)c(N)c1] | 12.502 | 0.022 |
| 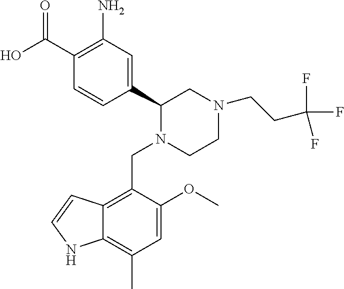 [SMILES: COc1cc(C)c2[nH]ccc2c1CN1CCN(CCC(F)(F)F)C[C@@H]1c1ccc(C(=O)O)c(N)c1] | 10240.15 | 1.732 |
| 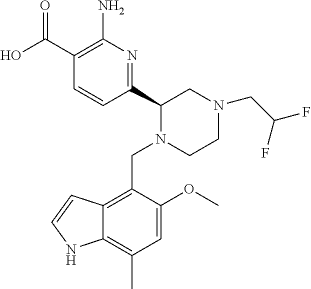 [SMILES: COc1cc(C)c2[nH]ccc2c1CN1CCN(CC(F)F)C[C@@H]1c1ccc(C(=O)O)c(N)n1] | 20423.5 | 6.282 |
| 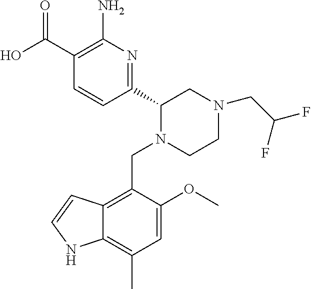 [SMILES: COc1cc(C)c2[nH]ccc2c1CN1CCN(CC(F)F)C[C@H]1c1ccc(C(=O)O)c(N)n1] | 453.204 | 0.147 |
| 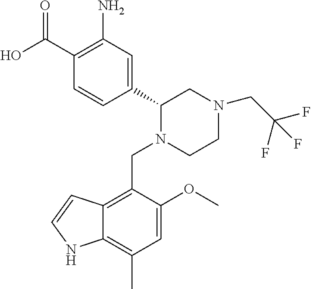 [SMILES: COc1cc(C)c2[nH]ccc2c1CN1CCN(CC(F)(F)F)C[C@H]1c1ccc(C(=O)O)c(N)c1] | 36.361 | 0.049 |
| 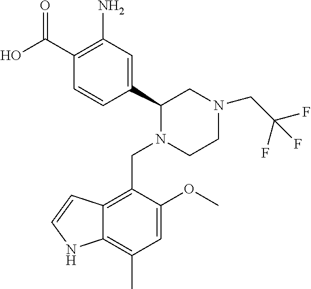 [SMILES: COc1cc(C)c2[nH]ccc2c1CN1CCN(CC(F)(F)F)C[C@@H]1c1ccc(C(=O)O)c(N)c1] | 9627.189 | 20 |
| 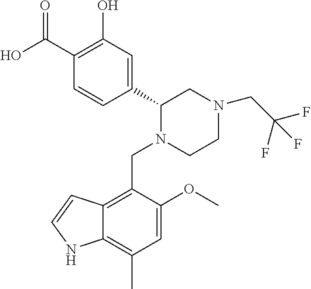 [SMILES: COc1cc(C)c2[nH]ccc2c1CN1CCN(CC(F)(F)F)C[C@H]1c1ccc(C(=O)O)c(O)c1] | 282.151 | |
| 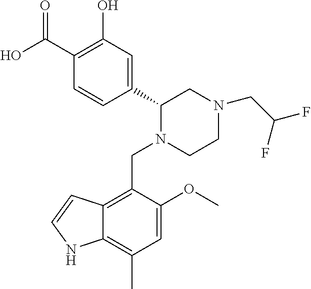 [SMILES: COc1cc(C)c2[nH]ccc2c1CN1CCN(CC(F)F)C[C@H]1c1ccc(C(=O)O)c(O)c1] | 202.284 | |
| 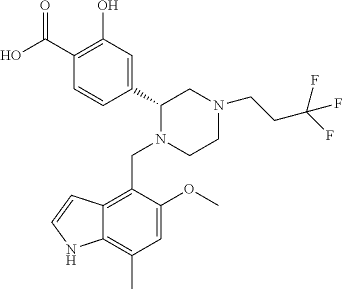 [SMILES: COc1cc(C)c2[nH]ccc2c1CN1CCN(CCC(F)(F)F)C[C@H]1c1ccc(C(=O)O)c(O)c1] | 47.77 | 0.083 |
| 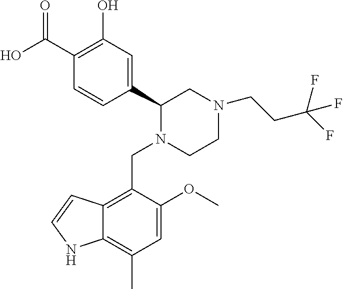 [SMILES: COc1cc(C)c2[nH]ccc2c1CN1CCN(CCC(F)(F)F)C[C@@H]1c1ccc(C(=O)O)c(O)c1] | 6319.379 | 3.545 |
| 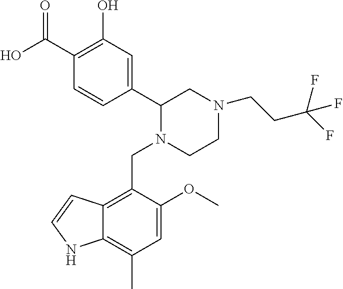 [SMILES: COc1cc(C)c2[nH]ccc2c1CN1CCN(CCC(F)(F)F)CC1c1ccc(C(=O)O)c(O)c1] | 116.974 | |
| 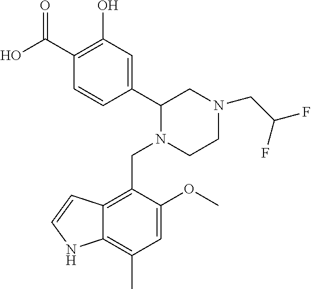 [SMILES: COc1cc(C)c2[nH]ccc2c1CN1CCN(CC(F)F)CC1c1ccc(C(=O)O)c(O)c1] | 264.57 | |
| 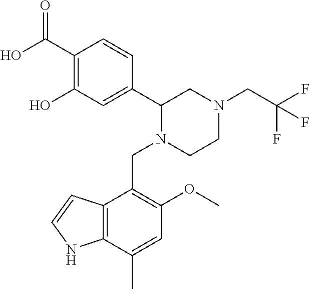 [SMILES: COc1cc(C)c2[nH]ccc2c1CN1CCN(CC(F)(F)F)CC1c1ccc(C(=O)O)c(O)c1] | 384.666 | |
| 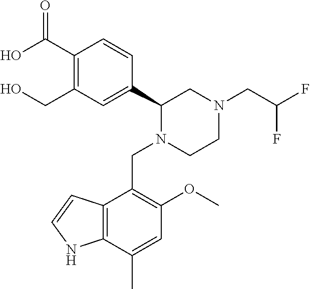 [SMILES: COc1cc(C)c2[nH]ccc2c1CN1CCN(CC(F)F)C[C@@H]1c1ccc(C(=O)O)c(CO)c1] | 4193.017 | |
| 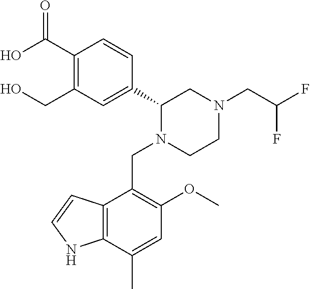 [SMILES: COc1cc(C)c2[nH]ccc2c1CN1CCN(CC(F)F)C[C@H]1c1ccc(C(=O)O)c(CO)c1] | 262.067 | |
| 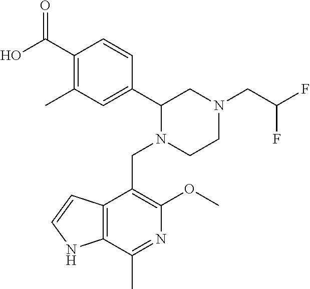 [SMILES: COc1nc(C)c2[nH]ccc2c1CN1CCN(CC(F)F)CC1c1ccc(C(=O)O)c(C)c1] | 22878.47 | 10 |
| 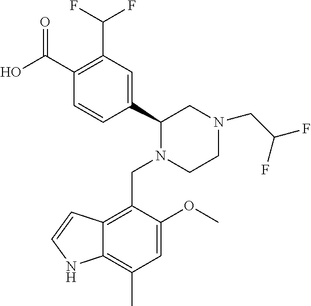 [SMILES: COc1cc(C)c2[nH]ccc2c1CN1CCN(CC(F)F)C[C@@H]1c1ccc(C(=O)O)c(C(F)F)c1] | 1355.661 | 10 |
| 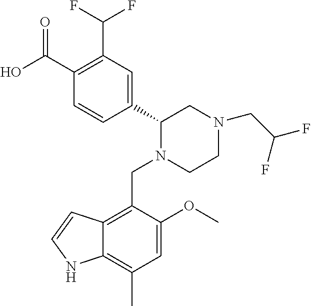 [SMILES: COc1cc(C)c2[nH]ccc2c1CN1CCN(CC(F)F)C[C@H]1c1ccc(C(=O)O)c(C(F)F)c1] | 324.145 | 3.139 |
| 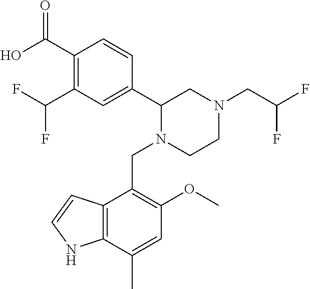 [SMILES: COc1cc(C)c2[nH]ccc2c1CN1CCN(CC(F)F)CC1c1ccc(C(=O)O)c(C(F)F)c1] | 856.808 | |
| 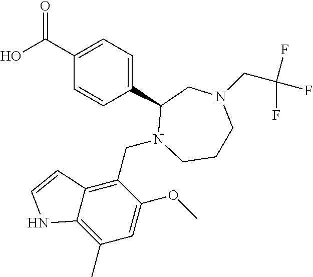 [SMILES: COc1cc(C)c2[nH]ccc2c1CN1CCCN(CC(F)(F)F)C[C@@H]1c1ccc(C(=O)O)cc1] | 973.225 | |
| 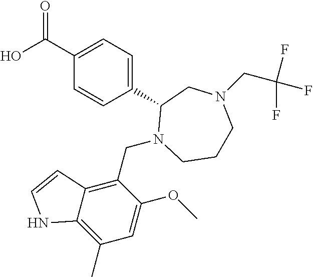 [SMILES: COc1cc(C)c2[nH]ccc2c1CN1CCCN(CC(F)(F)F)C[C@H]1c1ccc(C(=O)O)cc1] | 33.467 | |
| 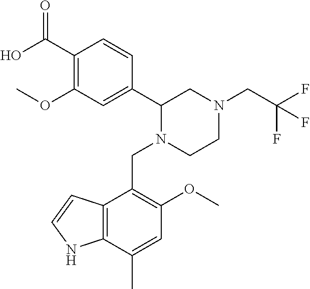 [SMILES: COc1cc(C2CN(CC(F)(F)F)CCN2Cc2c(OC)cc(C)c3[nH]ccc23)ccc1C(=O)O] | 691.199 | |
| 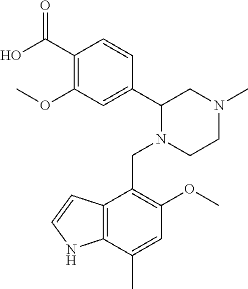 [SMILES: COc1cc(C2CN(C)CCN2Cc2c(OC)cc(C)c3[nH]ccc23)ccc1C(=O)O] | 415.176 | |
| 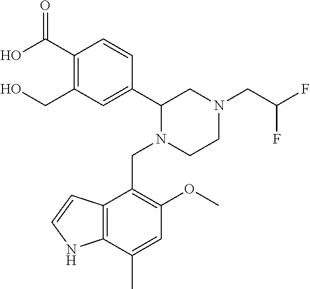 [SMILES: COc1cc(C)c2[nH]ccc2c1CN1CCN(CC(F)F)CC1c1ccc(C(=O)O)c(CO)c1] | 282.578 | 0.498 |
| 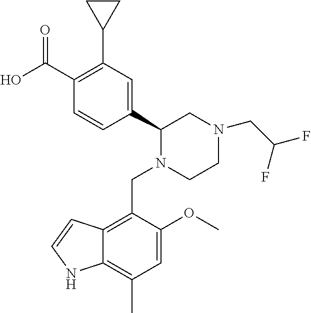 [SMILES: COc1cc(C)c2[nH]ccc2c1CN1CCN(CC(F)F)C[C@@H]1c1ccc(C(=O)O)c(C2CC2)c1] | 14600.34 | |
| 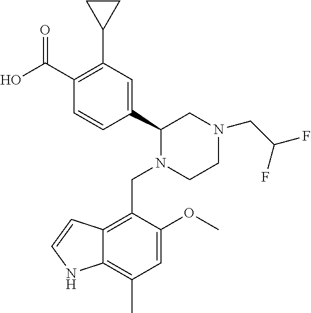 [SMILES: COc1cc(C)c2[nH]ccc2c1CN1CCN(CC(F)F)C[C@@H]1c1ccc(C(=O)O)c(C2CC2)c1] | 270.01 | 4.821 |
| 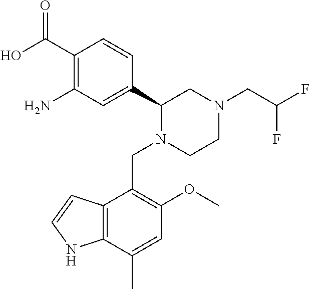 [SMILES: COc1cc(C)c2[nH]ccc2c1CN1CCN(CC(F)F)C[C@@H]1c1ccc(C(=O)O)c(N)c1] | 505.256 | |
| 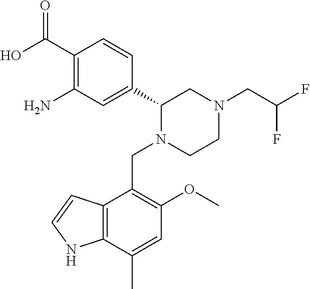 [SMILES: COc1cc(C)c2[nH]ccc2c1CN1CCN(CC(F)F)C[C@H]1c1ccc(C(=O)O)c(N)c1] | 58.236 | 0.059 |
| 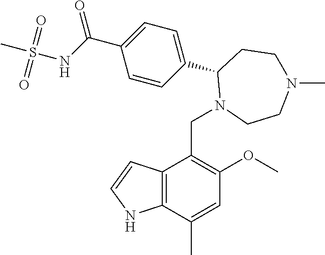 | 76.012 | 0.085 |
| 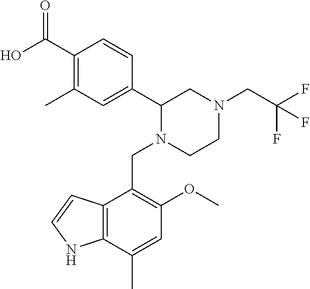 [SMILES: COc1cc(C)c2[nH]ccc2c1CN1CCN(CC(F)(F)F)CC1c1ccc(C(=O)O)c(C)c1] | 888.262 | |
| 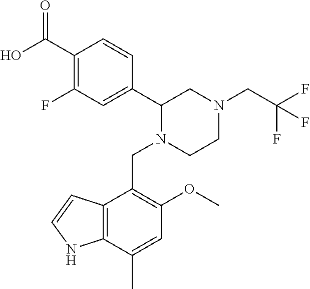 [SMILES: COc1cc(C)c2[nH]ccc2c1CN1CCN(CC(F)(F)F)CC1c1ccc(C(=O)O)c(F)c1] | 680.769 | |
| 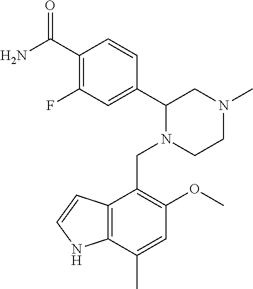 [SMILES: COc1cc(C)c2[nH]ccc2c1CN1CCN(C)CC1c1ccc(C(N)=O)c(F)c1] | 2325.422 | |
| 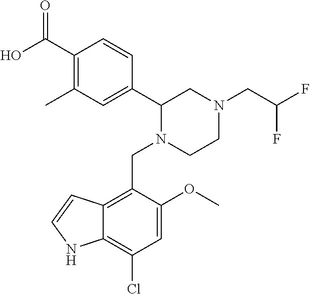 [SMILES: COc1cc(Cl)c2[nH]ccc2c1CN1CCN(CC(F)F)CC1c1ccc(C(=O)O)c(C)c1] | 9341.591 | |
| 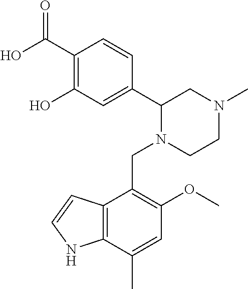 [SMILES: COc1cc(C)c2[nH]ccc2c1CN1CCN(C)CC1c1ccc(C(=O)O)c(O)c1] | 276.45 | |
| 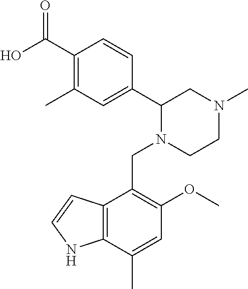 [SMILES: COc1cc(C)c2[nH]ccc2c1CN1CCN(C)CC1c1ccc(C(=O)O)c(C)c1] | 1156.63 | |
| 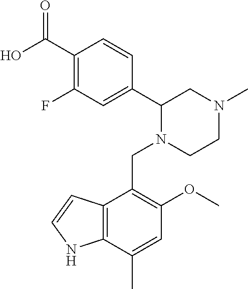 [SMILES: COc1cc(C)c2[nH]ccc2c1CN1CCN(C)CC1c1ccc(C(=O)O)c(F)c1] | 382.752 | |
| 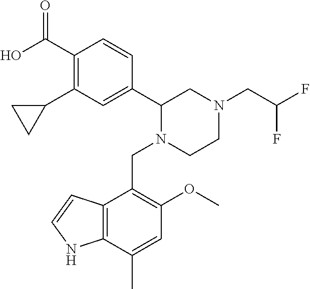 [SMILES: COc1cc(C)c2[nH]ccc2c1CN1CCN(CC(F)F)CC1c1ccc(C(=O)O)c(C2CC2)c1] | 1144.735 | |
| 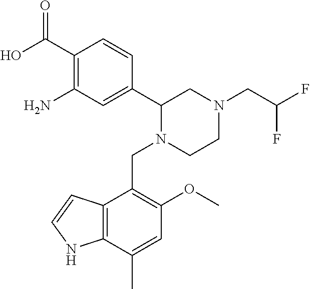 [SMILES: COc1cc(C)c2[nH]ccc2c1CN1CCN(CC(F)F)CC1c1ccc(C(=O)O)c(N)c1] | 102.508 | |
| 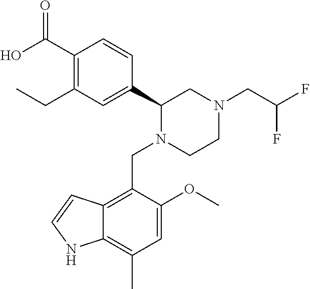 [SMILES: CCc1cc([C@H]2CN(CC(F)F)CCN2Cc2c(OC)cc(C)c3[nH]ccc23)ccc1C(=O)O] | 387.826 | 0.383 |
| 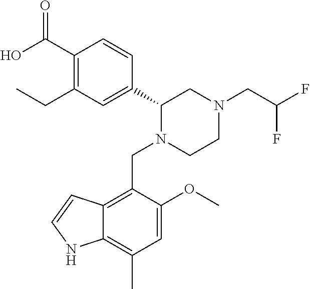 [SMILES: CCc1cc([C@@H]2CN(CC(F)F)CCN2Cc2c(OC)cc(C)c3[nH]ccc23)ccc1C(=O)O] | 47920.62 | |
| 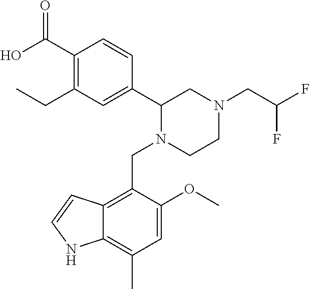 [SMILES: CCc1cc(C2CN(CC(F)F)CCN2Cc2c(OC)cc(C)c3[nH]ccc23)ccc1C(=O)O] | 579.965 | |
| 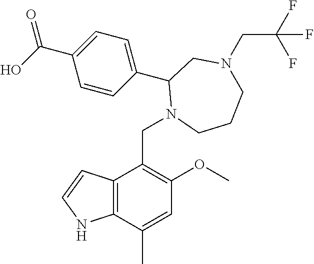 [SMILES: COc1cc(C)c2[nH]ccc2c1CN1CCCN(CC(F)(F)F)CC1c1ccc(C(=O)O)cc1] | 53.167 | 0.024 |
| 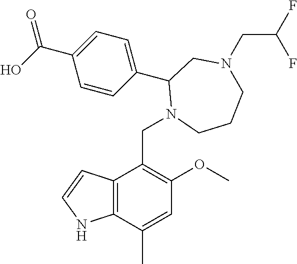 [SMILES: COc1cc(C)c2[nH]ccc2c1CN1CCCN(CC(F)F)CC1c1ccc(C(=O)O)cc1] | 83.118 | 0.05 |
| 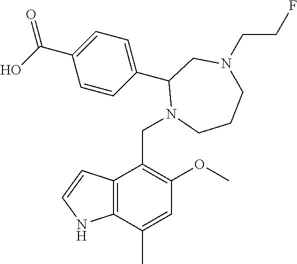 [SMILES: COc1cc(C)c2[nH]ccc2c1CN1CCCN(CCF)CC1c1ccc(C(=O)O)cc1] | 291.265 | |
| 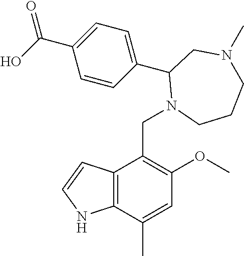 [SMILES: COc1cc(C)c2[nH]ccc2c1CN1CCCN(C)CC1c1ccc(C(=O)O)cc1] | 2258.734 | |
| 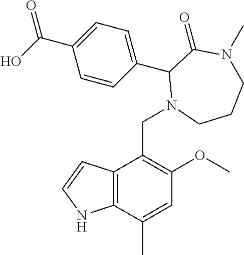 [SMILES: COc1cc(C)c2[nH]ccc2c1CN1CCCN(C)C(=O)C1c1ccc(C(=O)O)cc1] | 6146.334 | |
| 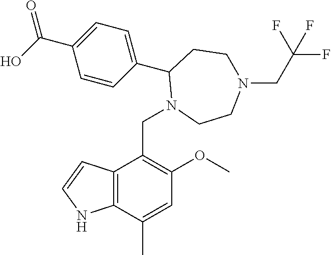 [SMILES: COc1cc(C)c2[nH]ccc2c1CN1CCN(CC(F)(F)F)CCC1c1ccc(C(=O)O)cc1] | 9471.598 | |
| 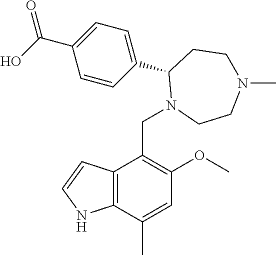 [SMILES: COc1cc(C)c2[nH]ccc2c1CN1CCN(C)CC[C@H]1c1ccc(C(=O)O)cc1] | 70.09 | 0.049 |
| 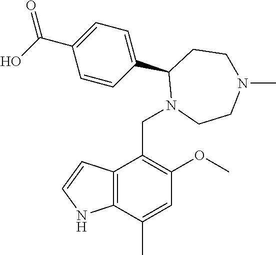 [SMILES: COc1cc(C)c2[nH]ccc2c1CN1CCN(C)CC[C@@H]1c1ccc(C(=O)O)cc1] | 3009.687 | 0.022 |

### EQUIVALENTS

Those skilled in the art will recognize, or be able to ascertain using no more than routine experimentation, many equivalents to specific embodiments of the present disclosure.

1. A compound, wherein the compound has the structure of formula I: 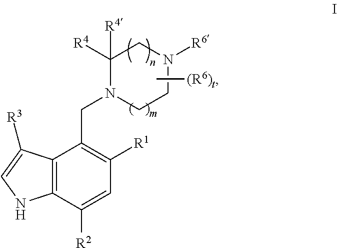
or a pharmaceutically acceptable salt thereof, wherein:
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R⁵ and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{4′} is hydrogen, C₁-C₄ alkyl, or hydroxy C₁-C₄ alkyl;
R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

2. The compound of claim 1, wherein R¹ is selected from C₁-C₆ alkoxy, C₁-C₆ alkoxy C₁-C₆ alkyl, C₁-C₆ alkoxy C₁-C₆ alkoxy, C₃-C₆ cycloalkyl C₁-C₆ alkoxy, and halo C₁-C₆ alkoxy.

3. The compound of claim 2, wherein R¹ is methoxy.

4. The compound of claim 2, wherein R² is selected from C₁-C₆ alkyl and halogen.

5. The compound claim 4, wherein R² is methyl.

6. The compound claim 4, wherein R³ is selected from hydrogen, halogen, and cyano.

7. The compound of claim 6, wherein R³ is hydrogen.

8. The compound of claim 6, wherein R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is substituted with R⁵ and further substituted with one or two R⁵′.

9. The compound of claim 6, wherein R⁴ is phenyl substituted with R⁵ and further optionally substituted with one or two R⁵′.

10. The compound of claim 6, wherein R⁴ is phenyl para-substituted (unless otherwise indicated, relative to the atom at which R⁴ is attached to the rest of the compound) with R⁵ and further optionally substituted with one or two R⁵′.

11. The compound of claim 10, wherein R⁵ is —C(O)OH.

12. The compound of claim 11, wherein R⁴ is substituted with one or two R⁵′.

13. The compound of claim 12, wherein a R⁵′ group is ortho relative to R⁵.

14. The compound of claim 13, wherein R⁵′ is —N(R⁵¹)₂.

15. The compound of claim 14, wherein R⁵′ is —NH₂.

16. The compound of any one of the preceding claims, wherein R⁴ is hydrogen.

17. The compound of claim 11, wherein two R⁶ on a carbon atom are taken together to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or wherein two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.

18. The compound of claim 11, wherein two R⁶ are taken together with their intervening atoms to form a 5-7 membered cycloalkyl ring, wherein the ring is optionally substituted with one or more substituents independently selected from halogen, C₁-C₄ alkyl, halo C₁-C₄ alkyl, C₁-C₄ alkoxy, hydroxyl, and oxo.

19. The compound of claim 11, wherein n is 1 and m is 2.

20. The compound of claim 11, wherein R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo.

21. The compound of claim 20, wherein R⁶ and R^{6′} are taken together with their intervening atoms to form

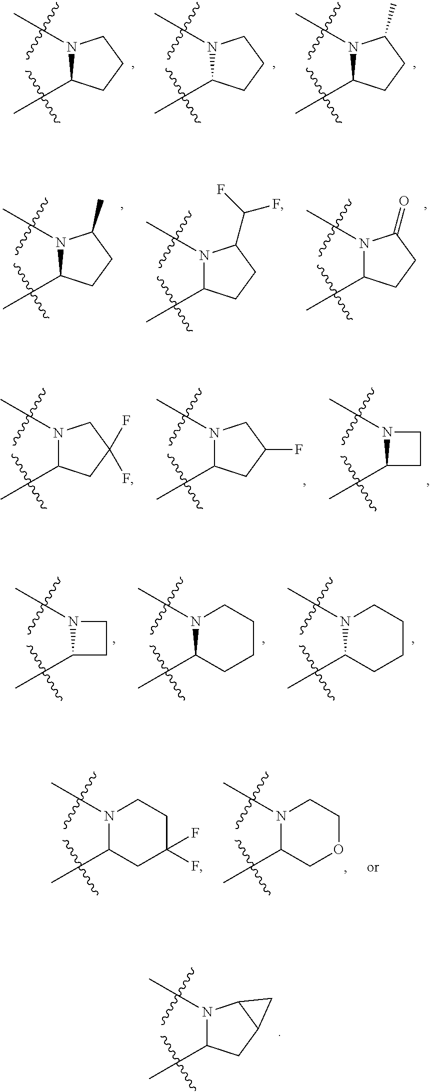

22. A compound, wherein the compound has the structure of formula M-I: 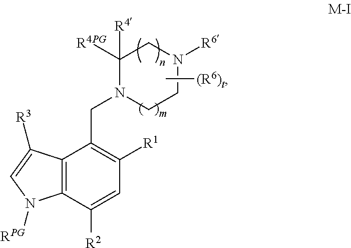
or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-II: 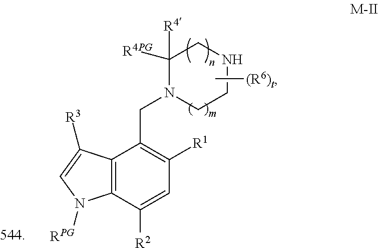
or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG}), or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-III: 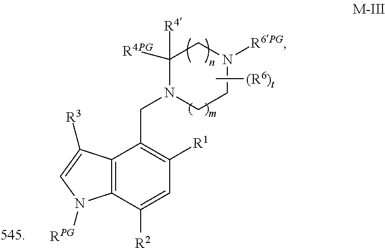
or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XII′: 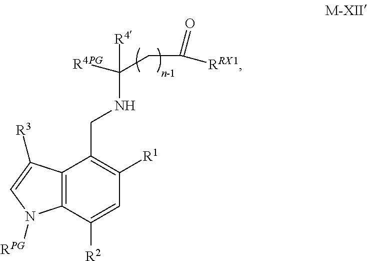
or a salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR⁶, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
R^{RX1} is —OH or —C(O)R^{RX1} is an activated carboxyl group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-X′: 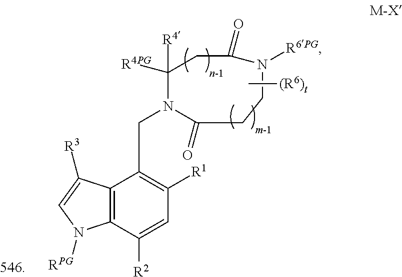
or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR⁶, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R⁴ is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-X″: 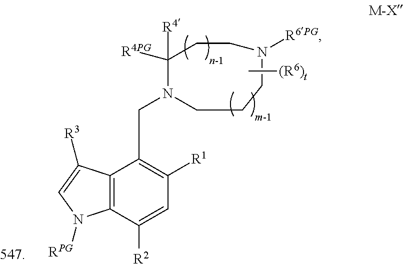
or a pharmaceutically acceptable salt thereof, wherein:
R^{PG} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR⁶, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
R¹ is hydrogen, halogen, hydroxyl, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
R⁷ is hydroxyl, C₁-C₄ alkoxy, amino, or mono- or di-C₁-C₄ alkylamino;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-IV: 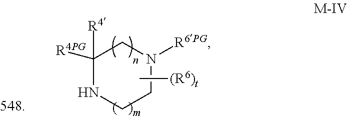
or a pharmaceutically acceptable salt thereof, wherein:
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-IV′: 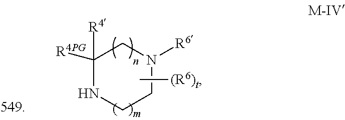
or a pharmaceutically acceptable salt thereof, wherein:
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R⁶ is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-VI: 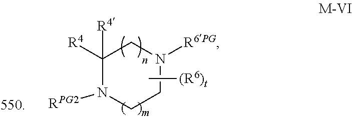
or a pharmaceutically acceptable salt thereof, wherein:
m is 1, or 2;
n is 1, or 2;
R^{PG2} is an amino protecting group;
R⁴ is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R⁵ and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-VII: 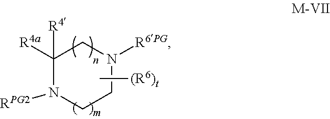
or a salt thereof, wherein:
m is 1, or 2;
n is 1, or 2;
R^{PG2} is an amino protecting group;
R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5a} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-VIII: 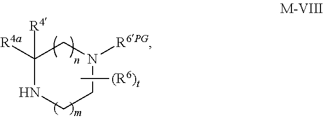
or a salt thereof, wherein:
m is 1, or 2;
n is 1, or 2;
R^{4a} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5a} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-IX: 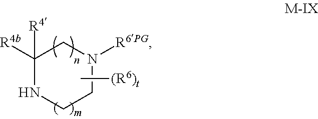
or a salt thereof, wherein:
m is 1, or 2;
n is 1, or 2;
R^{4b} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5b} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5b} is —CH₂OH, —CH₂ CH₂OH, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R⁶ or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-X: 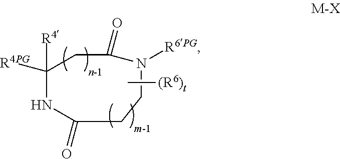
or a salt thereof, wherein:
m is 1, or 2;
n is 1, or 2;
R^{4′} is hydrogen, C₁-C₄ alkyl, or hydroxy C₁-C₄ alkyl;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG}), or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R⁶ or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XI: 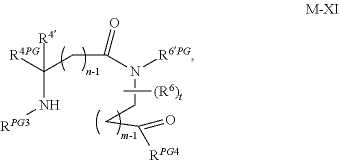
or a salt thereof, wherein:
m is 1, or 2;
n is 1, or 2;
R^{PG3} is —H or an amino protecting group;
R^{PG4} is —OH or —C(O)R^{PG4} is a protected carboxyl group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R⁵ is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R⁵ are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XII: 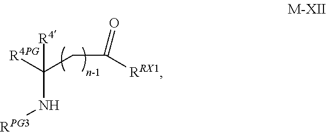
or a salt thereof, wherein:
n is 1, or 2;
R^{PG3} is —H or an amino protecting group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
R^{RX1} is —OH or —C(O)R^{RX1} is an activated carboxyl group;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XIII: 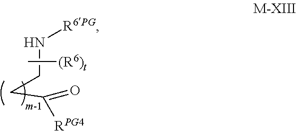
or a salt thereof, wherein:
m is 1, or 2;
R^{PG4} is —OH or —C(O)R^{PG4} is a protected carboxyl group;
R^{6′PG} is R^{6′} or an amino protecting group;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo; or
R^{4′} and one R⁶ are taken together to form C₂-C₄ alkylene;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
t is 0, 1, 2, 3 or 4; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XIV: 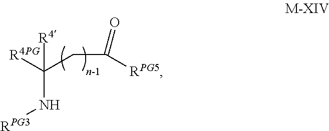
or a salt thereof, wherein:
n is 1, or 2;
R^{PG3} is —H or an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
—C(O)R^{PG5} is protected carboxyl;
R^{4′} is hydrogen, C₁-C₄ alkyl, or hydroxy C₁-C₄ alkyl;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; or
a compound, wherein the compound has the structure of formula M-XV: 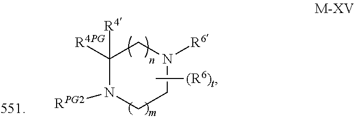
or a pharmaceutically acceptable salt thereof, wherein:
R^{PG2} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-XVI: 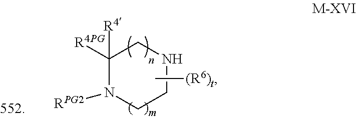
or a pharmaceutically acceptable salt thereof, wherein:
R^{PG2} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG}), or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R⁴ is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound, wherein the compound has the structure of formula M-XVII: 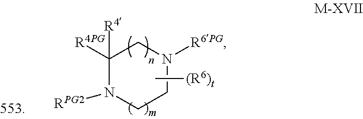
or a pharmaceutically acceptable salt thereof, wherein:
R^{PG2} is an amino protecting group;
R^{4PG} is phenyl, naphthyl, 1,2,3,4-tetrahydronaphthalenyl and 5-10 membered heteroaryl having 1, 2 or 3 ring heteroatoms independently selected from nitrogen, oxygen and sulfur, and wherein the phenyl, naphthyl, 1,2,3,4-tetrahydronaphthalenyl, or heteroaryl is optionally substituted with R^{5PG} and further optionally substituted with one or two R⁵′;
each R^{5′} is independently selected from halogen, C₁-C₄ alkyl, C₁-C₄ alkoxy, hydroxy C₁-C₄ alkyl, hydroxyl, R¹⁰, —OR^{6′}, —N(R⁵¹)₂, halo C₁-C₆ alkyl, cyano, and cyanomethyl; or
two R^{5′} are taken together with their intervening atoms to form a 4-8 membered monocyclic partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁵¹ is independently R⁶′;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxyl, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, oxo
R^{4′} and one R⁶ are taken together to form C₂-C₄ alkylene;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic or bicyclic saturated, partially unsaturated or aromatic ring having 1-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and oxo;
each R⁸ is independently hydroxyl, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and oxo, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

23. A compound selected from Table C-1 or E-1 or a pharmaceutically acceptable salt thereof.

24. A pharmaceutical composition comprising a compound of any one of the preceding claims or a pharmaceutically acceptable salt thereof, and a pharmaceutically acceptable carrier, excipient or diluent.

25. A method for preparing a compound of claim 1, comprising utilizing a compound of claim 22; or
a method, comprising removing one or more protection groups from a compound of formula M-I: 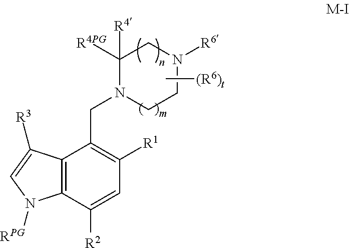
or a salt thereof to provide a compound of formula I or a salt thereof; or
a method comprising contacting an aldehyde or ketone with a compound of formula M-II: 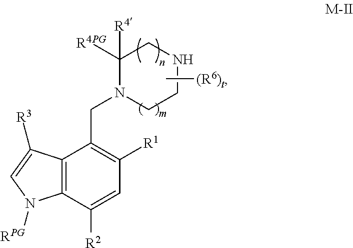
or a salt thereof in the presence of a reducing agent to provide a compound of formula M-I or a salt thereof; or
a method comprising reacting a compound of formula M-II: 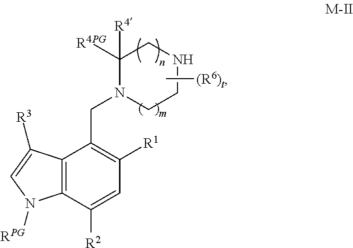
or a salt thereof with a compound having the structure of formula R^{6x3}—R^{6′}, wherein R^{6x3} is a leaving group; or
a method comprising reacting a compound of formula M-II: 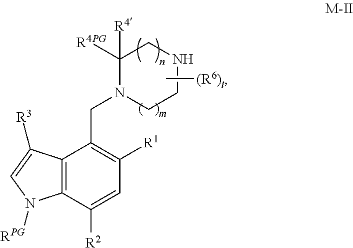
or a salt thereof with an acylating agent; or
a method comprising reacting a compound of formula M-II: 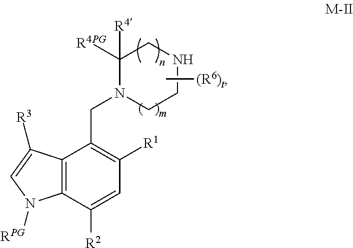
or a salt thereof with a sulfonylation agent; or
a method comprising de-protecting a compound having the structure of M-III: 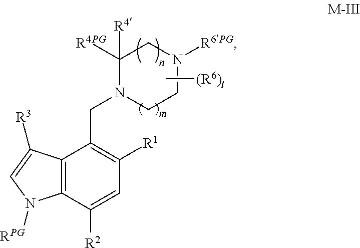
or a salt thereof to provide a compound of formula M-II or a salt thereof; or
a method comprising reacting a compound of formula M-IV: 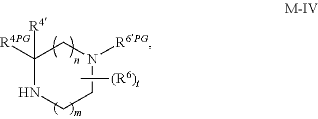
or a salt thereof, with a compound of formula M-V or a salt thereof: 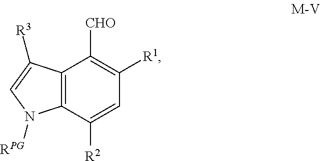
under a condition to provide a compound of formula M-III or a salt thereof; or
a method comprising protecting a group and/or de-protecting a group of a compound of formula M-VI: 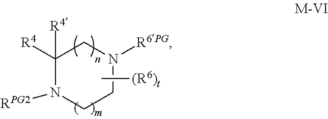
or a salt thereof to provide a compound of formula M-IV or a salt thereof; or
a method comprising converting a compound of formula M-VII: 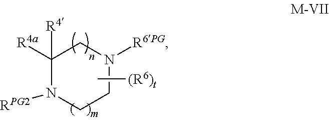
or a salt thereof to provide a compound of formula M-VI or a salt thereof; or
a method comprising protecting a group in a compound of formula M-VIII: 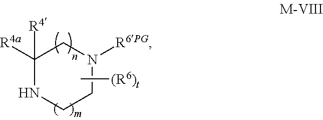
or a salt thereof to provide a compound of formula M-VII or a salt thereof; or
a method comprising converting a compound of formula M-IX: 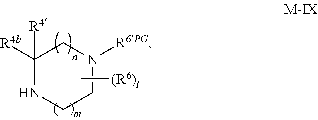
or a salt thereof to provide a compound of formula M-VIII or a salt thereof; or
a method comprising reducing a compound of formula M-X: 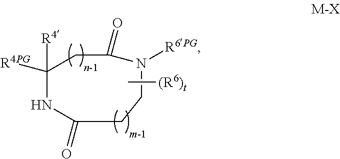
or a salt thereof to provide a compound of formula M-IX or a salt thereof; or
a method comprising converting a compound of formula M-XI: 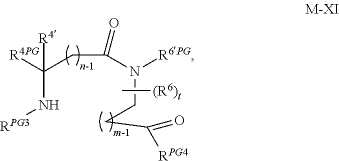
or a salt thereof to provide a compound of formula M-X or a salt thereof; or
a method comprising reacting a compound of formula M-XII: 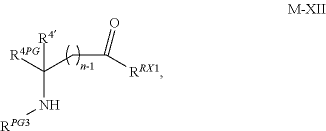
or a salt thereof, with a compound of formula M-XIII: 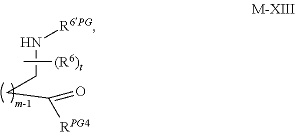
or a salt thereof to provide a compound of formula M-XI or a salt thereof; or
a method comprising converting a compound of formula M-XIV: 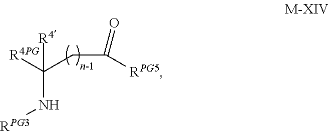
or a salt thereof to a compound of formula M-XII or a salt thereof; or
a method comprising reacting a compound of formula M-IV′: 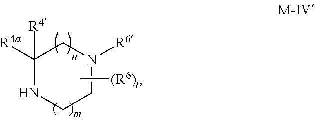
or a salt thereof, with a compound of formula M-V or a salt thereof: 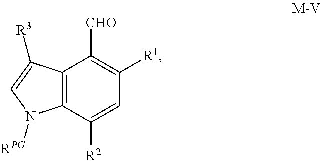
under a condition to provide a compound of formula M-I or a salt thereof; or
a method comprising de-protecting a group of a compound of formula M-XV: 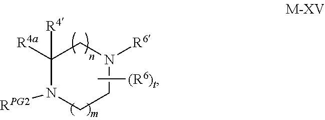
or a salt thereof to provide a compound of formula M-IV′ or a salt thereof; or
a method comprising converting compound of formula M-XVI: 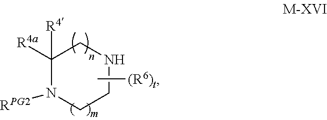
or a salt thereof to a compound of formula M-XV or a salt thereof; or
a method comprising de-protecting a group of a compound of formula M-XVII: 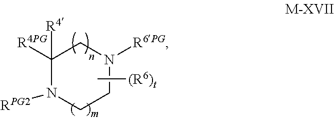
or a salt thereof to a compound of formula M-XVI or a salt thereof; or
a method comprising protecting a group of a compound of formula M-IV or a salt thereof to provide a compound of formula M-XVII or a salt thereof; or
a method, comprising:
reacting a compound having the structure of formula M-XIII or a salt thereof with a compound having the structure of formula M-XII′: 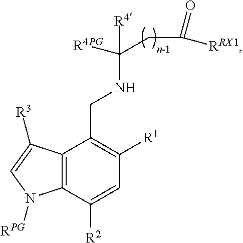
or a salt thereof to provide a compound of formula M-X′: 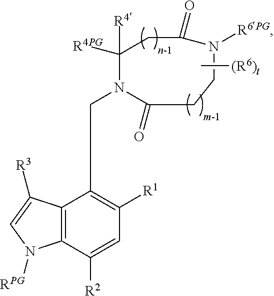
or a salt thereof; or
a method for assessing a compound, comprising utilizing an assay described in the Examples.

26. A method of inhibiting a C3 convertase, comprising contacting a C3 convertase with a compound or composition of any one of the preceding claims; or
a method of modulating complement alternative pathway activity in a subject, wherein the method comprises administering to the subject a therapeutically effective amount of a compound or composition of any one of the preceding claims; or

27. A method of treating a disorder or a disease in a subject mediated by complement activation, wherein the method comprises administering to the subject a therapeutically effective amount of a compound or composition of any one of the preceding claims; or
a method of treating a disorder or a disease in a subject mediated by activation of the complement alternative pathway, wherein the method comprises administering to the subject a therapeutically effective amount of a compound or composition of any one of the preceding claims.

28. The method of claim 27, wherein the subject is suffering from a disease or disorder selected from age-related macular degeneration, geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.

29. A method of treating age related macular degeneration, comprising administering to a subject suffering therefrom a therapeutically effective amount of a compound or composition of any one of the preceding claims.

30. The method of claim 29, wherein the age related macular degeneration is intermediate age-related macular degeneration.

31. The method of any one of claims 29-30, wherein another therapeutic agent is administered such that a subject is exposed to the effects of both the compound and the another therapeutic agent.

32. A compound, composition or method of any one of Embodiments 1-543.
